(19) United States
(12) Patent Application Publication
(10) Pub. No.: US 2024/0343710 A1
(43) Pub. Date: Oct. 17, 2024

(54) HETEROCYCLIC COMPOUNDS AND METHODS OF USE THEREOF

(71) Applicant: 1cBio, Inc., Moraga, CA (US)

(72) Inventors: Jayakanth KANKANALA, Frisco, TX (US); Jeremy D. PETTIGREW, Burnaby (CA); Son Minh PHAM, San Francisco, CA (US)

(21) Appl. No.: 18/583,024

(22) Filed: Feb. 21, 2024

Related U.S. Application Data
(60) Provisional application No. 63/583,602, filed on Sep. 19, 2023.
(60) Provisional application No. 63/486,236, filed on Feb. 21, 2023.

(51) Int. Cl. C07D 401/14; A61K 31/439; A61K 31/444; A61K 31/501; A61K 31/5383; A61P 31/00; C07D 407/14; C07D 487/08; C07D 487/16; C07D 498/16

(52) U.S. Cl. CPC C07D 401/14; A61K 31/439; A61K 31/444; A61K 31/501; A61K 31/5383; A61P 31/00; C07D 407/14; C07D 487/08; C07D 487/16; C07D 498/16

(57) ABSTRACT

The present disclosure relates to compounds of Formula (I):

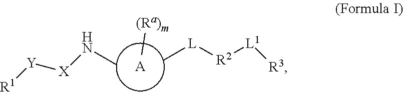

and to their prodrugs, pharmaceutically acceptable salts, pharmaceutical compositions, methods of use, and methods for their preparation. The compounds disclosed herein are useful for modulating PARP1 activity and may be used in the treatment of disorders in which PARP1 activity is implicated, such as cancer.

### CROSS REFERENCE TO RELATED APPLICATIONS

This application claims priority to and the benefit of U.S. Provisional Patent Application Nos. 63/583,602, filed Sep. 19, 2023, and 63/486,236, filed Feb. 21, 2023, the contents of each are incorporated by reference herein in their entirety for all purposes.

### BACKGROUND

The poly(ADP-ribose) polymerase (PARP) family of enzymes comprises 17 members, among which PARP1 plays an important role in the repair of damaged DNA. PARP1 works through binding to single strand breaks (SSBs) and catalyzing the formation of large branched chains of poly(ADP-ribose) (PAR) in an NAD+ dependent manner. This process, known as PARylation, recruits different DNA repair enzymes and facilitates the release of PARP from DNA. All PARP inhibitors approved thus far are NAD+ competitive inhibitors that compete with NAD+ for the PARP catalytic site. They are thought to work by blocking the enzymatic activity of PARP as well as preventing the release of PARP from damaged DNA, an effect known as PARP trapping. Inhibition of PARP leads to the accumulation of DNA SSBs and double strand breaks (DSBs) and an increase in apoptosis.

There are currently four PARP inhibitors marketed in the US, approved for the treatment of BRCA-mutated breast and ovarian cancer. The inhibitors differ from each other in their PARP trapping efficiency. Homologous recombination (HR)-deficient tumor cells with deleterious alterations in BRCA1 or BRCA2 are particularly sensitive to PARP inhibitors.

The approved PARP inhibitors inhibit both PARP1 and PARP2 enzymes. All these first generation PARP1/2 inhibitors have hematologic toxicities like neutropenia, anemia, and thrombocytopenia associated with them. These toxicities are believed to be mediated in large part by PARP2 inhibition.

PARP inhibitors are under testing in combination with immunotherapeutic, chemotherapeutic, and different targeted agents. The various toxicities associated with these agents may be compounded when used in combination with a PARP1/2 inhibitor, so a PARP inhibitor that causes minimal additional toxicity is desirable. Therefore, a highly selective PARP1 inhibitor may have utility not only as a monotherapy for certain cancers, but also as part of combination therapies, and would fill an unmet need for effective, more tolerable treatments for patients suffering from cancer.

### SUMMARY

In some aspects, the present disclosure relates to small molecule, potent inhibitors of poly [ADP-ribose] polymerase 1 (PARP1), designed for the treatment of cancer. It is hypothesized that PARP1-selective inhibitors will have an improved therapeutic index and reduced toxicity as compared to first generation PARP inhibitors.

In some aspects, the present disclosure provides a compound of Formula (I):

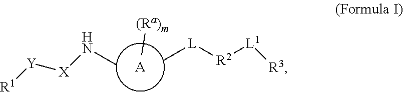

or a pharmaceutically acceptable salt, thereof, wherein
\textcircled{A} is C₃₋₁₀ cycloalkylene, C₆₋₁₀ arylene, 3- to 10-membered heterocycloalkylene, or 5- to 10-membered heteroarylene;
m is 0, 1, 2, 3, 4, or 5;
each R^{a} independently is halogen, —CN, —OH, —NH₂, —O—(C₁₋₆alkyl), —NH(C₁₋₆alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆ alkyl), —NH(C₁₋₆ alkyl), —N(C₁₋₆ alkyl)₂, or —C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{a}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
X is —C(═O)—, —S(═O)₂—;
Y is a bond, —O—, —S—, —NH—, or C₁₋₆ alkylene, wherein the C₁₋₆ alkylene is optionally substituted with one or more oxo, halogen, —CN, —OH, —NH₂, C₁₋₆ alkyl, or two C₁₋₆ alkyl that combine with the atom or atoms to which they are attached, to form a C₃₋₁₀ cycloalkyl group;
R¹ is H, halogen, —CN, —OH, —NH₂, —C₁₋₆ alkyl, —O—(C₁₋₆ alkyl), —NH(C₁₋₆ alkyl), —N(C₁₋₆ alkyl)(C₂₋₆ alkyl), C₃₋₁₀ cycloalkyl, C₆₋₁₀ aryl, 3- to 10-membered heterocycloalkyl, or 5- to 10-membered heteroaryl, wherein the —C₁₋₆ alkyl, —O—(C₁₋₆ alkyl), —NH(C₁₋₆ alkyl), or —N(C₁₋₆ alkyl)₂, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₁₋₁₀ cycloalkyl, or 3- to 10-membered heterocycloalkyl, or 5- to 10-membered heteroaryl, and the C₃₋₁₀cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
L is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R² is C₃₋₁₀ cycloalkylene, C₆₋₁₀ arylene, 3- to 10-membered heterocycloalkylene, or 5- to 10-membered heteroarylene, wherein the C₃₋₁₀ cycloalkylene, C₆₋₁₀ arylene, 3- to 10-membered heterocycloalkylene, or 5- to 10-membered heteroarylene is optionally substituted with one or more R^{b};
each R^{a} independently is halogen, —CN, —OH, —NH₂, —O—(C₁₋₆alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆alkyl), —NH(C₁₋₆alkyl), —N(C₁₋₆alkyl)₂, C₁₋₆alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ to aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{b}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
L¹ is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₁₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R³ is C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R;
each R^{c} independently is halogen, —CN, —OH, —NH₂, —C(═O)—H, —C(═O)—NH₂, —NH—C(═O)—H, —NH—(C═O)—NH₂, —C(═O)—CN, —NH—C(═O)—CN, —C(═O)—NH—CN, —O—(C₁₋₆ alkyl), —NH(C₁₋₆ alkyl), —N(C₁₋₆alkyl)₂, —C(═O)—(C₁₋₆alkyl), —C(═O)—NH—(C₁₋₆alkyl), —NH—C(═O)—(C₁₋₆alkyl), —NH—(C═O)—NH—(C₁₋₆ alkyl), —NH—(C═O)—O—(C₁₋₆ alkyl), —C(═O)—NH—(C₃₋₁₀cycloalkyl), —C(═O)—NH-(3- to 10-membered heterocycloalkyl), C₁₋₆alkyl, C₂₋₆alkenyl, C₂₋₆alkynyl, C₃₋₁₀cycloalkyl, C₆₋₁₀ aryl, 3- to 10-membered heterocycloalkyl, or 5- to 10-membered heteroaryl, or two R^{c} combine to form oxo, wherein the —O—(C₁₋₆alkyl), —NH(C₁₋₆alkyl), —N(C₁₋₆alkyl)₂, —C(═O)—(C₁₋₆alkyl), —C(═O)—NH—(C₁₋₆ alkyl), —NH—C(═O)—(C₁₋₆alkyl), —NH—(C═O)—NH—(C₁₋₆alkyl), or —NH—(C═O)—O—(C₁₋₆ alkyl), is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
or one R^{b} and one R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R^{bc}; and
each R^{bc} independently is oxo, deuterium, halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

In some aspects, the present disclosure provides a compound obtainable by, or obtained by, a method for preparing a compound as described herein (e.g., a method comprising one or more steps described in Schemes 1-12).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-55).

In some aspects, the present disclosure provides a method of modulating PARP1 activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating PARP1 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating PARP1 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

In some embodiments, the present disclosure provides a compound having any one of the structures shown in Table 1, or a pharmaceutically acceptable salt thereof.

In some embodiments, the present disclosure provides a pharmaceutical composition comprising a compound of Formulae I-XLIV and a pharmaceutically acceptable excipient.

In some embodiments, the present disclosure is directed to a method of treating a cancer with a compound of Formulae I-XLIV or Table 1 or a pharmaceutical composition comprising a compound of Formulae I-XLIV or Table 1 to a subject in need thereof.

In some embodiments, the present disclosure is directed to a method of treating a cancer with a BRCAness phenotype comprising administering a compound of Formulae I-XLIV or Table 1 or a pharmaceutical composition thereof to subject in need thereof.

In some embodiments, the present disclosure is directed to a method of treating a breast cancer, ovarian cancer, prostate cancer, or pancreatic cancer comprising administering a compound of Formulae I-XLIV or Table 1 to a subject in need thereof.

In some embodiments, the present disclosure provides a method of treating a HR-deficient cancer comprising administering a compound of Formulae I-XLIV or Table 1 to a subject in need thereof.

In some embodiments, the present disclosure provides a method of treating a BRCA1- or BRCA2-mutant cancer, comprising administering an effective amount of the compound of Formulae I-XLIV or Table 1, or the pharmaceutical composition thereof, to a subject in need thereof.

In some embodiments, the present disclosure provides a method of treating a HR-deficient cancer, a BRCA1- or BRCA2-mutant cancer, or a cancer with a BRCAness phenotype in a subject, the method comprising administering an effective amount of the compound of Formulae I-XLIV or Table 1 or a pharmaceutical composition thereof, to a subject in need thereof.

In some embodiments, the cancer with a BRCAness phenotype is selected from breast cancer, ovarian cancer, prostate cancer, or pancreatic cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to heterocyclic derivatives, prodrugs, and pharmaceutically acceptable salts thereof, which may modulate PARP1 activity and are accordingly useful in methods of treatment of the human or animal body. The present disclosure also relates to processes for the preparation of these compounds, to pharmaceutical compositions comprising them and to their use in the treatment of disorders in which PARP1 is implicated.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables L, R², and L¹ are described herein, the disclosure may be interpreted as excluding structures for non-operable compound caused by certain combinations of variables L, R², and L¹ (e.g., when each of L, R², and L¹ is —O—).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkylene” refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂), butylene (—CH₂CH₂CH₂CH₂—), pentylene (—CH₂CH₂CH₂CH₂CH₂—), hexylene (CH₂CH₂CH₂CH₂CH₂CH₂—), and the like.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkenylene” refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—).

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “alkynylene” refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, haloalkoxy, heterocycloalkyl, aryl, heterocyclyl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “cycloalkylene” refers to a cycloalkyl group wherein two hydrogens are removed to provide a divalent radical. It is understood that the two hydrogens, prior to removal, can be attached to same or different atoms in the cycloalkyl group.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “heterocycloalkylene” refers to a heterocycloalkyl group wherein two hydrogens are removed to provide a divalent radical. It is understood that the two hydrogens, prior to removal, can be attached to same or different atoms in the cycloalkyl group.

It is understood that when a variable has two attachments to the rest of the formula of the compound, the two attachments could be at the same atom or different atoms of the variable. For example, when a variable (e.g., variable X) is cycloalkyl or heterocycloalkyl, and has two attachments to the rest of the formula of the compound, the two attachments could be at the same atom or different atoms of the cycloalkyl or heterocycloalkyl.

As used herein, the term “aryl” includes groups with aromaticity, including “conjugated”, or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. For example, an aryl is phenyl.

As used herein, the term “arylene” refers to an aryl group wherein two hydrogens are removed to provide a divalent radical.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

As used herein, the term “heteroarylene” refers to a heteroaryl group wherein two hydrogens are removed to provide a divalent radical.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3′ edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze drying that yields a powder of the active ingredient plus any additional desired ingredient from a previously sterile filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight (w/w). Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As used herein, the phrase “compound of the disclosure” refers to those compounds which are disclosed herein, both generically and specifically.

### Compounds of the Present Disclosure

In some aspects, the present disclosure provides a compound of Formula (I):

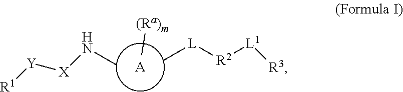

or a pharmaceutically acceptable salt, thereof, wherein
\textcircled{A} is C₃₋₁₀ cycloalkylene, C₆₋₁₀ arylene, 3- to 10-membered heterocycloalkylene, or 5- to 10-membered heteroarylene;
m is 0, 1, 2, 3, 4, or 5;
each R^{a} independently is halogen, —CN, —OH, —NH₂, —O—(C₁₋₆alkyl), —NH(C₁₋₆alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆ alkyl), —NH(C₁₋₆ alkyl), —N(C₁₋₆ alkyl)₂, or —C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₁₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
X is —C(═O)—, —S(═O)₂—;
Y is a bond, —O—, —S—, —NH—, or C₁₋₆ alkylene, wherein the C₁₋₆ alkylene is optionally substituted with one or more oxo, halogen, —CN, —OH, —NH₂, C₁₋₆ alkyl, or two C₁₋₆ alkyl that combine, with the atom or atoms to which they are attached, to form a C₃₋₁₀ cycloalkyl group;
R¹ is H, halogen, —CN, —OH, —NH₂, —C₁₋₆ alkyl, —O—(C₁₋₆ alkyl), —NH(C₁₋₆ alkyl), —N(C₁₋₆ alkyl)(C₂₋₆ alkyl), C₃₋₁₀ cycloalkyl, C₆₋₁₀ aryl, 3- to 10-membered heterocycloalkyl, or 5- to 10-membered heteroaryl, wherein the —C₁₋₆ alkyl, —O—(C₁₋₆ alkyl), —NH(C₁₋₆ alkyl), or —N(C₁₋₆ alkyl)₂, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, or 5- to 10-membered heteroaryl, and the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
L is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R² is C₃₋₁₀ cycloalkylene, C₆₋₁₀ arylene, 3- to 10-membered heterocycloalkylene, or 5- to 10-membered heteroarylene, wherein the C₃₋₁₀ cycloalkylene, C₆₋₁₀ arylene, 3- to 10-membered heterocycloalkylene, or 5- to 10-membered heteroarylene is optionally substituted with one or more R^{b};
each R^{b} independently is halogen, —CN, —OH, —NH₂, —O—(C₁₋₆alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{b} combine to form oxo, wherein the —O—(C₁₋₆alkyl), —NH(C₁₋₆alkyl), —N(C₁₋₆alkyl)₂, C₁₋₆alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{b}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
L¹ is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R³ is C₃₋₁₀ cycloalkyl, C₆₋₁₀ aryl, 3- to 10-membered heterocycloalkyl, or 5- to 10-membered heteroaryl, wherein the C₃₋₁₀cycloalkyl, C₆₋₁₀ aryl, 3- to 10-membered heterocycloalkyl, or 5- to 10-membered heteroaryl is optionally substituted with one or more R;
each R^{c} independently is halogen, —CN, —OH, —NH₂, —C(═O)—H, —C(═O)—NH₂, —NH—C(═O)—H, —NH—(C═O)—NH₂, —C(═O)—CN, —NH—C(═O)—CN, —C(═O)—NH—CN, —O—(C₁₋₆ alkyl), —NH(C₁₋₆ alkyl), —N(C₁₋₆ alkyl)₂, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), —NH—(C═O)—O—(C₁₋₆ alkyl), —C(═O)—NH—(C₃₋₁₀ cycloalkyl), —C(═O)—NH-(3- to 10-membered heterocycloalkyl), C₁₋₆alkyl, C₂₋₆alkenyl, C₂₋₆alkynyl, C₃₋₁₀ cycloalkyl, C₆₋₁₀ aryl, 3- to 10-membered heterocycloalkyl, or 5- to 10-membered heteroaryl, or two R^{c} combine to form oxo, wherein the —O—(C₁₋₆ alkyl), —NH(C₁₋₆ alkyl), —N(C₁₋₆alkyl)₂, —C(═O)—(C₁₋₆alkyl), —C(═O)—NH—(C₁₋₆alkyl), —NH—C(═O)—(C₁₋₆ alkyl), —NH—(C═O)—NH—(C₁₋₆alkyl), or —NH—(C═O)—O—(C₁₋₆ alkyl), is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆- to aryl, or 5- to 10-membered heteroaryl;
or two R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
or one R^{b} and one R, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R^{bc}; and
each R^{bc} independently is oxo, deuterium, halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, halogen, —CN, —OH, or —NH₂.

In some aspects, the present disclosure provides a compound of Formula (I):

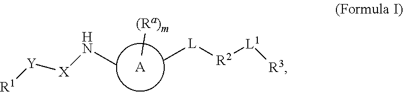

or a pharmaceutically acceptable salt, thereof, wherein
\textcircled{A} is C₃₋₁₀ cycloalkylene, C₆₋₁₀ arylene, 3- to 10-membered heterocycloalkylene, or 5- to 10-membered heteroarylene;
m is 0, 1, 2, 3, 4, or 5;
each R^{a} independently is halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆ alkyl), —NH(C₁₋₆ alkyl), —N(C₁₋₆ alkyl)₂, or —C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₁₋₁₀ cycloalkyl, or 3- to 10-membered heterocycloalkyl, C₆₋₁₀ aryl, or 5- to 10-membered heteroaryl;
or two R^{a}, together with the intervening atoms they are attached to, form C₃₋₁₀ cycloalkyl, C₆- to aryl, 3- to 10-membered heterocycloalkyl, or 5- to 10-membered heteroaryl, wherein the C₁₋₁₀ cycloalkyl, C₆- to aryl, 3- to 10-membered heterocycloalkyl, or 5- to 10-membered heteroaryl is optionally substituted with one or more oxo, deuterium, halogen, —CN, —OH, or —NH₂;
X is —C(═O)—, —S(═O)₂—;
Y is a bond, —O—, —S—, —NH—, or C₁₋₆ alkylene, wherein the C₁₋₆ alkylene is optionally substituted with one or more oxo, halogen, —CN, —OH, —NH₂, C₁₋₆ alkyl, or two C₁₋₆ alkyl that combine, with the atom or atoms to which they are attached, to form a C₃₋₁₀ cycloalkyl group;
R¹ is H halogen, —CN, —OH, —NH₂, —C₁₋₆ alkyl, —O—(C₁₋₆ alkyl), —NH(C₁₋₆ alkyl), —N(C₁₋₆ alkyl)(C₂₋₆ alkyl), C₃₋₁₀ cycloalkyl, C₆₋₁₀ aryl, 3- to 10-membered heterocycloalkyl, or 5- to 10-membered heteroaryl, wherein the —C₁₋₆ alkyl, —O—(C₁₋₆ alkyl), —NH(C₁₋₆ alkyl), or —N(C₁₋₆ alkyl)₂, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, or 5- to 10-membered heteroaryl, and the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
L is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R² is C₃₋₁₀ cycloalkylene, C₆₋₁₀ arylene, 3- to 10-membered heterocycloalkylene, or 5- to 10-membered heteroarylene, wherein the C₃₋₁₀ cycloalkylene, C₆₋₁₀ arylene, 3- to 10-membered heterocycloalkylene, or 5- to 10-membered heteroarylene is optionally substituted with one or more R^{b};
each R^{b} independently is halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{b} combine to form oxo, wherein the —O—(C₁₋₆alkyl), —NH(C₁₋₆alkyl), —N(C₁₋₆alkyl)₂, C₁₋₆alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₁₋₁₀ cycloalkyl, or 3- to 10-membered heterocycloalkyl, C₆- to aryl, or 5- to 10-membered heteroaryl;
or two R^{b}, together with the intervening atoms they are attached to, form C₃₋₁₀ cycloalkyl, C₆- to aryl, 3- to 10-membered heterocycloalkyl, or 5- to 10-membered heteroaryl, wherein the C₃₋₁₀ cycloalkyl, C₆- to aryl, 3- to 10-membered heterocycloalkyl, or 5- to 10-membered heteroaryl is optionally substituted with one or more oxo, deuterium, halogen, —CN, —OH, or —NH₂;
L¹ is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R³ is C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R;
each R^{bc} independently is halogen, —CN, —OH, —NH₂, —C(═O)—H, —C(═O)—NH₂, —NH—C(═O)—H, —NH—(C═O)—NH₂, —C(═O)—CN, —NH—C(═O)—CN, —C(═O)—NH—CN, —O—(C₁₋₆ alkyl), —NH(C₁₋₆ alkyl), —N(C₁₋₆alkyl)₂, —C(═O)—(C₁₋₆alkyl), —C(═O)—NH—(C₁₋₆alkyl), —NH—C(═O)—(C₁₋₆alkyl), —NH—(C═O)—NH—(C₁₋₆ alkyl), —NH—(C═O)—O—(C₁₋₆ alkyl), —C(═O)—NH—(C₃₋₁₀cycloalkyl), —C(═O)—NH-(3- to 10-membered heterocycloalkyl), C₁₋₆alkyl, C₂₋₆alkenyl, C₂₋₆alkynyl, C₃₋₁₀ cycloalkyl, C₆₋₁₀ aryl, 3- to 10-membered heterocycloalkyl, or 5- to 10-membered heteroaryl, or two R^{c} combine to form oxo, wherein the —O—(C₁₋₆alkyl), —NH(C₁₋₆ alkyl), —N(C₁₋₆ alkyl)₂, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆alkyl), or —NH—(C═O)—O—(C₁₋₆ alkyl), is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
or one R^{b} and one R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R^{bc}; and
each R^{bc} independently is oxo, deuterium, halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

### Exemplary Formulae of the Compounds

In some embodiments, the compound is of Formula (II),

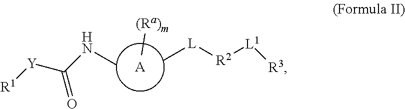

or a pharmaceutically acceptable salt thereof, wherein \textcircled{A}, R¹, R², R³, Y, R^{a}, m, L and L¹ are as detailed herein.

In some embodiments, the compound is of Formula (III),

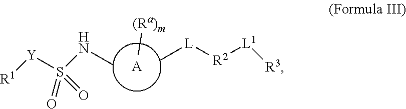

or a pharmaceutically acceptable salt thereof, wherein \textcircled{A}, R¹, R², R³, Y, R^{a}, m, L and L¹ are as detailed herein.

In some embodiments the compound is of Formula (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), (II-u), (II-v), (II-w), (II-x), (II-y), (II-z), (II-aa), (II-ab), (TI-ac), (TI-ad), (II-ae), (II-af), (II-ag), (II-ah), (II-ai), (II-aj), (II-ak), or (II-al), or a pharmaceutically acceptable salt thereof, wherein R¹, R², R³, Y, R^{a}, m, L and L¹ are as detailed herein.

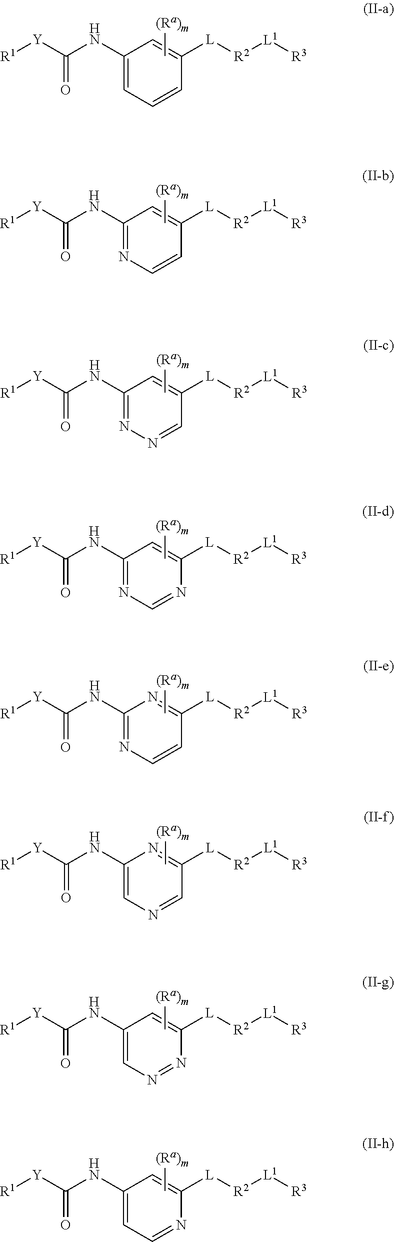

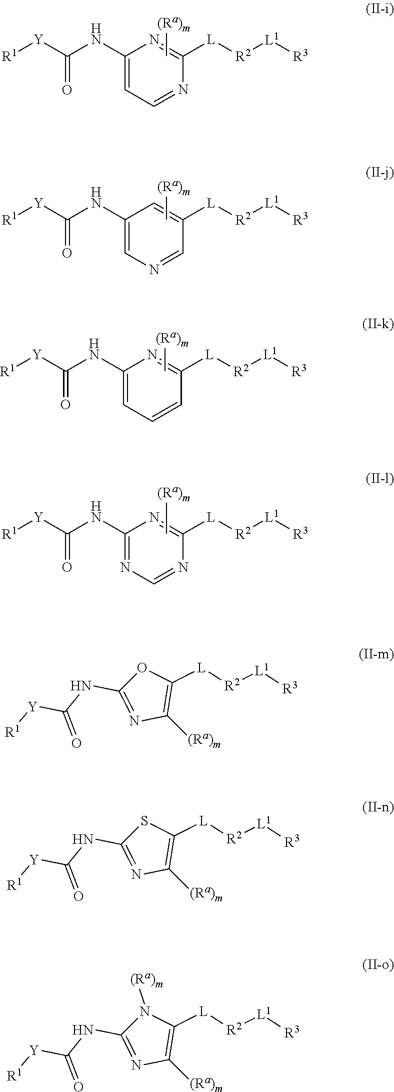

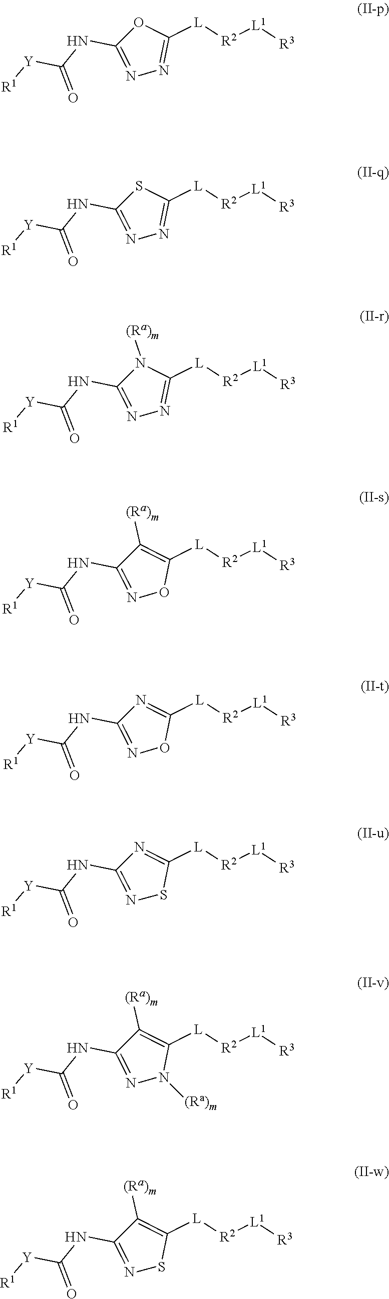

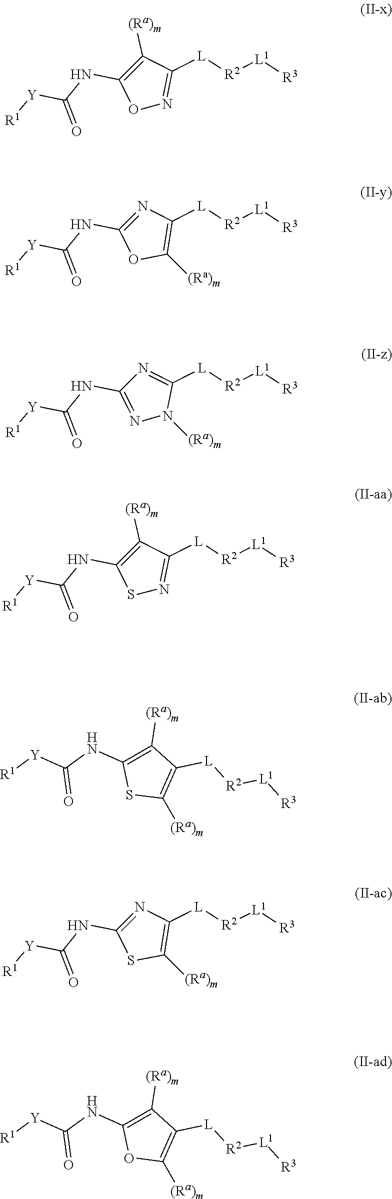

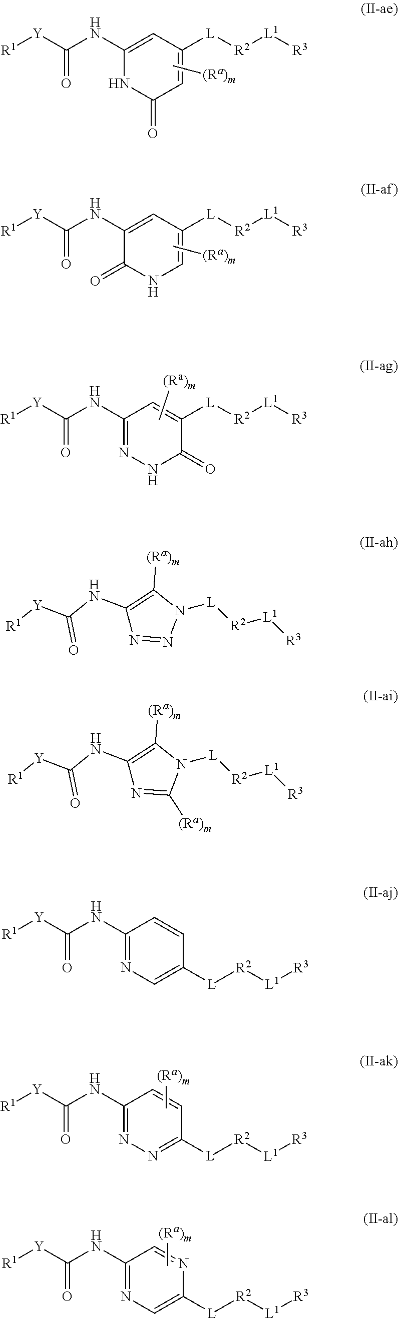

In some embodiments the compound is of Formula (IIa-a), (III-b), (III-c), (III-d), (III-e), ae), (III-af), (III-ag), (III-ah), (III-ai), (III-aj), (III-ak), or (III-al), or a pharmaceutically acceptable salt thereof, wherein R¹, R², R³, Y, R^{a}, m, L and L¹ are as detailed herein.

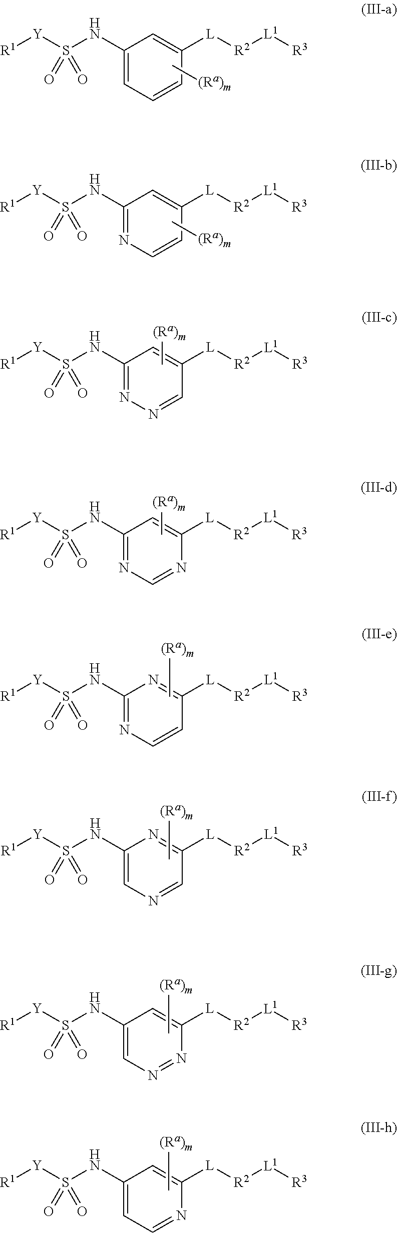

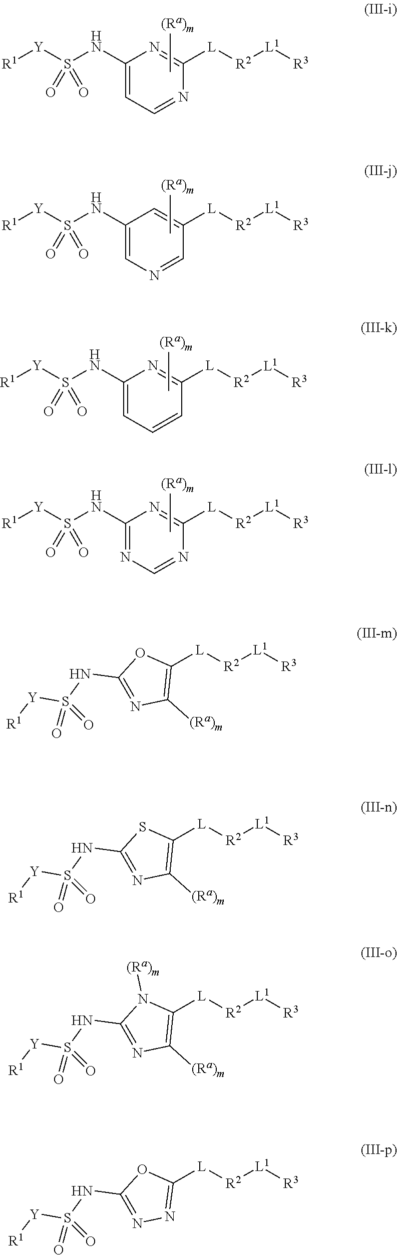

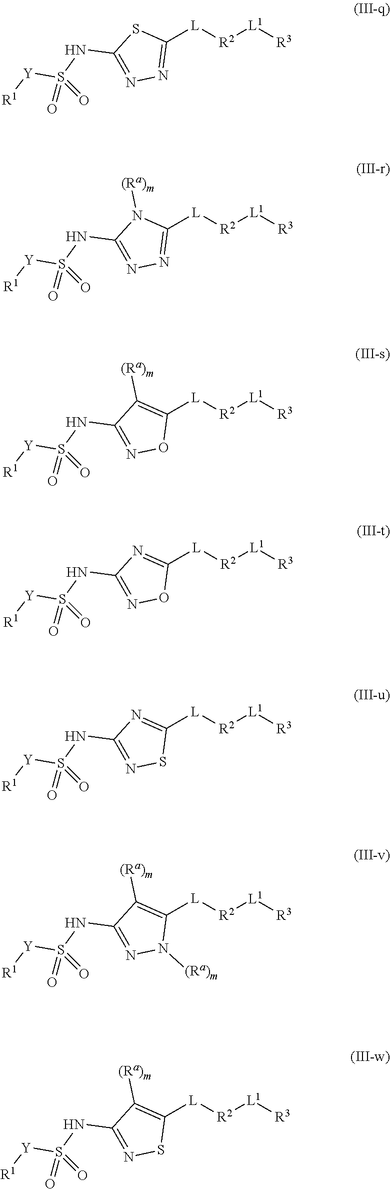

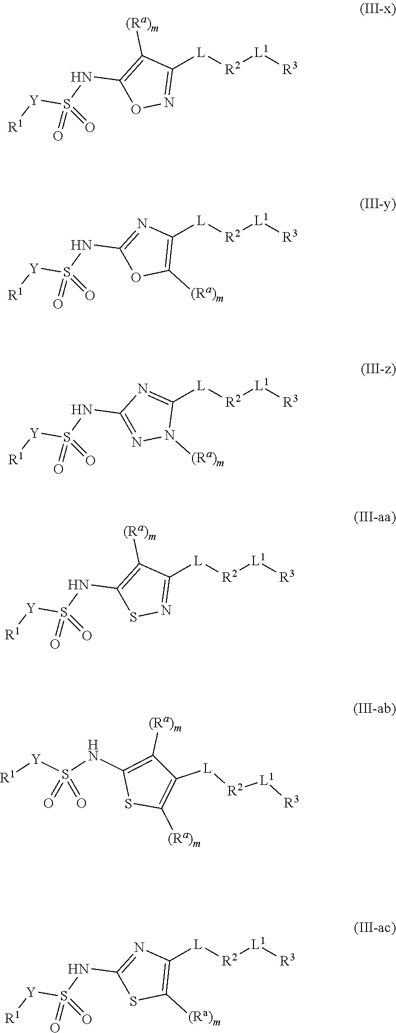

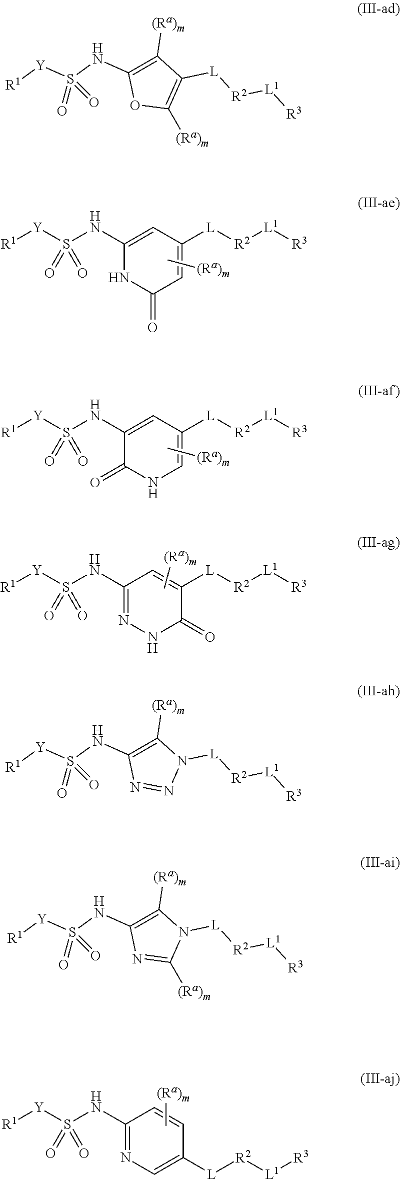

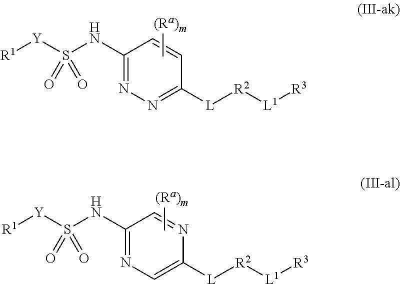

In some embodiments, the compound is of Formula (IV′),

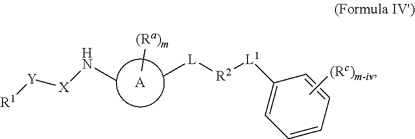

or a pharmaceutically acceptable salt thereof, wherein \textcircled{A}, R¹, R², X, Y, R^{a}, R^{c}, m, L and L¹ are as detailed herein; and
m-iv is 0 to 5.

In some embodiments, the compound of Formula (IV′) is of Formula (IV),

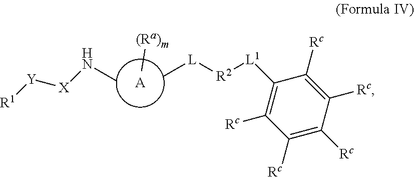

or a pharmaceutically acceptable salt thereof, wherein \textcircled{A}, R¹, R², X, Y, R^{a}, R^{c}, m, L and L¹ are as detailed herein.

In some embodiments, the compound is of Formula (V′),

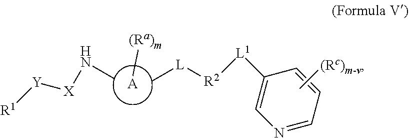

Or a pharmaceutically acceptable salt thereof, wherein \textcircled{A}, R¹, R², X, Y, R^{a}, R^{c}, m, L and L¹ are as detailed herein; and
m-v is 0 to 4.

In some embodiments, the compound of Formula (V′) is of Formula (V),

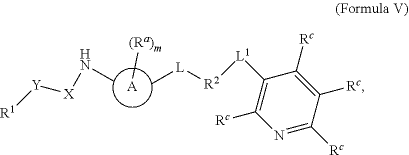

or a pharmaceutically acceptable salt thereof, wherein \textcircled{A}, R¹, R², X, Y, R^{a}, R^{c}, m, L and L¹ are as detailed herein.

In some embodiments, the compound is of Formula (VI′),

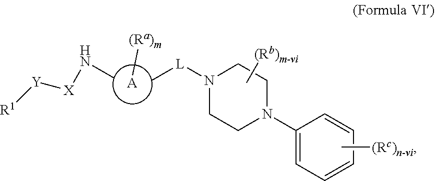

or a pharmaceutically acceptable salt thereof, wherein \textcircled{A}, R¹, X, Y, R^{a}, R^{b}, R, m and L are as detailed herein;
m-vi is 0 to 8; and
n-vi is 0 to 5.

In some embodiments, the compound of Formula (VI′) is of Formula (VI),

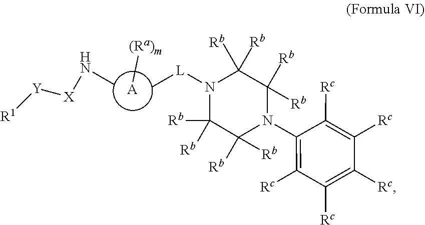

or a pharmaceutically acceptable salt thereof, wherein \textcircled{A}, R¹, X, Y, R^{a}, R^{b}, R^{c}, m and L are as detailed herein.

In some embodiments, the compound is of Formula (VII′),

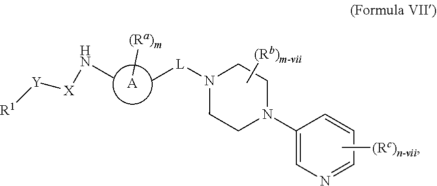

or a pharmaceutically acceptable salt thereof, wherein \textcircled{A}, R¹, X, Y, R^{a}, R^{b}, R^{c}, m and L are as detailed herein;
m-vii is 0 to 8; and
n-vii is 0 to 5.

In some embodiments, the compound of Formula (VII′) is of Formula (VII),

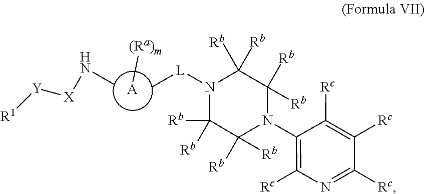

or a pharmaceutically acceptable salt thereof, wherein \textcircled{A}, R¹, X, Y, R^{a}, R^{b}, R^{c}, m and L are as detailed herein.

In some embodiments, the compound is of Formula (VIII′),

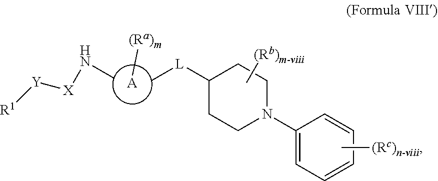

or a pharmaceutically acceptable salt thereof, wherein \textcircled{A}, R¹, X, Y, R^{a}, R^{b}, R^{c}, m and L are as detailed herein;
m-viii is 0 to 9; and
n-viii is 0 to 5.

In some embodiments, the compound of Formula (VIII′) is of Formula (VIII),

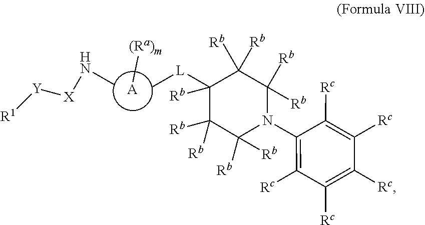

or a pharmaceutically acceptable salt thereof, wherein \textcircled{A}, R¹, X, Y, R^{a}, R^{b}, R^{c}, m and L are as detailed herein.

In some embodiments, the compound is of Formula (IX′),

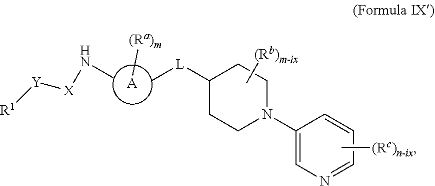

or a pharmaceutically acceptable salt thereof, wherein \textcircled{A}, R¹, X, Y, R^{a}, R^{b}, R^{c}, m and L are as detailed herein;
m-ix is 0 to 9; and
n-ix is 0 to 4.

In some embodiments, the compound of Formula (IX′) is of Formula (IX),

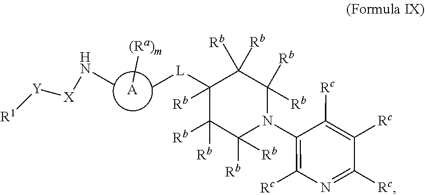

or a pharmaceutically acceptable salt thereof, wherein \textcircled{A}, R¹, X, Y, R^{a}, R^{b}, R^{c}, m and L are as detailed herein.

In some embodiments, the compound is of Formula (X′),

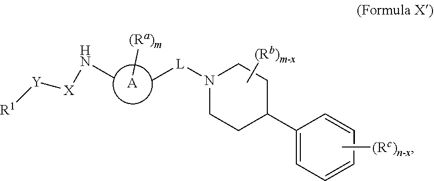

or a pharmaceutically acceptable salt thereof, wherein \textcircled{A}, R¹, X, Y, R^{a}, R^{b}, R, m and L are as detailed herein;
m-x is 0 to 9; and
n-x is 0 to 5.

In some embodiments, the compound of Formula (X′) is of Formula (X),

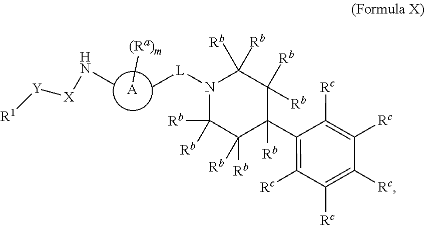

or a pharmaceutically acceptable salt thereof, wherein \textcircled{A}, R¹, X, Y, R^{a}, R^{b}, R^{c}, m and L are as detailed herein.

In some embodiments, the compound is of Formula (XI′),

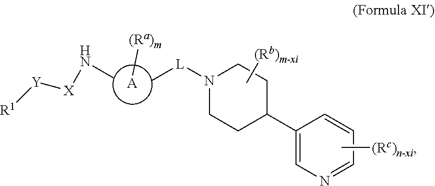

or a pharmaceutically acceptable salt thereof, wherein \textcircled{A}, R¹, X, Y, R^{a}, R^{b}, R^{c}, m and L are as detailed herein;
m-xi is 0 to 9; and
n-xi is 0 to 4.

In some embodiments, the compound of Formula (XI′) is of Formula (XI),

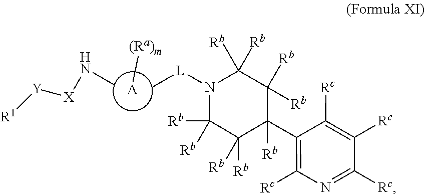

or a pharmaceutically acceptable salt thereof, wherein \textcircled{A}, R¹, X, Y, R^{a}, R^{b}, R^{c}, m and L are as detailed herein.

In some embodiments, the compound is of Formula (XII),

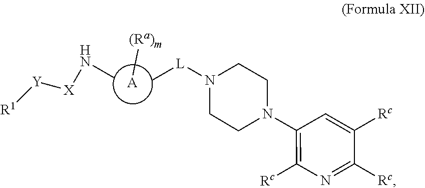

or a pharmaceutically acceptable salt thereof, wherein \textcircled{A}, R¹, X, Y, R^{a}, R^{c}, m and L are as detailed herein.

In some embodiments, the compound is of Formula (XIII),

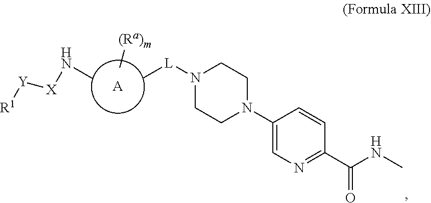

or a pharmaceutically acceptable salt thereof, wherein \textcircled{A}, R¹, X, Y, R^{a}, m and L are as detailed herein.

In some embodiments, the compound is of Formula (XIV),

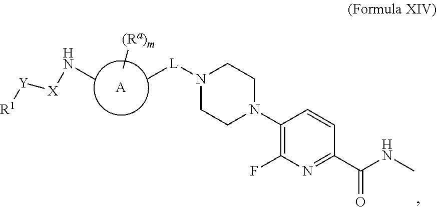

or a pharmaceutically acceptable salt thereof, wherein \textcircled{A}, R¹, X, Y, R^{a}, m and L are as detailed herein.

In some embodiments, the compound is of Formula (XV),

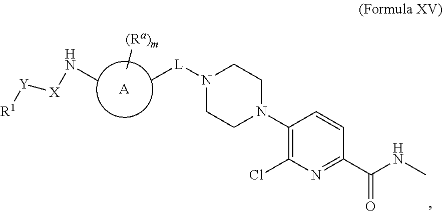

or a pharmaceutically acceptable salt thereof, wherein \textcircled{A}, R¹, X, Y, R^{a}, m and L are as detailed herein.

In some embodiments, the compound is of Formula (XVI),

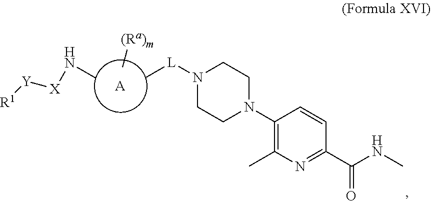

or a pharmaceutically acceptable salt thereof, wherein \textcircled{A}, R¹, X, Y, R^{a}, m and L are as detailed herein.

In some embodiments, the compound is of Formula (XVII),

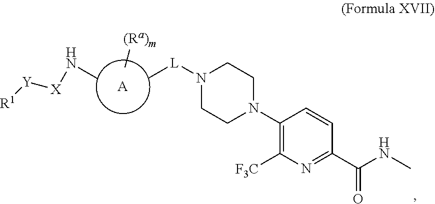

or a pharmaceutically acceptable salt thereof, wherein \textcircled{A}, R¹, X, Y, R^{a}, m and L are as detailed herein.

In some embodiments, the compound is of Formula (XVIII),

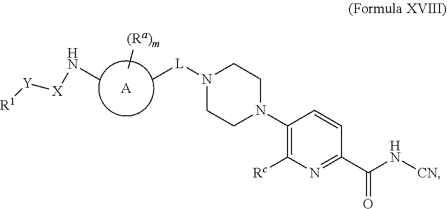

or a pharmaceutically acceptable salt thereof, wherein \textcircled{A}, R¹, X, Y, R^{a}, R^{c}, m and L are as detailed herein.

In some embodiments, the compound is of Formula (XIX),

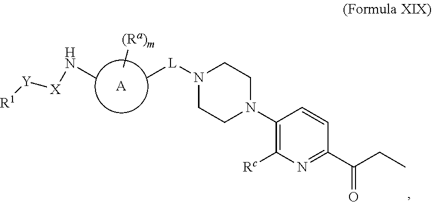

or a pharmaceutically acceptable salt thereof, wherein \textcircled{A}, R¹, X, Y, R^{a}, R^{c}, m and L are as detailed herein.

In some embodiments, the compound is of Formula (XX),

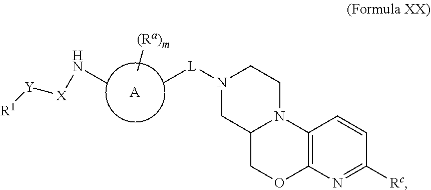

or a pharmaceutically acceptable salt thereof, wherein \textcircled{A}, R¹, X, Y, R^{a}, R^{c}, m and L are as detailed herein.

In some embodiments, the compound is of Formula (XXI),

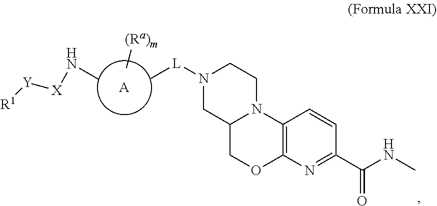

or a pharmaceutically acceptable salt thereof, wherein \textcircled{A}, R¹, X, Y, R^{a}, m and L are as detailed herein.

In some embodiments, the compound is of Formula (XXII),

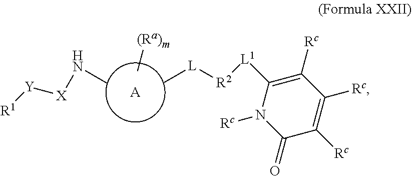

or a pharmaceutically acceptable salt thereof, wherein \textcircled{A}, R¹, R², X, Y, R^{a}, R^{c}, m, L and L are as detailed herein.

In some embodiments the compound is of Formula (XXIII′),

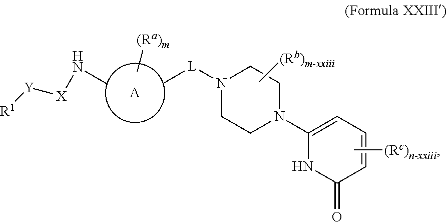

or a pharmaceutically acceptable salt thereof, wherein \textcircled{A}, R¹, X, Y, R^{a}, R^{b}, R^{c}, m and L are as detailed herein;
m-xxiii is 0 to 8; and
n-xxiii is 0 to 4.

In some embodiments, the compound of Formula (XXIII′) is of Formula (XXIII),

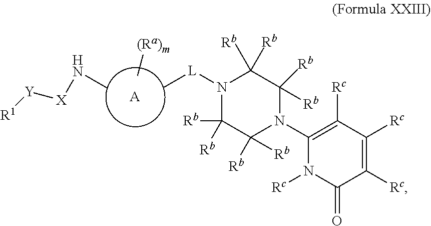

or a pharmaceutically acceptable salt thereof, wherein \textcircled{A}, R¹, X, Y, R^{a}, R^{b}, R^{c}, m and L are as detailed herein.

In some embodiments, the compound is of Formula (XXIV),

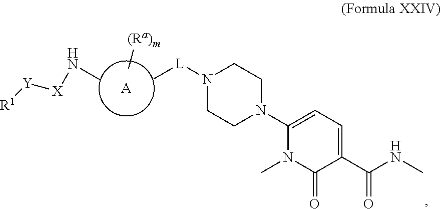

or a pharmaceutically acceptable salt thereof, wherein \textcircled{A}, R¹, X, Y, R^{a}, m and L are as detailed herein.

In some embodiments, the compound is of Formula (XXV),

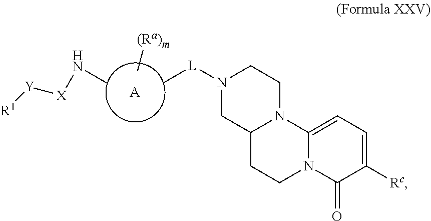

or a pharmaceutically acceptable salt thereof, wherein \textcircled{A}, R¹, X, Y, R^{a}, R^{c}, m and L are as detailed herein.

In some embodiments, the compound is of Formula (XXVI),

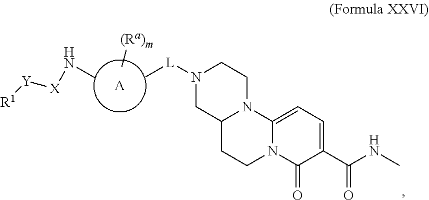

or a pharmaceutically acceptable salt thereof, wherein \textcircled{A}, R¹, X, Y, R^{a}, m and L are as detailed herein.

In some embodiments, the compound is of Formula (XXVII),

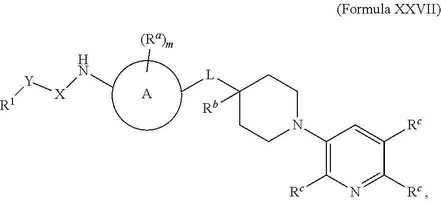

or a pharmaceutically acceptable salt thereof, wherein \textcircled{A}, R¹, X, Y, R^{a}, R^{b}, R, m and L are as detailed herein.

In some embodiments, the compound is of Formula (XXVIII),

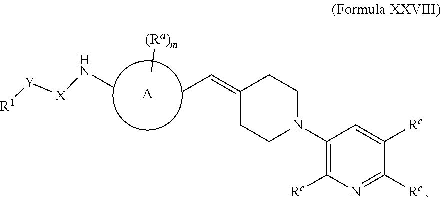

or a pharmaceutically acceptable salt thereof, wherein \textcircled{A}, R¹, X, Y, R^{a}, R^{c}, m and L are as detailed herein.

In some embodiments, the compound is of Formula (XXIX),

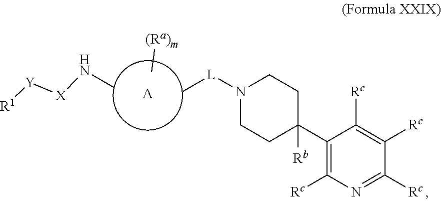

or a pharmaceutically acceptable salt thereof, wherein \textcircled{A}, R¹, X, Y, R^{a}, R^{b}, R^{c}, m and L are as detailed herein.

In some embodiments, the compound is of Formula (XXX′),

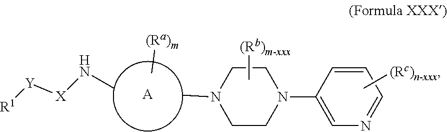

or a pharmaceutically acceptable salt thereof, wherein \textcircled{A}, R¹, X, Y, R^{a}, R^{b}, R^{c} and m are as detailed herein;
m-xxx is 0 to 8; and
n-xxx is 0 to 4.

In some embodiments, the compound of Formula (XXX′) is of Formula (XXX),

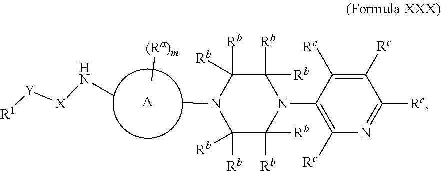

or a pharmaceutically acceptable salt thereof, wherein \textcircled{A}, R¹, X, Y, R^{a}, R^{b}, R^{c} and m are as detailed herein.

In some embodiments, the compound is of Formula (XXXI′),

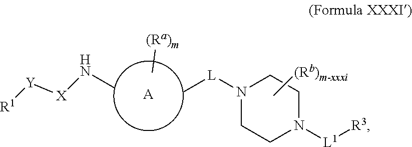

or a pharmaceutically acceptable salt thereof, wherein \textcircled{A}, R¹, R³, X, Y, R^{a}, R^{b}, m, L and L¹ are as detailed herein; and
m-xxxi is 0 to 8.

In some embodiments, the compound of Formula (XXXI′) is of Formula (XXXI),

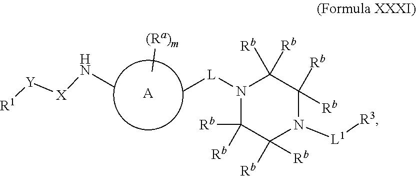

or a pharmaceutically acceptable salt thereof, wherein \textcircled{A}, R¹, R³, X, Y, R^{a}, R^{b}, m, L and L¹ are as detailed herein.

In some embodiments the compound is of Formula (XXXII′),

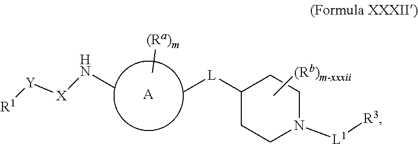

or a pharmaceutically acceptable salt thereof, wherein \textcircled{A}, R¹, R³, X, Y, R^{a}, R^{c}, m, L and L¹ are as detailed herein; and
m-xxxii is 0 to 9.

In some embodiments, the compound of Formula (XXXII′) is of Formula (XXXII),

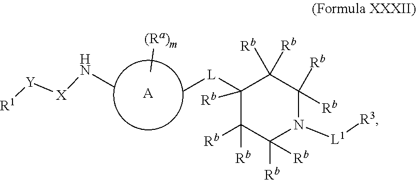

or a pharmaceutically acceptable salt thereof, wherein \textcircled{A}, R¹, R³, X, Y, R^{a}, R^{c}, m, L and L¹ are as detailed herein.

In some embodiments, the compound is of Formula (XXXIII′),

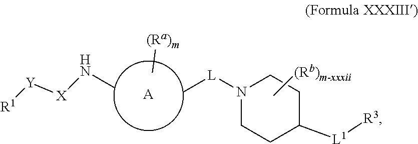

or a pharmaceutically acceptable salt thereof, wherein \textcircled{A}, R¹, R³, X, Y, R^{a}, R^{c}, m, L and L¹ are as detailed herein; and
m-xxxiii is 0 to 9.

In some embodiments, the compound of Formula (XXXIII′) is of Formula (XXXIII),

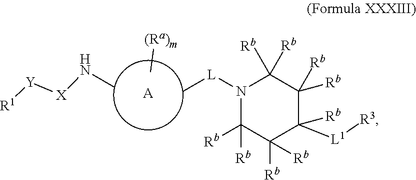

or a pharmaceutically acceptable salt thereof, wherein \textcircled{A}, R¹, R³, X, Y, R^{a}, R^{c}, m, L and L¹ are as detailed herein.

In some embodiments, the compound is of Formula (XXXIV),

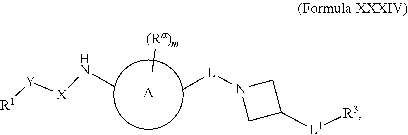

or a pharmaceutically acceptable salt thereof, wherein \textcircled{A}, R¹, R³, X, Y, R^{a}, m, L and L¹ are as detailed herein.

In some embodiments, the compound is of Formula (XXXV),

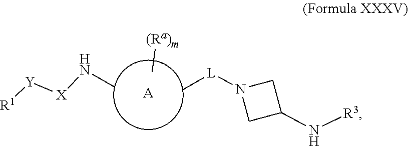

or a pharmaceutically acceptable salt thereof, wherein \textcircled{A}, R¹, R³, X, Y, R^{a}, m and L are as detailed herein.

In some embodiments, the compound is of Formula (XXXVI),

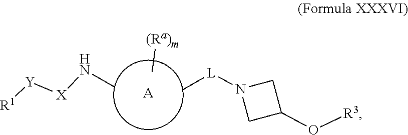

or a pharmaceutically acceptable salt thereof, wherein \textcircled{A}, R¹, R³, X, Y, R^{a}, m and L are as detailed herein.

In some embodiments, the compound is of Formula XXXVII,

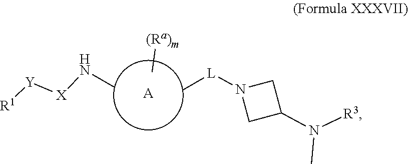

or a pharmaceutically acceptable salt thereof, wherein \textcircled{A}, R¹, R³, X, Y, R^{a}, m and L are as detailed herein.

In some embodiments, the compound is of Formula (XXXVIII),

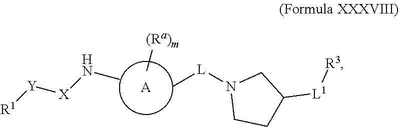

or a pharmaceutically acceptable salt thereof, wherein \textcircled{A}, R¹, R³, X, Y, R^{a}, m, L and L¹ are as detailed herein.

In some embodiments, the compound is of Formula XXXIX

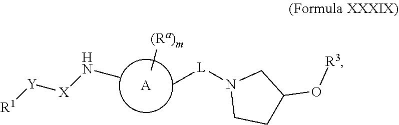

or a pharmaceutically acceptable salt thereof, wherein \textcircled{A}, R¹, R³, X, Y, R^{a}, m and L are as detailed herein.

In some embodiments, the compound is of Formula (XL),

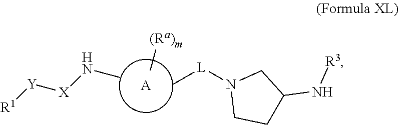

or a pharmaceutically acceptable salt thereof, wherein \textcircled{A}, R¹, R³, X, Y, R^{a}, m and L are as detailed herein.

In some embodiments, the compound is of Formula (XLI),

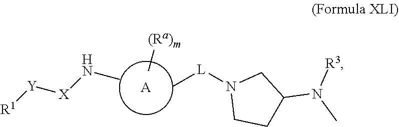

or a pharmaceutically acceptable salt thereof, wherein \textcircled{A}, R¹, R³, X, Y, R^{a}, m and L are as detailed herein.

In some embodiments, the compound is of Formula (XLII),

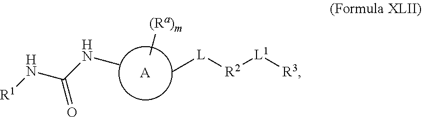

or a pharmaceutically acceptable salt thereof, wherein \textcircled{A}, R¹, R², R³, R^{a}, m, L, and L¹ are as detailed herein.

In some embodiments, the compound is of Formula (XLIII),

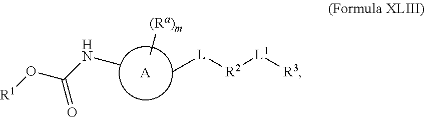

or a pharmaceutically acceptable salt thereof, wherein \textcircled{A}, R¹, R², R³, R^{a}, m, L, and L¹ are as detailed herein.

In some embodiments, the compound is of Formula (XLIV),

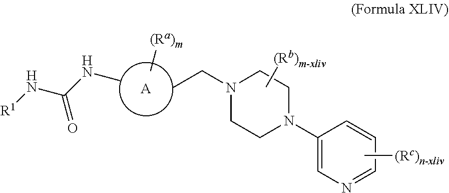

or a pharmaceutically acceptable salt thereof; wherein \textcircled{A}, R¹, R^{b}, and R^{c} are as detailed herein;
m-xliv is 0 to 8; and
n-xliv is 0 to 4.

It is understood that, specific values described herein are values for a compound of Formula (I) or any related formula where applicable, such as any of Formulae (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), (II-u), (II-v), (II-w), (II-x), (II-y), (II-z), (II-aa), (II-ab), (II-ac), (II-ad), (II-ae), (II-af), (II-ag), (II-ah), (II-ai), (II-aj), (II-ak), (II-al), (III-a), (III-b), (III-c), (III-d), (III-e), (III-f), (III-g), (III-h), (III-i), (III-j), (III-k), (III-l), (III-m), (III-n), (III-o), (III-p), (III-q), (III-r), (III-s), (III-t), (III-u), (III-v), (III-w), (III-x), (III-y), (III-z), (III-aa), (III-ab), (III-ac), (III-ad), (III-ae), (III-af), (III-ag), (III-ah), (III-ai), (III-aj), (III-ak), (III-al), (IV′), (IV), (V′), (V), (VW), (VI), (VII′), (VII), (VIII′), (VIII), (IX′), (IX), (X′), (X), (XI′), (XI), (XII), (XIII), (XIV), (XV), (XVI), (XVII), (XVIHI), (XIX), (XX), (XXI), (XXII), (XXHII′) (XXIII), (XXIV), (XXV), (XXVI), (XXVII), (XXVIII), (XXIX), (XXX′), (XXX), (XXXI′), (XXXI), (XXXII′), (XXXII), (XXXIII′), (XXXIH), (XXXIV), (XXXV), (XXXVI), (XXXVII), (XXXVIII), (XXXIX), (XL), (XLI), (XLII) (XLIII), and (XLIV) wherein \textcircled{A}, R¹, R², R³, X, Y, R¹, R^{b}, R^{c}, m, L and L¹ are as detailed herein. It is to be understood that two or more values may combined. Any of the groups described for any variable can be combined with any of the other groups described, where applicable, for any of the Formulae described herein. Thus, it is to be understood that any variable for a compound of Formula (I) or any related formula may be combined with any other variable for a compound of Formula (I) or any related formula the same as if each and every combination of variables were specifically and individually listed.

### Variables \textcircled{A}, m, and R^{a}

In some embodiments, \textcircled{A} is C₃₋₁₀cycloalkylene.

In some embodiments \textcircled{A} is cyclopentylene or cyclohexylene.

In some embodiments, \textcircled{A} is C₆₋₁₀ arylene.

In some embodiments, \textcircled{A} is phenylene.

In some embodiments, \textcircled{A} is 3- to 10-membered heterocycloalkylene.

In some embodiments, \textcircled{A} is pyrrolidinylene, imidazolidinylene, pyrazolidinylene, oazolidinylene, isoxazolidinylene, piperidinylene, oxanylene, diazinanylene, or morpholinylene.

In some embodiments, \textcircled{A} is pyrrolidinylene.

In some embodiments, \textcircled{A} is piperidinylene.

In some embodiments, \textcircled{A} is diazinanylene.

In some embodiments, \textcircled{A} is 5- to 10-membered heteroarylene.

In some embodiments, \textcircled{A} is pyrrolylene, furanylene, imidazolylene, pyrazolylene, oxazolylene, thiazolylene, isothiazolylene.

In some embodiments, \textcircled{A} is pyridinylene or diazinylene.

In some embodiments, \textcircled{A} is pyridinylene, pyimidinylene, pyridazinylene or triazinylene.

In some embodiments, m is 0.

In some embodiments, m is 1, 2, 3, 4, or 5.

In some embodiments, m is 1. In some embodiments, m is 2. In some embodiments, m is 3. In some embodiments, m is 4. In some embodiments, m is 5.

In some embodiments, at least one R^{a} is oxo, halogen, —CN, —OH, or —NH₂.

In some embodiments, at least one R¹ is oxo. In some embodiments, at least one R^{a} is halogen (e.g., F, Cl, or Br). In some embodiments, at least one R¹ is —CN. In some embodiments, at least one R¹ is —OH. In some embodiments, at least one R^{a} is —NH₂.

In some embodiments, at least one R^{a} is —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

In some embodiments, at least one R^{a} is —O—(C₁₋₆alkyl) optionally substituted with one or more oxo, deuterium, halogen, —CN, —OH, or —NH₂.

In some embodiments, at least one R¹ is —NH(C₁₋₆alkyl) optionally substituted with one or more oxo, deuterium, halogen, —CN, —OH, or —NH₂.

In some embodiments, at least one R^{a} is —N(C₁₋₆ alkyl)₂ optionally substituted with one or more oxo, deuterium, halogen, —CN, —OH, or —NH₂.

In some embodiments, at least one R¹ is C₁₋₆ alkyl (e.g., methyl, ethyl, propyl, butyl, pentyl, or hexyl) optionally substituted with one or more oxo, deuterium, halogen, —CN, —OH, or —NH₂.

In some embodiments, at least one R^{a} is C₂₋₆ alkenyl optionally substituted with one or more oxo, deuterium, halogen, —CN, —OH, or —NH₂.

In some embodiments, at least one R¹ is C₂₋₆ alkynyl optionally substituted with one or more oxo, deuterium, halogen, —CN, —OH, or —NH₂.

In some embodiments, at least one R^{a} is C₃₋₁₀ cycloalkyl optionally substituted with one or more oxo, deuterium, halogen, —CN, —OH, or —NH₂.

In some embodiments, at least one R¹ is C₆₋₁₀ aryl optionally substituted with one or more oxo, deuterium, halogen, —CN, —OH, or —NH₂.

In some embodiments, at least one R^{a} is 3- to 10-membered heterocycloalkyl optionally substituted with one or more oxo, deuterium, halogen, —CN, —OH, or —NH₂.

In some embodiments, at least one R^{a} is 5- to 10-membered heteroaryl optionally substituted with one or more oxo, deuterium, halogen, —CN, —OH, or —NH₂.

In some embodiments,

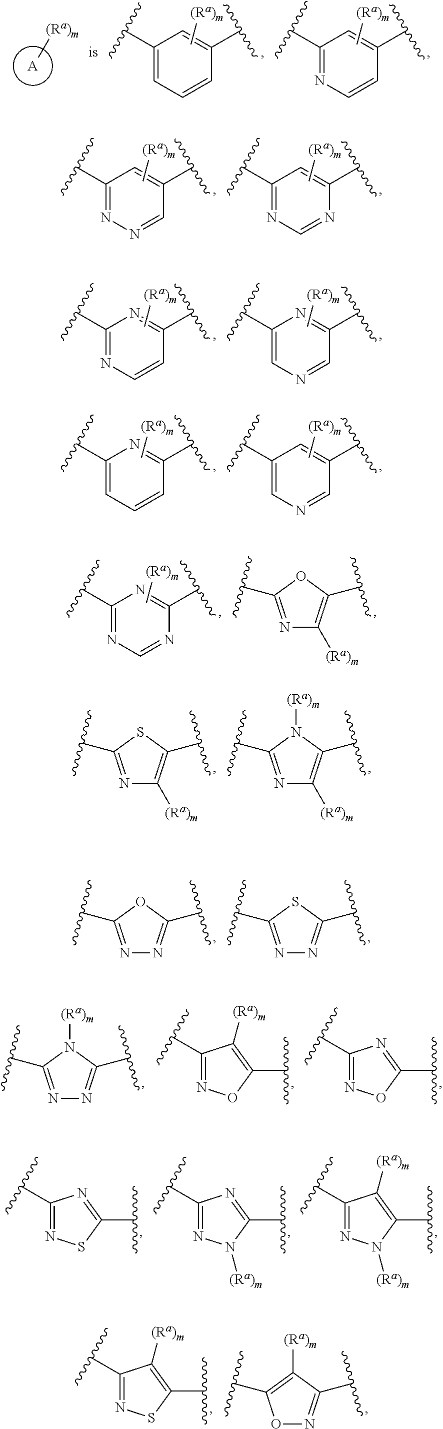

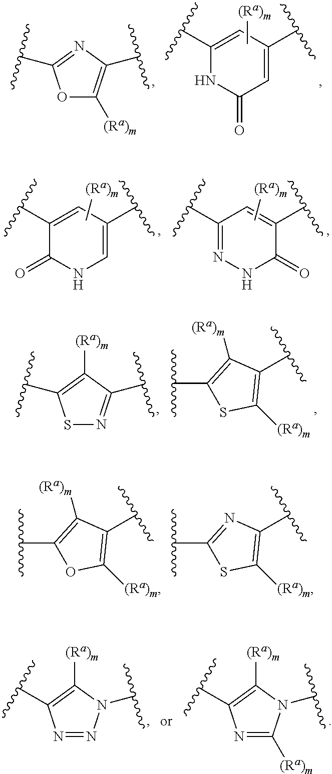

In some embodiments,

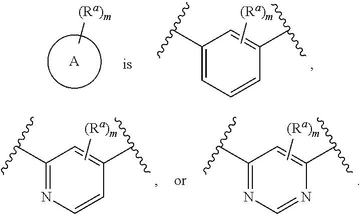

### Variables X Y, and R

In some embodiments, Y is a bond.

In some embodiments, Y is —O—, —S—, —NH—, or C₁₋₆ alkylene, wherein the C₁₋₆ alkylene is optionally substituted with one or more oxo, halogen, —CN, —OH, or —NH₂.

In some embodiments, Y is —O—, —S—, —NH—.

In some embodiments, Y is —O—. In some embodiments, Y is —S—. In some embodiments, Y is —NH—.

In some embodiments, Y is C₁₋₆ alkylene (e.g., methylene, ethylene, propylene, butylene, pentylene, or hexylene) optionally substituted with one or more oxo, halogen, —CN, —OH, or —NH₂.

In some embodiments, Y is C₁₋₆alkylene (e.g., methylene, ethylene, propylene, butylene, pentylene, or hexylene).

In some embodiments, Y is methylene, ethylene, or propylene.

In some embodiments, Y is methylene (e.g., —CH₂—). In some embodiments, L is ethylene (e.g., —CH₂CH₂—). In some embodiments, L is propylene (e.g., —CH₂CH₂CH₂—).

In some embodiments, Y is C₁₋₆alkylene (e.g., methylene, ethylene, propylene, butylene, pentylene, or hexylene) substituted with one or more oxo, halogen, —CN, —OH, or —NH₂.

In some embodiments, R¹ is H.

In some embodiments, R¹ is halogen, —CN, —OH, —NH₂, —O—(C₁₋₆alkyl), —NH(C₁₋₆ alkyl), or —N(C₁₋₆ alkyl)₂.

In some embodiments, R¹ is halogen (e.g., F, Cl, or Br).

In some embodiments, R¹ is —CN.

In some embodiments, R¹ is —OH.

In some embodiments, R¹ is —NH₂.

In some embodiments, R¹ is —O—(C₁₋₆ alkyl).

In some embodiments, R¹ is —NH(C₁₋₆ alkyl).

In some embodiments, R¹ is —N(C₁₋₆ alkyl)₂.

In some embodiments, R¹ is —N(C₁₋₆ alkyl)(C₂₋₆ alkyl).

In some embodiments, R¹ is —C₁₋₆ alkyl).

In some embodiments, R¹ is C₃₋₁₀cycloalkyl (e.g., cyclopropyl, cyclobutyl, cyclopentyl, or cyclohexyl) optionally substituted with one or more oxo, deuterium, halogen, —CN, —OH, or —NH₂.

In some embodiments, R¹ is cyclopropyl, cyclobutyl, cyclopentyl, or cyclohexyl, wherein the is cyclopropyl, cyclobutyl, cyclopentyl, or cyclohexyl is optionally substituted with one or more oxo, deuterium, halogen, —CN, —OH, or —NH₂.

In some embodiments, R¹ is cyclopropyl optionally substituted with one or more oxo, deuterium, halogen, —CN, —OH, or —NH₂.

In some embodiments, R¹ is cyclopropyl.

In some embodiments, R¹ is cyclobutyl optionally substituted with one or more oxo, deuterium, halogen, —CN, —OH, or —NH₂.

In some embodiments, R¹ is cyclobutyl.

In some embodiments, R¹ is C₆₋₁₀ aryl optionally substituted with one or more oxo, deuterium, halogen, —CN, —OH, or —NH₂.

In some embodiments, R¹ is 3- to 10-membered heterocycloalkyl optionally substituted with one or more oxo, deuterium, halogen, —CN, —OH, or —NH₂.

In some embodiments, R¹ is 5- to 10-membered heteroaryl optionally substituted with one or more oxo, deuterium, halogen, —CN, —OH, or —NH₂.

In some embodiments, R¹ is C₁₋₆ alkyl optionally substituted with one or more oxo, deuterium, halogen, —CN, —OH, or —NH₂.

Variables L, R², R^{b}, L¹, R³, R^{c}, and R^{bc}

In some embodiments, L is a bond.

In some embodiments, L is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;

In some embodiments, L is —O—, —S—, —S(═O)—, —S(═O)₂—, —NH—, —N(C₁₋₆ alkyl)-, —N(C₃₋₆ cycloalkyl)-, C₁₋₆alkylene, C₂₋₆alkenylene, or C₂₋₆alkynylene, wherein the —N(C₁₋₆ alkyl)-, —N(C₃-6 cycloalkyl)-, C₁₋₆ alkylene, C₂₋₆ alkenylene, or C₂₋₆ alkynylene is optionally substituted with one or more oxo, deuterium, halogen, —CN, —OH, or —NH₂.

In some embodiments, L is —O—, —S—, —S(═O)—, —S(═O)₂—, or —NH—.

In some embodiments, L is —O—. In some embodiments, L is —S—. In some embodiments, L is —S(═O)—. In some embodiments, L is —S(═O)₂—. In some embodiments, L is —NH—.

In some embodiments, L is —N(C₁₋₆ alkyl)-, —N(C₃₋₆ cycloalkyl)-, C₁₋₆ alkylene, C₂₋₆ alkenylene, or C₂₋₆ alkynylene, wherein the —N(C₁₋₆ alkyl)-, —N(C₃₋₆ cycloalkyl)-, C₁₋₆alkylene, C₂₋₆ alkenylene, or C₂₋₆ alkynylene is optionally substituted with one or more oxo, deuterium, halogen, —CN, —OH, or —NH₂.

In some embodiments, L is —N(C₁₋₆ alkyl)- optionally substituted with one or more oxo, deuterium, halogen, —CN, —OH, or —NH₂.

In some embodiments, L is —N(C₃₋₆ cycloalkyl)- optionally substituted with one or more oxo, deuterium, halogen, —CN, —OH, or —NH₂.

In some embodiments, L is —N(3- to 6-membered heterocycloalkyl)- optionally substituted with one or more oxo, deuterium, halogen, —CN, —OH, or —NH₂.

In some embodiments, L is C₁₋₆alkylene (e.g., methylene, ethylene, propylene, butylene, pentylene, or hexylene) optionally substituted with one or more oxo, deuterium, halogen, —CN, —OH, or —NH₂.

In some embodiments, L is C₁₋₆alkylene (e.g., methylene, ethylene, propylene, butylene, pentylene, or hexylene).

In some embodiments, L is methylene, ethylene, or propylene.

In some embodiments, L is methylene (e.g., —CH₂—). In some embodiments, L is ethylene (e.g., —CH₂CH₂—). In some embodiments, L is propylene (e.g., —CH₂CH₂CH₂—).

In some embodiments, L is C₁₋₆ alkylene (e.g., methylene, ethylene, propylene, butylene, pentylene, or hexylene) substituted with one or more oxo, deuterium, halogen, —CN, —OH, or —NH₂.

In some embodiments, L is C₂₋₆ alkenylene optionally substituted with one or more oxo, deuterium, halogen, —CN, —OH, or —NH₂.

In some embodiments, L is C₂₋₆ alkynylene optionally substituted with one or more oxo, deuterium, halogen, —CN, —OH, or —NH₂.

In some embodiments, Lis C₁₋₆ cycloalkylene optionally substituted with one or more oxo, deuterium, halogen, —CN, —OH, or —NH₂.

In some embodiments, L is C₃₋₆ heterocycloalkylene optionally substituted with one or more oxo, deuterium, halogen, —CN, —OH, or —NH₂.

In some embodiments, L is bond, —CH₂—, —CH₂—CH₂—, —CH₂—CH₂—CH₂—, —C(O)—, —O—, —S—, —O—, —NH—, —N(CH₃)—,

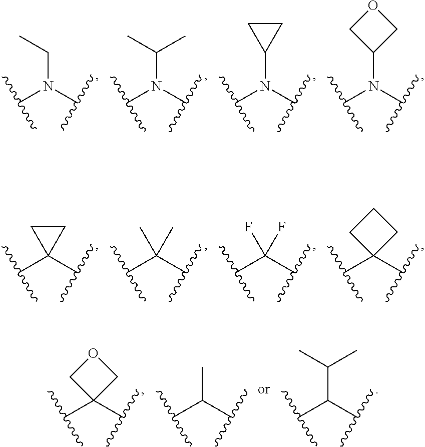

In some embodiments, L is —N(CH₃)—,

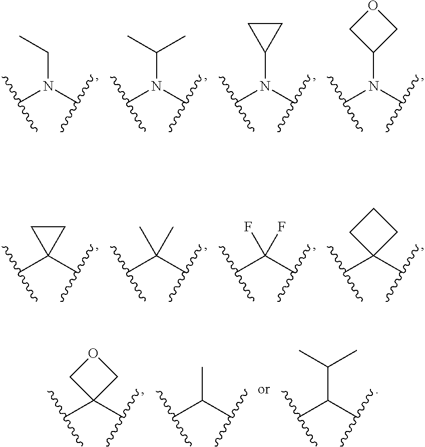

In some embodiments, L is C₁ or C₂ alkylene optionally substituted with oxo.

In some embodiments, L is

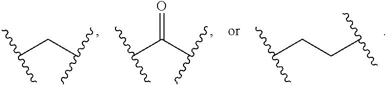

In some embodiments, L is

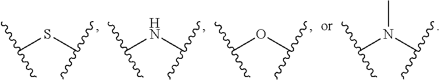

In some embodiments, R² is C₃₋₁₀ cycloalkylene optionally substituted with one or more R^{b}.

In some embodiments, R² is

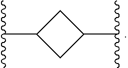 [SMILES: *C1CC(*)C1]

In some embodiments, R² is C₆₋₁₀ arylene (e.g., phenylene) optionally substituted with one or more R^{b}.

In some embodiments, R² is 3- to 10-membered heterocycloalkylene optionally substituted with one or more R^{b}.

In some embodiments, R² is 3- to 10-membered heterocycloalkylene comprising at least one N atom (e.g., aziridinylene, azetidinylene, pyrrolidinylene, or piperidinylene), wherein the 3- to 10-membered heterocycloalkylene is optionally substituted with one or more R^{b}.

In some embodiments, R² is aziridinylene, azetidinylene, pyrrolidinylene, or piperidinylene, wherein the aziridinylene, azetidinylene, pyrrolidinylene, or piperidinylene is optionally substituted with one or more R^{b}.

In some embodiments, R² is aziridinylene optionally substituted with one or more R^{b}.

In some embodiments, R² is aziridinylene.

In some embodiments, R² is azetidinylene optionally substituted with one or more R^{b}.

In some embodiments, R² is azetidinylene.

In some embodiments, R² is pyrrolidinylene

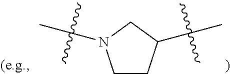 [SMILES: *C1CCN(*)C1]

optionally substituted with one or more R^{b}.

In some embodiments, R² is pyrrolidinylene

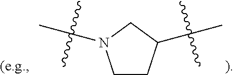 [SMILES: *C1CCN(*)C1]

In some embodiments, R² is piperidinylene optionally substituted with one or more R^{b}.

In some embodiments, R² is piperidinylene.

In some embodiments, R² is 3- to 10-membered heterocycloalkylene comprising two N atoms (e.g., diazinanylene), wherein the 3- to 10-membered heterocycloalkylene is optionally substituted with one or more R^{b}.

In some embodiments, R² is 3- to 10-membered heterocycloalkylene comprising two N atoms (e.g., diazinanylene), wherein the 3- to 10-membered heterocycloalkylene is optionally substituted with one or more R^{b}.

In some embodiments, R² is diazinanylene

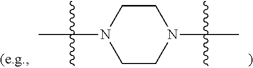 [SMILES: *N1CCN(*)CC1]

optionally substituted with one or more R^{b}.

In some embodiments, R² is diazinanylene

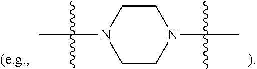 [SMILES: *N1CCN(*)CC1]

In some embodiments, R² is diazinanylene 3,6-diazabicyclo[3.1.1]heptenylene

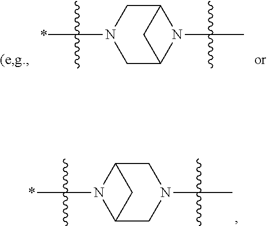

in which * denotes attachment to L) optionally substituted with one or more R^{b}.

In some embodiments, R² is diazinanylene 3,6-diazabicyclo[3.1.1]heptenylene

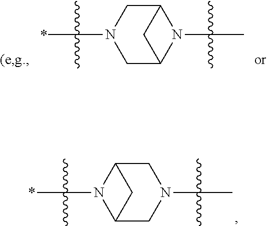

in which * denotes attachment to L).

In some embodiments, R² is 5- to 10-membered heteroarylene optionally substituted with one or more R^{b}.

In some embodiments, R² is

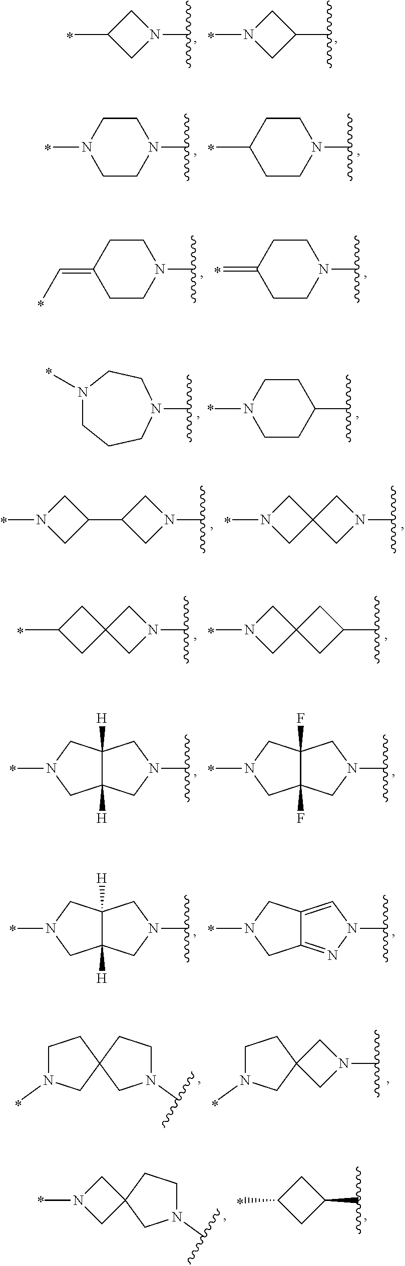

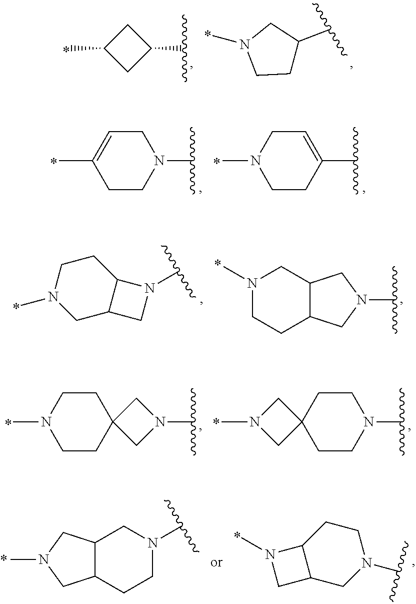

each of which is optionally substituted with one or more R^{b} and wherein * and [symbol] denotes the point of attachment to L and L¹ respectively.

In some embodiments, R² is

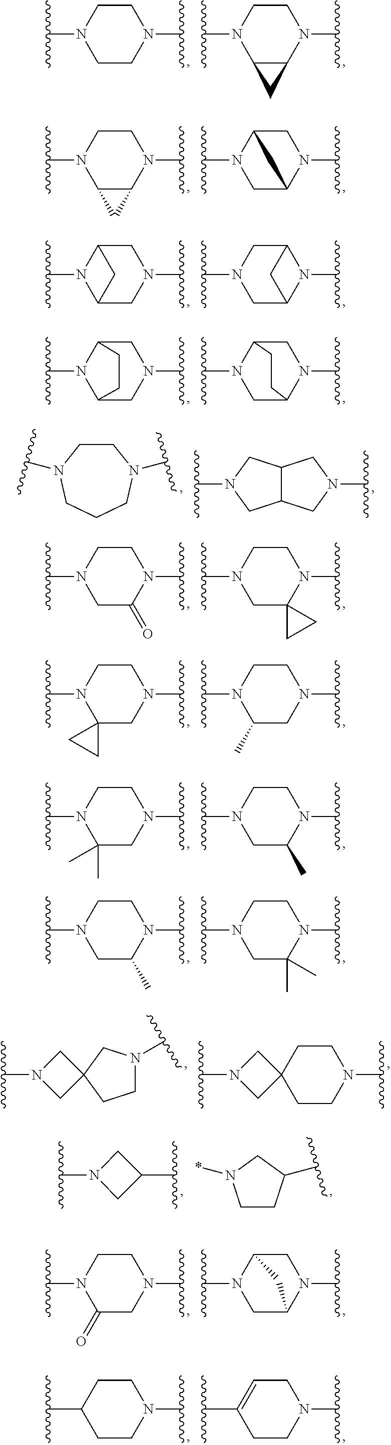

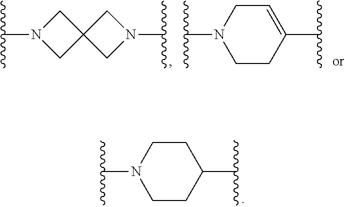

In some embodiments, R² is

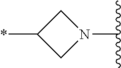 [SMILES: *C1CN(*)C1]

optionally substituted with one or more R^{b}. In some embodiments, R² is

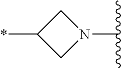 [SMILES: *C1CN(*)C1]

optionally substituted with one or more R^{b}. In some embodiments, R² is

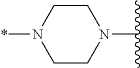 [SMILES: *N1CCN(*)CC1]

optionally substituted with one or more R^{b}. In some embodiments, R² is

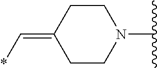 [SMILES: *C=C1CCN(*)CC1]

optionally substituted with one or more R^{b}. In some embodiments, R² is

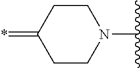 [SMILES: *N1CCC(=*)CC1]

optionally substituted with one or more R^{b}. In some embodiments, R² is

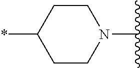 [SMILES: *C1CCN(*)CC1]

optionally substituted with one or more R^{b}. In some embodiments, R² is

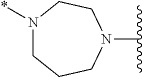 [SMILES: *N1CCCN(*)CC1]

optionally substituted with one or more R^{b}. In some embodiments, R² is

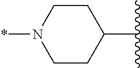 [SMILES: *C1CCN(*)CC1]

optionally substituted with one or more R^{b}. In some embodiments, R² is

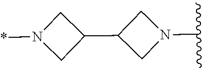 [SMILES: *N1CC(C2CN(*)C2)C1]

optionally substituted with one or more R^{b}. In some embodiments, R² is

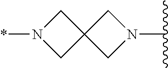 [SMILES: *N1CC2(C1)CN(*)C2]

optionally substituted with one or more R^{b}. In some embodiments, R² is

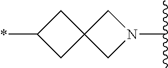 [SMILES: *C1CC2(C1)CN(*)C2]

optionally substituted with one or more R^{b}. In some embodiments, R² is

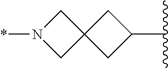 [SMILES: *C1CC2(C1)CN(*)C2]

optionally substituted with one or more R^{b}. In some embodiments, R² is

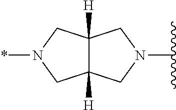 [SMILES: *N1C[C@H]2CN(*)C[C@H]2C1]

optionally substituted with one or more R^{b}. In some embodiments, R² is

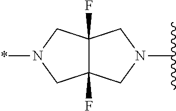 [SMILES: *N1C[C@]2(F)CN(*)C[C@]2(F)C1]

optionally substituted with one or more R^{b}. In some embodiments, R² is

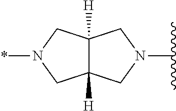 [SMILES: *N1C[C@H]2CN(*)C[C@@H]2C1]

optionally substituted with one or more R^{b}. In some embodiments, R² is

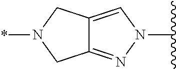 [SMILES: *N1Cc2cn(*)nc2C1]

optionally substituted with one or more R^{b}. In some embodiments, R² is

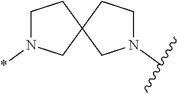 [SMILES: *N1CCC2(CCN(*)C2)C1]

optionally substituted with one or more R^{b}. In some embodiments, R² is

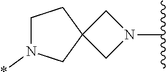 [SMILES: *N1CCC2(C1)CN(*)C2]

optionally substituted with one or more R^{b}. In some embodiments, R² is

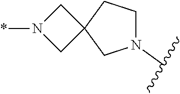 [SMILES: *N1CCC2(C1)CN(*)C2]

optionally substituted with one or more R^{b}. In some embodiments, R² is

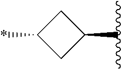 [SMILES: *[C@H]1C[C@H](*)C1]

optionally substituted with one or more R^{b}. In some R² is

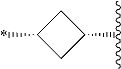 [SMILES: *[C@H]1C[C@@H](*)C1]

optionally substituted with one or more R^{b}. In some embodiments, R² is

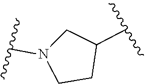 [SMILES: *C1CCN(*)C1]

optionally substituted with one or more R^{b}. In some embodiments, R² is

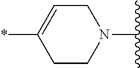 [SMILES: *C1=CCN(*)CC1]

optionally substituted with one or more R^{b}. In some embodiments, R² is

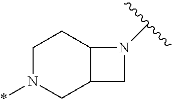 [SMILES: *N1CCC2C(C1)CN2*]

optionally substituted with one or more R^{b}. In some embodiments, R² is

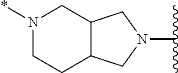 [SMILES: *N1CCC2CN(*)CC2C1]

optionally substituted with one or more R^{b}. In some embodiments, R² is

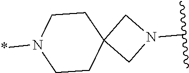 [SMILES: *N1CCC2(CC1)CN(*)C2]

optionally substituted with one or more R^{b}. In some embodiments, R² is

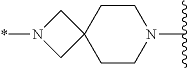 [SMILES: *N1CCC2(CC1)CN(*)C2]

optionally substituted with one or more R^{b}. In some embodiments, R² is

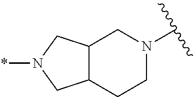 [SMILES: *N1CCC2CN(*)CC2C1]

optionally substituted with one or more R^{b}. In some embodiments, R² is

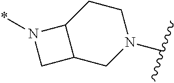 [SMILES: *N1CCC2C(C1)CN2*]

optionally substituted with one or more R^{b}.

In some embodiments, at least one R^{b} is oxo, halogen, —CN, —OH, or —NH₂.

In some embodiments, at least one R^{b} is oxo. In some embodiments, at least one R^{b} is halogen (e.g., F, Cl, or Br). In some embodiments, at least one R^{b} is —CN. In some embodiments, at least one R^{b} is —OH. In some embodiments, at least one R^{b} is —NH₂.

In some embodiments, at least one R^{b} is —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

In some embodiments, at least one R^{b} is —O—(C₁₋₆ alkyl) optionally substituted with one or more oxo, deuterium, halogen, —CN, —OH, or —NH₂.

In some embodiments, at least one R^{b} is —NH(C₁₋₆ alkyl) optionally substituted with one or more oxo, deuterium, halogen, —CN, —OH, or —NH₂.

In some embodiments, at least one R^{b} is —N(C₁₋₆ alkyl)₂ optionally substituted with one or more oxo, deuterium, halogen, —CN, —OH, or —NH₂.

In some embodiments, at least one R^{b} is C₁₋₆ alkyl (e.g., methyl, ethyl, propyl, butyl, pentyl, or hexyl) optionally substituted with one or more oxo, deuterium, halogen, —CN, —OH, or —NH₂.

In some embodiments, at least one R^{a} is C₂₋₆ alkenyl optionally substituted with one or more oxo, deuterium, halogen, —CN, —OH, or —NH₂.

In some embodiments, at least one R^{b} is C₂₋₆ alkynyl optionally substituted with one or more oxo, deuterium, halogen, —CN, —OH, or —NH₂.

In some embodiments, at least one R^{b} is C₃₋₁₀ cycloalkyl optionally substituted with one or more oxo, deuterium, halogen, —CN, —OH, or —NH₂.

In some embodiments, at least one R^{b} is C₆₋₁₀ aryl optionally substituted with one or more oxo, deuterium, halogen, —CN, —OH, or —NH₂.

In some embodiments, at least one R^{b} is 3- to 10-membered heterocycloalkyl optionally substituted with one or more oxo, deuterium, halogen, —CN, —OH, or —NH₂.

In some embodiments, at least one R^{b} is 5- to 10-membered heteroaryl optionally substituted with one or more oxo, deuterium, halogen, —CN, —OH, or —NH₂.

In some embodiments, IV is a bond.

In some embodiments, L¹ is —O—, —S—, —S(═O)—, —S(═O)₂—, —NH—, —N(C₁₋₆ alkyl)-, —N(C₃₋₆ cycloalkyl)-, C₁₋₆alkylene, C₂₋₆alkenylene, or C₂₋₆alkynylene, wherein the —N(C₁₋₆ alkyl)-, —N(C₃₋₆ cycloalkyl)-, C₁₋₆ alkylene, C₂₋₆ alkenylene, or C₂₋₆ alkynylene is optionally substituted with one or more oxo, deuterium, halogen, —CN, —OH, or —NH₂.

In some embodiments, L is —O—, —S—, —S(═O)—, —S(═O)₂—, or —NH—.

In some embodiments, L¹ is —O—. In some embodiments, L¹ is —S—. In some embodiments, L¹ is —S(═O)—. In some embodiments, L¹ is —S(═O)₂—. In some embodiments, L¹ is —NH—.

In some embodiments, L¹ is —N(C₁₋₆ alkyl)-, —N(C₃₋₆ cycloalkyl)-, C₁₋₆ alkylene, C₂₋₆ alkenylene, or C₂₋₆alkynylene, wherein the —N(C₁₋₆ alkyl)-, —N(C₃₋₆ cycloalkyl)-, C₁₋₆alkylene, C₂₋₆ alkenylene, or C₂₋₆ alkynylene is optionally substituted with one or more oxo, deuterium, halogen, —CN, —OH or —NH₂.

In some embodiments, L¹ is —N(C₁₋₆ alkyl)- optionally substituted with one or more oxo, deuterium, halogen, —CN, —OH, or —NH₂.

In some embodiments, L¹ is —N(C₃₋₆ cycloalkyl)- optionally substituted with one or more oxo, deuterium, halogen, —CN, —OH, or —NH₂.

In some embodiments, L¹ is —N(3- to 6-membered heterocycloalkyl)- optionally substituted with one or more oxo, deuterium, halogen, —CN, —OH, or —NH₂.

In some embodiments, L¹ is C₁₋₆ alkylene (e.g., methylene, ethylene, propylene, butylene, pentylene, or hexylene) optionally substituted with one or more oxo, deuterium, halogen, —CN, —OH, or —NH₂.

In some embodiments, L¹ is C₁₋₆alkylene (e.g., methylene, ethylene, propylene, butylene, pentylene, or hexylene).

In some embodiments, L¹ is methylene, ethylene, or propylene.

In some embodiments, L¹ is methylene (e.g., —CH₂—). In some embodiments, L¹ is ethylene (e.g., —CH₂CH₂—). In some embodiments, L¹ is propylene (e.g., —CH₂CH₂CH₂—).

In some embodiments, L¹ is C₁₋₆alkylene (e.g., methylene, ethylene, propylene, butylene, pentylene, or hexylene) substituted with one or more oxo, deuterium, halogen, —CN, —OH, or —NH₂.

In some embodiments, L¹ is C₂₋₆ alkenylene optionally substituted with one or more oxo, halogen, deuterium, —CN, —OH, or —NH₂.

In some embodiments, L¹ is C₂₋₆alkynylene optionally substituted with one or more oxo, deuterium, halogen, —CN, —OH, or —NH₂.

In some embodiments, L¹ is C₃₋₆ cycloalkylene optionally substituted with one or more oxo, deuterium, halogen, —CN, —OH, or —NH₂.

In some embodiments, L¹ is C₃₋₆ heterocycloalkylene optionally substituted with one or more oxo, deuterium, halogen, —CN, —OH, or —NH₂.

In some embodiments, L¹ is —N(CH₃)—,

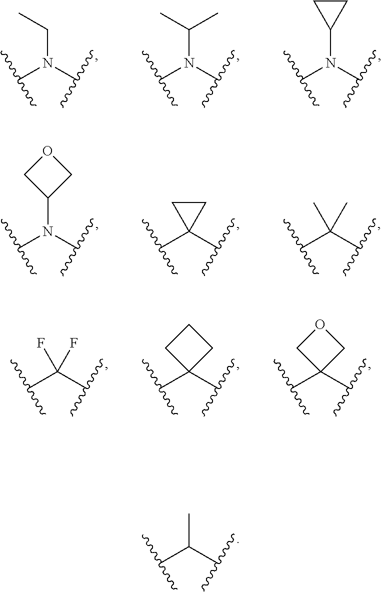

In some embodiments, L¹ is

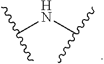 [SMILES: *N*]

In some embodiments, L¹ is —O—.

In some embodiments, L¹ is

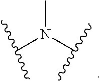 [SMILES: *N(*)C]

In some embodiments, R³ is C₃₋₁₀ cycloalkyl optionally substituted with one or more R^{c}.

In some embodiments, R³ is C₃₋₁₀ cycloalkyl substituted with one or more halogen, —C(═O)—H, —C(═O)—NH₂, —NH—C(═O)—H, —NH—(C═O)—NH₂, —C(═O)—CN, —NH—C(═O)—CN, —C(═O)—NH—CN, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), or C₁₋₆ alkyl.

In some embodiments, R³ is C₃₋₁₀ cycloalkyl substituted with one or more halogen, —C(═O)—NH₂, —C(═O)—NH—CN, —C(═O)—NH—(C₁₋₆ alkyl), or C₁₋₆ alkyl.

In some embodiments, R³ is C₃₋₁₀ cycloalkyl substituted with one or more —C(═O)—NH₂, —C(═O)—NH—CN, or —C(═O)—NH—(C₁₋₆ alkyl).

In some embodiments, R³ is C₆₋₁₀ aryl (e.g., phenyl) optionally substituted with one or more R¹.

In some embodiments, R³ is C₆₋₁₀ aryl (e.g., phenyl).

In some embodiments, R³ is C₆₋₁₀ aryl (e.g., phenyl) substituted with one or more R^{c}.

In some embodiments, R³ is C₆₋₁₀ aryl (e.g., phenyl) substituted with one or more halogen, —C(═O)—H, —C(═O)—NH₂, —NH—C(═O)—H, —NH—(C═O)—NH₂, —C(═O)—CN, —NH—C(═O)—CN, —C(═O)—NH—CN, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), or C₁₋₆ alkyl.

In some embodiments, R³ is C₆₋₁₀ aryl (e.g., phenyl) substituted with one or more halogen, —C(═O)—NH₂, —C(═O)—NH—CN, —C(═O)—NH—(C₁₋₆ alkyl), or C₁₋₆ alkyl.

In some embodiments, R³ is C₆₋₁₀ aryl (e.g., phenyl) substituted with one or more —C(═O)—NH₂, —C(═O)—NH—CN, or —C(═O)—NH—(C₁₋₆ alkyl).

In some embodiments, R³ is 3- to 10-membered heterocycloalkyl optionally substituted with one or more R^{c}.

In some embodiments, R³ is 5- to 10-membered heteroaryl (e.g., pyridinyl) optionally substituted with one or more R^{c}.

In some embodiments, R³ is pyridinyl

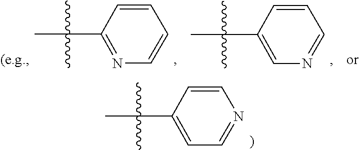

optionally substituted with one or more R^{c}.

In some embodiments, R³ is 5- to 10-membered heteroaryl.

In some embodiments, R³ is pyridinyl

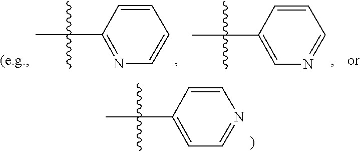

In some embodiments, R³ is 5- to 10-membered heteroaryl (e.g., pyridinyl) substituted with one or more R^{c}.

In some embodiments, R¹ is 5- to 10-membered heteroaryl comprising at least one N atom (e.g., pyridinyl), wherein the heteroaryl is substituted with one or more R^{c}.

In some embodiments, R³ is 5- to 10-membered heteroaryl comprising at least one N atom (e.g., pyridinyl), wherein the heteroaryl is substituted with one or more halogen, CN, —C(═O)—H, —C(═O)—NH₂, —NH—C(═O)—H, —NH—(C═O)—NH₂, —C(═O)—CN, —NH—C(═O)—CN, —C(═O)—NH—CN, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), —NH—(C═O)—O—(C₁₋₆ alkyl) or C₁₋₆ alkyl.

In some embodiments, R³ is 5- to 10-membered heteroaryl comprising at least one N atom (e.g., pyridinyl), wherein the heteroaryl is substituted with one or more halogen, —C(═O)—NH₂, —C(═O)—NH—CN, —C(═O)—NH—(C₁₋₆ alkyl), or C₁₋₆ alkyl.

In some embodiments, R³ is pyridinyl

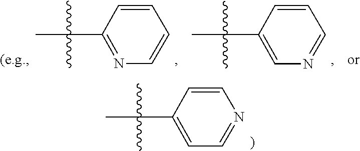

substituted with one or more R^{c}.

In some embodiments, R³ is

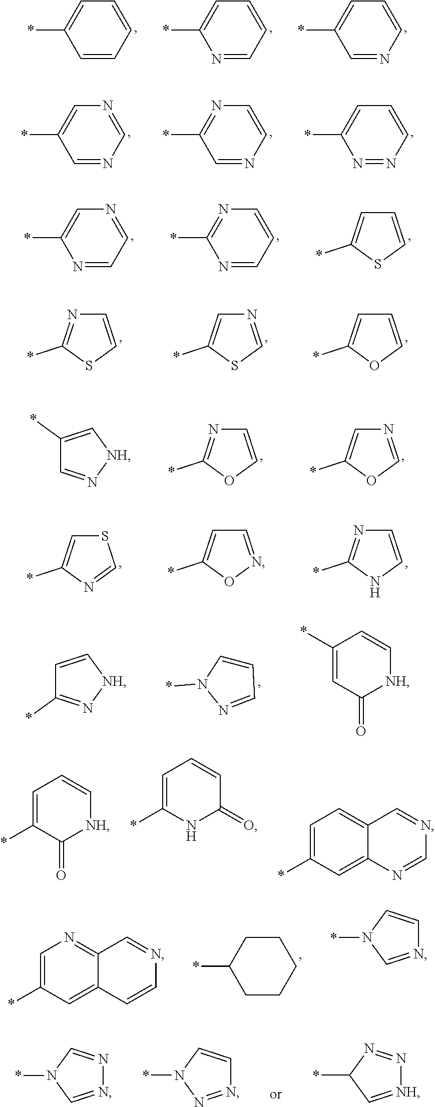

each of which is optionally substituted with one or more R^{c} and * denotes point of attachment to L¹. In some embodiments, R³ is

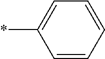 [SMILES: *c1ccccc1]

optionally substituted with one or more R^{c}. In some embodiments, R³ is

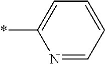 [SMILES: *c1ccccn1]

optionally substituted with one or more R^{c}. In some embodiments, R³ is

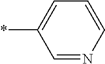 [SMILES: *c1cccnc1]

optionally substituted with one or more R^{c}. In some embodiments, R³ is

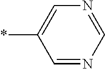 [SMILES: *c1cncnc1]

optionally substituted with one or more R^{c}. In some embodiments, R³ is

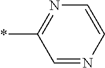 [SMILES: *c1cnccn1]

optionally substituted with one or more R^{c}. In some embodiments, R³ is

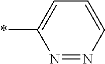 [SMILES: *c1cccnn1]

optionally substituted with one or more R^{c}. In some embodiments, R³ is

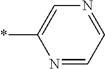 [SMILES: *c1cnccn1]

optionally substituted with one or more R^{c}. In some embodiments, R³ is

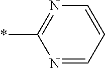 [SMILES: *c1ncccn1]

optionally substituted with one or more R^{c}. In some embodiments, R³ is

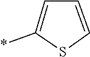 [SMILES: *c1cccs1]

optionally substituted with one or more R^{c}. In some embodiments, R³ is

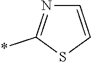 [SMILES: *c1nccs1]

optionally substituted with one or more R^{c}. In some embodiments, R³ is

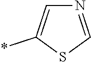 [SMILES: *c1cncs1]

optionally substituted with one or more R^{c}. In some embodiments, R³ is

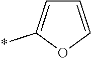 [SMILES: *c1ccco1]

optionally substituted with one or more R^{c}. In some embodiments, R³ is

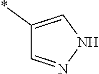 [SMILES: *c1cn[nH]c1]

optionally substituted with one or more R^{c}. In some embodiments, R³ is

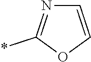 [SMILES: *c1ncco1]

optionally substituted with one or more R^{c}. In some embodiments, R³ is

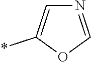 [SMILES: *c1cnco1]

optionally substituted with one or more R^{c}. In some embodiments, R³ is

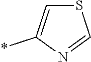 [SMILES: *c1cscn1]

optionally substituted one or more R^{c}. In some embodiment, R³ is

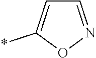 [SMILES: *c1ccno1]

optionally substituted with one or more R^{c}. In some embodiments, R³ is

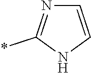 [SMILES: *c1ncc[nH]1]

optionally substituted with one or more R^{c}. In some embodiments, R³ is

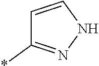 [SMILES: *c1cc[nH]n1]

optionally substituted with one or more R^{c}. In some embodiments, R³ is

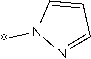 [SMILES: *n1cccn1]

optionally substituted with one more R^{c}. In some embodiments, R³ is

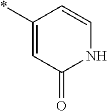 [SMILES: *c1cc[nH]c(=O)c1]

optionally substituted with one or more R^{c}. In some embodiments, R³ is

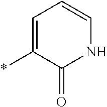 [SMILES: *c1ccc[nH]c1=O]

optionally substituted with one or more R^{c}. In some embodiments, R³ is

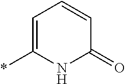 [SMILES: *c1cccc(=O)[nH]1]

optionally substituted with one or more R^{c}. In some embodiments, R³ is

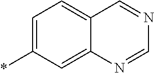 [SMILES: *c1ccc2cncnc2c1]

optionally substituted with one one or more R^{c}. In some embodiments, R³ is

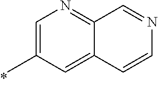

optionally substituted with one or more R^{c}. In some embodiments, R³ is

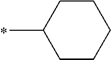

optionally substituted with one or more R^{c}. In some embodiments, R³ is

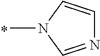

optionally substituted with one or more R^{c}. In some embodiments, R³ is

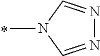

optionally substituted with one or more R^{c}. In some embodiments, R³ is

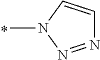

optionally substituted with one or more R^{c}. In some embodiments, R³ is

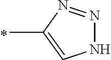

optionally substituted with one or more R^{c}.

In some embodiments, R³ is

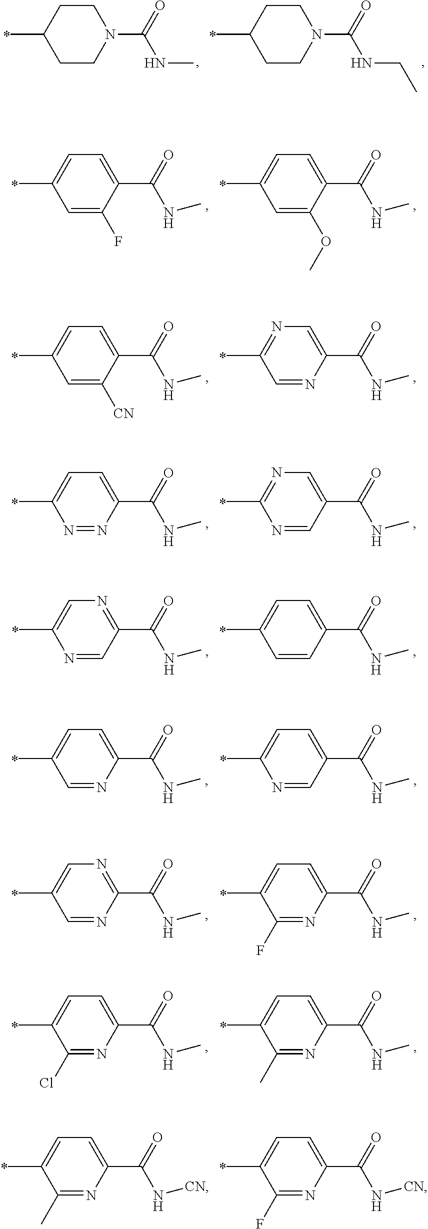

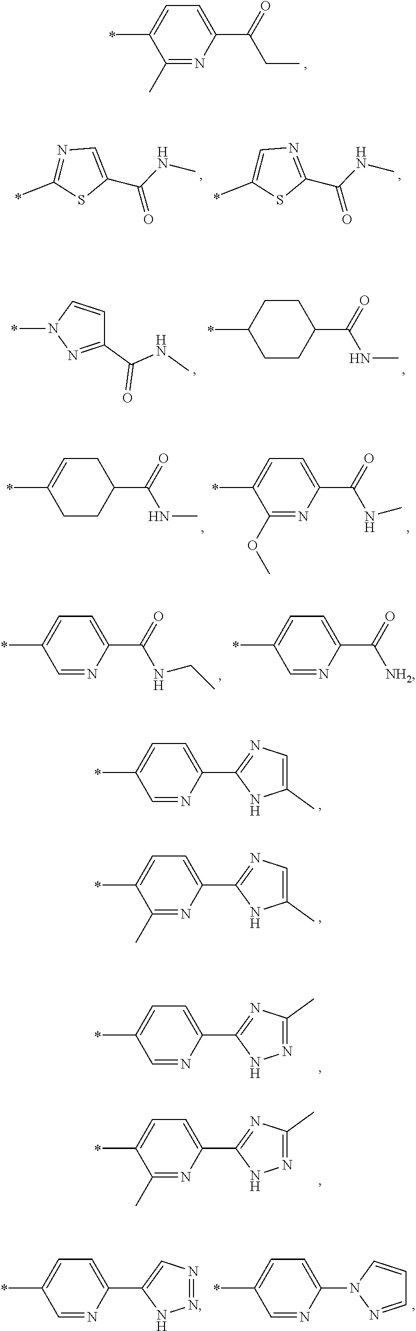

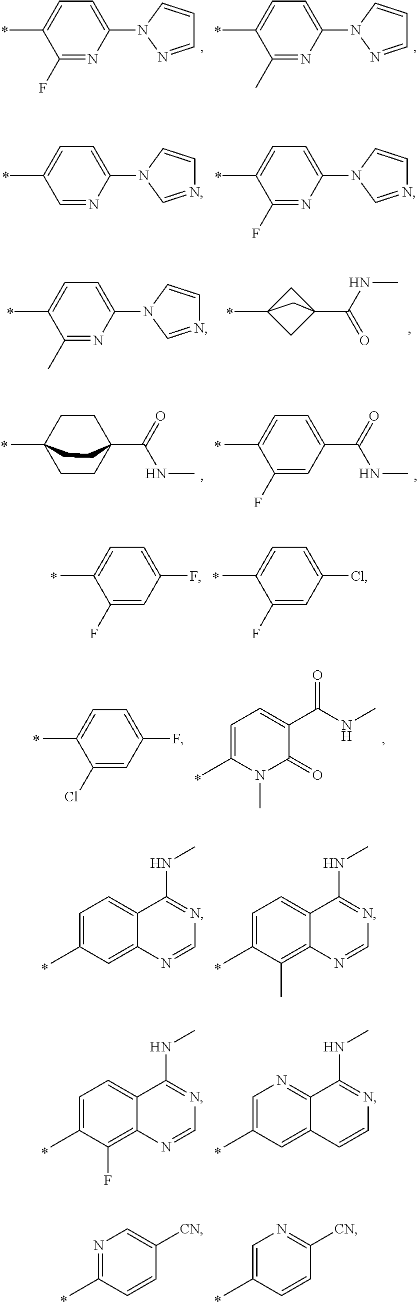

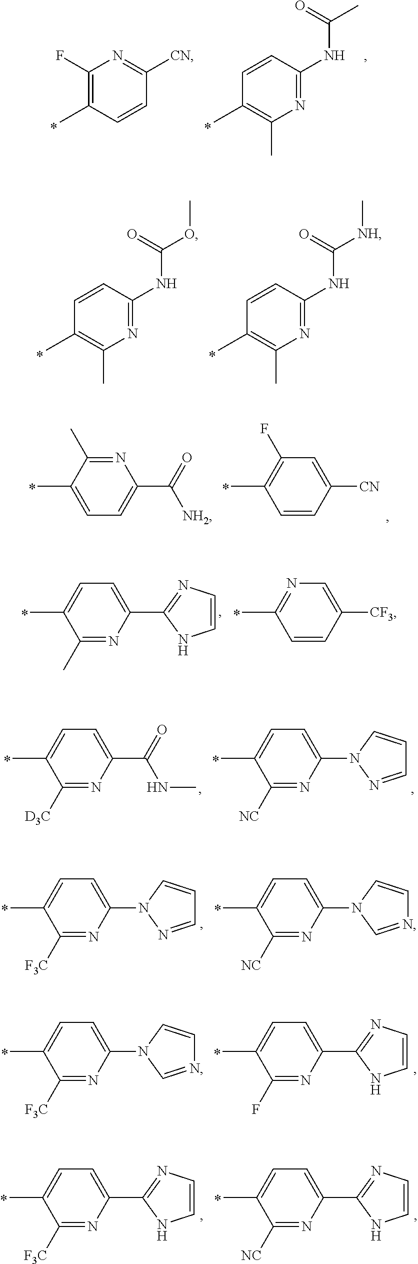

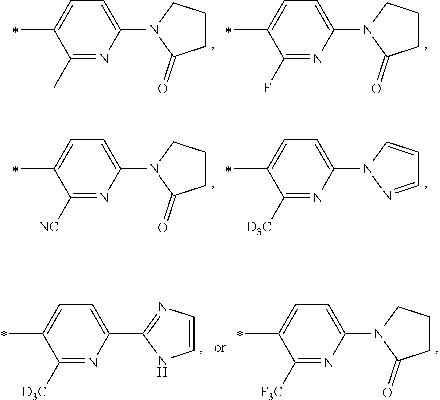

wherein * denotes the point of attachment to L¹.

In some embodiments, R³ is

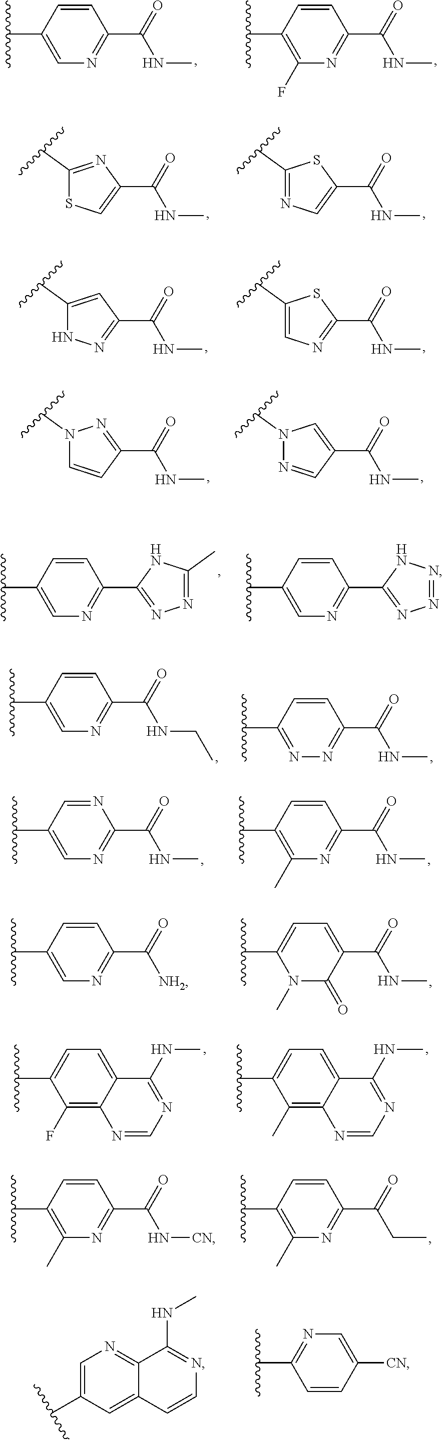

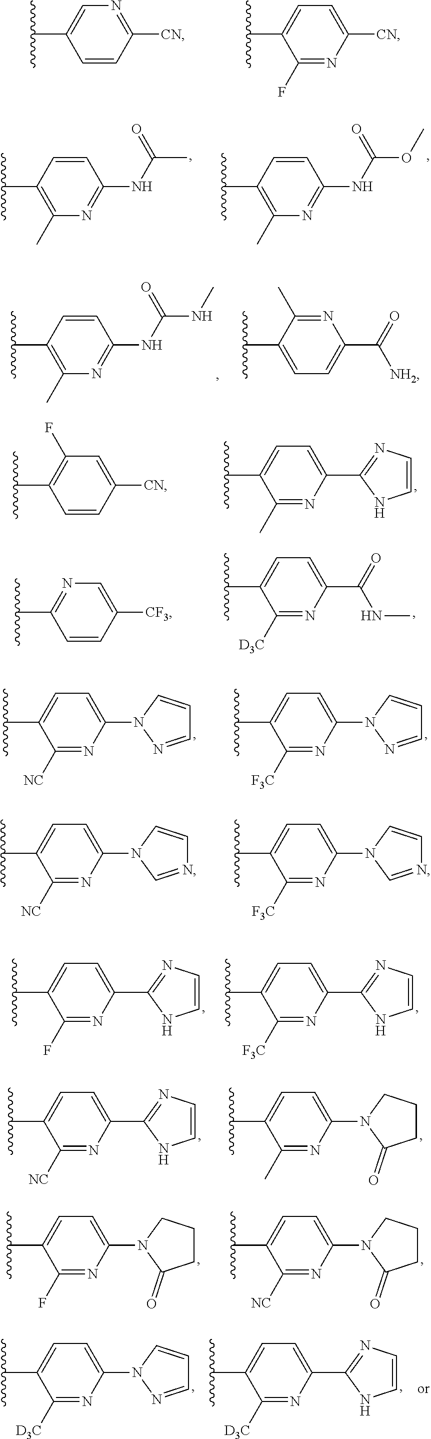

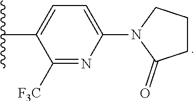 [SMILES: *c1ccc(N2CCCC2=O)nc1C(F)(F)F]

In some embodiments, R³ is

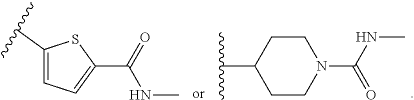

In some embodiments, R³ is

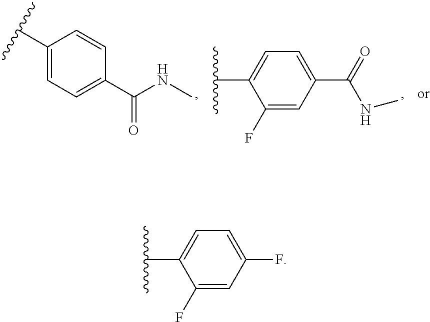

In some embodiments, at least one R^{c} is oxo, halogen, —CN, —OH, —NH₂, —C(═O)—H, —C(═O)—NH₂, —NH—C(═O)—H, —NH—(C═O)—NH₂, —C(═O)—CN, —NH—C(═O)—CN, or —C(═O)—NH—CN.

In some embodiments, at least one R^{c} is oxo.

In some embodiments, at least one R^{c} is halogen (e.g., F, Cl, or Br).

In some embodiments, at least one R^{c} is —CN. In some embodiments, at least one R^{c} is —OH. In some embodiments, at least one R^{c} is —NH₂. In some embodiments, at least one R¹ is —C(═O)—H. In some embodiments, at least one R^{c} is —C(═O)—NH₂. In some embodiments, at least one R^{c} is —NH—C(═O)—H. In some embodiments, at least one R^{c} is —NH—(C═O)—NH₂. In some embodiments, at least one R^{c} is —C(═O)—CN. In some embodiments, at least one R¹ is —NH—C(═O)—CN. In some embodiments, at least one R^{c} is —C(═O)—NH—CN.

In some embodiments, at least one R^{c} is —O—(C₁₋₆alkyl), —NH(C₁₋₆alkyl), —N(C₁₋₆alkyl)₂, —C(═O)—(C₁₋₆ alkyl), —C(═O)—NH—(C₁₋₆alkyl), —NH—C(═O)—(C₁₋₆alkyl), —NH—(C═O)—NH—(C₁₋₆ alkyl), —NH—(C═O)—O—(C₁₋₆ alkyl), —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), —NH—(C═O)—O—(C₁₋₆ alkyl), —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

In some embodiments, at least one R^{c} is —O—(C₁₋₆ alkyl) optionally substituted with one or more oxo, deuterium, halogen, —CN, —OH, or —NH₂. In some embodiments, at least one R^{c} is —NH(C₁₋₆ alkyl) optionally substituted with one or more oxo, deuterium, halogen, —CN, —OH, or —NH₂. In some embodiments, at least one R^{c} is —N(C₁₋₆ alkyl)₂ optionally substituted with one or more oxo, deuterium, halogen, —CN, —OH, or —NH₂.

In some embodiments, at least one R^{c} is —C(═O)—(C₁₋₆ alkyl) (e.g., —C(═O)—CH₃) optionally substituted with one or more oxo, deuterium, halogen, —CN, —OH, or —NH₂.

In some embodiments, at least one R^{c} is —C(═O)—(C₁₋₆ alkyl) (e.g., —C(═O)—CH₃).

In some embodiments, at least one R^{c} is —C(═O)—CH₃.

In some embodiments, at least one R^{c} is —C(═O)—NH—(C₁₋₆ alkyl) (e.g., —C(═O)—NH—CH₃) optionally substituted with one or more oxo, deuterium, halogen, —CN, —OH, or —NH₂.

In some embodiments, at least one R^{c} is —C(═O)—NH—(C₁₋₆ alkyl) (e.g., —C(═O)—NH—CH₃).

In some embodiments, at least one R¹ is —C(═O)—NH—CH₃.

In some embodiments, at least one R^{c} is —NH—C(═O)—(C₁₋₆ alkyl) (e.g., —NH—C(═O)—CH₃) optionally substituted with one or more oxo, deuterium, halogen, —CN, —OH, or —NH₂.

In some embodiments, at least one R^{c} is —NH—C(═O)—(C₁₋₆ alkyl) (e.g., —NH—C(═O)—CH₃).

In some embodiments, at least one R^{c} —NH—C(═O)—CH₃.

In some embodiments, at least one R^{c} is —NH—C(═O)—(C₁₋₆ alkyl) (e.g., —NH—C(═O)—CH₃) optionally substituted with one or more oxo, deuterium, halogen, —CN, —OH, or —NH₂.

In some embodiments, at least one R^{c} is —NH—C(═O)—(C₁₋₆ alkyl) (e.g., —NH—C(═O)—CH₃).

In some embodiments, at least one R^{c} is —NH—C(═O)—CH₃.

In some embodiments, at least one R^{c} is —NH—(C═O)—NH—(C₁₋₆ alkyl) (e.g., —NH—(C═O)—NH—CH₃) optionally substituted with one or more oxo, deuterium, halogen, —CN, —OH, or —NH₂.

In some embodiments, at least one R^{c} is —NH—(C═O)—NH—(C₁₋₆ alkyl) (e.g., —NH—(C═O)—NH—CH₃).

In some embodiments, at least one R^{c} is —NH—(C═O)—O—(C₁₋₆ alkyl) (e.g., —NH—(C═O)—O—CH₃) optionally substituted with one or more oxo, deuterium, halogen, —CN, —OH, or —NH₂.

In some embodiments, at least one R^{c} is —NH—(C═O)—O—(C₁₋₆ alkyl) (e.g., —NH—(C═O)—O—CH₃).

In some embodiments, at least one R^{c} is —NH—(C═O)—O—CH₃.

In some embodiments, at least one R^{c} is —NH—(C═O)—NH—CH₃.

In some embodiments, at least one R^{c} is —C(═O)—NH—(C₃₋₁₀ cycloalkyl) optionally substituted with one or more oxo, deuterium, halogen, —CN, —OH, or —NH₂.

In some embodiments, at least one R^{c} is —C(═O)—NH—(C₃₋₁₀ cycloalkyl).

In some embodiments, at least one R^{c} is —C(═O)—NH-(3- to 10-membered heterocycloalkyl) optionally substituted with one or more oxo, deuterium, halogen, —CN, —OH, or —NH₂.

In some embodiments, at least one R^{c} is —C(═O)—NH-(3- to 10-membered heterocycloalkyl).

In some embodiments, at least one R^{c} is C₁₋₆ alkyl optionally substituted with one or more oxo, deuterium, halogen, —CN, —OH, or —NH₂. In some embodiments, at least one R^{c} is C₂₋₆ alkenyl optionally substituted with one or more oxo, deuterium, halogen, —CN, —OH, or —NH₂. In some embodiments, at least one R^{c} is C₂₋₆ alkynyl.

In some embodiments, at least one R^{c} is C₃₋₁₀ cycloalkyl optionally substituted with one or more oxo, deuterium, halogen, —CN, —OH, or —NH₂. In some embodiments, at least one R^{c} is C₆₋₁₀ aryl optionally substituted with one or more oxo, deuterium, halogen, —CN, —OH, or —NH₂. In some embodiments, at least one R^{c} is 3- to 10-membered heterocycloalkyl optionally substituted with one or more oxo, deuterium, halogen, —CN, —OH, or —NH₂. In some embodiments, at least one R^{c} is 5- to 10-membered heteroaryl (e.g., imidazolyl) optionally substituted with one or more oxo, deuterium, halogen, —CN, —OH, or —NH₂.

In some embodiment, one R^{b} and one R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀cycloalkyl, C₆₋₁₀ aryl, 3- to 10-membered heterocycloalkyl, or 5- to 10-membered heteroaryl is optionally substituted with one or more R^{c}.

In some embodiment, one R^{b} and one R^{c}, together with the intervening atoms they are attached to, form C₃₋₁₀ cycloalkyl optionally substituted with one or more R^{b}.

In some embodiment, one R^{b} and one R^{c}, together with the intervening atoms they are attached to, form C₆₋₁₀ aryl optionally substituted with one or more R¹.

In some embodiment, one R^{b} and one R^{c}, together with the intervening atoms they are attached to, form 3- to 10-membered heterocycloalkyl optionally substituted with one or more R^{bc}.

In some embodiment, one R^{b} and one R^{c}, together with the intervening atoms they are attached to, form 5- to 10-membered heteroaryl optionally substituted with one or more R¹.

In some embodiments, at least one R^{bc} is oxo, deuterium, halogen, —CN, —OH, or —NH₂.

In some embodiments, at least one R^{bc} is oxo. In some embodiments, at least one R¹ is deuterium. In some embodiments, at least one R^{bc} is halogen (e.g., F, Cl, or Br). In some embodiments, at least one R¹ is —CN. In some embodiments, at least one R^{b} is —OH. In some embodiments, at least one R¹ is —NH₂.

In some embodiments, at least one R^{b} is —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

In some embodiments, at least one R¹ is —O—(C₁₋₆alkyl) optionally substituted with one or more oxo, deuterium, halogen, —CN, —OH, or —NH₂.

In some embodiments, at least one R¹ is —NH(C₁₋₆ alkyl) optionally substituted with one or more oxo, deuterium, halogen, —CN, —OH, or —NH₂.

In some embodiments, at least one R¹ is —N(C₁₋₆ alkyl)₂ optionally substituted with one or more oxo, deuterium, halogen, —CN, —OH, or —NH₂.

In some embodiments, at least one R¹ is C₁₋₆ alkyl (e.g., methyl, ethyl, propyl, butyl, pentyl, or hexyl) optionally substituted with one or more oxo, deuterium, halogen, —CN, —OH, or —NH₂.

In some embodiments, at least one R^{bc} is C₂₋₆ alkenyl optionally substituted with one or more oxo, deuterium, halogen, —CN, —OH, or —NH₂.

In some embodiments, at least one R¹ is C₂₋₆ alkynyl optionally substituted with one or more oxo, deuterium, halogen, —CN, —OH, or —NH₂.

In some embodiments, at least one R^{bc} is C₃₋₁₀ cycloalkyl optionally substituted with one or more oxo, deuterium, halogen, —CN, —OH, or —NH₂.

In some embodiments, at least one R^{k} is C₆₋₁₀ aryl optionally substituted with one or more oxo, deuterium, halogen, —CN, —OH, or —NH₂.

In some embodiments, at least one R^{bc} is 3- to 10-membered heterocycloalkyl optionally substituted with one or more oxo, deuterium, halogen, —CN, —OH, or —NH₂.

In some embodiments, at least one R^{bc} is 5- to 10-membered heteroaryl optionally substituted with one or more oxo, deuterium, halogen, —CN, —OH, or —NH₂.

### Exemplary Compounds

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the compounds described in Table 1.

TABLE 1

| No. | Structure | IUPAC Name |
|---|---|---|
| 1 | 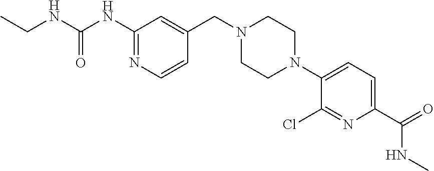 | 6-chloro-5-(4-((2-(3-ethylureido)pyridin- 4-yl)methyl)piperazin-1-yl)-N- methylpicolinamide |
| 2 | 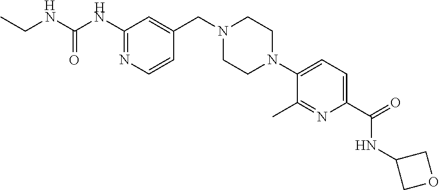 | 5-(4-((2-(3-ethylureido)pyridin-4- yl)methyl)piperazin-1-yl)-6-methyl-N- (oxetan-3-yl)picolinamide |
| 3 | 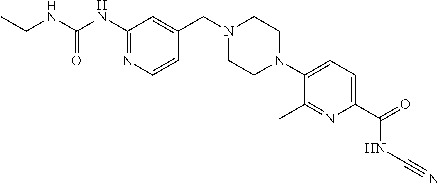 | N-cyano-5-(4-((2-(3-ethylurcido)pyridin- 4-yl)methyl)piperazin-1-yl)-6- methylpicolinamide |
| 4 | 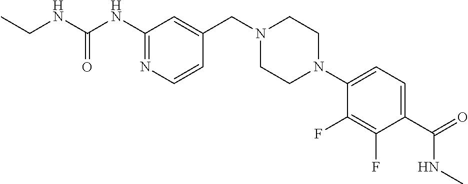 [SMILES: CCNC(=O)Nc1cc(CN2CCN(c3ccc(C(=O)NC)c(F)c3F)CC2)ccn1] | 4-(4-((2-(3-ethylureido)pyridin-4- yl)methyl)piperazin-1-yl)-2,3-difluoro-N- methylbenzamide |
| 5 | 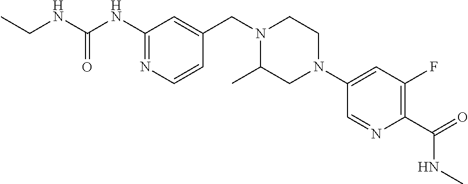 [SMILES: CCNC(=O)Nc1cc(CN2CCN(c3cnc(C(=O)NC)c(F)c3)CC2C)ccn1] | 5-(4-((2-(3-ethylureido)pyridin-4- yl)methyl)-3-methylpiperazin-1-yl)-3- fluoro-N-methylpicolinamide |
| 6 | 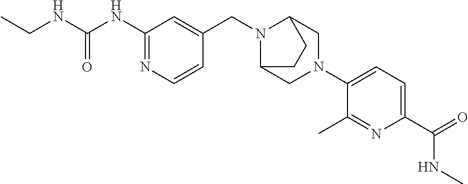 [SMILES: CCNC(=O)Nc1cc(CN2C3CCC2CN(c2ccc(C(=O)NC)nc2C)C3)ccn1] | 5-(8-((2-(3-ethylureido)pyridin-4- yl)methyl)-3,8-diazabicyclo[3.2.1]octan- 3-yl)-N,6-dimethylpicolinamide |
| 7 | 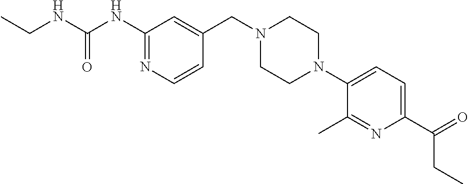 [SMILES: CCNC(=O)Nc1cc(CN2CCN(c3ccc(C(=O)CC)nc3C)CC2)ccn1] | 1-ethyl-3-(4-((4-(2-methyl-6- propionylpyridin-3-yl)piperazin-1- yl)methyl)pyridin-2-yl)urea 5-(5-((2-(3-ethylureido)pyridin-4- yl)methyl)-2,5-diazabicyclo[4.1.0]heptan- 2-yl)-3-fluoro-N-methylpicolinamide |
| 8 | 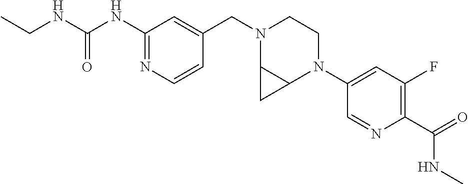 [SMILES: CCNC(=O)Nc1cc(CN2CCN(c3cnc(C(=O)NC)c(F)c3)C3CC32)ccn1] | 5-(5-((2-(3-ethylureido)pyridin-4- yl)methyl)-2,5-diazabicyclo[4.1.0]heptan- 2-yl)-3-fluoro-N-methylpicolinamide |
| 9 | 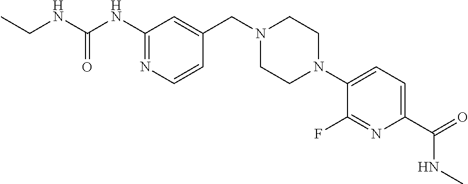 [SMILES: CCNC(=O)Nc1cc(CN2CCN(c3ccc(C(=O)NC)nc3F)CC2)ccn1] | 5-(4-((2-(3-ethylureido)pyridin-4- yl)methyl)piperazin-1-yl)-6-fluoro-N- methylpicolinamide |
| 10 | 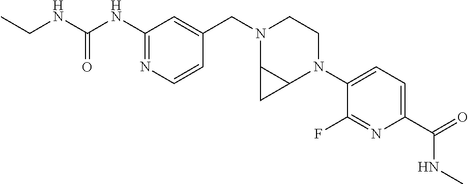 [SMILES: CCNC(=O)Nc1cc(CN2CCN(c3ccc(C(=O)NC)nc3F)C3CC32)ccn1] | 5-(5-((2-(3-ethylureido)pyridin-4- yl)methyl)-2,5-diazabicyclo[4.1.0]heptan- 2-yl)-6-fluoro-N-methylpicolinamide |
| 11 | 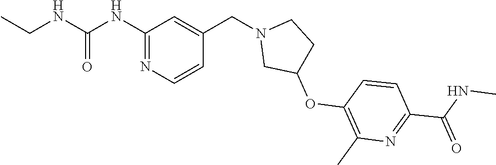 [SMILES: CCNC(=O)Nc1cc(CN2CCC(Oc3ccc(C(=O)NC)nc3C)C2)ccn1] | 5-((1-((2-(3-ethylureido)pyridin-4- yl)methyl)pyrrolidin-3-yl)oxy)-N,6- dimethylpicolinamide |
| 12 | 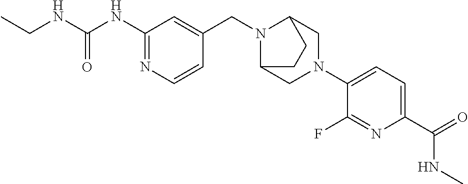 [SMILES: CCNC(=O)Nc1cc(CN2C3CCC2CN(c2ccc(C(=O)NC)nc2F)C3)ccn1] | 5-(8-((2-(3-ethylureido)pyridin-4- yl)methyl)-3,8-diazabicyclo[3.2.1]octan- 3-yl)-6-fluoro-N-methylpicolinamide |
| 13 | 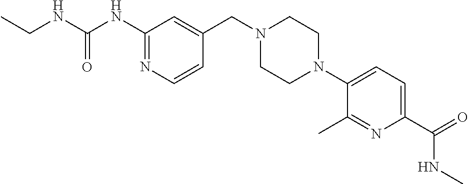 [SMILES: CCNC(=O)Nc1cc(CN2CCN(c3ccc(C(=O)NC)nc3C)CC2)ccn1] | 5-(4-((2-(3-ethylureido)pyridin-4- yl)methyl)piperazin-1-yl)-N,6- dimethylpicolinamide |
| 14 | 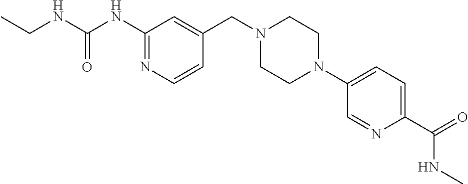 [SMILES: CCNC(=O)Nc1cc(CN2CCN(c3ccc(C(=O)NC)nc3)CC2)ccn1] | 5-(4-((2-(3-ethylureido)pyridin-4- yl)methyl)piperazin-1-yl)-N- methylpicolinamide |
| 15 | 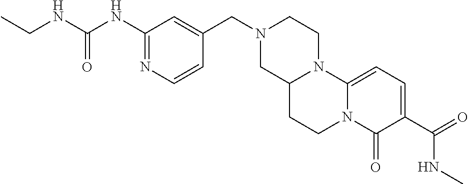 [SMILES: CCNC(=O)Nc1cc(CN2CCN3c4ccc(C(=O)NC)c(=O)n4CCC3C2)ccn1] | 3-((2-(3-ethylureido)pyridin-4- yl)methyl)-N-methyl-8-oxo-2,3,4,4a,5,6- hexahydro-1H,8H-pyrazino[1,2- c]pyrido[1,2-a]pyrimidine-9-carboxamide |
| 16 | 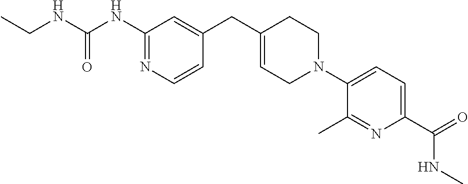 [SMILES: CCNC(=O)Nc1cc(CC2=CCN(c3ccc(C(=O)NC)nc3C)CC2)ccn1] | 4-((2-(3-ethylureido)pyridin-4- yl)methyl)-N,2′-dimethyl-3,6-dihydro- 2H-[1,3′-bipyridine]-6′-carboxamide |
| 17 | 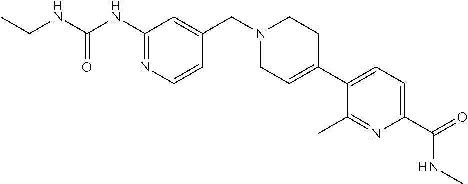 [SMILES: CCNC(=O)Nc1cc(CN2CC=C(c3ccc(C(=O)NC)nc3C)CC2)ccn1] | 1′-((2-(3-ethylureido)pyridin-4- yl)methyl)-N,2-dimethyl-1′,2′,3′,6′- tetrahydro-[3,4′-bipyridine]-6- carboxamide |
| 18 | 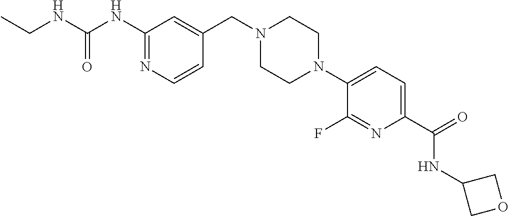 [SMILES: CCNC(=O)Nc1cc(CN2CCN(c3ccc(C(=O)NC4COC4)nc3F)CC2)ccn1] | 5-(4-((2-(3-ethylureido)pyridin-4- yl)methyl)piperazin-1-yl)-6-fluoro-N- (oxetan-3-yl)picolinamide |
| 19 | 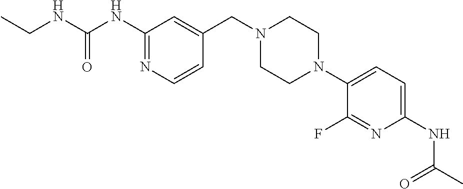 [SMILES: CCNC(=O)Nc1cc(CN2CCN(c3ccc(NC(C)=O)nc3F)CC2)ccn1] | N-(5-(4-((2-(3-ethylureido)pyridin-4- yl)methyl)piperazin-1-yl)-6- fluoropyridin-2-yl)acetamide |
| 20 | 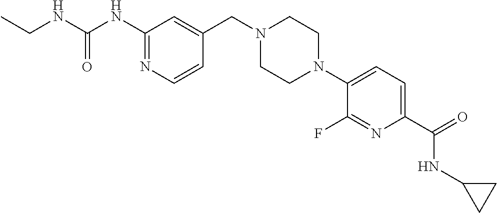 [SMILES: CCNC(=O)Nc1cc(CN2CCN(c3ccc(C(=O)NC4CC4)nc3F)CC2)ccn1] | N-cyclopropyl-5-(4-((2-(3- ethylureido)pyridin-4- yl)methyl)piperazin-1-yl)-6- fluoropicolinamide |
| 21 | 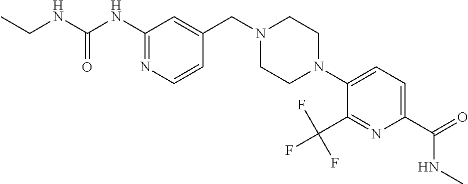 [SMILES: CCNC(=O)Nc1cc(CN2CCN(c3ccc(C(=O)NC)nc3C(F)(F)F)CC2)ccn1] | 5-(4-((2-(3-ethylureido)pyridin-4- yl)methyl)piperazin-1-yl)-N-methyl-6- (trifluoromethyl)picolinamide |
| 22 | 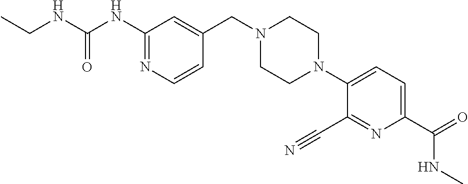 [SMILES: CCNC(=O)Nc1cc(CN2CCN(c3ccc(C(=O)NC)nc3C#N)CC2)ccn1] | 6-cyano-5-(4-((2-(3-ethylureido)pyridin- 4-yl)methyl)piperazin-1-yl)-N- methylpicolinamide |
| 23 | 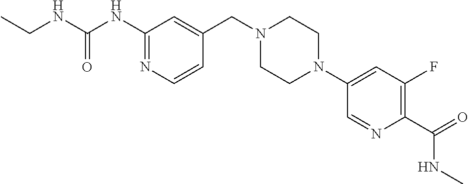 [SMILES: CCNC(=O)Nc1cc(CN2CCN(c3cnc(C(=O)NC)c(F)c3)CC2)ccn1] | 5-(4-((2-(3-ethylureido)pyridin-4- yl)methyl)piperazin-1-yl)-3-fluoro-N- methylpicolinamide |
| 24 | 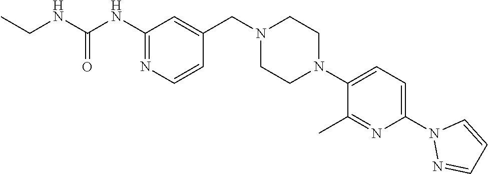 [SMILES: CCNC(=O)Nc1cc(CN2CCN(c3ccc(-n4cccn4)nc3C)CC2)ccn1] | 1-ethyl-3-(4-((4-(2-methyl-6-(1H- pyrazol-1-yl)pyridin-3-yl)piperazin-1- yl)methyl)pyridin-2-yl)urea |
| 25 | 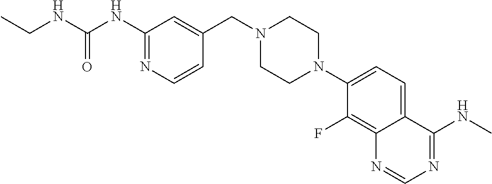 [SMILES: CCNC(=O)Nc1cc(CN2CCN(c3ccc4c(NC)ncnc4c3F)CC2)ccn1] | 1-ethyl-3-(4-((4-(8-fluoro-4- (methylamino)quinazolin-7-yl)piperazin- 1-yl)methyl)pyridin-2-yl)urea |
| 26 | 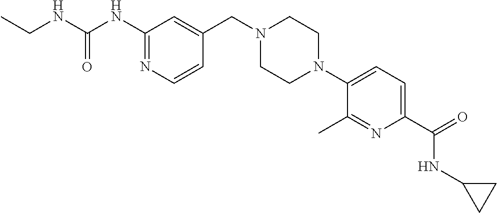 [SMILES: CCNC(=O)Nc1cc(CN2CCN(c3ccc(C(=O)NC4CC4)nc3C)CC2)ccn1] | N-cyclopropyl-5-(4-((2-(3- ethylureido)pyridin-4- yl)methyl)piperazin-1-yl)-6- methylpicolinamide |
| 27 | 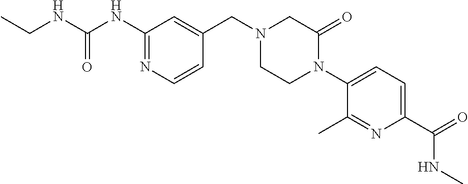 [SMILES: CCNC(=O)Nc1cc(CN2CCN(c3ccc(C(=O)NC)nc3C)C(=O)C2)ccn1] | 5-(4-((2-(3-ethylureido)pyridin-4- yl)methyl)-2-oxopiperazin-1-yl)-N,6- dimethylpicolinamide |
| 28 | 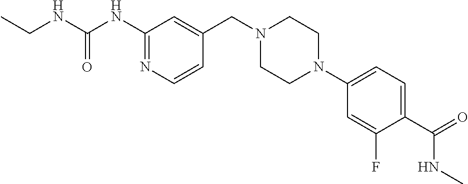 [SMILES: CCNC(=O)Nc1cc(CN2CCN(c3ccc(C(=O)NC)c(F)c3)CC2)ccn1] | 4-(4-((2-(3-ethylureido)pyridin-4- yl)methyl)piperazin-1-yl)-2-fluoro-N- methylbenzamide |
| 29 | 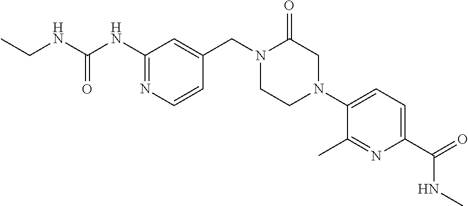 [SMILES: CCNC(=O)Nc1cc(CN2CCN(c3ccc(C(=O)NC)nc3C)CC2=O)ccn1] | 5-(4-((2-(3-ethylureido)pyridin-4- yl)methyl)-3-oxopiperazin-1-yl)-N,6- dimethylpicolinamide |
| 30 | 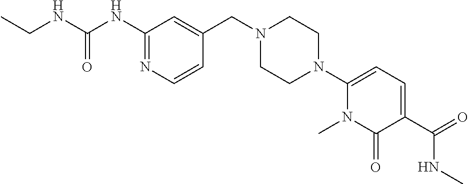 [SMILES: CCNC(=O)Nc1cc(CN2CCN(c3ccc(C(=O)NC)c(=O)n3C)CC2)ccn1] | 6-(4-((2-(3-ethylureido)pyridin-4- yl)methyl)piperazin-1-yl)-N,1-dimethyl- 2-oxo-1,2-dihydropyridine-3- carboxamide |
| 31 | 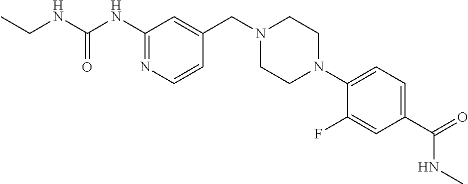 [SMILES: CCNC(=O)Nc1cc(CN2CCN(c3ccc(C(=O)NC)cc3F)CC2)ccn1] | 4-(4-((2-(3-ethylureido)pyridin-4- yl)methyl)piperazin-1-yl)-3-fluoro-N- methylbenzamide |
| 32 | 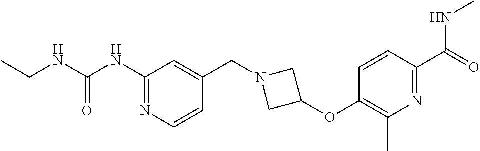 [SMILES: CCNC(=O)Nc1cc(CN2CC(Oc3ccc(C(=O)NC)nc3C)C2)ccn1] | 5-((1-((2-(3-ethylureido)pyridin-4- yl)methyl)azetidin-3-yl)oxy)-N,6- dimethylpicolinamide |
| 33 | 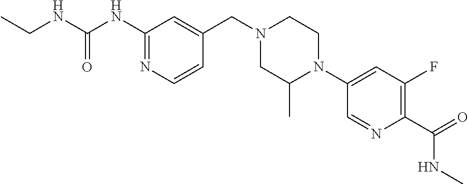 [SMILES: CCNC(=O)Nc1cc(CN2CCN(c3cnc(C(=O)NC)c(F)c3)C(C)C2)ccn1] | 5-(4-((2-(3-ethylureido)pyridin-4- yl)methyl)-2-methylpiperazin-1-yl)-3- fluoro-N-methylpicolinamide |
| 34 | 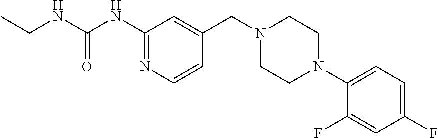 [SMILES: CCNC(=O)Nc1cc(CN2CCN(c3ccc(F)cc3F)CC2)ccn1] | 1-(4-((4-(2,4-difluorophenyl)piperazin-1- yl)methyl)pyridin-2-yl)-3-ethylurea |
| 35 | 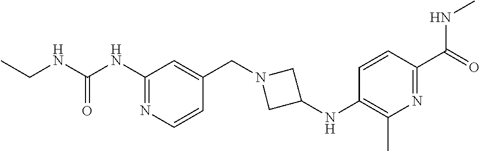 [SMILES: CCNC(=O)Nc1cc(CN2CC(Nc3ccc(C(=O)NC)nc3C)C2)ccn1] | 5-((1-((2-(3-ethylureido)pyridin-4- yl)methyl)azetidin-3-yl)amino)-N,6- dimethylpicolinamide |
| 36 | 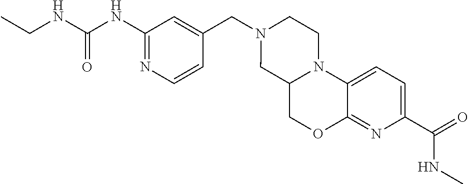 [SMILES: CCNC(=O)Nc1cc(CN2CCN3c4ccc(C(=O)NC)nc4OCC3C2)ccn1] | 3-((2-(3-ethylureido)pyridin-4- yl)methyl)-N-methyl-1,2,3,4,4a,5- hexahydropyrazino[1,2-d]pyrido[2,3- b][1,4]oxazine-8-carboxamide |
| 37 | 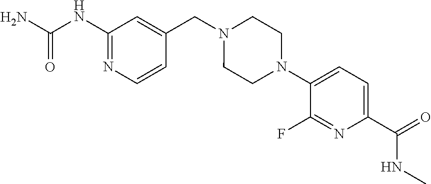 [SMILES: CNC(=O)c1ccc(N2CCN(Cc3ccnc(NC(N)=O)c3)CC2)c(F)n1] | 6-fluoro-N-methyl-5-(4-((2- ureidopyridin-4-yl)methyl)piperazin-1- yl)picolinamide |
| 38 | 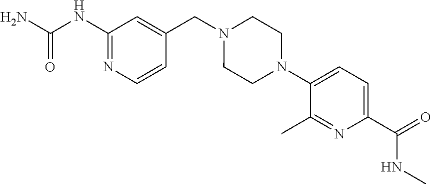 [SMILES: CNC(=O)c1ccc(N2CCN(Cc3ccnc(NC(N)=O)c3)CC2)c(C)n1] | N,6-dimethyl-5-(4-((2-ureidopyridin-4- yl)methyl)piperazin-1-yl)picolinamide |
| 39 | 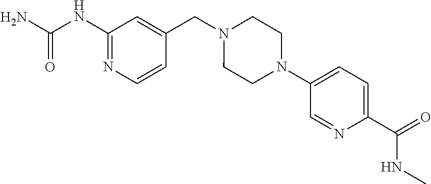 [SMILES: CNC(=O)c1ccc(N2CCN(Cc3ccnc(NC(N)=O)c3)CC2)cn1] | N-methyl-5-(4-((2-ureidopyridin-4- yl)methyl)piperazin-1-yl)picolinamide |
| 40 | 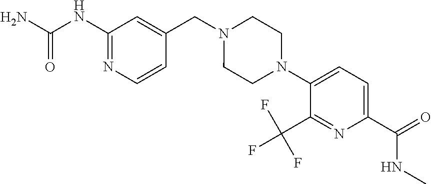 [SMILES: CNC(=O)c1ccc(N2CCN(Cc3ccnc(NC(N)=O)c3)CC2)c(C(F)(F)F)n1] | N-methyl-6-(trifluoromethyl)-5-(4-((2- ureidopyridin-4-yl)methyl)piperazin-1- yl)picolinamide |
| 41 | 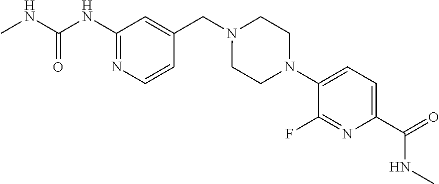 | 6-fluoro-N-methyl-5-(4-((2-(3- methylureido)pyridin-4- yl)methyl)piperazin-1-yl)picolinamide |
| 42 | 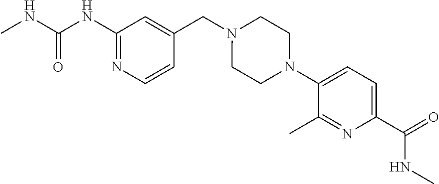 | N,6-dimethyl-5-(4-((2-(3- methylureido)pyridin-4- yl)methyl)piperazin-1-yl)picolinamide |
| 43 | 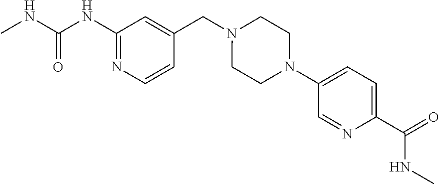 | N-methyl-5-(4-((2-(3- methylureido)pyridin-4- yl)methyl)piperazin-1-yl)picolinamide |
| 44 | 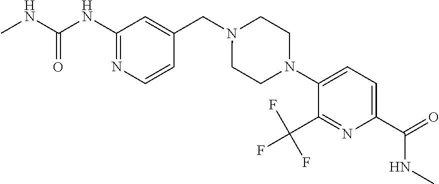 | N-methyl-5-(4-((2-(3- methylureido)pyridin-4- yl)methyl)piperazin-1-yl)-6- (trifluoromethyl)picolinamide |
| 45 | 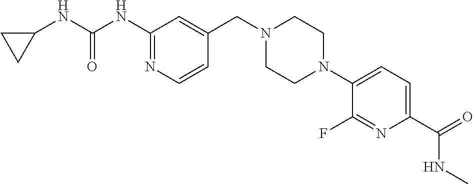 [SMILES: CNC(=O)c1ccc(N2CCN(Cc3ccnc(NC(=O)NC4CC4)c3)CC2)c(F)n1] | 5-(4-((2-(3-cyclopropylureido)pyridin-4- yl)methyl)piperazin-1-yl)-6-fluoro-N- methylpicolinamide |
| 46 | 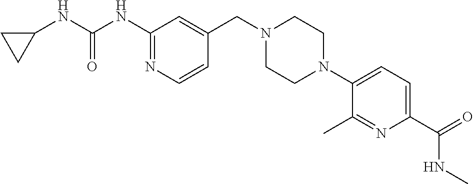 [SMILES: CNC(=O)c1ccc(N2CCN(Cc3ccnc(NC(=O)NC4CC4)c3)CC2)c(C)n1] | 5-(4-((2-(3-cyclopropylureido)pyridin-4- yl)methyl)piperazin-1-yl)-N,6- dimethylpicolinamide |
| 47 | 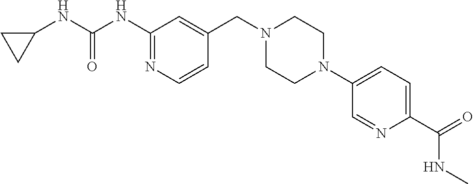 [SMILES: CNC(=O)c1ccc(N2CCN(Cc3ccnc(NC(=O)NC4CC4)c3)CC2)cn1] | 5-(4-((2-(3-cyclopropylureido)pyridin-4- yl)methyl)piperazin-1-yl)-N- methylpicolinamide |
| 48 | 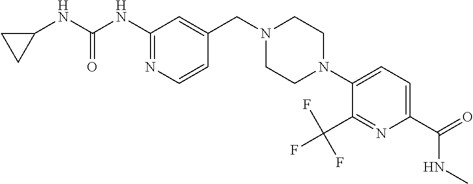 [SMILES: CNC(=O)c1ccc(N2CCN(Cc3ccnc(NC(=O)NC4CC4)c3)CC2)c(C(F)(F)F)n1] | 5-(4-((2-(3-cyclopropylureido)pyridin-4- yl)methyl)piperazin-1-yl)-N-methyl-6- (trifluoromethyl)picolinamide |
| 49 | 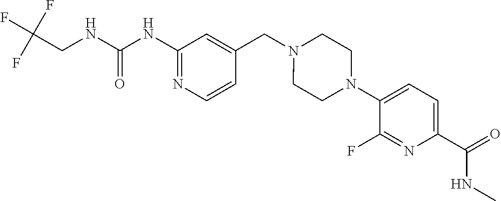 [SMILES: CNC(=O)c1ccc(N2CCN(Cc3ccnc(NC(=O)NCC(F)(F)F)c3)CC2)c(F)n1] | 6-fluoro-N-methyl-5-(4-((2-(3-(2,2,2- trifluoroethyl)ureido)pyridin-4- yl)methyl)piperazin-1-yl)picolinamide |
| 50 | 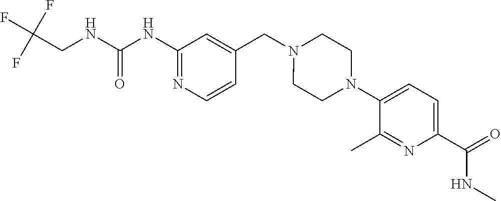 [SMILES: CNC(=O)c1ccc(N2CCN(Cc3ccnc(NC(=O)NCC(F)(F)F)c3)CC2)c(C)n1] | N,6-dimethyl-5-(4-((2-(3-(2,2,2- trifluoroethyl)ureido)pyridin-4- yl)methyl)piperazin-1-yl)picolinamide |
| 51 | 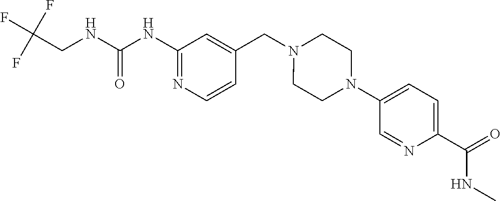 [SMILES: CNC(=O)c1ccc(N2CCN(Cc3ccnc(NC(=O)NCC(F)(F)F)c3)CC2)cn1] | N-methyl-5-(4-((2-(3-(2,2,2- trifluoroethyl)ureido)pyridin-4- yl)methyl)piperazin-1-yl)picolinamide |
| 52 | 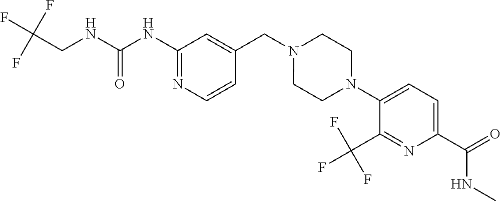 [SMILES: CNC(=O)c1ccc(N2CCN(Cc3ccnc(NC(=O)NCC(F)(F)F)c3)CC2)c(C(F)(F)F)n1] | N-methyl-5-(4-((2-(3-(2,2,2- trifluoroethyl)ureido)pyridin-4- yl)methyl)piperazin-1-yl)-6- (trifluoromethyl)picolinamide |
| 53 | 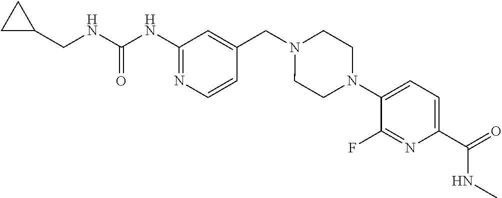 [SMILES: CNC(=O)c1ccc(N2CCN(Cc3ccnc(NC(=O)NCC4CC4)c3)CC2)c(F)n1] | 5-(4-((2-(3- (cyclopropylmethyl)ureido)pyridin-4- yl)methyl)piperazin-1-yl)-6-fluoro-N- methylpicolinamide |
| 54 | 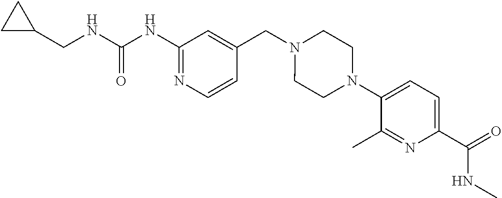 [SMILES: CNC(=O)c1ccc(N2CCN(Cc3ccnc(NC(=O)NCC4CC4)c3)CC2)c(C)n1] | 5-(4-((2-(3- (cyclopropylmethyl)ureido)pyridin-4- yl)methyl)piperazin-1-yl)-N,6- dimethylpicolinamide |
| 55 | 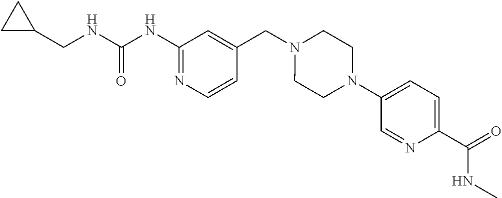 [SMILES: CNC(=O)c1ccc(N2CCN(Cc3ccnc(NC(=O)NCC4CC4)c3)CC2)cn1] | 5-(4-((2-(3- (cyclopropylmethyl)ureido)pyridin-4- yl)methyl)piperazin-1-yl)-N- methylpicolinamide |
| 56 | 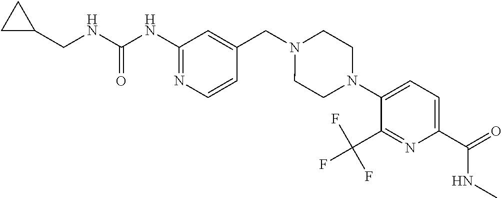 [SMILES: CNC(=O)c1ccc(N2CCN(Cc3ccnc(NC(=O)NCC4CC4)c3)CC2)c(C(F)(F)F)n1] | 5-(4-((2-(3- (cyclopropylmethyl)ureido)pyridin-4- yl)methyl)piperazin-1-yl)-N-methyl-6- (trifluoromethyl)picolinamide |
| 57 | 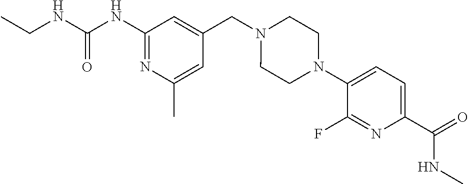 [SMILES: CCNC(=O)Nc1cc(CN2CCN(c3ccc(C(=O)NC)nc3F)CC2)cc(C)n1] | 5-(4-((2-(3-ethylureido)-6-methylpyridin- 4-yl)methyl)piperazin-1-yl)-6-fluoro-N- methylpicolinamide |
| 58 | 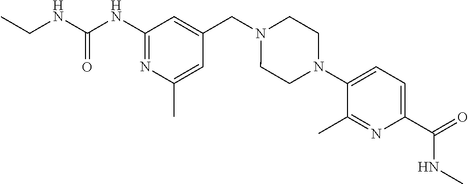 [SMILES: CCNC(=O)Nc1cc(CN2CCN(c3ccc(C(=O)NC)nc3C)CC2)cc(C)n1] | 5-(4-((2-(3-ethylureido)-6-methylpyridin- 4-yl)methyl)piperazin-1-yl)-N,6- dimethylpicolinamide |
| 59 | 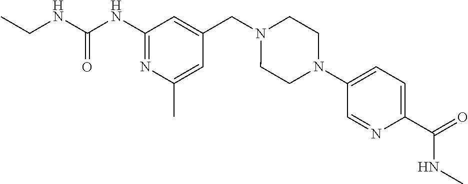 [SMILES: CCNC(=O)Nc1cc(CN2CCN(c3ccc(C(=O)NC)nc3)CC2)cc(C)n1] | 5-(4-((2-(3-ethylureido)-6-methylpyridin- 4-yl)methyl)piperazin-1-yl)-N- methylpicolinamide |
| 60 | 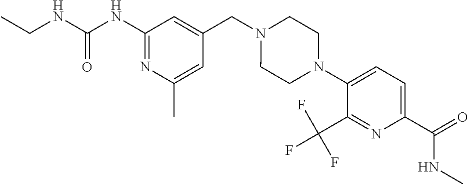 [SMILES: CCNC(=O)Nc1cc(CN2CCN(c3ccc(C(=O)NC)nc3C(F)(F)F)CC2)cc(C)n1] | 5-(4-((2-(3-ethylureido)-6-methylpyridin- 4-yl)methyl)piperazin-1-yl)-N-methyl-6- (trifluoromethyl)picolinamide |
| 61 | 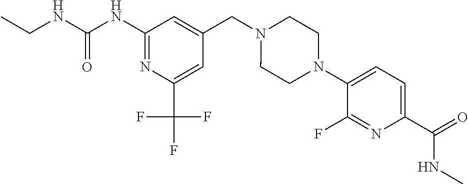 [SMILES: CCNC(=O)Nc1cc(CN2CCN(c3ccc(C(=O)NC)nc3F)CC2)cc(C(F)(F)F)n1] | 5-(4-((2-(3-ethylureido)-6- (trifluoromethyl)pyridin-4- yl)methyl)piperazin-1-yl)-6-fluoro-N- methylpicolinamide |
| 62 | 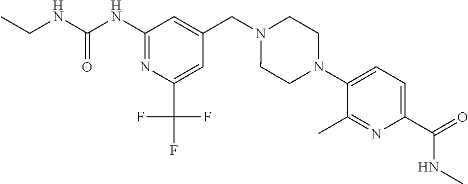 [SMILES: CCNC(=O)Nc1cc(CN2CCN(c3ccc(C(=O)NC)nc3C)CC2)cc(C(F)(F)F)n1] | 5-(4-((2-(3-ethylureido)-6- (trifluoromethyl)pyridin-4- yl)methyl)piperazin-1-yl)-N,6- dimethylpicolinamide |
| 63 | 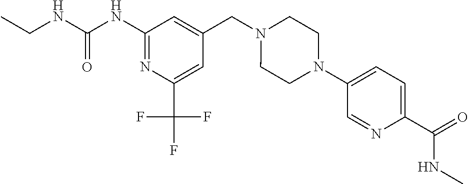 [SMILES: CCNC(=O)Nc1cc(CN2CCN(c3ccc(C(=O)NC)nc3)CC2)cc(C(F)(F)F)n1] | 5-(4-((2-(3-ethylureido)-6- (trifluoromethyl)pyridin-4- yl)methyl)piperazin-1-yl)-N- methylpicolinamide |
| 64 | 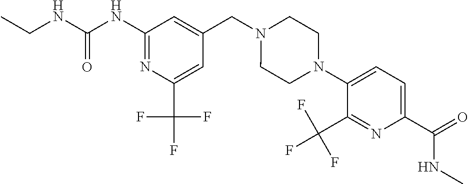 [SMILES: CCNC(=O)Nc1cc(CN2CCN(c3ccc(C(=O)NC)nc3C(F)(F)F)CC2)cc(C(F)(F)F)n1] | 5-(4-((2-(3-ethylureido)-6- (trifluoromethyl)pyridin-4- yl)methyl)piperazin-1-yl)-N-methyl-6- (trifluoromethyl)picolinamide |
| 65 | 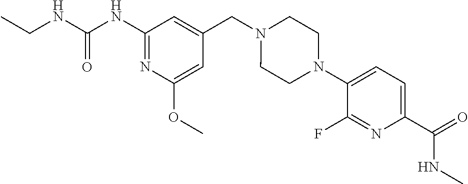 [SMILES: CCNC(=O)Nc1cc(CN2CCN(c3ccc(C(=O)NC)nc3F)CC2)cc(OC)n1] | 5-(4-((2-(3-ethylureido)-6- methoxypyridin-4-yl)methyl)piperazin-1- yl)-6-fluoro-N-methylpicolinamide |
| 66 | 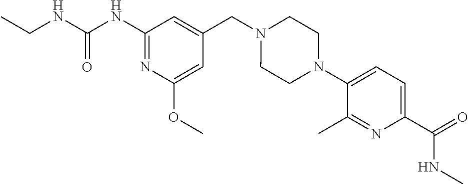 [SMILES: CCNC(=O)Nc1cc(CN2CCN(c3ccc(C(=O)NC)nc3C)CC2)cc(OC)n1] | 5-(4-((2-(3-ethylureido)-6- methoxypyridin-4-yl)methyl)piperazin-1- yl)-N,6-dimethylpicolinamide |
| 67 | 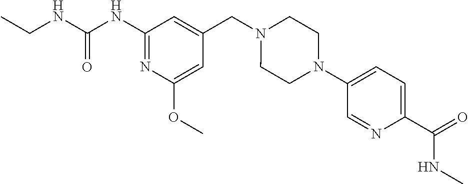 [SMILES: CCNC(=O)Nc1cc(CN2CCN(c3ccc(C(=O)NC)nc3)CC2)cc(OC)n1] | 5-(4-((2-(3-ethylureido)-6- methoxypyridin-4-yl)methyl)piperazin-1- yl)-N-methylpicolinamide |
| 68 | 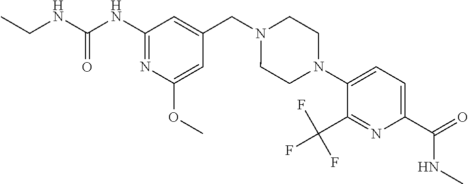 [SMILES: CCNC(=O)Nc1cc(CN2CCN(c3ccc(C(=O)NC)nc3C(F)(F)F)CC2)cc(OC)n1] | 5-(4-((2-(3-ethylureido)-6- methoxypyridin-4-yl)methyl)piperazin-1- yl)-N-methyl-6- (trifluoromethyl)picolinamide |
| 69 | 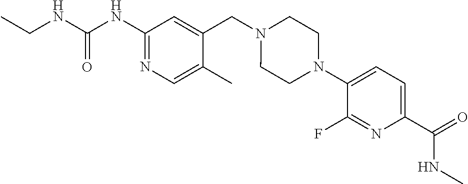 [SMILES: CCNC(=O)Nc1cc(CN2CCN(c3ccc(C(=O)NC)nc3F)CC2)c(C)cn1] | 5-(4-((2-(3-ethylureido)-5-methylpyridin- 4-yl)methyl)piperazin-1-yl)-6-fluoro-N- methylpicolinamide |
| 70 | 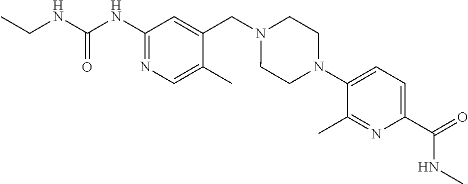 [SMILES: CCNC(=O)Nc1cc(CN2CCN(c3ccc(C(=O)NC)nc3C)CC2)c(C)cn1] | 5-(4-((2-(3-ethylureido)-5-methylpyridin- 4-yl)methyl)piperazin-1-yl)-N,6- dimethylpicolinamide |
| 71 | 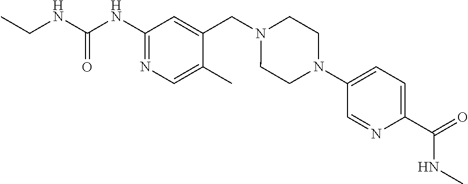 [SMILES: CCNC(=O)Nc1cc(CN2CCN(c3ccc(C(=O)NC)nc3)CC2)c(C)cn1] | 5-(4-((2-(3-ethylureido)-5-methylpyridin- 4-yl)methyl)piperazin-1-yl)-N- methylpicolinamide |
| 72 | 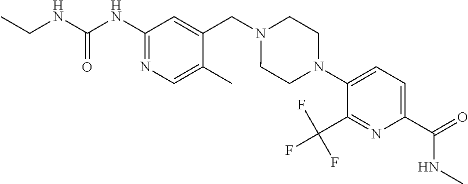 [SMILES: CCNC(=O)Nc1cc(CN2CCN(c3ccc(C(=O)NC)nc3C(F)(F)F)CC2)c(C)cn1] | 5-(4-((2-(3-ethylureido)-5-methylpyridin- 4-yl)methyl)piperazin-1-yl)-N-methyl-6- (trifluoromethyl)picolinamide |
| 73 | 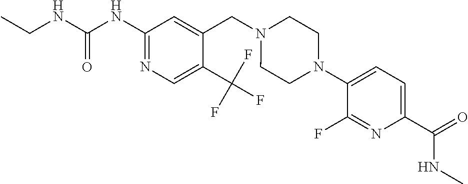 [SMILES: CCNC(=O)Nc1cc(CN2CCN(c3ccc(C(=O)NC)nc3F)CC2)c(C(F)(F)F)cn1] | 5-(4-((2-(3-ethylureido)-5- (trifluoromethyl)pyridin-4- yl)methyl)piperazin-1-yl)-6-fluoro-N- methylpicolinamide |
| 74 | 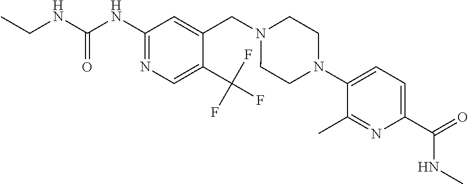 [SMILES: CCNC(=O)Nc1cc(CN2CCN(c3ccc(C(=O)NC)nc3C)CC2)c(C(F)(F)F)cn1] | 5-(4-((2-(3-ethylureido)-5- (trifluoromethyl)pyridin-4- yl)methyl)piperazin-1-yl)-N,6- dimethylpicolinamide |
| 75 | 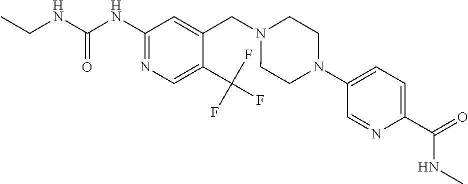 [SMILES: CCNC(=O)Nc1cc(CN2CCN(c3ccc(C(=O)NC)nc3)CC2)c(C(F)(F)F)cn1] | 5-(4-((2-(3-ethylureido)-5- (trifluoromethyl)pyridin-4- yl)methyl)piperazin-1-yl)-N- methylpicolinamide |
| 76 | 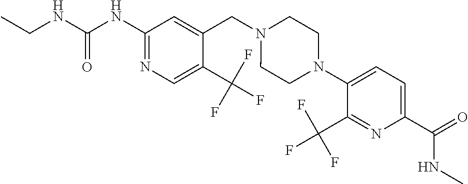 [SMILES: CCNC(=O)Nc1cc(CN2CCN(c3ccc(C(=O)NC)nc3C(F)(F)F)CC2)c(C(F)(F)F)cn1] | 5-(4-((2-(3-ethylureido)-5- (trifluoromethyl)pyridin-4- yl)methyl)piperazin-1-yl)-N-methyl-6- (trifluoromethyl)picolinamide |
| 77 | 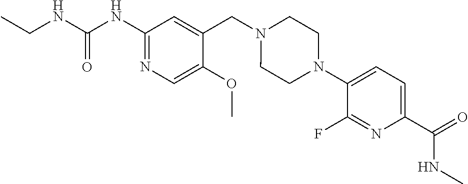 [SMILES: CCNC(=O)Nc1cc(CN2CCN(c3ccc(C(=O)NC)nc3F)CC2)c(OC)cn1] | 5-(4-((2-(3-ethylureido)-5- methoxypyridin-4-yl)methyl)piperazin-1- yl)-6-fluoro-N-methylpicolinamide |
| 78 | 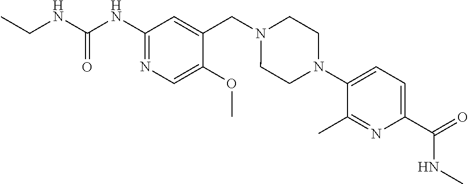 [SMILES: CCNC(=O)Nc1cc(CN2CCN(c3ccc(C(=O)NC)nc3C)CC2)c(OC)cn1] | 5-(4-((2-(3-ethylureido)-5- methoxypyridin-4-yl)methyl)piperazin-1- yl)-N,6-dimethylpicolinamide |
| 79 | 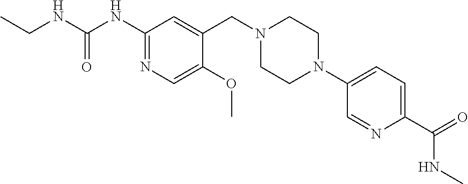 [SMILES: CCNC(=O)Nc1cc(CN2CCN(c3ccc(C(=O)NC)nc3)CC2)c(OC)cn1] | 5-(4-((2-(3-ethylureido)-5- methoxypyridin-4-yl)methyl)piperazin-1- yl)-N-methylpicolinamide |
| 80 | 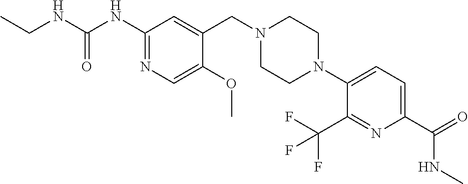 [SMILES: CCNC(=O)Nc1cc(CN2CCN(c3ccc(C(=O)NC)nc3C(F)(F)F)CC2)c(OC)cn1] | 5-(4-((2-(3-ethylureido)-5- methoxypyridin-4-yl)methyl)piperazin-1- yl)-N-methyl-6- (trifluoromethyl)picolinamide |
| 81 | 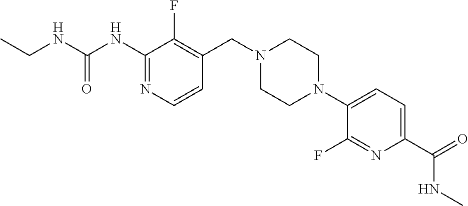 [SMILES: CCNC(=O)Nc1nccc(CN2CCN(c3ccc(C(=O)NC)nc3F)CC2)c1F] | 5-(4-((2-(3-ethylureido)-3-fluoropyridin- 4-yl)methyl)piperazin-1-yl)-6-fluoro-N- methylpicolinamide |
| 82 | 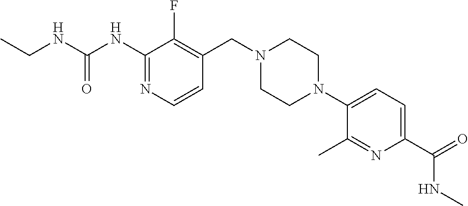 [SMILES: CCNC(=O)Nc1nccc(CN2CCN(c3ccc(C(=O)NC)nc3C)CC2)c1F] | 5-(4-((2-(3-ethylureido)-3-fluoropyridin- 4-yl)methyl)piperazin-1-yl)-N,6- dimethylpicolinamide |
| 83 | 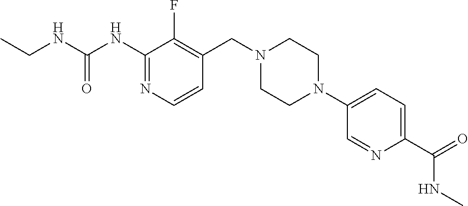 [SMILES: CCNC(=O)Nc1nccc(CN2CCN(c3ccc(C(=O)NC)nc3)CC2)c1F] | 5-(4-((2-(3-ethylureido)-3-fluoropyridin- 4-yl)methyl)piperazin-1-yl)-N- methylpicolinamide |
| 84 | 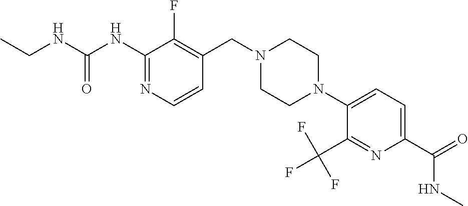 [SMILES: CCNC(=O)Nc1nccc(CN2CCN(c3ccc(C(=O)NC)nc3C(F)(F)F)CC2)c1F] | 5-(4-((2-(3-ethylureido)-3-fluoropyridin- 4-yl)methyl)piperazin-1-yl)-N-methyl-6- (trifluoromethyl)picolinamide |
| 85 | 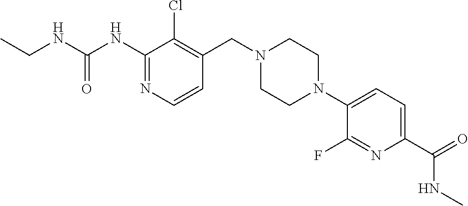 [SMILES: CCNC(=O)Nc1nccc(CN2CCN(c3ccc(C(=O)NC)nc3F)CC2)c1Cl] | 5-(4-((3-chloro-2-(3-ethylureido)pyridin- 4-yl)methyl)piperazin-1-yl)-6-fluoro-N- methylpicolinamide |
| 86 | 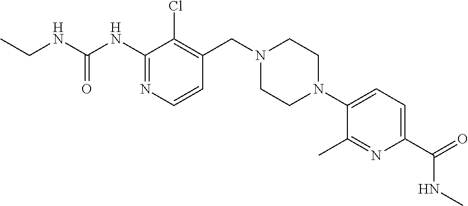 [SMILES: CCNC(=O)Nc1nccc(CN2CCN(c3ccc(C(=O)NC)nc3C)CC2)c1Cl] | 5-(4-((3-chloro-2-(3-ethylureido)pyridin- 4-yl)methyl)piperazin-1-yl)-N,6- dimethylpicolinamide |
| 87 | 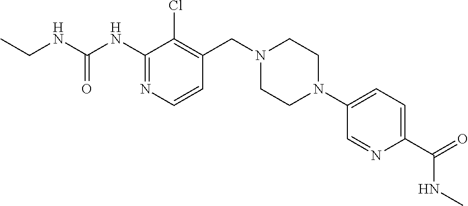 [SMILES: CCNC(=O)Nc1nccc(CN2CCN(c3ccc(C(=O)NC)nc3)CC2)c1Cl] | 5-(4-((3-chloro-2-(3-ethylureido)pyridin- 4-yl)methyl)piperazin-1-yl)-N- methylpicolinamide |
| 88 | 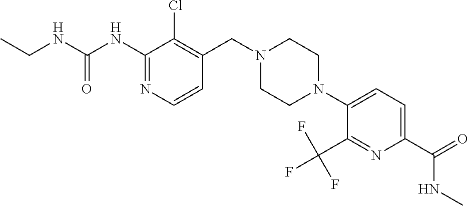 [SMILES: CCNC(=O)Nc1nccc(CN2CCN(c3ccc(C(=O)NC)nc3C(F)(F)F)CC2)c1Cl] | 5-(4-((3-chloro-2-(3-ethylureido)pyridin- 4-yl)methyl)piperazin-1-yl)-N-methyl-6- (trifluoromethyl)picolinamide |
| 89 | 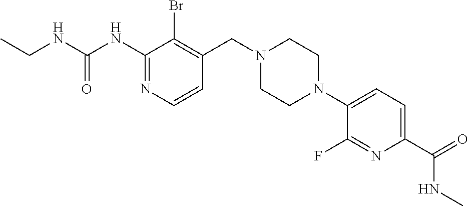 [SMILES: CCNC(=O)Nc1nccc(CN2CCN(c3ccc(C(=O)NC)nc3F)CC2)c1Br] | 5-(4-((3-bromo-2-(3-ethylureido)pyridin- 4-yl)methyl)piperazin-1-yl)-6-fluoro-N- methylpicolinamide |
| 90 | 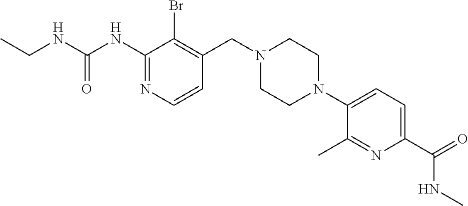 [SMILES: CCNC(=O)Nc1nccc(CN2CCN(c3ccc(C(=O)NC)nc3C)CC2)c1Br] | 5-(4-((3-bromo-2-(3-ethylureido)pyridin- 4-yl)methyl)piperazin-1-yl)-N,6- dimethylpicolinamide |
| 91 | 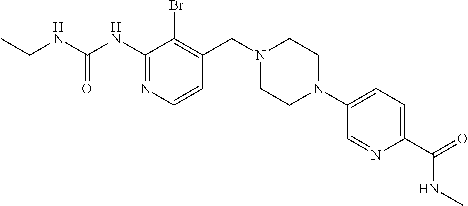 [SMILES: CCNC(=O)Nc1nccc(CN2CCN(c3ccc(C(=O)NC)nc3)CC2)c1Br] | 5-(4-((3-bromo-2-(3-ethylureido)pyridin- 4-yl)methyl)piperazin-1-yl)-N- methylpicolinamide |
| 92 | 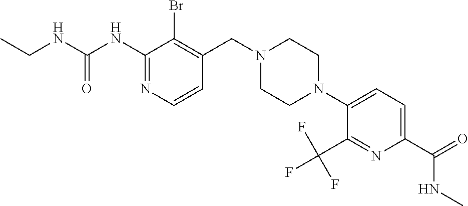 [SMILES: CCNC(=O)Nc1nccc(CN2CCN(c3ccc(C(=O)NC)nc3C(F)(F)F)CC2)c1Br] | 5-(4-((3-bromo-2-(3-ethylureido)pyridin- 4-yl)methyl)piperazin-1-yl)-N-methyl-6- (trifluoromethyl)picolinamide |
| 93 | 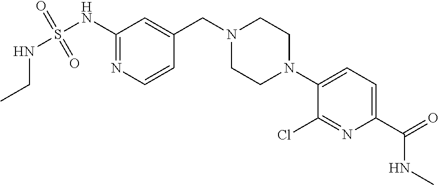 [SMILES: CCNS(=O)(=O)Nc1cc(CN2CCN(c3ccc(C(=O)NC)nc3Cl)CC2)ccn1] | 6-chloro-5-(4-((2-((N- ethylsulfamoyl)amino)pyridin-4- yl)methyl)piperazin-1-yl)-N- methylpicolinamide |
| 94 | 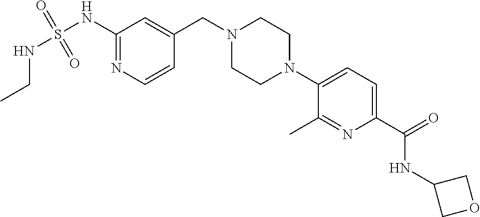 [SMILES: CCNS(=O)(=O)Nc1cc(CN2CCN(c3ccc(C(=O)NC4COC4)nc3C)CC2)ccn1] | 5-(4-((2-((N- ethylsulfamoyl)amino)pyridin-4- yl)methyl)piperazin-1-yl)-6-methyl-N- (oxetan-3-yl)picolinamide |
| 95 | 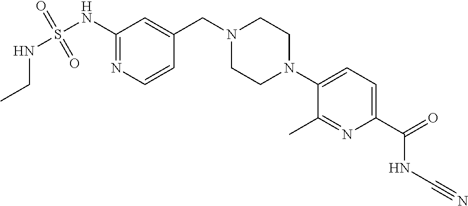 [SMILES: CCNS(=O)(=O)Nc1cc(CN2CCN(c3ccc(C(=O)NC#N)nc3C)CC2)ccn1] | N-cyano-5-(4-((2-((N- ethylsulfamoyl)amino)pyridin-4- yl)methyl)piperazin-1-yl)-6- methylpicolinamide |
| 96 | 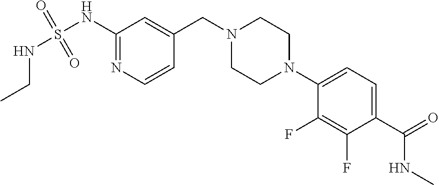 [SMILES: CCNS(=O)(=O)Nc1cc(CN2CCN(c3ccc(C(=O)NC)c(F)c3F)CC2)ccn1] | 4-(4-((2-((N- ethylsulfamoyl)amino)pyridin-4- yl)methyl)piperazin-1-yl)-2,3-difluoro-N- methylbenzamide |
| 97 | 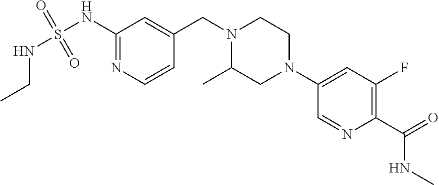 [SMILES: CCNS(=O)(=O)Nc1cc(CN2CCN(c3cnc(C(=O)NC)c(F)c3)CC2C)ccn1] | 5-(4-((2-((N- ethylsulfamoyl)amino)pyridin-4- yl)methyl)-3-methylpiperazin-1-yl)-3- fluoro-N-methylpicolinamide |
| 98 | 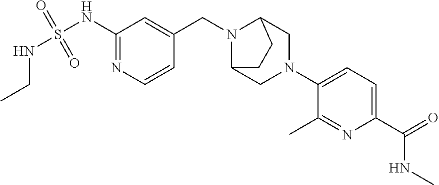 [SMILES: CCNS(=O)(=O)Nc1cc(CN2C3CCC2CN(c2ccc(C(=O)NC)nc2C)C3)ccn1] | 5-(8-((2-((N- ethylsulfamoyl)amino)pyridin-4- yl)methyl)-3,8-diazabicyclo[3.2.1]octan- 3-yl)-N,6-dimethylpicolinamide |
| 99 | 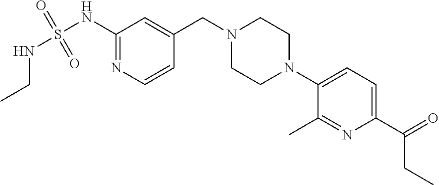 [SMILES: CCNS(=O)(=O)Nc1cc(CN2CCN(c3ccc(C(=O)CC)nc3C)CC2)ccn1] | 1-[5-(4-{[2-(ethylaminosulfonylamino)-4- pyridyl]methyl}-1-piperazinyl)-6-methyl- 2-pyridyl]-1-propanone |
| 100 | 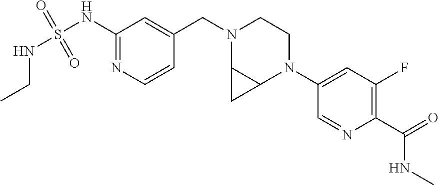 [SMILES: CCNS(=O)(=O)Nc1cc(CN2CCN(c3cnc(C(=O)NC)c(F)c3)C3CC32)ccn1] | 5-(5-((2-((N- ethylsulfamoyl)amino)pyridin-4- yl)methyl)-2,5-diazabicyclo[4.1.0]heptan- 2-yl)-3-fluoro-N-methylpicolinamide |
| 101 | 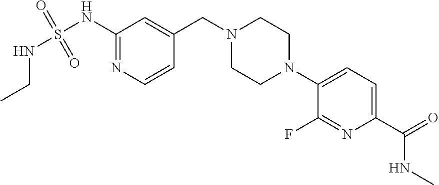 [SMILES: CCNS(=O)(=O)Nc1cc(CN2CCN(c3ccc(C(=O)NC)nc3F)CC2)ccn1] | 5-(4-((2-((N- ethylsulfamoyl)amino)pyridin-4- yl)methyl)piperazin-1-yl)-6-fluoro-N- methylpicolinamide |
| 102 | 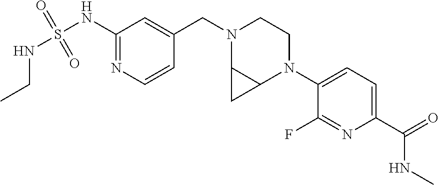 [SMILES: CCNS(=O)(=O)Nc1cc(CN2CCN(c3ccc(C(=O)NC)nc3F)C3CC32)ccn1] | 5-(5-((2-((N- ethylsulfamoyl)amino)pyridin-4- yl)methyl)-2,5-diazabicyclo[4.1.0]heptan- 2-yl)-6-fluoro-N-methylpicolinamide |
| 103 | 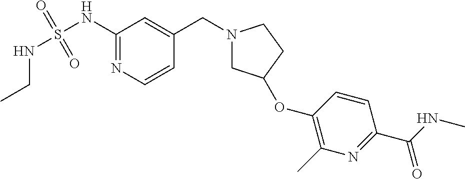 [SMILES: CCNS(=O)(=O)Nc1cc(CN2CCC(Oc3ccc(C(=O)NC)nc3C)C2)ccn1] | 5-((1-((2-((N- ethylsulfamoyl)amino)pyridin-4- yl)methyl)pyrrolidin-3-yl)oxy)-N,6- dimethylpicolinamide |
| 104 | 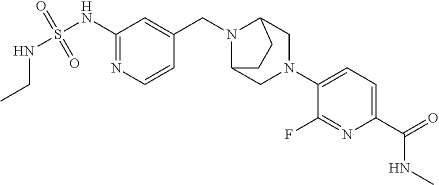 [SMILES: CCNS(=O)(=O)Nc1cc(CN2C3CCC2CN(c2ccc(C(=O)NC)nc2F)C3)ccn1] | 5-(8-((2-((N- ethylsulfamoyl)amino)pyridin-4- yl)methyl)-3,8-diazabicyclo[3.2.1]octan- 3-yl)-6-fluoro-N-methylpicolinamide |
| 105 | 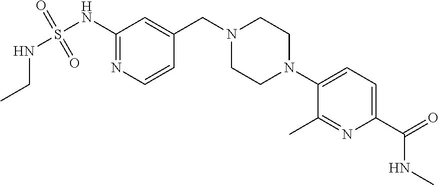 [SMILES: CCNS(=O)(=O)Nc1cc(CN2CCN(c3ccc(C(=O)NC)nc3C)CC2)ccn1] | 5-(4-((2-((N- ethylsulfamoyl)amino)pyridin-4- yl)methyl)piperazin-1-yl)-N,6- dimethylpicolinamide |
| 106 | 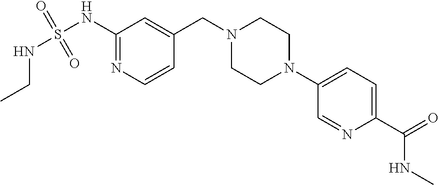 [SMILES: CCNS(=O)(=O)Nc1cc(CN2CCN(c3ccc(C(=O)NC)nc3)CC2)ccn1] | 5-(4-((2-((N- ethylsulfamoyl)amino)pyridin-4- yl)methyl)piperazin-1-yl)-N- methylpicolinamide |
| 107 | 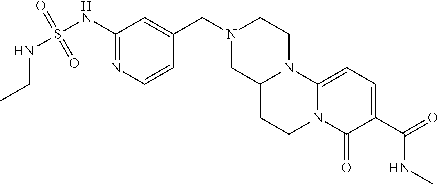 [SMILES: CCNS(=O)(=O)Nc1cc(CN2CCN3c4ccc(C(=O)NC)c(=O)n4CCC3C2)ccn1] | 3-((2-((N-ethylsulfamoyl)amino)pyridin- 4-yl)methyl)-N-methyl-8-oxo- 2,3,4,4a,5,6-hexahydro-1H,8H- pyrazino[1,2-c]pyrido[1,2-a]pyrimidine- 9-carboxamide |
| 108 | 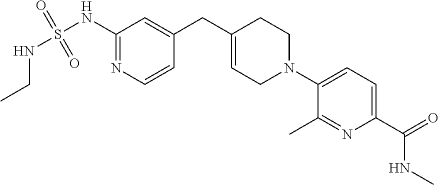 [SMILES: CCNS(=O)(=O)Nc1cc(CC2=CCN(c3ccc(C(=O)NC)nc3C)CC2)ccn1] | 4-((2-((N-etbylsulfamoyl)amino)pyridin- 4-yl)methyl)-N,2′-dimethyl-3,6-dihydro- 2H-[1,3′-bipyridine]-6′-carboxamide |
| 109 | 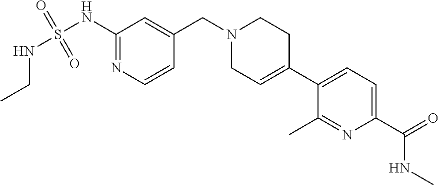 [SMILES: CCNS(=O)(=O)Nc1cc(CN2CC=C(c3ccc(C(=O)NC)nc3C)CC2)ccn1] | 1′-((2-((N-ethylsulfamoyl)amino)pyridin- 4-yl)methyl)-N,2-dimethyl-1′,2′,3′,6′- tetrahydro-[3,4′-bipyridine]-6- carboxamide |
| 110 | 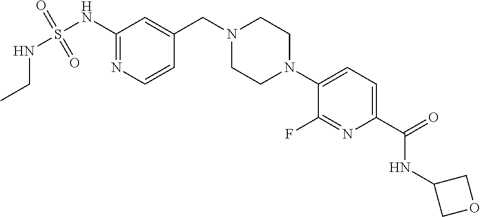 [SMILES: CCNS(=O)(=O)Nc1cc(CN2CCN(c3ccc(C(=O)NC4COC4)nc3F)CC2)ccn1] | 5-(4-((2-((N- ethylsulfamoyl)amino)pyridin-4- yl)methyl)piperazin-1-yl)-6-fluoro-N- (oxetan-3-yl)picolinamide |
| 111 | 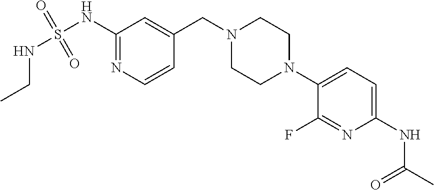 [SMILES: CCNS(=O)(=O)Nc1cc(CN2CCN(c3ccc(NC(C)=O)nc3F)CC2)ccn1] | N-(5-(4-((2-((N- ethylsulfamoyl)amino)pyridin-4- yl)methyl)piperazin-1-yl)-6- fluoropyridin-2-yl)acetamide |
| 112 | 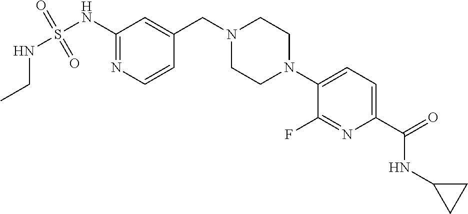 [SMILES: CCNS(=O)(=O)Nc1cc(CN2CCN(c3ccc(C(=O)NC4CC4)nc3F)CC2)ccn1] | N-cyclopropyl-5-(4-((2-((N- ethylsulfamoyl)amino)pyridin-4- yl)methyl)piperazin-1-yl)-6- fluoropicolinamide |
| 113 | 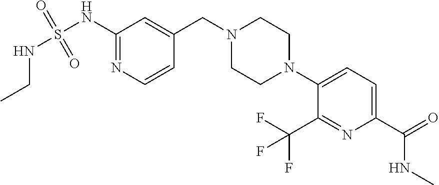 [SMILES: CCNS(=O)(=O)Nc1cc(CN2CCN(c3ccc(C(=O)NC)nc3C(F)(F)F)CC2)ccn1] | 5-(4-((2-((N- ethylsulfamoyl)amino)pyridin-4- yl)methyl)piperazin-1-yl)-N-methyl-6- (trifluoromethyl)picolinamide |
| 114 | 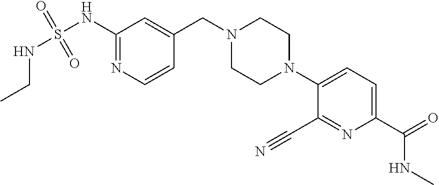 [SMILES: CCNS(=O)(=O)Nc1cc(CN2CCN(c3ccc(C(=O)NC)nc3C#N)CC2)ccn1] | 6-cyano-5-(4-((2-((N- ethylsulfamoyl)amino)pyridin-4- yl)methyl)piperazin-1-yl)-N- methylpicolinamide |
| 115 | 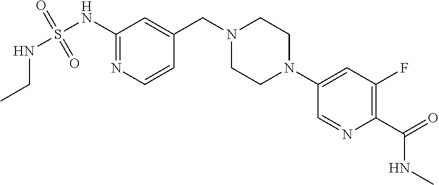 [SMILES: CCNS(=O)(=O)Nc1cc(CN2CCN(c3cnc(C(=O)NC)c(F)c3)CC2)ccn1] | 5-(4-((2-((N- ethylsulfamoyl)amino)pyridin-4- yl)methyl)piperazin-1-yl)-3-fluoro-N- methylpicolinamide |
| 116 | 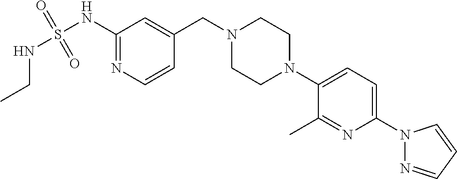 [SMILES: CCNS(=O)(=O)Nc1cc(CN2CCN(c3ccc(-n4cccn4)nc3C)CC2)ccn1] | 2-(ethylaminosulfonylamino)-4-({4-[2- methyl-6-(1-pyrazolyl)-3-pyridyl]-1- piperazinyl} methyl)pyridine |
| 117 | 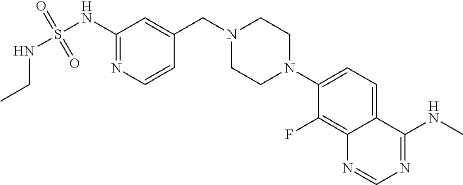 [SMILES: CCNS(=O)(=O)Nc1cc(CN2CCN(c3ccc4c(NC)ncnc4c3F)CC2)ccn1] | 7-(4-{[2-(ethylaminosulfonylamino)-4- pyridyl]methyl}-1-piperazinyl)-8-fluoro- 4-(methylamino)quinazoline |
| 118 | 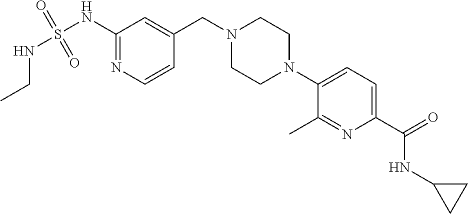 [SMILES: CCNS(=O)(=O)Nc1cc(CN2CCN(c3ccc(C(=O)NC4CC4)nc3C)CC2)ccn1] | N-cyclopropyl-5-(4-((2-((N- ethylsulfamoyl)amino)pyridin-4- yl)methyl)piperazin-1-yl)-6- methylpicolinamide |
| 119 | 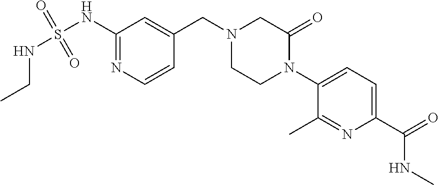 [SMILES: CCNS(=O)(=O)Nc1cc(CN2CCN(c3ccc(C(=O)NC)nc3C)C(=O)C2)ccn1] | 5-(4-((2-((N- ethylsulfamoyl)amino)pyridin-4- yl)methyl)-2-oxopiperazin-1-yl)-N,6- dimethylpicolinamide |
| 120 | 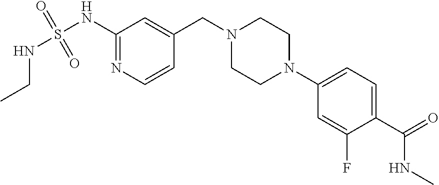 [SMILES: CCNS(=O)(=O)Nc1cc(CN2CCN(c3ccc(C(=O)NC)c(F)c3)CC2)ccn1] | 4-(4-((2-((N- ethylsulfamoyl)amino)pyridin-4- yl)methyl)piperazin-1-yl)-2-fluoro-N- methylbenzamide |
| 121 | 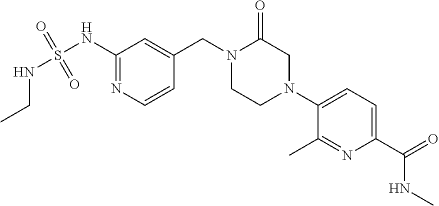 [SMILES: CCNS(=O)(=O)Nc1cc(CN2CCN(c3ccc(C(=O)NC)nc3C)CC2=O)ccn1] | 5-(4-((2-((N- ethylsulfamoyl)amino)pyridin-4- yl)methyl)-3-oxopiperazin-1-yl)-N,6- dimethylpicolinamide |
| 122 | 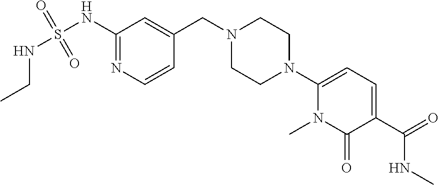 [SMILES: CCNS(=O)(=O)Nc1cc(CN2CCN(c3ccc(C(=O)NC)c(=O)n3C)CC2)ccn1] | 6-(4-((2-((N- ethylsulfamoyl)amino)pyridin-4- yl)methyl)piperazin-1-yl)-N,1-dimethyl- 2-oxo-1,2-dihydropyridine-3- carboxamide |
| 123 | 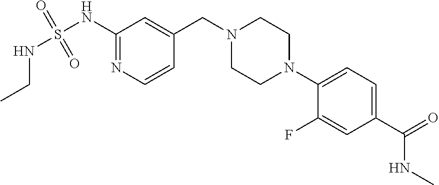 [SMILES: CCNS(=O)(=O)Nc1cc(CN2CCN(c3ccc(C(=O)NC)cc3F)CC2)ccn1] | 4-(4-((2-((N- ethylsulfamoyl)amino)pyridin-4- yl)methyl)piperazin-1-yl)-3-fluoro-N- methylbenzamide |
| 124 | 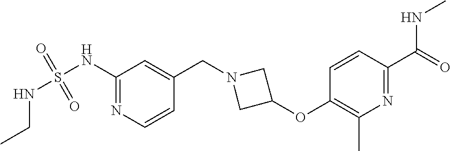 [SMILES: CCNS(=O)(=O)Nc1cc(CN2CC(Oc3ccc(C(=O)NC)nc3C)C2)ccn1] | 5-((1-((2-((N- ethylsulfamoyl)amino)pyridin-4- yl)methyl)azetidin-3-yl)oxy)-N,6- dimethylpicolinamide |
| 125 | 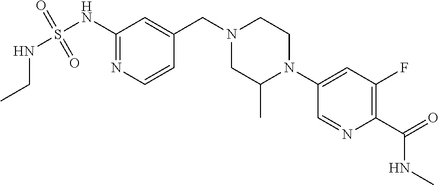 [SMILES: CCNS(=O)(=O)Nc1cc(CN2CCN(c3cnc(C(=O)NC)c(F)c3)C(C)C2)ccn1] | 5-(4-((2-((N- ethylsulfamoyl)amino)pyridin-4- yl)methyl)-2-methylpiperazin-1-yl)-3- fluoro-N-methylpicolinamide |
| 126 | 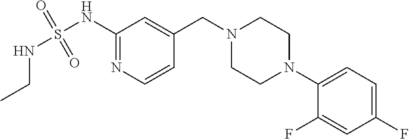 [SMILES: CCNS(=O)(=O)Nc1cc(CN2CCN(c3ccc(F)cc3F)CC2)ccn1] | 4-{[4-(2,4-difluorophenyl)-1- piperazinyl]methyl}-2- (ethylaminosulfonylamino)pyridine |
| 127 | 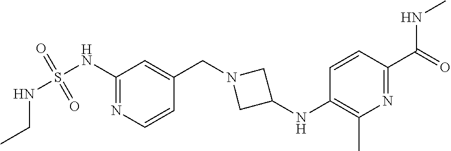 [SMILES: CCNS(=O)(=O)Nc1cc(CN2CC(Nc3ccc(C(=O)NC)nc3C)C2)ccn1] | 5-((1-((2-((N- ethylsulfamoyl)amino)pyridin-4- yl)methyl)azetidin-3-yl)amino)-N,6- dimethylpicolinamide |
| 128 | 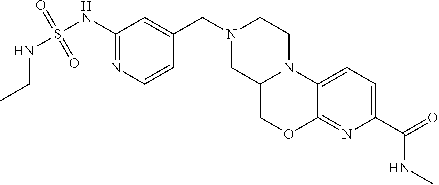 [SMILES: CCNS(=O)(=O)Nc1cc(CN2CCN3c4ccc(C(=O)NC)nc4OCC3C2)ccn1] | 3-((2-((N-ethylsulfamoyl)amino)pyridin- 4-yl)methyl)-N-methyl-1,2,3,4,4a,5- hexabydropyrazino[1,2-d]pyrido[2,3- b][1,4]oxazine-8-carboxamide |
| 129 | 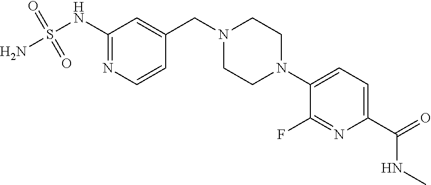 [SMILES: CNC(=O)c1ccc(N2CCN(Cc3ccnc(NS(N)(=O)=O)c3)CC2)c(F)n1] | 6-fluoro-N-methyl-5-(4-((2- (sulfamoylamino)pyridin-4- yl)methyl)piperazin-1-yl)picolinamide |
| 130 | 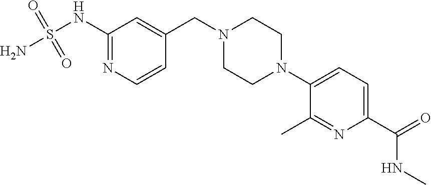 [SMILES: CNC(=O)c1ccc(N2CCN(Cc3ccnc(NS(N)(=O)=O)c3)CC2)c(C)n1] | N,6-dimethyl-5-(4-((2- (sulfamoylamino)pyridin-4- yl)methyl)piperazin-1-yl)picolinamide |
| 131 | 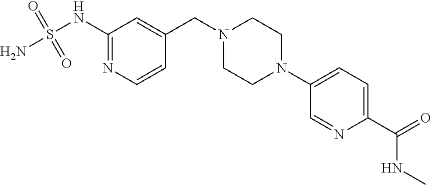 [SMILES: CNC(=O)c1ccc(N2CCN(Cc3ccnc(NS(N)(=O)=O)c3)CC2)cn1] | N-methyl-5-(4-((2- (sulfamoylamino)pyridin-4- yl)methyl)piperazin-1-yl)picolinamide |
| 132 | 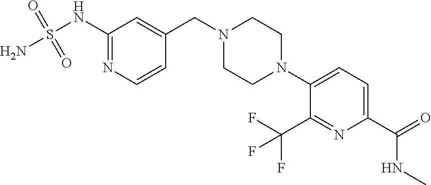 [SMILES: CNC(=O)c1ccc(N2CCN(Cc3ccnc(NS(N)(=O)=O)c3)CC2)c(C(F)(F)F)n1] | N-methyl-5-(4-((2- (sulfamoylamino)pyridin-4- yl)methyl)piperazin-1-yl)-6- (trifluoromethyl)picolinamide |
| 133 | 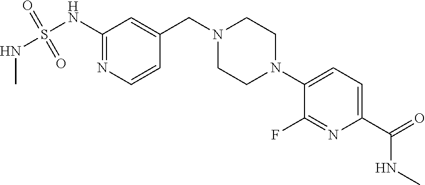 [SMILES: CNC(=O)c1ccc(N2CCN(Cc3ccnc(NS(=O)(=O)NC)c3)CC2)c(F)n1] | 6-fluoro-N-methyl-5-(4-((2-((N- methylsulfamoyl)amino)pyridin-4- yl)methyl)piperazin-1-yl)picolinamide |
| 134 | 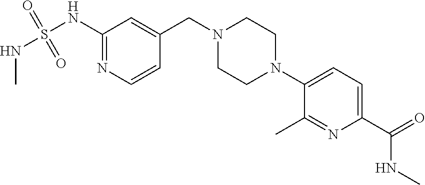 [SMILES: CNC(=O)c1ccc(N2CCN(Cc3ccnc(NS(=O)(=O)NC)c3)CC2)c(C)n1] | N,6-dimethyl-5-(4-((2-((N- methylsulfamoyl)amino)pyridin-4- yl)methyl)piperazin-1-yl)picolinamide |
| 135 | 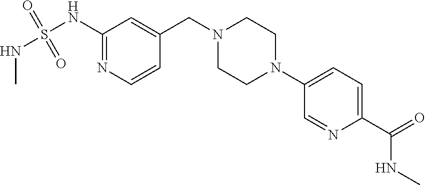 [SMILES: CNC(=O)c1ccc(N2CCN(Cc3ccnc(NS(=O)(=O)NC)c3)CC2)cn1] | N-methyl-5-(4-((2-((N- methylsulfamoyl)amino)pyridin-4- yl)methyl)piperazin-1-yl)picolinamide |
| 136 | 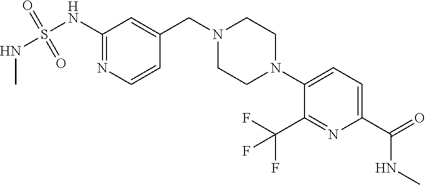 [SMILES: CNC(=O)c1ccc(N2CCN(Cc3ccnc(NS(=O)(=O)NC)c3)CC2)c(C(F)(F)F)n1] | N-methyl-5-(4-((2-((N- methylsulfamoyl)amino)pyridin-4- yl)methyl)piperazin-1-yl)-6- (trifluoromethyl)picolinamide |
| 137 | 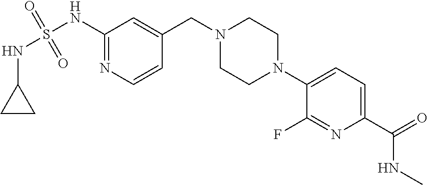 [SMILES: CNC(=O)c1ccc(N2CCN(Cc3ccnc(NS(=O)(=O)NC4CC4)c3)CC2)c(F)n1] | 5-(4-((2-((N- cyclopropylsulfamoyl)amino)pyridin-4- yl)methyl)piperazin-1-yl)-6-fluoro-N- methylpicolinamide |
| 138 | 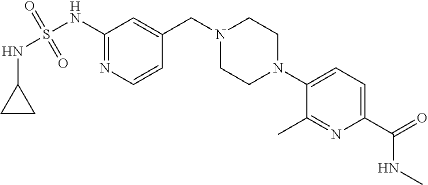 [SMILES: CNC(=O)c1ccc(N2CCN(Cc3ccnc(NS(=O)(=O)NC4CC4)c3)CC2)c(C)n1] | 5-(4-((2-((N- cyclopropylsulfamoyl)amino)pyridin-4- yl)methyl)piperazin-1-yl)-N,6- dimethylpicolinamide |
| 139 | 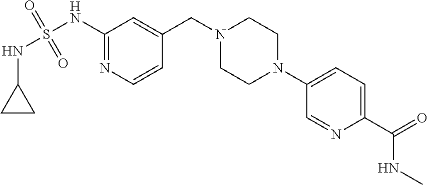 [SMILES: CNC(=O)c1ccc(N2CCN(Cc3ccnc(NS(=O)(=O)NC4CC4)c3)CC2)cn1] | 5-(4-((2-((N- cyclopropylsulfamoyl)amino)pyridin-4- yl)methyl)piperazin-1-yl)-N- methylpicolinamide |
| 140 | 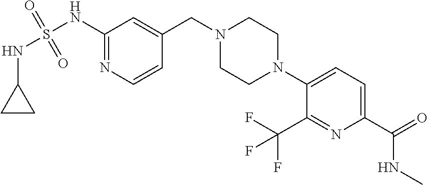 [SMILES: CNC(=O)c1ccc(N2CCN(Cc3ccnc(NS(=O)(=O)NC4CC4)c3)CC2)c(C(F)(F)F)n1] | 5-(4-((2-((N- cyclopropylsulfamoyl)amino)pyridin-4- yl)methyl)piperazin-1-yl)-N-methyl-6- (trifluoromethyl)picolinamide |
| 141 | 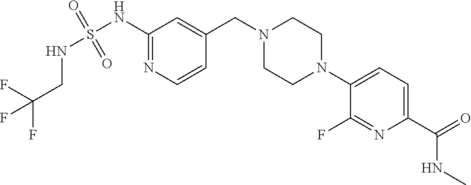 [SMILES: CNC(=O)c1ccc(N2CCN(Cc3ccnc(NS(=O)(=O)NCC(F)(F)F)c3)CC2)c(F)n1] | 6-fluoro-N-methyl-5-(4-((2-((N-(2,2,2- trifluoroetbyl)sulfamoyl)amino)pyridin-4- yl)methyl)piperazin-1-yl)picolinamide |
| 142 | 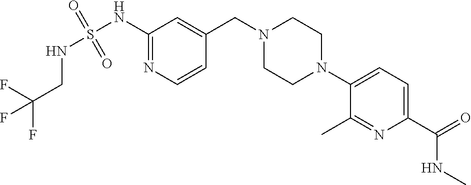 [SMILES: CNC(=O)c1ccc(N2CCN(Cc3ccnc(NS(=O)(=O)NCC(F)(F)F)c3)CC2)c(C)n1] | N,6-dimethyl-5-(4-((2-((N-(2,2,2- trifluoroethyl)sulfamoyl)amino)pyridin-4- yl)methyl)piperazin-1-yl)picolinamide |
| 143 | 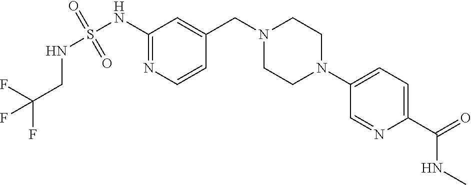 [SMILES: CNC(=O)c1ccc(N2CCN(Cc3ccnc(NS(=O)(=O)NCC(F)(F)F)c3)CC2)cn1] | N-methyl-5-(4-((2-((N-(2,2,2- trifluoroethyl)sulfamoyl)amino)pyridin-4- yl)methyl)piperazin-1-yl)picolinamide |
| 144 | 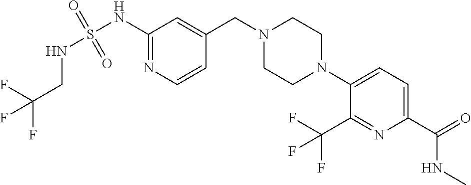 [SMILES: CNC(=O)c1ccc(N2CCN(Cc3ccnc(NS(=O)(=O)NCC(F)(F)F)c3)CC2)c(C(F)(F)F)n1] | N-methyl-5-(4-((2-((N-(2,2,2- trifluoroethyl)sulfamoyl)amino)pyridin-4- yl)methyl)piperazin-1-yl)-6- (trifluoromethyl)picolinamide |
| 145 | 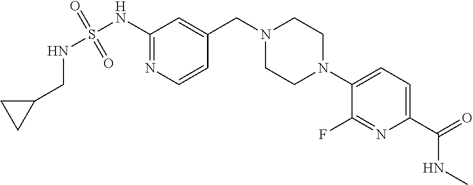 [SMILES: CNC(=O)c1ccc(N2CCN(Cc3ccnc(NS(=O)(=O)NCC4CC4)c3)CC2)c(F)n1] | 5-(4-((2-((N- (cyclopropylmethyl)sulfamoyl)amino) pyridin-4-yl)methyl)piperazin-1-yl)-6- fluoro-N-methylpicolinamide |
| 146 | 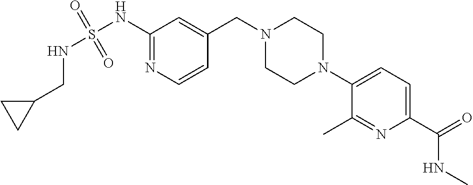 [SMILES: CNC(=O)c1ccc(N2CCN(Cc3ccnc(NS(=O)(=O)NCC4CC4)c3)CC2)c(C)n1] | 5-(4-((2-((N- (cyclopropylmethyl)sulfamoyl)amino) pyridin-4-yl)methyl)piperazin-1-yl)-N,6- dimethylpicolinamide |
| 147 | 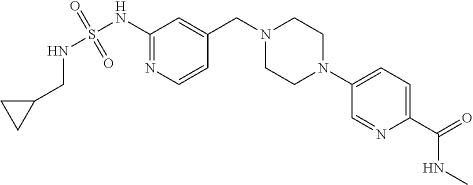 [SMILES: CNC(=O)c1ccc(N2CCN(Cc3ccnc(NS(=O)(=O)NCC4CC4)c3)CC2)cn1] | 5-(4-((2-((N- (cyclopropylmethyl)sulfamoyl)amino) pyridin-4-yl)methyl)piperazin-1-yl)-N- methylpicolinamide |
| 148 | 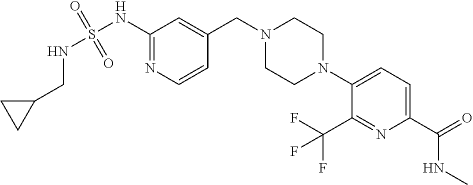 [SMILES: CNC(=O)c1ccc(N2CCN(Cc3ccnc(NS(=O)(=O)NCC4CC4)c3)CC2)c(C(F)(F)F)n1] | 5-(4-((2-((N- (cyclopropylmethyl)sulfamoyl)amino) pyridin-4-yl)methyl)piperazin-1-yl)-N- methyl-6-(trifluoromethyl)picolinamide |
| 149 | 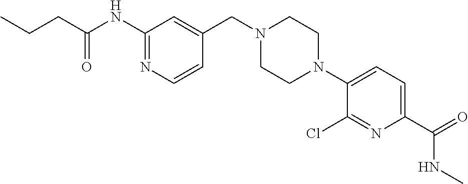 [SMILES: CCCC(=O)Nc1cc(CN2CCN(c3ccc(C(=O)NC)nc3Cl)CC2)ccn1] | 5-(4-((2-butyramidopyridin-4- yl)methyl)piperazin-1-yl)-6-chloro-N- methylpicolinamide |
| 150 | 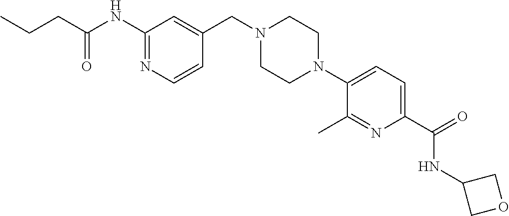 [SMILES: CCCC(=O)Nc1cc(CN2CCN(c3ccc(C(=O)NC4COC4)nc3C)CC2)ccn1] | 5-(4-((2-butyramidopyridin-4- yl)methyl)piperazin-1-yl)-6-methyl-N- (oxetan-3-yl)picolinamide |
| 151 | 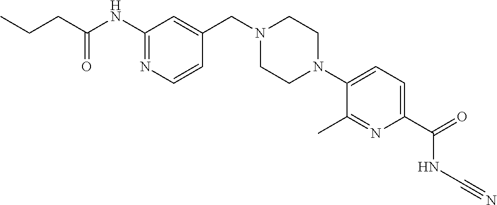 [SMILES: CCCC(=O)Nc1cc(CN2CCN(c3ccc(C(=O)NC#N)nc3C)CC2)ccn1] | 5-(4-((2-butyramidopyridin-4- yl)methyl)piperazin-1-yl)-N-cyano-6- methylpicolinamide |
| 152 | 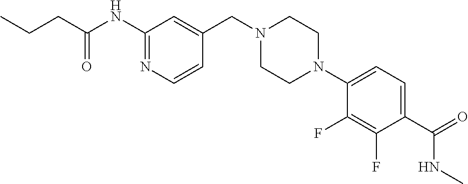 [SMILES: CCCC(=O)Nc1cc(CN2CCN(c3ccc(C(=O)NC)c(F)c3F)CC2)ccn1] | 4-(4-((2-butyramidopyridin-4- yl)methyl)piperazin-1-yl)-2,3-difluoro-N- methylbenzamide |
| 153 | 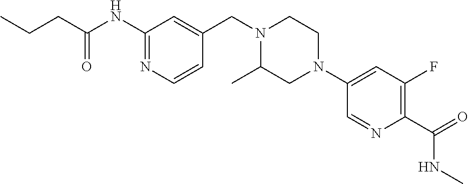 [SMILES: CCCC(=O)Nc1cc(CN2CCN(c3cnc(C(=O)NC)c(F)c3)CC2C)ccn1] | 5-(4-((2-butyramidopyridin-4-yl)methyl)- 3-methylpiperazin-1-yl)-3-fluoro-N- methylpicolinamide |
| 154 | 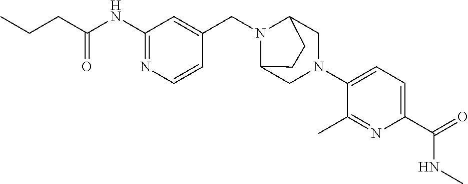 [SMILES: CCCC(=O)Nc1cc(CN2C3CCC2CN(c2ccc(C(=O)NC)nc2C)C3)ccn1] | 5-(8-((2-butyramidopyridin-4-yl)methyl)- 3,8-diazabicyclo[3.2.1]octan-3-yl)-N,6- dimethylpicolinamide |
| 155 | 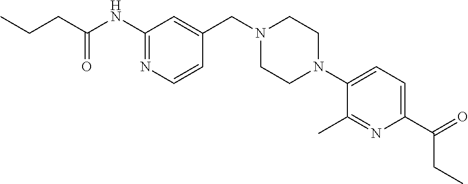 [SMILES: CCCC(=O)Nc1cc(CN2CCN(c3ccc(C(=O)CC)nc3C)CC2)ccn1] | N-(4-((4-(2-methyl-6-propionylpyridin-3- yl)piperazin-1-yl)methyl)pyridin-2- yl)butyramide |
| 156 | 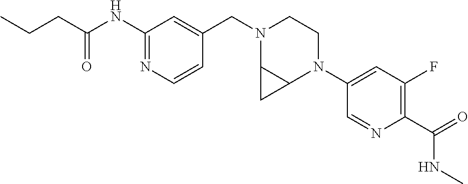 [SMILES: CCCC(=O)Nc1cc(CN2CCN(c3cnc(C(=O)NC)c(F)c3)C3CC32)ccn1] | S-(5-((2-butyramidopyridin-4-yl)methyl)- 2,5-diazabicyclo[4.1.0]heptan-2-yl)-3- fluoro-N-methylpicolinamide |
| 157 | 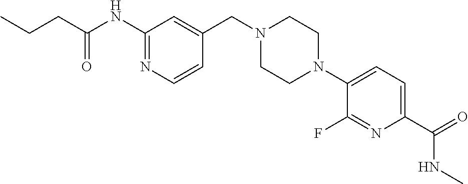 [SMILES: CCCC(=O)Nc1cc(CN2CCN(c3ccc(C(=O)NC)nc3F)CC2)ccn1] | 5-(4-((2-butyramidopyridin-4- yl)methyl)piperazin-1-yl)-6-fluoro-N- methylpicolinamide |
| 158 | 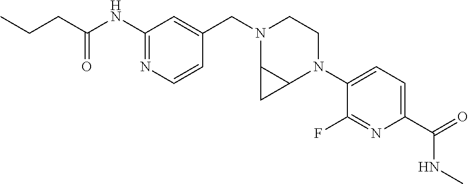 [SMILES: CCCC(=O)Nc1cc(CN2CCN(c3ccc(C(=O)NC)nc3F)C3CC32)ccn1] | 5-(5-((2-butyramidopyridin-4-yl)methyl)- 2,5-diazabicyclo[4.1.0]heptan-2-yl)-6- fluoro-N-methylpicolinamide |
| 159 | 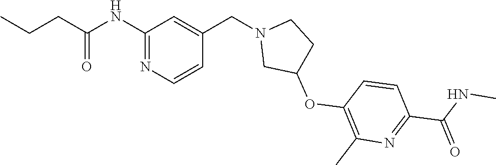 [SMILES: CCCC(=O)Nc1cc(CN2CCC(Oc3ccc(C(=O)NC)nc3C)C2)ccn1] | 5-((1-((2-butyramidopyridin-4- yl)methyl)pyrrolidin-3-yl)oxy)-N,6- dimethylpicolinamide |
| 160 | 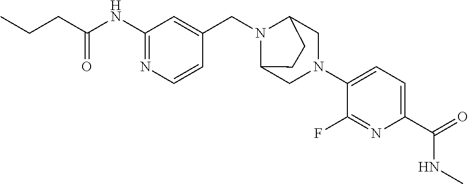 [SMILES: CCCC(=O)Nc1cc(CN2C3CCC2CN(c2ccc(C(=O)NC)nc2F)C3)ccn1] | 5-(8-((2-butyramidopyridin-4-yl)methyl)- 3,8-diazabicyclo[3.2.1]octan-3-yl)-6- fluoro-N-methylpicolinamide |
| 161 | 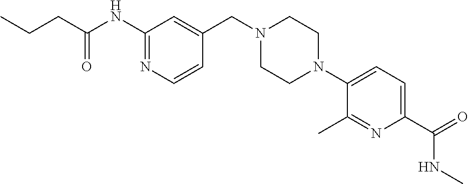 [SMILES: CCCC(=O)Nc1cc(CN2CCN(c3ccc(C(=O)NC)nc3C)CC2)ccn1] | 5-(4-((2-butyramidopyridin-4- yl)methyl)piperazin-1-yl)-N,6- dimethylpicolinamide |
| 162 | 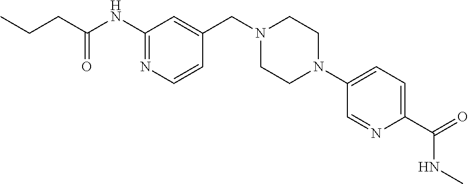 [SMILES: CCCC(=O)Nc1cc(CN2CCN(c3ccc(C(=O)NC)nc3)CC2)ccn1] | 5-(4-((2-butyramidopyridin-4- yl)methyl)piperazin-1-yl)-N- methylpicolinamide |
| 163 | 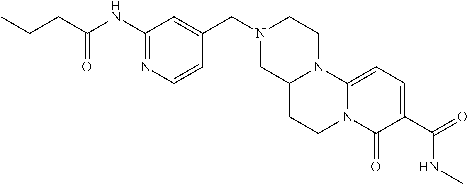 [SMILES: CCCC(=O)Nc1cc(CN2CCN3c4ccc(C(=O)NC)c(=O)n4CCC3C2)ccn1] | 3-((2-butyramidopyridin-4-yl)methyl)-N- methyl-8-oxo-2,3,4,4a,5,6-hexahydro- 1H,8H-pyrazino[1,2-c]pyrido[1,2- a]pyrimidine-9-carboxamide |
| 164 | 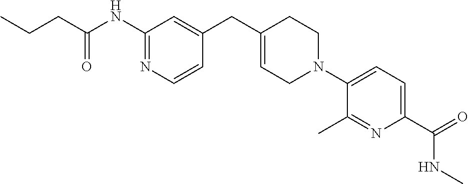 [SMILES: CCCC(=O)Nc1cc(CC2=CCN(c3ccc(C(=O)NC)nc3C)CC2)ccn1] | 4-((2-butyramidopyridin-4-yl)methyl)- N,2′-dimethyl-3,6-dihydro-2H-[1,3′- bipyridine]-6′-carboxamide |
| 165 | 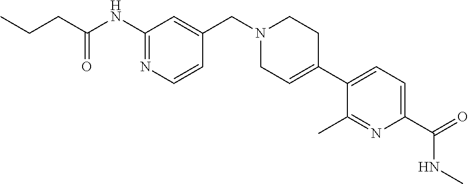 [SMILES: CCCC(=O)Nc1cc(CN2CC=C(c3ccc(C(=O)NC)nc3C)CC2)ccn1] | 1′-((2-butyramidopyridin-4-yl)methyl)- N,2-dimethyl-1′,2′,3′,6′-tetrahydro-[3,4′- bipyridine]-6-carboxamide |
| 166 | 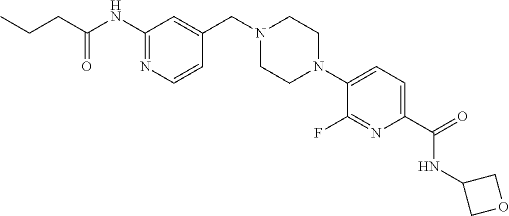 [SMILES: CCCC(=O)Nc1cc(CN2CCN(c3ccc(C(=O)NC4COC4)nc3F)CC2)ccn1] | 5-(4-((2-butyramidopyridin-4- yl)methyl)piperazin-1-yl)-6-fluoro-N- (oxetan-3-yl)picolinamide |
| 167 | 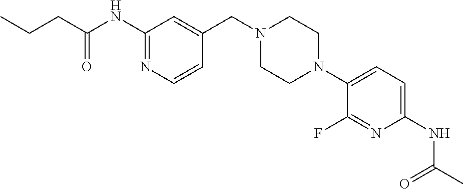 [SMILES: CCCC(=O)Nc1cc(CN2CCN(c3ccc(NC(C)=O)nc3F)CC2)ccn1] | N-(4-((4-(6-acetamido-2-fluoropyridin-3- yl)piperazin-1-yl)methyl)pyridin-2- yl)butyramide |
| 168 | 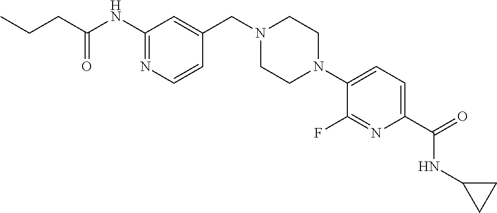 [SMILES: CCCC(=O)Nc1cc(CN2CCN(c3ccc(C(=O)NC4CC4)nc3F)CC2)ccn1] | 5-(4-((2-butyramidopyridin-4- yl)methyl)piperazin-1-yl)-N-cyclopropyl- 6-fluoropicolinamide |
| 169 | 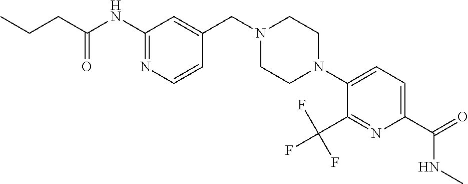 [SMILES: CCCC(=O)Nc1cc(CN2CCN(c3ccc(C(=O)NC)nc3C(F)(F)F)CC2)ccn1] | 5-(4-((2-butyramidopyridin-4- yl)methyl)piperazin-1-yl)-N-methyl-6- (trifluoromethyl)picolinamide |
| 170 | 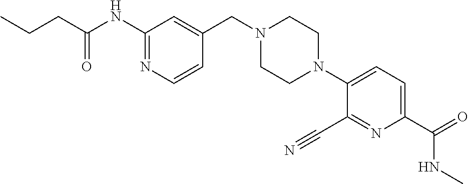 [SMILES: CCCC(=O)Nc1cc(CN2CCN(c3ccc(C(=O)NC)nc3C#N)CC2)ccn1] | 5-(4-((2-butyramidopyridin-4- yl)methyl)piperazin-1-yl)-6-cyano-N- methylpicolinamide |
| 171 | 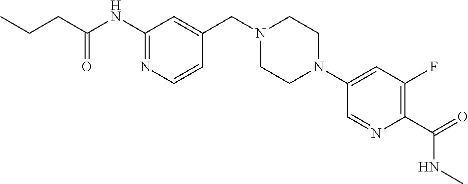 [SMILES: CCCC(=O)Nc1cc(CN2CCN(c3cnc(C(=O)NC)c(F)c3)CC2)ccn1] | 5-(4-((2-butyramidopyridin-4- yl)methyl)piperazin-1-yl)-3-fluoro-N- methylpicolinamide |
| 172 | 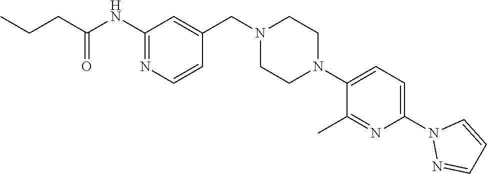 [SMILES: CCCC(=O)Nc1cc(CN2CCN(c3ccc(-n4cccn4)nc3C)CC2)ccn1] | N-(4-((4-(2-methyl-6-(1H-pyrazol-1- yl)pyridin-3-yl)piperazin-1- yl)methyl)pyridin-2-yl)butyramide |
| 173 | 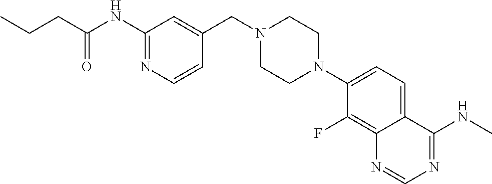 [SMILES: CCCC(=O)Nc1cc(CN2CCN(c3ccc4c(NC)ncnc4c3F)CC2)ccn1] | N-(4-((4-(8-fluoro-4- (methylamino)quinazolin-7-yl)piperazin- 1-yl)methyl)pyridin-2-yl)butyramide |
| 174 | 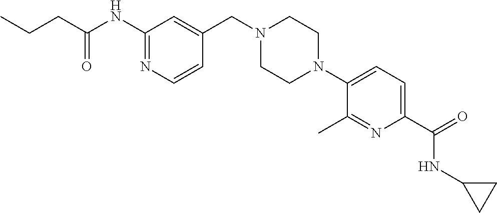 [SMILES: CCCC(=O)Nc1cc(CN2CCN(c3ccc(C(=O)NC4CC4)nc3C)CC2)ccn1] | 5-(4-((2-butyramidopyridin-4- yl)methyl)piperazin-1-yl)-N-cyclopropyl- 6-methylpicolinamide |
| 175 | 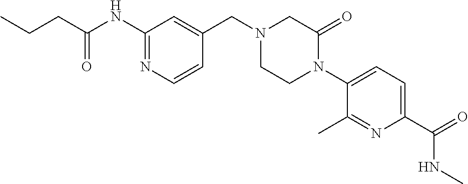 [SMILES: CCCC(=O)Nc1cc(CN2CCN(c3ccc(C(=O)NC)nc3C)C(=O)C2)ccn1] | 5-(4-((2-butyramidopyridin-4-yl)methyl)- 2-oxopiperazin-1-yl)-N,6- dimethylpicolinamide |
| 176 | 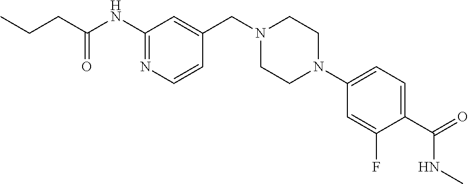 [SMILES: CCCC(=O)Nc1cc(CN2CCN(c3ccc(C(=O)NC)c(F)c3)CC2)ccn1] | 4-(4-((2-butyramidopyridin-4- yl)methyl)piperazin-1-yl)-2-fluoro-N- methylbenzamide |
| 177 | 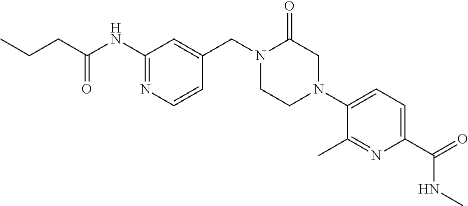 [SMILES: CCCC(=O)Nc1cc(CN2CCN(c3ccc(C(=O)NC)nc3C)CC2=O)ccn1] | 5-(4-((2-butyramidopyridin-4-yl)methyl)- 3-oxopiperazin-1-yl)-N,6- dimethylpicolinamide |
| 178 | 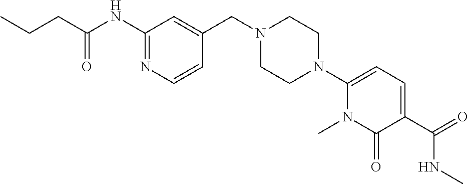 [SMILES: CCCC(=O)Nc1cc(CN2CCN(c3ccc(C(=O)NC)c(=O)n3C)CC2)ccn1] | 6-(4-((2-butyramidopyridin-4- yl)methyl)piperazin-1-yl)-N,1-dimethyl- 2-oxo-1,2-dihydropyridine-3- carboxamide |
| 179 | 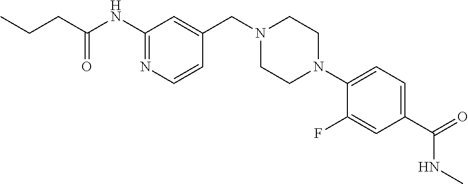 [SMILES: CCCC(=O)Nc1cc(CN2CCN(c3ccc(C(=O)NC)cc3F)CC2)ccn1] | 4-(4-((2-butyramidopyridin-4- yl)methyl)piperazin-1-yl)-3-fluoro-N- methylbenzamide |
| 180 | 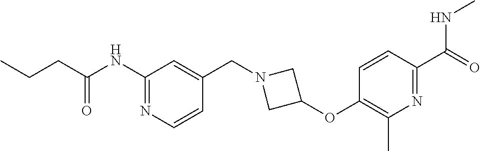 [SMILES: CCCC(=O)Nc1cc(CN2CC(Oc3ccc(C(=O)NC)nc3C)C2)ccn1] | S-((1-((2-butyramidopyridin-4- yl)methyl)azetidin-3-yl)oxy)-N,6- dimethylpicolinamide |
| 181 | 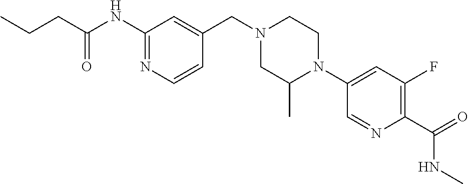 [SMILES: CCCC(=O)Nc1cc(CN2CCN(c3cnc(C(=O)NC)c(F)c3)C(C)C2)ccn1] | 5-(4-((2-butyramidopyridin-4-yl)methyl)- 2-methylpiperazin-1-yl)-3-fluoro-N- methylpicolinamide |
| 182 | 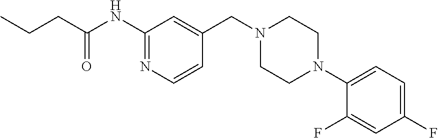 [SMILES: CCCC(=O)Nc1cc(CN2CCN(c3ccc(F)cc3F)CC2)ccn1] | N-(4-((4-(2,4-difluorophenyl)piperazin-1- yl)methyl)pyridin-2-yl)butyramide |
| 183 | 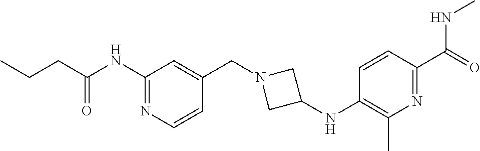 [SMILES: CCCC(=O)Nc1cc(CN2CC(Nc3ccc(C(=O)NC)nc3C)C2)ccn1] | 5-((1-((2-butyramidopyridin-4- yl)methyl)azetidin-3-yl)amino)-N,6- dimethylpicolinamide |
| 184 | 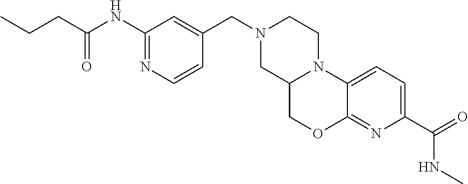 [SMILES: CCCC(=O)Nc1cc(CN2CCN3c4ccc(C(=O)NC)nc4OCC3C2)ccn1] | 3-((2-butyramidopyridin-4-yl)methyl)-N- methyl-1,2,3,4,4a,5- hexahydropyrazino[1,2-d]pyrido[2,3- b][1,4]oxazine-8-carboxamide |
| 185 | 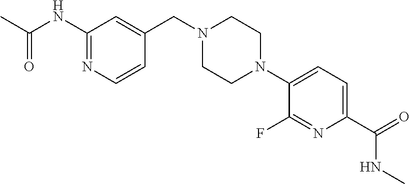 [SMILES: CNC(=O)c1ccc(N2CCN(Cc3ccnc(NC(C)=O)c3)CC2)c(F)n1] | 5-(4-((2-acetamidopyridin-4- yl)methyl)piperazin-1-yl)-6-fluoro-N- methylpicolinamide |
| 186 | 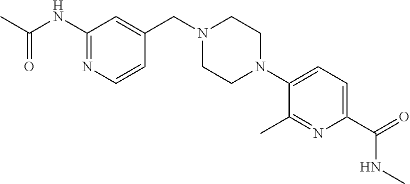 [SMILES: CNC(=O)c1ccc(N2CCN(Cc3ccnc(NC(C)=O)c3)CC2)c(C)n1] | 5-(4-((2-acetamidopyridin-4- yl)methyl)piperazin-1-yl)-N,6- dimethylpicolinamide |
| 187 | 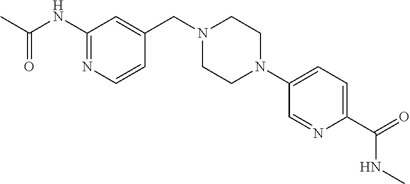 [SMILES: CNC(=O)c1ccc(N2CCN(Cc3ccnc(NC(C)=O)c3)CC2)cn1] | 5-(4-((2-acetamidopyridin-4- yl)methyl)piperazin-1-yl)-N- methylpicolinamide |
| 188 | 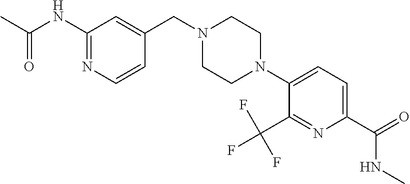 [SMILES: CNC(=O)c1ccc(N2CCN(Cc3ccnc(NC(C)=O)c3)CC2)c(C(F)(F)F)n1] | 5-(4-((2-acetamidopyridin-4- yl)methyl)piperazin-1-yl)-N-methyl-6- (trifluoromethyl)picolinamide |
| 189 | 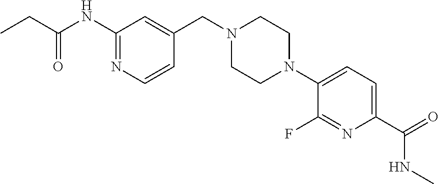 [SMILES: CCC(=O)Nc1cc(CN2CCN(c3ccc(C(=O)NC)nc3F)CC2)ccn1] | 6-fluoro-N-methyl-5-(4-((2- propionamidopyridin-4- yl)methyl)piperazin-1-yl)picolinamide |
| 190 | 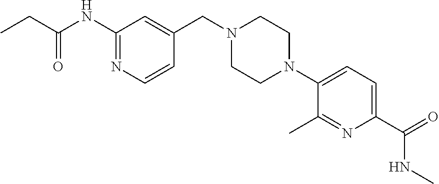 [SMILES: CCC(=O)Nc1cc(CN2CCN(c3ccc(C(=O)NC)nc3C)CC2)ccn1] | N,6-dimethyl-5-(4-((2- propionamidopyridin-4- yl)methyl)piperazin-1-yl)picolinamide |
| 191 | 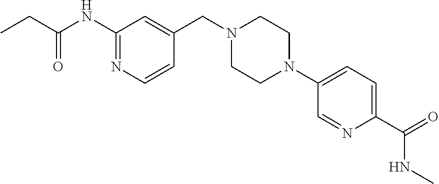 [SMILES: CCC(=O)Nc1cc(CN2CCN(c3ccc(C(=O)NC)nc3)CC2)ccn1] | N-methyl-5-(4-((2-propionamidopyridin- 4-yl)methyl)piperazin-1-yl)picolinamide |
| 192 | 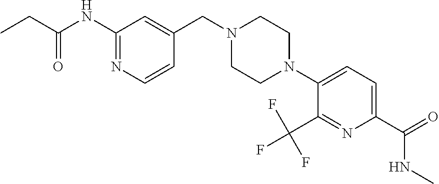 [SMILES: CCC(=O)Nc1cc(CN2CCN(c3ccc(C(=O)NC)nc3C(F)(F)F)CC2)ccn1] | N-methyl-5-(4-((2-propionamidopyridin- 4-yl)methyl)piperazin-1-yl)-6- (trifluoromethyl)picolinamide |
| 193 | 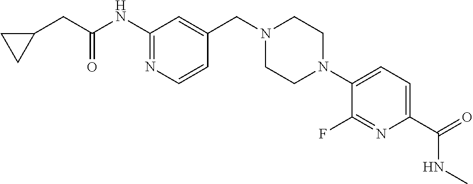 [SMILES: CNC(=O)c1ccc(N2CCN(Cc3ccnc(NC(=O)CC4CC4)c3)CC2)c(F)n1] | 5-(4-((2-(2- cyclopropylacetamido)pyridin-4- yl)methyl)piperazin-1-yl)-6-fluoro-N- methylpicolinamide |
| 194 | 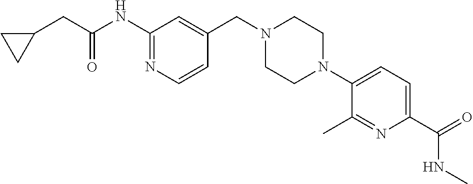 [SMILES: CNC(=O)c1ccc(N2CCN(Cc3ccnc(NC(=O)CC4CC4)c3)CC2)c(C)n1] | 5-(4-((2-(2- cyclopropylacetamido)pyridin-4- yl)methyl)piperazin-1-yl)-N,6- dimethylpicolinamide |
| 195 | 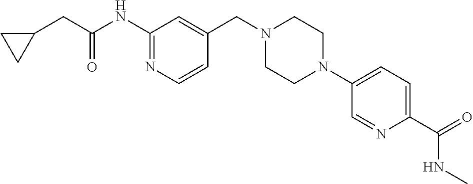 [SMILES: CNC(=O)c1ccc(N2CCN(Cc3ccnc(NC(=O)CC4CC4)c3)CC2)cn1] | 5-(4-((2-(2- cyclopropylacetamido)pyridin-4- yl)methyl)piperazin-1-yl)-N- methylpicolinamide |
| 196 | 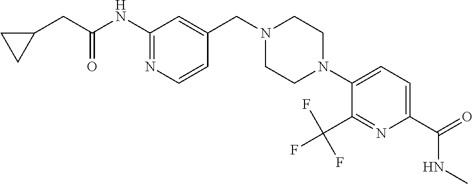 [SMILES: CNC(=O)c1ccc(N2CCN(Cc3ccnc(NC(=O)CC4CC4)c3)CC2)c(C(F)(F)F)n1] | 5-(4-((2-(2- cyclopropylacetamido)pyridin-4- yl)methyl)piperazin-1-yl)-N-methyl-6- (trifluoromethyl)picolinamide |
| 197 | 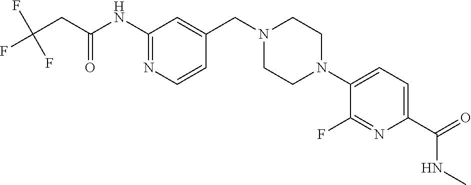 [SMILES: CNC(=O)c1ccc(N2CCN(Cc3ccnc(NC(=O)CC(F)(F)F)c3)CC2)c(F)n1] | 6-fluoro-N-methyl-5-(4-((2-(3,3,3- trifluoropropanamido)pyridin-4- yl)methyl)piperazin-1-yl)picolinamide |
| 198 | 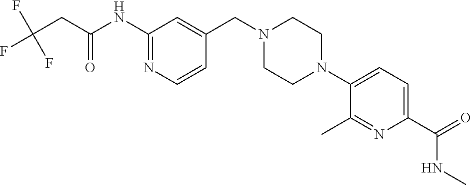 [SMILES: CNC(=O)c1ccc(N2CCN(Cc3ccnc(NC(=O)CC(F)(F)F)c3)CC2)c(C)n1] | N,6-dimethyl-5-(4-((2-(3,3,3- trifluoropropanamido)pyridin-4- yl)methyl)piperazin-1-yl)picolinamide |
| 199 | 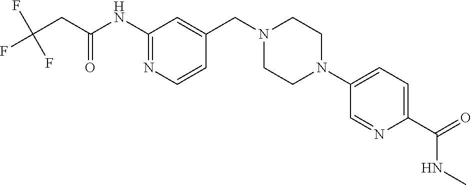 [SMILES: CNC(=O)c1ccc(N2CCN(Cc3ccnc(NC(=O)CC(F)(F)F)c3)CC2)cn1] | N-methyl-5-(4-((2-(3,3,3- trifluoropropanamido)pyridin-4- yl)methyl)piperazin-1-yl)picolinamide |
| 200 | 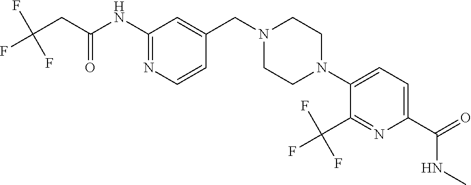 [SMILES: CNC(=O)c1ccc(N2CCN(Cc3ccnc(NC(=O)CC(F)(F)F)c3)CC2)c(C(F)(F)F)n1] | N-methyl-6-(trifluoromethyl)-5-(4-((2- (3,3,3-trifluoropropanamido)pyridin-4- yl)methyl)piperazin-1-yl)picolinamide |
| 201 | 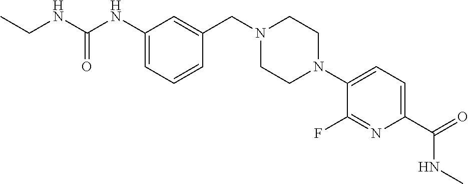 [SMILES: CCNC(=O)Nc1cccc(CN2CCN(c3ccc(C(=O)NC)nc3F)CC2)c1] | 5-(4-(3-(3-ethylureido)benzyl)piperazin- 1-yl)-6-fluoro-N-methylpicolinamide |
| 202 | 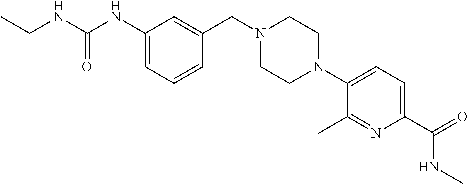 [SMILES: CCNC(=O)Nc1cccc(CN2CCN(c3ccc(C(=O)NC)nc3C)CC2)c1] | 5-(4-(3-(3-ethylureido)benzyl)piperazin- 1-yl)-N,6-dimethylpicolinamide |
| 203 | 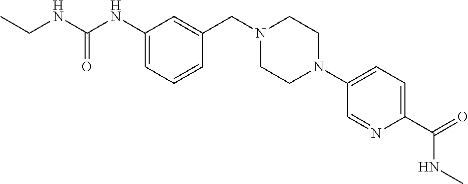 [SMILES: CCNC(=O)Nc1cccc(CN2CCN(c3ccc(C(=O)NC)nc3)CC2)c1] | 5-(4-(3-(3-ethylureido)benzyl)piperazin- 1-yl)-N-methylpicolinamide |
| 204 | 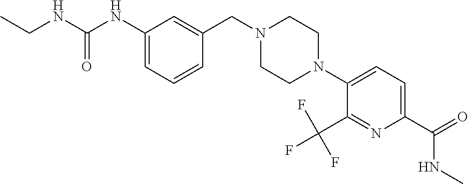 [SMILES: CCNC(=O)Nc1cccc(CN2CCN(c3ccc(C(=O)NC)nc3C(F)(F)F)CC2)c1] | 5-(4-(3-(3-ethylureido)benzyl)piperazin- 1-yl)-N-methyl-6- (trifluoromethyl)picolinamide |
| 205 | 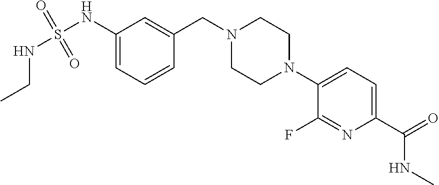 [SMILES: CCNS(=O)(=O)Nc1cccc(CN2CCN(c3ccc(C(=O)NC)nc3F)CC2)c1] | 5-(4-(3-((N- ethylsulfamoyl)amino)benzyl)piperazin- 1-yl)-6-fluoro-N-methylpicolinamide |
| 206 | 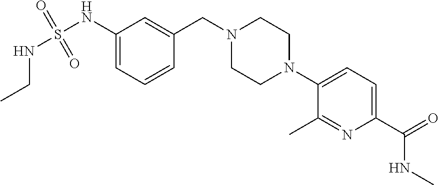 [SMILES: CCNS(=O)(=O)Nc1cccc(CN2CCN(c3ccc(C(=O)NC)nc3C)CC2)c1] | 5-(4-(3-((N- ethylsulfamoyl)amino)benzyl)piperazin- 1-yl)-N,6-dimethylpicolinamide |
| 207 | 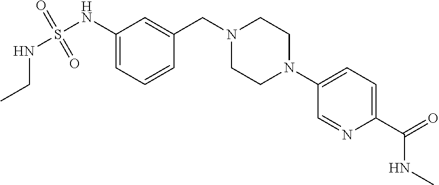 [SMILES: CCNS(=O)(=O)Nc1cccc(CN2CCN(c3ccc(C(=O)NC)nc3)CC2)c1] | 5-(4-(3-((N- ethylsulfamoyl)amino)benzyl)piperazin- 1-yl)-N-methylpicolinamide |
| 208 | 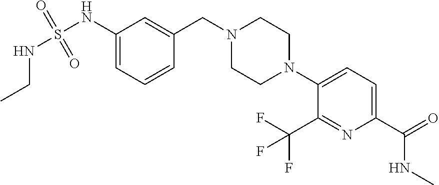 [SMILES: CCNS(=O)(=O)Nc1cccc(CN2CCN(c3ccc(C(=O)NC)nc3C(F)(F)F)CC2)c1] | 5-(4-(3-((N- ethylsulfamoyl)amino)benzyl)piperazin- 1-yl)-N-methyl-6- (trifluoromethyl)picolinamide |
| 209 | 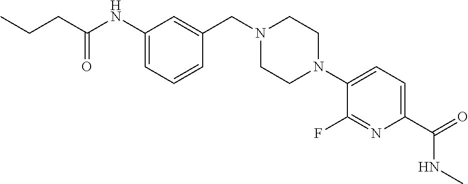 [SMILES: CCCC(=O)Nc1cccc(CN2CCN(c3ccc(C(=O)NC)nc3F)CC2)c1] | 5-(4-(3-butyramidobenzyl)piperazin-1- yl)-6-fluoro-N-methylpicolinamide |
| 210 | 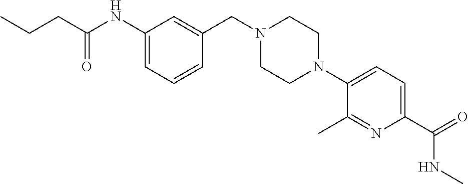 [SMILES: CCCC(=O)Nc1cccc(CN2CCN(c3ccc(C(=O)NC)nc3C)CC2)c1] | 5-(4-(3-butyramidobenzyl)piperazin-1- yl)-N,6-dimethylpicolinamide |
| 211 | 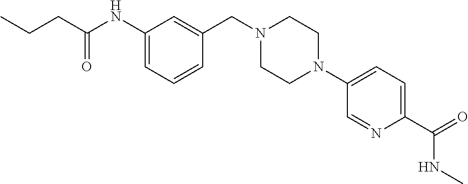 [SMILES: CCCC(=O)Nc1cccc(CN2CCN(c3ccc(C(=O)NC)nc3)CC2)c1] | 5-(4-(3-butyramidobenzyl)piperazin-1- yl)-N-methylpicolinamide |
| 212 | 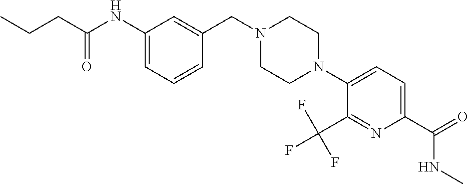 [SMILES: CCCC(=O)Nc1cccc(CN2CCN(c3ccc(C(=O)NC)nc3C(F)(F)F)CC2)c1] | 5-(4-(3-butyramidobenzyl)piperazin-1- yl)-N-methyl-6- (trifluoromethyl)picolinamide |
| 213 | 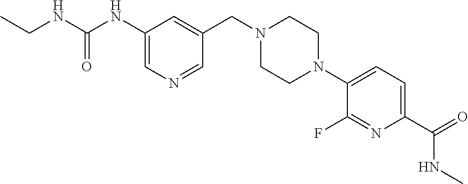 [SMILES: CCNC(=O)Nc1cncc(CN2CCN(c3ccc(C(=O)NC)nc3F)CC2)c1] | 5-(4-((5-(3-ethylureido)pyridin-3- yl)methyl)piperazin-1-yl)-6-fluoro-N- methylpicolinamide |
| 214 | 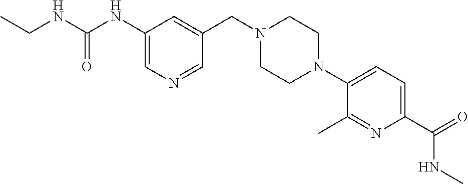 [SMILES: CCNC(=O)Nc1cncc(CN2CCN(c3ccc(C(=O)NC)nc3C)CC2)c1] | 5-(4-((5-(3-ethylureido)pyridin-3- yl)methyl)piperazin-1-yl)-N,6- dimethylpicolinamide |
| 215 | 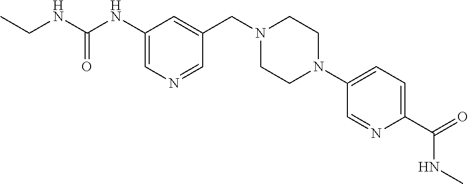 [SMILES: CCNC(=O)Nc1cncc(CN2CCN(c3ccc(C(=O)NC)nc3)CC2)c1] | 5-(4-((5-(3-ethylureido)pyridin-3- yl)methyl)piperazin-1-yl)-N- methylpicolinamide |
| 216 | 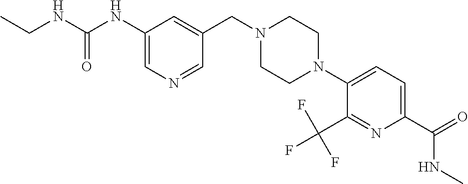 [SMILES: CCNC(=O)Nc1cncc(CN2CCN(c3ccc(C(=O)NC)nc3C(F)(F)F)CC2)c1] | 5-(4-((5-(3-ethylureido)pyridin-3- yl)methyl)piperazin-1-yl)-N-methyl-6- (trifluoromethyl)picolinamide |
| 217 | 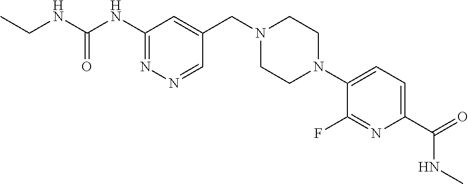 [SMILES: CCNC(=O)Nc1cc(CN2CCN(c3ccc(C(=O)NC)nc3F)CC2)cnn1] | 5-(4-((6-(3-ethylureido)pyridazin-4- yl)methyl)piperazin-1-yl)-6-fluoro-N- methylpicolinamide |
| 218 | 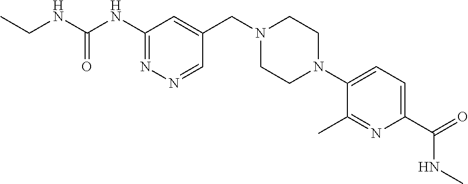 [SMILES: CCNC(=O)Nc1cc(CN2CCN(c3ccc(C(=O)NC)nc3C)CC2)cnn1] | 5-(4-((6-(3-ethylureido)pyridazin-4- yl)methyl)piperazin-1-yl)-N,6- dimethylpicolinamide |
| 219 | 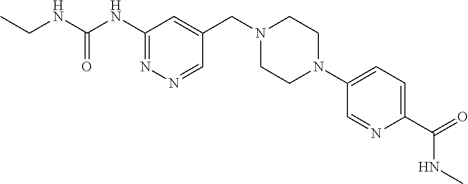 [SMILES: CCNC(=O)Nc1cc(CN2CCN(c3ccc(C(=O)NC)nc3)CC2)cnn1] | 5-(4-((6-(3-ethylureido)pyridazin-4- yl)methyl)piperazin-1-yl)-N- methylpicolinamide |
| 220 | 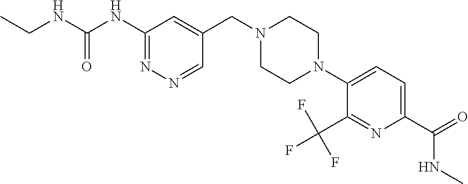 [SMILES: CCNC(=O)Nc1cc(CN2CCN(c3ccc(C(=O)NC)nc3C(F)(F)F)CC2)cnn1] | 5-(4-((6-(3-ethylureido)pyridazin-4- yl)methyl)piperazin-1-yl)-N-methyl-6- (trifluoromethyl)picolinamide |
| 221 | 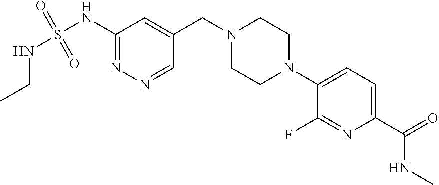 [SMILES: CCNS(=O)(=O)Nc1cc(CN2CCN(c3ccc(C(=O)NC)nc3F)CC2)cnn1] | 5-(4-((6-((N- ethylsulfamoyl)amino)pyridazin-4- yl)methyl)piperazin-1-yl)-6-fluoro-N- methylpicolinamide |
| 222 | 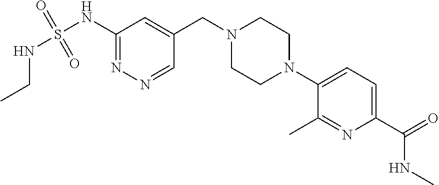 [SMILES: CCNS(=O)(=O)Nc1cc(CN2CCN(c3ccc(C(=O)NC)nc3C)CC2)cnn1] | 5-(4-((6-((N- ethylsulfamoyl)amino)pyridazin-4- yl)methyl)piperazin-1-yl)-N,6- dimethylpicolinamide |
| 223 | 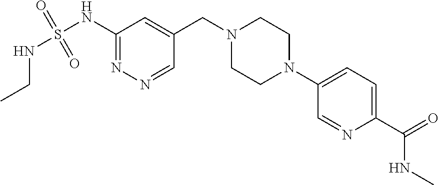 [SMILES: CCNS(=O)(=O)Nc1cc(CN2CCN(c3ccc(C(=O)NC)nc3)CC2)cnn1] | 5-(4-((6-((N- ethylsulfamoyl)amino)pyridazin-4- yl)methyl)piperazin-1-yl)-N- methylpicolinamide |
| 224 | 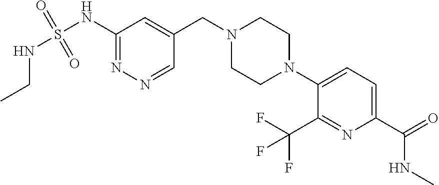 [SMILES: CCNS(=O)(=O)Nc1cc(CN2CCN(c3ccc(C(=O)NC)nc3C(F)(F)F)CC2)cnn1] | 5-(4-((6-((N- ethylsulfamoyl)amino)pyridazin-4- yl)methyl)piperazin-1-yl)-N-methyl-6- (trifluoromethyl)picolinamide |
| 225 | 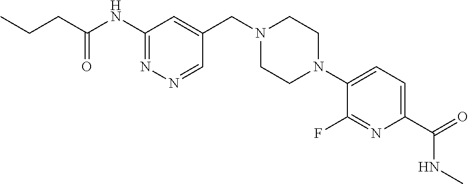 [SMILES: CCCC(=O)Nc1cc(CN2CCN(c3ccc(C(=O)NC)nc3F)CC2)cnn1] | 5-(4-((6-butyramidopyridazin-4- yl)methyl)piperazin-1-yl)-6-fluoro-N- methylpicolinamide |
| 226 | 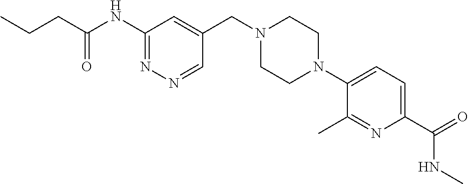 [SMILES: CCCC(=O)Nc1cc(CN2CCN(c3ccc(C(=O)NC)nc3C)CC2)cnn1] | 5-(4-((6-butyramidopyridazin-4- yl)methyl)piperazin-1-yl)-N,6- dimethylpicolinamide |
| 227 | 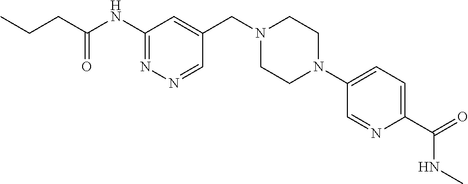 [SMILES: CCCC(=O)Nc1cc(CN2CCN(c3ccc(C(=O)NC)nc3)CC2)cnn1] | 5-(4-((6-butyramidopyridazin-4- yl)methyl)piperazin-1-yl)-N- methylpicolinamide |
| 228 | 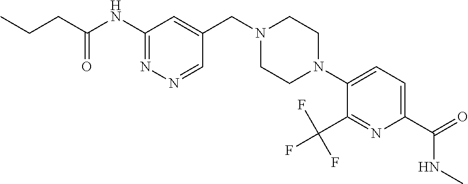 [SMILES: CCCC(=O)Nc1cc(CN2CCN(c3ccc(C(=O)NC)nc3C(F)(F)F)CC2)cnn1] | 5-(4-((6-butyramidopyridazin-4- yl)methyl)piperazin-1-yl)-N-methyl-6- (trifluoromethyl)picolinamide |
| 229 | 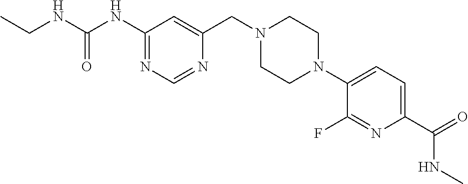 [SMILES: CCNC(=O)Nc1cc(CN2CCN(c3ccc(C(=O)NC)nc3F)CC2)ncn1] | 5-(4-((6-(3-ethylureido)pyrimidin-4- yl)methyl)piperazin-1-yl)-6-fluoro-N- methylpicolinamide |
| 230 | 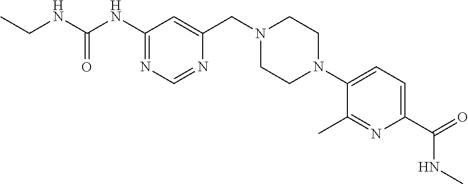 [SMILES: CCNC(=O)Nc1cc(CN2CCN(c3ccc(C(=O)NC)nc3C)CC2)ncn1] | 5-(4-((6-(3-ethylureido)pyrimidin-4- yl)methyl)piperazin-1-yl)-N,6- dimethylpicolinamide |
| 231 | 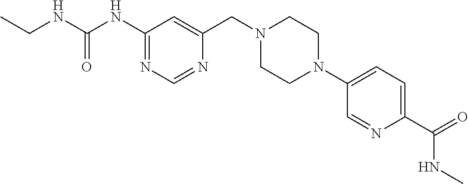 [SMILES: CCNC(=O)Nc1cc(CN2CCN(c3ccc(C(=O)NC)nc3)CC2)ncn1] | 5-(4-((6-(3-ethylureido)pyrimidin-4- yl)methyl)piperazin-1-yl)-N- methylpicolinamide |
| 232 | 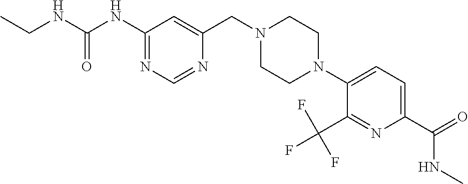 [SMILES: CCNC(=O)Nc1cc(CN2CCN(c3ccc(C(=O)NC)nc3C(F)(F)F)CC2)ncn1] | 5-(4-((6-(3-ethylureido)pyrimidin-4- yl)methyl)piperazin-1-yl)-N-methyl-6- (trifluoromethyl)picolinamide |
| 233 | 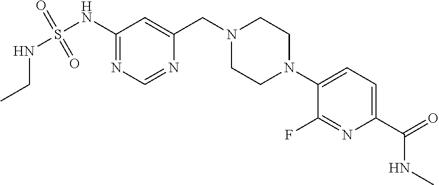 [SMILES: CCNS(=O)(=O)Nc1cc(CN2CCN(c3ccc(C(=O)NC)nc3F)CC2)ncn1] | 5-(4-((6-((N- ethylsulfamoyl)amino)pyrimidin-4- yl)methyl)piperazin-1-yl)-6-fluoro-N- methylpicolinamide |
| 234 | 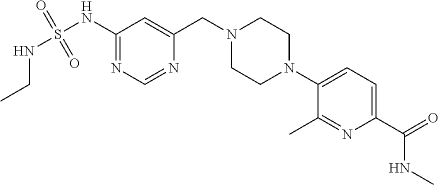 [SMILES: CCNS(=O)(=O)Nc1cc(CN2CCN(c3ccc(C(=O)NC)nc3C)CC2)ncn1] | 5-(4-((6-((N- ethylsulfamoyl)amino)pyrimidin-4- yl)methyl)piperazin-1-yl)-N,6- dimethylpicolinamide |
| 235 | 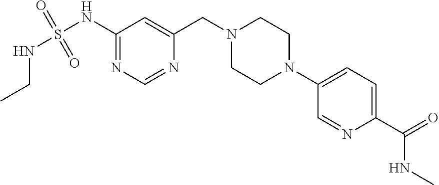 [SMILES: CCNS(=O)(=O)Nc1cc(CN2CCN(c3ccc(C(=O)NC)nc3)CC2)ncn1] | 5-(4-((6-((N- ethylsulfamoyl)amino)pyrimidin-4- yl)methyl)piperazin-1-yl)-N- methylpicolinamide |
| 236 | 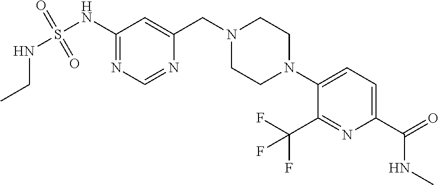 [SMILES: CCNS(=O)(=O)Nc1cc(CN2CCN(c3ccc(C(=O)NC)nc3C(F)(F)F)CC2)ncn1] | 5-(4-((6-((N- etbylsulfamoyl)amino)pyrimidin-4- yl)methyl)piperazin-1-yl)-N-methyl-6- (trifluoromethyl)picolinamide |
| 237 | 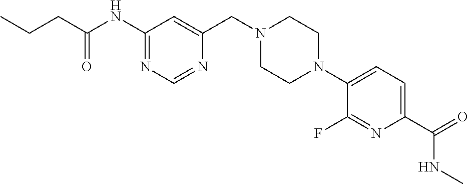 [SMILES: CCCC(=O)Nc1cc(CN2CCN(c3ccc(C(=O)NC)nc3F)CC2)ncn1] | 5-(4-((6-butyramidopyrimidin-4- yl)methyl)piperazin-1-yl)-6-fluoro-N- methylpicolinamide |
| 238 | 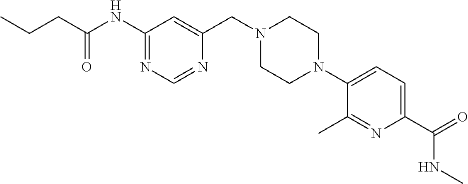 [SMILES: CCCC(=O)Nc1cc(CN2CCN(c3ccc(C(=O)NC)nc3C)CC2)ncn1] | 5-(4-((6-butyramidopyrimidin-4- yl)methyl)piperazin-1-yl)-N,6- dimethylpicolinamide |
| 239 | 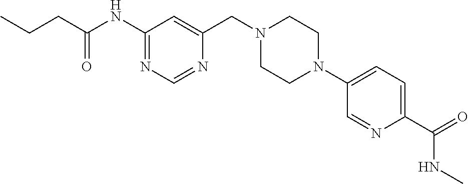 [SMILES: CCCC(=O)Nc1cc(CN2CCN(c3ccc(C(=O)NC)nc3)CC2)ncn1] | 5-(4-((6-butyramidopyrimidin-4- yl)methyl)piperazin-1-yl)-N- methylpicolinamide |
| 240 | 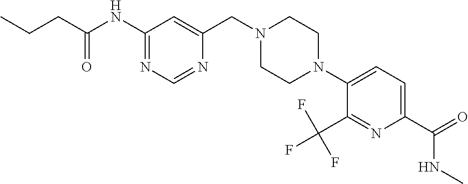 [SMILES: CCCC(=O)Nc1cc(CN2CCN(c3ccc(C(=O)NC)nc3C(F)(F)F)CC2)ncn1] | 5-(4-((6-butyramidopyrimidin-4- yl)methyl)piperazin-1-yl)-N-methyl-6- (trifluoromethyl)picolinamide |
| 241 | 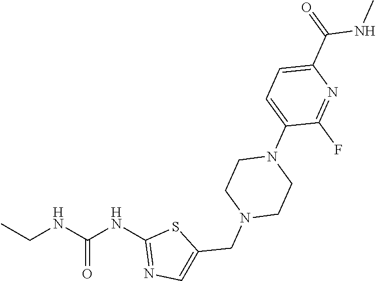 [SMILES: CCNC(=O)Nc1ncc(CN2CCN(c3ccc(C(=O)NC)nc3F)CC2)s1] | 5-(4-((2-(3-ethylureido)thiazol-5- yl)methyl)piperazin-1-yl)-6-fluoro-N- methylpicolinamide |
| 242 | 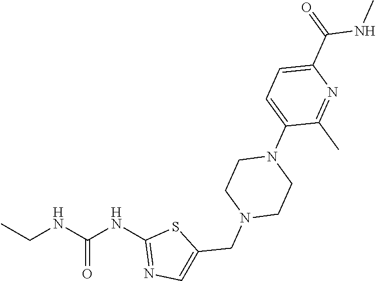 [SMILES: CCNC(=O)Nc1ncc(CN2CCN(c3ccc(C(=O)NC)nc3C)CC2)s1] | 5-(4-((2-(3-ethylureido)thiazol-5- yl)methyl)piperazin-1-yl)-N,6- dimethylpicolinamide |
| 243 | 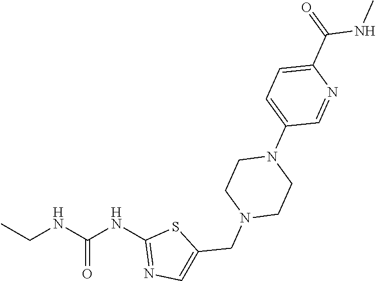 [SMILES: CCNC(=O)Nc1ncc(CN2CCN(c3ccc(C(=O)NC)nc3)CC2)s1] | 5-(4-((2-(3-ethylureido)thiazol-5- yl)methyl)piperazin-1-yl)-N- methylpicolinamide |
| 244 | 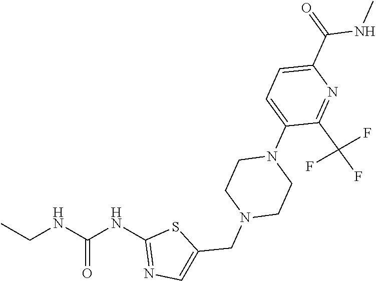 [SMILES: CCNC(=O)Nc1ncc(CN2CCN(c3ccc(C(=O)NC)nc3C(F)(F)F)CC2)s1] | 5-(4-((2-(3-ethylureido)thiazol-5. yl)methyl)piperazin-1-yl)-N-methyl-6- (trifluoromethyl)picolinamide |
| 245 | 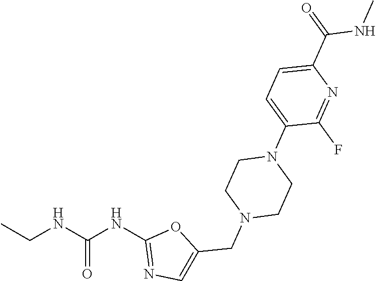 [SMILES: CCNC(=O)Nc1ncc(CN2CCN(c3ccc(C(=O)NC)nc3F)CC2)o1] | 5-(4-((2-(3-ethylureido)oxazol-5- yl)methyl)piperazin-1-yl)-6-fluoro-N- methylpicolinamide |
| 246 | 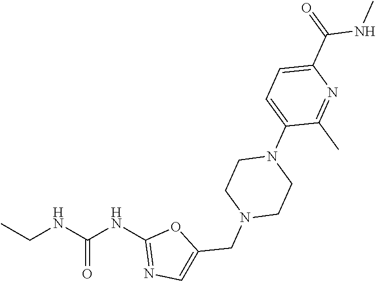 [SMILES: CCNC(=O)Nc1ncc(CN2CCN(c3ccc(C(=O)NC)nc3C)CC2)o1] | 5-(4-((2-(3-ethylureido)oxazol-5- yl)methyl)piperazin-1-yl)-N,6- dimethylpicolinamide |
| 247 | 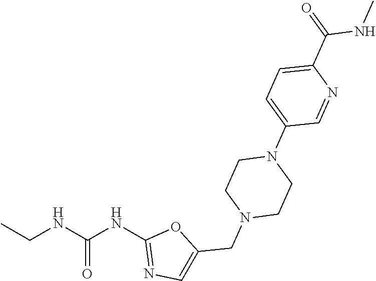 [SMILES: CCNC(=O)Nc1ncc(CN2CCN(c3ccc(C(=O)NC)nc3)CC2)o1] | 5-(4-((2-(3-ethylureido)oxazol-5- yl)methyl)piperazin-1-yl)-N- methylpicolinamide |
| 248 | 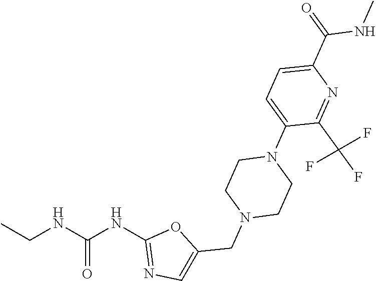 [SMILES: CCNC(=O)Nc1ncc(CN2CCN(c3ccc(C(=O)NC)nc3C(F)(F)F)CC2)o1] | 5-(4-((2-(3-ethylureido)oxazol-5- yl)methyl)piperazin-1-yl)-N-methyl-6- (trifluoromethyl)picolinamide |
| 249 | 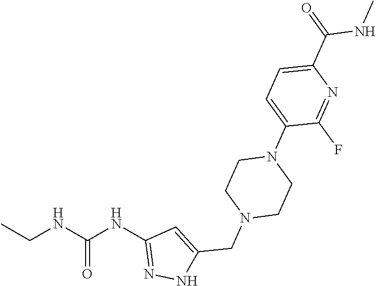 [SMILES: CCNC(=O)Nc1cc(CN2CCN(c3ccc(C(=O)NC)nc3F)CC2)[nH]n1] | 5-(4-((3-(3-ethylureido)-1H-pyrazol-5- yl)methyl)piperazin-1-yl)-6-fluoro-N- methylpicolinamide |
| 250 | 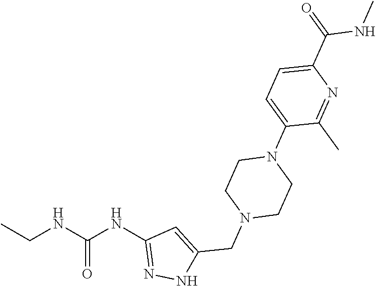 [SMILES: CCNC(=O)Nc1cc(CN2CCN(c3ccc(C(=O)NC)nc3C)CC2)[nH]n1] | 5-(4-((3-(3-ethylureido)-1H-pyrazol-5- yl)methyl)piperazin-1-yl)-N,6- dimethylpicolinamide |
| 251 | 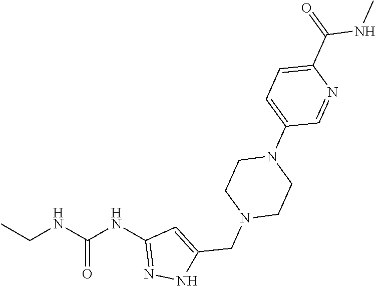 [SMILES: CCNC(=O)Nc1cc(CN2CCN(c3ccc(C(=O)NC)nc3)CC2)[nH]n1] | 5-(4-((3-(3-ethylureido)-1H-pyrazol-5- yl)methyl)piperazin-1-yl)-N- methylpicolinamide |
| 252 | 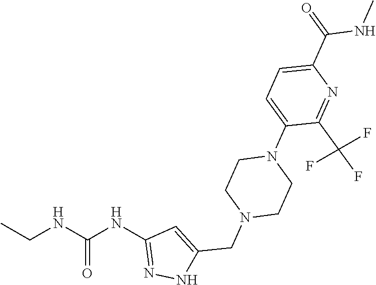 [SMILES: CCNC(=O)Nc1cc(CN2CCN(c3ccc(C(=O)NC)nc3C(F)(F)F)CC2)[nH]n1] | 5-(4-((3-(3-ethylureido)-1H-pyrazol-5- yl)methyl)piperazin-1-yl)-N-methyl-6- (trifluoromethyl)picolinamide |
| 253 | 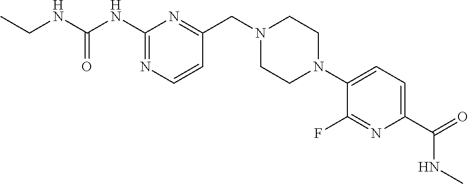 [SMILES: CCNC(=O)Nc1nccc(CN2CCN(c3ccc(C(=O)NC)nc3F)CC2)n1] | 5-(4-((2-(3-ethylureido)pyrimidin-4- yl)methyl)piperazin-1-yl)-6-fluoro-N- methylpicolinamide |
| 254 | 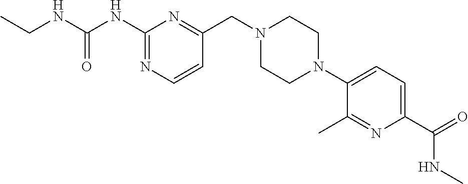 [SMILES: CCNC(=O)Nc1nccc(CN2CCN(c3ccc(C(=O)NC)nc3C)CC2)n1] | 5-(4-((2-(3-ethylureido)pyrimidin-4- yl)methyl)piperazin-1-yl)-N,6- dimethylpicolinamide |
| 255 | 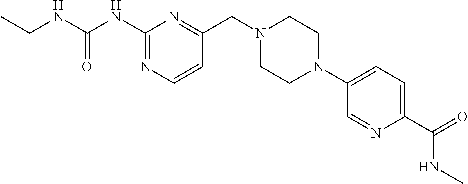 [SMILES: CCNC(=O)Nc1nccc(CN2CCN(c3ccc(C(=O)NC)nc3)CC2)n1] | 5-(4-((2-(3-ethylureido)pyrimidin-4- yl)methyl)piperazin-1-yl)-N- methylpicolinamide |
| 256 | 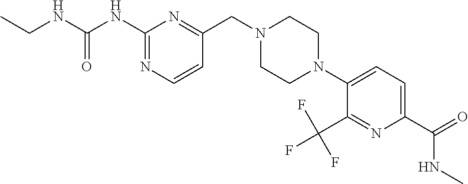 [SMILES: CCNC(=O)Nc1nccc(CN2CCN(c3ccc(C(=O)NC)nc3C(F)(F)F)CC2)n1] | 5-(4-((2-(3-ethylureido)pyrimidin-4- yl)methyl)piperazin-1-yl)-N-methyl-6- (trifluoromethyl)picolinamide |
| 257 | 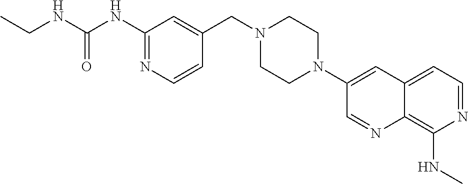 [SMILES: CCNC(=O)Nc1cc(CN2CCN(c3cnc4c(NC)nccc4c3)CC2)ccn1] | 1-ethyl-3-(4-((4-(8-(methylamino)-1,7- naphthyridin-3-yl)piperazin-1- yl)methyl)pyridin-2-yl)urea |
| 258 | 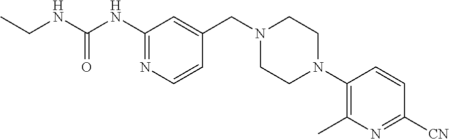 [SMILES: CCNC(=O)Nc1cc(CN2CCN(c3ccc(C#N)nc3C)CC2)ccn1] | 1-(4-((4-(6-cyano-2-methylpyridin-3- yl)piperazin-1-yl)methyl)pyridin-2-yl)-3- ethylurea |
| 259 | 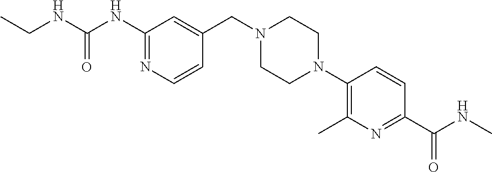 [SMILES: CCNC(=O)Nc1cc(CN2CCN(c3ccc(C(=O)NC)nc3C)CC2)ccn1] | S-(4-((2-(3-isopropylureido)pyridin-4- yl)methyl)piperazin-1-yl)-N,6- dimethylpicolinamide |
| 260 | 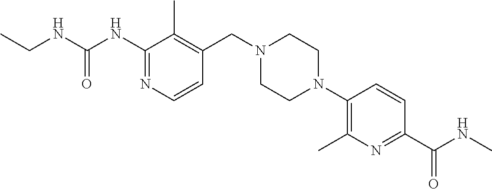 [SMILES: CCNC(=O)Nc1nccc(CN2CCN(c3ccc(C(=O)NC)nc3C)CC2)c1C] | 5-(4-((2-(3-ethylureido)-3-methylpyridin- 4-yl)methyl)piperazin-1-yl)-N,6- dimethylpicolinamide |
| 261 | 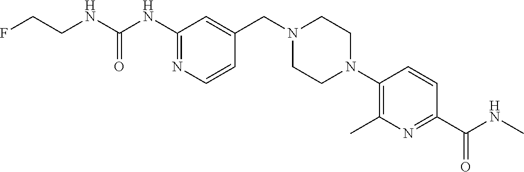 [SMILES: CNC(=O)c1ccc(N2CCN(Cc3ccnc(NC(=O)NCCF)c3)CC2)c(C)n1] | 5-(4-((2-(3-(2-fluoroethyl)ureido)pyridin- 4-yl)methyl)piperazin-1-yl)-N,6- dimethylpicolinamide |
| 262 | 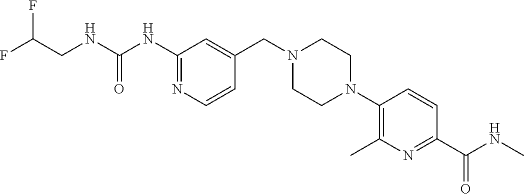 [SMILES: CNC(=O)c1ccc(N2CCN(Cc3ccnc(NC(=O)NCC(F)F)c3)CC2)c(C)n1] | 5-(4-((2-(3-(2,2- difluoroethyl)ureido)pyridin-4- yl)methyl)piperazin-1-yl)-N,6- dimethylpicolinamide |
| 263 | 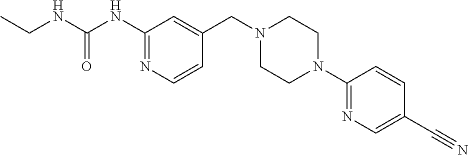 [SMILES: CCNC(=O)Nc1cc(CN2CCN(c3ccc(C#N)cn3)CC2)ccn1] | 1-(4-((4-(5-cyanopyridin-2-yl)piperazin- 1-yl)methyl)pyridin-2-yl)-3-ethylurea |
| 264 | 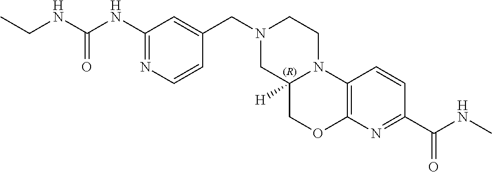 | (R)-3-((2-(3-ethylureido)pyridin-4- yl)methyl)-N-methyl-1,2,3,4,4a,5- hexahydropyrazino[1,2-d]pyrido[2,3- b][1,4]oxazine-8-carboxamide |
| 265 | 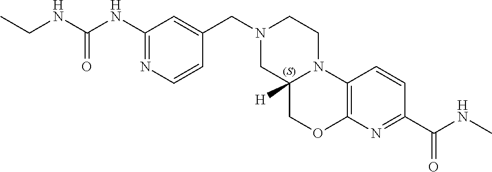 | (S)-3-((2-(3-ethylureido)pyridin-4- yl)methyl)-N-methyl-1,2,3,4,4a,5- hexahydropyrazino[1,2-d]pyrido[2,3- b][1,4]oxazine-8-carboxamide |
| 266 | 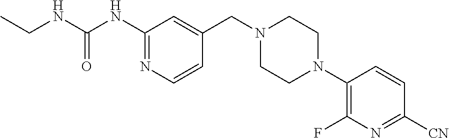 [SMILES: CCNC(=O)Nc1cc(CN2CCN(c3ccc(C#N)nc3F)CC2)ccn1] | 1-(4-((4-(6-cyano-2-fluoropyridin-3- yl)piperazin-1-yl)methyl)pyridin-2-yl)-3- ethylurea |
| 267 | 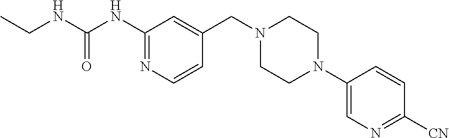 [SMILES: CCNC(=O)Nc1cc(CN2CCN(c3ccc(C#N)nc3)CC2)ccn1] | 1-(4-((4-(6-cyanopyridin-3-yl)piperazin- 1-yl)methyl)pyridin-2-yl)-3-ethylurea |
| 268 | 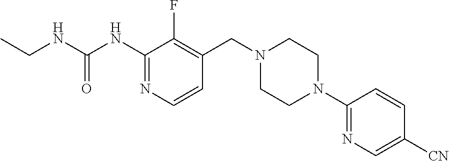 [SMILES: CCNC(=O)Nc1nccc(CN2CCN(c3ccc(C#N)cn3)CC2)c1F] | 1-(4-((4-(5-cyanopyridin-2-yl)piperazin- 1-y)methyl)-3-fluoropyridin-2-yl)-3- ethylurea |
| 269 | 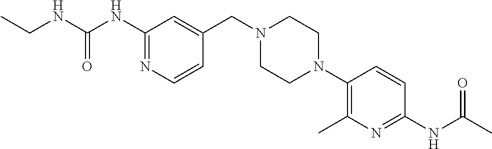 [SMILES: CCNC(=O)Nc1cc(CN2CCN(c3ccc(NC(C)=O)nc3C)CC2)ccn1] | N-(5-(4-((2-(3-ethylureido)pyridin-4- yl)methyl)piperazin-1-yl)-6- methylpyridin-2-yl)acetamide |
| 270 | 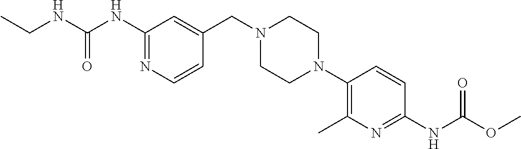 [SMILES: CCNC(=O)Nc1cc(CN2CCN(c3ccc(NC(=O)OC)nc3C)CC2)ccn1] | methyl (5-(4-((2-(3-ethylureido)pyridin-4- yl)methyl)piperazin-1-yl)-6- methylpyridin-2-yl)carbamate |
| 271 | 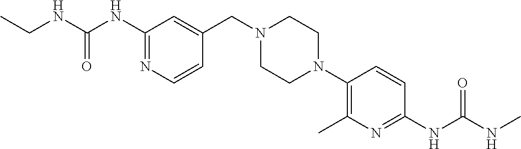 [SMILES: CCNC(=O)Nc1cc(CN2CCN(c3ccc(NC(=O)NC)nc3C)CC2)ccn1] | 1-(5-(4-((2-(3-ethylureido)pyridin-4- yl)methyl)piperazin-1-yl)-6- methylpyridin-2-yl)-3-methylurea |
| 272 | 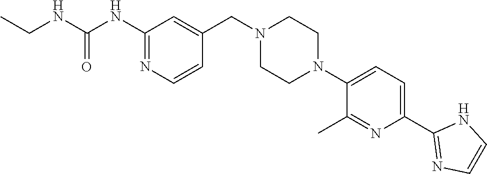 [SMILES: CCNC(=O)Nc1cc(CN2CCN(c3ccc(-c4ncc[nH]4)nc3C)CC2)ccn1] | 1-(4-((4-(6-(1H-imidazol-2-yl)-2- methylpyridin-3-yl)piperazin-1- yl)methyl)pyridin-2-yl)-3-ethylurea |
| 273 | 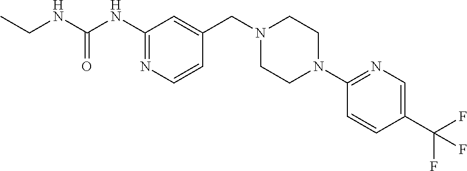 [SMILES: CCNC(=O)Nc1cc(CN2CCN(c3ccc(C(F)(F)F)cn3)CC2)ccn1] | 1-ethyl-3-(4-((4-(5- (trifluoromethyl)pyridin-2-yl)piperazin-1- yl)methyl)pyridin-2-yl)urea |
| 274 | 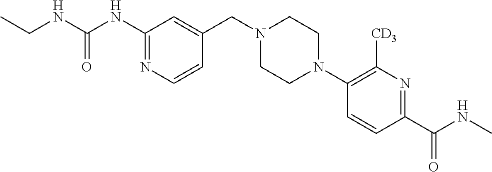 [SMILES: [2H]C([2H])([2H])c1nc(C(=O)NC)ccc1N1CCN(Cc2ccnc(NC(=O)NCC)c2)CC1] | 5-(4-((2-(3-ethylureido) pyridin-4-yl) methyl) piperazin-1-yl)-N-methyl-6- (methyl-d3) picolinamide |
| 275 | 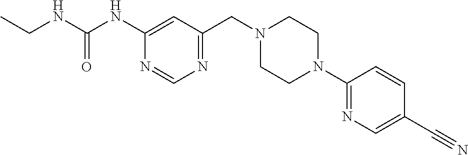 [SMILES: CCNC(=O)Nc1cc(CN2CCN(c3ccc(C#N)cn3)CC2)ncn1] | 1-(6-((4-(5-cyanopyridin-2-yl)piperazin- 1-yl)methyl)pyrimidin-4-yl)-3-ethylurea |
| 276 | 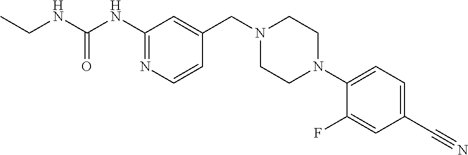 [SMILES: CCNC(=O)Nc1cc(CN2CCN(c3ccc(C#N)cc3F)CC2)ccn1] | 1-(4-((4-(4-cyano-2- fluorophenyl)piperazin-1- yl)methyl)pyridin-2-yl)-3-ethylurea |
| 277 | 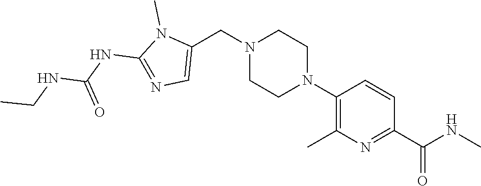 [SMILES: CCNC(=O)Nc1ncc(CN2CCN(c3ccc(C(=O)NC)nc3C)CC2)n1C] | 5-(4-((2-(3-ethylureido)-1-methyl-1H- imidazol-5-yl)methyl)piperazin-1-yl)- N,6-dimethylpicolinamide |
| 278 | 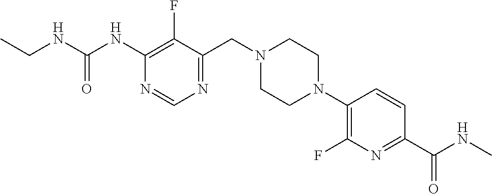 [SMILES: CCNC(=O)Nc1ncnc(CN2CCN(c3ccc(C(=O)NC)nc3F)CC2)c1F] | 5-(4-((6-(3-ethylureido)-5- fluoropyrimidin-4-yl)methyl)piperazin-1- yl)-6-fluoro-N-methylpicolinamide |
| 279 | 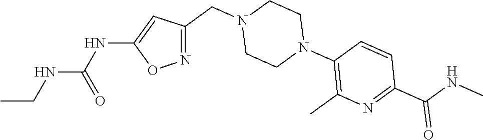 [SMILES: CCNC(=O)Nc1cc(CN2CCN(c3ccc(C(=O)NC)nc3C)CC2)no1] | 5-(4-((5-(3-ethylureido)isoxazol-3- yl)methyl)piperazin-1-yl)-N,6- dimethylpicolinamide |
| 280 | 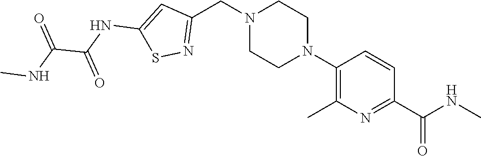 | N1-methyl-N2-(3-((4-(2-methyl-6- (methylcarbamoyl)pyridin-3-yl)piperazin- 1-yl)methyl)isothiazol-5-yl)oxalamide |
| 281 | 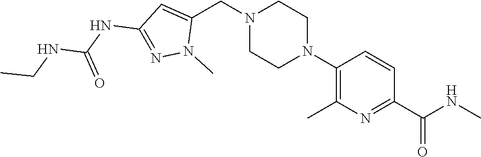 [SMILES: CCNC(=O)Nc1cc(CN2CCN(c3ccc(C(=O)NC)nc3C)CC2)n(C)n1] | 5-(4-((3-(3-ethylureido)-1-methyl-1H- pyrazol-5-yl)methyl)piperazin-1-yl)-N,6- dimethylpicolinamide |
| 282 | 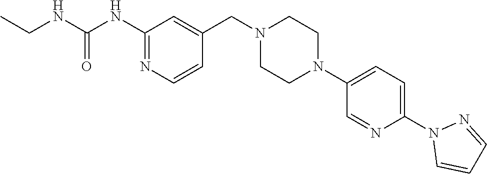 [SMILES: CCNC(=O)Nc1cc(CN2CCN(c3ccc(-n4cccn4)nc3)CC2)ccn1] | 1-(4-((4-(6-(1H-pyrazol-1-yl)pyridin-3- yl)piperazin-1-yl)methyl)pyridin-2-yl)-3- ethylurea |
| 283 | 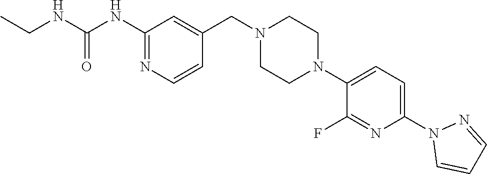 [SMILES: CCNC(=O)Nc1cc(CN2CCN(c3ccc(-n4cccn4)nc3F)CC2)ccn1] | 1-ethyl-3-(4-((4-(2-fluoro-6-(1H-pyrazol- 1-yl)pyridin-3-yl)piperazin-1- yl)methyl)pyridin-2-yl)urea |
| 284 | 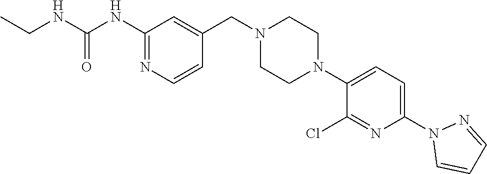 [SMILES: CCNC(=O)Nc1cc(CN2CCN(c3ccc(-n4cccn4)nc3Cl)CC2)ccn1] | 1-(4-((4-(2-chloro-6-(1H-pyrazol-1- yl)pyridin-3-yl)piperazin-1- yl)methyl)pyridin-2-yl)-3-ethylurea |
| 285 | 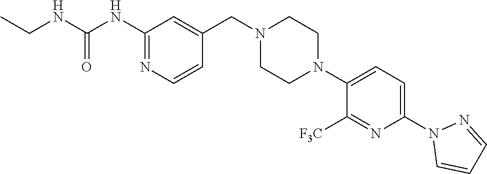 [SMILES: CCNC(=O)Nc1cc(CN2CCN(c3ccc(-n4cccn4)nc3C(F)(F)F)CC2)ccn1] | 1-(4-((4-(6-(1H-pyrazol-1-yl)-2- (trifluoromethyl)pyridin-3-yl)piperazin-1- yl)methyl)pyridin-2-yl)-3-ethylurea |
| 286 | 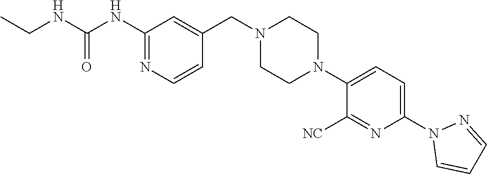 [SMILES: CCNC(=O)Nc1cc(CN2CCN(c3ccc(-n4cccn4)nc3C#N)CC2)ccn1] | 1-(4-((4-(2-cyano-6-(1H-pyrazol-1- yl)pyridin-3-yl)piperazin-1- yl)methyl)pyridin-2-yl)-3-ethylurea |
| 287 | 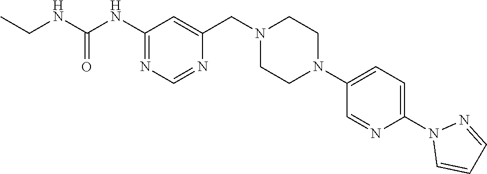 [SMILES: CCNC(=O)Nc1cc(CN2CCN(c3ccc(-n4cccn4)nc3)CC2)ncn1] | 1-(6-((4-(6-(1H-pyrazol-1-yl)pyridin-3- yl)piperazin-1-yl)methyl)pyrimidin-4-yl)- 3-ethylurea |
| 288 | 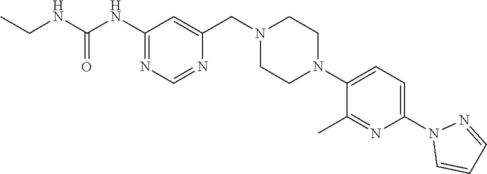 [SMILES: CCNC(=O)Nc1cc(CN2CCN(c3ccc(-n4cccn4)nc3C)CC2)ncn1] | 1-ethyl-3-(6-((4-(2-methyl-6-(1H- pyrazol-1-yl)pyridin-3-yl)piperazin-1- yl)methyl)pyrimidin-4-yl)urea |
| 289 | 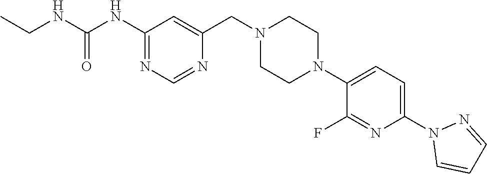 [SMILES: CCNC(=O)Nc1cc(CN2CCN(c3ccc(-n4cccn4)nc3F)CC2)ncn1] | 1-ethyl-3-(6-((4-(2-fluoro-6-(1H-pyrazol- 1-yl)pyridin-3-yl)piperazin-1- yl)methyl)pyrimidin-4-yl)urea |
| 290 | 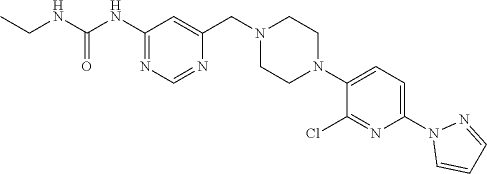 [SMILES: CCNC(=O)Nc1cc(CN2CCN(c3ccc(-n4cccn4)nc3Cl)CC2)ncn1] | 1-(6-((4-(2-chloro-6-(1H-pyrazol-1- yl)pyridin-3-yl)piperazin-1- yl)methyl)pyrimidin-4-yl)-3-ethylurea |
| 291 | 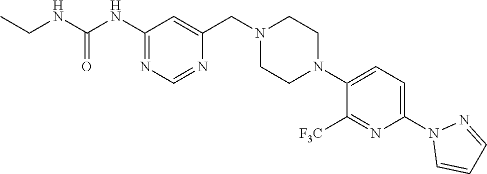 [SMILES: CCNC(=O)Nc1cc(CN2CCN(c3ccc(-n4cccn4)nc3C(F)(F)F)CC2)ncn1] | 1-(6-((4-(6-(1H-pyrazol-1-yl)-2- (trifluoromethyl)pyridin-3-yl)piperazin-1- yl)methyl)pyrimidin-4-yl)-3-ethylurea |
| 292 | 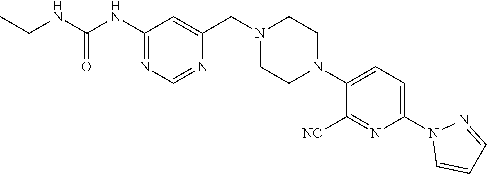 [SMILES: CCNC(=O)Nc1cc(CN2CCN(c3ccc(-n4cccn4)nc3C#N)CC2)ncn1] | 1-(6-((4-(2-cyano-6-(1H-pyrazol-1- yl)pyridin-3-yl)piperazin-1- yl)methyl)pyrimidin-4-yl)-3-ethylurea |
| 293 | 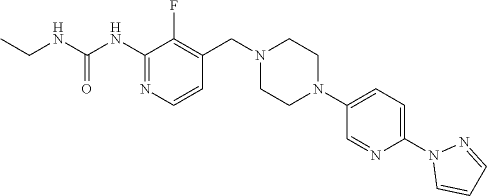 [SMILES: CCNC(=O)Nc1nccc(CN2CCN(c3ccc(-n4cccn4)nc3)CC2)c1F] | 1-(4-((4-(6-(1H-pyrazol-1-yl)pyridin-3- yl)piperazin-1-yl)methyl)-3- fluoropyridin-2-yl)-3-ethylurea |
| 294 | 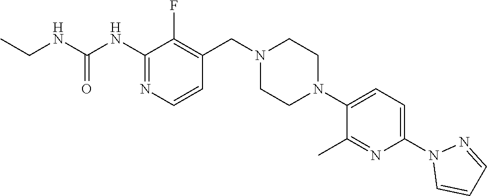 [SMILES: CCNC(=O)Nc1nccc(CN2CCN(c3ccc(-n4cccn4)nc3C)CC2)c1F] | 1-ethyl-3-(3-fluoro-4-((4-(2-methyl-6- (1H-pyrazol-1-yl)pyridin-3-yl)piperazin- 1-yl)methyl)pyridin-2-yl)urea |
| 295 | 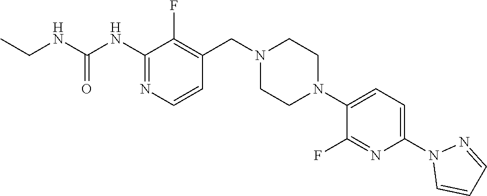 [SMILES: CCNC(=O)Nc1nccc(CN2CCN(c3ccc(-n4cccn4)nc3F)CC2)c1F] | 1-ethyl-3-(3-fluoro-4-((4-(2-fluoro-6-(1H- pyrazol-1-yl)pyridin-3-yl)piperazin-1- yl)methyl)pyridin-2-yl)urea |
| 296 | 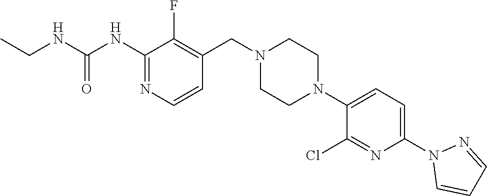 [SMILES: CCNC(=O)Nc1nccc(CN2CCN(c3ccc(-n4cccn4)nc3Cl)CC2)c1F] | 1-(4-((4-(2-chloro-6-(1H-pyrazol-1- yl)pyridin-3-yl)piperazin-1-yl)methyl)-3- fluoropyridin-2-yl)-3-ethylurea |
| 297 | 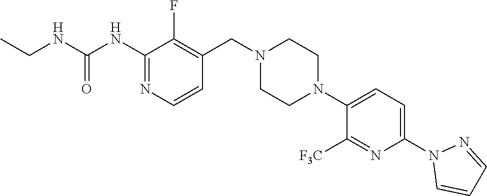 [SMILES: CCNC(=O)Nc1nccc(CN2CCN(c3ccc(-n4cccn4)nc3C(F)(F)F)CC2)c1F] | 1-(4-((4-(6-(1H-pyrazol-1-yl)-2- (trifluoromethyl)pyridin-3-yl)piperazin-1- yl)methyl)-3-fluoropyridin-2-yl)-3- ethylurea |
| 298 | 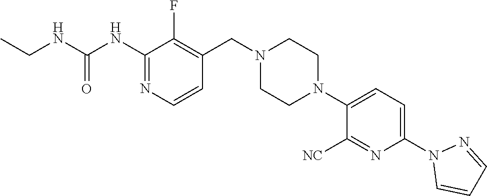 [SMILES: CCNC(=O)Nc1nccc(CN2CCN(c3ccc(-n4cccn4)nc3C#N)CC2)c1F] | 1-(4-((4-(2-cyano-6-(1H-pyrazol-1- yl)pyridin-3-yl)piperazin-1-yl)methyl)-3- fluoropyridin-2-yl)-3-ethylurea |
| 299 | 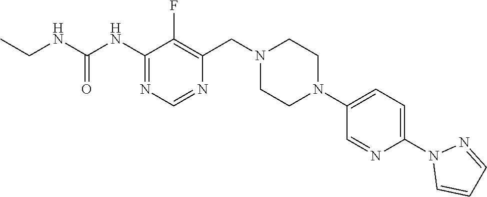 [SMILES: CCNC(=O)Nc1ncnc(CN2CCN(c3ccc(-n4cccn4)nc3)CC2)c1F] | 1-(6-((4-(6-(1H-pyrazol-1-yl)pyridin-3- yl)piperazin-1-yl)methyl)-5- fluoropyrimidin-4-yl)-3-ethylurea |
| 300 | 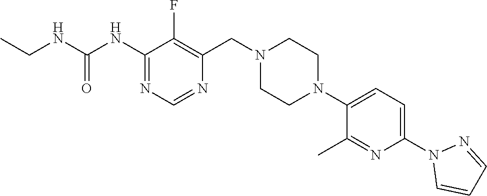 [SMILES: CCNC(=O)Nc1ncnc(CN2CCN(c3ccc(-n4cccn4)nc3C)CC2)c1F] | 1-ethyl-3-(5-fluoro-6-((4-(2-methyl-6- (1H-pyrazol-1-yl)pyridin-3-yl)piperazin- 1-yl)methyl)pyrimidin-4-yl)urea |
| 301 | 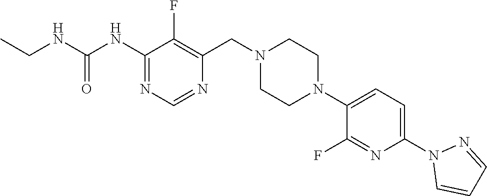 [SMILES: CCNC(=O)Nc1ncnc(CN2CCN(c3ccc(-n4cccn4)nc3F)CC2)c1F] | 1-ethyl-3-(5-fluoro-6-((4-(2-fluoro-6-(1H- pyrazol-1-yl)pyridin-3-yl)piperazin-1- yl)methyl)pyrimidin-4-yl)urea |
| 302 | 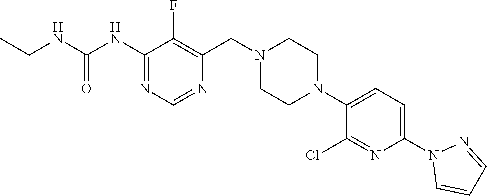 [SMILES: CCNC(=O)Nc1ncnc(CN2CCN(c3ccc(-n4cccn4)nc3Cl)CC2)c1F] | 1-(6-((4-(2-chloro-6-(1H-pyrazol-1- yl)pyridin-3-yl)piperazin-1-yl)methyl)-5- fluoropyrimidin-4-yl)-3-ethylurea |
| 303 | 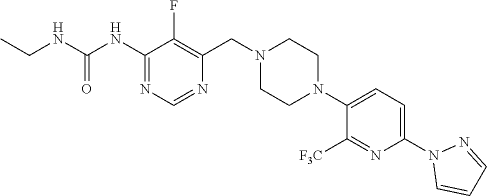 [SMILES: CCNC(=O)Nc1ncnc(CN2CCN(c3ccc(-n4cccn4)nc3C(F)(F)F)CC2)c1F] | 1-(6-((4-(6-(1H-pyrazol-1-yl)-2- (trifluoromethyl)pyridin-3-yl)piperazin-1- yl)methyl)-5-fluoropyrimidin-4-yl)-3- ethylurea |
| 304 | 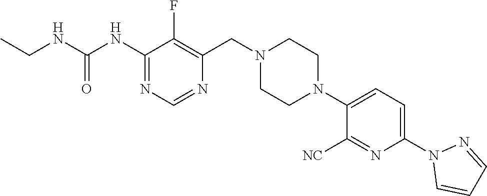 [SMILES: CCNC(=O)Nc1ncnc(CN2CCN(c3ccc(-n4cccn4)nc3C#N)CC2)c1F] | 1-(6-((4-(2-cyano-6-(1H-pyrazol-1- yl)pyridin-3-yl)piperazin-1-yl)methyl)-5- fluoropyrimidin-4-yl)-3-ethylurea |
| 305 | 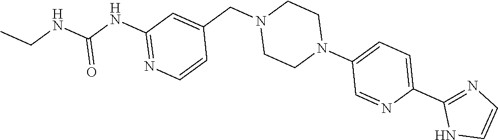 [SMILES: CCNC(=O)Nc1cc(CN2CCN(c3ccc(-c4ncc[nH]4)nc3)CC2)ccn1] | 1-(4-((4-(6-(1H-imidazol-2-yl)pyridin-3- yl)piperazin-1-yl)methyl)pyridin-2-yl)-3- ethylurea |
| 306 | 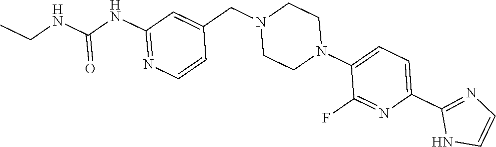 [SMILES: CCNC(=O)Nc1cc(CN2CCN(c3ccc(-c4ncc[nH]4)nc3F)CC2)ccn1] | 1-ethyl-3-(4-((4-(2-fluoro-6-(1H- imidazol-2-yl)pyridin-3-yl)piperazin-1- yl)methyl)pyridin-2-yl)urea |
| 307 | 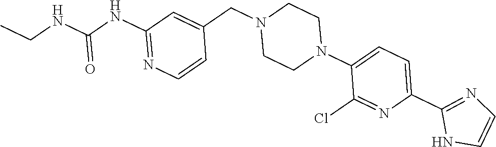 [SMILES: CCNC(=O)Nc1cc(CN2CCN(c3ccc(-c4ncc[nH]4)nc3Cl)CC2)ccn1] | 1-(4-((4-(2-chloro-6-(1H-imidazol-2- yl)pyridin-3-yl)piperazin-1- yl)methyl)pyridin-2-yl)-3-ethylurea |
| 308 | 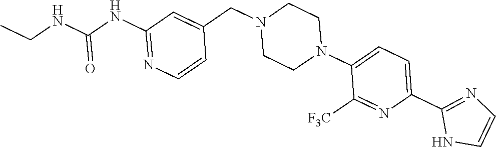 [SMILES: CCNC(=O)Nc1cc(CN2CCN(c3ccc(-c4ncc[nH]4)nc3C(F)(F)F)CC2)ccn1] | 1-(4-((4-(6-(1H-imidazol-2-yl)-2- (trifluoromethyl)pyridin-3-yl)piperazin-1- yl)methyl)pyridin-2-yl)-3-ethylurea |
| 309 | 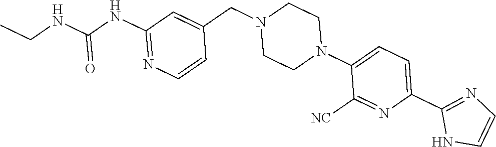 [SMILES: CCNC(=O)Nc1cc(CN2CCN(c3ccc(-c4ncc[nH]4)nc3C#N)CC2)ccn1] | 1-(4-((4-(2-cyano-6-(1H-imidazol-2- yl)pyridin-3-yl)piperazin-1- yl)methyl)pyridin-2-yl)-3-ethylurea |
| 310 | 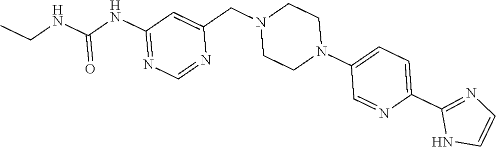 [SMILES: CCNC(=O)Nc1cc(CN2CCN(c3ccc(-c4ncc[nH]4)nc3)CC2)ncn1] | 1-(6-((4-(6-(1H-imidazol-2-yl)pyridin-3- yl)piperazin-1-yl)methyl)pyrimidin-4-yl)- 3-ethylurea |
| 311 | 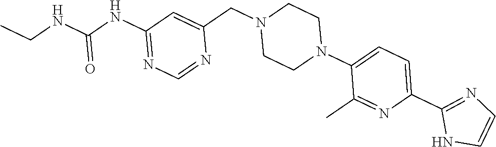 [SMILES: CCNC(=O)Nc1cc(CN2CCN(c3ccc(-c4ncc[nH]4)nc3C)CC2)ncn1] | 1-(6-((4-(6-(1H-imidazol-2-yl)-2- methylpyridin-3-yl)piperazin-1- yl)methyl)pyrimidin-4-yl)-3-ethylurea |
| 312 | 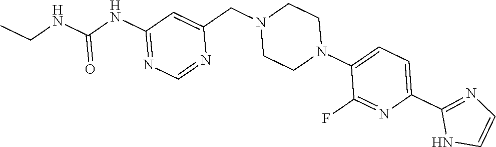 [SMILES: CCNC(=O)Nc1cc(CN2CCN(c3ccc(-c4ncc[nH]4)nc3F)CC2)ncn1] | 1-ethyl-3-(6-((4-(2-fluoro-6-(1H- imidazol-2-yl)pyridin-3-yl)piperazin-1- yl)methyl)pyrimidin-4-yl)urea |
| 313 | 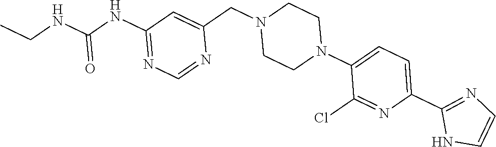 [SMILES: CCNC(=O)Nc1cc(CN2CCN(c3ccc(-c4ncc[nH]4)nc3Cl)CC2)ncn1] | 1-(6-((4-(2-chloro-6-(1H-imidazol-2- yl)pyridin-3-yl)piperazin-1- yl)methyl)pyrimidin-4-yl)-3-ethylurea |
| 314 | 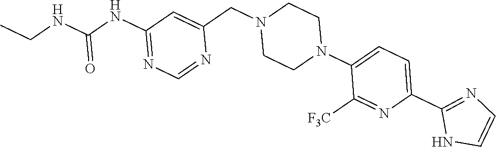 [SMILES: CCNC(=O)Nc1cc(CN2CCN(c3ccc(-c4ncc[nH]4)nc3C(F)(F)F)CC2)ncn1] | 1-(6-((4-(6-(1H-imidazol-2-yl)-2- (trifluoromethyl)pyridin-3-yl)piperazin-1- yl)methyl)pyrimidin-4-yl)-3-ethylurea |
| 315 | 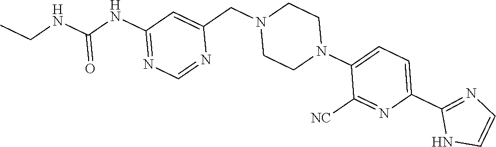 [SMILES: CCNC(=O)Nc1cc(CN2CCN(c3ccc(-c4ncc[nH]4)nc3C#N)CC2)ncn1] | 1-(6-((4-(2-cyano-6-(1H-imidazol-2- yl)pyridin-3-yl)piperazin-1- yl)methyl)pyrimidin-4-yl)-3-ethylurea |
| 316 | 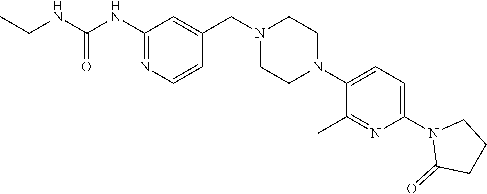 [SMILES: CCNC(=O)Nc1cc(CN2CCN(c3ccc(N4CCCC4=O)nc3C)CC2)ccn1] | 1-ethyl-3-(4-((4-(2-methyl-6-(2- oxopyrrolidin-1-yl)pyridin-3- yl)piperazin-1-yl)methyl)pyridin-2- yl)urca |
| 317 | 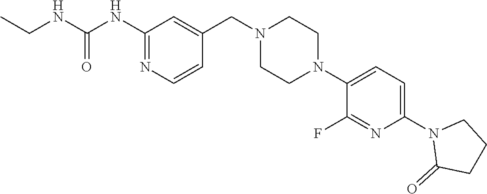 [SMILES: CCNC(=O)Nc1cc(CN2CCN(c3ccc(N4CCCC4=O)nc3F)CC2)ccn1] | 1-ethyl-3-(4-((4-(2-fluoro-6-(2- oxopyrrolidin-1-yl)pyridin-3- yl)piperazin-1-yl)methyl)pyridin-2- yI)urea |
| 318 | 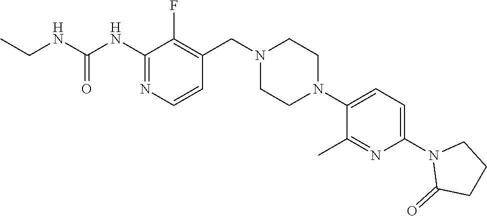 [SMILES: CCNC(=O)Nc1nccc(CN2CCN(c3ccc(N4CCCC4=O)nc3C)CC2)c1F] | 1-ethyl-3-(3-fluoro-4-((4-(2-methyl-6-(2- oxopyrrolidin-1-yl)pyridin-3- yl)piperazin-1-yl)methyl)pyridin-2- yl)urea |
| 319 | 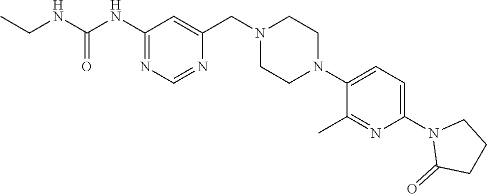 [SMILES: CCNC(=O)Nc1cc(CN2CCN(c3ccc(N4CCCC4=O)nc3C)CC2)ncn1] | 1-ethyl-3-(6-((4-(2-methyl-6-(2- oxopyrrolidin-1-yl)pyridin-3- yl)piperazin-1-yl)methyl)pyrimidin-4- yl)urea |
| 320 | 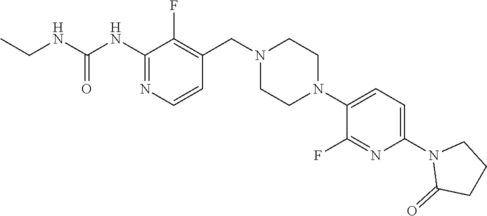 [SMILES: CCNC(=O)Nc1nccc(CN2CCN(c3ccc(N4CCCC4=O)nc3F)CC2)c1F] | 1-ethyl-3-(3-fluoro-4-((4-(2-fluoro-6-(2- oxopyrrolidin-1-yl)pyridin-3- yl)piperazin-1-yl)methyl)pyridin-2- ylurea |
| 321 | 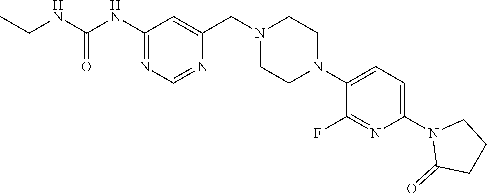 [SMILES: CCNC(=O)Nc1cc(CN2CCN(c3ccc(N4CCCC4=O)nc3F)CC2)ncn1] | 1-ethyl-3-(6-((4-(2-fluoro-6-(2- oxopyrrolidin-1-yl)pyridin-3- yl)piperazin-1-yl)methyl)pyrimidin-4- yl)urea |
| 322 | 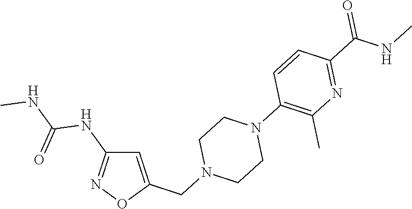 | N,6-dimethyl-5-(4-((3-(3- methylureido)isoxazol-5- yl)methyl)piperazin-1-yl)picolinamide |
| 323 | 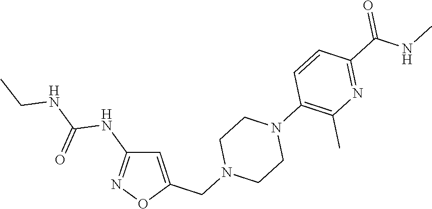 [SMILES: CCNC(=O)Nc1cc(CN2CCN(c3ccc(C(=O)NC)nc3C)CC2)on1] | 5-(4-((3-(3-ethylureido)isoxazol-5. yl)methyl)piperazin-1-yl)-N,6- dimethylpicolinamide |
| 324 | 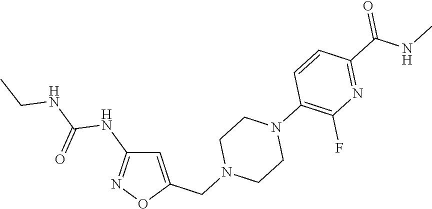 [SMILES: CCNC(=O)Nc1cc(CN2CCN(c3ccc(C(=O)NC)nc3F)CC2)on1] | 5-(4-((3-(3-ethylureido)isoxazol-5- yl)methyl)piperazin-1-yl)-6-fluoro-N- methylpicolinamide |
| 325 | 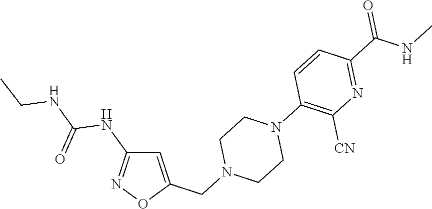 [SMILES: CCNC(=O)Nc1cc(CN2CCN(c3ccc(C(=O)NC)nc3C#N)CC2)on1] | 6-cyano-5-(4-((3-(3-ethylureido)isoxazol- 5-yl)methyl)piperazin-1-yl)-N- methylpicolinamide |
| 326 | 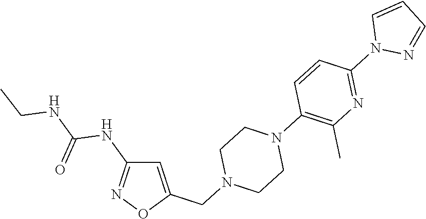 [SMILES: CCNC(=O)Nc1cc(CN2CCN(c3ccc(-n4cccn4)nc3C)CC2)on1] | 1-ethyl-3-(5-((4-(2-methyl-6-(1H- pyrazol-1-yl)pyridin-3-yl)piperazin-1- yl)methyl)isoxazol-3-yl)urea |
| 327 | 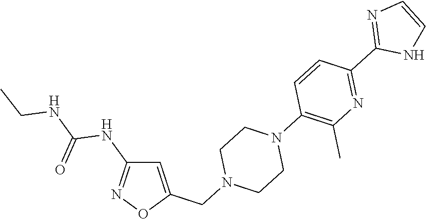 [SMILES: CCNC(=O)Nc1cc(CN2CCN(c3ccc(-c4ncc[nH]4)nc3C)CC2)on1] | 1-(5-((4-(6-(1H-imidazol-2-yl)-2- methylpyridin-3-yl)piperazin-1- yl)methyl)isoxazol-3-yl)-3-ethylurea |
| 328 | 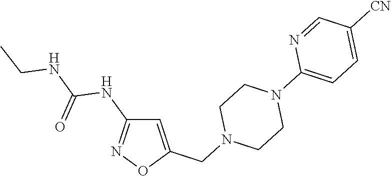 [SMILES: CCNC(=O)Nc1cc(CN2CCN(c3ccc(C#N)cn3)CC2)on1] | 1-(5-((4-(5-cyanopyridin-2-yl)piperazin- 1-yl)methyl)isoxazol-3-yl)-3-ethylurea |
| 329 | 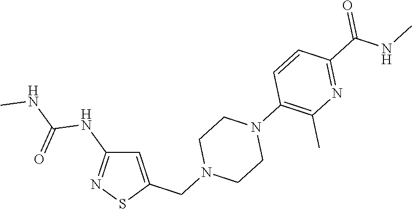 | N,6-dimethyl-5-(4-((3-(3- methylureido)isothiazol-5- yl)methyl)piperazin-1-yl)picolinamide |
| 330 | 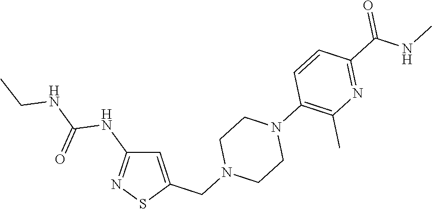 [SMILES: CCNC(=O)Nc1cc(CN2CCN(c3ccc(C(=O)NC)nc3C)CC2)sn1] | 5-(4-((3-(3-ethylureido)isothiazol-5- yl)methyl)piperazin-1-yl)-N,6- dimethylpicolinamide |
| 331 | 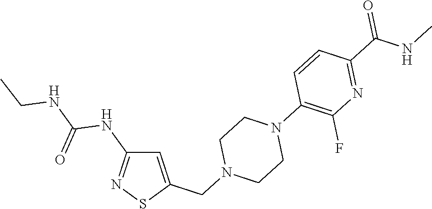 [SMILES: CCNC(=O)Nc1cc(CN2CCN(c3ccc(C(=O)NC)nc3F)CC2)sn1] | 5-(4-((3-(3-ethylureido)isothiazol-5- yl)methyl)piperazin-1-yl)-6-fluoro-N- methylpicolinamide |
| 332 | 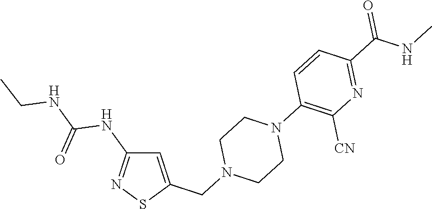 [SMILES: CCNC(=O)Nc1cc(CN2CCN(c3ccc(C(=O)NC)nc3C#N)CC2)sn1] | 6-cyano-5-(4-((3-(3- ethylureido)isothiazol-5- yl)methyl)piperazin-1-yl)-N- methylpicolinamide |
| 333 | 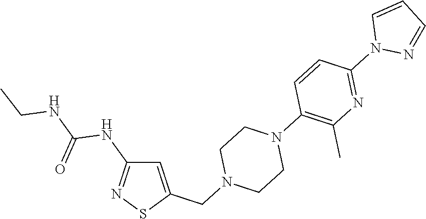 [SMILES: CCNC(=O)Nc1cc(CN2CCN(c3ccc(-n4cccn4)nc3C)CC2)sn1] | 1-ethyl-3-(5-((4-(2-methyl-6-(1H- pyrazol-1-yl)pyridin-3-yl)piperazin-1- yl)methyl)isothiazol-3-yl)urea |
| 334 | 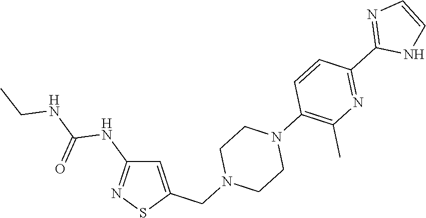 [SMILES: CCNC(=O)Nc1cc(CN2CCN(c3ccc(-c4ncc[nH]4)nc3C)CC2)sn1] | 1-(5-((4-(6-(1H-imidazol-2-yl)-2- methylpyridin-3-yl)piperazin-1- yl)methyl)isothiazol-3-yl)-3-ethylurea |
| 335 | 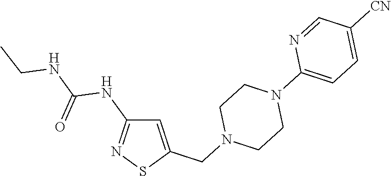 [SMILES: CCNC(=O)Nc1cc(CN2CCN(c3ccc(C#N)cn3)CC2)sn1] | 1-(5-((4-(5-cyanopyridin-2-yl)piperazin- 1-yl)methyl)isothiazol-3-yl)-3-ethylurea |
| 336 | 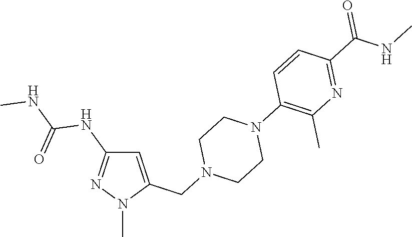 | N,6-dimethyl-5-(4-((1-methyl-3-(3- methylureido)-1H-pyrazol-5- yl)methyl)piperazin-1-yl)picolinamide |
| 337 | 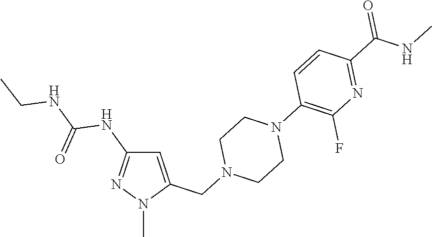 [SMILES: CCNC(=O)Nc1cc(CN2CCN(c3ccc(C(=O)NC)nc3F)CC2)n(C)n1] | 5-(4-((3-(3-ethylureido)-1-methyl-1H- pyrazol-5-yl)methyl)piperazin-1-yl)-6- fluoro-N-methylpicolinamide |
| 338 | 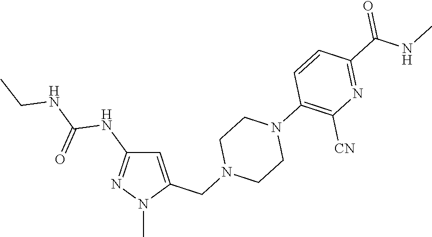 [SMILES: CCNC(=O)Nc1cc(CN2CCN(c3ccc(C(=O)NC)nc3C#N)CC2)n(C)n1] | 6-cyano-5-(4-((3-(3-ethylureido)-1- methyl-1H-pyrazol-5- yl)methyl)piperazin-1-yl)-N- methylpicolinamide |
| 339 | 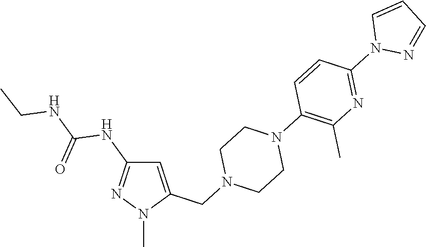 [SMILES: CCNC(=O)Nc1cc(CN2CCN(c3ccc(-n4cccn4)nc3C)CC2)n(C)n1] | 1-ethyl-3-(1-methyl-5-((4-(2-methyl-6- (1H-pyrazol-1-yl)pyridin-3-yl)piperazin- 1-yl)methyl)-1H-pyrazol-3-yl)urea |
| 340 | 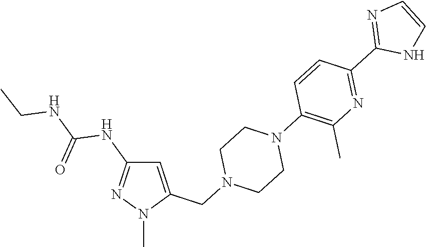 [SMILES: CCNC(=O)Nc1cc(CN2CCN(c3ccc(-c4ncc[nH]4)nc3C)CC2)n(C)n1] | 1-(5-((4-(6-(1H-imidazol-2-yl)-2- methylpyridin-3-yl)piperazin-1- yl)methyl)-1-methyl-1H-pyrazol-3-yl)-3- ethylurea |
| 341 | 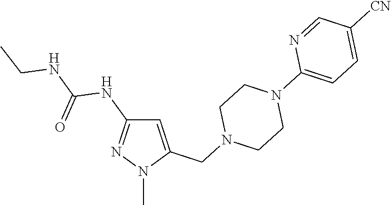 [SMILES: CCNC(=O)Nc1cc(CN2CCN(c3ccc(C#N)cn3)CC2)n(C)n1] | 1-(5-((4-(5-cyanopyridin-2-yl)piperazin- 1-yl)methyl)-1-methyl-1H-pyrazol-3-yl)- 3-ethylurea |
| 342 | 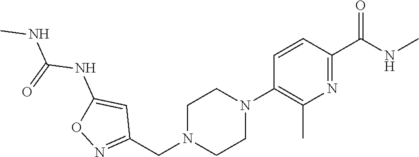 | N,6-dimethyl-5-(4-((5-(3- methylureido)isoxazol-3- yl)methyl)piperazin-1-yl)picolinamide |
| 343 | 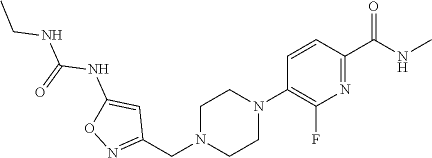 [SMILES: CCNC(=O)Nc1cc(CN2CCN(c3ccc(C(=O)NC)nc3F)CC2)no1] | 5-(4-((5-(3-ethylureido)isoxazol-3- yl)methyl)piperazin-1-yl)-6-fluoro-N- methylpicolinamide |
| 344 | 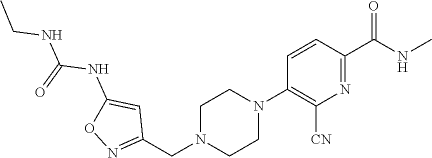 [SMILES: CCNC(=O)Nc1cc(CN2CCN(c3ccc(C(=O)NC)nc3C#N)CC2)no1] | 6-cyano-S-(4-((5-(3-ethylureido)isoxazol- 3-yl)methyl)piperazin-1-yl)-N- methylpicolinamide |
| 345 | 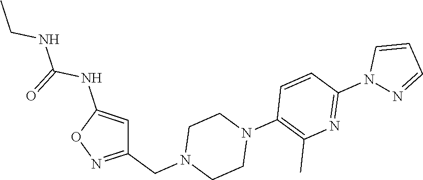 [SMILES: CCNC(=O)Nc1cc(CN2CCN(c3ccc(-n4cccn4)nc3C)CC2)no1] | 1-ethyl-3-(3-((4-(2-methyl-6-(1H- pyrazol-1-yl)pyridin-3-yl)piperazin-1- yl)methyl)isoxazol-5-yl)urea |
| 346 | 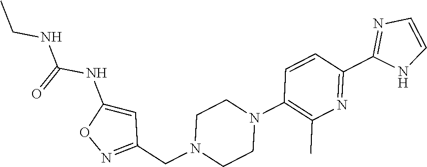 [SMILES: CCNC(=O)Nc1cc(CN2CCN(c3ccc(-c4ncc[nH]4)nc3C)CC2)no1] | 1-(3-((4-(6-(1H-imidazol-2-yl)-2- methylpyridin-3-yl)piperazin-1- yl)methyl)isoxazol-5-yl)-3-ethylurea |
| 347 | 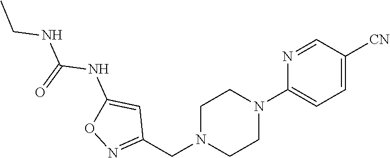 [SMILES: CCNC(=O)Nc1cc(CN2CCN(c3ccc(C#N)cn3)CC2)no1] | 1-(3-((4-(5-cyanopyridin-2-yl)piperazin- 1-yl)methyl)isoxazol-5-yl)-3-ethylurea |
| 348 | 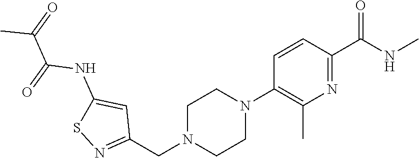 [SMILES: CNC(=O)c1ccc(N2CCN(Cc3cc(NC(=O)C(C)=O)sn3)CC2)c(C)n1] | N,6-dimethyl-5-(4-((5-(2- oxopropanamido)isothiazol-3- yl)methyl)piperazin-1-yl)picolinamide |
| 349 | 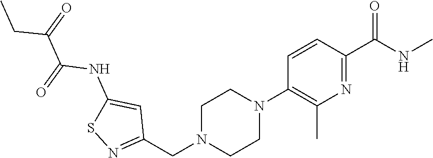 [SMILES: CCC(=O)C(=O)Nc1cc(CN2CCN(c3ccc(C(=O)NC)nc3C)CC2)ns1] | N,6-dimethyl-5-(4-((5-(2- oxobutanamido)isothiazol-3- yl)methyl)piperazin-1-yl)picolinamide |
| 350 | 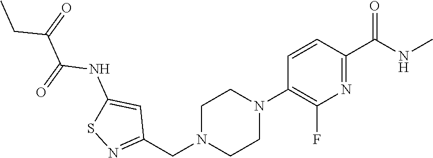 [SMILES: CCC(=O)C(=O)Nc1cc(CN2CCN(c3ccc(C(=O)NC)nc3F)CC2)ns1] | 6-fluoro-N-methyl-5-(4-((5-(2- oxobutanamido)isothiazol-3- yl)methyl)piperazin-1-yl)picolinamide |
| 351 | 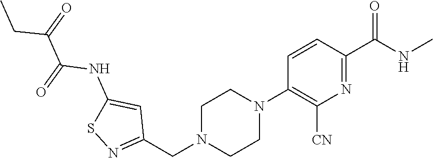 [SMILES: CCC(=O)C(=O)Nc1cc(CN2CCN(c3ccc(C(=O)NC)nc3C#N)CC2)ns1] | 6-cyano-N-methyl-5-(4-((5-(2- oxobutanamido)isothiazol-3- yl)methyl)piperazin-1-yl)picolinamide |
| 352 | 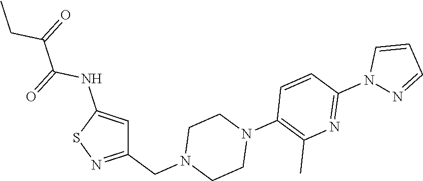 [SMILES: CCC(=O)C(=O)Nc1cc(CN2CCN(c3ccc(-n4cccn4)nc3C)CC2)ns1] | N-(3-((4-(2-methyl-6-(1H-pyrazol-1- yl)pyridin-3-yl)piperazin-1- yl)methyl)isothiazol-5-yl)-2- oxobutanamide |
| 353 | 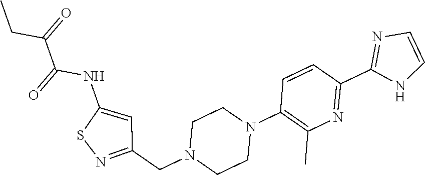 [SMILES: CCC(=O)C(=O)Nc1cc(CN2CCN(c3ccc(-c4ncc[nH]4)nc3C)CC2)ns1] | N-(3-((4-(6-(1H-imidazol-2-yl)-2- methylpyridin-3-yl)piperazin-1- yl)methyl)isothiazol-5-yl)-2- oxobutanamide |
| 354 | 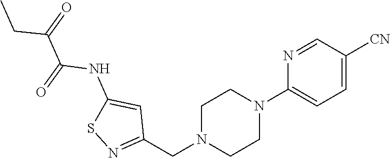 [SMILES: CCC(=O)C(=O)Nc1cc(CN2CCN(c3ccc(C#N)cn3)CC2)ns1] | N-(3-((4-(5-cyanopyridin-2-yl)piperazin- 1-yl)methyl)isothiazol-5-yl)-2- oxobutanamide |
| 355 | 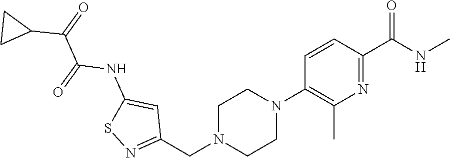 [SMILES: CNC(=O)c1ccc(N2CCN(Cc3cc(NC(=O)C(=O)C4CC4)sn3)CC2)c(C)n1] | 5-(4-((5-(2-cyclopropyl-2- oxoacetamido)isothiazol-3- yl)methyl)piperazin-1-yl)-N,6- dimethylpicolinamide |
| 356 | 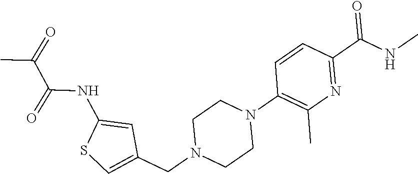 [SMILES: CNC(=O)c1ccc(N2CCN(Cc3csc(NC(=O)C(C)=O)c3)CC2)c(C)n1] | N,6-dimethyl-5-(4-((5-(2- oxopropanamido)thiophen-3- yl)methyl)piperazin-1-yl)picolinamide |
| 357 | 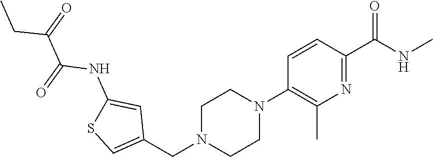 [SMILES: CCC(=O)C(=O)Nc1cc(CN2CCN(c3ccc(C(=O)NC)nc3C)CC2)cs1] | N,6-dimethyl-5-(4-((5-(2- oxobutanamido)thiophen-3- yl)methyl)piperazin-1-yl)picolinamide |
| 358 | 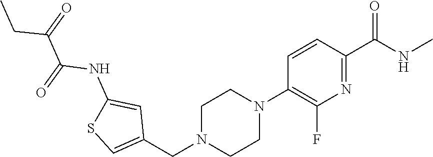 [SMILES: CCC(=O)C(=O)Nc1cc(CN2CCN(c3ccc(C(=O)NC)nc3F)CC2)cs1] | 6-fluoro-N-methyl-5-(4-((5-(2- oxobutanamido)thiophen-3- yl)methyl)piperazin-1-yl)picolinamide |
| 359 | 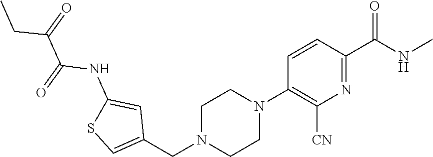 [SMILES: CCC(=O)C(=O)Nc1cc(CN2CCN(c3ccc(C(=O)NC)nc3C#N)CC2)cs1] | 6-cyano-N-methyl-5-(4-((5-(2- oxobutanamido)thiophen-3- yl)methyl)piperazin-1-yl)picolinamide |
| 360 | 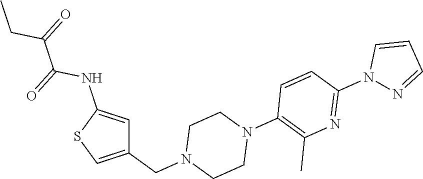 [SMILES: CCC(=O)C(=O)Nc1cc(CN2CCN(c3ccc(-n4cccn4)nc3C)CC2)cs1] | N-(4-((4-(2-methyl-6-(1H-pyrazol-]- yl)pyridin-3-yl)piperazin-1- yl)methyl)thiophen-2-yl)-2- oxobutanamide |
| 361 | 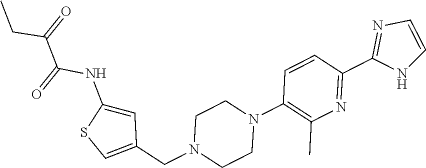 [SMILES: CCC(=O)C(=O)Nc1cc(CN2CCN(c3ccc(-c4ncc[nH]4)nc3C)CC2)cs1] | N-(4-((4-(6-(1H-imidazol-2-yl)-2- methylpyridin-3-yl)piperazin-1- yl)methyl)thiophen-2-yl)-2- oxobutanamide |
| 362 | 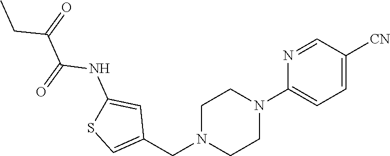 [SMILES: CCC(=O)C(=O)Nc1cc(CN2CCN(c3ccc(C#N)cn3)CC2)cs1] | N-(4-((4-(5-cyanopyridin-2-yl)piperazin- 1-yl)methyl)thiophen-2-yl)-2- oxobutanamide |
| 363 | 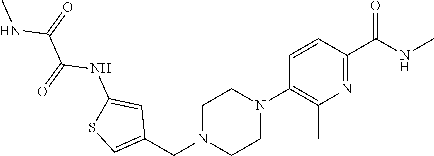 | N1-methyl-N2-(4-((4-(2-methyl-6- (methylcarbamoyl)pyridin-3-yl)piperazin- 1-yl)methyl)thiophen-2-yl)oxalamide |
| 364 | 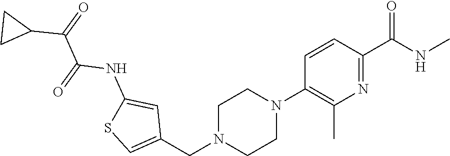 [SMILES: CNC(=O)c1ccc(N2CCN(Cc3csc(NC(=O)C(=O)C4CC4)c3)CC2)c(C)n1] | 5-(4-((5-(2-cyclopropyl-2- oxoacetamido)thiophen-3- yl)methyl)piperazin-1-yl)-N,6- dimethylpicolinamide |
| 365 | 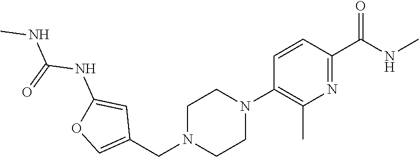 | N,6-dimethyl-5-(4-((5-(3- methylureido)furan-3- yl)methyl)piperazin-1-yl)picolinamide |
| 366 | 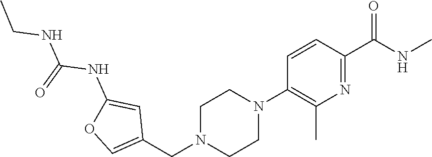 [SMILES: CCNC(=O)Nc1cc(CN2CCN(c3ccc(C(=O)NC)nc3C)CC2)co1] | 5-(4-((5-(3-ethylureido)furan-3- yl)methyl)piperazin-1-yl)-N,6- dimethylpicolinamide |
| 367 | 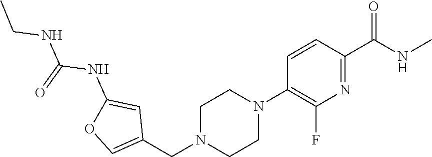 [SMILES: CCNC(=O)Nc1cc(CN2CCN(c3ccc(C(=O)NC)nc3F)CC2)co1] | 5-(4-((5-(3-ethylureido)furan-3- yl)methyl)piperazin-1-yl)-6-fluoro-N- methylpicolinamide |
| 368 | 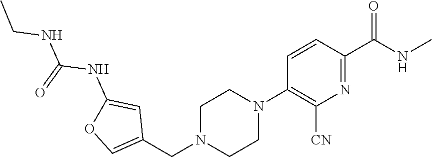 [SMILES: CCNC(=O)Nc1cc(CN2CCN(c3ccc(C(=O)NC)nc3C#N)CC2)co1] | 6-cyano-5-(4-((5-(3-ethylureido)furan-3- yl)methyl)piperazin-1-yl)-N- methylpicolinamide |
| 369 | 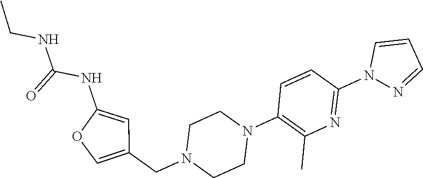 [SMILES: CCNC(=O)Nc1cc(CN2CCN(c3ccc(-n4cccn4)nc3C)CC2)co1] | 1-ethyl-3-(4-((4-(2-methyl-6-(1H- pyrazol-1-yl)pyridin-3-yl)piperazin-1- yl)methyl)furan-2-yl)urea |
| 370 | 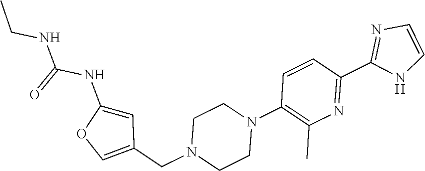 [SMILES: CCNC(=O)Nc1cc(CN2CCN(c3ccc(-c4ncc[nH]4)nc3C)CC2)co1] | 1-(4-((4-(6-(1H-imidazol-2-yl)-2- methylpyridin-3-yl)piperazin-1- yl)methyl)furan-2-yl)-3-ethylurea |
| 371 | 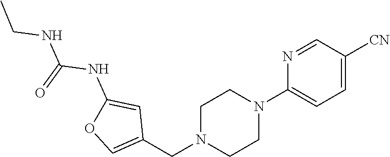 [SMILES: CCNC(=O)Nc1cc(CN2CCN(c3ccc(C#N)cn3)CC2)co1] | 1-(4-((4-(5-cyanopyridin-2-yl)piperazin- 1-yl)methyl)furan-2-yl)-3-ethylurea |
| 372 | 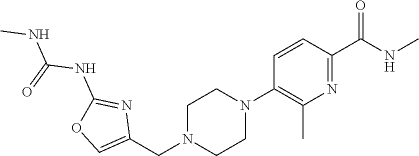 | N,6-dimethyl-5-(4-((2-(3- methylureido)oxazol-4- yl)methyl)piperazin-1-yl)picolinamide |
| 373 | 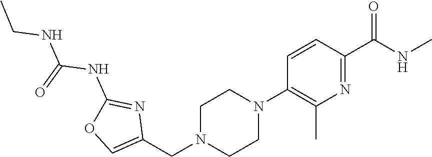 [SMILES: CCNC(=O)Nc1nc(CN2CCN(c3ccc(C(=O)NC)nc3C)CC2)co1] | 5-(4-((2-(3-ethylureido)oxazol-4- yl)methyl)piperazin-1-yl)-N,6- dimethylpicolinamide |
| 374 | 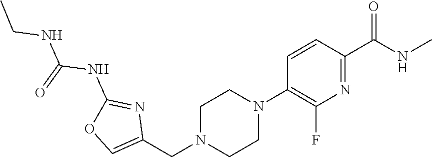 [SMILES: CCNC(=O)Nc1nc(CN2CCN(c3ccc(C(=O)NC)nc3F)CC2)co1] | 5-(4-((2-(3-ethylureido)oxazol-4- yl)methyl)piperazin-1-yl)-6-fluoro-N- methylpicolinamide |
| 375 | 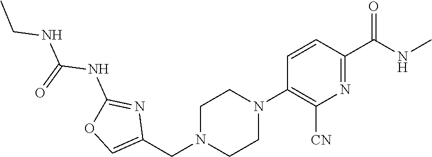 [SMILES: CCNC(=O)Nc1nc(CN2CCN(c3ccc(C(=O)NC)nc3C#N)CC2)co1] | 6-cyano-5-(4-((2-(3-ethylureido)oxazol-4- yl)methyl)piperazin-1-yl)-N- methylpicolinamide |
| 376 | 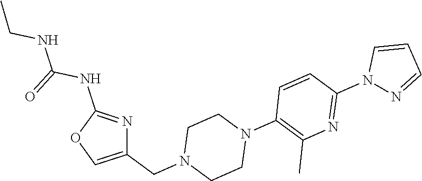 [SMILES: CCNC(=O)Nc1nc(CN2CCN(c3ccc(-n4cccn4)nc3C)CC2)co1] | 1-ethyl-3-(4-((4-(2-methyl-6-(1H- pyrazol-1-yl)pyridin-3-yl)piperazin-1- yl)methyl)oxazol-2-yl)urea |
| 377 | 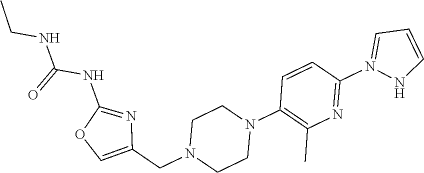 | 1-(4-((4-(6-(1H-imidazol-2-yl)-2- methylpyridin-3-yl)piperazin-1- yl)methyl)oxazol-2-yl)-3-ethylurea |
| 378 | 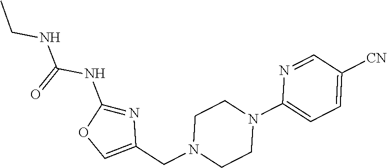 [SMILES: CCNC(=O)Nc1nc(CN2CCN(c3ccc(C#N)cn3)CC2)co1] | 1-(4-((4-(5-cyanopyridin-2-yl)piperazin- 1-yl)methyl)oxazol-2-yl)-3-ethylurea |
| 379 | 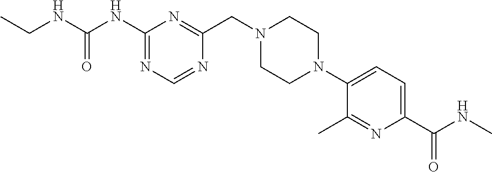 [SMILES: CCNC(=O)Nc1ncnc(CN2CCN(c3ccc(C(=O)NC)nc3C)CC2)n1] | 5-(4-((4-(3-ethylureido)-1,3,5-triazin-2- yl)methyl)piperazin-1-yl)-N,6- dimethylpicolinamide |
| 380 | 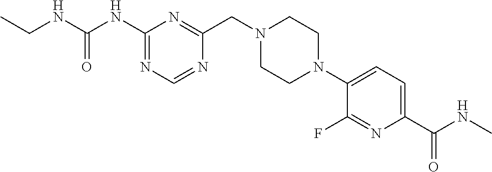 [SMILES: CCNC(=O)Nc1ncnc(CN2CCN(c3ccc(C(=O)NC)nc3F)CC2)n1] | 5-(4-((4-(3-ethylureido)-1,3,5-triazin-2- yl)methyl)piperazin-1-yl)-6-fluoro-N- methylpicolinamide |
| 381 | 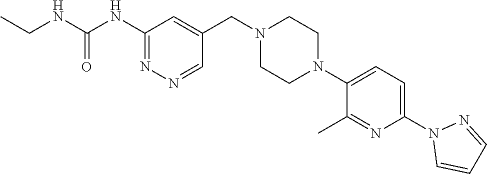 [SMILES: CCNC(=O)Nc1cc(CN2CCN(c3ccc(-n4cccn4)nc3C)CC2)cnn1] | 1-ethyl-3-(5-((4-(2-methyl-6-(1H- pyrazol-1-yl)pyridin-3-yl)piperazin-1- yl)methyl)pyridazin-3-yl)urea |
| 382 | 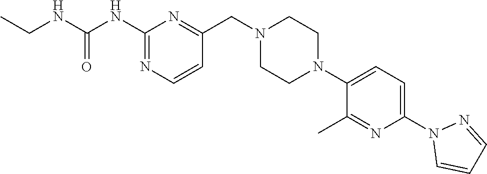 [SMILES: CCNC(=O)Nc1nccc(CN2CCN(c3ccc(-n4cccn4)nc3C)CC2)n1] | 1-ethyl-3-(4-((4-(2-methyl-6-(1H- pyrazol-1-yl)pyridin-3-yl)piperazin-1- yl)methyl)pyrimidin-2-yl)urea |
| 383 | 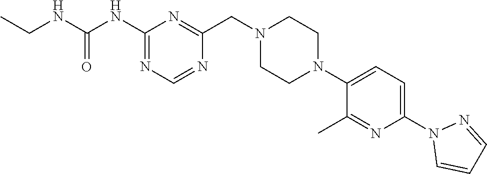 [SMILES: CCNC(=O)Nc1ncnc(CN2CCN(c3ccc(-n4cccn4)nc3C)CC2)n1] | 1-ethyl-3-(4-((4-(2-methyl-6-(1H- pyrazol-1-yl)pyridin-3-yl)piperazin-1- yl)methyl)-1,3,5-triazin-2-yl)urea |
| 384 | 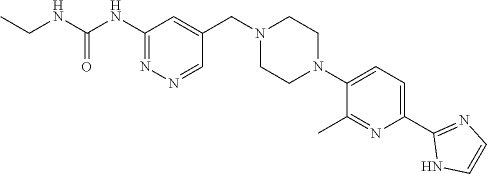 [SMILES: CCNC(=O)Nc1cc(CN2CCN(c3ccc(-c4ncc[nH]4)nc3C)CC2)cnn1] | 1-(5-((4-(6-(1H-imidazol-2-yl)-2- methylpyridin-3-yl)piperazin-1- yl)methyl)pyridazin-3-yl)-3-ethylurea |
| 385 | 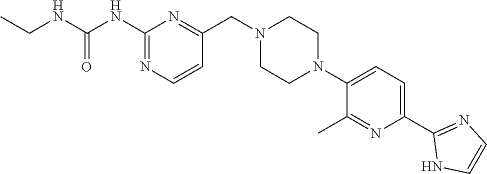 [SMILES: CCNC(=O)Nc1nccc(CN2CCN(c3ccc(-c4ncc[nH]4)nc3C)CC2)n1] | 1-(4-((4-(6-(1H-imidazol-2-yl)-2- methylpyridin-3-yl)piperazin-1- yl)methyl)pyrimidin-2-yl)-3-ethylurea |
| 386 | 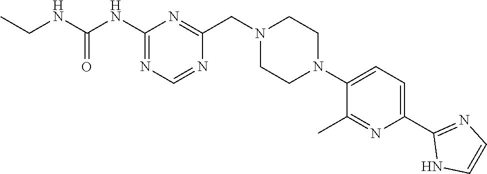 [SMILES: CCNC(=O)Nc1ncnc(CN2CCN(c3ccc(-c4ncc[nH]4)nc3C)CC2)n1] | 1-(4-((4-(6-(1H-imidazol-2-yl)-2- methylpyridin-3-yl)piperazin-1- yl)methyl)-1,3,5-triazin-2-yl)-3-ethylurea |
| 387 | 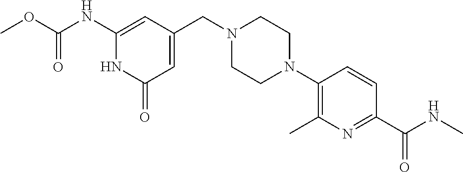 | methyl (4-((4-(2-methyl-6- (methylcarbamoyl)pyridin-3-yl)piperazin- 1-yl)methyl)-6-oxo-1,6-dihydropyridin-2- yl)carbamate |
| 388 | 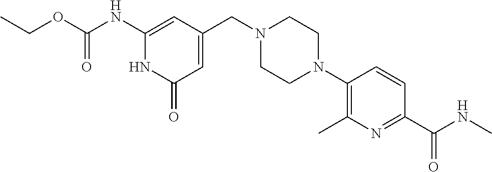 [SMILES: CCOC(=O)Nc1cc(CN2CCN(c3ccc(C(=O)NC)nc3C)CC2)cc(=O)[nH]1] | ethyl (4-((4-(2-methyl-6- (methylcarbamoyl)pyridin-3-yl)piperazin- 1-yl)methyl)-6-oxo-1,6-dihydropyridin-2- yl)carbamate |
| 389 | 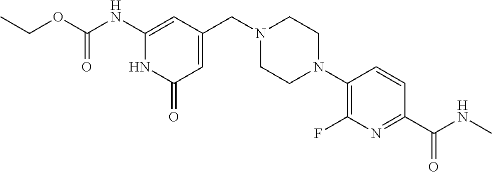 [SMILES: CCOC(=O)Nc1cc(CN2CCN(c3ccc(C(=O)NC)nc3F)CC2)cc(=O)[nH]1] | ethyl (4-((4-(2-fluoro-6. (methylcarbamoyl)pyridin-3-yl)piperazin- 1-yl)methyl)-6-oxo-1,6-dihydropyridin-2- yl)carbamate |
| 390 | 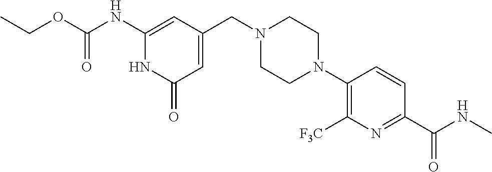 [SMILES: CCOC(=O)Nc1cc(CN2CCN(c3ccc(C(=O)NC)nc3C(F)(F)F)CC2)cc(=O)[nH]1] | ethyl (4-((4-(6-(methylcarbamoyl)-2- (trifluoromethyl)pyridin-3-yl)piperazin-1- yl)methyl)-6-oxo-1,6-dihydropyridin-2- yl)carbamate |
| 391 | 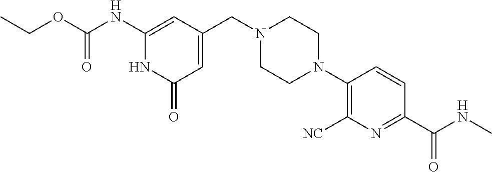 [SMILES: CCOC(=O)Nc1cc(CN2CCN(c3ccc(C(=O)NC)nc3C#N)CC2)cc(=O)[nH]1] | ethyl (4-((4-(2-cyano-6- (methylcarbamoyl)pyridin-3-yl)piperazin- 1-yl)methyl)-6-oxo-1,6-dihydropyridin-2- yl)carbamate |
| 392 | 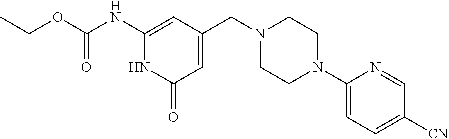 [SMILES: CCOC(=O)Nc1cc(CN2CCN(c3ccc(C#N)cn3)CC2)cc(=O)[nH]1] | ethyl (4-((4-(5-cyanopyridin-2- yl)piperazin-1-yl)methyl)-6-oxo-1,6- dihydropyridin-2-yl)carbamate |
| 393 | 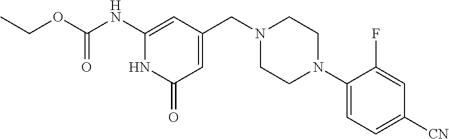 [SMILES: CCOC(=O)Nc1cc(CN2CCN(c3ccc(C#N)cc3F)CC2)cc(=O)[nH]1] | ethyl (4-((4-(4-cyano-2- fluorophenyl)piperazin-1-yl)methyl)-6- oxo-1,6-dihydropyridin-2-yl)carbamate |
| 394 | 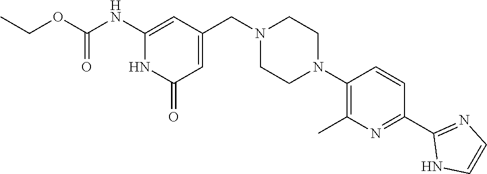 [SMILES: CCOC(=O)Nc1cc(CN2CCN(c3ccc(-c4ncc[nH]4)nc3C)CC2)cc(=O)[nH]1] | ethyl (4-((4-(6-(1H-imidazol-2-yl)-2- methylpyridin-3-yl)piperazin-1- yl)methyl)-6-oxo-1,6-dihydropyridin-2- yl)carbamate |
| 395 | 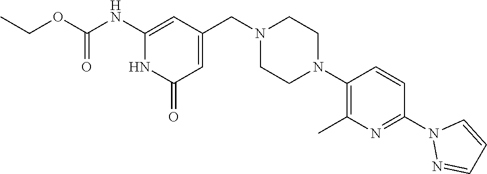 [SMILES: CCOC(=O)Nc1cc(CN2CCN(c3ccc(-n4cccn4)nc3C)CC2)cc(=O)[nH]1] | ethyl (4-((4-(2-methyl-6-(1H-pyrazol-1- yl)pyridin-3-yl)piperazin-1-yl)methyl)-6- oxo-1,6-dihydropyridin-2-yl)carbamate |
| 396 | 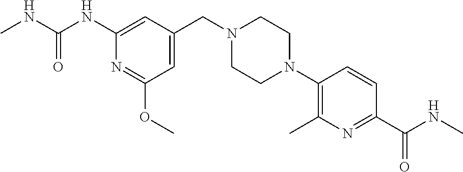 | 5-(4-((2-methoxy-6-(3- methylureido)pyridin-4- yl)methyl)piperazin-1-yl)-N,6- dimethylpicolinamide |
| 397 | 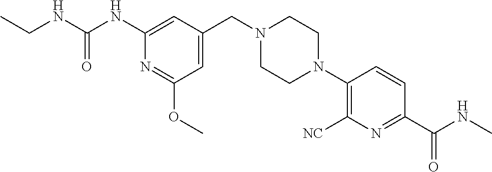 [SMILES: CCNC(=O)Nc1cc(CN2CCN(c3ccc(C(=O)NC)nc3C#N)CC2)cc(OC)n1] | 6-cyano-5-(4-((2-(3-ethylureido)-6- methoxypyridin-4-yl)methyl)piperazin-1- yl)-N-methylpicolinamide |
| 398 | 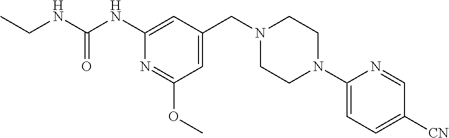 [SMILES: CCNC(=O)Nc1cc(CN2CCN(c3ccc(C#N)cn3)CC2)cc(OC)n1] | 1-(4-((4-(5-cyanopyridin-2-yl)piperazin- 1-yl)methyl)-6-methoxypyridin-2-yl)-3- ethylurea |
| 399 | 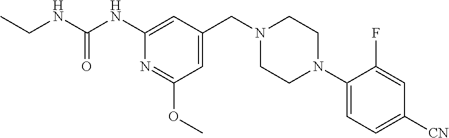 [SMILES: CCNC(=O)Nc1cc(CN2CCN(c3ccc(C#N)cc3F)CC2)cc(OC)n1] | 1-(4-((4-(4-cyano-2- fluorophenyl)piperazin-1-yl)methyl)-6- methoxypyridin-2-yl)-3-ethylurea |
| 400 | 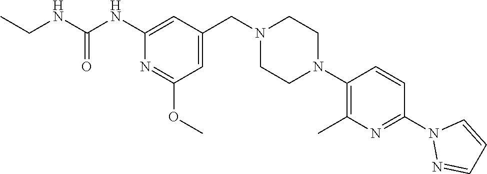 [SMILES: CCNC(=O)Nc1cc(CN2CCN(c3ccc(-n4cccn4)nc3C)CC2)cc(OC)n1] | 1-ethyl-3-(6-methoxy-4-((4-(2-methyl-6- (1H-pyrazol-1-yl)pyridin-3-yl)piperazin- 1-yl)methyl)pyridin-2-yl)urea |
| 401 | 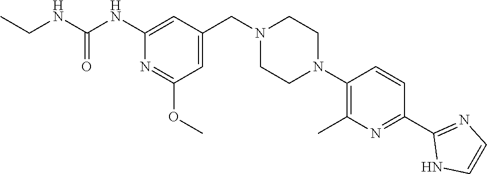 [SMILES: CCNC(=O)Nc1cc(CN2CCN(c3ccc(-c4ncc[nH]4)nc3C)CC2)cc(OC)n1] | 1-(4-((4-(6-(1H-imidazol-2-yl)-2- methylpyridin-3-yl)piperazin-1- yl)methyl)-6-methoxypyridin-2-yl)-3- ethylurea |
| 402 | 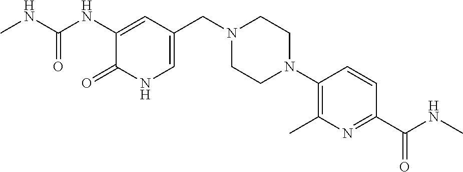 | N,6-dimethyl-5-(4-((5-(3-methylureido)- 6-oxo-1,6-dihydropyridin-3- yl)methyl)piperazin-1-yl)picolinamide |
| 403 | 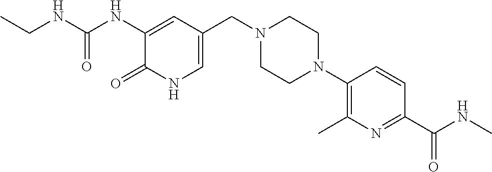 [SMILES: CCNC(=O)Nc1cc(CN2CCN(c3ccc(C(=O)NC)nc3C)CC2)c[nH]c1=O] | 5-(4-((5-(3-ethylureido)-6-oxo-1,6- dihydropyridin-3-yl)methyl)piperazin-1- yl)-N,6-dimethylpicolinamide |
| 404 | 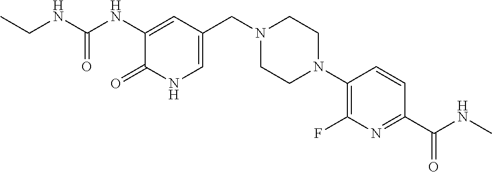 [SMILES: CCNC(=O)Nc1cc(CN2CCN(c3ccc(C(=O)NC)nc3F)CC2)c[nH]c1=O] | 5-(4-((5-(3-ethylureido)-6-oxo-1,6- dihydropyridin-3-yl)methyl)piperazin-1- yl)-6-fluoro-N-methylpicolinamide |
| 405 | 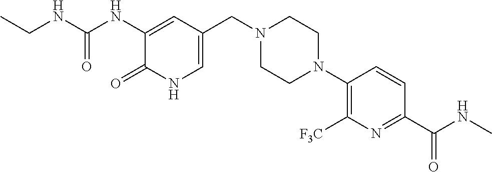 [SMILES: CCNC(=O)Nc1cc(CN2CCN(c3ccc(C(=O)NC)nc3C(F)(F)F)CC2)c[nH]c1=O] | S-(4-((5-(3-ethylureido)-6-oxo-1,6- dihydropyridin-3-yl)methyl)piperazin-1- yl)-N-methyl-6- (trifluoromethyl)picolinamide |
| 406 | 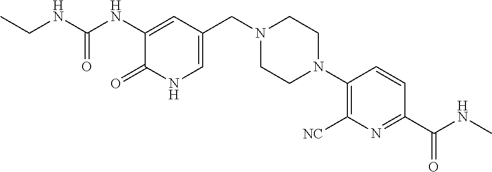 [SMILES: CCNC(=O)Nc1cc(CN2CCN(c3ccc(C(=O)NC)nc3C#N)CC2)c[nH]c1=O] | 6-cyano-5-(4-((5-(3-ethylureido)-6-oxo- 1,6-dihydropyridin-3- yl)methyl)piperazin-1-yl)-N- methylpicolinamide |
| 407 | 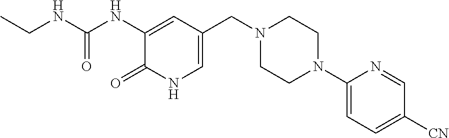 [SMILES: CCNC(=O)Nc1cc(CN2CCN(c3ccc(C#N)cn3)CC2)c[nH]c1=O] | 6-cyano-5-(4-((5-(3-ethylureido)-6-oxo- 1,6-dihydropyridin-3- yl)methyl)piperazin-1-yl)-N- methylpicolinamide |
| 408 | 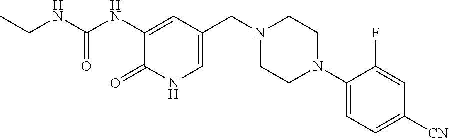 [SMILES: CCNC(=O)Nc1cc(CN2CCN(c3ccc(C#N)cc3F)CC2)c[nH]c1=O] | 1-(5-((4-(4-cyano-2- fluorophenyl)piperazin-1-yl)methyl)-2- oxo-1,2-dihydropyridin-3-yl)-3-ethylurea |
| 409 | 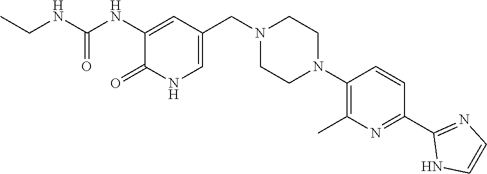 [SMILES: CCNC(=O)Nc1cc(CN2CCN(c3ccc(-c4ncc[nH]4)nc3C)CC2)c[nH]c1=O] | 1-(5-((4-(6-(1H-imidazol-2-yl)-2- methylpyridin-3-yl)piperazin-1- yl)methyl)-2-oxo-1,2-dihydropyridin-3- yl)-3-ethylurea |
| 410 | 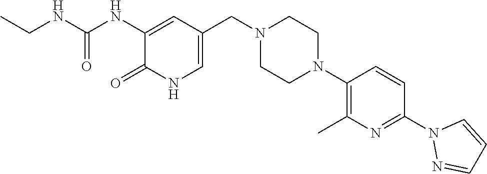 [SMILES: CCNC(=O)Nc1cc(CN2CCN(c3ccc(-n4cccn4)nc3C)CC2)c[nH]c1=O] | 1-ethyl-3-(5-((4-(2-methyl-6-(1H- pyrazol-1-yl)pyridin-3-yl)piperazin-1- yl)methyl)-2-oxo-1,2-dihydropyridin-3- yl)urea |
| 411 | 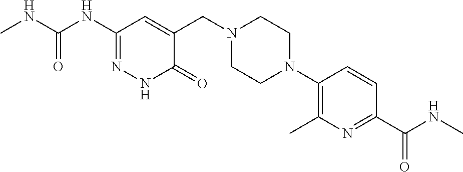 | N,6-dimethyl-5-(4-((6-(3-methylureido)- 3-oxo-2,3-dihydropyridazin-4- yl)methyl)piperazin-1-yl)picolinamide |
| 412 | 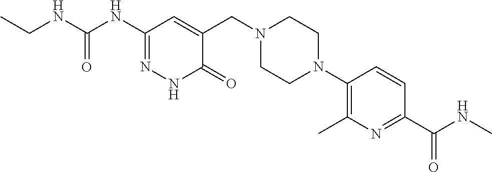 [SMILES: CCNC(=O)Nc1cc(CN2CCN(c3ccc(C(=O)NC)nc3C)CC2)c(=O)[nH]n1] | 5-(4-((6-(3-ethylureido)-3-oxo-2,3- dihydropyridazin-4-yl)methyl)piperazin- 1-yl)-N,6-dimethylpicolinamide |
| 413 | 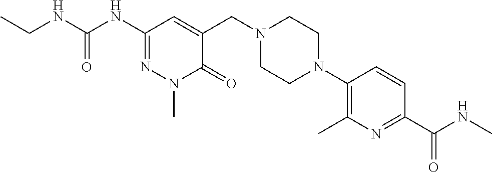 [SMILES: CCNC(=O)Nc1cc(CN2CCN(c3ccc(C(=O)NC)nc3C)CC2)c(=O)n(C)n1] | 5-(4-((6-(3-ethylureido)-2-methyl-3-oxo- 2,3-dihydropyridazin-4- yl)methyl)piperazin-1-yl)-N,6- dimethylpicolinamide |
| 414 | 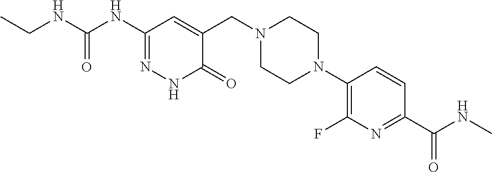 [SMILES: CCNC(=O)Nc1cc(CN2CCN(c3ccc(C(=O)NC)nc3F)CC2)c(=O)[nH]n1] | 5-(4-((6-(3-ethylureido)-3-oxo-2,3- dihydropyridazin-4-yl)methyl)piperazin- 1-yl)-6-fluoro-N-methylpicolinamide |
| 415 | 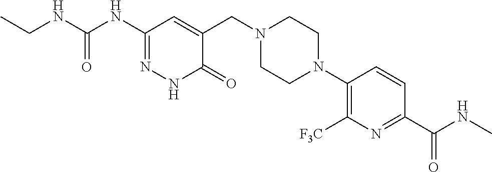 [SMILES: CCNC(=O)Nc1cc(CN2CCN(c3ccc(C(=O)NC)nc3C(F)(F)F)CC2)c(=O)[nH]n1] | 5-(4-((6-(3-ethylureido)-3-oxo-2,3- dihydropyridazin-4-yl)methyl)piperazin- 1-yl)-N-methyl-6- (trifluoromethyl)picolinamide |
| 416 | 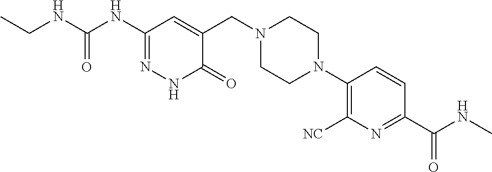 [SMILES: CCNC(=O)Nc1cc(CN2CCN(c3ccc(C(=O)NC)nc3C#N)CC2)c(=O)[nH]n1] | 6-cyano-5-(4-((6-(3-ethylureido)-3-oxo- 2,3-dihydropyridazin-4- yl)methyl)piperazin-1-yl)-N- methylpicolinamide |
| 417 | 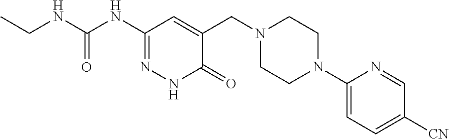 [SMILES: CCNC(=O)Nc1cc(CN2CCN(c3ccc(C#N)cn3)CC2)c(=O)[nH]n1] | 1-(5-((4-(5-cyanopyridin-2-yl)piperazin- 1-yl)methyl)-6-oxo-1,6-dihydropyridazin- 3-yl)-3-ethylurea |
| 418 | 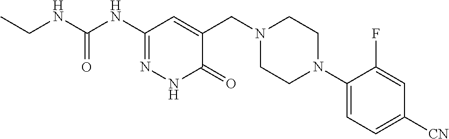 [SMILES: CCNC(=O)Nc1cc(CN2CCN(c3ccc(C#N)cc3F)CC2)c(=O)[nH]n1] | 1-(5-((4-(4-cyano-2- fluorophenyl)piperazin-1-yl)methyl)-6- oxo-1,6-dihydropyridazin-3-yl)-3- ethylurea |
| 419 | 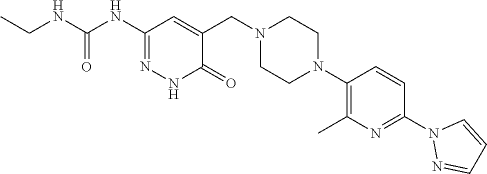 [SMILES: CCNC(=O)Nc1cc(CN2CCN(c3ccc(-n4cccn4)nc3C)CC2)c(=O)[nH]n1] | 1-ethyl-3-(5-((4-(2-methyl-6-(1H- pyrazol-1-yl)pyridin-3-yl)piperazin-1- yl)methyl)-6-oxo-1,6-dihydropyridazin-3- yl)urea |
| 420 | 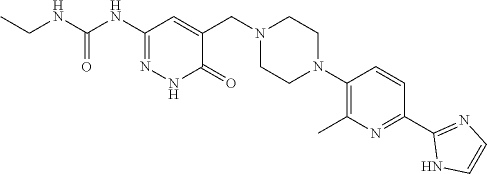 [SMILES: CCNC(=O)Nc1cc(CN2CCN(c3ccc(-c4ncc[nH]4)nc3C)CC2)c(=O)[nH]n1] | 1-(5-((4-(6-(1H-imidazol-2-yl)-2- methylpyridin-3-yl)piperazin-1- yl)methyl)-6-oxo-1,6-dihydropyridazin-3- yl)-3-ethylurea |
| 421 | 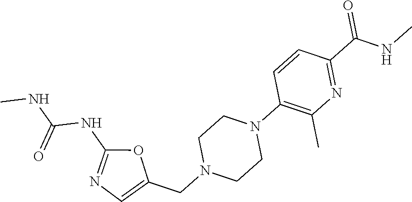 | N,6-dimethyl-5-(4-((2-(3- methylureido)oxazol-5- yl)methyl)piperazin-1-yl)picolinamide |
| 422 | 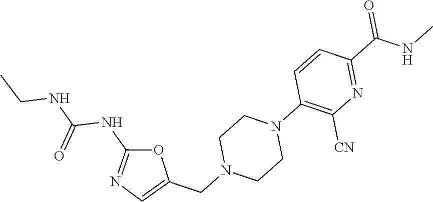 [SMILES: CCNC(=O)Nc1ncc(CN2CCN(c3ccc(C(=O)NC)nc3C#N)CC2)o1] | 6-cyano-5-(4-((2-(3-ethylureido)oxazol-5- yl)methyl)piperazin-1-yl)-N- methylpicolinamide |
| 423 | 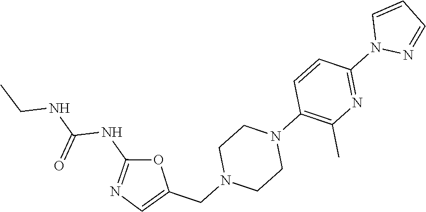 [SMILES: CCNC(=O)Nc1ncc(CN2CCN(c3ccc(-n4cccn4)nc3C)CC2)o1] | 1-ethyl-3-(5-((4-(2-methyl-6-(1H- pyrazol-1-yl)pyridin-3-yl)piperazin-1- yl)methyl)oxazol-2-yl)urea |
| 424 | 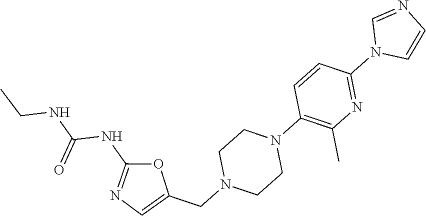 [SMILES: CCNC(=O)Nc1ncc(CN2CCN(c3ccc(-n4ccnc4)nc3C)CC2)o1] | 1-(5-((4-(6-(1H-imidazol-1-yl)-2- methylpyridin-3-yl)piperazin-1- yl)methyl)oxazol-2-yl)-3-ethylurea |
| 425 | 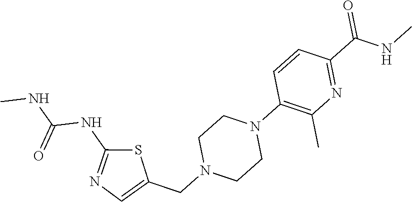 | N,6-dimethyl-5-(4-((2-(3- methylureido)thiazol-5- yl)methyl)piperazin-1-yl)picolinamide |
| 426 | 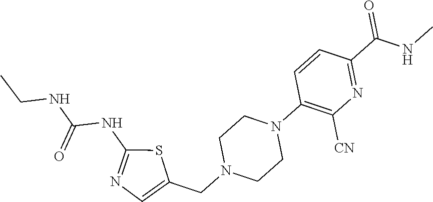 [SMILES: CCNC(=O)Nc1ncc(CN2CCN(c3ccc(C(=O)NC)nc3C#N)CC2)s1] | 6-cyano-5-(4-((2-(3-ethylureido)thiazol- 5-yl)methyl)piperazin-1-yl)-N- methylpicolinamide |
| 427 | 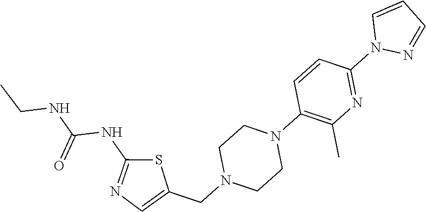 [SMILES: CCNC(=O)Nc1ncc(CN2CCN(c3ccc(-n4cccn4)nc3C)CC2)s1] | 1-ethyl-3-(5-((4-(2-methyl-6-(1H- pyrazol-1-yl)pyridin-3-yl)piperazin-1- yl)methyl)thiazol-2-yl)urea |
| 428 | 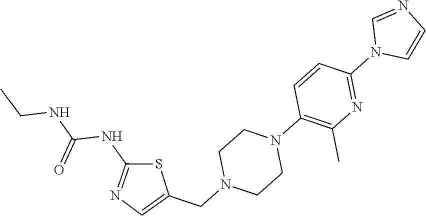 [SMILES: CCNC(=O)Nc1ncc(CN2CCN(c3ccc(-n4ccnc4)nc3C)CC2)s1] | 1-(5-((4-(6-(1H-imidazol-1-yl)-2- methylpyridin-3-yl)piperazin-1- yl)methyl)thiazol-2-yl)-3-ethylurea |
| 429 | 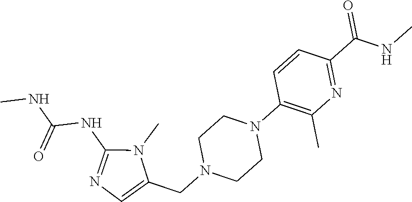 | N,6-dimethyl-S-(4-((1-methyl-2-(3- methylureido)-1H-imidazol-5- yl)methyl)piperazin-1-yl)picolinamide |
| 430 | 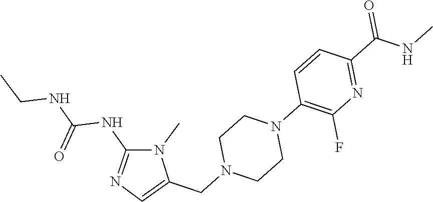 [SMILES: CCNC(=O)Nc1ncc(CN2CCN(c3ccc(C(=O)NC)nc3F)CC2)n1C] | 5-(4-((2-(3-ethylureido)-1-methyl-1H- imidazol-5-yl)methyl)piperazin-1-yl)-6- fluoro-N-methylpicolinamide |
| 431 | 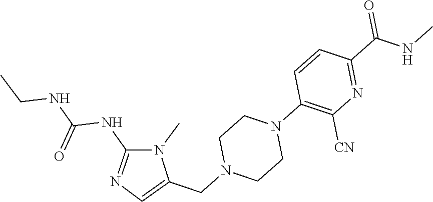 [SMILES: CCNC(=O)Nc1ncc(CN2CCN(c3ccc(C(=O)NC)nc3C#N)CC2)n1C] | 6-cyano-5-(4-((2-(3-ethylureido)-1- methyl-1H-imidazol-5- yl)methyl)piperazin-1-yl)-N- methylpicolinamide |
| 432 | 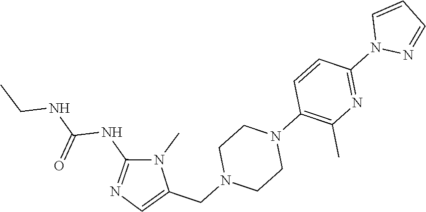 [SMILES: CCNC(=O)Nc1ncc(CN2CCN(c3ccc(-n4cccn4)nc3C)CC2)n1C] | 1-ethyl-3-(1-methyl-5-((4-(2-methyl-6- (1H-pyrazol-1-yl)pyridin-3-yl)piperazin- 1-yl)methyl)-1H-imidazol-2-yl)urea |
| 433 | 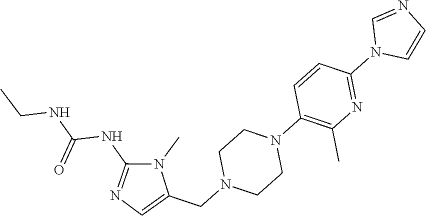 [SMILES: CCNC(=O)Nc1ncc(CN2CCN(c3ccc(-n4ccnc4)nc3C)CC2)n1C] | 1-(5-((4-(6-(1H-imidazol-1-yl)-2- methylpyridin-3-yl)piperazin-1- yl)methyl)-1-methyl-1H-imidazol-2-yl)- 3-ethylurea |
| 434 | 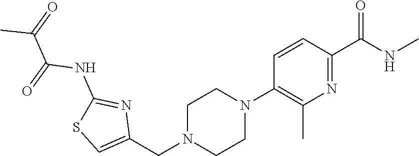 [SMILES: CNC(=O)c1ccc(N2CCN(Cc3csc(NC(=O)C(C)=O)n3)CC2)c(C)n1] | N,6-dimethyl-5-(4-((2-(2- oxopropanamido)thiazol-4- yl)methyl)piperazin-1-yl)picolinamide |
| 435 | 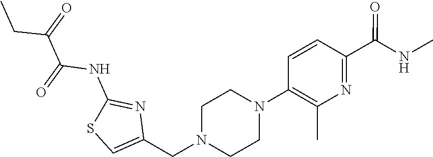 [SMILES: CCC(=O)C(=O)Nc1nc(CN2CCN(c3ccc(C(=O)NC)nc3C)CC2)cs1] | N,6-dimethyl-5-(4-((2-(2- oxobutanamido)thiazol-4- yl)methyl)piperazin-1-yl)picolinamide |
| 436 | 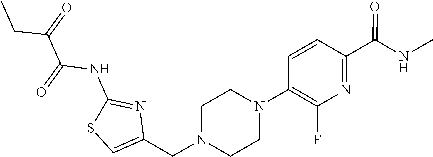 [SMILES: CCC(=O)C(=O)Nc1nc(CN2CCN(c3ccc(C(=O)NC)nc3F)CC2)cs1] | 6-fluoro-N-methyl-5-(4-((2-(2- oxobutanamido)thiazol-4- yl)methyl)piperazin-1-yl)picolinamide |
| 437 | 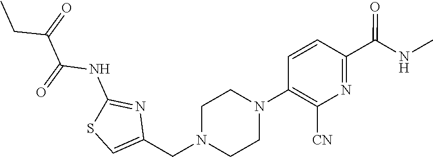 [SMILES: CCC(=O)C(=O)Nc1nc(CN2CCN(c3ccc(C(=O)NC)nc3C#N)CC2)cs1] | 6-cyano-N-methyl-5-(4-((2-(2- oxobutanamido)thiazol-4- yl)methyl)piperazin-1-yl)picolinamide |
| 438 | 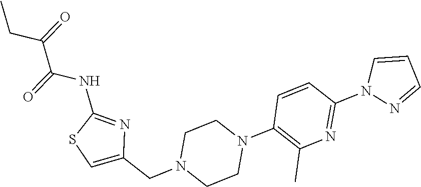 [SMILES: CCC(=O)C(=O)Nc1nc(CN2CCN(c3ccc(-n4cccn4)nc3C)CC2)cs1] | N-(4-((4-(2-methyl-6-(1H-pyrazol-1- yl)pyridin-3-yl)piperazin-1- yl)methyl)thiazol-2-yl)-2-oxobutanamide |
| 439 | 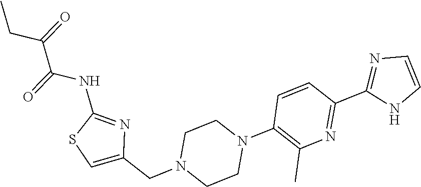 [SMILES: CCC(=O)C(=O)Nc1nc(CN2CCN(c3ccc(-c4ncc[nH]4)nc3C)CC2)cs1] | N-(4-((4-(6-(1H-imidazol-2-yl)-2- methylpyridin-3-yl)piperazin-1- yl)methyl)thiazol-2-yl)-2-oxobutanamide |
| 440 | 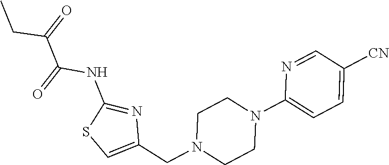 [SMILES: CCC(=O)C(=O)Nc1nc(CN2CCN(c3ccc(C#N)cn3)CC2)cs1] | N-(4-((4-(5-cyanopyridin-2-yl)piperazin- 1-yl)methyl)thiazol-2-yl)-2- oxobutanamide |
| 441 | 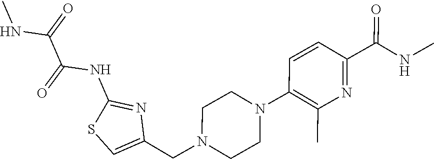 | N1-methyl-N2-(4-((4-(2-methyl-6- (methylcarbamoyl)pyridin-3-yl)piperazin- 1-yl)methyl)thiazol-2-yl)oxalamide |
| 442 | 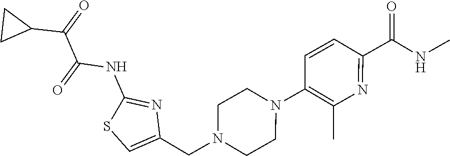 [SMILES: CNC(=O)c1ccc(N2CCN(Cc3csc(NC(=O)C(=O)C4CC4)n3)CC2)c(C)n1] | 5-(4-((2-(2-cyclopropyl-2- oxoacetamido)thiazol-4- yl)methyl)piperazin-1-yl)-N,6- dimethylpicolinamide |
| 443 | 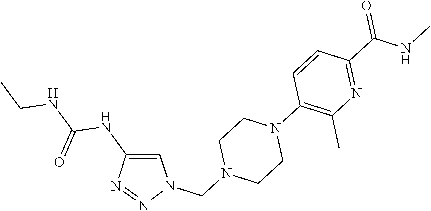 [SMILES: CCNC(=O)Nc1cn(CN2CCN(c3ccc(C(=O)NC)nc3C)CC2)nn1] | 5-(4-((4-(3-ethylureido)-1H-1,2,3-triazol- 1-yl)methyl)piperazin-1-yl)-N,6- dimethylpicolinamide |
| 444 | 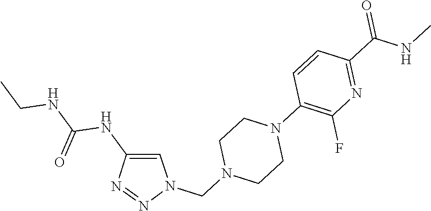 [SMILES: CCNC(=O)Nc1cn(CN2CCN(c3ccc(C(=O)NC)nc3F)CC2)nn1] | 5-(4-((4-(3-ethylureido)-1H-1,2,3-triazol- 1-yl)methyl)piperazin-1-yl)-6-fluoro-N- methylpicolinamide |
| 445 | 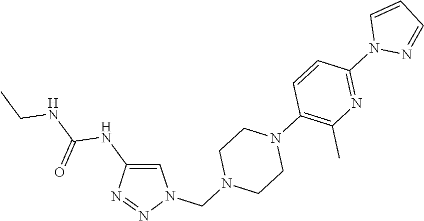 [SMILES: CCNC(=O)Nc1cn(CN2CCN(c3ccc(-n4cccn4)nc3C)CC2)nn1] | 1-ethyl-3-(1-((4-(2-methyl-6-(1H- pyrazol-1-yl)pyridin-3-yl)piperazin-1- yl)methyl)-1H-1,2,3-triazol-4-yl)urea |
| 446 | 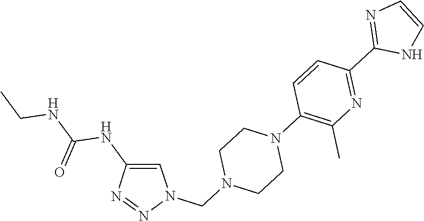 [SMILES: CCNC(=O)Nc1cn(CN2CCN(c3ccc(-c4ncc[nH]4)nc3C)CC2)nn1] | 1-(1-((4-(6-(1H-imidazol-2-yl)-2- methylpyridin-3-yl)piperazin-1- yl)methyl)-1H-1,2,3-triazol-4-yl)-3- ethylurea |
| 447 | 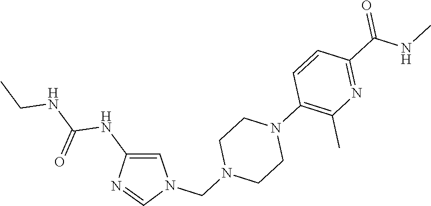 [SMILES: CCNC(=O)Nc1cn(CN2CCN(c3ccc(C(=O)NC)nc3C)CC2)cn1] | 5-(4-((4-(3-ethylureido)-1H-imidazol-1- yl)methyl)piperazin-1-yl)-N,6- dimethylpicolinamide |
| 448 | 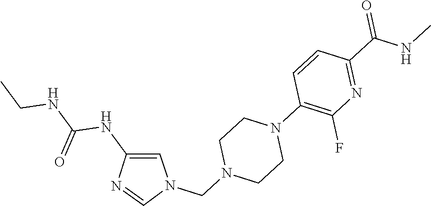 [SMILES: CCNC(=O)Nc1cn(CN2CCN(c3ccc(C(=O)NC)nc3F)CC2)cn1] | 5-(4-((4-(3-ethylureido)-1H-imidazol-1- yl)methyl)piperazin-1-yl)-6-fluoro-N- methylpicolinamide |
| 449 | 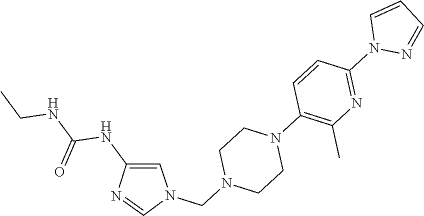 [SMILES: CCNC(=O)Nc1cn(CN2CCN(c3ccc(-n4cccn4)nc3C)CC2)cn1] | 1-ethyl-3-(1-((4-(2-methyl-6-(1H- pyrazol-1-yl)pyridin-3-yl)piperazin-1- yl)methyl)-1H-imidazol-4-yl)urea |
| 450 | 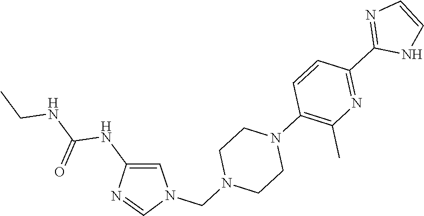 [SMILES: CCNC(=O)Nc1cn(CN2CCN(c3ccc(-c4ncc[nH]4)nc3C)CC2)cn1] | 1-(1-((4-(6-(1H-imidazol-2-yl)-2- methylpyridin-3-yl)piperazin-1- yl)methyl)-1H-imidazol-4-yl)-3-ethylurea |
| 451 | 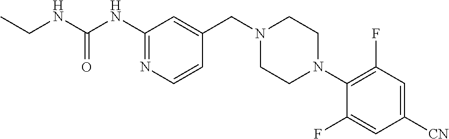 [SMILES: CCNC(=O)Nc1cc(CN2CCN(c3c(F)cc(C#N)cc3F)CC2)ccn1] | 1-(4-((4-(4-cyano-2,6- difluorophenyl)piperazin-1- yl)methyl)pyridin-2-yl)-3-ethylurea |
| 452 | 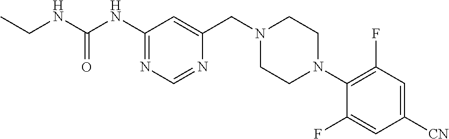 [SMILES: CCNC(=O)Nc1cc(CN2CCN(c3c(F)cc(C#N)cc3F)CC2)ncn1] | 1-(6-((4-(4-cyano-2,6- difluorophenyl)piperazin-1- yl)methyl)pyrimidin-4-yl)-3-ethylurea |
| 453 | 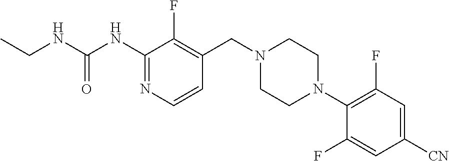 [SMILES: CCNC(=O)Nc1nccc(CN2CCN(c3c(F)cc(C#N)cc3F)CC2)c1F] | 1-(4-((4-(4-cyano-2,6- difluorophenyl)piperazin-1-yl)methyl)-3- fluoropyridin-2-yl)-3-ethylurea |
| 454 | 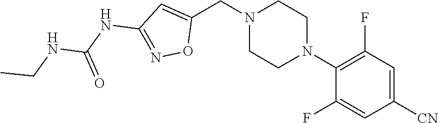 [SMILES: CCNC(=O)Nc1cc(CN2CCN(c3c(F)cc(C#N)cc3F)CC2)on1] | 1-(5-((4-(4-cyano-2,6- difluorophenyl)piperazin-1- yl)methyl)isoxazol-3-yl)-3-cthylurea |
| 455 | 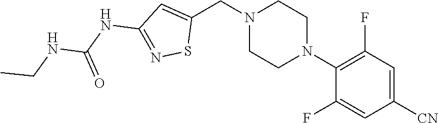 [SMILES: CCNC(=O)Nc1cc(CN2CCN(c3c(F)cc(C#N)cc3F)CC2)sn1] | 1-(5-((4-(4-cyano-2,6- difluorophenyl)piperazin-1- yl)methyl)isothiazol-3-yl)-3-ethylurea |
| 456 | 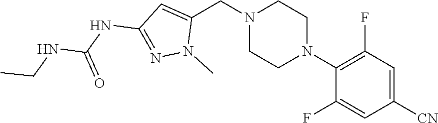 [SMILES: CCNC(=O)Nc1cc(CN2CCN(c3c(F)cc(C#N)cc3F)CC2)n(C)n1] | 1-(5-((4-(4-cyano-2,6- difluorophenyl)piperazin-1-yl)methyl)-1- methyl-1H-pyrazol-3-yl)-3-ethylurea |
| 457 | 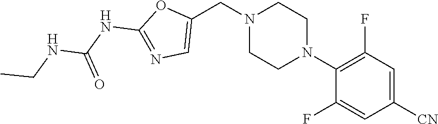 [SMILES: CCNC(=O)Nc1ncc(CN2CCN(c3c(F)cc(C#N)cc3F)CC2)o1] | 1-(5-((4-(4-cyano-2,6- difluorophenyl)piperazin-1- yl)methyl)oxazol-2-yl)-3-ethylurea |
| 458 | 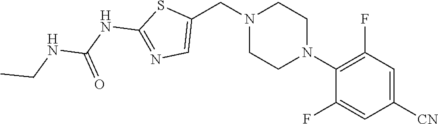 [SMILES: CCNC(=O)Nc1ncc(CN2CCN(c3c(F)cc(C#N)cc3F)CC2)s1] | 1-(5-((4-(4-cyano-2,6- difluorophenyl)piperazin-1- yl)methyl)thiazol-2-yl)-3-ethylurea |
| 459 | 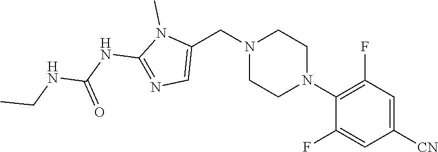 [SMILES: CCNC(=O)Nc1ncc(CN2CCN(c3c(F)cc(C#N)cc3F)CC2)n1C] | 1-(5-((4-(4-cyano-2,6- difluorophenyl)piperazin-1-yl)methyl)-1- methyl-1H-imidazol-2-yl)-3-ethylurea |
| 460 | 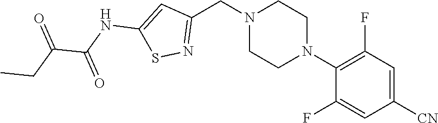 [SMILES: CCC(=O)C(=O)Nc1cc(CN2CCN(c3c(F)cc(C#N)cc3F)CC2)ns1] | N-(3-((4-(4-cyano-2,6- difluorophenyl)piperazin-1- yl)methyl)isothiazol-5-yl)-2- oxobutanamide |
| 461 | 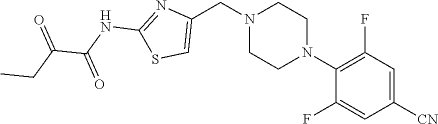 [SMILES: CCC(=O)C(=O)Nc1nc(CN2CCN(c3c(F)cc(C#N)cc3F)CC2)cs1] | N-(4-((4-(4-cyano-2,6- difluorophenyl)piperazin-1- yl)methyl)thiazol-2-yl)-2-oxobutanamide |
| 462 | 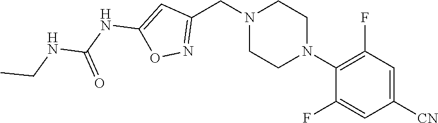 [SMILES: CCNC(=O)Nc1cc(CN2CCN(c3c(F)cc(C#N)cc3F)CC2)no1] | 1-(3-((4-(4-cyano-2,6- difluorophenyl)piperazin-1- yl)methyl)isoxazol-5-yl)-3-ethylurea |
| 463 | 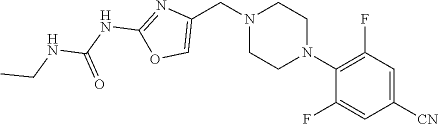 [SMILES: CCNC(=O)Nc1nc(CN2CCN(c3c(F)cc(C#N)cc3F)CC2)co1] | 1-(4-((4-(4-cyano-2,6- difluorophenyl)piperazin-1- yl)methyl)oxazol-2-yl)-3-ethylurea |
| 464 | 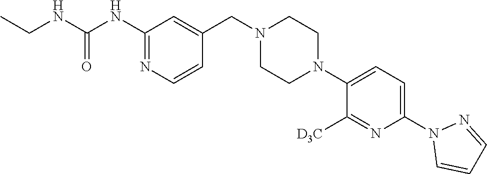 [SMILES: [2H]C([2H])([2H])c1nc(-n2cccn2)ccc1N1CCN(Cc2ccnc(NC(=O)NCC)c2)CC1] | 1-ethyl-3-(4-((4-(2-(methyl-d3)-6-(1H- pyrazol-1-yl)pyridin-3-yl)piperazin-1- yl)methyl)pyridin-2-yl)urea |
| 465 | 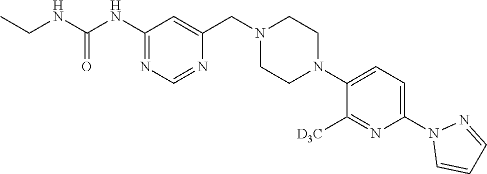 [SMILES: [2H]C([2H])([2H])c1nc(-n2cccn2)ccc1N1CCN(Cc2cc(NC(=O)NCC)ncn2)CC1] | 1-ethyl-3-(6-((4-(2-(methyl-d3)-6-(1H- pyrazol-1-yl)pyridin-3-yl)piperazin-1- yl)methyl)pyrimidin-4-yl)urea |
| 466 | 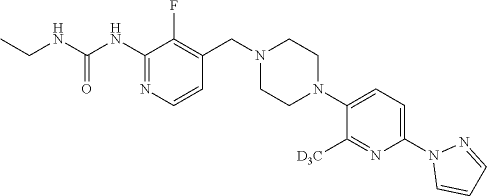 [SMILES: [2H]C([2H])([2H])c1nc(-n2cccn2)ccc1N1CCN(Cc2ccnc(NC(=O)NCC)c2F)CC1] | 1-ethyl-3-(3-fluoro-4-((4-(2-(methyl-d3)- 6-(1H-pyrazol-1-yl)pyridin-3- yl)piperazin-1-yl)methyl)pyridin-2- yl)urea |
| 467 | 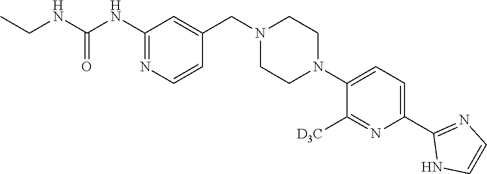 [SMILES: [2H]C([2H])([2H])c1nc(-c2ncc[nH]2)ccc1N1CCN(Cc2ccnc(NC(=O)NCC)c2)CC1] | 1-(4-((4-(6-(1H-imidazol-2-yl)-2-(methyl- d3)pyridin-3-yl)piperazin-1- yl)methyl)pyridin-2-yl)-3-ethylurea |
| 468 | 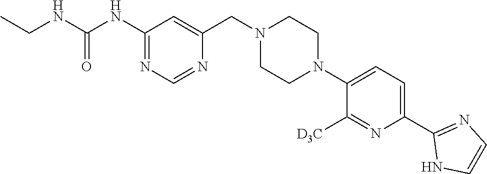 [SMILES: [2H]C([2H])([2H])c1nc(-c2ncc[nH]2)ccc1N1CCN(Cc2cc(NC(=O)NCC)ncn2)CC1] | 1-(6-((4-(6-(1H-imidazol-2-yl)-2-(methyl- d3)pyridin-3-yl)piperazin-1- yl)methyl)pyrimidin-4-yl)-3-ethylurea |
| 469 | 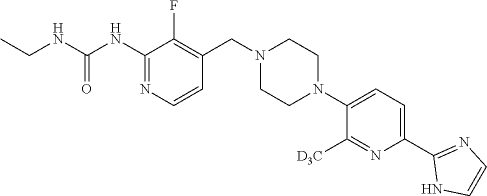 [SMILES: [2H]C([2H])([2H])c1nc(-c2ncc[nH]2)ccc1N1CCN(Cc2ccnc(NC(=O)NCC)c2F)CC1] | 1-(4-((4-(6-(1H-imidazol-2-yl)-2-(methyl- d3)pyridin-3-yl)piperazin-1-yl)methyl)-3- fluoropyridin-2-yl)-3-ethylurea |
| 470 | 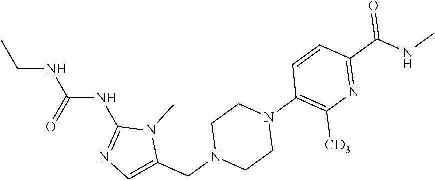 [SMILES: [2H]C([2H])([2H])c1nc(C(=O)NC)ccc1N1CCN(Cc2cnc(NC(=O)NCC)n2C)CC1] | 5-(4-((2-(3-ethylureido)-1-methyl-1H- imidazol-5-yl)methyl)piperazin-1-yl)-N- methyl-6-(methyl-d3)picolinamide |
| 471 | 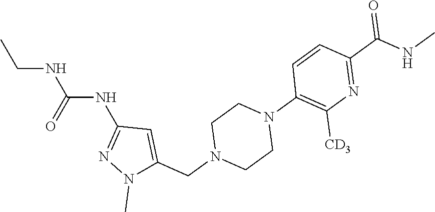 [SMILES: [2H]C([2H])([2H])c1nc(C(=O)NC)ccc1N1CCN(Cc2cc(NC(=O)NCC)nn2C)CC1] | 5-(4-((3-(3-ethylureido)-1-methyl-1H- pyrazol-5-yl)methyl)piperazin-1-yl)-N- methyl-6-(methyl-d3)picolinamide |
| 472 | 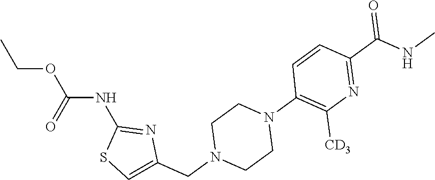 [SMILES: [2H]C([2H])([2H])c1nc(C(=O)NC)ccc1N1CCN(Cc2csc(NC(=O)OCC)n2)CC1] | ethyl (4-((4-(2-(methyl-d3)-6- (methylcarbamoyl)pyridin-3-yl)piperazin- 1-yl)methyl)thiazol-2-yl)carbamate |
| 473 | 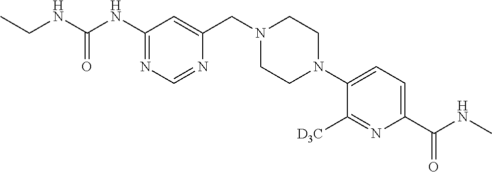 [SMILES: [2H]C([2H])([2H])c1nc(C(=O)NC)ccc1N1CCN(Cc2cc(NC(=O)NCC)ncn2)CC1] | 5-(4-((6-(3-ethylureido)pyrimidin-4- yl)methyl)piperazin-1-yl)-N-methyl-6- (methyl-d3)picolinamide |
| 474 | 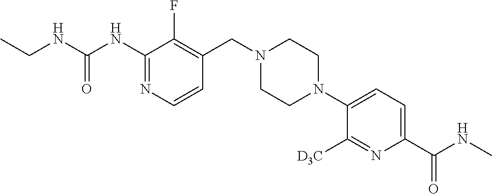 [SMILES: [2H]C([2H])([2H])c1nc(C(=O)NC)ccc1N1CCN(Cc2ccnc(NC(=O)NCC)c2F)CC1] | 5-(4-((2-(3-ethylureido)-3-fluoropyridin- 4-yl)methyl)piperazin-1-yl)-N-methyl-6- (methyl-d3)picolinamide |
| 475 | 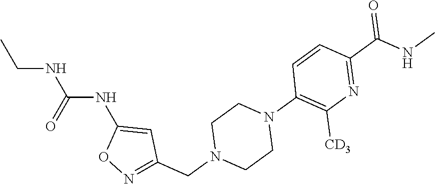 [SMILES: [2H]C([2H])([2H])c1nc(C(=O)NC)ccc1N1CCN(Cc2cc(NC(=O)NCC)on2)CC1] | 5-(4-((5-(3-ethylureido)isoxazol-3- yl)methyl)piperazin-1-yl)-N-methyl-6- (methyl-d3)picolinamide |
| 476 | 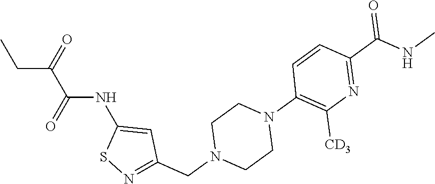 [SMILES: [2H]C([2H])([2H])c1nc(C(=O)NC)ccc1N1CCN(Cc2cc(NC(=O)C(=O)CC)sn2)CC1] | N-methyl-6-(methyl-d3)-5-(4-((5-(2- oxobutanamido)isothiazol-3- yl)methyl)piperazin-1-yl)picolinamide |
| 477 | 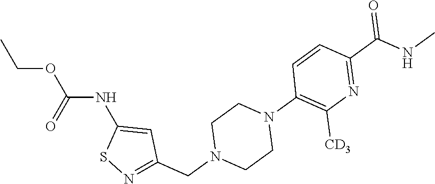 [SMILES: [2H]C([2H])([2H])c1nc(C(=O)NC)ccc1N1CCN(Cc2cc(NC(=O)OCC)sn2)CC1] | ethyl (3-((4-(2-(methyl-d3)-6- (methylcarbamoyl)pyridin-3-yl)piperazin- 1-yl)methyl)isothiazol-5-yl)carbamate |
| 478 | 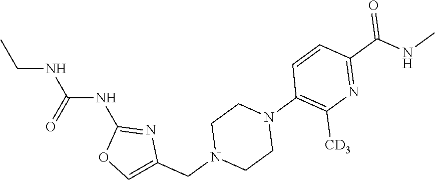 [SMILES: [2H]C([2H])([2H])c1nc(C(=O)NC)ccc1N1CCN(Cc2coc(NC(=O)NCC)n2)CC1] | 5-(4-((2-(3-ethylureido)oxazol-4- yl)methyl)piperazin-1-yl)-N-methyl-6- (methyl-d3)picolinamide |
| 479 | 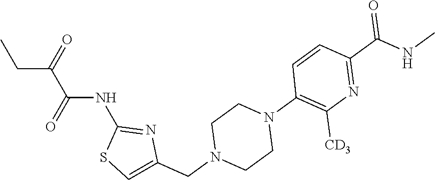 [SMILES: [2H]C([2H])([2H])c1nc(C(=O)NC)ccc1N1CCN(Cc2csc(NC(=O)C(=O)CC)n2)CC1] | N-methyl-6-(methyl-d3)-5-(4-((2-(2- oxobutanamido)thiazol-4- yl)methyl)piperazin-1-yl)picolinamide |
| 480 | 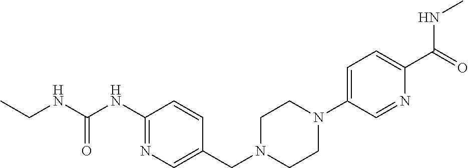 [SMILES: CCNC(=O)Nc1ccc(CN2CCN(c3ccc(C(=O)NC)nc3)CC2)cn1] | 5-(4-((6-(3-ethylureido)pyridin-3- yl)methyl)piperazin-1-yl)-N- methylpicolinamide |
| 481 | 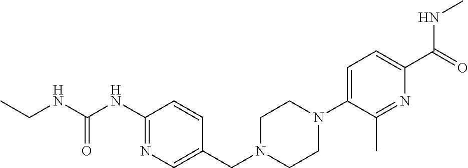 [SMILES: CCNC(=O)Nc1ccc(CN2CCN(c3ccc(C(=O)NC)nc3C)CC2)cn1] | 5-(4-((6-(3-ethylureido)pyridin-3- yl)methyl)piperazin-1-yl)-N,6- dimethylpicolinamide |
| 482 | 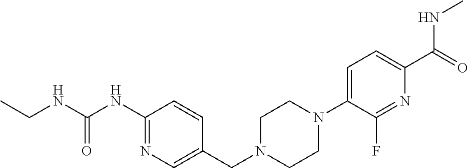 [SMILES: CCNC(=O)Nc1ccc(CN2CCN(c3ccc(C(=O)NC)nc3F)CC2)cn1] | 5-(4-((6-(3-ethylureido)pyridin-3- yl)methyl)piperazin-1-yl)-6-fluoro-N- methylpicolinamide |
| 483 | 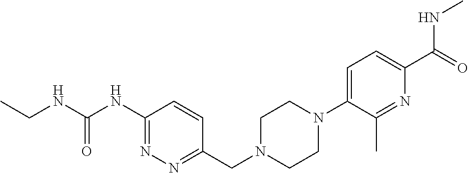 [SMILES: CCNC(=O)Nc1ccc(CN2CCN(c3ccc(C(=O)NC)nc3C)CC2)nn1] | 5-(4-((6-(3-ethylureido)pyridazin-3- yl)methyl)piperazin-1-yl)-N,6- dimethylpicolinamide |
| 484 | 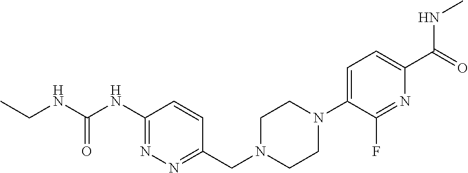 [SMILES: CCNC(=O)Nc1ccc(CN2CCN(c3ccc(C(=O)NC)nc3F)CC2)nn1] | 5-(4-((6-(3-ethylureido)pyridazin-3- yl)methyl)piperazin-1-yl)-6-fluoro-N- methylpicolinamide |
| 485 | 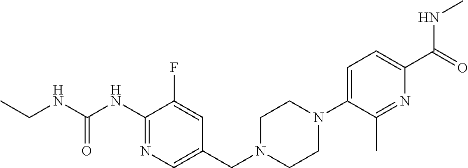 [SMILES: CCNC(=O)Nc1ncc(CN2CCN(c3ccc(C(=O)NC)nc3C)CC2)cc1F] | 5-(4-((6-(3-ethylureido)-5-fluoropyridin- 3-yl)methyl)piperazin-1-yl)-N,6- dimethylpicolinamide |
| 486 | 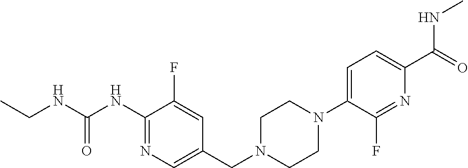 [SMILES: CCNC(=O)Nc1ncc(CN2CCN(c3ccc(C(=O)NC)nc3F)CC2)cc1F] | 5-(4-((6-(3-ethylureido)-5-fluoropyridin- 3-yl)methyl)piperazin-1-yl)-6-fluoro-N- methylpicolinamide |
| 487 | 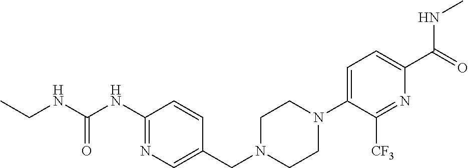 [SMILES: CCNC(=O)Nc1ccc(CN2CCN(c3ccc(C(=O)NC)nc3C(F)(F)F)CC2)cn1] | 5-(4-((6-(3-ethylureido)pyridin-3- yl)methyl)piperazin-1-yl)-N-methyl-6- (trifluoromethyl)picolinamide |
| 488 | 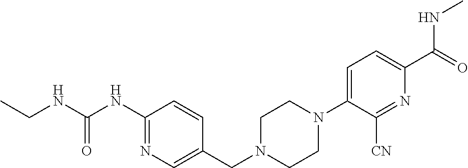 [SMILES: CCNC(=O)Nc1ccc(CN2CCN(c3ccc(C(=O)NC)nc3C#N)CC2)cn1] | 6-cyano-5-(4-((6-(3-ethylureido)pyridin- 3-yl)methyl)piperazin-1-yl)-N- methylpicolinamide |
| 489 | 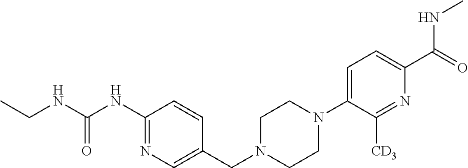 [SMILES: [2H]C([2H])([2H])c1nc(C(=O)NC)ccc1N1CCN(Cc2ccc(NC(=O)NCC)nc2)CC1] | 5-(4-((6-(3-ethylureido)pyridin-3- yl)methyl)piperazin-1-yl)-N-methyl-6- (methyl-d3)picolinamide |
| 490 | 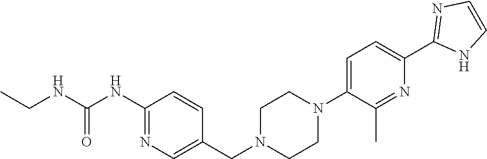 [SMILES: CCNC(=O)Nc1ccc(CN2CCN(c3ccc(-c4ncc[nH]4)nc3C)CC2)cn1] | 1-(5-((4-(6-(1H-imidazol-2-yl)-2- methylpyridin-3-yl)piperazin-1- yl)methyl)pyridin-2-yl)-3-ethylurea |
| 491 | 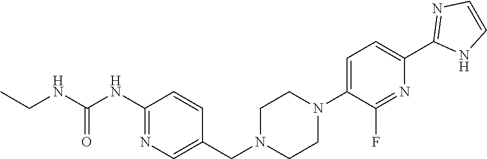 [SMILES: CCNC(=O)Nc1ccc(CN2CCN(c3ccc(-c4ncc[nH]4)nc3F)CC2)cn1] | 1-ethyl-3-(5-((4-(2-fluoro-6-(1H- imidazol-2-yl)pyridin-3-yl)piperazin-1- yl)methyl)pyridin-2-yl)urea |
| 492 | 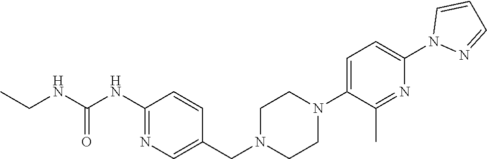 [SMILES: CCNC(=O)Nc1ccc(CN2CCN(c3ccc(-n4cccn4)nc3C)CC2)cn1] | 1-ethyl-3-(5-((4-(2-methyl-6-(1H- pyrazol-1-yl)pyridin-3-yl)piperazin-1- yl)methyl)pyridin-2-yl)urea |
| 493 | 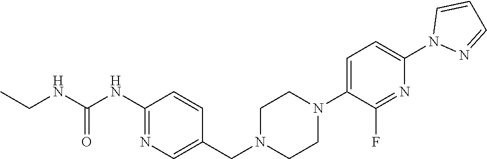 [SMILES: CCNC(=O)Nc1ccc(CN2CCN(c3ccc(-n4cccn4)nc3F)CC2)cn1] | 1-ethyl-3-(5-((4-(2-fluoro-6-(1H-pyrazol- 1-yl)pyridin-3-yl)piperazin-1- yl)methyl)pyridin-2-yl)urea |
| 494 | 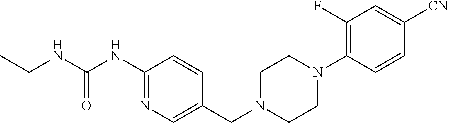 [SMILES: CCNC(=O)Nc1ccc(CN2CCN(c3ccc(C#N)cc3F)CC2)cn1] | 1-(5-((4-(4-cyano-2- fluorophenyl)piperazin-1- yl)methyl)pyridin-2-yl)-3-ethylurea |
| 495 | 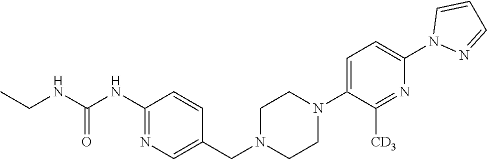 [SMILES: [2H]C([2H])([2H])c1nc(-n2cccn2)ccc1N1CCN(Cc2ccc(NC(=O)NCC)nc2)CC1] | 1-ethyl-3-(5-((4-(2-(methyl-d3)-6-(1H- pyrazol-1-yl)pyridin-3-yl)piperazin-1- yl)methyl)pyridin-2-yl)urea |
| 496 | 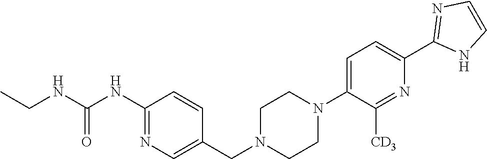 [SMILES: [2H]C([2H])([2H])c1nc(-c2ncc[nH]2)ccc1N1CCN(Cc2ccc(NC(=O)NCC)nc2)CC1] | 1-(5-((4-(6-(1H-imidazol-2-yl)-2-(methyl- d3)pyridin-3-yl)piperazin-1- yl)methyl)pyridin-2-yl)-3-ethylurea |
| 497 | 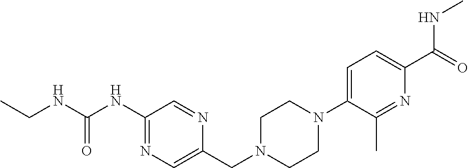 [SMILES: CCNC(=O)Nc1cnc(CN2CCN(c3ccc(C(=O)NC)nc3C)CC2)cn1] | 5-(4-((5-(3-ethylureido)pyrazin-2- yl)methyl)piperazin-1-yl)-N,6- dimethylpicolinamide |
| 498 | 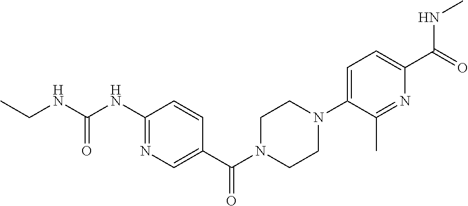 [SMILES: CCNC(=O)Nc1ccc(C(=O)N2CCN(c3ccc(C(=O)NC)nc3C)CC2)cn1] | 5-(4-(6-(3- ethylureido)nicotinoyl)piperazin-1-yl)- N,6-dimethylpicolinamide |
| 499 | 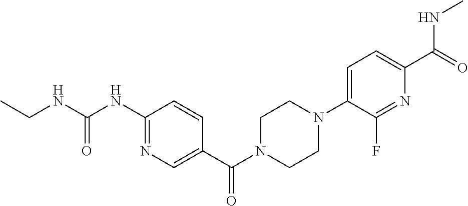 [SMILES: CCNC(=O)Nc1ccc(C(=O)N2CCN(c3ccc(C(=O)NC)nc3F)CC2)cn1] | 5-(4-(6-(3- ethylureido)nicotinoyl)piperazin-1-yl)-6- fluoro-N-methylpicolinamide |
| 500 | 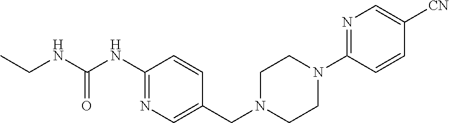 [SMILES: CCNC(=O)Nc1ccc(CN2CCN(c3ccc(C#N)cn3)CC2)cn1] | 1-(5-((4-(5-cyanopyridin-2-yl)piperazin- 1-yl)methyl)pyridin-2-yl)-3-ethylurea |
| 501 | 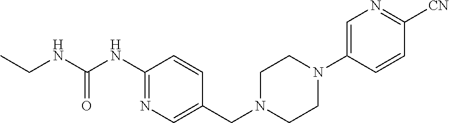 [SMILES: CCNC(=O)Nc1ccc(CN2CCN(c3ccc(C#N)nc3)CC2)cn1] | 1-(5-((4-(6-cyanopyridin-3-yl)piperazin- 1-yl)methyl)pyridin-2-yl)-3-ethylurea |
| 502 | 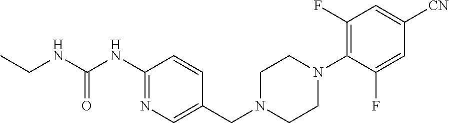 [SMILES: CCNC(=O)Nc1ccc(CN2CCN(c3c(F)cc(C#N)cc3F)CC2)cn1] | 1-(5-((4-(4-cyano-2,6- difluorophenyl)piperazin-1- yl)methyl)pyridin-2-yl)-3-ethylurea |
| 503 | 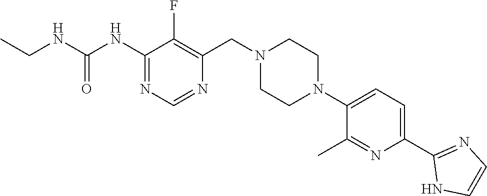 [SMILES: CCNC(=O)Nc1ncnc(CN2CCN(c3ccc(-c4ncc[nH]4)nc3C)CC2)c1F] | 1-(6-((4-(6-(1H-imidazol-2-yl)-2- methylpyridin-3-yl)piperazin-1- yl)methyl)-5-fluoropyrimidin-4-yl)-3- ethylurea |
| 504 | 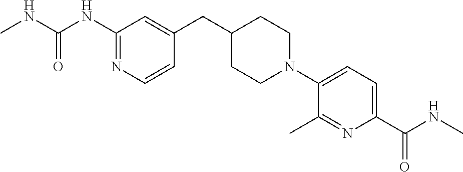 | N,6-dimethyl-5-(4-((2-(3- methylureido)pyridin-4- yl)methyl)piperidin-1-yl)picolinamide |
| 505 | 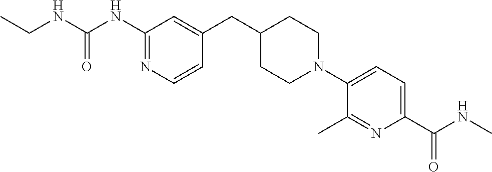 [SMILES: CCNC(=O)Nc1cc(CC2CCN(c3ccc(C(=O)NC)nc3C)CC2)ccn1] | 5-(4-((2-(3-ethylureido)pyridin-4- yl)methyl)piperidin-1-yl)-N,6- dimethylpicolinamide |
| 506 | 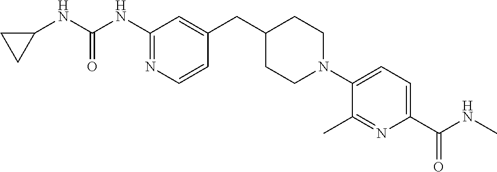 [SMILES: CNC(=O)c1ccc(N2CCC(Cc3ccnc(NC(=O)NC4CC4)c3)CC2)c(C)n1] | 5-(4-((2-(3-cyclopropylureido)pyridin-4- yl)methyl)piperidin-1-yl)-N,6- dimethylpicolinamide |
| 507 | 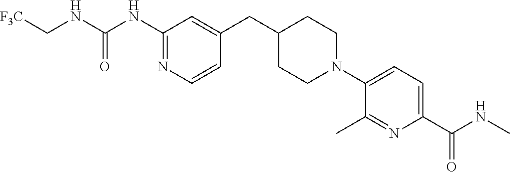 [SMILES: CNC(=O)c1ccc(N2CCC(Cc3ccnc(NC(=O)NCC(F)(F)F)c3)CC2)c(C)n1] | N,6-dimethyl-5-(4-((2-(3-(2,2,2- trifluoroethyl)ureido)pyridin-4- yl)methyl)piperidin-1-yl)picolinamide |
| 508 | 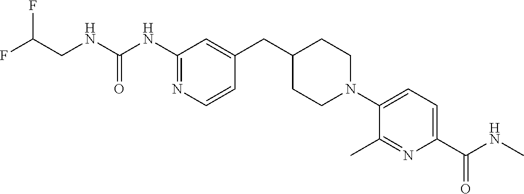 [SMILES: CNC(=O)c1ccc(N2CCC(Cc3ccnc(NC(=O)NCC(F)F)c3)CC2)c(C)n1] | 5-(4-((2-(3-(2,2- difluoroethyl)ureido)pyridin-4- yl)methyl)piperidin-1-yl)-N,6- dimethylpicolinamide |
| 509 | 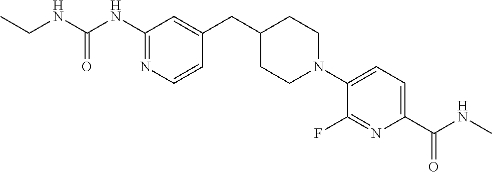 [SMILES: CCNC(=O)Nc1cc(CC2CCN(c3ccc(C(=O)NC)nc3F)CC2)ccn1] | 5-(4-((2-(3-ethylureido)pyridin-4- yl)methyl)piperidin-1-yl)-6-fluoro-N- methylpicolinamide |
| 510 | 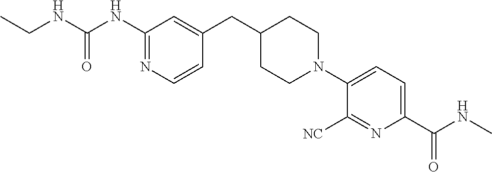 [SMILES: CCNC(=O)Nc1cc(CC2CCN(c3ccc(C(=O)NC)nc3C#N)CC2)ccn1] | 6-cyano-5-(4-((2-(3-ethylureido)pyridin- 4-yl)methyl)piperidin-1-yl)-N- methylpicolinamide |
| 511 | 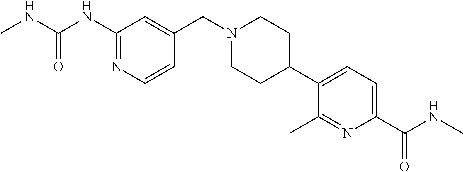 | N,6-dimethyl-5-(1-((2-(3- methylureido)pyridin-4- yl)methyl)piperidin-4-yl)picolinamide |
| 512 | 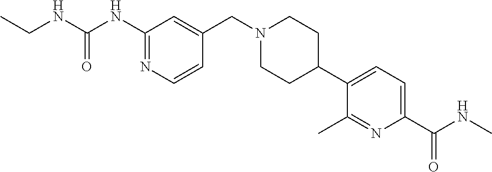 [SMILES: CCNC(=O)Nc1cc(CN2CCC(c3ccc(C(=O)NC)nc3C)CC2)ccn1] | 5-(1-((2-(3-ethylureido)pyridin-4- yl)methyl)piperidin-4-yl)-N,6- dimethylpicolinamide |
| 513 | 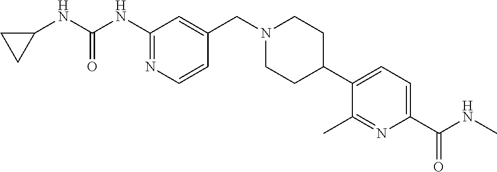 [SMILES: CNC(=O)c1ccc(C2CCN(Cc3ccnc(NC(=O)NC4CC4)c3)CC2)c(C)n1] | 5-(1-((2-(3-cyclopropylureido)pyridin-4- yl)methyl)piperidin-4-yl)-N,6- dimethylpicolinamide |
| 514 | 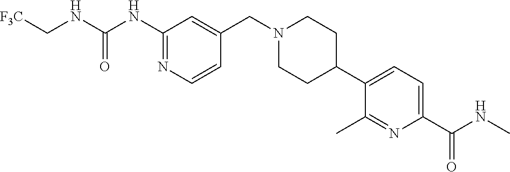 [SMILES: CNC(=O)c1ccc(C2CCN(Cc3ccnc(NC(=O)NCC(F)(F)F)c3)CC2)c(C)n1] | N,6-dimethyl-5-(1-((2-(3-(2,2,2- trifluoroethyl)ureido)pyridin-4- yl)methyl)piperidin-4-yl)picolinamide |
| 515 | 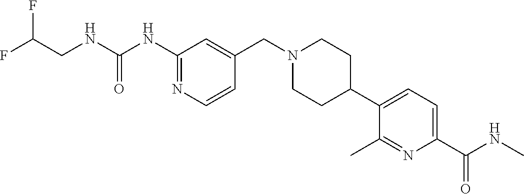 [SMILES: CNC(=O)c1ccc(C2CCN(Cc3ccnc(NC(=O)NCC(F)F)c3)CC2)c(C)n1] | 5-(1-((2-(3-(2,2- difluoroethyl )ureido)pyridin-4- yl)methyl)piperidin-4-yl)-N,6- dimethylpicolinamide |
| 516 | 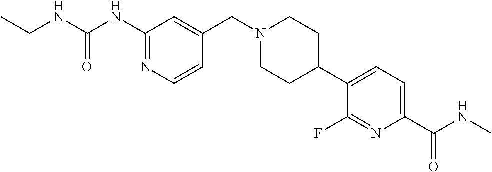 [SMILES: CCNC(=O)Nc1cc(CN2CCC(c3ccc(C(=O)NC)nc3F)CC2)ccn1] | 5-(1-((2-(3-ethylureido)pyridin-4- yl)methyl)piperidin-4-yl)-6-fluoro-N- methylpicolinamide |
| 517 | 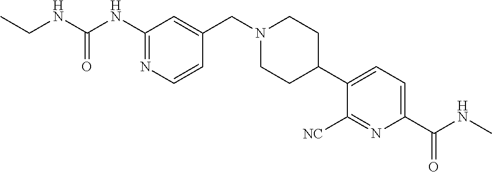 [SMILES: CCNC(=O)Nc1cc(CN2CCC(c3ccc(C(=O)NC)nc3C#N)CC2)ccn1] | 6-cyano-5-(1-((2-(3-ethylureido)pyridin- 4-yl)methyl)piperidin-4-yl)-N- methylpicolinamide |
| 518 | 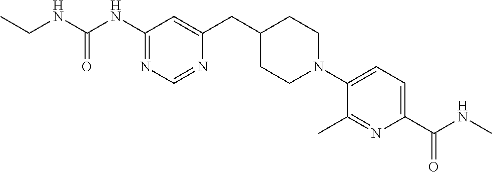 [SMILES: CCNC(=O)Nc1cc(CC2CCN(c3ccc(C(=O)NC)nc3C)CC2)ncn1] | 5-(4-((6-(3-ethylureido)pyrimidin-4- yl)methyl)piperidin-1-yl)-N,6- dimethylpicolinamide |
| 519 | 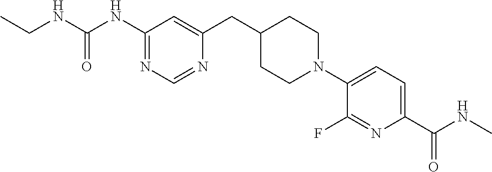 [SMILES: CCNC(=O)Nc1cc(CC2CCN(c3ccc(C(=O)NC)nc3F)CC2)ncn1] | 5-(4-((6-(3-ethylureido)pyrimidin-4- yl)methyl)piperidin-1-yl)-6-fluoro-N- methylpicolinamide |
| 520 | 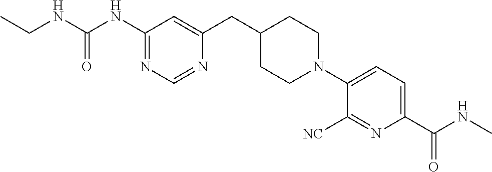 [SMILES: CCNC(=O)Nc1cc(CC2CCN(c3ccc(C(=O)NC)nc3C#N)CC2)ncn1] | 6-cyano-5-(4-((6-(3- ethylureido)pyrimidin-4- yl)methyl)piperidin-1-yl)-N- methylpicolinamide |
| 521 | 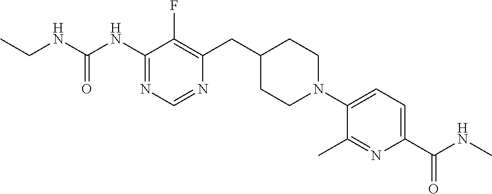 [SMILES: CCNC(=O)Nc1ncnc(CC2CCN(c3ccc(C(=O)NC)nc3C)CC2)c1F] | 5-(4-((6-(3-ethylureido)-5- fluoropyrimidin-4-yl)methyl)piperidin-1- yl)-N,6-dimethylpicolinamide |
| 522 | 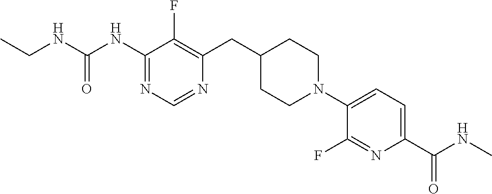 [SMILES: CCNC(=O)Nc1ncnc(CC2CCN(c3ccc(C(=O)NC)nc3F)CC2)c1F] | 5-(4-((6-(3-ethylureido)-5- fluoropyrimidin-4-yl)methyl)piperidin-1- yl)-6-fluoro-N-methylpicolinamide |
| 523 | 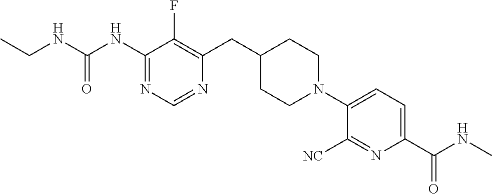 [SMILES: CCNC(=O)Nc1ncnc(CC2CCN(c3ccc(C(=O)NC)nc3C#N)CC2)c1F] | 6-cyano-5-(4-((6-(3-ethylureido)-5- fluoropyrimidin-4-yl)methyl)piperidin-1- yl)-N-methylpicolinamide |
| 524 | 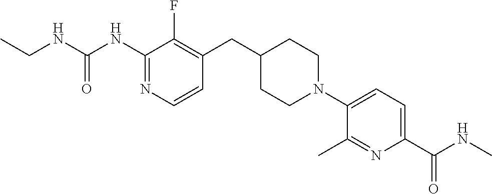 [SMILES: CCNC(=O)Nc1nccc(CC2CCN(c3ccc(C(=O)NC)nc3C)CC2)c1F] | 5-(4-((2-(3-ethylureido)-3-fluoropyridin- 4-yl)methyl)piperidin-1-yl)-N,6- dimethylpicolinamide |
| 525 | 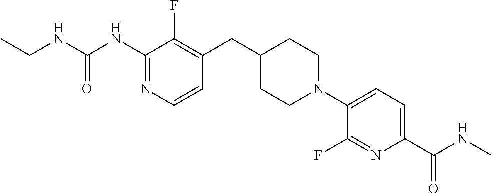 [SMILES: CCNC(=O)Nc1nccc(CC2CCN(c3ccc(C(=O)NC)nc3F)CC2)c1F] | 5-(4-((2-(3-ethylureido)-3-fluoropyridin- 4-yl)methyl)piperidin-1-yl)-6-fluoro-N- methylpicolinamide |
| 526 | 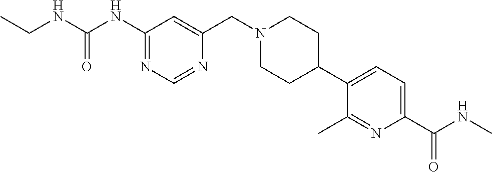 [SMILES: CCNC(=O)Nc1cc(CN2CCC(c3ccc(C(=O)NC)nc3C)CC2)ncn1] | 5-(1-((6-(3-ethylureido)pyrimidin-4- yl)methyl)piperidin-4-yl)-N,6- dimethylpicolinamide |
| 527 | 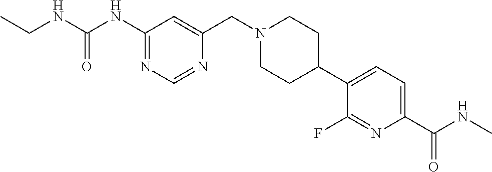 [SMILES: CCNC(=O)Nc1cc(CN2CCC(c3ccc(C(=O)NC)nc3F)CC2)ncn1] | 5-(1-((6-(3-ethylureido)pyrimidin-4- yl)methyl)piperidin-4-yl)-6-fluoro-N- methylpicolinamide |
| 528 | 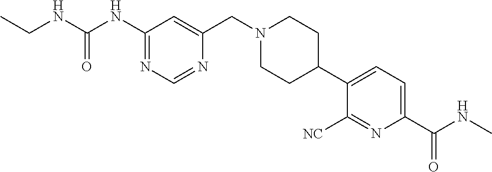 [SMILES: CCNC(=O)Nc1cc(CN2CCC(c3ccc(C(=O)NC)nc3C#N)CC2)ncn1] | 6-cyano-5-(1-((6-(3- ethylureido)pyrimidin-4- yl)methyl)piperidin-4-yl)-N- methylpicolinamide |
| 529 | 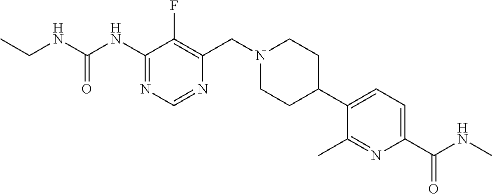 [SMILES: CCNC(=O)Nc1ncnc(CN2CCC(c3ccc(C(=O)NC)nc3C)CC2)c1F] | 5-(1-((6-(3-ethylureido)-5- fluoropyrimidin-4-yl)methyl)piperidin-4- yl)-N,6-dimethylpicolinamide |
| 530 | 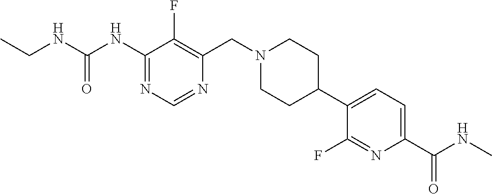 [SMILES: CCNC(=O)Nc1ncnc(CN2CCC(c3ccc(C(=O)NC)nc3F)CC2)c1F] | 5-(1-((6-(3-ethylureido)-5- fluoropyrimidin-4-yl)methyl)piperidin-4- yl)-6-fluoro-N-methylpicolinamide |
| 531 | 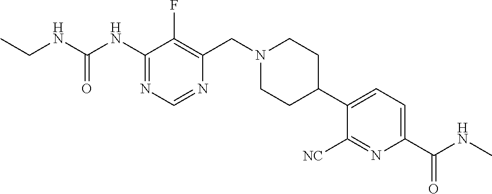 [SMILES: CCNC(=O)Nc1ncnc(CN2CCC(c3ccc(C(=O)NC)nc3C#N)CC2)c1F] | 6-cyano-5-(1-((6-(3-ethylureido)-5- fluoropyrimidin-4-yl)methyl)piperidin-4- yl)-N-methylpicolinamide |
| 532 | 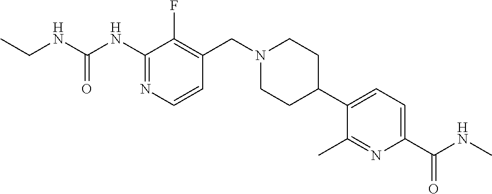 [SMILES: CCNC(=O)Nc1nccc(CN2CCC(c3ccc(C(=O)NC)nc3C)CC2)c1F] | 5-(1-((2-(3-ethylureido)-3-fluoropyridin- 4-yl)methyl)piperidin-4-yl)-N,6- dimethylpicolinamide |
| 533 | 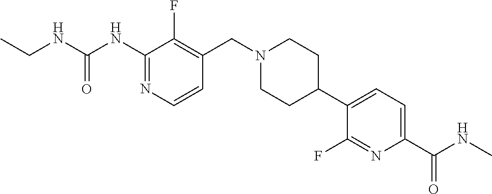 [SMILES: CCNC(=O)Nc1nccc(CN2CCC(c3ccc(C(=O)NC)nc3F)CC2)c1F] | 5-(1-((2-(3-ethylureido)-3-fluoropyridin- 4-yl)methyl)piperidin-4-yl)-6-fluoro-N- methylpicolinamide |
| 534 | 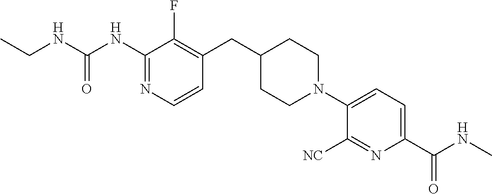 [SMILES: CCNC(=O)Nc1nccc(CC2CCN(c3ccc(C(=O)NC)nc3C#N)CC2)c1F] | 6-cyano-5-(4-((2-(3-ethylureido)-3- fluoropyridin-4-yl)methyl)piperidin-1-yl)- N-methylpicolinamide |
| 535 | 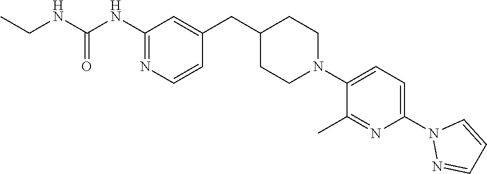 [SMILES: CCNC(=O)Nc1cc(CC2CCN(c3ccc(-n4cccn4)nc3C)CC2)ccn1] | 1-ethyl-3-(4-((1-(2-methyl-6-(1H- pyrazol-1-yl)pyridin-3-yl)piperidin-4- yl)methyl)pyridin-2-yl)urea |
| 536 | 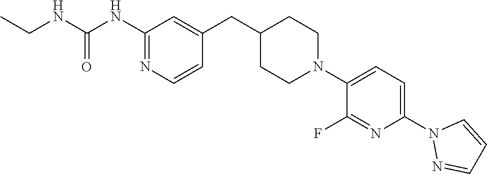 [SMILES: CCNC(=O)Nc1cc(CC2CCN(c3ccc(-n4cccn4)nc3F)CC2)ccn1] | 1-ethyl-3-(4-((1-(2-fluoro-6-(1H-pyrazol- 1-yl)pyridin-3-yl)piperidin-4- yl)methyl)pyridin-2-yl)urea |
| 537 | 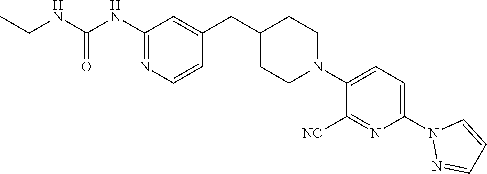 [SMILES: CCNC(=O)Nc1cc(CC2CCN(c3ccc(-n4cccn4)nc3C#N)CC2)ccn1] | 1-(4-((1-(2-cyano-6-(1H-pyrazol-1- yl)pyridin-3-yl)piperidin-4- yl)methyl)pyridin-2-yl)-3-ethylurea |
| 538 | 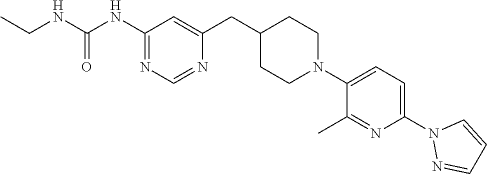 [SMILES: CCNC(=O)Nc1cc(CC2CCN(c3ccc(-n4cccn4)nc3C)CC2)ncn1] | 1-ethyl-3-(6-((1-(2-methyl-6-(1H- pyrazol-1-yl)pyridin-3-yl)piperidin-4- yl)methyl)pyrimidin-4-yl)urea |
| 539 | 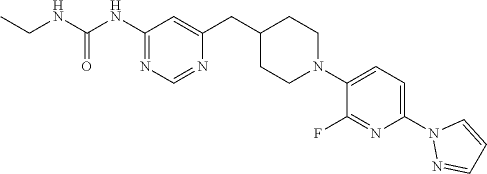 [SMILES: CCNC(=O)Nc1cc(CC2CCN(c3ccc(-n4cccn4)nc3F)CC2)ncn1] | 1-ethyl-3-(6-((1-(2-fluoro-6-(1H-pyrazol- 1-yl)pyridin-3-yl)piperidin-4- yl)methyl)pyrimidin-4-yl)urea |
| 540 | 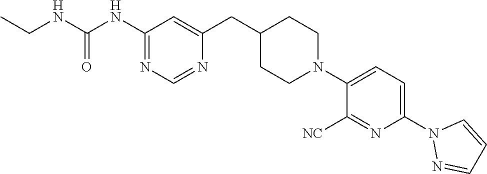 [SMILES: CCNC(=O)Nc1cc(CC2CCN(c3ccc(-n4cccn4)nc3C#N)CC2)ncn1] | 1-(6-((1-(2-cyano-6-(1H-pyrazol-1- yl)pyridin-3-yl)piperidin-4- yl)methyl)pyrimidin-4-yl)-3-ethylurea |
| 541 | 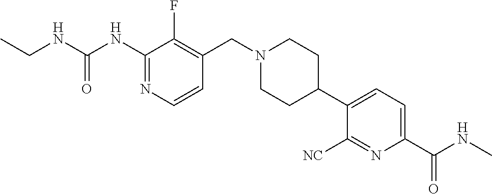 [SMILES: CCNC(=O)Nc1nccc(CN2CCC(c3ccc(C(=O)NC)nc3C#N)CC2)c1F] | 6-cyano-5-(1-((2-(3-ethylureido)-3- fluoropyridin-4-yl)methyl)piperidin-4-yl)- N-methylpicolinamide |
| 542 | 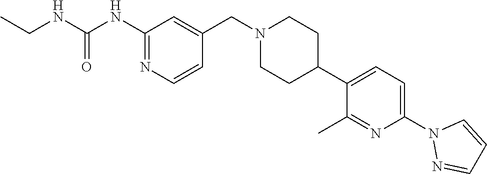 [SMILES: CCNC(=O)Nc1cc(CN2CCC(c3ccc(-n4cccn4)nc3C)CC2)ccn1] | 1-ethyl-3-(4-((4-(2-methyl-6-(1H- pyrazol-1-yl)pyridin-3-yl)piperidin-1- yl)methyl)pyridin-2-yl)urea |
| 543 | 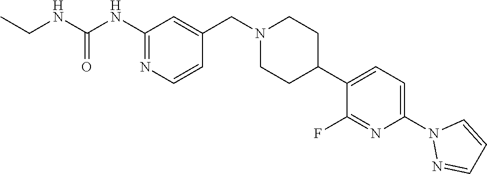 [SMILES: CCNC(=O)Nc1cc(CN2CCC(c3ccc(-n4cccn4)nc3F)CC2)ccn1] | 1-ethyl-3-(4-((4-(2-fluoro-6-(1H-pyrazol- 1-yl)pyridin-3-yl)piperidin-1- yl)methyl)pyridin-2-yl)urea |
| 544 | 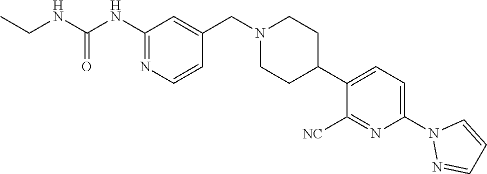 [SMILES: CCNC(=O)Nc1cc(CN2CCC(c3ccc(-n4cccn4)nc3C#N)CC2)ccn1] | 1-(4-((4-(2-cyano-6-(1H-pyrazol-1- yl)pyridin-3-yl)piperidin-1- yl)methyl)pyridin-2-yl)-3-ethylurea |
| 545 | 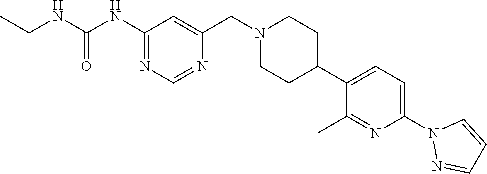 [SMILES: CCNC(=O)Nc1cc(CN2CCC(c3ccc(-n4cccn4)nc3C)CC2)ncn1] | 1-ethyl-3-(6-((4-(2-methyl-6-(1H- pyrazol-1-yl)pyridin-3-yl)piperidin-1- yl)methyl)pyrimidin-4-yl)urea |
| 546 | 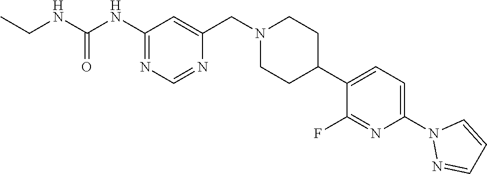 [SMILES: CCNC(=O)Nc1cc(CN2CCC(c3ccc(-n4cccn4)nc3F)CC2)ncn1] | 1-ethyl-3-(6-((4-(2-fluoro-6-(1H-pyrazol- 1-yl)pyridin-3-yl)piperidin-1- yl)methyl)pyrimidin-4-yl)urea |
| 547 | 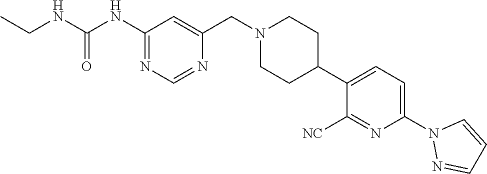 [SMILES: CCNC(=O)Nc1cc(CN2CCC(c3ccc(-n4cccn4)nc3C#N)CC2)ncn1] | 1-(6-((4-(2-cyano-6-(1H-pyrazol-1- yl)pyridin-3-yl)piperidin-1- yl)methyl)pyrimidin-4-yl)-3-ethylurea |
| 548 | 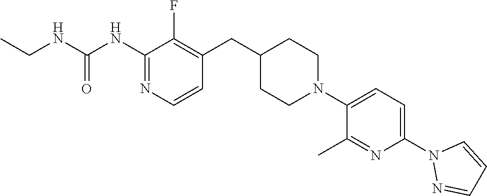 [SMILES: CCNC(=O)Nc1nccc(CC2CCN(c3ccc(-n4cccn4)nc3C)CC2)c1F] | 1-ethyl-3-(3-fluoro-4-((1-(2-methyl-6- (1H-pyrazol-1-yl)pyridin-3-yl)piperidin- 4-yl)methyl)pyridin-2-yl)urea |
| 549 | 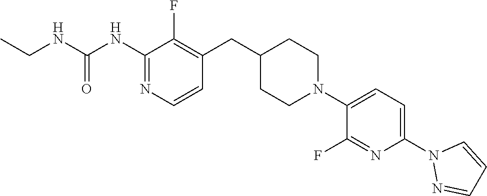 [SMILES: CCNC(=O)Nc1nccc(CC2CCN(c3ccc(-n4cccn4)nc3F)CC2)c1F] | 1-ethyl-3-(3-fluoro-4-((1-(2-fluoro-6-(1H- pyrazol-1-yl)pyridin-3-yl)piperidin-4- yl)methyl)pyridin-2-yl)urea |
| 550 | 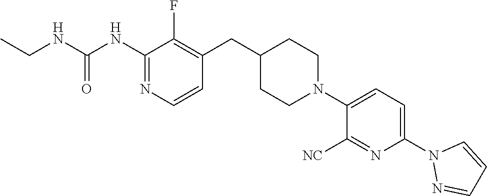 [SMILES: CCNC(=O)Nc1nccc(CC2CCN(c3ccc(-n4cccn4)nc3C#N)CC2)c1F] | 1-(4-((1-(2-cyano-6-(1H-pyrazol-1- yl)pyridin-3-yl)piperidin-4-yl)methyl)-3- fluoropyridin-2-yl)-3-ethylurea |
| 551 | 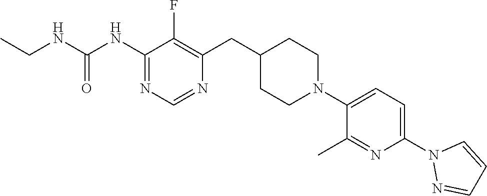 [SMILES: CCNC(=O)Nc1ncnc(CC2CCN(c3ccc(-n4cccn4)nc3C)CC2)c1F] | 1-ethyl-3-(5-fluoro-6-((1-(2-methyl-6- (1H-pyrazol-1-yl)pyridin-3-yl)piperidin- 4-yl)methyl)pyrimidin-4-yl)urea |
| 552 | 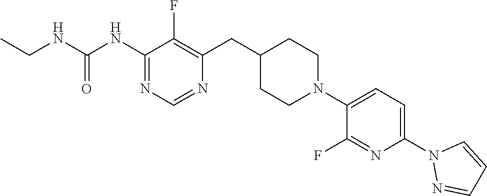 [SMILES: CCNC(=O)Nc1ncnc(CC2CCN(c3ccc(-n4cccn4)nc3F)CC2)c1F] | 1-ethyl-3-(5-fluoro-6-((1-(2-fluoro-6-(1H- pyrazol-1-yl)pyridin-3-yl)piperidin-4- yl)methyl)pyrimidin-4-yl)urea |
| 553 | 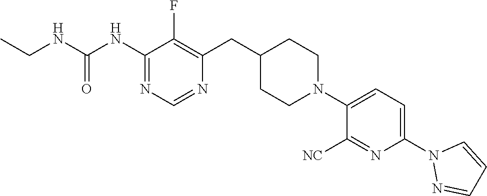 [SMILES: CCNC(=O)Nc1ncnc(CC2CCN(c3ccc(-n4cccn4)nc3C#N)CC2)c1F] | 1-(6-((1-(2-cyano-6-(1H-pyrazol-1- yl)pyridin-3-yl)piperidin-4-yl)methyl)-5- fluoropyrimidin-4-yl)-3-ethylurea |
| 554 | 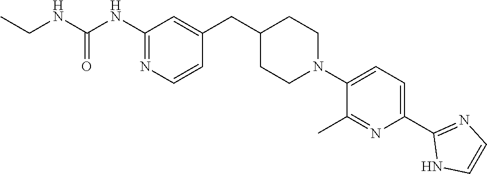 [SMILES: CCNC(=O)Nc1cc(CC2CCN(c3ccc(-c4ncc[nH]4)nc3C)CC2)ccn1] | 1-(4-((1-(6-(1H-imidazol-2-yl)-2- methylpyridin-3-yl)piperidin-4- yl)methyl)pyridin-2-yl)-3-ethylurea |
| 555 | 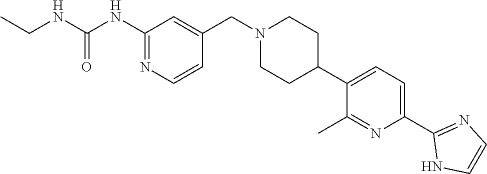 [SMILES: CCNC(=O)Nc1cc(CN2CCC(c3ccc(-c4ncc[nH]4)nc3C)CC2)ccn1] | 1-(4-((4-(6-(1H-imidazol-2-yl)-2- methylpyridin-3-yl)piperidin-1- yl)methyl)pyridin-2-yl)-3-ethylurea |
| 556 | 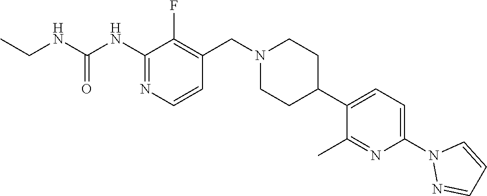 [SMILES: CCNC(=O)Nc1nccc(CN2CCC(c3ccc(-n4cccn4)nc3C)CC2)c1F] | 1-ethyl-3-(3-fluoro-4-((4-(2-methyl-6- (1H-pyrazol-1-yl)pyridin-3-yl)piperidin- 1-yl)methyl)pyridin-2-yl)urea |
| 557 | 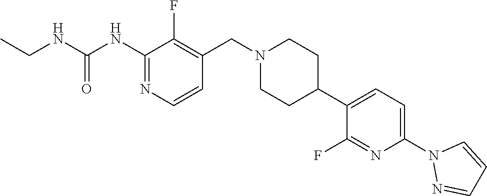 [SMILES: CCNC(=O)Nc1nccc(CN2CCC(c3ccc(-n4cccn4)nc3F)CC2)c1F] | 1-ethyl-3-(3-fluoro-4-((4-(2-fluoro-6-(1H- pyrazol-1-yl)pyridin-3-yl)piperidin-1- yl)methyl)pyridin-2-yl)urea |
| 558 | 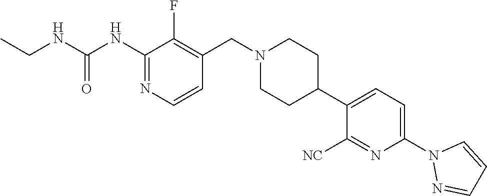 [SMILES: CCNC(=O)Nc1nccc(CN2CCC(c3ccc(-n4cccn4)nc3C#N)CC2)c1F] | 1-(4-((4-(2-cyano-6-(1H-pyrazol-1- yl)pyridin-3-yl)piperidin-1-yl)methyl)-3- fluoropyridin-2-yl)-3-ethylurea |
| 559 | 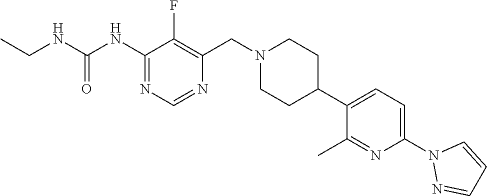 [SMILES: CCNC(=O)Nc1ncnc(CN2CCC(c3ccc(-n4cccn4)nc3C)CC2)c1F] | 1-ethyl-3-(5-fluoro-6-((4-(2-methyl-6- (1H-pyrazol-1-yl)pyridin-3-yl)piperidin- 1-yl)methyl)pyrimidin-4-yl)urea |
| 560 | 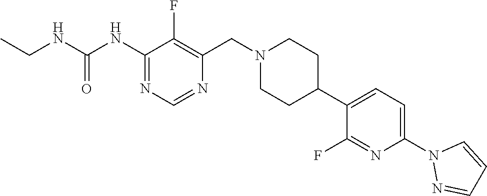 [SMILES: CCNC(=O)Nc1ncnc(CN2CCC(c3ccc(-n4cccn4)nc3F)CC2)c1F] | 1-ethyl-3-(5-fluoro-6-((4-(2-fluoro-6-(1H- pyrazol-1-yl)pyridin-3-yl)piperidin-1- yl)methyl)pyrimidin-4-yl)urea |
| 561 | 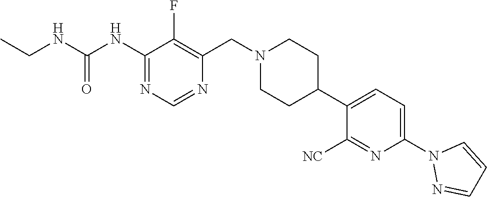 [SMILES: CCNC(=O)Nc1ncnc(CN2CCC(c3ccc(-n4cccn4)nc3C#N)CC2)c1F] | 1-(6-((4-(2-cyano-6-(1H-pyrazol-1- yl)pyridin-3-yl)piperidin-1-yl)methyl)-5- fluoropyrimidin-4-yl)-3-ethylurea |
| 562 | 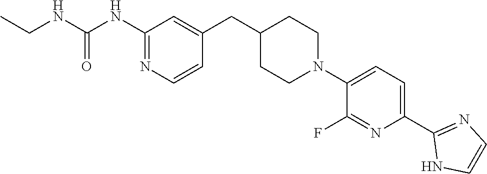 [SMILES: CCNC(=O)Nc1cc(CC2CCN(c3ccc(-c4ncc[nH]4)nc3F)CC2)ccn1] | 1-ethyl-3-(4-((1-(2-fluoro-6-(1H- imidazol-2-yl)pyridin-3-yl)piperidin-4- yl)methyl)pyridin-2-yl)urea |
| 563 | 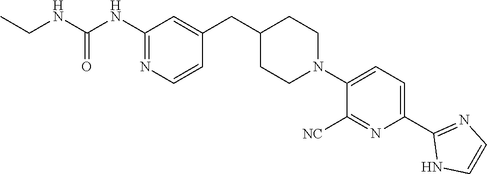 [SMILES: CCNC(=O)Nc1cc(CC2CCN(c3ccc(-c4ncc[nH]4)nc3C#N)CC2)ccn1] | 1-(4-((1-(2-cyano-6-(1H-imidazol-2- yl)pyridin-3-yl)piperidin-4- yl)methyl)pyridin-2-yl)-3-ethylurea |
| 564 | 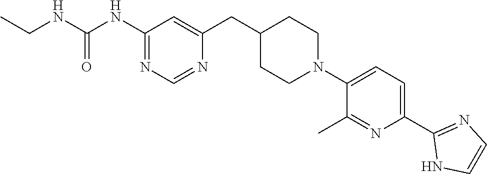 [SMILES: CCNC(=O)Nc1cc(CC2CCN(c3ccc(-c4ncc[nH]4)nc3C)CC2)ncn1] | 1-(6-((1-(6-(1H-imidazol-2-yl)-2- methylpyridin-3-yl)piperidin-4- yl)methyl)pyrimidin-4-yl)-3-ethylurea |
| 565 | 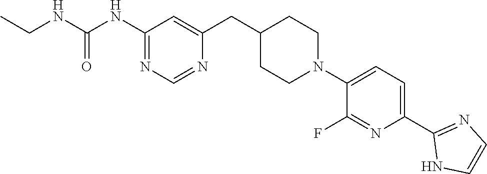 [SMILES: CCNC(=O)Nc1cc(CC2CCN(c3ccc(-c4ncc[nH]4)nc3F)CC2)ncn1] | 1-ethyl-3-(6-((1-(2-fluoro-6-(1H- imidazol-2-yl)pyridin-3-yl)piperidin-4- yl)methyl)pyrimidin-4-yl)urea |
| 566 | 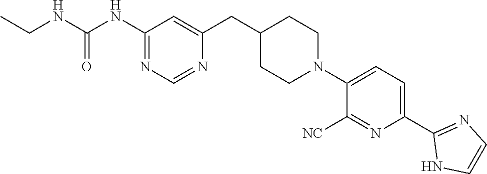 [SMILES: CCNC(=O)Nc1cc(CC2CCN(c3ccc(-c4ncc[nH]4)nc3C#N)CC2)ncn1] | 1-(6-((1-(2-cyano-6-(1H-imidazol-2- yl)pyridin-3-yl)piperidin-4- yl)methyl)pyrimidin-4-yl)-3-ethylurea |
| 567 | 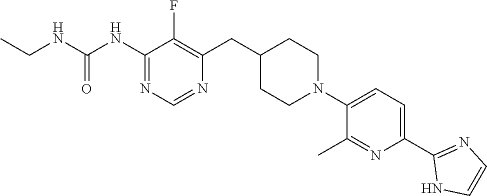 [SMILES: CCNC(=O)Nc1ncnc(CC2CCN(c3ccc(-c4ncc[nH]4)nc3C)CC2)c1F] | 1-(6-((1-(6-(1H-imidazol-2-yl)-2- methylpyridin-3-yl)piperidin-4- yl)methyl)-5-fluoropyrimidin-4-yl)-3- ethylurea |
| 568 | 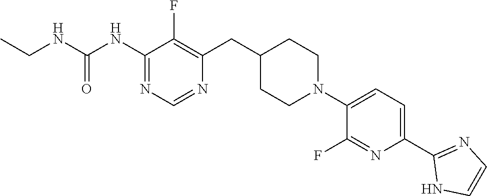 [SMILES: CCNC(=O)Nc1ncnc(CC2CCN(c3ccc(-c4ncc[nH]4)nc3F)CC2)c1F] | 1-ethyl-3-(5-fluoro-6-((1-(2-fluoro-6-(1H- imidazol-2-yl)pyridin-3-yl)piperidin-4- yl)methyl)pyrimidin-4-yl)urea |
| 569 | 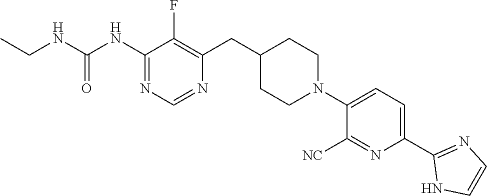 [SMILES: CCNC(=O)Nc1ncnc(CC2CCN(c3ccc(-c4ncc[nH]4)nc3C#N)CC2)c1F] | 1-(6-((1-(2-cyano-6-(1H-imidazol-2- yl)pyridin-3-yl)piperidin-4-yl)methyl)-5- fluoropyrimidin-4-yl)-3-ethylurca |
| 570 | 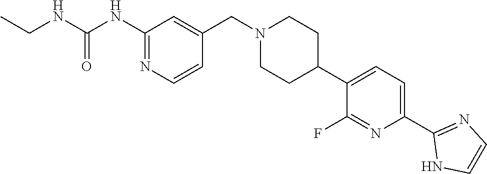 [SMILES: CCNC(=O)Nc1cc(CN2CCC(c3ccc(-c4ncc[nH]4)nc3F)CC2)ccn1] | 1-ethyl-3-(4-((4-(2-fluoro-6-(1H- imidazol-2-yl)pyridin-3-yl)piperidin-1- yl)methyl)pyridin-2-yl)urea |
| 571 | 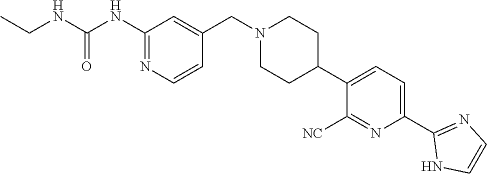 [SMILES: CCNC(=O)Nc1cc(CN2CCC(c3ccc(-c4ncc[nH]4)nc3C#N)CC2)ccn1] | 1-(4-((4-(2-cyano-6-(1H-imidazol-2- yl)pyridin-3-yl)piperidin-1- yl)methyl)pyridin-2-yl)-3-ethylurea |
| 572 | 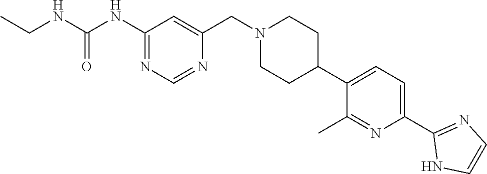 [SMILES: CCNC(=O)Nc1cc(CN2CCC(c3ccc(-c4ncc[nH]4)nc3C)CC2)ncn1] | 1-(6-((4-(6-(1H-imidazol-2-yl)-2- methylpyridin-3-yl)piperidin-1- yl)methyl)pyrimidin-4-yl)-3-ethylurea |
| 573 | 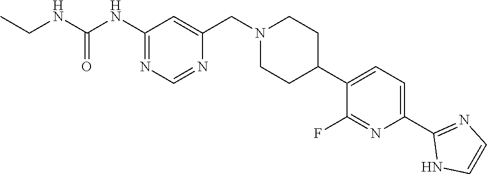 [SMILES: CCNC(=O)Nc1cc(CN2CCC(c3ccc(-c4ncc[nH]4)nc3F)CC2)ncn1] | 1-ethyl-3-(6-((4-(2-fluoro-6-(1H- imidazol-2-yl)pyridin-3-yl)piperidin-1- yl)methyl)pyrimidin-4-yl)urea |
| 574 | 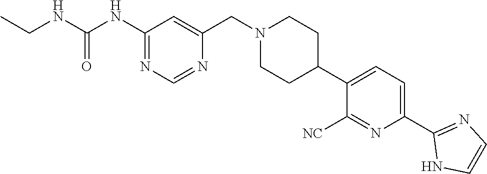 [SMILES: CCNC(=O)Nc1cc(CN2CCC(c3ccc(-c4ncc[nH]4)nc3C#N)CC2)ncn1] | 1-(6-((4-(2-cyano-6-(1H-imidazol-2- yl)pyridin-3-yl)piperidin-1- yl)methyl)pyrimidin-4-yl)-3-ethylurea |
| 575 | 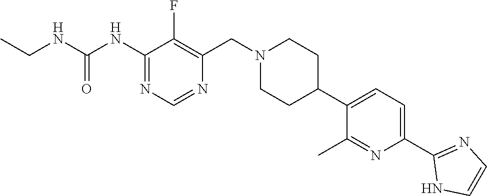 [SMILES: CCNC(=O)Nc1ncnc(CN2CCC(c3ccc(-c4ncc[nH]4)nc3C)CC2)c1F] | 1-(6-((4-(6-(1H-imidazol-2-yl)-2- methylpyridin-3-yl)piperidin-1- yl)methyl)-5-fluoropyrimidin-4-yl)-3- ethylurea |
| 576 | 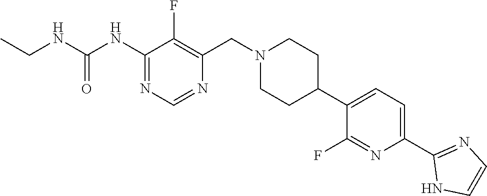 [SMILES: CCNC(=O)Nc1ncnc(CN2CCC(c3ccc(-c4ncc[nH]4)nc3F)CC2)c1F] | 1-ethyl-3-(5-fluoro-6-((4-(2-fluoro-6-(1H- imidazol-2-yl)pyridin-3-yl)piperidin-1- yl)methyl)pyrimidin-4-yl)urea |
| 577 | 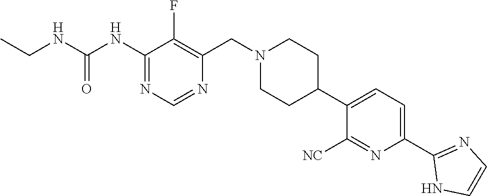 [SMILES: CCNC(=O)Nc1ncnc(CN2CCC(c3ccc(-c4ncc[nH]4)nc3C#N)CC2)c1F] | 1-(6-((4-(2-cyano-6-(1H-imidazol-2- yl)pyridin-3-yl)piperidin-1-yl)methyl)-5- fluoropyrimidin-4-yl)-3-ethylurea |
| 578 | 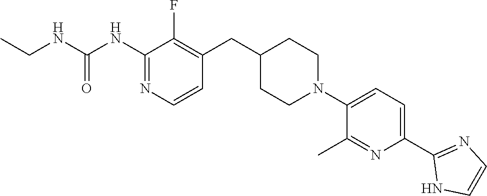 [SMILES: CCNC(=O)Nc1nccc(CC2CCN(c3ccc(-c4ncc[nH]4)nc3C)CC2)c1F] | 1-(4-((1-(6-(1H-imidazol-2-yl)-2- methylpyridin-3-yl)piperidin-4- yl)methyl)-3-fluoropyridin-2-yl)-3- ethylurea |
| 579 | 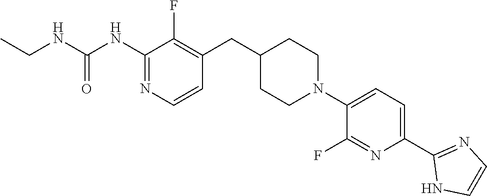 [SMILES: CCNC(=O)Nc1nccc(CC2CCN(c3ccc(-c4ncc[nH]4)nc3F)CC2)c1F] | 1-ethyl-3-(3-fluoro-4-((1-(2-fluoro-6-(1H- imidazol-2-yl)pyridin-3-yl)piperidin-4- yl)methyl)pyridin-2-yl)urea |
| 580 | 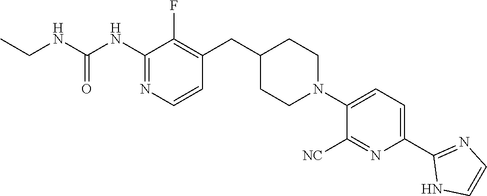 [SMILES: CCNC(=O)Nc1nccc(CC2CCN(c3ccc(-c4ncc[nH]4)nc3C#N)CC2)c1F] | 1-(4-((1-(2-cyano-6-(1H-imidazol-2- yl)pyridin-3-yl)piperidin-4-yl)methyl)-3- fluoropyridin-2-yl)-3-ethylurea |
| 581 | 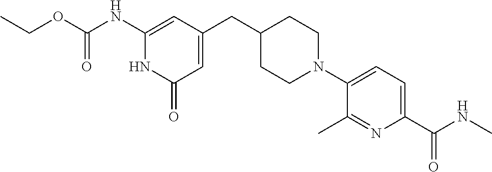 [SMILES: CCOC(=O)Nc1cc(CC2CCN(c3ccc(C(=O)NC)nc3C)CC2)cc(=O)[nH]1] | ethyl (4-((1-(2-methyl-6- (methylcarbamoyl)pyridin-3-yl)piperidin- 4-yl)methyl)-6-oxo-1,6-dihydropyridin-2- yl)carbamate |
| 582 | 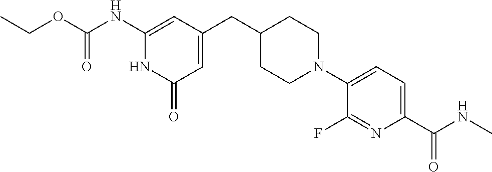 [SMILES: CCOC(=O)Nc1cc(CC2CCN(c3ccc(C(=O)NC)nc3F)CC2)cc(=O)[nH]1] | ethyl (4-((1-(2-fluoro-6- (methylcarbamoyl)pyridin-3-yl)piperidin- 4-yl)methyl)-6-oxo-1,6-dihydropyridin-2- yl)carbamate |
| 583 | 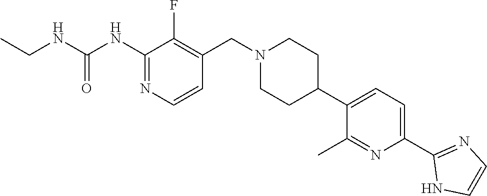 [SMILES: CCNC(=O)Nc1nccc(CN2CCC(c3ccc(-c4ncc[nH]4)nc3C)CC2)c1F] | 1-(4-((4-(6-(1H-imidazol-2-yl)-2- methylpyridin-3-yl)piperidin-1- yl)methyl)-3-fluoropyridin-2-yl)-3- ethylurea |
| 584 | 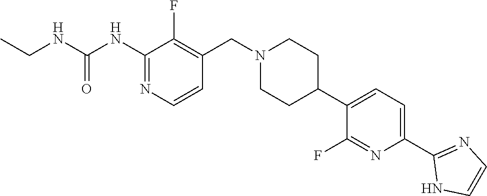 [SMILES: CCNC(=O)Nc1nccc(CN2CCC(c3ccc(-c4ncc[nH]4)nc3F)CC2)c1F] | 1-ethyl-3-(3-fluoro-4-((4-(2-fluoro-6-(1H- imidazol-2-yl)pyridin-3-yl)piperidin-1- yl)methyl)pyridin-2-yl)urea |
| 585 | 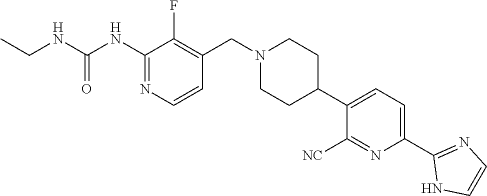 [SMILES: CCNC(=O)Nc1nccc(CN2CCC(c3ccc(-c4ncc[nH]4)nc3C#N)CC2)c1F] | 1-(4-((4-(2-cyano-6-(1H-imidazol-2- yl)pyridin-3-yl)piperidin-1-yl)methyl)-3- fluoropyridin-2-yl)-3-ethylurea |
| 586 | 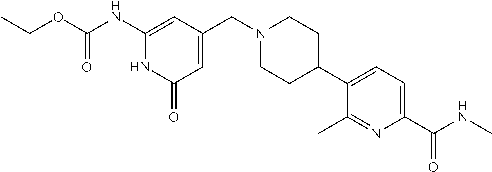 [SMILES: CCOC(=O)Nc1cc(CN2CCC(c3ccc(C(=O)NC)nc3C)CC2)cc(=O)[nH]1] | ethyl (4-((4-(2-methyl-6- (methylcarbamoyl)pyridin-3-yl)piperidin- 1-yl)methyl)-6-oxo-1,6-dihydropyridin-2- yl)carbamate |
| 587 | 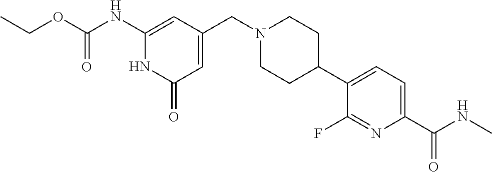 [SMILES: CCOC(=O)Nc1cc(CN2CCC(c3ccc(C(=O)NC)nc3F)CC2)cc(=O)[nH]1] | ethyl (4-((4-(2-fluoro-6- (methylcarbamoyl)pyridin-3-yl)piperidin- 1-yl)methyl)-6-oxo-1,6-dihydropyridin-2- yl)carbamate |
| 588 | 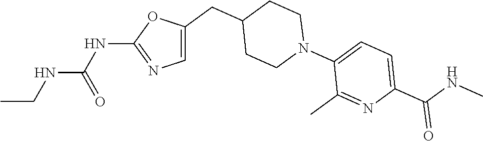 [SMILES: CCNC(=O)Nc1ncc(CC2CCN(c3ccc(C(=O)NC)nc3C)CC2)o1] | 5-(4-((2-(3-ethylureido)oxazol-5- yl)methyl)piperidin-1-yl)-N,6- dimethylpicolinamide |
| 589 | 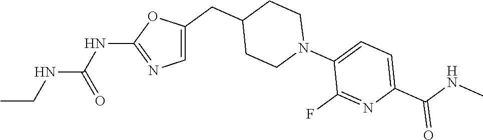 [SMILES: CCNC(=O)Nc1ncc(CC2CCN(c3ccc(C(=O)NC)nc3F)CC2)o1] | 5-(4-((2-(3-ethylureido)oxazol-5- yl)methyl)piperidin-1-yl)-6-fluoro-N- methylpicolinamide |
| 590 | 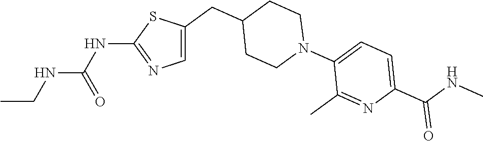 [SMILES: CCNC(=O)Nc1ncc(CC2CCN(c3ccc(C(=O)NC)nc3C)CC2)s1] | 5-(4-((2-(3-ethylureido)thiazol-5- yl)methyl)piperidin-1-yl)-N,6- dimethylpicolinamide |
| 591 | 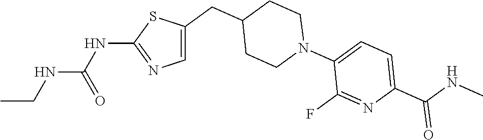 [SMILES: CCNC(=O)Nc1ncc(CC2CCN(c3ccc(C(=O)NC)nc3F)CC2)s1] | 5-(4-((2-(3-ethylureido)thiazol-5- yl)methyl)piperidin-1-yl)-6-fluoro-N- methylpicolinamide |
| 592 | 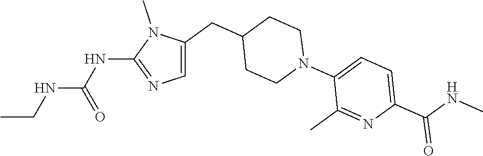 [SMILES: CCNC(=O)Nc1ncc(CC2CCN(c3ccc(C(=O)NC)nc3C)CC2)n1C] | 5-(4-((2-(3-ethylureido)-1-methyl-1H- imidazol-5-yl)methyl)piperidin-1-yl)-N,6- dimethylpicolinamide |
| 593 | 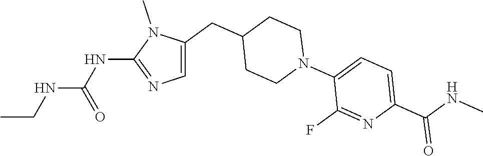 [SMILES: CCNC(=O)Nc1ncc(CC2CCN(c3ccc(C(=O)NC)nc3F)CC2)n1C] | 5-(4-((2-(3-ethylureido)-1-methyl-1H- imidazol-5-yl)methyl)piperidin-1-yl)-6- fluoro-N-methylpicolinamide |
| 594 | 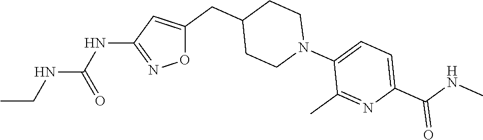 [SMILES: CCNC(=O)Nc1cc(CC2CCN(c3ccc(C(=O)NC)nc3C)CC2)on1] | 5-(4-((3-(3-ethylureido)isoxazol-5- yl)methyl)piperidin-1-yl)-N,6- dimethylpicolinamide |
| 595 | 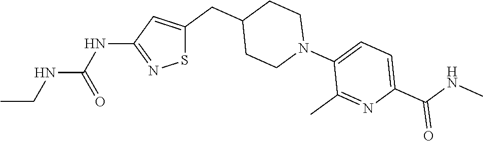 [SMILES: CCNC(=O)Nc1cc(CC2CCN(c3ccc(C(=O)NC)nc3C)CC2)sn1] | 5-(4-((3-(3-ethylureido)isothiazol-5- yl)methyl)piperidin-1-yl)-N,6- dimethylpicolinamide |
| 596 | 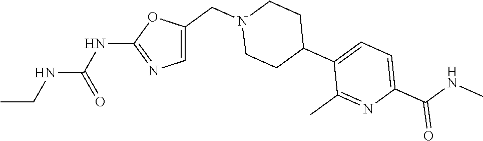 [SMILES: CCNC(=O)Nc1ncc(CN2CCC(c3ccc(C(=O)NC)nc3C)CC2)o1] | 5-(1-((2-(3-ethylureido)oxazol-5- yl)methyl)piperidin-4-yl)-N,6- dimethylpicolinamide |
| 597 | 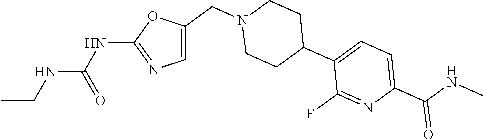 [SMILES: CCNC(=O)Nc1ncc(CN2CCC(c3ccc(C(=O)NC)nc3F)CC2)o1] | 5-(1-((2-(3-ethylureido)oxazol-5- yl)methyl)piperidin-4-yl)-6-fluoro-N- methylpicolinamide |
| 598 | 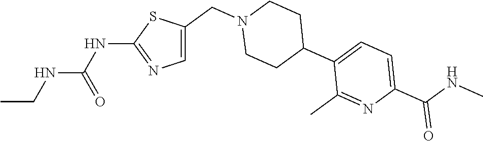 [SMILES: CCNC(=O)Nc1ncc(CN2CCC(c3ccc(C(=O)NC)nc3C)CC2)s1] | 5-(1-((2-(3-ethylureido)thiazol-5- yl)methyl)piperidin-4-yl)-N,6- dimethylpicolinamide |
| 599 | 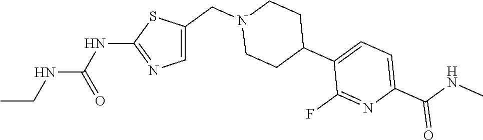 [SMILES: CCNC(=O)Nc1ncc(CN2CCC(c3ccc(C(=O)NC)nc3F)CC2)s1] | 5-(1-((2-(3-ethylureido)thiazol-5- yl)methyl)piperidin-4-yl)-6-fluoro-N- methylpicolinamide |
| 600 | 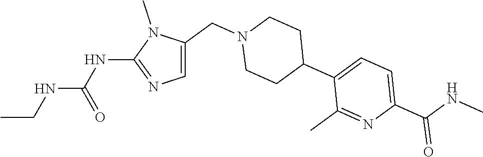 [SMILES: CCNC(=O)Nc1ncc(CN2CCC(c3ccc(C(=O)NC)nc3C)CC2)n1C] | 5-(1-((2-(3-ethylureido)-1-methyl-1H- imidazol-5-yl)methyl)piperidin-4-yl)-N,6- dimethylpicolinamide |
| 601 | 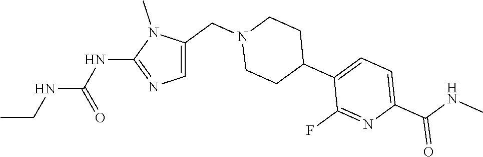 [SMILES: CCNC(=O)Nc1ncc(CN2CCC(c3ccc(C(=O)NC)nc3F)CC2)n1C] | 5-(1-((2-(3-ethylureido)-1-methyl-1H- imidazol-5-yl)methyl)piperidin-4-yl)-6- fluoro-N-methylpicolinamide |
| 602 | 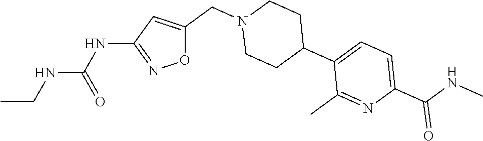 [SMILES: CCNC(=O)Nc1cc(CN2CCC(c3ccc(C(=O)NC)nc3C)CC2)on1] | 5-(1-((3-(3-ethylureido)isoxazol-5- yl)methyl)piperidin-4-yl)-N,6- dimethylpicolinamide |
| 603 | 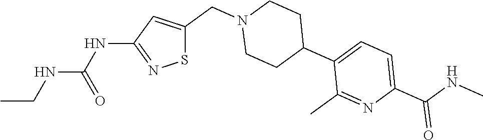 [SMILES: CCNC(=O)Nc1cc(CN2CCC(c3ccc(C(=O)NC)nc3C)CC2)sn1] | 5-(1-((3-(3-ethylureido)isothiazol-5- yl)methyl)piperidin-4-yl)-N,6- dimethylpicolinamide |
| 604 | 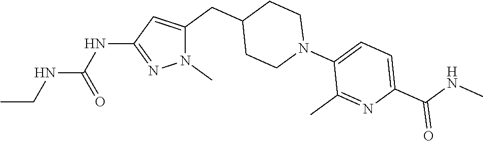 [SMILES: CCNC(=O)Nc1cc(CC2CCN(c3ccc(C(=O)NC)nc3C)CC2)n(C)n1] | 5-(4-((3-(3-ethylureido)-1-methyl-1H- pyrazol-5-yl)methyl)piperidin-1-yl)-N,6- dimethylpicolinamide |
| 605 | 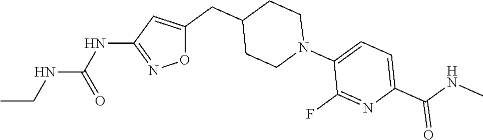 [SMILES: CCNC(=O)Nc1cc(CC2CCN(c3ccc(C(=O)NC)nc3F)CC2)on1] | 5-(4-((3-(3-ethylureido)isoxazol-5- yl)methyl)piperidin-1-yl)-6-fluoro-N- methylpicolinamide |
| 606 | 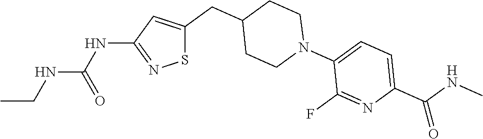 | 5-(4-((3-(3-ethylureido)isothiazol-5- yl)methyl)piperidin-1-yl)-6-fluoro-N- methylpicolinamide |
| 607 | 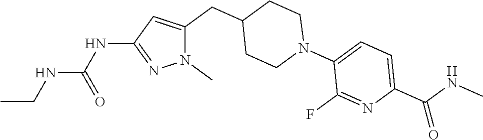 | 5-(4-((3-(3-ethylureido)-1-methyl-1H- pyrazol-5-yl)methyl)piperidin-1-yl)-6- fluoro-N-methylpicolinamide |
| 608 | 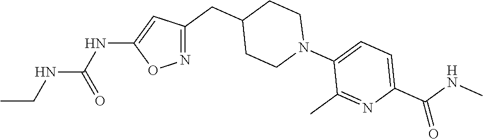 | 5-(4-((5-(3-ethylureido)isoxazol-3- yl)methyl)piperidin-1-yl)-N,6- dimethylpicolinamide |
| 609 | 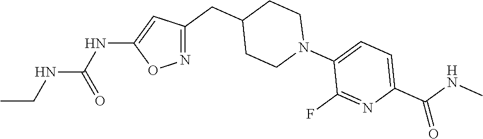 | 5-(4-((5-(3-ethylureido)isoxazol-3- yl)methyl)piperidin-1-yl)-6-fluoro-N- methylpicolinamide |
| 610 | 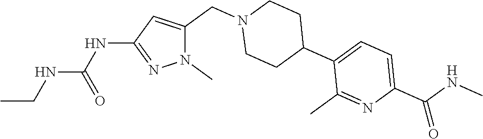 | S-(1-((3-(3-ethylureido)-1-methyl-1H- pyrazol-5-yl)methyl)piperidin-4-yl)-N,6- dimethylpicolinamide |
| 611 | 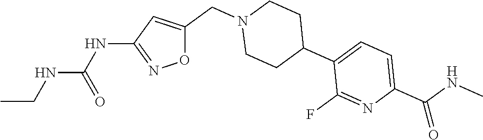 | 5-(1-((3-(3-ethylureido)isoxazol-5- yl)methyl)piperidin-4-yl)-6-fluoro-N- methylpicolinamide |
| 612 | 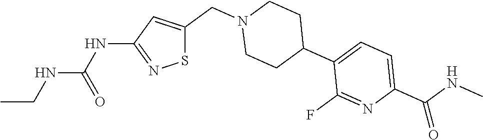 | 5-(1-((3-(3-ethylureido)isothiazol-5- yl)methyl)piperidin-4-yl)-6-fluoro-N- methylpicolinamide |
| 613 | 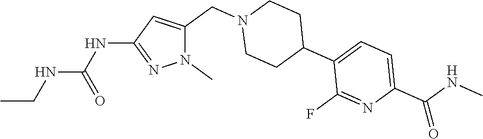 | 5-(1-((3-(3-ethylureido)-1-methyl-1H- pyrazol-5-yl)methyl)piperidin-4-yl)-6- fluoro-N-methylpicolinamide |
| 614 | 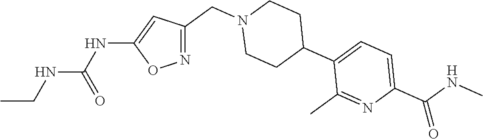 | 5-(1-((5-(3-ethylureido)isoxazol-3- yl)methyl)piperidin-4-yl)-N,6- dimethylpicolinamide |
| 615 | 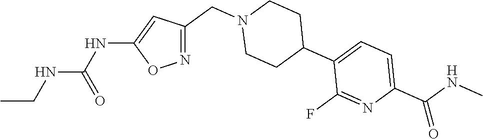 | 5-(1-((5-(3-ethylureido)isoxazol-3- yl)methyl)piperidin-4-yl)-6-fluoro-N- methylpicolinamide |
| 616 | 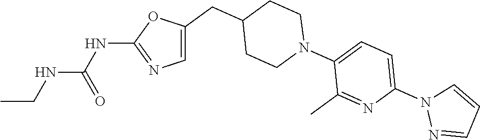 [SMILES: CCNC(=O)Nc1ncc(CC2CCN(c3ccc(-n4cccn4)nc3C)CC2)o1] | 1-ethyl-3-(5-((1-(2-methyl-6-(1H- pyrazol-1-yl)pyridin-3-yl)piperidin-4- yl)methyl)oxazol-2-yl)urea |
| 617 | 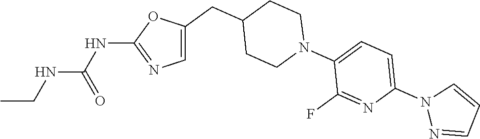 [SMILES: CCNC(=O)Nc1ncc(CC2CCN(c3ccc(-n4cccn4)nc3F)CC2)o1] | 1-ethyl-3-(5-((1-(2-fluoro-6-(1H-pyrazol- 1-yl)pyridin-3-yl)piperidin-4- yl)methyl)oxazol-2-yl)urea |
| 618 | 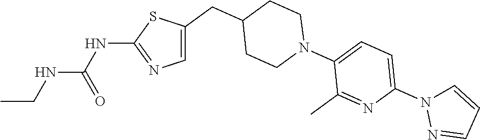 [SMILES: CCNC(=O)Nc1ncc(CC2CCN(c3ccc(-n4cccn4)nc3C)CC2)s1] | 1-ethyl-3-(5-((1-(2-methyl-6-(1H- pyrazol-1-yl)pyridin-3-yl)piperidin-4- yl)methyl)thiazol-2-yl)urea |
| 619 | 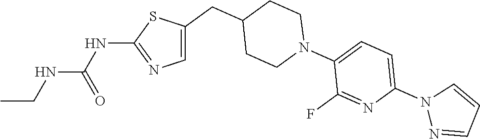 [SMILES: CCNC(=O)Nc1ncc(CC2CCN(c3ccc(-n4cccn4)nc3F)CC2)s1] | 1-ethyl-3-(5-((1-(2-fluoro-6-(1H-pyrazol- 1-yl)pyridin-3-yl)piperidin-4- yl)methyl)thiazol-2-yl)urea |
| 620 | 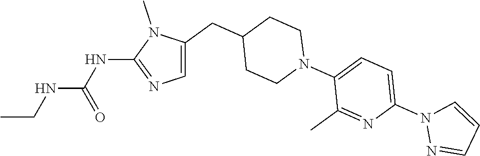 [SMILES: CCNC(=O)Nc1ncc(CC2CCN(c3ccc(-n4cccn4)nc3C)CC2)n1C] | 1-ethyl-3-(1-methyl-5-((1-(2-methyl-6- (1H-pyrazol-1-yl)pyridin-3-yl)piperidin- 4-yl)methyl)-1H-imidazol-2-yl)urea |
| 621 | 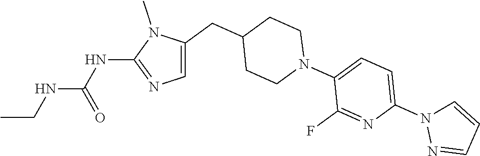 [SMILES: CCNC(=O)Nc1ncc(CC2CCN(c3ccc(-n4cccn4)nc3F)CC2)n1C] | 1-ethyl-3-(5-((1-(2-fluoro-6-(1H-pyrazol- 1-yl)pyridin-3-yl)piperidin-4-yl)methyl)- 1-methyl-1H-imidazol-2-yl)urea |
| 622 | 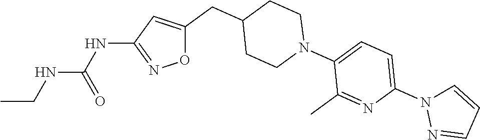 [SMILES: CCNC(=O)Nc1cc(CC2CCN(c3ccc(-n4cccn4)nc3C)CC2)on1] | 1-ethyl-3-(5-((1-(2-methyl-6-(1H- pyrazol-1-yl)pyridin-3-yl)piperidin-4- yl)methyl)isoxazol-3-yl)urea |
| 623 | 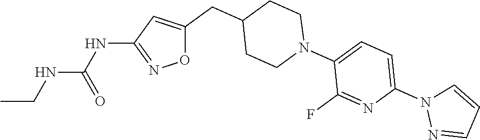 [SMILES: CCNC(=O)Nc1cc(CC2CCN(c3ccc(-n4cccn4)nc3F)CC2)on1] | 1-ethyl-3-(5-((1-(2-fluoro-6-(1H-pyrazol- 1-yl)pyridin-3-yl)piperidin-4- yl)methyl)isoxazol-3-yl)urea |
| 624 | 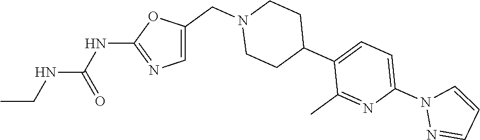 [SMILES: CCNC(=O)Nc1ncc(CN2CCC(c3ccc(-n4cccn4)nc3C)CC2)o1] | 1-ethyl-3-(5-((4-(2-methyl-6-(1H- pyrazol-1-yl)pyridin-3-yl)piperidin-1- yl)methyl)oxazol-2-yl)urea |
| 625 | 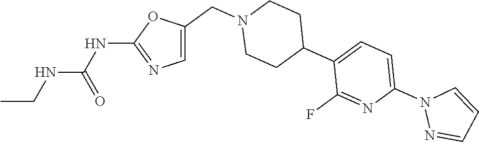 [SMILES: CCNC(=O)Nc1ncc(CN2CCC(c3ccc(-n4cccn4)nc3F)CC2)o1] | 1-ethyl-3-(5-((4-(2-fluoro-6-(1H-pyrazol- 1-yl)pyridin-3-yl)piperidin-1- yl)methyl)oxazol-2-yl)urea |
| 626 | 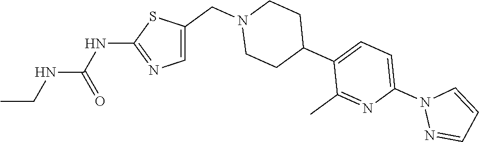 [SMILES: CCNC(=O)Nc1ncc(CN2CCC(c3ccc(-n4cccn4)nc3C)CC2)s1] | 1-ethyl-3-(5-((4-(2-methyl-6-(1H- pyrazol-1-yl)pyridin-3-yl)piperidin-1- yl)methyl)thiazol-2-yl)urea |
| 627 | 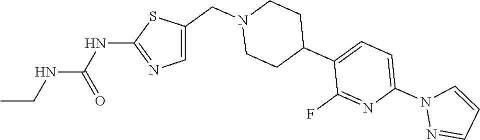 [SMILES: CCNC(=O)Nc1ncc(CN2CCC(c3ccc(-n4cccn4)nc3F)CC2)s1] | 1-ethyl-3-(5-((4-(2-fluoro-6-(1H-pyrazol- 1-yl)pyridin-3-yl)piperidin-1- yl)methyl)thiazol-2-yl)urea |
| 628 | 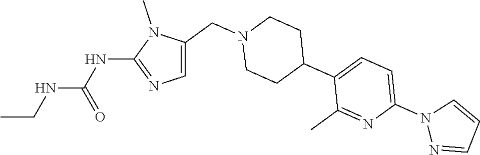 [SMILES: CCNC(=O)Nc1ncc(CN2CCC(c3ccc(-n4cccn4)nc3C)CC2)n1C] | 1-ethyl-3-(1-methyl-5-((4-(2-methyl-6- (1H-pyrazol-1-yl)pyridin-3-yl)piperidin- 1-yl)methyl)-1H-imidazol-2-yl)urea |
| 629 | 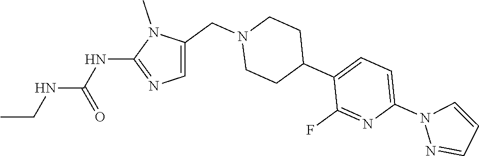 [SMILES: CCNC(=O)Nc1ncc(CN2CCC(c3ccc(-n4cccn4)nc3F)CC2)n1C] | 1-ethyl-3-(5-((4-(2-fluoro-6-(1H-pyrazol- 1-yl)pyridin-3-yl)piperidin-1-yl)methyl)- 1-methyl-1H-imidazol-2-yl)urea |
| 630 | 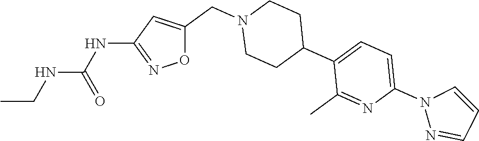 [SMILES: CCNC(=O)Nc1cc(CN2CCC(c3ccc(-n4cccn4)nc3C)CC2)on1] | 1-ethyl-3-(5-((4-(2-methyl-6-(1H- pyrazol-1-yl)pyridin-3-yl)piperidin-1- yl)methyl)isoxazol-3-yl)urea |
| 631 | 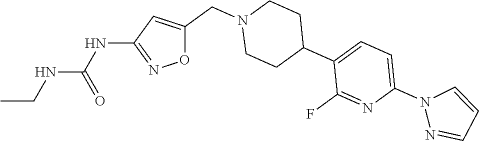 [SMILES: CCNC(=O)Nc1cc(CN2CCC(c3ccc(-n4cccn4)nc3F)CC2)on1] | 1-ethyl-3-(5-((4-(2-fluoro-6-(1H-pyrazol- 1-yl)pyridin-3-yl)piperidin-1- yl)methyl)isoxazol-3-yl)urea |
| 632 | 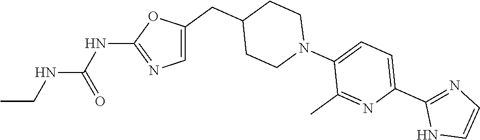 [SMILES: CCNC(=O)Nc1ncc(CC2CCN(c3ccc(-c4ncc[nH]4)nc3C)CC2)o1] | 1-(5-((1-(6-(1H-imidazol-2-yl)-2- methylpyridin-3-yl)piperidin-4- yl)methyl)oxazol-2-yl)-3-ethylurea |
| 633 | 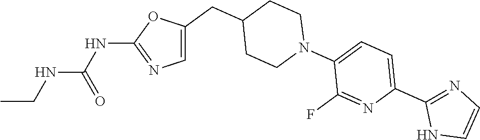 [SMILES: CCNC(=O)Nc1ncc(CC2CCN(c3ccc(-c4ncc[nH]4)nc3F)CC2)o1] | 1-ethyl-3-(5-((1-(2-fluoro-6-(1H- imidazol-2-yl)pyridin-3-yl)piperidin-4- yl)methyl)oxazol-2-yl)urea |
| 634 | 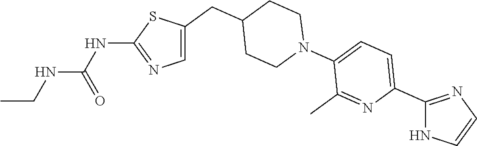 [SMILES: CCNC(=O)Nc1ncc(CC2CCN(c3ccc(-c4ncc[nH]4)nc3C)CC2)s1] | 1-(5-((1-(6-(1H-imidazol-2-yl)-2- methylpyridin-3-yl)piperidin-4- yl)methyl)thiazol-2-yl)-3-ethylurea |
| 635 | 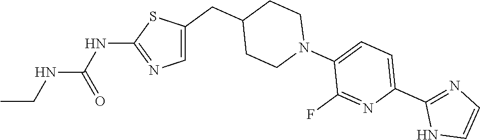 [SMILES: CCNC(=O)Nc1ncc(CC2CCN(c3ccc(-c4ncc[nH]4)nc3F)CC2)s1] | 1-ethyl-3-(5-((1-(2-fluoro-6-(1H- imidazol-2-yl)pyridin-3-yl)piperidin-4- yl)methyl)thiazol-2-yl)urea |
| 636 | 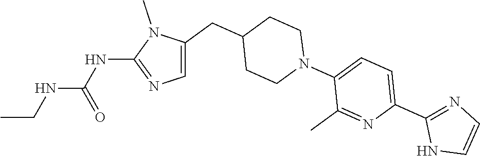 [SMILES: CCNC(=O)Nc1ncc(CC2CCN(c3ccc(-c4ncc[nH]4)nc3C)CC2)n1C] | 1-(5-((1-(6-(1H-imidazol-2-yl)-2- methylpyridin-3-yl)piperidin-4- yl)methyl)-1-methyl-1H-imidazol-2-yl)- 3-ethylurea |
| 637 | 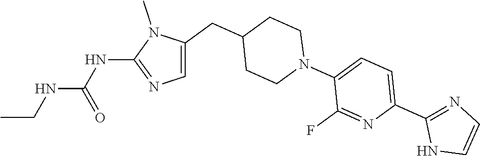 [SMILES: CCNC(=O)Nc1ncc(CC2CCN(c3ccc(-c4ncc[nH]4)nc3F)CC2)n1C] | 1-ethyl-3-(5-((1-(2-fluoro-6-(1H- imidazol-2-yl)pyridin-3-yl)piperidin-4- yl)methyl)-1-methyl-1H-imidazol-2- yl)urea |
| 638 | 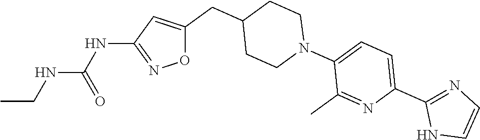 [SMILES: CCNC(=O)Nc1cc(CC2CCN(c3ccc(-c4ncc[nH]4)nc3C)CC2)on1] | 1-(5-((1-(6-(1H-imidazol-2-yl)-2- methylpyridin-3-yl)piperidin-4- yl)methyl)isoxazol-3-yl)-3-ethylurea |
| 639 | 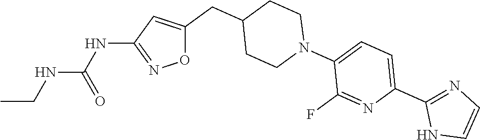 [SMILES: CCNC(=O)Nc1cc(CC2CCN(c3ccc(-c4ncc[nH]4)nc3F)CC2)on1] | 1-ethyl-3-(5-((1-(2-fluoro-6-(1H- imidazol-2-yl)pyridin-3-yl)piperidin-4- yl)methyl)isoxazol-3-yl)urea |
| 640 | 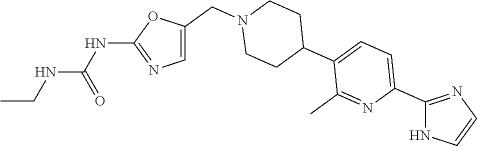 [SMILES: CCNC(=O)Nc1ncc(CN2CCC(c3ccc(-c4ncc[nH]4)nc3C)CC2)o1] | 1-(5-((4-(6-(1H-imidazol-2-yl)-2- methylpyridin-3-yl)piperidin-1- yl)methyl)oxazol-2-yl)-3-ethylurea |
| 641 | 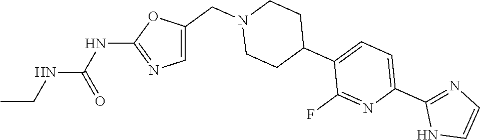 [SMILES: CCNC(=O)Nc1ncc(CN2CCC(c3ccc(-c4ncc[nH]4)nc3F)CC2)o1] | 1-ethyl-3-(5-((4-(2-fluoro-6-(1H- imidazol-2-yl)pyridin-3-yl)piperidin-1- yl)methyl)oxazol-2-yl)urea |
| 642 | 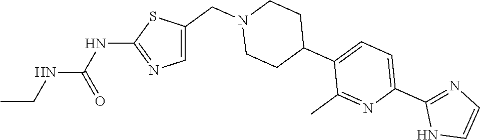 [SMILES: CCNC(=O)Nc1ncc(CN2CCC(c3ccc(-c4ncc[nH]4)nc3C)CC2)s1] | 1-(5-((4-(6-(1H-imidazol-2-yl)-2- methylpyridin-3-yl)piperidin-1- yl)methyl)thiazol-2-yl)-3-ethylurea |
| 643 | 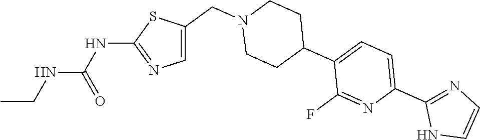 [SMILES: CCNC(=O)Nc1ncc(CN2CCC(c3ccc(-c4ncc[nH]4)nc3F)CC2)s1] | 1-ethyl-3-(5-((4-(2-fluoro-6-(1H- imidazol-2-yl)pyridin-3-yl)piperidin-1- yl)methyl)thiazol-2-yl)urea |
| 644 | 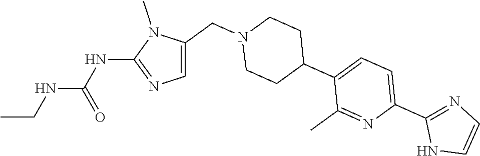 [SMILES: CCNC(=O)Nc1ncc(CN2CCC(c3ccc(-c4ncc[nH]4)nc3C)CC2)n1C] | 1-(5-((4-(6-(1H-imidazol-2-yl)-2- methylpyridin-3-yl)piperidin-1- yl)methyl)-1-methyl-1H-imidazol-2-yl)- 3-ethylurea |
| 645 | 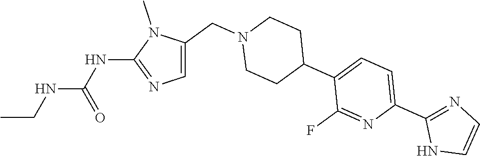 [SMILES: CCNC(=O)Nc1ncc(CN2CCC(c3ccc(-c4ncc[nH]4)nc3F)CC2)n1C] | 1-ethyl-3-(5-((4-(2-fluoro-6-(1H- imidazol-2-yl)pyridin-3-yl)piperidin-1- yl)methyl)-1-methyl-1H-imidazol-2- yl)urea |
| 646 | 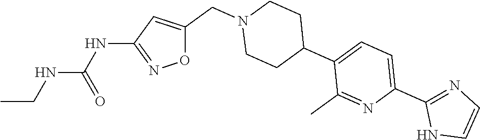 [SMILES: CCNC(=O)Nc1cc(CN2CCC(c3ccc(-c4ncc[nH]4)nc3C)CC2)on1] | 1-(5-((4-(6-(1H-imidazol-2-yl)-2- methylpyridin-3-yl)piperidin-1- yl)methyl)isoxazol-3-yl)-3-ethylurea |
| 647 | 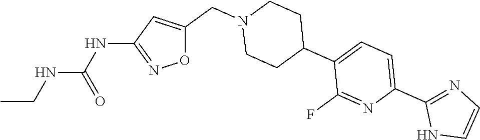 [SMILES: CCNC(=O)Nc1cc(CN2CCC(c3ccc(-c4ncc[nH]4)nc3F)CC2)on1] | 1-ethyl-3-(5-((4-(2-fluoro-6-(1H- imidazol-2-yl)pyridin-3-yl)piperidin-1- yl)methyl)isoxazol-3-yl)urea |
| 648 | 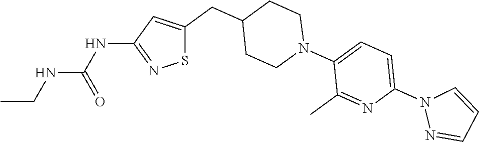 [SMILES: CCNC(=O)Nc1cc(CC2CCN(c3ccc(-n4cccn4)nc3C)CC2)sn1] | 1-ethyl-3-(5-((1-(2-methyl-6-(1H- pyrazol-1-yl)pyridin-3-yl)piperidin-4- yl)methyl)isothiazol-3-yl)urea |
| 649 | 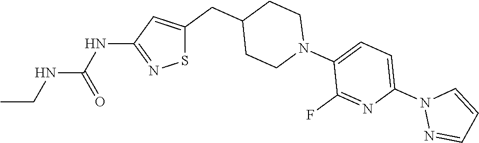 [SMILES: CCNC(=O)Nc1cc(CC2CCN(c3ccc(-n4cccn4)nc3F)CC2)sn1] | 1-ethyl-3-(5-((1-(2-fluoro-6-(1H-pyrazol- 1-yl)pyridin-3-yl)piperidin-4- yl)methyl)isothiazol-3-yl)urea |
| 650 | 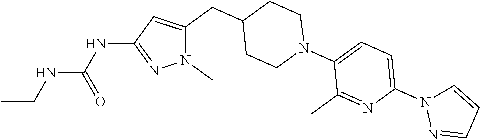 [SMILES: CCNC(=O)Nc1cc(CC2CCN(c3ccc(-n4cccn4)nc3C)CC2)n(C)n1] | 1-ethyl-3-(1-methyl-5-((1-(2-methyl-6- (1H-pyrazol-1-yl)pyridin-3-yl)piperidin- 4-yl)methyl)-1H-pyrazol-3-yl)urea |
| 651 | 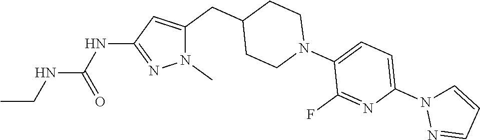 [SMILES: CCNC(=O)Nc1cc(CC2CCN(c3ccc(-n4cccn4)nc3F)CC2)n(C)n1] | 1-ethyl-3-(5-((1-(2-fluoro-6-(1H-pyrazol- 1-yl)pyridin-3-yl)piperidin-4-yl)methyl)- 1-methyl-1H-pyrazol-3-yl)urea |
| 652 | 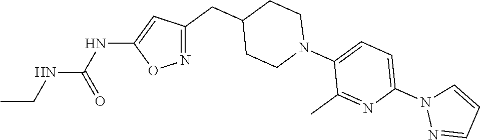 [SMILES: CCNC(=O)Nc1cc(CC2CCN(c3ccc(-n4cccn4)nc3C)CC2)no1] | 1-ethyl-3-(3-((1-(2-methyl-6-(1H- pyrazol-1-yl)pyridin-3-yl)piperidin-4- yl)methyl)isoxazol-5-yl)urea |
| 653 | 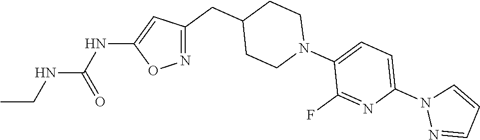 [SMILES: CCNC(=O)Nc1cc(CC2CCN(c3ccc(-n4cccn4)nc3F)CC2)no1] | 1-ethyl-3-(3-((1-(2-fluoro-6-(1H-pyrazol- 1-yl)pyridin-3-yl)piperidin-4- yl)methyl)isoxazol-5-yl)urea |
| 654 | 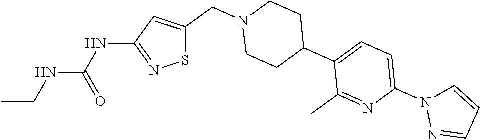 [SMILES: CCNC(=O)Nc1cc(CN2CCC(c3ccc(-n4cccn4)nc3C)CC2)sn1] | 1-ethyl-3-(5-((4-(2-methyl-6-(1H- pyrazol-1-yl)pyridin-3-yl)piperidin-1- yl)methyl)isothiazol-3-yl)urea |
| 655 | 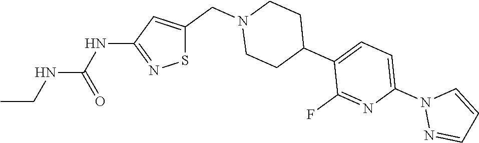 [SMILES: CCNC(=O)Nc1cc(CN2CCC(c3ccc(-n4cccn4)nc3F)CC2)sn1] | 1-ethyl-3-(5-((4-(2-fluoro-6-(1H-pyrazol- 1-yl)pyridin-3-yl)piperidin-1- yl)methyl)isothiazol-3-yl)urea |
| 656 | 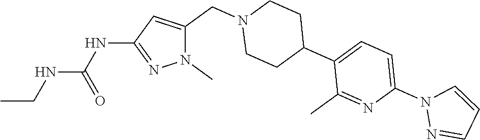 [SMILES: CCNC(=O)Nc1cc(CN2CCC(c3ccc(-n4cccn4)nc3C)CC2)n(C)n1] | 1-ethyl-3-(1-methyl-5-((4-(2-methyl-6- (1H-pyrazol-1-yl)pyridin-3-yl)piperidin- 1-yl)methyl)-1H-pyrazol-3-yl)urea |
| 657 | 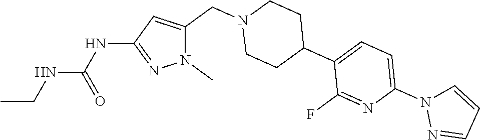 [SMILES: CCNC(=O)Nc1cc(CN2CCC(c3ccc(-n4cccn4)nc3F)CC2)n(C)n1] | 1-ethyl-3-(5-((4-(2-fluoro-6-(1H-pyrazol- 1-yl)pyridin-3-yl)piperidin-1-yl)methyl)- 1-methyl-1H-pyrazol-3-yl)urea |
| 658 | 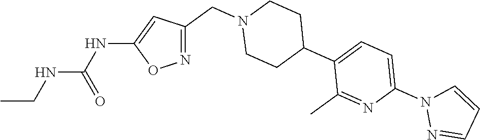 [SMILES: CCNC(=O)Nc1cc(CN2CCC(c3ccc(-n4cccn4)nc3C)CC2)no1] | 1-ethyl-3-(3-((4-(2-methyl-6-(1H- pyrazol-1-yl)pyridin-3-yl)piperidin-1- yl)methyl)isoxazol-5-yl)urea |
| 659 | 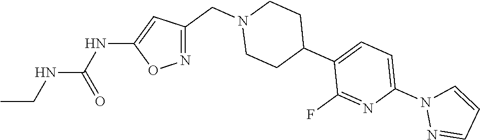 [SMILES: CCNC(=O)Nc1cc(CN2CCC(c3ccc(-n4cccn4)nc3F)CC2)no1] | 1-ethyl-3-(3-((4-(2-fluoro-6-(1H-pyrazol- 1-yl)pyridin-3-yl)piperidin-1- yl)methyl)isoxazol-5-yl)urea |
| 660 | 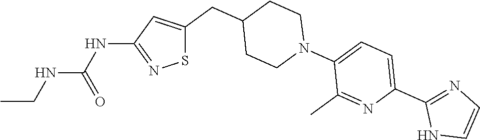 [SMILES: CCNC(=O)Nc1cc(CC2CCN(c3ccc(-c4ncc[nH]4)nc3C)CC2)sn1] | 1-(5-((1-(6-(1H-imidazol-2-yl)-2- methylpyridin-3-yl)piperidin-4- yl)methyl)isothiazol-3-yl)-3-ethylurea |
| 661 | 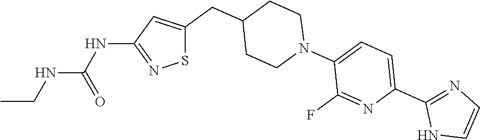 [SMILES: CCNC(=O)Nc1cc(CC2CCN(c3ccc(-c4ncc[nH]4)nc3F)CC2)sn1] | 1-ethyl-3-(5-((1-(2-fluoro-6-(1H- imidazol-2-yl)pyridin-3-yl)piperidin-4- yl)methyl)isothiazol-3-yl)urea |
| 662 | 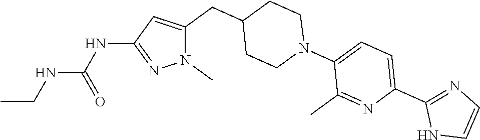 [SMILES: CCNC(=O)Nc1cc(CC2CCN(c3ccc(-c4ncc[nH]4)nc3C)CC2)n(C)n1] | 1-(5-((1-(6-(1H-imidazol-2-yl)-2- methylpyridin-3-yl)piperidin-4- yl)methyl)-1-methyl-1H-pyrazol-3-yl)-3- ethylurea |
| 663 | 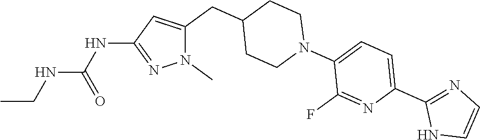 [SMILES: CCNC(=O)Nc1cc(CC2CCN(c3ccc(-c4ncc[nH]4)nc3F)CC2)n(C)n1] | 1-ethyl-3-(5-((1-(2-fluoro-6-(1H- imidazol-2-yl)pyridin-3-yl)piperidin-4- yl)methyl)-1-methyl-1H-pyrazol-3- yl)urea |
| 664 | 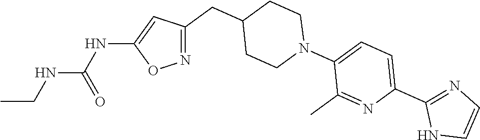 [SMILES: CCNC(=O)Nc1cc(CC2CCN(c3ccc(-c4ncc[nH]4)nc3C)CC2)no1] | 1-(3-((1-(6-(1H-imidazol-2-yl)-2- methylpyridin-3-yl)piperidin-4- yl)methyl)isoxazol-5-yl)-3-ethylurea |
| 665 | 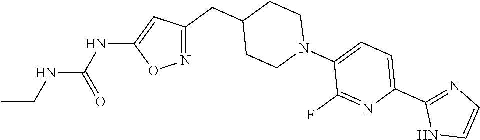 [SMILES: CCNC(=O)Nc1cc(CC2CCN(c3ccc(-c4ncc[nH]4)nc3F)CC2)no1] | 1-ethyl-3-(3-((1-(2-fluoro-6-(1H- imidazol-2-yl)pyridin-3-yl)piperidin-4- yl)methyl)isoxazol-5-yl)urea |
| 666 | 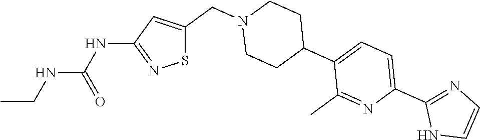 [SMILES: CCNC(=O)Nc1cc(CN2CCC(c3ccc(-c4ncc[nH]4)nc3C)CC2)sn1] | 1-(5-((4-(6-(1H-imidazol-2-yl)-2- methylpyridin-3-yl)piperidin-1- yl)methyl)isothiazol-3-yl)-3-ethylurea |
| 667 | 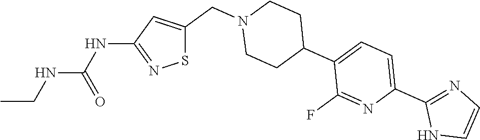 [SMILES: CCNC(=O)Nc1cc(CN2CCC(c3ccc(-c4ncc[nH]4)nc3F)CC2)sn1] | 1-ethyl-3-(5-((4-(2-fluoro-6-(1H- imidazol-2-yl)pyridin-3-yl)piperidin-1- yl)methyl)isothiazol-3-yl)urea |
| 668 | 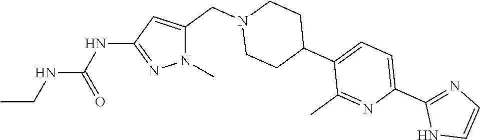 [SMILES: CCNC(=O)Nc1cc(CN2CCC(c3ccc(-c4ncc[nH]4)nc3C)CC2)n(C)n1] | 1-(5-((4-(6-(1H-imidazol-2-yl)-2- methylpyridin-3-yl)piperidin-1- yl)methyl)-1-methyl-1H-pyrazol-3-yl)-3- ethylurea |
| 669 | 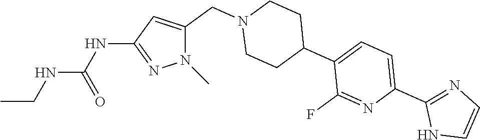 [SMILES: CCNC(=O)Nc1cc(CN2CCC(c3ccc(-c4ncc[nH]4)nc3F)CC2)n(C)n1] | 1-ethyl-3-(5-((4-(2-fluoro-6-(1H- imidazol-2-yl)pyridin-3-yl)piperidin-1- yl)methyl)-1-methyl-1H-pyrazol-3- yl)urea |
| 670 | 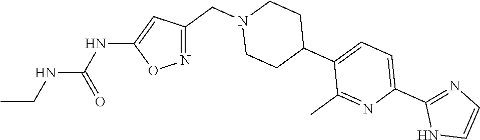 [SMILES: CCNC(=O)Nc1cc(CN2CCC(c3ccc(-c4ncc[nH]4)nc3C)CC2)no1] | 1-(3-((4-(6-(1H-imidazol-2-yl)-2- methylpyridin-3-yl)piperidin-1- yl)methyl)isoxazol-5-yl)-3-ethylurea |
| 671 | 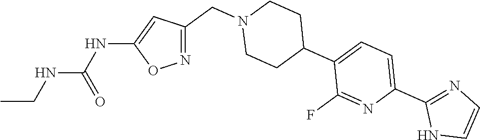 [SMILES: CCNC(=O)Nc1cc(CN2CCC(c3ccc(-c4ncc[nH]4)nc3F)CC2)no1] | 1-ethyl-3-(3-((4-(2-fluoro-6-(1H- imidazol-2-yl)pyridin-3-yl)piperidin-1- yl)methyl)isoxazol-5-yl)urea |
| 672 | 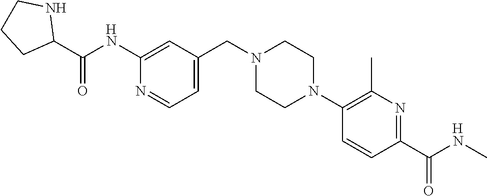 | N,6-dimethyl-5-(4-((2-(pyrrolidine-2- carboxamido)pyridin-4- yl)methyl)piperazin-1-yl)picolinamide |
| 673 | 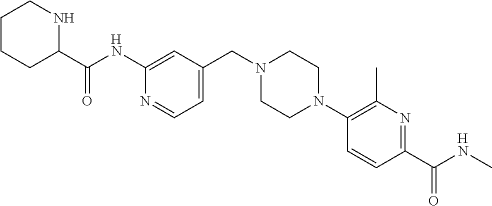 | N,6-dimethyl-5-(4-((2-(piperidine-2- carboxamido)pyridin-4- yl)methyl)piperazin-1-yl)picolinamide |
| 674 | 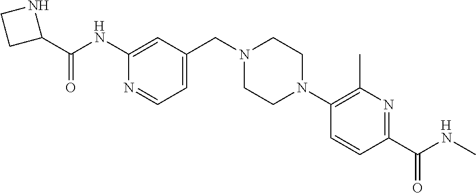 | 5-(4-((2-(azetidine-2- carboxamido)pyridin-4- yl)methyl)piperazin-1-yl)-N,6- dimethylpicolinamide |
| 675 | 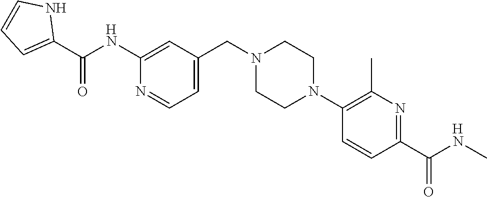 | 5-(4-((2-(1H-pyrrole-2- carboxamido)pyridin-4- yl)methyl)piperazin-1-yl)-N,6- dimethylpicolinamide |
| 676 | 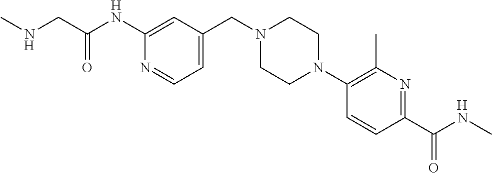 | N,6-dimethyl-5-(4-((2-(2- (methylamino)acetamido)pyridin-4- yl)methyl)piperazin-1-yl)picolinamide |
| 677 | 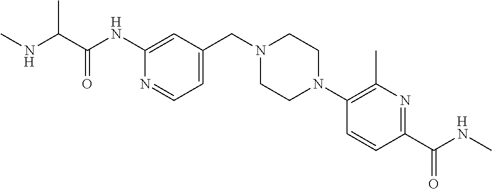 | N,6-dimethyl-5-(4-((2-(2- (methylamino)propanamido)pyridin-4- yl)methyl)piperazin-1-yl)picolinamide |
| 678 | 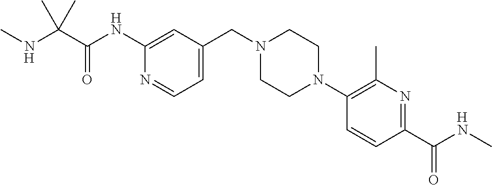 | N,6-dimethyl-5-(4-((2-(2-methyl-2- (methylamino)propanamido)pyridin-4- yl)methyl)piperazin-1-yl)picolinamide |
| 679 | 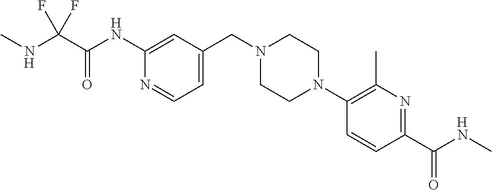 | 5-(4-((2-(2,2-difluoro-2- (methylamino)acetamido)pyridin-4- yl)methyl)piperazin-1-yl)-N,6- dimethylpicolinamide |
| 680 | 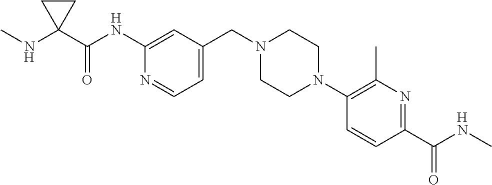 | N,6-dimethyl-5-(4-((2-(1- (methylamino)cyclopropanecarboxamido) pyridin-4-yl)methyl)piperazin-1- yl)picolinamide |

In some embodiments, the compound is a pharmaceutically acceptable salt of any one of the compounds described in Table 1.

The neutral compounds of Formula (I) may be converted to the corresponding pharmaceutically acceptable salts of the compounds using techniques in the art (e.g., by saponification of an ester to the carboxylic acid salt, or by hydrolyzing an amide to form a corresponding carboxylic acid and then converting the carboxylic acid to a carboxylic acid salt).

In some embodiments, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³⁵S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁴S, and/or ³⁵S labeled compound, can be prepared using any of a variety of art-recognised techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. In some embodiments, the molecular weight of the compound will be less than 900, for example less than 800, or less than 750, or less than 700, or less than 650 daltons. For example, the molecular weight is less than 600 and, for example, is 550 daltons or less.

A suitable pharmaceutically acceptable salt of a compound of the disclosure is, for example, an acid addition salt of a compound of the disclosure which is sufficiently basic, for example, an acid addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris(2hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413, Cahn and Ingold, J. Chem. Soc. 1951 (London), 612; Cahn et al., Experientia 1956, 12, 81; Cahn, J. Chem. Fduc. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are nonsuperimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R and S sequencing rules of Cahn and Prelog, or by the manner in which the molecule rotates the plane of polarized light and designated as dextrorotatory or levorotatory (i.e., as (+) or ( )isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E and Z isomers). It is to be understood that the present disclosure encompasses all optical, diastereoisomers and geometric isomers and mixtures thereof that possess (1) PARP and/or (2) PARP-1 inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess (1) PARP and/or (2) PARP-1 inhibitory activity.

It is also to be understood that certain compounds of any one of the Formulae disclosed herein may exhibit polymorphism, and that the disclosure encompasses all such forms, or mixtures thereof, which possess (1) PARP and/or (2) PARP-1 inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of anyone of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

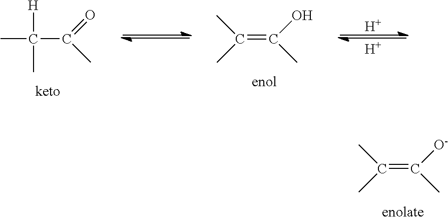

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidized to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, el al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77,285 (1988); f) N. Kakeya, et al., Chem. Pharm. Bull., 32,692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of anyone of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of anyone of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl C₁-C₄ alkylamine such as benzylamine and amino acids such as glycine or an ester thereof.

A suitable pharmaceutically acceptable prodrug of a compound of anyone of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tertbutyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesized by any one of the processes defined herein, the processes may then further comprise the additional steps of: (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula(I) can be isolated and purified using techniques well known in the art.

For example, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cycloheptylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutyl ketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis” 4th edition (2006) (John Wiley & Sons).

General routes for the preparation of a compound of the application are described in Schemes 1-12 herein.

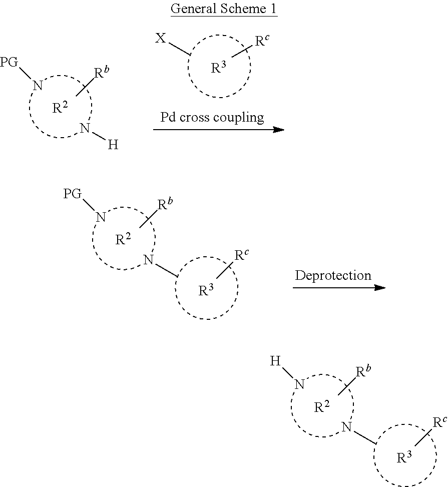

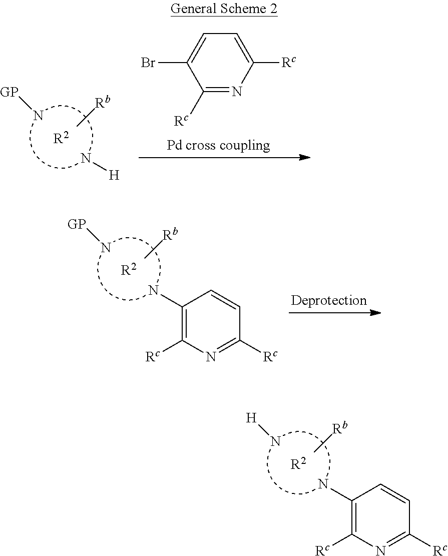

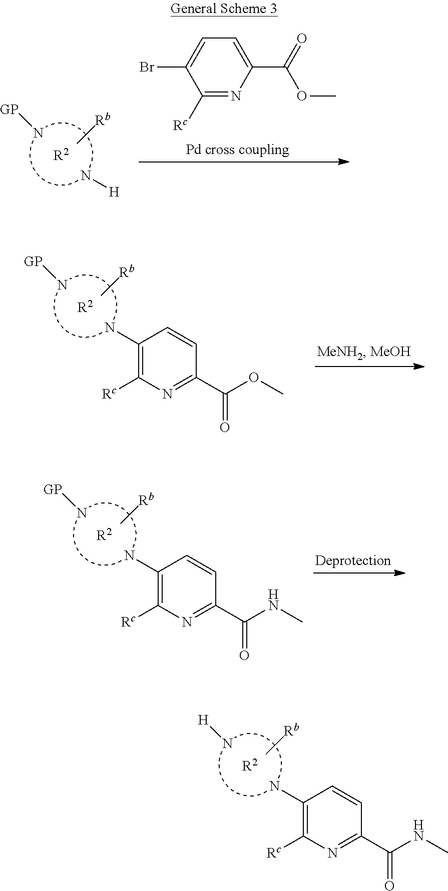

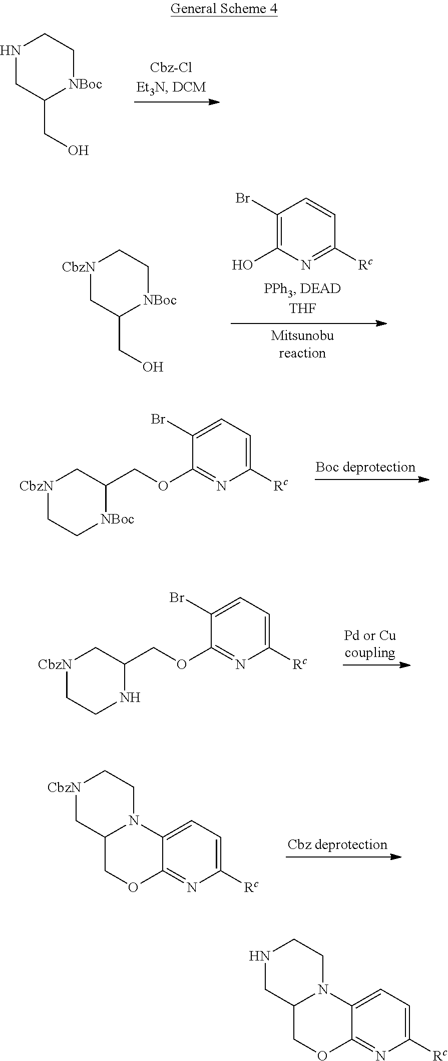

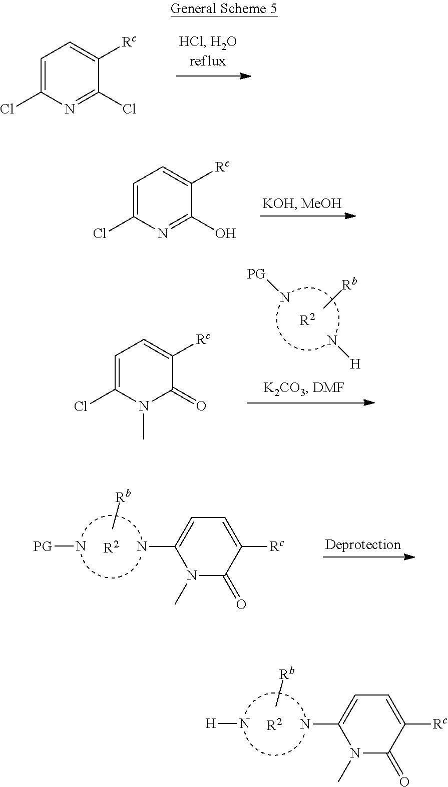

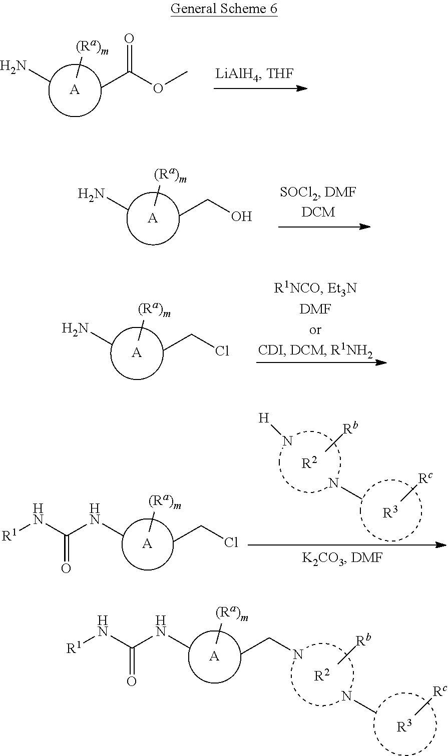

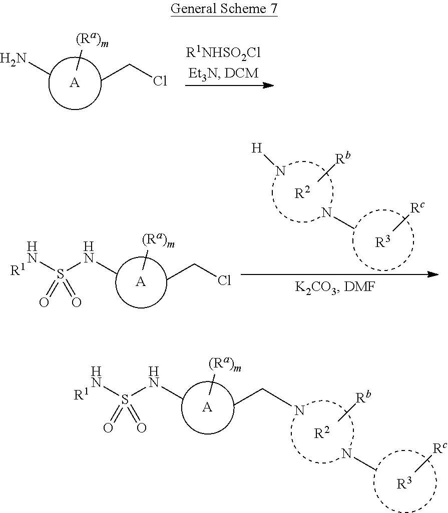

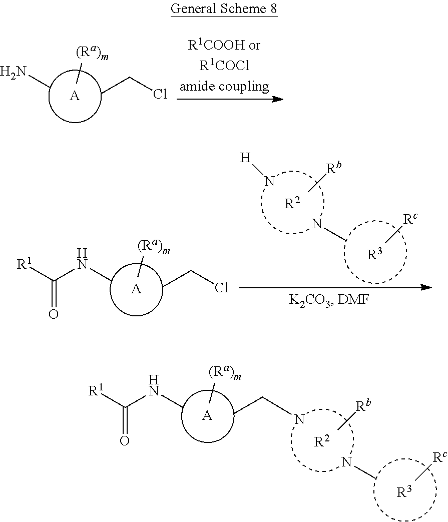

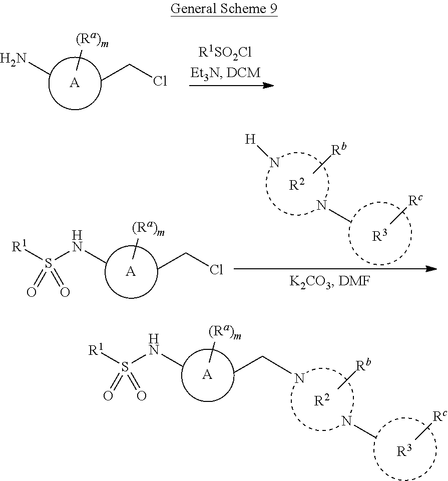

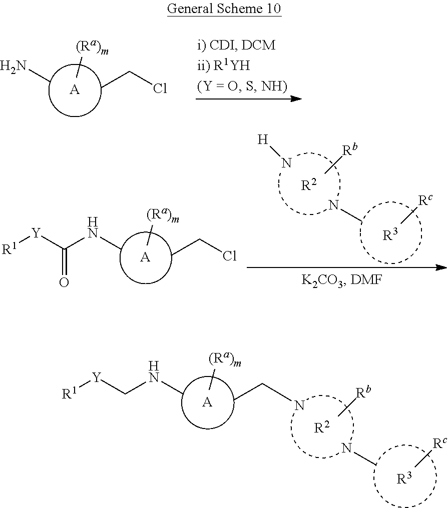

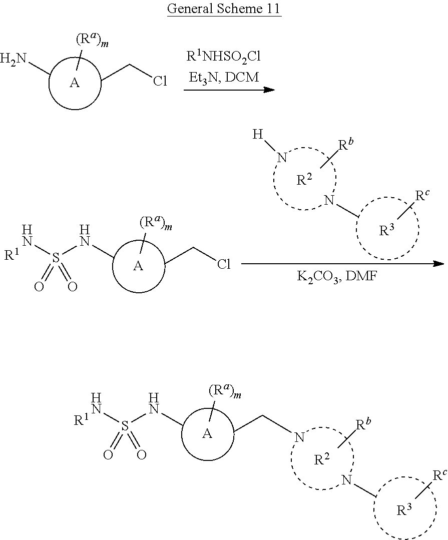

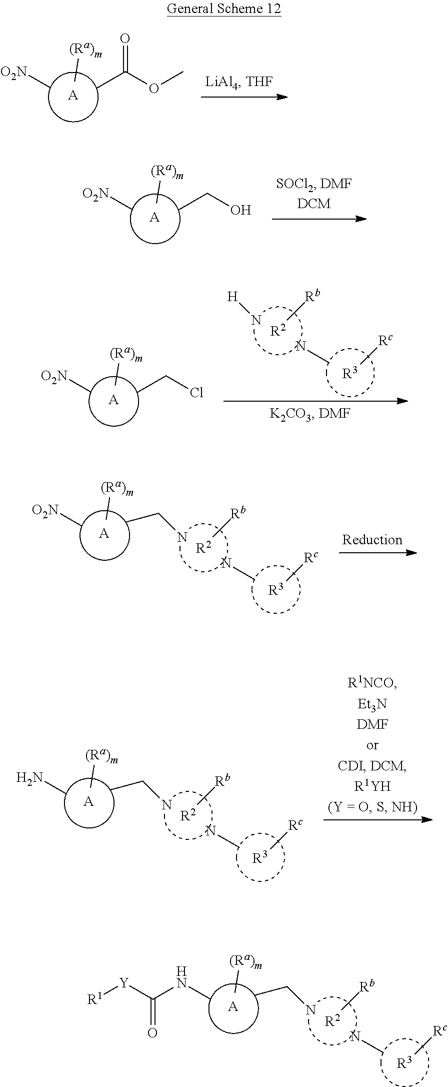

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein. In some embodiments, the biological assay measures PARP-1 binding by way of fluorescence polarization measurements. In some embodiments, binding of a test compound to recombinant human PARP-1 reduces the amount of binding of a fluorescent probe, and consequently reduces the detected level of fluorescence polarization. In some test compound stock solutions are prepared in a solvent, e.g., DMSO, and are serially diluted into a range of concentrations, e.g., 15 concentrations, 14 concentrations, 13 concentrations, 12 concentrations, 11 concentrations, 10 concentrations, 9 concentrations, 8 concentrations, 7 concentrations, 6 concentrations, or 5 concentrations, by a dilution factor, e.g., a 10-fold dilution factor, a 9-fold dilution factor, an 8-fold dilution factor, a 7-fold dilution factor, a 6-fold dilution factor, a 5-fold dilution factor, a 4-fold dilution factor, a 3-fold dilution factor, or a 2-fold dilution factor. In some embodiments, the serial dilutions, e.g., 100 nL of serial dilution, 90 nL of serial dilution, 80 nL of serial dilution, 70 nL of serial dilution, 60 nL of serial dilution, 50 nL of serial dilution, 30 nL of serial dilution, 20 nL of serial dilution, or 10 nL of serial dilution, are added to a multi-welled plate, e.g., a 1536-well plate, a 384-well plate, or a 96-well plate. In some embodiments, PARP-1, e.g., GST-tagged PARP-1, is diluted in assay buffer, e.g., 50 μL of assay buffer, 40 μL of assay buffer, e.g., 30 μL of assay buffer, 20 μL of assay buffer, 10 μL of assay buffer, or 5 μL of assay buffer, and is added to the plate. In some embodiments, the plate is centrifuged, e.g., at 1000 rpm for 1 min, and incubated, e.g., incubated for 30 minutes at room temperature (RT). In some embodiments, a fluorescent probe, e.g., 10 μL of 6 nM PARP1-FL (TOCRIS, Cat #6461), is diluted in an assay buffer and is added to the plate. In some embodiments, the final concentrations of PARP-1 and fluorescent probe are 20 nM and 3 nM, respectively, in a total volume of 20 μL. In some embodiments, the plate is centrifuged, e.g., at 1000 rpm for 1 minute, and the assay plate is incubated, e.g., for 4 h at RT. In some embodiments, the samples in each well are read using a plate reader, e.g., an Envision instrument using excitation=480 nm, and emission wavelength=FITC FP-P pol 535 nm & FITC FP-S pol 535 nm). In some embodiments, percent inhibition is calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. In some embodiments, binding IC₅₀ values are calculated using XLFit (equation 201: y=A+((B−A)/(1+((x/C)\hat{ }D))), where A=bottom, B=top, C=IC₅₀, and D=slope) with a floating top and bottom for curves.

In some embodiments, the biological assay measures PARP-2 binding by way of fluorescence polarization measurements. In some embodiments, binding of a test compound to recombinant human PARP-2 reduces the amount of binding of a fluorescent probe, and consequently reduces the detected level of fluorescence polarization. In some test compound stock solutions are prepared in a solvent, e.g., DMSO, and are serially diluted into a range of concentrations, e.g., 15 concentrations, 14 concentrations, 13 concentrations, 12 concentrations, 11 concentrations, 10 concentrations, 9 concentrations, 8 concentrations, 7 concentrations, 6 concentrations, or 5 concentrations, by a dilution factor, e.g., a 10-fold dilution factor, a 9-fold dilution factor, an 8-fold dilution factor, a 7-fold dilution factor, a 6-fold dilution factor, a 5-fold dilution factor, a 4-fold dilution factor, a 3-fold dilution factor, or a 2-fold dilution factor. In some embodiments, the serial dilutions, e.g., 100 nL of serial dilution, 90 nL of serial dilution, 80 nL of serial dilution, 70 nL of serial dilution, 60 nL of serial dilution, 50 nL of serial dilution, 30 nL of serial dilution, 20 nL of serial dilution, or 10 nL of serial dilution, are added to a multi-welled plate, e.g., a 1536-well plate, a 384-well plate, or a 96-well plate. In some embodiments, PARP-2, e.g., GST-tagged PARP-2, is diluted in assay buffer, e.g., 50 μL of assay buffer, 40 μL of assay buffer, e.g., 30 μL of assay buffer, 20 μL of assay buffer, 10 μL of assay buffer, or 5 μL of assay buffer, and is added to the plate. In some embodiments, the plate is centrifuged, e.g., at 1000 rpm for 1 min, and incubated, e.g., incubated for 30 minutes at room temperature (RT). In some embodiments, a fluorescent probe, e.g., 10 μL of 6 nM PARP1-FL (TOCRIS, Cat #6461), is diluted in an assay buffer and is added to the plate. In some embodiments, the final concentrations of PARP-2 and fluorescent probe are 20 nM and 3 nM, respectively, in a total volume of 20 PL. In some embodiments, the plate is centrifuged, e.g., at 1000 rpm for 1 minute, and the assay plate is incubated, e.g., for 4 h at RT. In some embodiments, the samples in each well are read using a plate reader, e.g., an Envision instrument using excitation=480 nm, and emission wavelength=FITC FP-P pol 535 nm & FITC FP-S pol 535 nm). In some embodiments, percent inhibition is calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. In some embodiments, binding IC₅₀ values are calculated using XLFit (equation 201. y=A+((B−A)/(1+((x/C)\hat{ }D))), where A=bottom, B=top, C=IC₅₀, and D=slope) with a floating top and bottom for curves.

In some embodiments, the biological assay measures the effects that compounds of the present disclosure may have on the viability of cells, e.g., the viability of cancer cells. In some embodiments, DLD-1 wild-type and DLD-1 BRCA2(−/−) colorectal adenocarcinoma cells, an isogenic pair of cell lines differing in the presence and absence, respectively, of both BRCA2 alleles, are used to measure the effect of the disclosed compounds on the viability of cancer cells. In some embodiments, the cells are harvested during the logarithmic growth period, counted, and seeded at a specified count in the wells of a 96- or 384-well cell culture plate. In some embodiments, after seeding, the cells are incubated, e.g., at 37° C., 5% CO₂ overnight. In some embodiments, the cells are treated with compounds, e.g., with serially diluted test compounds at 8-11 concentrations within a desired concentration range, e.g., from 1 nM-10 μM, for the generation of dose-response curves. In some embodiments, the plate is further incubated, e.g., for another 3-7 days in a humidified incubator at 37° C. and 5% CO₂. In some embodiments, cell viability is assessed by luminescence measurement after addition of Cell Titer-Glo reagent (Promega, Madison, Wis.) according to the manufacturer's instructions. In some embodiments, cell viability IC₅₀ values are calculated using XLFit, equation 201: y=A+((B−A)/(1+((x/C)\hat{ }D))), where A=bottom, B=top, C=IC₅₀, and D=slope. In some embodiments, the effects of the test compounds on the viability of other cell lines such as MDA-MB-436, MDA-MB-231, SUM149PT, HCC1395, and UWB1.289 are determined in an analogous method.

### Pharmaceutical Compositions

In some embodiments, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-s-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β—CD), maltosyl-β-cyclodextrin, γ-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an (1) PARP and/or (2) PARP-1 related condition referred to herein, slow its progression and/or reduce the symptoms associated with the condition.

An effective amount of a compound of the present disclosure for use in therapy is an amount sufficient to treat an (1) PARP and/or (2) PARP-1 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

Homologous recombination (HR) is a process critical to the repair of DNA double-strand breaks (DSBs) that, when defective, leads to the accumulation of DNA damage and genomic instability (Mekonnen N, et al. Frontiers in oncology. 2022:2747). Defective or deficient HR can result from mutation, loss of function, or inactivation of one or more genes involved in HR.

BRCA1 and BRCA2 are important tumor suppressor genes with a central role in the repair of DNA DSBs by HR. Deleterious mutations in BRCA1 and/or BRCA2 result in HR deficiency and are linked to increased risk of several cancers, most notably breast and ovarian cancer. Other genes involved in HR-mediated repair and whose mutation, loss of function, or inactivation can contribute to defects in HR include members of the FANC gene family, ATM, RAD51, PALB2, MRE11A, RAD50, NBS1, and EMSY.

Some sporadic cancers do not carry BRCA1 or BRCA2 mutations but display a BRCAness phenotype. Cancers with a BRCAness phenotype are cancers that share phenotypic characteristics of cancers that have germline BRCA1 or BRCA2 mutations, particularly HR deficiency (Turner N, et al. Nature reviews cancer. 2004 October; 4(10):814-9). The HR deficiency may result from inactivation of tumor suppressor genes including HR repair genes by mutation, loss of heterozygosity, or promoter hypermethylation. Phenotypic characteristics of cancers with germline BRCA1 or BRCA2 mutations are generally associated with genomic instability and include both chromosomal and sub-chromosomal aberrations such as mutations, structural copy number changes, and/or structural rearrangements (Mekonnen N, et al., 2022).

PARP inhibition has been found to be synthetically lethal to cancers with mutations in BRCA1 and/or BRCA2, both preclinically and clinically (Lord C J, et al. Science. 2017 Mar. 17; 355(6330):1152-8). Cancers with a BRCAness phenotype are also thought to be particularly susceptible to PARP inhibition. PARP inhibitors are part of standard-of-care treatments for cancers with defects in HR repair of DNA. These cancers include HR-deficient breast, ovarian, prostate and pancreatic cancers.

In some embodiments, the present disclosure provides a method of modulating PARP1 activity (e.g., in vitro or in vivo), comprising contacting a cell with an effective amount of a compound of the present disclosure or a pharmaceutically acceptable salt thereof.

In some embodiments, the present disclosure provides a method of modulating PARP1 activity (e.g., in vitro or in vivo), comprising contacting a cell with a compound of the present disclosure or a pharmaceutically acceptable salt thereof.

In some embodiments, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some embodiments, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating or preventing a disease or disorder disclosed herein in a subject in need thereof, comprising administering to the subject a compound of the present disclosure or a pharmaceutically acceptable salt thereof.

In some embodiments, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated PARP1 activity. In some embodiments, the disease or disorder is a disease or disorder in which PARP1 activity is implicated.

In some embodiments, the disease or disorder is cancer.

In some embodiments, the disease or disorder is an HR-deficient cancer.

In some embodiments, the disease or disorder is a BRCA1- or BRCA2-mutated cancer.

In some embodiments, the present disclosure provides a method of treating or preventing cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating or preventing HR-deficient cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating or preventing BRCA1- or BRCA2-mutated cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating HR-deficient cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating BRCA1- or BRCA2-mutated cancer in a subject in need thereof, comprising administering to the subject a therapeutically effective amount of a compound of the present disclosure or a pharmaceutically acceptable salt thereof, or a pharmaceutical composition of the present disclosure.

In some embodiments, the present disclosure provides a method of treating or preventing cancer in a subject in need thereof, comprising administering to the subject a compound of the present disclosure or a pharmaceutically acceptable salt thereof, or a pharmaceutical composition of the present disclosure.

In some embodiments, the present disclosure provides a method of treating or preventing HR-deficient cancer in a subject in need thereof, comprising administering to the subject a compound of the present disclosure or a pharmaceutically acceptable salt thereof, or a pharmaceutical composition of the present disclosure.

In some embodiments, the present disclosure provides a method of treating or preventing BRCA1- or BRCA2-mutated cancer in a subject in need thereof, comprising administering to the subject a compound of the present disclosure or a pharmaceutically acceptable salt thereof, or a pharmaceutical composition of the present disclosure.

In some embodiments, the present disclosure provides a method of treating cancer in a subject in need thereof, comprising administering to the subject a compound of the present disclosure or a pharmaceutically acceptable salt thereof, or a pharmaceutical composition of the present disclosure.

In some embodiments, the present disclosure provides a method of treating HR-deficient cancer in a subject in need thereof, comprising administering to the subject a compound of the present disclosure or a pharmaceutically acceptable salt thereof, or a pharmaceutical composition of the present disclosure.

In some embodiments, the present disclosure provides a method of treating BRCA1- or BRCA2-mutated cancer in a subject in need thereof, comprising administering to the subject a compound of the present disclosure or a pharmaceutically acceptable salt thereof, or a pharmaceutical composition of the present disclosure.

In some embodiments, the present disclosure provides a compound of the present disclosure or a pharmaceutically acceptable salt thereof for use in modulating PARP1 activity (e.g., in vitro or in vivo).

In some embodiments, the present disclosure provides a compound of the present disclosure or a pharmaceutically acceptable salt thereof for use in treating or preventing a disease or disorder disclosed herein.

In some embodiments, the present disclosure provides a compound of the present disclosure or a pharmaceutically acceptable salt thereof for use in treating a disease or disorder disclosed herein.

In some embodiments, the present disclosure provides a compound of the present disclosure or a pharmaceutically acceptable salt thereof for use in treating or preventing cancer in a subject in need thereof.

In some embodiments, the present disclosure provides a compound of the present disclosure or a pharmaceutically acceptable salt thereof for use in treating or preventing HR-deficient cancer in a subject in need thereof.

In some embodiments, the present disclosure provides a compound of the present disclosure or a pharmaceutically acceptable salt thereof for use in treating or preventing BRCA1- or BRCA2-mutated cancer in a subject in need thereof.

In some embodiments, the present disclosure provides a compound of the present disclosure or a pharmaceutically acceptable salt thereof for use in treating cancer in a subject in need thereof.

In some embodiments, the present disclosure provides a compound of the present disclosure or a pharmaceutically acceptable salt thereof for use in treating HR-deficient cancer in a subject in need thereof.

In some embodiments, the present disclosure provides a compound of the present disclosure or a pharmaceutically acceptable salt thereof for use in treating BRCA1- or BRCA2-mutated cancer in a subject in need thereof.

In some embodiments, the present disclosure provides use of a compound of the present disclosure or a pharmaceutically acceptable salt thereof in the manufacture of a medicament for modulating PARP1 activity (e.g., in vitro or in vivo).

In some embodiments, the present disclosure provides use of a compound of the present disclosure or a pharmaceutically acceptable salt thereof in the manufacture of a medicament for treating or preventing a disease or disorder disclosed herein.

In some embodiments, the present disclosure provides use of a compound of the present disclosure or a pharmaceutically acceptable salt thereof in the manufacture of a medicament for treating a disease or disorder disclosed herein.

In some embodiments, the present disclosure provides use of a compound of the present disclosure or a pharmaceutically acceptable salt thereof in the manufacture of a medicament for treating or preventing cancer in a subject in need thereof.

In some embodiments, the present disclosure provides use of a compound of the present disclosure or a pharmaceutically acceptable salt thereof in the manufacture of a medicament for treating or preventing HR-deficient cancer in a subject in need thereof.

In some embodiments, the present disclosure provides use of a compound of the present disclosure or a pharmaceutically acceptable salt thereof in the manufacture of a medicament for treating or preventing BRCA1- or BRCA2-mutated cancer in a subject in need thereof.

In some embodiments, the present disclosure provides use of a compound of the present disclosure or a pharmaceutically acceptable salt thereof in the manufacture of a medicament for treating cancer in a subject in need thereof.

In some embodiments, the present disclosure provides use of a compound of the present disclosure or a pharmaceutically acceptable salt thereof in the manufacture of a medicament for treating HR-deficient cancer in a subject in need thereof.

In some embodiments, the present disclosure provides use of a compound of the present disclosure or a pharmaceutically acceptable salt thereof in the manufacture of a medicament for treating BRCA1- or BRCA2-mutated cancer in a subject in need thereof.

The present disclosure provides compounds that function as modulators of PARP1 activity.

In some embodiments, the compounds of the present disclosure are inhibitors of PARP1.

In some embodiments, the modulation of PARP1 is inhibition of PARP1.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PARP1 activity is implicated in a patient in need of such treatment, said method comprising administering to said patient a therapeutically effective amount of a compound, or a pharmaceutically acceptable salt thereof, or a pharmaceutical composition as defined herein.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which PARP1 activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardization of in vitro and in vivo test systems for the evaluation of the effects of modulators of PARP1 activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.): transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Examples

It is understood that the neutral compounds of Formula (I) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

### Abbreviations

¹H Proton
° C. Degree centigrade
ACN/CH₃CN Acetonitrile
AgF₂ Silver (II) fluoride
Aq. Aqueous
br s Broad singlet
BOc₂O Di-tert-butyl dicarbonate
Brine Saturated sodium chloride solution
CDI 1,1′-Carbonyldiimidazole
Cs₂CO₃ Cesium carbonate
CuBr Copper (1) bromide
d Doublet
dd Doublet of doublets
DCM Dichloromethane
DLPEA N,N-diisopropylethylamine
DMF N,N-dimethylformamide
DMAP 4-Dimethylaminopyridine
EDCI 1-Ethyl-3-(3-dimethylaminopropyl)carbodiimide
EtMgBr Ethylmagnesium bromide
EtOAc Ethyl acetate
Et₃N Triethylamine
EtNCO Ethyl isocyanate
FA Formic acid
h Hour
HATU 1-[Bis(dimethylamino)methylene]-1H-1,2,3-triazolo[4,5-b]pyridinium 3-oxid hexafluorophosphate
HCl Hydrochloric acid
HCOOH Formic acid
HPLC High-Performance Liquid Chromatography
iPrOH Isopropanol
J Coupling constant
K₂CO₃ Potassium carbonate
KI Potassium iodide
K₃PO₄ Tripotassium phosphate
LCMS Liquid chromatography mass spectrometry
LiAlH₄ Lithium aluminium hydride
LiBH₄ Lithium borohydride
MeNH₂ Methylamine
MeOH Methanol
m Multiplet
m/z Mass/charge ratio
NaOH Sodium hydroxide
Na₂SO₄ Sodium sulfate
NH₂CN Cyanamide
NH₄Cl Ammonium chloride
NMR Nuclear magnetic resonance
Prep Preparative
Pd(OAc)₂ Palladium acetate
Pd₂(dba)₃ Tris(dibenzylideneacetone)dipalladium
q Quartet
RT Room temperature
s Singlet
SOCl₂ Thionyl chloride
t Triplet
TBDMSCl Tert-butyldimethylsilyl chloride
TEA/Et₃N Triethylamine
TFA Trifluoroacetic acid
THF Tetrahydrofuran

### Example S-1: Synthesis of 5-(4-((2-(3-ethylureido)pyridin-4-yl)methyl)piperazin-1-yl)-N,6-dimelhylpicolinamide formate (Compound 13)

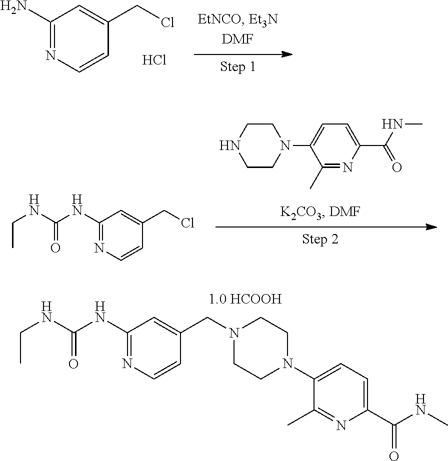 [SMILES: Nc1cc(CCl)ccn1 ; Cl ; CNC(=O)c1ccc(N2CCNCC2)c(C)n1 ; CCNC(=O)Nc1cc(CCl)ccn1 ; CCNC(=O)Nc1cc(CN2CCN(c3ccc(C(=O)NC)nc3C)CC2)ccn1]

Step 1: Synthesis of 1-(4-(chloromethyl)pyridin-2-yl)-3-ethylurea. To a stirred solution of 4-(chloromethyl)pyridin-2-amine hydrochloride (150 mg, 0.84 mmol) in DMF (1 mL) was added ethyl isocyanate (0.08 mL, 1.01 mmol) and Et₃N (0.12 mL, 0.84 mmol). The reaction was stirred at 25° C. for 16 h before it was quenched with aqueous NH₄Cl and extracted with EtOAc (2 mL×2). The combined organic layers were washed with brine, dried over Na₂SO₄, filtered and the filtrate was concentrated under reduced pressure. Purification by silica gel chromatography afforded the crude title compound (70 mg). LCMS (m/z): 214.1 [M+H]⁺.

Step 2: Synthesis of 5-(4-((2-(3-ethylureido)pyridin-4-yl)methyl)piperazin-1-yl)-N,6-dimethylpicolinamide formate. To a stirred solution of crude 1-(4-(chloromethyl) pyridin-2-yl)-3-ethylurea (70 mg) in DMF (1 mL) were added N,6-dimethyl-5-(piperazin-1-yl)picolinamide (77 mg, 0.33 mmol), K₂CO₃ (91 mg, 0.66 mmol) and the reaction mixture was warmed to 100° C. and stirred at that temperature for 4 h. The reaction mixture was quenched with aqueous NH₄Cl, and extracted with EtOAc (2 mL×2). The combined organic layers were washed with brine, dried over Na₂SO₄, filtered, and the filtrate was concentrated under reduced pressure. Purification by Prep-HPLC afforded the title compound (6.4 mg, 1.7%). LCMS (m/z): 412.3 [M+H]⁺. ¹H NMR (400 MHz, CD₃OD) δ 8.35 (s, 1H), 8.17 (d, J=5.3 Hz, 1H), 7.88 (d, J=8.3 Hz, 1H), 7.51 (d, J=8.3 Hz, 1H), 7.14 (s, 1H), 7.02 (d, J=5.3 Hz, 1H), 3.62 (s, 2H), 3.38-3.34 (m, 2H), 3.10-3.01 (m, 4H), 2.96 (s, 3H), 2.75-2.65 (m, 4H), 2.56 (s, 3H), 1.23 (t, J=7.2 Hz, 3H).

### Example S-2: Synthesis of 5-(4-((2-blutramidopyridin-4-yl)methyl)piperazin-yl)-N,6-dimethylpicolinamide (Compound 161)

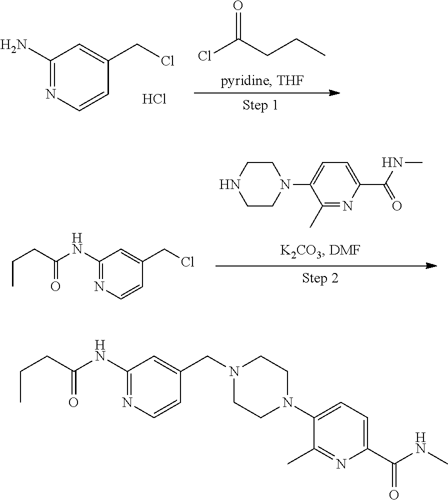

Step 1: Synthesis of N-(4-(chloromethyl)pyridin-2-yl)butyramide. To a suspension of 4-(chloromethyl)pyridin-2-amine hydrochloride (200 mg, 1.12 mmol) and pyridine (177 mg, 2.24 mmol) in THF (5 mL) at 0° C. was added butyryl chloride (119 mg, 1.12 mmol) and the reaction mixture was stirred at the same temperature for 1 h. The reaction was quenched with saturated aqueous NaHCO₃ and extracted with DCM. The organic layer was concentrated in vacuo. The residue was purified by silica gel chromatography to afford the title compound (25 mg, 10⁰/). LCMS (m/z): 213.2 [M+H]⁺.

Step 2: Synthesis of 5-(4-((2-butyramidopyridin-4-yl)methyl)piperazin-1-y)-N,6-dimethylpicolinamide. To a solution of N-[5-(chloromethyl)pyridin-2-yl]butanamide (25 mg, 0.12 mmol) in DMF (1 mL) were added N,6-dimethyl-5-(piperazin-1-yl)picolinamide (28 mg, 0.12 mmol) and potassium carbonate (50 mg, 0.36 mmol). The resulting solution was stirred at 60° C. for 15 h before it was concentrated. The residue was purified by Prep-HPLC to afford the title compound (9 mg, 18%). LCMS (m/z): 411.3 [M+H]⁺. ¹H NMR (400 MHz, CD₃OD) δ 8.22 (d, J=4.7 Hz, 1H), 8.14 (s, 1H), 7.84 (d, J=8.3 Hz, 1H), 7.47 (d, J=8.3 Hz, 1H), 7.14 (d, J=5.0 Hz, 1H), 3.69 (d, J=8.8 Hz, 2H), 3.08-3.00 (m, 4H), 2.91 (s, 3H), 2.80-2.68 (m, 4H), 2.52 (s, 3H), 2.39 (t, J=7.4 Hz, 2H), 1.77-1.65 (m, 2H), 0.99 (t, J=7.4 Hz, 3H).

### Example S-3: Synthesis of N-methyl-3-(piperazin-1-yl)-1, %-naphthyridin-8-amine

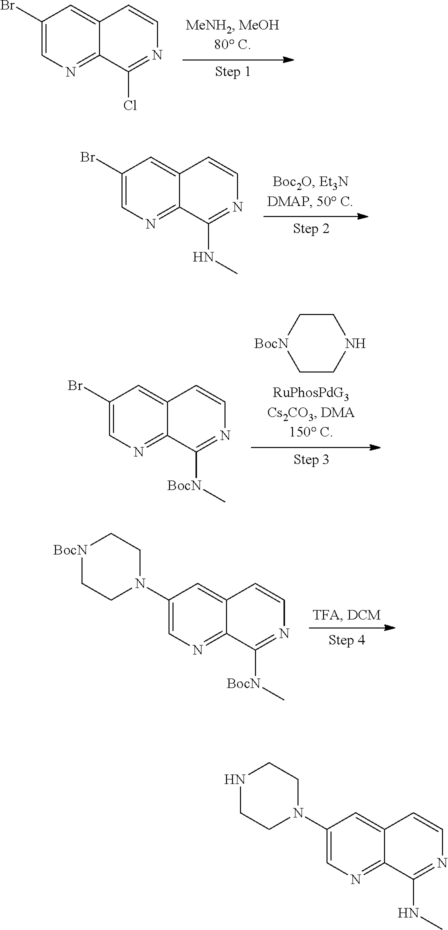 [SMILES: Clc1nccc2cc(Br)cnc12 ; CNc1nccc2cc(Br)cnc12 ; CC(C)(C)OC(=O)N1CCNCC1 ; CN(C(=O)OC(C)(C)C)c1nccc2cc(Br)cnc12 ; CN(C(=O)OC(C)(C)C)c1nccc2cc(N3CCN(C(=O)OC(C)(C)C)CC3)cnc12 ; CNc1nccc2cc(N3CCNCC3)cnc12]

Step 1: Synthesis of 3-bromo-N-methyl-1,7-naphthyridin-8-amine. To a solution of 3-bromo-8-chloro-1,7-naphthyridine (500 mg, 2.05 mmol) in MeOH (5 mL) was added methylamine (33% in methanol, 4 mL) and the reaction was stirred at 80° C. for 12 h before it was concentrated under reduced pressure and purified by silica gel chromatography to afford the title compound (450 mg, 92%). LCMS (m/z): 238.1 [M+H]⁺.

Step 2: Synthesis of tert-butyl (3-bromo-1,7-naphthyridin-8-yl) (methyl)carbamate. A mixture of 3-bromo-N-methyl-1,7-naphthyridin-8-amine (400 mg, 1.68 mmol), di-tert-butyl dicarbonate (3.67 g, 16.8 mmol), Et₃N (340 mg, 3.36 mmol) and DMAP (21 mg, 0.17 mmol) was stirred at 50° C. for 48 h. The reaction mixture was concentrated under reduced pressure and purified by silica gel chromatography to furnish the title compound (450 mg, 79%). LCMS (m/z): 338.0 [M+H]⁺.

Step 3: Synthesis of tert-butyl 4-(8-((tert-butoxycarbonyl)(methyl)amino)-1,7-naphthyridin-3-yl)piperazine-1-carboxylate. To a solution of tert-butyl (3-bromo-1,7-naphthyridin-8-yl)(methyl)carbamate (360 mg, 1.06 mmol) in DMA (4 mL) was added tert-butyl piperazine-1-carboxylate (197 mg, 1.06 mmol), Cs₂CO₃ (691 mg, 2.12 mmol), RuPhosPdC (89 mg, 0.11 mmol) and the reaction mixture was stirred at 150° C. for 2 h under nitrogen atmosphere. The reaction mixture was concentrated under reduced pressure and purified by silica gel chromatography to afford the title compound (290 mg, 62%). LCMS (m/z): 444.3[M+H]⁺.

Step 4: Synthesis of N-methyl-3-(piperazin-1-yl)-1,7-naphthyridin-8-amine. To a solution of tert-butyl-4-(8-((tert-butoxycarbonyl)(methyl)amino)-1,7-naphthyridin-3-yl) piperazine-1-carboxylate (290 mg, 0.65 mmol) in DCM (3 mL) was added TFA (1 mL) and the reaction mixture was stirred at 20° C. for 1 h before it was quenched with saturated NaHCO₃ solution, extracted with MeOH/DCM (1/10). The combined organic layers were washed with brine, dried over Na₂SO₄, filtered and the filtrate was concentrated to afford the title compound (140 mg, 88%). LCMS (m/z): 244.1 [M+H]⁺.

### Example S-4: Synthesis of 1-ethyl-3-(4-((4-(8-(methylamino)-1,7-naphthyridin-3-yl)piperazin-1-yl)methyl)pyridin-2-yl)urea (Compound 257)

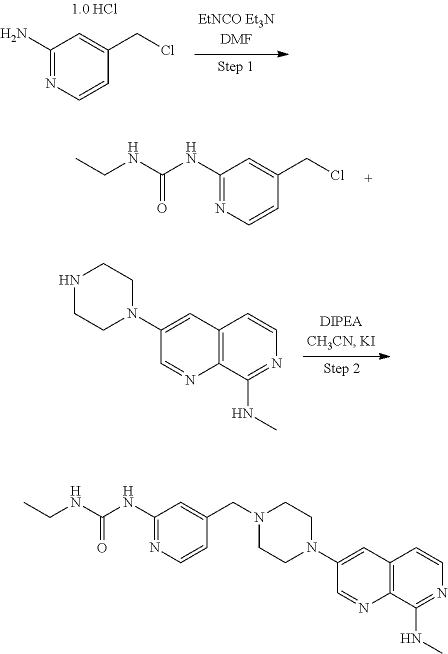 [SMILES: Nc1cc(CCl)ccn1 ; CCNC(=O)Nc1cc(CCl)ccn1 ; CNc1nccc2cc(N3CCNCC3)cnc12 ; CCNC(=O)Nc1cc(CN2CCN(c3cnc4c(NC)nccc4c3)CC2)ccn1]

Step 1: Synthesis of 1-(4-(chloromethyl)pyridin-2-yl)-3-ethylurea: To a stirred solution of 4-(chloromethyl)pyridin-2-amine hydrochloride (5 g, 27.9 mmol) in DMF (25 mL) were added ethyl isocyanate (4 g, 55.8 mmol) and Et₃N (5.6 g, 55.8 mmol) and the reaction mixture was stirred at 50° C. for 16 h before it was quenched with aqueous NH₄Cl, and extracted with EtOAc. The combined organic layers were washed with brine, dried over Na₂SO₄, and filtered. The filtrate was concentrated under reduced pressure and the residue was purified by silica gel chromatography to afford the title compound (1.2 g, 20%). LCMS (m/z): 214.1 [M+H]⁺.

Step 2: Synthesis of 1-ethyl-3-(4-((4-(8-(methylamino)-1,7-naphthyridin-3-yl)piperazin-1-yl)methyl)pyridin-2-yl)urea: A mixture of N-methyl-3-(piperazin-1-yl)-1,7-naphthyridin-8-amine (50 mg, 0.21 mmol), 1-[4-(chloromethyl)pyridin-2-yl]-3-ethylurea (44 mg, 0.21 mmol), KI (68 mg, 0.41 mmol) and DIPEA (53 mg, 0.41 mmol) in CH₃CN (3 mL) was stirred at 20° C. for 16 h. The reaction mixture was concentrated under reduced pressure and purified by silica gel chromatography to afford crude product, which was purified by Prep-HPLC and SFC to afford the title compound (21 mg, 24%). LCMS (m/z): 421.3 [M+H]⁺; ¹HNMR (400 MHz, CD₃OD) δ 8.59 (d, J=2.6 Hz, 1H), 8.12 (d, J=5.2 Hz, 1H), 7.67 (d, J=6.0 Hz, 1H), 7.24 (d, J=2.5 Hz, 1H), 7.10 (s, 1H), 6.98 (d, J=5.1 Hz, 1H), 6.70 (d, J=6.1 Hz, 1H), 3.56 (s, 2H), 3.44-3.36 (m, 4H), 3.35-3.30 (q, J=7.2 Hz, 2H), 3.04 (s, 3H), 2.69-2.61 (m, 4H), 1.18 (t, J=7.2 Hz, 3H).

### Example S-5: Synthesis of 3-fluoro-N-methyl-4-(piperazin-1-yl)benzamide hydrochloride.

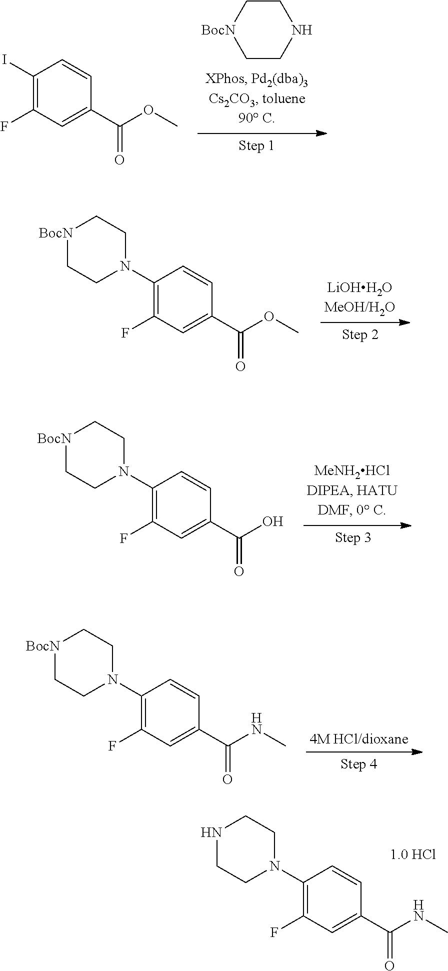

Step 1: Synthesis of tert-butyl 4-(2-fluoro-4-(methoxycarbonyl)phenyl)piperazine-1-carboxylate. A suspension of methyl 3-fluoro-4-iodobenzoate (1 g, 3.57 mmol), tert-butyl piperazine-1-carboxylate (738 mg, 3.94 mmol), XPhos (341 mg, 0.72 mmol), Pd₂(dba)₃ (328 mg, 0.36 mmol) and Cs₂CO₃ (2.3 g, 7.17 mmol) in toluene (80 mL) was stirred at 90° C. for 24 h under N2 atmosphere. The reaction mixture was concentrated under reduced pressure and purified by silica gel chromatography to afford the title compound (870 mg, 72%). LCMS (m/z): 339.0 [M+H]⁺.

Step 2: Synthesis of 4-(4-(tert-butoxycarbonyl)piperazin-1-yl)-3-fluorobenzoic acid. A solution of tert-butyl 4-(2-fluoro-4-(methoxycarbonyl)phenyl)piperazine-1-carboxylate (870 mg, 2.57 mmol) and lithium hydroxide monohydrate (322 mg, 7.69 mmol) in MeOH/H₂O (3:2, 100 mL) was stirred at 30° C. for 5 h. It was poured into water, extracted with TBME. The pH of the aqueous phase was adjusted to 2-3 and extracted with EtOAc. The organic layer was concentrated to furnish the title compound (700 mg, 84%). LCMS (m/z): 325.1 [M+H]⁺.

Step 3: Synthesis of tert-butyl 4-(2-fluoro-4-(methylcarbamoyl)phenyl)piperazine-1-carboxylate. A solution of 4-(4-(tert-butoxycarbonyl)piperazin-1-yl)-3-fluorobenzoic acid (650 mg, 2.0 mmol), methylamine hydrochloride (150 mg, 2.2 mmol), DIPEA (1.1 g, 8.8 mmol), HATU (759 mg, 2.0 mmol) in DMF (10 mL) was stirred at 0° C. for 1.5 h. The resulting solution was poured into water, extracted with EtOAc. The combined organic layers were concentrated, and the residue was purified by silica gel column chromatography to afford the title compound (560 mg, 83%). LCMS (m/z): 338.1 [M+H]⁺.

Step 4: Synthesis of 3-fluoro-N-methyl-4-(piperazin-1-yl)benzamide hydrochloride. A solution of tert-butyl 4-(2-fluoro-4-(methylcarbamoyl) phenyl)piperazine-1-carboxylate (560 mg, 1.7 mmol) in HCl/dioxane (4M, 30 mL) was stirred at 25° C. for 2 h. The reaction was concentrated to produce the title compound (3.27 g, crude). LCMS (m/z): 238.1 [M+H]⁺.

### Example S-6: Synthesis of 4-(4-((2-(3-ethylureido)pyridin-4-yl)methyl)piperazin-1-yl)-3-fluoro-N-methylbenzamide (Compound 31)

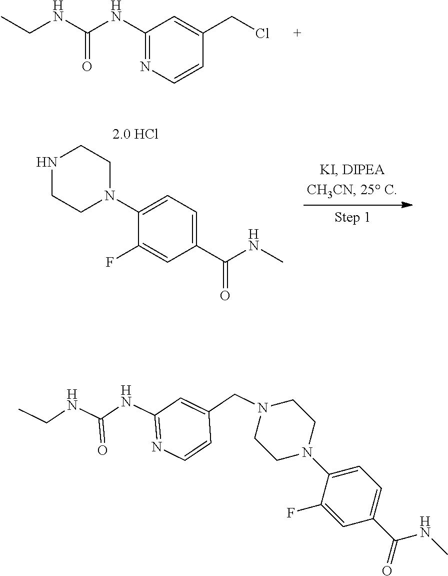 [SMILES: CCNC(=O)Nc1cc(CCl)ccn1 ; CNC(=O)c1ccc(N2CCNCC2)c(F)c1 ; CCNC(=O)Nc1cc(CN2CCN(c3ccc(C(=O)NC)cc3F)CC2)ccn1]

Step 1: Synthesis of 4-(4-((2-(3-ethylureido)pyridin-4-yl)methyl)piperazin-1-yl)-3-fluoro-N-methylbenzamide: A mixture of 1-(4-(chloromethyl)pyridin-2-yl)-3-ethylurea (60 mg, 0.28 mmol), 3-fluoro-N-methyl-4-(piperazin-1-yl)benzamide dihydrochloride (174 mg, 0.56 mmol), KI (70 mg, 0.42 mmol) and DIPEA (362 mg, 2.80 mmol) in CH₃CN (5 mL) was stirred at 25° C. for 16 h. The reaction mixture was concentrated under reduced pressure and the residue was purified by silica gel chromatography to obtain the crude product, which was purified by Prep-HPLC to afford the title compound (21 mg, 18%). LCMS (m/z): 415.3 [M+H]: ¹HNMR (400 MHz, DMSO-d₆) δ 9.12 (s, 1H), 8.35-8.30 (m, 1H), 8.21-8.15 (m, 1H), 8.11 (d, J=5.2 Hz, 1H), 7.63-7.54 (m, 2H), 7.34 (s, 1H), 7.08-7.03 (m, 1H), 6.91-6.88 (m, 1H), 3.50 (s, 2H), 3.22-3.14 (m, 2H), 3.14-3.08 (m, 4H), 2.75 (d, J=4.5 Hz, 3H), 2.57-2.52 (m, 4H), 1.09 (t, J=7.2 Hz, 3H).

### Example S-7: Synthesis of 1-(6-methyl-5-(piperazin-1-yl)pyridin-2-yl)propan-1-one hydrochloride

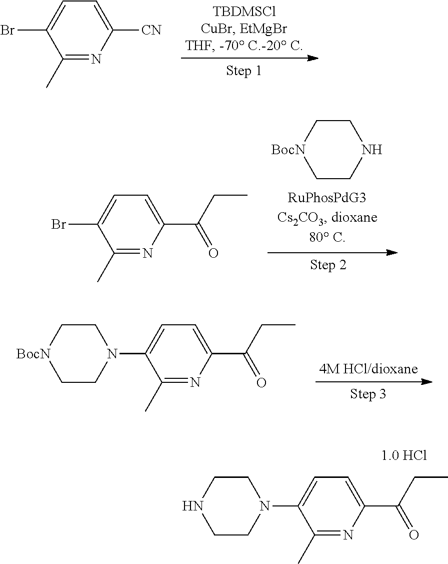

Step 1: Synthesis of 1-(5-bromo-6-methylpyridin-2-yl)propan-1-one. To a stirred solution of 5-bromo-6-methylpicolinonitrile (5 g, 25.4 mmol) in THF (50 mL) at −70° C. was added tert-butyldimethylsilyl chloride (3.83 g, 25.4 mmol), CuBr (0.11 g, 0.7 mmol), ethyl magnesium bromide (3M in Et₂O, 18.6 mL, 55.8 mmol) and the reaction mixture was stirred at 20° C. for 2.5 h before it was quenched with NH₄Cl and filtered. The resulting solution was poured into ice water, extracted with EtOAc. The combined organic layers were concentrated, and the residue was purified by silica gel column chromatography to afford the title compound (2.72 g, 47%). LCMS (m/z): 228.1 [M+H]⁺.

Step 2: Synthesis of tert-butyl 4-(2-methyl-6-propionylpyridin-3-yl)piperazine-1-carboxylate. A suspension of 1-(5-bromo-6-methylpyridin-2-yl)propan-1-one (2.67 g, 11.7 mmol), tert-butyl piperazine-1-carboxylate (3.26 g, 17.5 mmol), RuPhosPdG3 (0.98 g, 1.2 mmol), CS₂CO₃ (7.61 g, 23.4 mmol) in dioxane (40 mL) was stirred at 80° C. for 16 h under N2 atmosphere. The resulting solution was poured into ice water, extracted with EtOAc. The combined organic layers were concentrated, and the residue was purified by silica gel column chromatography to furnish the title compound (2.57 g, 66%). LCMS (m/z): 334.1 [M+H]⁺.

Step 3: Synthesis of 1-(6-methyl-5-(piperazin-1-yl)pyridin-2-yl)propan-1-one hydrochloride. A solution of tert-butyl 4-(2-methyl-6-propionylpyridin-3-yl)piperazine-i-carboxylate (2.57 g, 7.7 mmol) in 1,4-dioxane/HCl (4M, 30 mL) was stirred at 25° C. for 2 h. The reaction was concentrated to produce the title compound (3.27 g, crude). LCMS (m/z): 234.3 [M+H]r.

### Example S-8: Synthesis of 1-ethyl-3-(4-((4-(2-methyl-6-propionrylpyridin-3-yl)piperazin-1-yl)methyl)pyridin-2-yl)urea (Compound 7)

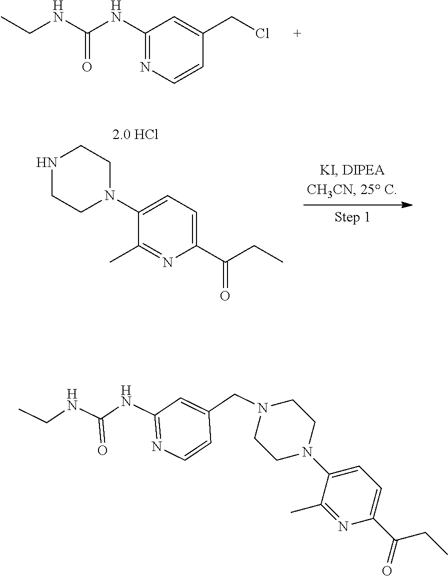 [SMILES: CCNC(=O)Nc1cc(CCl)ccn1 ; CCC(=O)c1ccc(N2CCNCC2)c(C)n1 ; CCNC(=O)Nc1cc(CN2CCN(c3ccc(C(=O)CC)nc3C)CC2)ccn1]

Step 1: Synthesis of 1-ethyl-3-(4-((4-(2-methyl-6-propionylpyridin-3-yl)piperazin-1-yl)methyl)pyridin-2-yl)urea: A mixture of 1-(4-(chloromethyl)pyridin-2-yl)-3-ethylurea (90 mg, 0.42 mmol), 1-(6-methyl-5-(piperazin-1-yl)pyridin-2-yl)propan-1-one dihydrochloride (257 mg, 0.84 mmol), KI (105 mg, 0.63 mmol) and DIPEA (543 mg, 4.2 mmol) in CH₃CN (4 mL) was stirred at 25° C. for 16 h. The reaction mixture was concentrated under reduced pressure and the residue was purified by silica gel chromatography to afford the crude product, which was purified by Prep-HPLC to afford the title compound (50 mg, 29%). LCMS (m/z): 411.3 [M+H]; ¹HNMR (400 MHz, DMSO-d₆) δ 9.10 (s, 1H), 8.16-8.11 (m, 1H), 8.07 (d, J=5.2 Hz, 1H), 7.73 (d, J=8.3 Hz, 1H), 7.43 (d, J=8.4 Hz, 1H), 7.30 (s, 1H), 6.87-6.84 (m, 1H), 3.48 (s, 2H), 3.18-3.10 (m, 2H), 3.10-3.03 (m, 2H), 2.98-2.92 (m, 4H), 2.57-2.49 (m, 4H), 2.45 (s, 3H), 1.06-0.99 (m, 6H).

### Example S-9: Synthesis of N,6-dimethyl-5-(piperazin-1-yl) picolinamide dihydrochloride

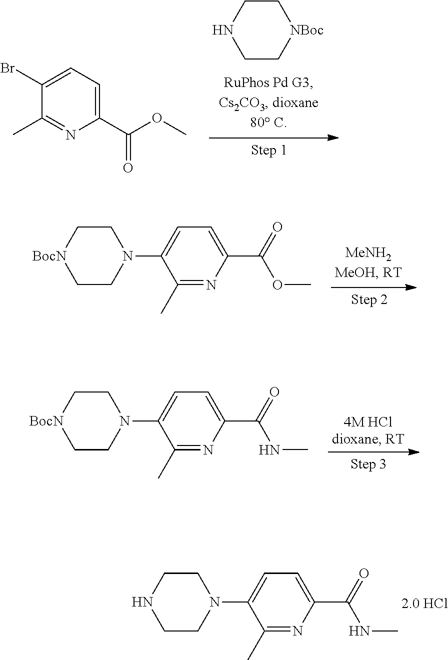 [SMILES: CC(C)(C)OC(=O)N1CCNCC1 ; COC(=O)c1ccc(Br)c(C)n1 ; COC(=O)c1ccc(N2CCN(C(=O)OC(C)(C)C)CC2)c(C)n1 ; CNC(=O)c1ccc(N2CCN(C(=O)OC(C)(C)C)CC2)c(C)n1 ; CNC(=O)c1ccc(N2CCNCC2)c(C)n1]

Step 1: Synthesis of tert-butyl 4-[6-(methoxycarbonyl)-2-methylpyridin-3-yl]piperazine-1 carboxylate: To a stirred solution of methyl 5-bromo-6-methylpyridine-2-carboxylate (10 g, 43.47 mmol) in dioxane (200 mL) were added tert-butyl piperazine-1-carboxylate (12.15 g, 65.23 mmol), RuPhosPdG3 (3.64 g, 4.35 mmol) and Cs₂CO₃ (28.35 g, 0.087 mol) and the reaction mixture was stirred at 80° C. for 18 h under N2 before it was quenched with aqueous NH₄Cl, extracted with EtOAc (200 mL×2). The combined organic layers were washed with brine, dried over Na₂SO₄, filtered and the filtrate was concentrated under reduced pressure. Purification by silica gel chromatography afforded the title compound (8.7 g, 60%). LCMS (m/z): 336.2 [M+H]⁺.

Step 2: Synthesis of tert-butyl 4-[2-methyl-6-(methyl carbamoyl) pyridin-3-yl]piperazine-1-carboxylate: To a stirred solution of tert-butyl 4-[6-(methoxycarbonyl)-2-methylpyridin-3-yl] piperazine-1-carboxylate (8.7 g, 25.94 mmol) in MeOH (20 mL) was added MeNH₂ (33% in MeOH, 100 mL) and the reaction mixture was stirred at RT for 18 h before it was concentrated under reduced pressure and purified by silica gel chromatography to afford the title compound (8 g, 92%). LCMS (m/z): 335.3 [M+H]⁺.

Step 3: Synthesis of N,6-dimethyl-5-(piperazin-1-yl) picolinamide dihydrochloride: To a stirred solution of tert-butyl 4-[2-methyl-6-(methyl carbamoyl) pyridin-3-yl] piperazine-1-carboxylate (8 g, 23.92 mmol) in DCM (60 mL) was added 4M HCl in dioxane (20 mL) and the reaction mixture was stirred at 25° C. for 4 h. The reaction mixture was concentrated under reduced pressure and the residue was purified by silica gel chromatography to afford the title compound (6.7 g, 91%). LCMS (m/z): 235.2 [M+H]⁺.

### Example S-10: Synthesis of 5-(4-((2-(3-isopropylureido)pyridin-4-yl)methyl)piperazin-1-yl)-N,6-dimethylpicolinamide (Compound 259)

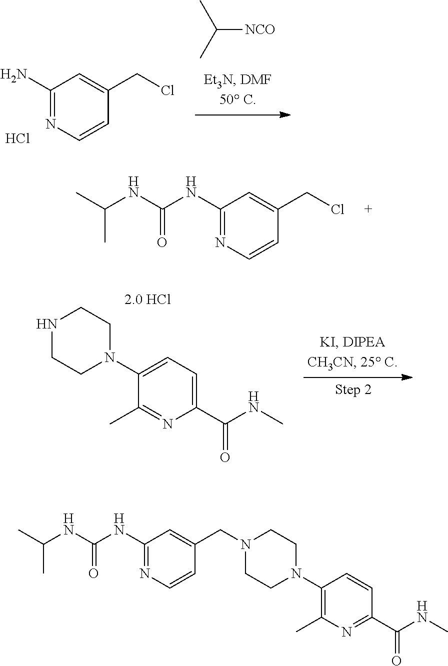

Step 1: Synthesis of 1-(4-(chloromethyl)pyridin-2-yl)-3-isopropylurea: To a stirred solution of 4-(chloromethyl)pyridin-2-amine hydrochloride (100 mg, 0.56 mmol) in DMF (3 mL) was added isopropyl isocyanate (57 mg, 0.67 mmol), Et₃N (113 mg, 1.12 mmol) and the reaction was stirred at 50° C. for 5 h. The reaction was quenched with aqueous NH₄Cl, and extracted with EtOAc (50 mL×2). The combined organic layers were washed with brine, dried over Na₂SO₄, filtered and the filtrate was concentrated under reduced pressure. The residue was purified by silica gel chromatography to afford the title compound (50 mg, 39%). LCMS (m/z): 228.2 [M+H]⁺.

Step 2: Synthesis of 5-(4-((2-(3-isopropylureido)pyridin-4-yl)methyl)piperazin-1-yl)-N,6-dimethylpicolinamide: A mixture of 1-(4-(chloromethyl)pyridin-2-yl)-3-isopropylurea (50 mg, 0.22 mmol), N,6-dimethyl-5-(piperazin-1-yl)picolinamide dihydrochloride (101 mg, 0.33 mmol), KI (55 mg, 0.33 mmol) and DIPEA (284 mg, 2.2 mmol) in CH₃CN (5 mL) was stirred at 25° C. for 16 h. The reaction mixture was concentrated under reduced pressure and the residue was purified by silica gel chromatography to afford crude product, which was purified by prep-HPLC to afford the title compound (33 mg, 35%). LCMS (m/z): 426.3 [M+H]⁺; ¹HNMR (400 MHz, DMSO-d₆) δ 9.04 (s, 1H), 8.47-8.39 (m, 1H), 8.11 (q, J=5.2 Hz, 1H), 8.01 (d, J=5.9 Hz, 1H), 7.80 (d, J=8.3 Hz, 1H), 7.48 (d, J=8.3 Hz, 1H), 7.38 (s, 1H), 6.90 (d, J=4.8 Hz, 1H), 3.88-3.75 (m, 1H), 3.52 (s, 2H), 3.01-2.89 (m, 4H), 2.80 (d, J=4.8 Hz, 3H), 3.01-2.89 (m, 4H), 2.49 (s, 3H), 1.13 (d, J=6.5 Hz, 6H).

### Example S-11: Synthesis of 5-(4-((2-(3-cyclopropylureido)pyridin-4-yl)methyl)piperazin-1-yl)-N,6-dimelhylpicolinamide (Compound 46)

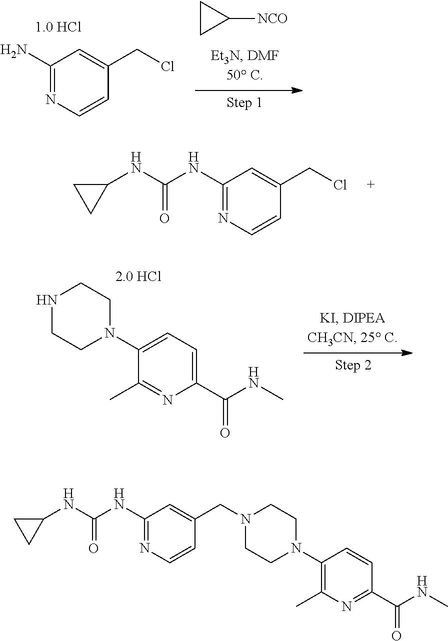

Step 1: Synthesis of 1-(4-(chloromethyl)pyridin-2-yl)-3-cyclopropylurea: To a stirred solution of 4-(chloromethyl)pyridin-2-amine hydrochloride (100 mg, 0.56 mmol) in DMF (1.5 mL) was added cyclopropyl isocyanate (55.7 mg, 0.67 mmol), Et₃N (113 mg, 1.12 mmol) and the reaction was stirred at 50° C. for 3 h. The reaction was quenched with aqueous NH₄Cl, extracted with EtOAc (20 mL×2). The combined organic layers were washed with brine, dried over Na₂SO₄, filtered and the filtrate was concentrated under reduced pressure. The residue was purified by silica gel chromatography to afford the title compound (130 mg, crude). LCMS (m/z): 226.1 [M+H]⁺.

Step 2: Synthesis of 5-(4-((2-(3-cyclopropylureido)pyridin-4-yl)methyl)piperazin-1-yl)-N,6-dimethylpicolinamide: A mixture of 1-(4-(chloromethyl)pyridin-2-yl)-3-cyclopropyl urea (130 mg, 0.58 mmol), N,6-dimethyl-5-(piperazin-1-yl)picolinamide dihydrochloride (178 mg, 0.58 mmol), KI (143 mg, 0.86 mmol) and DIPEA (744 mg, 5.76 mmol) in CH₃CN (5 mL) was stirred at 25° C. for 16 h. The reaction mixture was concentrated under reduced pressure and the residue was purified by silica gel chromatography to afford the crude product, which was purified by prep-HPLC to afford the title compound (44 mg, 19%). LCMS (m/z): 424.3 [M+H]; ¹HNMR (400 MHz, DMSO-d₆) δ 9.07 (s, 1H), 8.45-8.39 (m, 1H), 8.23-8.15 (m, 1H), 8.11 (d, J=5.2 Hz, 1H), 7.79 (d, J=8.3 Hz, 1H), 7.49 (d, J=8.3 Hz, 1H), 7.41 (s, 1H)), 6.91 (d, J=5.2 Hz, 1H), 3.52 (s, 2H), 2.99-2.92 (m, 4H), 2.80 (d, J=4.9 Hz, 3H), 2.63-2.53 (m, 5H), 2.49 (s, 3H), 0.68-0.63 (m, 2H), 0.46-0.40 (m, 2H).

### Example S-12: Synthesis of N,6-dimethyl-5-(4-((2-(3-methylureido)pyridin-4-yl)methyl)piperazin-1-yl)picolinamide trifluoroacetate (Compound 42)

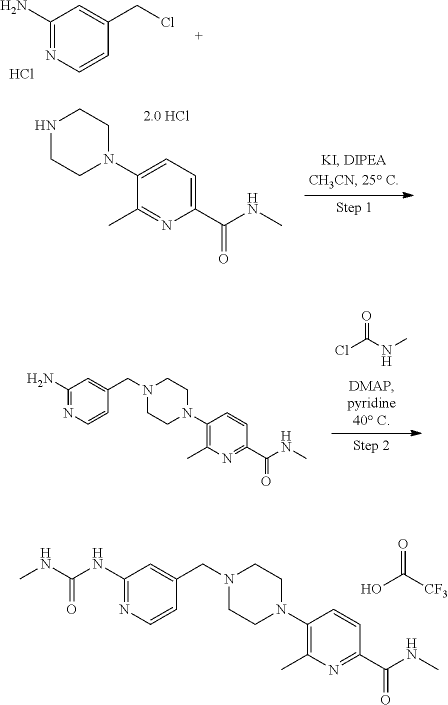

Step 1: Synthesis of 5-(4-((2-aminopyridin-4-yl)methyl)piperazin-1-yl)-N,6-dimethyl picolinamide: A mixture of 4-(chloromethyl)pyridin-2-amine hydrochloride (150 mg, 0.84 mmol), N,6-dimethyl-5-(piperazin-1-yl)picolinamide dihydrochloride (258 mg, 0.84 mmol), KI (209 mg, 1.26 mmol) and DIPEA (1.09 g, 8.4 mmol) in CH₃CN (15 mL) was stirred at 25° C. for 16 h. The reaction mixture was concentrated under reduced pressure and the residue was purified by silica gel chromatography to afford the title compound (130 mg, 45%). LCMS (m/z): 341.3 [M+H]⁺.

Step 2: Synthesis of N,6-dimethyl-5-(4-((2-(3-methylureido)pyridin-4-yl)methyl) piperazin-1-yl)picolinamide trifluoroacetate: To a mixture of 5-(4-((2-aminopyridin-4-yl)methyl)piperazin-1-yl)-N,6-dimethylpicolinamide (60 mg, 0.176 mmol) in pyridine (2 mL) was added DMAP (2 mg, 0.0176 mmol), N-methylcarbamoyl chloride (25 mg, 0.264 mmol) and the reaction was stirred at 40° C. for 48 h. The reaction mixture was concentrated under reduced pressure and the residue was purified by prep-HPLC to afford the title compound (5 mg, 5%). LCMS (m/z): 398.0 [M+H]⁺, ¹HNMR (400 MHz, CD₃OD) δ 8.35 (d, J=5.4 Hz, 1H), 7.93 (d, J=8.3 Hz, 1H), 7.59 (d, 1=8.3 Hz, 1H), 7.47 (s, 1H), 7.21 (d, J=5.4 Hz, 1H), 4.42 (s, 2H), 3.57-3.43 (m, 4H), 3.32-3.22 (m, 4H), 2.97 (s, 3H), 2.89 (s, 3H), 2.61 (s, 3H).

### Example S-13: Synthesis of 5-(4-((2-(3-ethylureido)pyridin-4-yl)methyl)piperazin-1-yl)-6-fluoro-N-methylpicolinamide (Compound 9)

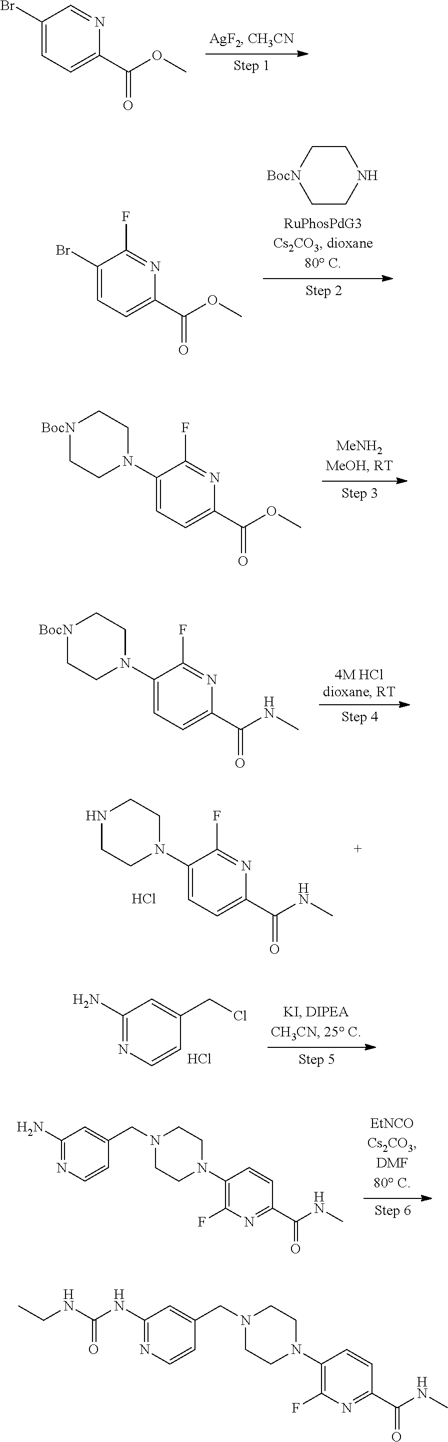

Step 1: Synthesis of methyl 5-bromo-6-fluoropicolinate: To a stirred solution of methyl 5-bromopicolinate (7 g, 32.4 mmol) in CH₃CN (50 mL) was added AgF₂ (23.6 g, 162 mmol) and the reaction was stirred at 25° C. for 18 h. The reaction mixture was filtrated, the filter cake was washed with MeOH (50 mL×3), and the filtrate was concentrated under reduced pressure. The crude residue was purified by silica gel chromatography to afford the title compound (2.9 g, 38%). LCMS (m/z): 234.0 [M+H]⁺.

Step 2: Synthesis of tert-butyl 4-(2-fluoro-6-(methoxycarbonyl)pyridin-3-yl)piperazine-1-carboxylate: To a stirred solution of methyl 5-bromo-6-fluoropicolinate (2.9 g, 12.39 mmol) in dioxane (50 mL) were added tert-butyl piperazine-1-carboxylate (2.77 g, 14.87 mmol), RuPhos Pd G3 (1.04 g, 1.24 mmol), Cs₂CO₃ (8.08 g, 24.8 mmol) and the reaction was stirred at 80° C. for 18 h under N2 atmosphere. The reaction mixture was quenched with aqueous NH₄Cl to pH 7 and extracted with EtOAc (50 mL×2). The combined organic layers were washed with brine, dried over Na₂SO₄, filtered and the filtrate was concentrated under reduced pressure. The residue was purified by silica gel chromatography to afford the title compound (2 g, 48%). LCMS (m/z): 340.2 [M+H]⁺.

Step 3: Synthesis of tert-butyl 4-(2-fluoro-6-(methylcarbamoyl)pyridin-3-yl) piperazine-1-carboxylate: To a stirred solution of tert-butyl 4-(2-fluoro-6-(methoxycarbonyl) pyridin-3-yl)piperazine-1-carboxylate (2 g, 5.89 mmol) in MeOH (10 mL) was added 33% methylamine in MeOH (10 mL) and the reaction was stirred at 25° C. for 18 h. The reaction mixture was concentrated under reduced pressure and the residue was purified by silica gel chromatography to afford the title compound (1.98 g, 99%). LCMS (m/z): 235.2 [M+H]⁺.

Step 4: Synthesis of 6-fluoro-N-methyl-5-(piperazin-1-yl)picolinamide hydrochloride: To a stirred solution of tert-butyl 4-(2-fluoro-6-(methylcarbamoyl)pyridin-3-yl) piperazine-1-carboxylate (1.98 g, 5.85 mmol) in DCM (15 mL) was added 4M HCl in dioxane (5 mL) and the reaction was stirred at 25° C. for 5 h. The reaction mixture was concentrated under reduced pressure to afford the title compound (1.5 g, 93%). LCMS (m/z): 239.2 [M+H]⁺.

Step 5: Synthesis of 5-(4-((2-aminopyridin-4-yl)methyl)piperazin-1-yl)-6-fluoro-N-methyl picolinamide: To a stirred solution of 6-fluoro-N-methyl-5-(piperazin-1-yl)picolinamide hydrochloride (200 mg, 0.73 mmol) in CH₃CN (4 mL) were added 4-(chloromethyl)pyridin-2-amine hydrochloride (131 mg, 0.73 mmol), DIPEA (282 mg, 2.18 mmol), KI (121 mg, 0.73 mmol) and the reaction was stirred at 25° C. for 18 h. The reaction mixture was concentrated under reduced pressure and the residue was purified by silica gel chromatography to afford the title compound (170 mg, 68%). LCMS (m/z): 345.2 [M+H]⁺.

Step 6: Synthesis of 5-(4-((2-(3-ethylureido)pyridin-4-yl)methyl)piperazin-1-yl)-6-fluoro-N-methylpicolinamide: To a stirred solution of 5-(4-((2-aminopyridin-4-yl)methyl) piperazin-1-yl)-6-fluoro-N-methylpicolinamide (160 mg, 0.46 mmol) in DMF (3 mL) were added ethyl isocyanate (50 mg, 0.70 mmol), Cs₂CO₃ (303 mg, 0.93 mmol), and the resulting reaction was stirred at 80° C. for 4 h. The reaction mixture was filtered, the filter cake was washed with MeOH (3 mL×2), and the filtrate was concentrated under reduced pressure. The residue was purified by silica gel chromatography to afford the crude product which was purified by Prep-HPLC to afford the title compound (54 mg, 28%). LCMS (m/z): 416.1 [M+H]⁺; ¹HNMR (400 MHz, DMSO-d₆) δ 9.13 (s, 1H), 8.40 (q, J=4.5 Hz, 1H), 8.22-8.13 (m, 1H), 8.11 (d, J=5.2 Hz, 1H), 7.85 (dd, J=8.0, 1.1 Hz, 1H), 7.57 (dd, J=10.6, 8.2 Hz, 1H), 7.34 (s, J H), 6.89 (dd, J=5.2, 1.0 Hz, 1H), 3.50 (s, 2H), 3.32 (s, 2H), 3.21-3.18 (m, 4H), 2.77 (d, J=4.8 Hz, 3H), 2.60-2.53 (m, 4H), 1.09 (t, J=7.2 Hz, 3H).

### Example S-14: Synthesis of 1-(4-((4-(5-cyanopyridin-2-yl) piperazin-1-yl) methyl) pyridin-2-yl)-3-ethylurea (Compound 263)

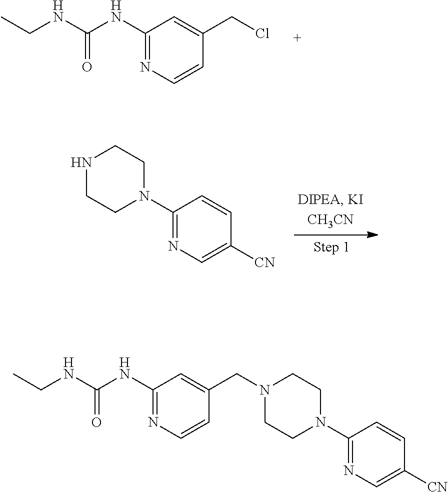 [SMILES: CCNC(=O)Nc1cc(CCl)ccn1 ; N#Cc1ccc(N2CCNCC2)nc1 ; CCNC(=O)Nc1cc(CN2CCN(c3ccc(C#N)cn3)CC2)ccn1]

Step 1: Synthesis of 1-(4-((4-(5-cyanopyridin-2-yl) piperazin-1-yl) methyl) pyridin-2-yl)-3-ethylurea: To a stirred solution of 1-(4-(chloromethyl) pyridin-2-yl)-3-ethylurea (80 mg, 0.37 mmol) in CH₃CN (2 mL) were added 6-(piperazin-1-yl)nicotinonitrile (70 mg, 0.37 mmol), DIPEA (145 mg, 1.12 mmol), KI (62 mg, 0.37 mmol) and the reaction mixture was stirred at 25° C. for 18 h. The reaction mixture was quenched with water and extracted with MeOH:DCM (1:10, 5 mL×2). The combined organic layers were washed with brine, dried over Na₂SO₄, filtered and the filtrate was concentrated under reduced pressure. The residue was purified by silica gel chromatography to afford the crude product which was purified by Prep-HPLC to afford the title compound (28 mg, 21%). LCMS (m/z): 366.1 [M+H]⁺; ¹HNMR (400 MHz, CD₃OD) δ 8.40 (d, J=2.1 Hz, 1H), 8.16 (d, J=5.3 Hz, 1H), 7.73 (dd, J=9.1, 2.3 Hz, 1H), 7.14 (s, 1H), 7.01 (d, J=5.2 Hz, 1H), 6.86 (d, J=9.1 Hz, 1H), 3.81-3.70 (m, 4H), 3.58 (s, 2H), 3.38-3.34 (m, 2H), 2.65-2.52 (m, 4H), 1.23 (t, J=7.2 Hz, 3H).

### Example S-15: Synthesis of N-methyl-5-(piperazin-1-yl)-6-(trifluoromethyl)picolinamide hydrochloride

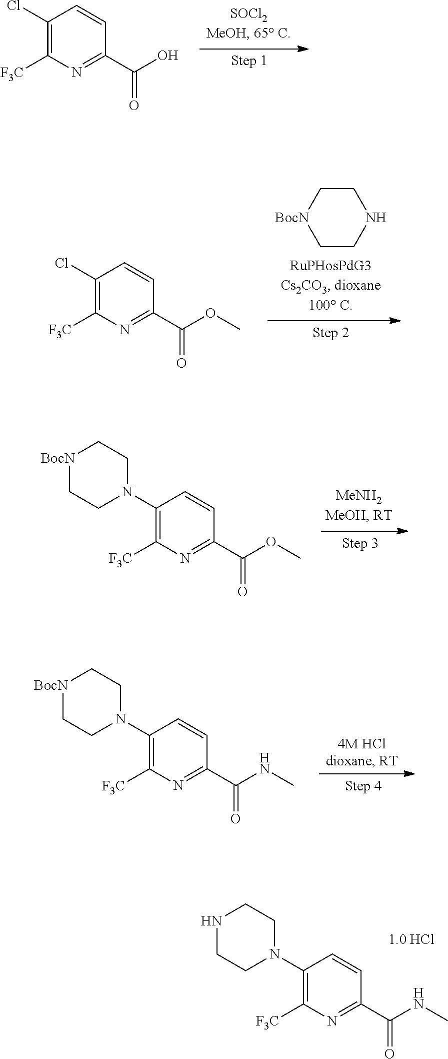 [SMILES: O=C(O)c1ccc(Cl)c(C(F)(F)F)n1 ; CC(C)(C)OC(=O)N1CCNCC1 ; COC(=O)c1ccc(Cl)c(C(F)(F)F)n1 ; COC(=O)c1ccc(N2CCN(C(=O)OC(C)(C)C)CC2)c(C(F)(F)F)n1 ; CNC(=O)c1ccc(N2CCN(C(=O)OC(C)(C)C)CC2)c(C(F)(F)F)n1 ; CNC(=O)c1ccc(N2CCNCC2)c(C(F)(F)F)n1]

Step 1: Synthesis of methyl 5-chloro-6-(trifluoromethyl)picolinate. To a solution of 5-chloro-6-(trifluoromethyl)picolinic acid (900 mg, 3.99 mmol) in MeOH (10 mL) was added SOCl₂ (0.6 mL, 7.98 mmol) and the reaction mixture was stirred at 65° C. for 2 h before it was concentrated under reduced pressure and purified by silica gel chromatography to afford the title compound (900 mg, 94%). LCMS (m/z): 239.8 [M+H]⁺.

Step 2: Synthesis of tert-butyl 4-(6-(methoxycarbonyl)-2-(trifluoromethyl)pyridin-3-yl) piperazine-1-carboxylate. To a solution of methyl 5-chloro-6-(trifluoromethyl)picolinate (900 mg, 3.76 mmol) in dioxane (10 mL) was added tert-butyl piperazine-1-carboxylate (840 mg, 4.51 mmol), Cs₂CO₃ (2.44 g, 7.49 mmol), RuPhosPdG3 (316 mg, 0.38 mmol) and the reaction mixture was stirred at 100° C. for 10 h under nitrogen atmosphere. The reaction mixture was concentrated under reduced pressure and purified by silica gel chromatography to afford the title compound (490 mg, 33%). LCMS (m/z): 390.1 [M+H]⁺.

Step 3: Synthesis of tert-butyl 4-(6-(methylcarbamoyl)-2-(trifluoromethyl)pyridin-3-yl)piperazine-1-carboxylate. To a solution of tert-butyl 4-(6-(methoxycarbonyl)-2-(trifluoromethyl)pyridin-3-yl) piperazine-1-carboxylate (40 mg, 0.10 mmol) in MeOH (1 mL) was added methylamine (33% in methanol, 2 mL) and the reaction mixture was stirred at 25° C. for 2 h before it was concentrated under reduced pressure to afford the title compound (39 mg, 98%). LCMS (m/z): 332.9 [M+H]⁺.

Step 4: Synthesis of N-methyl-5-(piperazin-1-yl)-6-(trifluoromethyl)picolinamide hydrochloride. A solution of tert-butyl 4-(6-(methylcarbamoyl)-2-(trifluoromethyl)pyridin-3-yl) piperazine-1-carboxylate (39 mg, 0.1 mmol) in 4M HCl in dioxane (3 mL) was stirred at 25° C. for 2 h. The reaction was concentrated to give the title compound (50 mg, crude). LCMS (m/z): 289.2 [M+H]⁺.

### Example S-16: Synthesis of 5-(4-((2-(3-ethylureido)pyridin-4-yl)methyl)piperazin-1-yl)-N-methyl-6-(trifluoromethyl)picolinamide trifluoroacetate (Compound 21)

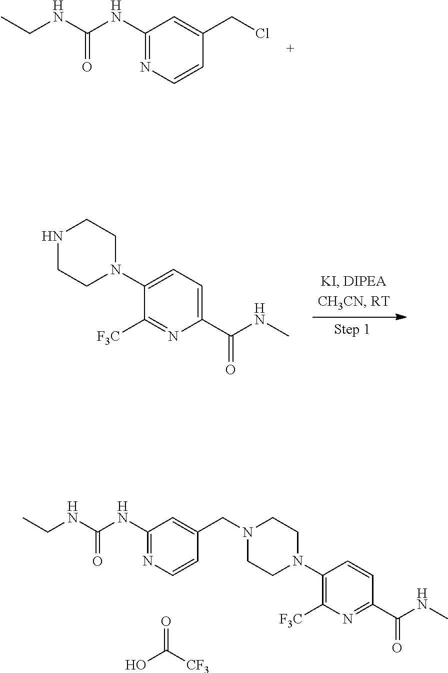 [SMILES: CCNC(=O)Nc1cc(CCl)ccn1 ; CNC(=O)c1ccc(N2CCNCC2)c(C(F)(F)F)n1 ; CCNC(=O)Nc1cc(CN2CCN(c3ccc(C(=O)NC)nc3C(F)(F)F)CC2)ccn1 ; O=C(O)C(F)(F)F]

Step 1: Synthesis of 5-(4-((2-(3-ethylureido)pyridin-4-yl)methyl)piperazin-1-yl)-N-methyl-6-(trifluoromethyl)picolinamide trifluoroacetate: A mixture of 1-(4-(chloromethyl)pyridin-2-yl)-3-ethylurea (64 mg, 0.30 mmol), N-methyl-5-(piperazin-1-yl)-6-(trifluoromethyl)picolinamide (86 mg, 0.30 mmol), KI (100 mg, 0.6 mmol) and DIPEA (76 mg, 0.6 mmol) in CH₃CN (3 mL) was stirred at RT for 16 h. The reaction mixture was concentrated under reduced pressure and the residue was purified by silica gel chromatography to afford the crude product, which was purified by Prep-HPLC to afford the title compound (21 mg, 12%). LCMS (m/z): 466.3 [M+H]⁺; ¹HNMR (400 MHz, DMSO-d₆) δ 10.14 (br s, 1H), 9.43 (br s, 1H), 8.50 (d, J=4.8 Hz, 1H), 8.27 (dd, J=14.2, 6.8 Hz, 2H), 8.10 (d, J=8.5 Hz, 1H), 7.71 (s, 1H), 7.62 (s, 1H), 7.11 (s, 1H), 4.42 (s, 2H), 3.45 (s, 2H), 3.06-3.37 (m, 8H), 2.83 (d, J=4.8 Hz, 3H), 1.09 (t, J=7.2 Hz, 3H).

### Example S-17: Synthesis of (R)-N-methyl-1,2,3,4,4a,5-hexahydropyrazino[1,2-d]pyrido[2,3-b]oxazine-8-carboxamide

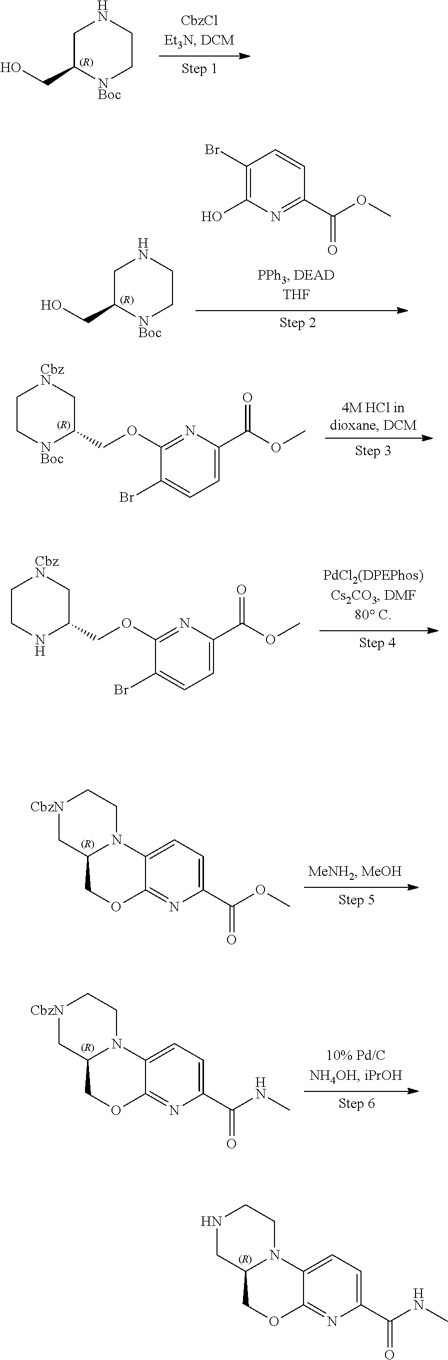

Step 1: Synthesis of 4-benzyl 1-(tert-butyl) (R)-2-(hydroxymethyl)piperazine-1,4-dicarboxylate. To a stirred solution of tert-butyl (R)-2-(hydroxymethyl)piperazine-1-carboxylate (4 g, 18.49 mmol) in DCM (100 mL) was added Et₃N (7.7 mL, 55.54 mmol), CbzCl (5.3 mL, 36.99 mmol) and the reaction mixture was stirred at 25° C. for 18 h before it was quenched with water (100 mL), extracted with CH₂Cl₂ (50 mL×2). The combined organic layers were washed with brine, dried over Na₂SO₄, filtered and the filtrate was concentrated under reduced pressure. The residue was purified by silica gel chromatography to afford the title compound (3.8 g, 590). LCMS (m/z): 373.3 [M+Na]⁺.

Step 2: Synthesis of 4-benzyl 1-(tert-butyl) (R)-2-(((3-bromo-6-(methoxycarbonyl) pyridin-2-yl)oxy)methyl)piperazine-1,4-dicarboxylate. To a stirred solution of DEAD (8.5 mL, 53.98 mmol) in THF (50 mL) at 0° C. was added PPh₃ (17 g, 64.81 mmol) and the reaction mixture was stirred at the same temperature for 1 h. Then a solution of 4-benzyl 1-(tert-butyl) (R)-2-(hydroxymethyl)piperazine-1,4-dicarboxylate (3.8 g, 10.84 mmol) and methyl 5-bromo-6-hydroxypicolinate (2.51 g, 10.82 mmol) in THF (50 mL) was added at 0° C. and the resulting solution was stirred at 25° C. for 2 h. The reaction mixture was concentrated under reduced pressure and purified by silica gel chromatography to furnish the title compound (4.6 g, 75%). LCMS (m/z): 586.2 [M+Na]⁺.

Step 3: Synthesis of benzyl (R)-3-(((3-bromo-6-(methoxycarbonyl)pyridin-2-yl)oxy) methyl)piperazine-1-carboxylate. To a stirred solution of 4-benzyl 1-(tert-butyl) (R)-2-(((3-bromo-6-(methoxycarbonyl)pyridin-2-yl)oxy)methyl)piperazine-1,4-dicarboxylate (4.6 g, 8.15 mmol) in DCM (50 mL) was added 4N HCl in dioxane (50 mL) and the reaction mixture was stirred at 25° C. for 18 h. Then it was neutralized to pH=8 with aqueous Na₂CO₃, extracted with CH₂C₁₋₂ (50 mL×2). The combined organic layers were washed with brine, dried over Na₂SO₄, filtered and the filtrate was concentrated under reduced pressure and purified by silica gel chromatography to afford the title compound (3.6 g, 95%). LCMS (m/z): 464.2 [M+H]⁺.

Step 4: Synthesis of 3-benzyl 8-methyl (R)-1,2,4a,5-tetrahydropyrazino[1,2-d]pyrido [2,3-b][1,4]oxazine-3,8(4H)-dicarboxylate. To a stirred solution of benzyl (R)-3-(((3-bromo-6-(methoxycarbonyl)pyridin-2-yl)oxy)methyl)piperazine-1-carboxylate (2 g, 4.31 mmol) in DMF (50 mL) was added PdCl₂DPEPhos (0.92 g, 1.29 mmol), Cs₂CO₃ (4.2 g, 12.89 mmol) and the reaction mixture was stirred at 80° C. for 18 h under N2. The reaction mixture was quenched with aqueous NH₄Cl, extracted with EtOAc (50 mL×2). The combined organic layers were washed with brine, dried over Na₂SO₄, filtered and the filtrate was concentrated under reduced pressure and purified by silica gel chromatography to produce the title compound (600 mg, 36%). LCMS (m/z): 384.2 [M+H]⁺.

Step 5: Synthesis of benzyl (R)-8-(methylcarbamoyl)-1,2,4a,5-tetrahydropyrazino [1,2-d]pyrido[2,3-b][1,4]oxazine-3(4H)-carboxylate. To a stirred solution of 3-benzyl 8-methyl (R)-1,2,4a,5-tetrahydropyrazino[1,2-d]pyrido[2,3-b][1,4]oxazine-3,8(4H)-dicarboxylate (600 mg, 1.56 mmol) in MeOH (3 mL) was added MeNH₂ (33% in MeOH, 3 mL) and the reaction mixture was stirred at 25° C. for 18 h. The reaction mixture was concentrated under reduced pressure and purified by silica gel chromatography to afford the title compound (420 mg, 70%). LCMS (m/z): 383.3 [M+H]⁺.

Step 6: Synthesis of (R)-N-methyl-1,2,3,4,4a,5-hexahydropyrazino[1,2-d]pyrido[2,3-b][1,4]oxazine-8-carboxamide. To a stirred solution of benzyl (R)-8-(methylcarbamoyl)-1,2,4a,5-tetrahydropyrazino[1,2-d]pyrido[2,3-b][1,4]oxazine-3(4H)-carboxylate (420 mg, 1.1 mmol) in i-PrOH (5 mL) was added NH₄OH (1 mL), 10% Pd/C (117 mg) and the reaction mixture was stirred at RT for 4 h under H₂. The reaction mixture was filtered, and filter cake was washed with MeOH (5 mL×3). The filtrate was concentrated under reduced pressure and purified by silica gel chromatography to afford the title compound (260 mg, 95%). LCMS (m/z): 249.2 [M+H]⁺.

### Example S-18: Synthesis of (R)-3-((2-(3-ethylureido)pyridin-4-yl)methyl)-N-methyl-1,2,3,4,4a,5-hexahydropyrazino[1,2-d]pyrido[2,3-b][1,4]oxazine-8-carboxamide (Compound 264)

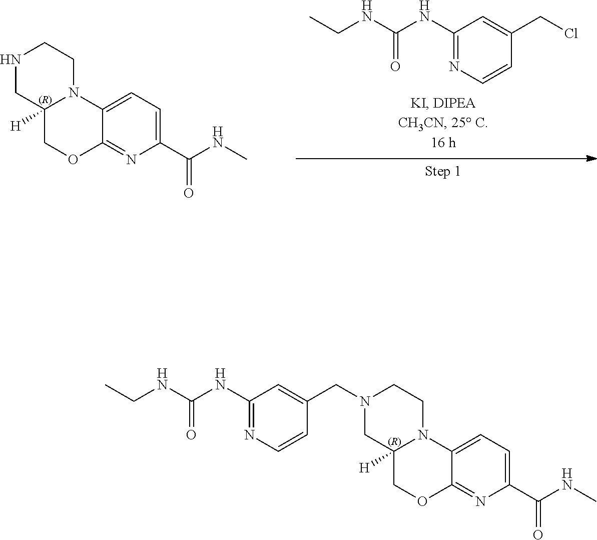

Step 1: Synthesis of (R)-3-((2-(3-ethylureido)pyridin-4-yl)methyl)-N-methyl-1,2,3,4,4a,5-hexahydropyrazino[1,2-d]pyrido[2,3-b][1,4]oxazine-8-carboxamide: A mixture of 1-(4-(chloromethyl)pyridin-2-yl)-3-ethylurea (35 mg, 0.16 mmol), (R)-N-methyl-1,2,3,4,4a,5-hexahydropyrazino[1,2-d]pyrido[2,3-b][1,4]oxazine-8-carboxamide (46 mg, 0.18 mmol), KI (42 mg, 0.25 mmol) and DIPEA (106 mg, 0.82 mmol) in CH₃CN (3 mL) was stirred at 25° C. for 16 h. The reaction mixture was concentrated under reduced pressure and the residue was purified by silica gel chromatography to afford the crude product, which was purified by Prep-HPLC to afford the title compound (22 mg, 32%). LCMS (m/z): 426.2 [M+H]⁺; ¹HNMR (400 MHz, DMSO-d₆) δ 9.10 (s, 1H), 8.19-8.10 (m, 2H), 8.08 (d, J=5.2 Hz, 1H), 7.49 (d, J=8.1 Hz, 1H), 7.30-7.23 (m, 2H), 6.87-6.84 (m, 1H), 4.37 (dd, J=11.0, 2.8 Hz, 1H), 4.01 (dd, J=10.9, 9.1 Hz, 1H), 3.75 (d, J=13.0 Hz, 1H), 3.47 (dd, J=33.1, 14.3 Hz, 2H), 3.22-3.10 (m, 3H), 2.93-2.73 (m, 3H), 2.70 (d, J=4.8 Hz, 3H), 2.21-2.13 (m, 1H), 1.77 (t, J=10.8 Hz, 1H), 1.04 (t, J=7.2 Hz, 3H).

### Example S-19: Synthesis of 5-(4-((2-((N-ethylsulfamoyl)amino)pyridin-4-yl)methyl)piperazin-1-yl)-N,6-dimethylpicolinamide (Compound 105)

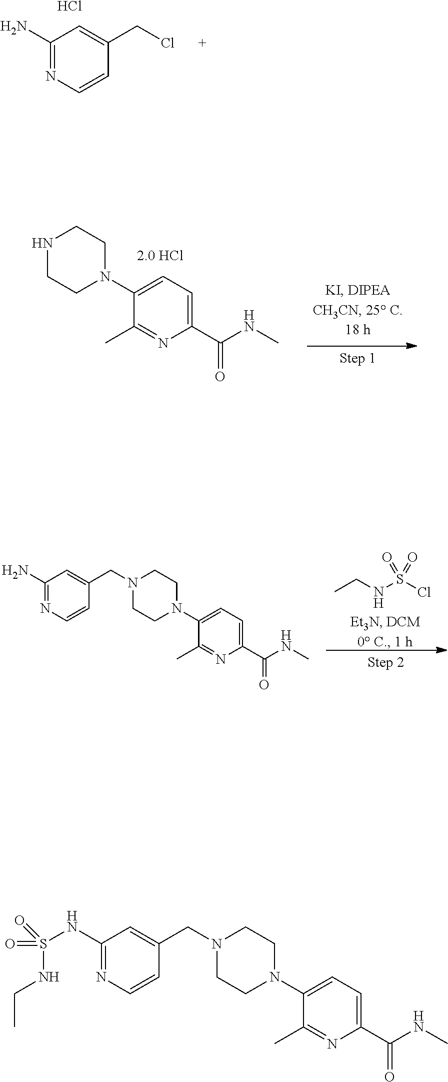

Step 1: Synthesis of 5-(4-((2-aminopyridin-4-yl) methyl) piperazin-1-yl)-N,6-dimethylpicolinamide: To a stirred solution of N,6-dimethyl-5-(piperazin-1-yl)picolinamide dihydrochloride (20 mg, 0.065 mmol) in CH₃CN (2 mL) was added 4-(chloromethyl)pyridin-2-amine hydrochloride (12 mg, 0.067 mmol), DIPEA (25 mg, 0.19 mmol), KI (11 mg, 0.066 mmol) and the reaction mixture was stirred at 25° C. for 18 h. The reaction mixture was quenched with aqueous NaHCO₃ to pH 8 and extracted with DCM (5 mL×2). The combined organic layers were washed with brine, dried over Na₂SO₄, filtered and the filtrate was concentrated under reduced pressure. The residue was purified by silica gel chromatography to afford the title compound (22 mg, 99%). LCMS (m/z): 341.1 [M+H]⁺.

Step 2: Synthesis of 5-(4-((2-((N-ethylsulfamoyl)amino)pyridin-4-yl)methyl) piperazin-1-yl)-N,6-dimethylpicolinamide: To a stirred solution of 5-(4-((2-aminopyridin-4-yl) methyl) piperazin-1-yl)-N,6-dimethylpicolinamide (22 mg, 0.065 mmol) in DCM (1 mL) were added ethylsulfamoyl chloride (10 mg, 0.071 mmol), Et₃N (13 mg, 0.13 mmol) and the reaction mixture was stirred at 0° C. for 1 h. The reaction mixture was concentrated under reduced pressure and the residue was purified by Prep-HPLC to afford the title compound (2 mg, 9%). LCMS (m/z): 448.0 [M+H]⁺;

¹HNMR (400 MHz, DMSO-d₆) δ 8.43 (q, J=4.7 Hz, 1H), 8.36 (s, 1H), 8.15 (d, J=5.1 Hz, 1H), 7.80 (d, J=8.2 Hz, 1H), 7.49 (d, J=8.3 Hz, 1H), 7.22 (s, 1H), 7.03 (s, 1H), 6.95 (d, J=4.8 Hz, 1H), 3.54 (s, 2H), 3.01-2.93 (m, 4H), 2.90-2.86 (m, 2H), 2.80 (d, J=4.9 Hz, 3H), 2.64-2.54 (m, 4H), 2.49 (s, 3H), 0.98 (t, J=7.2 Hz, 3H).

### Example S-20: Synthesis of N-cyano-5-(4-((2-(3-ethylureido)pyridin-4-yl)methyl)piperazin-1-yl)-6-methylpicolinamide dihydrochloride (Compound 3)

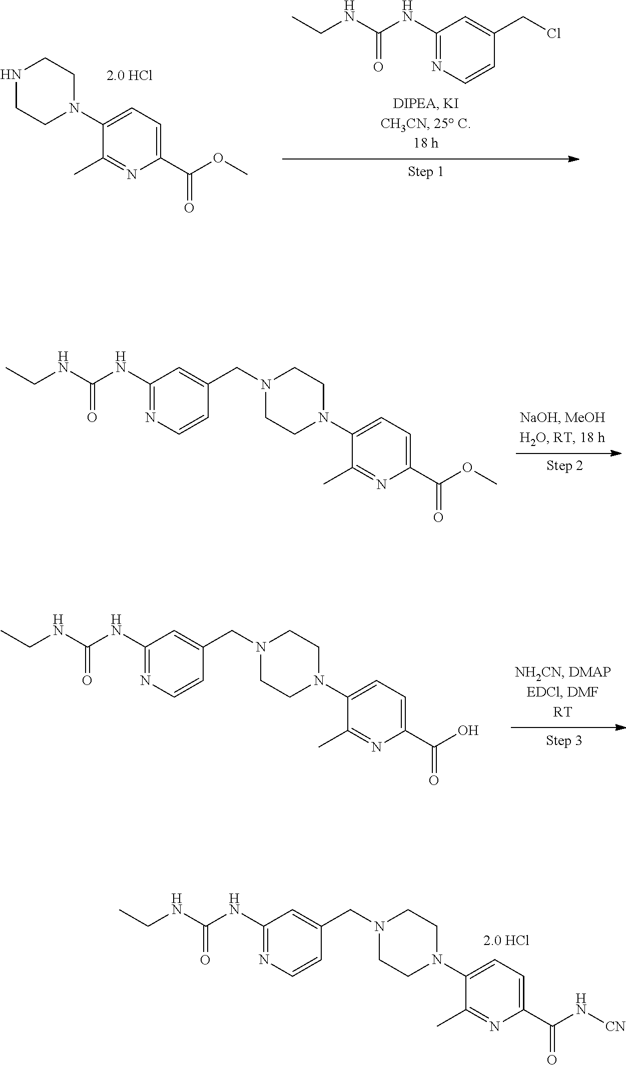

Step 1: Synthesis of methyl 5-(4-((2-(3-ethylureido)pyridin-4-yl)methyl)piperazin-1-yl)-6-methylpicolinate: To a stirred solution of methyl 6-methyl-5-(piperazin-1-yl) picolinate dihydrochloride (300 mg, 0.97 mmol) in CH₃CN (5 mL) was added 1-(4-(chloromethyl) pyridin-2-yl)-3-ethylurea (207 mg, 0.97 mmol), DIPEA (629 mg, 4.87 mmol), KI (161 mg, 0.97 mmol) and the reaction was stirred at 25° C. for 18 h. The reaction mixture was quenched with water and extracted with DCM (10 mL×2). The combined organic layers were washed with brine, dried over Na₂SO₄, filtered and the filtrate was concentrated under reduced pressure. The residue was purified by silica gel chromatography to afford the title compound (400 mg, 99.6%). LCMS (m/z): 413.2 [M+H]⁺.

Step 2: Synthesis of 5-(4-((2-(3-ethylureido)pyridin-4-yl)methyl)piperazin-1-yl)-6-methylpicolinic acid: To a stirred solution of methyl 5-(4-((2-(3-ethylureido)pyridin-4-yl)methyl)piperazin-1-yl)-6-methylpicolinate (380 mg, 0.92 mmol) in MeOH (4 mL) was added H₂O (1 mL) and NaOH (114 mg, 2.85 mmol) and the reaction was stirred at RT for 18 h. The reaction mixture was quenched with 1M HCl to pH 6. The reaction mixture was filtered, and the filter cake was washed with MeOH (5 mL×3). The filtrate was concentrated under reduced pressure and the residue was purified by C₁₈ chromatography with water/CH₃CN to afford the title compound (290 mg, 79%). LCMS (m/z): 399.1 [M+H]⁺

Step 3: Synthesis of N-cyano-5-(4-((2-(3-ethylureido)pyridin-4-yl)methyl)piperazin-1-yl)-6-methylpicolinamide dihydrochloride: To a stirred solution of 5-(4-((2-(3-ethylureido)pyridin-4-yl)methyl)piperazin-1-yl)-6-methylpicolinic acid (270 mg, 0.68 mmol) in DMF (10 mL) were added DMAP (182 mg, 1.49 mmol), EDCI (312 mg, 1.63 mmol), NH₂CN (31 mg, 0.75 mmol) and the resulting reaction was stirred at RT for 72 h. The reaction mixture was concentrated under reduced pressure and the residue was purified by Prep-HPLC to afford the title compound (11 mg, 3%). LCMS (m/z): 423.3 [M+H]⁺, ¹HNMR (400 MHz, DMSO-d₆) δ 9.15 (s, 1H), 8.21 (s, 1H), 8.11 (d, J=5.2 Hz, 1H), 7.80 (d, J=8.2 Hz, 1H), 7.40 (d, J=7.9 Hz, 1H), 7.33 (s, 1H), 7.28-6.95 (m, 2H), 6.90 (d, J=5.1 Hz, 1H), 3.52 (s, 2H), 3.25-3.14 (m, 2H), 2.99-2.87 (m, 4H), 2.64-2.54 (m, 4H), 2.45 (s, 3H), 1.09 (t, J=7.2 Hz, 3H).

### Example S-21: Synthesis of 5-(4-((2-(3-ethylureido)-3-fluoropyridin-4 yl)methyl)piperazin-1-yl)-N,6-dimethylpicolinamide (Compound 82)

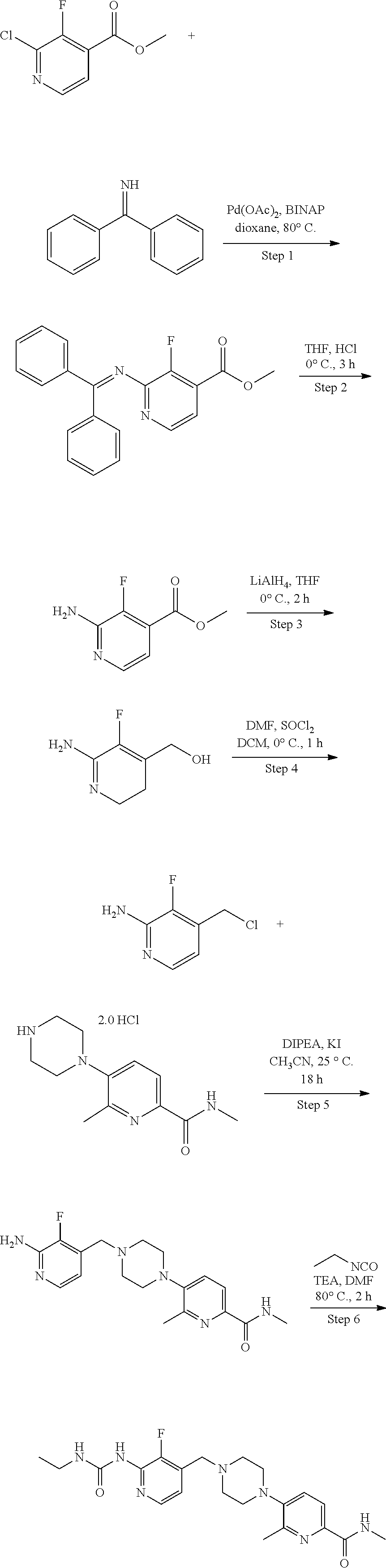

Step 1: Synthesis of methyl 2-((diphenylmethylene)amino)-3-fluoroisonicotinate: To a stirred solution of methyl 2-chloro-3-fluoroisonicotinate (4 g, 21.1 mmol) in dioxane (40 mL) were added diphenylmethanimine (5.7 g, 31.7 mmol), Pd(OAc)₂ (951 mg, 4.2 mmol), BINAP (5.2 g, 8.4 mmol), Cs₂CO₃ (13.8 g, 42.4 mmol) and the reaction mixture was stirred at 80° C. for 18 h under N2 atmosphere. The reaction mixture was quenched with aqueous NH₄Cl and extracted with EtOAc (50 mL×2). The combined organic layers were washed with brine, dried over Na₂SO₄, filtered and the filtrate was concentrated under reduced pressure. The residue was purified by silica gel chromatography to afford the title compound (2 g, 28%). LCMS (m/z): 335.0 [M+H]⁺.

Step 2: Synthesis of methyl 2-amino-3-fluoroisonicotinate: To a stirred solution of methyl 2-((diphenylmethylene)amino)-3-fluoroisonicotinate (2 g, 5.98 mmol) in THF (16 mL) was added Conc HCl (4 mL) and the reaction was stirred at 0° C. for 3 h. The reaction mixture was quenched with aqueous NaHCO₃ to pH 8 and extracted with MeOH:DCM (1:10, 50 mL×2). The combined organic layers were washed with brine, dried over Na₂SO₄, filtered and the filtrate was concentrated under reduced pressure. The residue was purified by silica gel chromatography to afford the title compound (600 mg, 59%). LCMS (m/z): 171.2 [M+H]⁺.

Step 3: Synthesis of (2-amino-3-fluoropyridin-4-yl) methanol: To a stirred solution of methyl 2-amino-3-fluoroisonicotinate (600 mg, 3.53 mmol) in THF (10 mL) was added LiAlH₄ (3.6 mL, 1 M in THF) at 0° C. and the reaction was stirred at 0° C. for 2 h. The reaction mixture was quenched with Na₂SO₄·10H₂O (1 g). The reaction mixture was filtered, and the filter cake was washed with MeOH:DCM (1:10, 10 mL×3). The filtrate was concentrated under reduced pressure and the crude residue was purified by silica gel chromatography to afford the title compound (210 mg, 42%). LCMS (m/z): 143.1 [M+H]⁺.

Step 4: Synthesis of 4-(chloromethyl)-3-fluoropyridin-2-amine: To a stirred solution of (2-amino-3-fluoropyridin-4-yl) methanol (310 mg, 2.18 mmol) in DCM (5 mL) at 0° C. were added DMF (32 mg, 0.44 mmol) and SOCl₂ (259 mg, 2.18 mmol) and the resulting reaction mixture was stirred at 0° C. for 1 h. The reaction mixture was quenched with aqueous NaHCO₃ to pH 8 and extracted with MeOH:DCM (1:10, 10 mL×3). The combined organic layers were washed with brine, dried over Na₂SO₄, filtered and the filtrate was concentrated under reduced pressure. The residue was purified by silica gel chromatography to afford the title compound (180 mg, 51%). LCMS (m/z): 161.1 [M+H]⁺.

Step 5: Synthesis of 5-(4-((2-amino-3-fluoropyridin-4-yl)methyl)piperazin-1-yl)-N,6-dimethylpicolinamide: To a stirred solution of 4-(chloromethyl)-3-fluoropyridin-2-amine (180 mg, 1.12 mmol) in CH₃CN (5 mL) were added N,6-dimethyl-5-(piperazin-1-yl)picolinamide dihydrochloride (344 mg, 1.12 mmol), DIPEA (725 mg, 5.61 mmol), KI (186 mg, 1.12 mmol) and the reaction mixture was stirred at 25° C. for 18 h. The reaction mixture was concentrated under reduced pressure and the crude residue was purified by silica gel chromatography to afford the title compound (100 mg, 25%). LCMS (m/z): 359.3 [M+H]⁺.

Step 6: Synthesis of 5-(4-((2-(3-ethylureido)-3-fluoropyridin-4-yl)methyl)piperazin-1-yl)-N,6-dimethylpicolinamide: To a stirred solution of 5-(4-((2-amino-3-fluoropyridin-4-yl)methyl)piperazin-1-yl)-N,6-dimethylpicolinamide (80 mg, 0.22 mmol) in DMF (1 mL) were added ethyl isocyanate (18 mg, 0.25 mmol), Cs₂CO₃ (147 mg, 0.45 mmol) and the resulting reaction mixture was stirred at 80° C. for 2 h. The reaction mixture was quenched with aqueous NH₄Cl to pH 7 and extracted with MeOH:DCM (1:10, 5 mL×4). The combined organic layers were washed with brine, dried over Na₂SO₄, filtered and the filtrate was concentrated under reduced pressure. The residue was purified by Prep-HPLC to afford the title compound (5 mg, 6%). LCMS (m/z): 430.1 [M+H]⁺; ¹HNMR (400 MHz, DMSO-d₆) δ 9.07 (s, 1H), 9.02-8.90 (m, 1H), 8.43 (q, J=4.6 Hz, 1H), 8.02 (d, J=5.1 Hz, 1H), 7.79 (d, J=8.3 Hz, 1H), 7.48 (d, J=8.3 Hz, 1H), 7.08 (t, J=4.9 Hz, 1H), 3.65 (s, 2H), 3.28-3.20 (m, 2H), 3.08-2.86 (m, 4H), 2.80 (d, J=4.9 Hz, 3H), 2.69-2.53 (m, 4H), 2.48 (s, 3H), 1.11 (t, J=7.2 Hz, 3H).

### Example S-22: Synthesis of 5-(4-((2-(3-ethylureido)-3-methylpyridin-4-yl) methyl) piperazin-1-yl)-N,6-dimethylpicolinamide hemiformate (Compound 260)

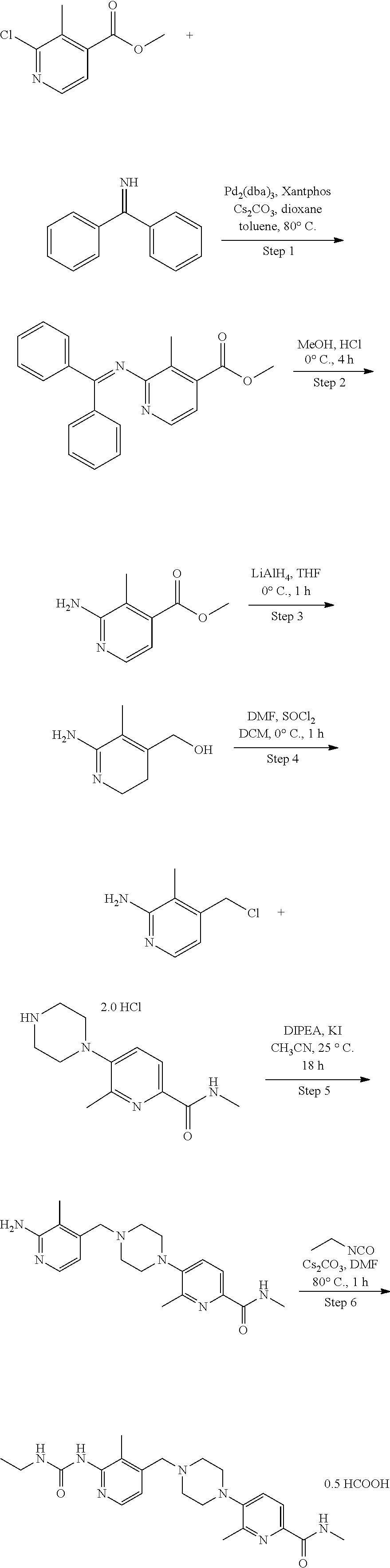

Step 1: Synthesis of methyl 2-((diphenyl methylene)amino)-3-methylisonicotinate: To a stirred solution of methyl 2-chloro-3-methylisonicotinate (300 mg, 1.62 mmol) in dioxane (5 mL) were added toluene (5 mL), diphenylmethanimine (323 mg, 1.78 mmol), Pd(dba)₂ (91 mg, 0.16 mmol), Xantphos (185 mg, 0.32 mmol), Cs₂CO₃ (1.06 g, 3.24 mmol) and the reaction mixture was stirred at 100° C. for 18 h under N2 atmosphere. The reaction mixture was quenched with water and extracted with EtOAc (20 mL×2). The combined organic layers were washed with brine, dried over Na₂SO₄, filtered and the filtrate was concentrated under reduced pressure. The residue was purified by silica gel chromatography to afford the title compound (280 mg, 52%). LCMS (m/z): 331.2 [M+H]⁺.

Step 2: Synthesis of methyl 2-amino-3-methylisonicotinate: To a stirred solution of methyl 2-((diphenyl methylene)amino)-3-methylisonicotinate (280 mg, 0.85 mmol) in MeOH (4 mL) was added Conc. HCl (1 mL) and the reaction mixture was stirred at 25° C. for 4 h. The reaction mixture was quenched with aqueous NaHCO₃ to pH 8 and extracted with DCM (20 mL×3). The combined organic layers were washed with brine, dried over Na₂SO₄, filtered and the filtrate was concentrated under reduced pressure. The residue was purified by silica gel chromatography to afford the title compound (140 mg, 99%). LCMS (m/z): 167.2 [M+H]⁺.

Step 3: Synthesis of (2-amino-3-methylpyridin-4-yl) methanol: To a stirred solution of methyl 2-amino-3-methylisonicotinate (140 mg, 0.84 mmol) in THF (4 mL) at 0° C. was added 1.0 M LiAlH₄ in THF (0.84 mL) and the reaction mixture was stirred at 0° C. for 1 h. The reaction mixture was quenched with Na₂SO₄·10H₂O (300 mg), filtered and the filtrate was concentrated under reduced pressure. The residue was purified by silica gel chromatography to afford the title compound (100 mg, 86%). LCMS (m/z): 139.2 [M+H]⁺.

Step 4: Synthesis of 4-(chloromethyl)-3-methylpyridin-2-amine: To a stirred solution of (2-amino-3-methylpyridin-4-yl) methanol (80 mg, 0.58 mmol) in DCM (2 mL) at 0° C. were added DMF (4 mg, 0.058 mmol) and SOCl₂ (69 mg, 0.58 mmol) and the reaction was stirred at the same temperature for 1 h. The reaction mixture was quenched with aqueous NaHCO₃ and extracted with DCM (5 mL/2). The combined organic layers were washed with brine, dried over Na₂SO₄, filtered and the filtrate was concentrated under reduced pressure. The crude residue was purified by silica gel chromatography to afford the title compound (60 mg, 66%). LCMS (m/z): 157.1 [M+H]⁺.

Step 5: Synthesis of 5-(4-((2-amino-3-methylpyridin-4-yl) methyl) piperazin-1-yl)-N,6-dimethylpicolinamide: To a stirred solution of 4-(chloromethyl)-3-methylpyridin-2-amine (60 mg, 0.38 mmol) in CH₃CN (1 mL) were added N,6-dimethyl-5-(piperazin-1-yl)picolinamide dihydrochloride (117 mg, 0.38 mmol), DIPEA (248 mg, 1.92 mmol), KI (63 mg, 0.38 mmol) and the reaction mixture was stirred at 25° C. for 18 h. The reaction mixture was concentrated under reduced pressure and the residue was purified by silica gel chromatography to afford the title compound (60 mg, 45%). LCMS (m/z): 355.1 [M+H]⁺.

Step 6: Synthesis of 5-(4-((2-(3-ethylureido)-3-methylpyridin-4-yl) methyl) piperazin-1-yl)-N,6-dimethylpicolinamide hemiformate: To a stirred solution of 5-(4-((2-amino-3-methylpyridin-4-yl)methyl)piperazin-1-yl)-N,6-dimethylpicolinamide (60 mg, 0.17 mmol) in DMF (1 mL) were added ethyl isocyanate (12 mg, 0.17 mmol), Cs₂CO₃ (111 mg, 0.34 mmol) and the reaction was stirred at 80° C. for 1 h. The reaction mixture was quenched with water and extracted with MeOH:DCM (1:10, 5 mL×2). The combined organic layers were washed with brine, dried over Na₂SO₄, filtered and the filtrate was concentrated under reduced pressure. The residue was purified by Prep-HPLC to afford the title compound (8 mg, 11%). LCMS (m/z): 426.3 [M+H]⁺; ¹HNMR (400 MHz, DMSO-d₆) δ 9.25 (t, J=4.9 Hz, 1H), 8.39 (q, J=4.6 Hz, 1H), 8.05 (s, 1H), 8.01 (d, J=5.1 Hz, 1H), 7.75 (d, J=8.2 Hz, 1H), 7.44 (d, J=8.3 Hz, 1H), 6.96 (d, J=5.2 Hz, 1H), 3.50 (s, 2H), 3.22-3.17 (m, 2H), 2.93-2.86 (m, 4H), 2.76 (d, J=4.9 Hz, 3H), 2.57-2.51 (m, 4H), 2.45 (s, 3H), 2.17 (s, 3H), 1.07 (t, J=7.2 Hz, 3H).

### Example S-23: Synthesis of 5-(4-((2-(3-ethylureido)-5-methylpyridin-4-yl) methyl) piperazin-1-yl)-N,6-dimethylpicolinamide formate (Compound 70)

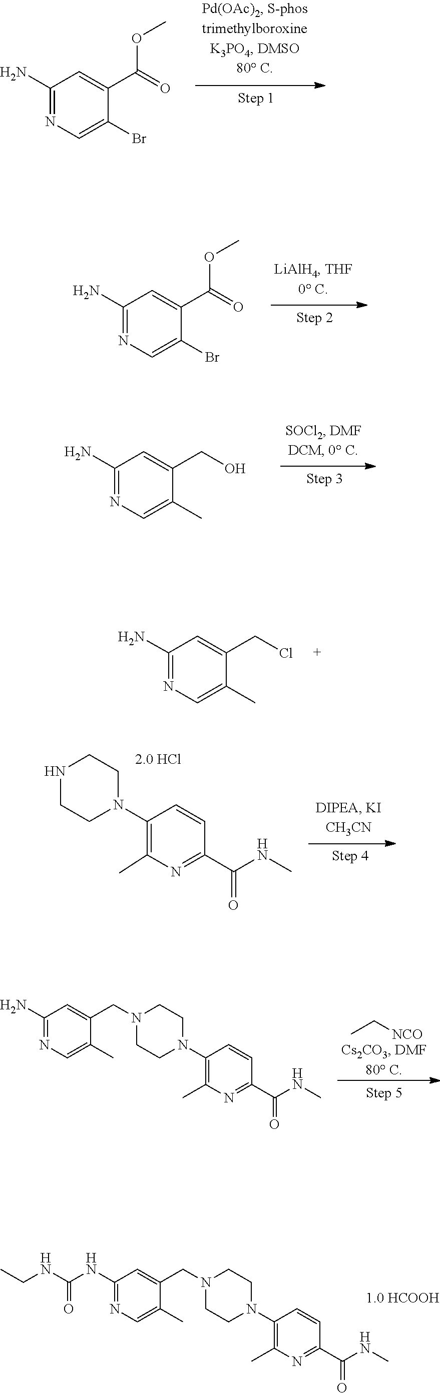

Step 1: Synthesis of methyl 2-amino-5-methylisonicotinate: To a stirred solution of methyl 2-amino-5-bromoisonicotinate (400 mg, 1.73 mmol) in DMSO (8 mL) were added trimethylboroxine (436 mg, 3.47 mmol), Pd(OAc)₂ (77 mg, 0.34 mmol), S-phos (283 mg, 0.69 mmol), K₃PO₄ (1.1 g, 5.19 mmol) and the reaction mixture was stirred at 80° C. for 18 h under N2 atmosphere. The reaction mixture was quenched with water and extracted with EtOAc (15 mL×3). The combined organic layers were washed with brine, dried over Na₂SO₄, filtered and the filtrate was concentrated under reduced pressure. The residue was purified by silica gel chromatography to afford the title compound (110 mg, 38%). LCMS (m/z): 167.2 [M+H]⁺.

Step 2: Synthesis of (2-amino-5-methylpyridin-4-yl) methanol: To a stirred solution of methyl 2-amino-5-methylpyridine-4-carboxylate (110 mg, 0.66 mmol) in THF (2 mL) at 0° C. was added 1.0 M LiAlH₄ in THF (0.7 mL) and the reaction mixture was stirred at the same temperature for 1 h. The reaction mixture was quenched with Na₂SO₄·10H₂O (500 mg), filtered and the filter cake was washed with MeOH:DCM (1:10, 5 mL×3). The filtrate was concentrated under reduced pressure and the residue was purified by silica gel chromatography to afford the title compound (80 mg, 88%). LCMS (m/z): 139.2 [M+H]⁺.

Step 3: Synthesis of 4-(chloromethyl)-5-methylpyridin-2-amine: To a stirred solution of (2-amino-5-methylpyridin-4-yl) methanol (70 mg, 0.51 mmol) in DCM (2 mL) at 0° C. were added DMF (4 mg, 0.051 mmol), SOCl₂ (60 mg, 0.51 mmol) and the reaction mixture was stirred at the same temperature for 1 h. The reaction mixture was concentrated under reduced pressure and the residue was purified by silica gel chromatography to afford the title compound (50 mg, 63%). LCMS (m/z): 157.2 [M+H]⁺.

Step 4: Synthesis of 5-(4-((2-amino-5-methylpyridin-4-yl) methyl) piperazin-1-yl)-N,6-dimethylpicolinamide: To a stirred solution of 4-(chloromethyl)-5-methylpyridin-2-amine (50 mg, 0.32 mmol) in CH₃CN (2 mL) were added N,6-dimethyl-5-(piperazin-1-yl)picolinamide dihydrochloride (98 mg, 0.32 mmol), DIPEA (124 mg, 0.96 mol), KI (53 mg, 0.32 mmol) and the reaction mixture was stirred at 25° C. for 18 h. The reaction mixture was concentrated under reduced pressure and the residue was purified by silica gel chromatography to afford the title compound (60 mg, 53%). LCMS (m/z): 355.3 [M+H]⁺.

Step 5: Synthesis of 5-(4-((2-(3-ethylureido)-5-methylpyridin-4-yl) methyl) piperazin-1-yl)-N,6-dimethylpicolinamide formate: To a stirred solution of 5-(4-((2-amino-5-methylpyridin-4-yl)methyl)piperazin-1-yl)-N,6-dimethylpicolinamide (60 mg, 0.17 mmol) in DMF (2 mL) were added ethyl isocyanate (12 mg, 0.17 mmol), Cs₂CO₃ (111 mg, 0.34 mmol) and the reaction mixture was stirred at 80° C. for 4 h. The reaction mixture was concentrated under reduced pressure and the residue was purified by silica gel chromatography to afford the crude product which was purified by Prep-HPLC to afford the title compound (11 mg, 14%). LCMS (m/z): 426.1 [M+H]¹; ¹HNMR (400 MHz, DMSO-d₆) δ 9.03 (s, 1H), 8.44 (q, J=4.5 Hz, 1H), 8.17 (s, 2H), 7.94 (s, 1H), 7.80 (d, 0.1=8.2 Hz, 1H), 7.48 (d, 1=8.3 Hz, 1H), 7.35 (s, 1H), 3.47 (s, 2H), 3.21-3.14 (m, 2H), 2.99-2.90 (m, 4H), 2.80 (d, J=4.9 Hz, 3H), 2.64-2.53 (m, 4H), 2.49 (s, 3H), 2.20 (s, 3H), 1.08 (t, J=7.2 Hz, 3H).

### Example S-24: Synthesis of 5-(4-((6-(3-ethylureido) pyrimidin-4-yl) methyl) piperazin-1-yl)-N,6-dimethylpicolinamide formate (Compound 230)

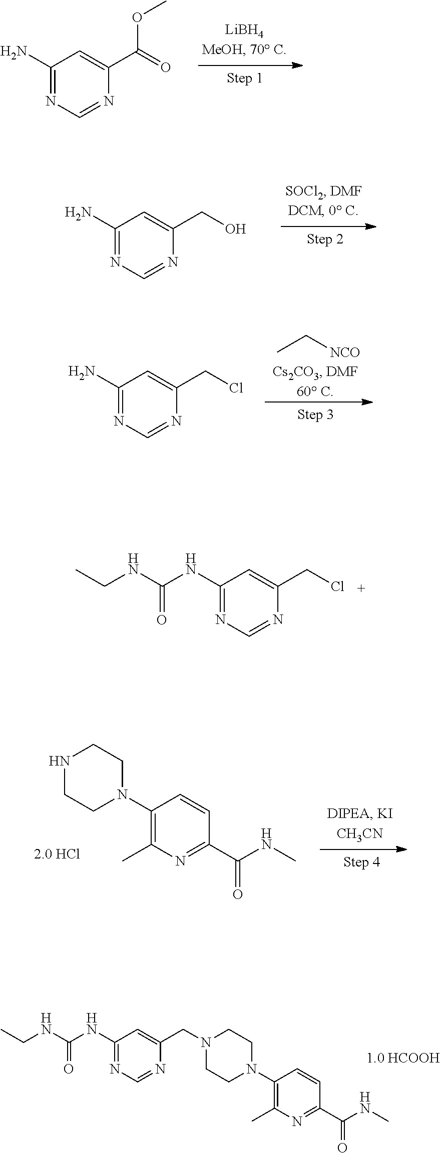

Step 1: Synthesis of (6-aminopyrimidin-4-yl) methanol: To a stirred solution of methyl 6-aminopyrimidine-4-carboxylate (500 mg, 3.26 mmol) in MeOH (10 mL) was added LiBH₄ (213 mg, 9.80 mmol) and the reaction mixture was stirred at 70° C. for 18 h. The reaction mixture was quenched with aqueous NH₄Cl to pH 7 and extracted with MeOH:DCM (1:10, 20 mL×2). The combined organic layers were washed with brine, dried over Na₂SO₄, filtered and the filtrate was concentrated under reduced pressure. The residue was purified by silica gel chromatography to afford the title compound (370 mg, 91%). LCMS (m/z): 126.2 [M+H]⁺.

Step 2: Synthesis of 6-(chloromethyl) pyrimidin-4-amine: To a stirred solution of (6-aminopyrimidin-4-yl) methanol (370 mg, 2.96 mmol) in DCM (10 mL) at 0° C. were added DMF (22 mg, 0.30 mmol) and SOCl₂ (352 mg, 2.96 mmol) and the reaction mixture was stirred at 0° C. for 1 h. The reaction mixture was quenched with aqueous NaHCO₃ to pH 8 and extracted with MeOH:DCM (1:10, 10 mL×2). The combined organic layers were washed with brine, dried over Na₂SO₄, filtered and the filtrate was concentrated under reduced pressure. The residue was purified by silica gel chromatography to afford the title compound (300 mg, 71%). LCMS (m/z): 144.2 [M+H]⁺.

Step 3: Synthesis of 1-(6-(chloromethyl) pyrimidin-4-yl)-3-ethylurea: To a stirred solution of 6-(chloromethyl) pyrimidin-4-amine (150 mg, 1.04 mmol) in DMF (2 mL) were added ethyl isocyanate (74 mg, 1.04 mmol) and CS₂CO₃ (681 mg, 2.09 mmol) and the reaction mixture was stirred at 60° C. for 4 h. The reaction was quenched with aqueous NH₄Cl to pH 7 and extracted with MeOH:DCM (1:10, 5 mL×4). The combined organic layers were washed with brine, dried over Na₂SO₄, filtered and the filtrate was concentrated under reduced pressure. The residue was purified by silica gel chromatography to afford the title compound (80 mg, 36%). LCMS (m/z): 215.1 [M+H]⁺.

Step 4: Synthesis of 5-(4-((6-(3-ethylureido) pyrimidin-4-yl) methyl) piperazin-1-yl)-N,6-dimethylpicolinamide formate: To a stirred solution of 1-(6-(chloromethyl) pyrimidin-4-yl)-3-ethylurea (70 mg, 0.33 mmol) in CH₃CN (2 mL) were added N,6-dimethyl-5-(piperazin-1-yl)picolinamide dihydrochloride (101 mg, 0.33 mmol), DIPEA (127 mg, 0.98 mmol), KI (55 mg, 0.33 mmol) and the reaction mixture was stirred at 25° C. for 18 h. The reaction mixture was concentrated under reduced pressure and the residue was purified by silica gel chromatography to afford the crude product which was purified by Prep-HPLC to afford the title compound (4 mg, 3%). LCMS (m/z): 413.1 [M+H]⁺; ¹HNMR (400 MHz, DMSO-d₆) δ 9.50 (s, 1H), 8.59 (d, J=0.9 Hz, 1H), 8.39 (q, J=4.7 Hz, 1H), 8.20 (s, 1H), 7.76 (d, J=8.2 Hz, 1H), 7.64 (s, 2H), 7.46 (d, J=8.3 Hz, 1H), 3.54 (s, 2H), 3.16-3.10 (m, 2H), 2.96-2.89 (m, 4H), 2.76 (d, J=4.9 Hz, 3H), 2.65-2.54 (m, 4H), 2.45 (s, 3H), 1.04 (t, J=7.2 Hz, 3H).

### Example S-25: Synthesis of 5-(4-((2-(3-(2-fluoroethyl)ureido)pyridin-4-yl)methyl)piperazin-1-yl)-N,6-dimethylpicolinamide (Compound 261)

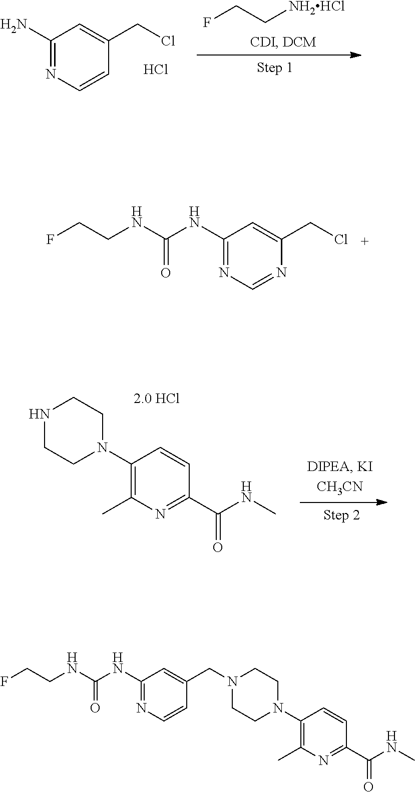

Step 1: Synthesis of 1-(4-(chloromethyl)pyridin-2-yl)-3-(2-fluoroethyl) urea: To a stirred solution of 4-(chloromethyl)pyridin-2-amine hydrochloride (200 mg, 1.12 mmol) in DCM (4 mL) were added 2-fluoroethan-1-amine hydrochloride (133 mg, 1.34 mmol), CDI (217 mg, 1.34 mmol) and the reaction mixture was stirred at 25° C. for 18 h. The reaction mixture was concentrated under reduced pressure and the residue was purified by silica gel chromatography to afford the title compound (80 mg, 31%). LCMS (m/z): 232.2 [M+H]⁺.

Step 2: Synthesis of 5-(4-((2-(3-(2-fluoroethyl)ureido)pyridin-4-yl)methyl)piperazin-1-yl)-N,6-dimethylpicolinamide: To a stirred solution of 1-(4-(chloromethyl) pyridin-2-yl)-3-(2-fluoroethyl) urea (70 mg, 0.30 mmol) in CH₃CN (2 mL) were added N,6-dimethyl-5-(piperazin-1-yl)picolinamide dihydrochloride (114 mg, 0.37 mmol), DIPEA (118 mg, 0.91 mmol), KI (50 mg, 0.30 mmol) and the reaction mixture was stirred at 25° C. for 18 h. The reaction mixture was concentrated under reduced pressure and the residue was purified by silica gel chromatography to afford the crude product which was purified by Prep-HPLC to afford the title compound (45 mg, 35%). LCMS (m/z): 430.3 [M+H]⁺; ¹HNMR (400 MHz, CD₃OD) δ 8.37 (d, J=5.6 Hz, 1H), 7.94 (d, J=8.3 Hz, 1H), 7.61 (d, J=8.3 Hz, 1H), 7.55 (s, 1H), 7.27 (dd, J=5.6, 1.3 Hz, 1H), 4.60 (t, J=5.0 Hz, 1H), 4.51-4.40 (m, 3H), 3.65 (t, J=4.9 Hz, 1H), 3.59 (t, J=4.9 Hz, 1H), 3.57-3.39 (m, 4H), 3.32-3.21 (m, 4H), 2.97 (s, 3H), 2.62 (s, 3H).

### Example S-26: Synthesis of 5-(4-((2-(3-(2,2-difluoroethyl)ureido)pyridin-4-yl)methyl)piperazin-1-yl)-N,6-dimethylpicolinamide (Compound 262)

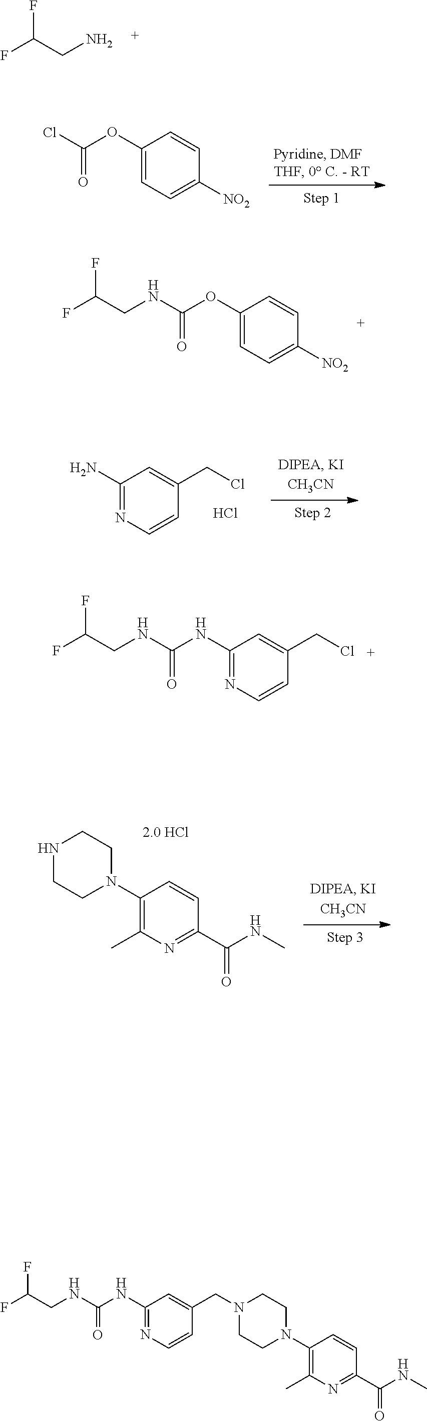

Step 1: Synthesis of 4-nitrophenyl (2,2-difluoroethyl) carbamate: To a stirred solution of 2,2-difluoroethan-1-amine (1 g, 12.34 mmol) in DMF (10 mL) was added pyridine (1.16 g, 14.7 mmol) and the reaction mixture was stirred at 0° C. for 15 min. Then a solution of 4-nitrophenyl chloroformate (2.98 g, 14.78 mmol) in THF (2 mL) was added at 5° C. and the resulting solution was stirred at RT for 18 h. The reaction mixture was quenched with water (50 mL) and extracted with EtOAc (50 mL×2). The combined organic layers were washed with brine, dried over Na₂SO₄, filtered and the filtrate was concentrated under reduced pressure. The residue was purified by silica gel chromatography to afford the title compound (1.5 g, crude). LCMS (m/z): 247.1 [M+H]⁺.

Step 2: Synthesis of 1-(4-(chloromethyl) pyridin-2-yl)-3-(2,2-difluoroethyl) urea: To a stirred solution of 4-nitrophenyl (2,2-difluoroethyl)carbamate (1.3 g, crude) in CH₃CN (20 mL) were added 4-(chloromethyl)pyridin-2-amine hydrochloride (0.38 g, 2.12 mmol), DIPEA (2.05 g, 15.86 mmol), KI (0.88 g, 5.31 mmol) and the reaction mixture was stirred at 25° C. for 18 h. The reaction mixture was concentrated under reduced pressure and the residue was purified by silica gel chromatography to afford the title compound (500 mg, crude). LCMS (m/z): 250.1 [M+H]⁺.

Step 3: Synthesis of 5-(4-((2-(3-(2,2-difluoroethyl) ureido) pyridin-4-yl) methyl) piperazin-1-yl)-N,6-dimethylpicolinamide: To a stirred solution of 1-(4-(chloromethyl) pyridin-2-yl)-3-(2,2-difluoroethyl) urea (500 mg, crude) in CH₃CN (10 mL) were added N,6-dimethyl-5-(piperazin-1-yl)picolinamide dihydrochloride (184 mg, 0.60 mmol), DIPEA (233 mg, 1.80 mmol), KI (100 mg, 0.60 mmol) and the reaction mixture was stirred at 25° C. for 18 h. The reaction mixture was concentrated under reduced pressure and the residue was purified by silica gel chromatography to afford the crude product which was purified by Prep-HPLC to afford the title compound (12.4 mg, 0.3%). LCMS (m/z): 448.3 [M+H]⁺; ¹HNMR (400 MHz, CD₃OD) δ 8.27 (d, 0.1=5.2 Hz, 1H), 7.90 (d, 1=8.3 Hz, 1H), 7.54 (d, J=8.3 Hz, 1H), 7.35 (s, 1H), 7.12 (d, 1=5.2 Hz, 1H), 5.99 (tt, J=56.1, 3.8 Hz, 1H), 4.00 (s, 2H), 3.71 (td, J=15.3, 3.8 Hz, 2H), 3.23-3.12 (m, 4H), 3.13-3.02 (m, 4H), 2.96 (s, 3H), 2.59 (s, 3H).

### Example S-27: Synthesis of N,6-dimethyl-5-(4-((2-(3-(2,2,2-trifluoroethyl) ureido)pyridin-4-yl)methyl)piperazin-1-y) picolinamide trifluoroacetate (Compound 50)

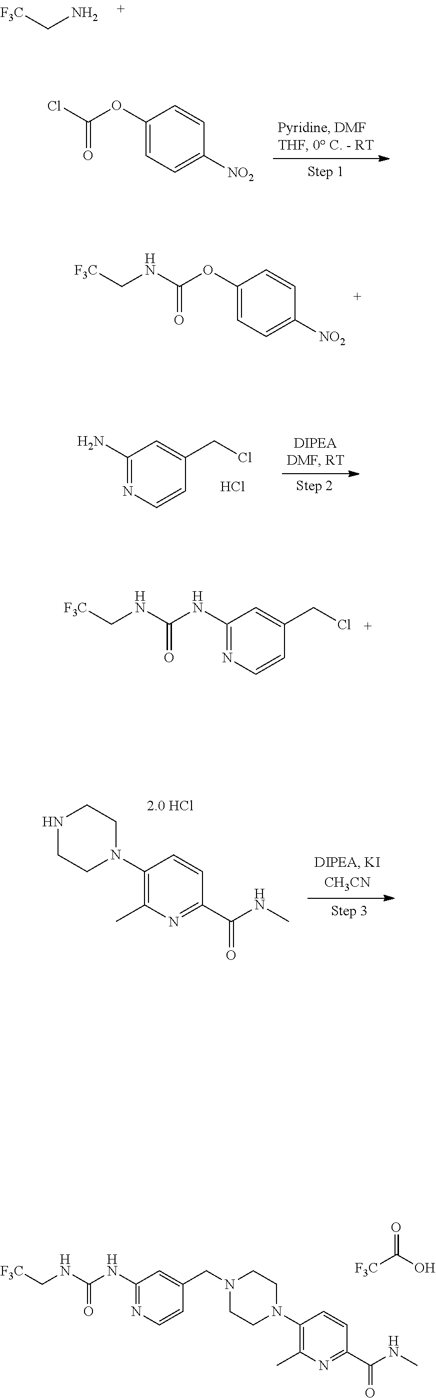

Step 1: Synthesis of 4-nitrophenyl (2,2,2-trifluoroethyl) carbamate: To a stirred solution of 4-nitrophenyl chloroformate (2.42 g, 12 mmol) in DMF (10 mL) at 0° C. was added pyridine (0.95 g, 12 mmol) and the reaction was stirred at 0° C. for 1 h. Then a solution of 2,2,2-trifluoroethan-1-amine (991 mg, 10 mmol) in THF (5 mL) was added and the resulting solution was stirred at RT for 18 h. The reaction mixture was quenched with water (50 mL) and extracted with EtOAc (50 mL×2). The combined organic layers were washed with brine, dried over Na₂SO₄, filtered and the filtrate was concentrated under reduced pressure. The residue was purified by silica gel chromatography to afford the title compound (1.3 g, crude). LCMS (m/z): 265.1 [M+H]⁺.

Step 2: Synthesis of 1-(4-(chloromethyl) pyridin-2-yl)-3-(2,2,2-trifluoroethyl) urea: To a stirred solution of 4-nitrophenyl (2,2,2-trifluoroethyl) carbamate (1.2 g, crude) in DMF (10 mL) were added 4-(chloromethyl) pyridin-2-amine hydrochloride (0.48 g, 2.68 mmol), DIPEA (1.05 g, 8.12 mmol) and the reaction mixture was stirred at 25° C. for 18 h. The reaction mixture was quenched with water and extracted with EtOAc (50 mL×2). The combined organic layers were washed with brine, dried over Na₂SO₄, filtered and the filtrate was concentrated under reduced pressure. The residue was purified by silica gel chromatography to afford the title compound (0.6 g, crude). LCMS (m/z): 268.1 [M+H]⁺.

Step 3: Synthesis of N,6-dimethyl-5-(4-((2-(3-(2,2,2-trifluoroethyl) ureido) pyridin-4-yl)methyl)piperazin-1-yl)picolinamide trifluoroacetate: To a stirred solution of 1-(4-(chloromethyl) pyridin-2-yl)-3-(2,2,2-trifluoroethyl) urea (600 mg, crude) in CH₃CN (10 mL) were added N,6-dimethyl-5-(piperazin-1-yl)picolinamide dihydrochloride (412 mg, 1.34 mmol), DIPEA (520 mg, 4.02 mmol), KI (222 mg, 1.34 mmol and the reaction mixture was stirred at 25° C. for 18 h. The reaction mixture was quenched with water, extracted with EtOAc (20 mL×2). The combined organic layers were washed with brine, dried over Na₂SO₄, filtered and the filtrate was concentrated under reduced pressure. The residue was purified by silica gel chromatography to afford the crude product which was purified by Prep-HPLC to afford the title compound (15 mg, 2%). LCMS (m/z): 466.3 [M+H]⁺; ¹HNMR (400 MHz, CD₃OD) δ 8.39 (d, J=5.4 Hz, 1H), 7.94 (d, J=8.3 Hz, 1H), 7.65-7.58 (m, 2H), 7.28 (dd, J=5.5, 1.4 Hz, 1H), 4.49 (s, 2H), 4.04 (q, J=9.3 Hz, 2H), 3.62-3.48 (m, 4H), 3.33-3.29 (m, 4H), 2.97 (s, 3H), 2.62 (s, 3H).

### Example S-28: Synthesis of 5-(4-((6-(3-ethylureido) pyrimidin-4-yl) methyl) piperazin-1-yl)-6-fluoro-N-methylpicolinamide formate (Compound 229)

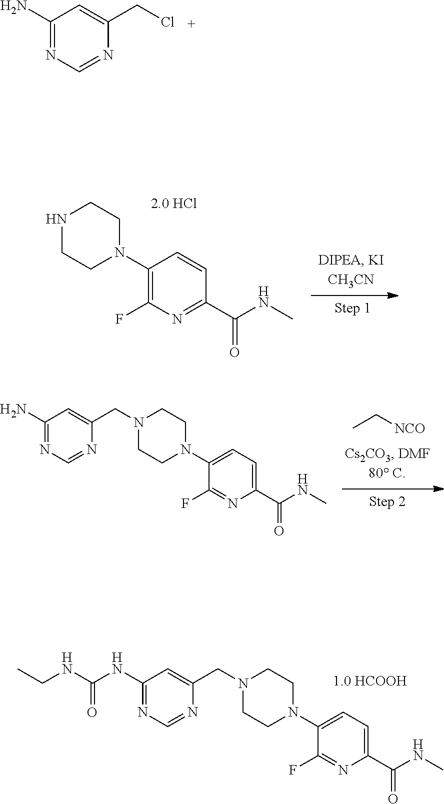

Step 1: Synthesis of 5-(4-((6-aminopyrimidin-4-yl) methyl) piperazin-1-yl)-6-fluoro-N-methylpicolinamide: To a stirred solution of 6-(chloromethyl) pyrimidin-4-amine (160 mg, 1.11 mmol) in CH₃CN (5 mL) were added 6-fluoro-N-methyl-5-(piperazin-1-yl) picolinamide dihydrochloride (349 mg, 1.12 mmol), DIPEA (717 mg, 5.55 mmol), KI (184 mg, 1.11 mmol) and the reaction mixture was stirred at 25° C. for 18 h. The reaction mixture was quenched with water and extracted with DCM (5 mL×3). The combined organic layers were washed with brine, dried over Na₂SO₄, filtered and the filtrate was concentrated under reduced pressure. The residue was purified by silica gel chromatography to afford the title compound (70 mg, 18%). LCMS (m/z): 346.0 [M+H]⁺.

Step 2: Synthesis of 5-(4-((6-(3-ethylureido)pyrimidin-4-yl)methyl)piperazin-1-yl)-6-fluoro-N-methylpicolinamide formate: To a stirred solution of 5-(4-((6-aminopyrimidin-4-yl)methyl)piperazin-1-yl)-6-fluoro-N-methylpicolinamide (65 mg, 0.19 mmol) in DMF (1 mL) were added ethyl isocyanate (20 mg, 0.28 mmol), Cs₂CO₃ (124 mg, 0.38 mmol) and the reaction mixture was stirred at 80° C. for 5 h. The reaction mixture was concentrated under reduced pressure and the residue was purified by C₁₈ chromatography with water/CH₃CN to afford the crude product which was purified by Prep-HPLC to afford the title compound (4 mg, 5%). LCMS (m/z): 417.3 [M+H]⁺; ¹HNMR (400 MHz, CD₃OD) δ 8.58 (s, 1H), 8.07 (s, 1H), 7.80 (dd, J=8.0, 0.8 Hz, 1H), 7.43 (dd, J=10.2, 8.2 Hz, 1H), 7.35 (s, 1H), 3.60 (s, 2H), 3.28-3.21 (m, 6H), 2.81 (s, 3H), 2.68 (s, 4H), 1.11 (t, J=7.2 Hz, 3H).

### Example S-29: Synthesis of 5-(4-((2-(3-ethylureido)-3-fluoropyridin-4-yl) methyl) piperazin-1-yl)-6-fluoro-N-methylpicolinamide (Compound 81)

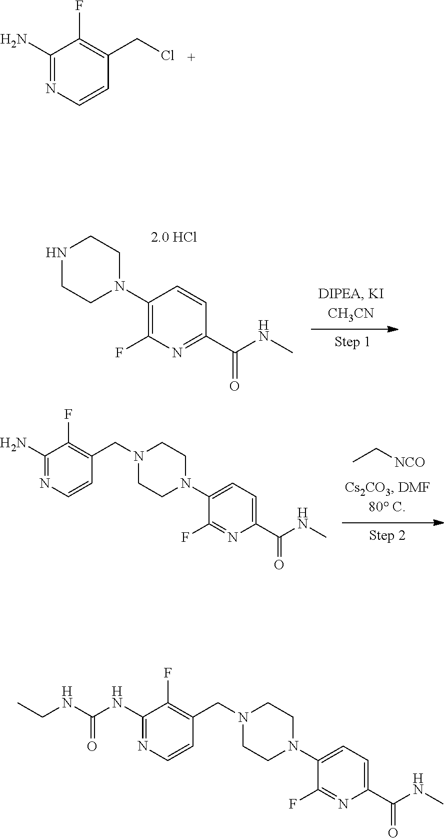

Step 1: Synthesis of 5-(4-((2-amino-3-fluoropyridin-4-yl)methyl)piperazin-1-yl)-6-fluoro-N-methylpicolinamide: To a stirred solution of 4-(chloromethyl)-3-fluoropyridin-2-amine (180 mg, 1.12 mmol) in CH₃CN (5 mL) were added 6-fluoro-N-methyl-5-(piperazin-1-yl) picolinamide dihydrochloride (349 mg, 1.12 mmol), DIPEA (724 mg, 5.6 mmol), KI (186 mg, 1.12 mmol) and the reaction mixture was stirred at 25° C. for 18 h. The reaction mixture was concentrated under reduced pressure and the residue was purified by silica gel chromatography to afford the title compound (120 mg, 30%). LCMS (m/z): 363.3 [M+H]⁺.

Step 2: Synthesis of 5-(4-((2-(3-ethylureido)-3-fluoropyridin-4-yl)methyl)piperazin-1-yl)-6-fluoro-N-methylpicolinamide: To a stirred solution of 5-(4-((2-amino-3-fluoropyridin-4-yl) methyl) piperazin-1-yl)-6-fluoro-N-methylpicolinamide (100 mg, 0.28 mmol) in DMF (2 mL) was added ethyl isocyanate (24 mg, 0.34 mmol), Cs₂CO₃ (179 mg, 0.55 mmol) and the reaction mixture was stirred at 80° C. for 4 h. The reaction mixture was concentrated under reduced pressure and the residue was purified by Prep-HPLC to afford the title compound (19 mg, 16%). LCMS (m/z): 434.3 [M+H]⁺; ¹H NMR (400 MHz, CD₃OD) δ 8.04 (d, 0.1=5.2 Hz, 1H), 7.91 (dd, J=8.1, 1.2 Hz, 1H), 7.53 (dd, J=10.2, 8.1 Hz, 1H), 7.16 (t, J=5.0 Hz, 1H), 3.73 (s, 2H), 3.39 (q, J=7.2 Hz, 2H), 3.30-3.25 (m, 4H), 2.93 (s, 3H), 2.76-2.69 (m, 4H), 1.24 (t, 0.1=7.2 Hz, 3H).

### Example S-30: Synthesis of 1-(6-(1H-imidazol-2-yl)-2-methylpyridin-3-yl)piperazine trifluoroacetate

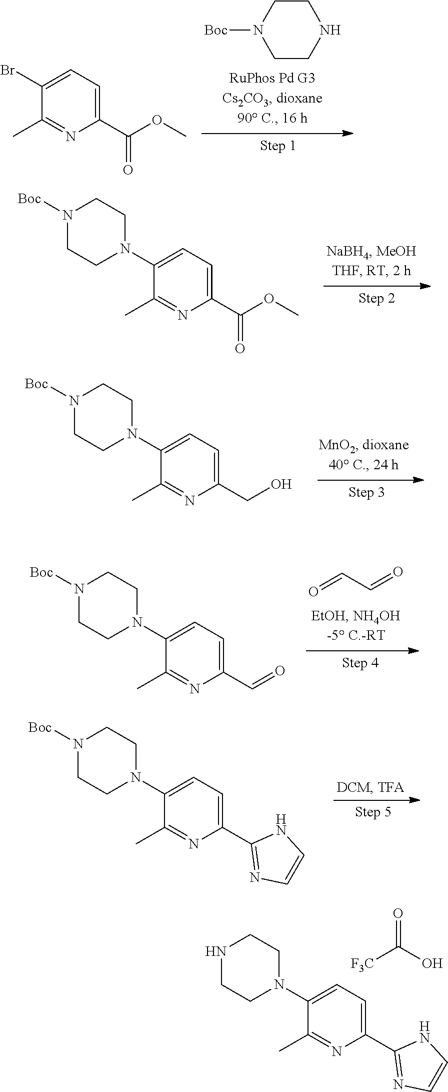

Step 1: Synthesis of tert-butyl 4-(6-(methoxycarbonyl)-2-methylpyridin-3-yl) piperazine-1-carboxylate: A mixture of methyl 5-bromo-6-methylpicolinate (5 g, 21.7 mmol), tert-butyl piperazine-1-carboxylate (4.84 g, 26.0 mmol), Cs₂CO₃ (14.1 g, 43.4 mmol) and RuPhosPdG3 (1.8 g, 2.2 mmol) in 1,4-dioxane (50 mL) was stirred at 90° C. for 16 h under N2 atmosphere. The reaction was quenched with water (200 mL) and extracted with EtOAc (200 mL×3). The combined organic layers were washed with water and brine, dried over Na₂SO₄, filtered, and the filtrate was concentrated under reduced pressure. The crude residue was purified by silica gel chromatography to afford the title compound (5 g, 69%). LCMS (m/z): 336.3 [M+H]⁺.

Step 2: Synthesis of tert-butyl 4-(6-(hydroxymethyl)-2-methylpyridin-3-yl) piperazine-1-carboxylate: To a solution of tert-butyl 4-(6-(methoxycarbonyl)-2-methylpyridin-3-yl)piperazine-1-carboxylate (2 g, 5.96 mmol) in MeOH:THF (4:1, 10 mL) was added NaBH₄ (1.35 g, 35.67 mmol) and the reaction was stirred at RT for 2 h. The reaction was quenched with water (100 mL) and extracted with EtOAc (100 mL×3). The combined organic layers were washed with water and brine, dried over Na₂SO₄, filtered, and the filtrate was concentrated under reduced pressure. The residue was purified by silica gel chromatography to afford the title compound (1.6 g, 87%). LCMS (m/z): 308.1 [M+H]⁺.

Step 3: Synthesis of tert-butyl 4-(6-formyl-2-methylpyridin-3-yl)piperazine-1-carboxylate: To a solution of tert-butyl 4-(6-(hydroxymethyl)-2-methylpyridin-3-yl)piperazine-1-carboxylate (1.5 g, 4.88 mmol) in dioxane (20 mL) was added MnO₂ (2.11 g, 24.32 mmol) and the resulting reaction was stirred at 40° C. for 24 h. The solid was filtered out and the filtrate was concentrated under reduced pressure. The crude residue was purified by silica gel chromatography to afford the title compound (1.35 g, 91%). LCMS (m/z): 306.2 [M+H]⁺.

Step 4: Synthesis of tert-butyl 4-(6-(1H-imidazol-2-yl)-2-methylpyridin-3-yl) piperazine-1-carboxylate: To an ice-cold solution of the tert-butyl 4-(6-formyl-2-methylpyridin-3-yl)piperazine-1-carboxylate (100 mg, 0.33 mmol) in ethanol (4 mL) was added a solution of 40% aqueous glyoxal (59 mg, 0.41 mmol) followed by dropwise addition of NH₄OH (1 mL) at −5° C. and the solution was stirred at −5° C. for 1 h. The reaction was warmed to RT and stirred at the same temperature for 16 h. The solvent was removed in vacuo. The crude product was purified by silica gel chromatography to afford the title compound (90 mg, 80%). LCMS (m/z): 235.1 [M+H]⁺.

Step 5: Synthesis of 1-(6-(1H-imidazol-2-yl)-2-methylpyridin-3-yl)piperazine trifluoroacetate: To a solution of tert-butyl 4-(6-(1H-imidazol-2-yl)-2-methylpyridin-3-yl)piperazine-1-carboxylate (70 mg, 0.20 mmol) in DCM (3 mL) was added TFA (1 mL). The resulting solution was stirred at RT for 1 h. The reaction was concentrated in vacuo to afford the title compound (70 mg, 97%). LCMS (m/z): 244.0 [M+H]⁺.

### Example S-31: Synthesis of N-(6-methyl-5-(piperazin-1-yl)pyridin-2-yl)acetamide trifluoroacetate

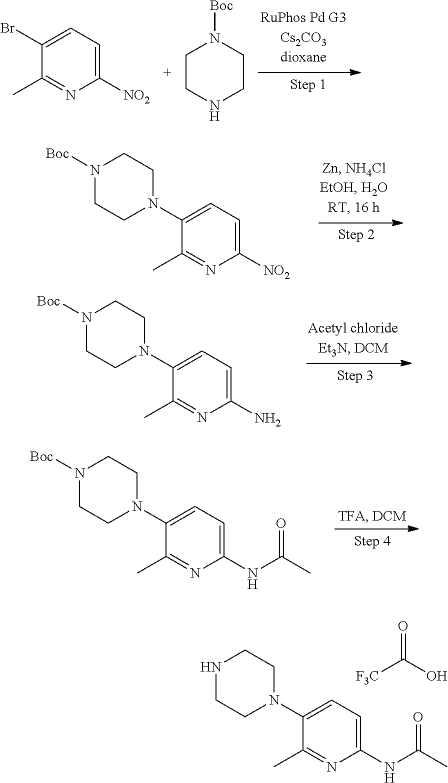

Step 1: Synthesis of tert-butyl 4-(2-methyl-6-nitropyridin-3-yl)piperazine-1-carboxylate: A mixture of 3-bromo-2-methyl-6-nitropyridine (500 mg, 2.30 mmol), tert-butyl piperazine-1-carboxylate (514 mg, 2.76 mmol), CS₂CO₃ (1.5 g, 4.61 mmol) and RuPhosPdG3 (193 mg, 0.23 mmol) in 1,4-dioxane (5 mL) was stirred at 80° C. for 16 h under N2 atmosphere. The reaction was quenched with water (50 mL) and extracted with EtOAc (50 mL×3). The combined organic layers were washed with water and brine, dried over Na₂SO₄, filtered, and the filtrate was concentrated under reduced pressure. The crude residue was purified by silica gel chromatography to afford the title compound (350 mg, 47%). LCMS (m/z): 322.9 [M+H]⁺.

Step 2: Synthesis of tert-butyl 4-(6-amino-2-methylpyridin-3-yl)piperazine-1-carboxylate: A mixture of tert-butyl 4-(2-methyl-6-nitropyridin-3-yl)piperazine-i-carboxylate (250 mg, 0.78 mmol), Zn (252 mg, 3.87 mmol) and NH₄Cl (414 mg, 7.73 mmol) in EtOH:H₂O (5:1, 6 mL) was stirred at RT for 16 h. The solid was filtered and the filtrate was concentrated under reduced pressure. The residue was quenched with water (50 mL) and extracted with EtOAc (50 mL×3). The combined organic layers were washed with water and brine, dried over Na₂SO₄, filtered, and the filtrate was concentrated under reduced pressure. The residue was purified by silica gel chromatography to afford the title compound (200 mg, 88%). LCMS (m/z): 293.0 [M+H]⁺.

Step 3: Synthesis of tert-butyl 4-(6-acetamido-2-methylpyridin-3-yl)piperazine-1-carboxylate: To a solution of tert-butyl 4-(6-amino-2-methylpyridin-3-yl)piperazine-1-carboxylate (180 mg, 0.62 mmol) in dry DCM (3 mL) were added acetyl chloride (53 mg, 0.67 mmol) and Et₃N (124 mg, 1.23 mmol) and the resulting solution was stirred at RT for 1 h. Then the solvent was removed in vacuo and the crude product was purified by silica gel chromatography to afford the title compound (150 mg, 73%). LCMS (m/z): 335.3 [M+H]⁺.

Step 4: Synthesis of N-(6-methyl-5-(piperazin-1-yl)pyridin-2-yl)acetamide trifluoroacetate: To a solution of tert-butyl 4-(6-acetamido-2-methylpyridin-3-yl)piperazine-1-carboxylate (140 mg, 0.42 mmol) in DCM (3 mL) was added TFA (1 mL) and the resulting solution was stirred at RT for 1 h. The reaction mixture was concentrated in vacuo to afford the title compound (80 mg, 55%). LCMS (m/z): 235.1 [M+H]⁺.

### Example S-32: Synthesis of 1-[2-methyl-6-(pyrazol-1-yl)pyridin-3-yl]piperazine

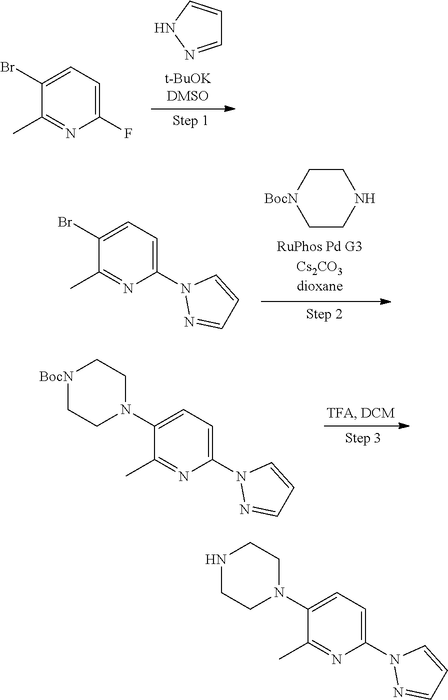

Step 1: Synthesis of 3-bromo-2-methyl-6-(pyrazol-1-yl)pyridine: To a solution of 3-bromo-6-fluoro-2-methylpyridine (500 mg, 2.63 mmol) in DMSO (5 mL) were added t-BuOK (590 mg, 5.26 mmol), 1H-pyrazole (215 mg, 3.16 mmol) and the resulting solution was stirred at 100° C. for 16 h before it was then quenched with water (30 mL) and extracted with EtOAc (30 mL×3). The combined organic layers were washed with water and brine, dried over sodium sulfate, filtered, the filtrate was concentrated, and the residue was purified by silica gel chromatography to afford desired compound (100 mg, 16%). LCMS (m/z): 238.0 [M+H]⁺.

Step 2: Synthesis of tert-butyl 4-(2-methyl-6-(1H-pyrazol-1-yl)pyridin-3-yl)piperazine-1-carboxylate: A mixture of 3-bromo-2-methyl-6-(pyrazol-1-yl)pyridine (100 mg, 0.42 mmol), tert-butyl piperazine-1-carboxylate (94 mg, 0.50 mmol), Cs₂C₀. (274 mg, 0.84 mmol) and RuPhosPdG3 (33.5 mg, 0.04 mmol) in 1,4-dioxane (4 mL) was stirred at 80° C. for 16 h under nitrogen atmosphere. The reaction was then quenched with water (10 mL) and extracted with EtOAc (10 mL×3). The organic layer was washed with water and brine, dried over sodium sulfate, filtered, the filtrate was concentrated, and the residue was purified by silica gel chromatography to afford desired compound (50 mg, 35%). LCMS (m/z): 344.0 [M+H]⁺.

Step 3: Synthesis of 1-[2-methyl-6-(pyrazol-1-yl)pyridin-3-yl]piperazine: To a solution of tert-butyl 4-(2-methyl-6-(1H-pyrazol-1-yl)pyridin-3-yl)piperazine-1-carboxylate (50 mg, 0.15 mmol) in DCM (1.5 mL) was added TFA (0.5 mL,) and the resulting solution was stirred at RT for 1 h. The solvent was removed in vacuo. The residue was dissolved in water, neutralized with saturated aqueous NaHCO₃ and extracted with DCM. The organic layer was concentrated and purified by silica gel chromatography to afford the title compound (30 mg, 83%). LCMS (m/z): 244.1 [M+H]⁺.

### Example S-33: Synthesis of 1-(4-((4-(6-(JH-imidazol-2-yl)-2-methylpyridin-3-yl)piperazin-1-yl) methyl)pyridin-2-yl)-3-ethylurea formate (Compound 272)

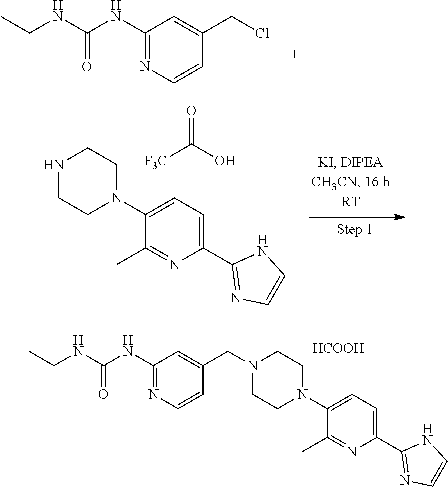

Step 1: Synthesis of 1-(4-((4-(6-(1H-imidazol-2-yl)-2-methylpyridin-3-yl)piperazin-1-yl)methyl)pyridin-2-yl)-3-ethylurea formate: A mixture of 1-(4-(chloromethyl)pyridin-2-yl)-3-ethylurea (30 mg, 0.14 mmol), 1-(6-(1H-imidazol-2-yl)-2-methylpyridin-3-yl)piperazine trifluoroacetate (60 mg, crude), KI (35 mg, 0.21 mmol) and DIPEA (181 mg, 1.40 mmol) in CH₃CN (3 mL) was stirred at RT for 16 h. The reaction mixture was quenched with water and extracted with DCM (10 mL×3). The combined organic layers were washed with brine, dried over Na₂SO₄, filtered, and the filtrate was concentrated under reduced pressure. The residue was purified by prep-HPLC to afford the title compound (8 mg, 12%). LCMS (m/z): 421.1 [M+H]⁺; ¹H NMR (400 MHz, CD₃OD) δ 8.20-8.16 (m, 2H), 7.85 (d, J=8.4 Hz, 1H), 7.56 (d, J=8.4 Hz, 1H), 7.34-7.29 (m, 2H), 7.18 (s, 1H), 7.03 (d, J=5.3 Hz, 1H), 3.67 (s, 2H), 3.38-3.34 (m, 2H), 3.12-3.06 (m, 4H), 2.80-2.72 (m, 4H), 2.61 (s, 3H), 1.23 (t, J=7.2 Hz, 3H).

### Example S-34: Synthesis of N-(5-(4-((2-(3-ethylureido)pyridin-4-yl)methyl)piperazin-1-yl)-6-methylpyridin-2-yl)acetamide (Compound 269)

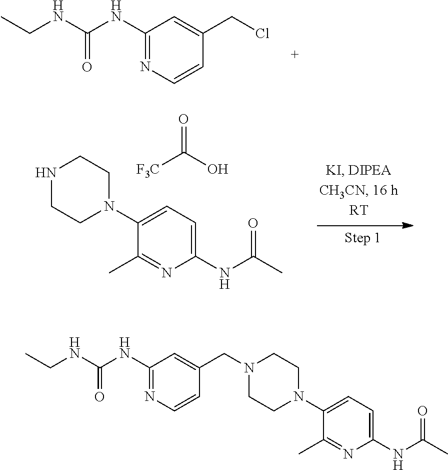

Step 1: Synthesis of N-(5-(4-((2-(3-ethylureido)pyridin-4-yl)methyl)piperazin-1-yl)-6-methylpyridin-2-yl)acetamide: A mixture of 1-(4-(chloromethyl)pyridin-2-yl)-3-ethylurea (50 mg, 0.23 mmol), N-(6-methyl-5-(piperazin-1-yl)pyridin-2-yl)acetamide trifluoroacetate (98 mg, 0.28 mmol), KI (58 mg, 0.35 mmol) and DIPEA (302 mg, 2.34 mmol) in CH₃CN (3 mL) was stirred at RT for 16 h. The reaction was concentrated under reduced pressure and the residue was purified by prep-HPLC to afford the title compound (17 mg, 18%). LCMS (m/z): 412.2 [M+H]⁺; ¹H NMR (400 MHz, CD₃OD) δ 8.17 (d, 1=5.3 Hz, 1H), 7.84 (d, J=8.6 Hz, 1H), 7.49 (d, 0.1=8.7 Hz, 1H), 7.14 (s, 1H), 7.02 (d, J=5.2 Hz, 1H), 3.63 (s, 2H), 3.40-3.34 (m, 2H), 3.01-2.93 (m, 4H), 2.75-2.65 (m, 4H), 2.44 (s, 3H), 2.15 (s, 3H), 1.23 (t, J=7.2 Hz, 3H).

### Example S-35: Synthesis of 1-ethyl-3-(4-((4-(2-methyl-6-(1H-pyrazol-1-yl)pyridin-3-yl)piperazin-1-yl)methyl)pyridin-2-yl)urea (Compound 24)

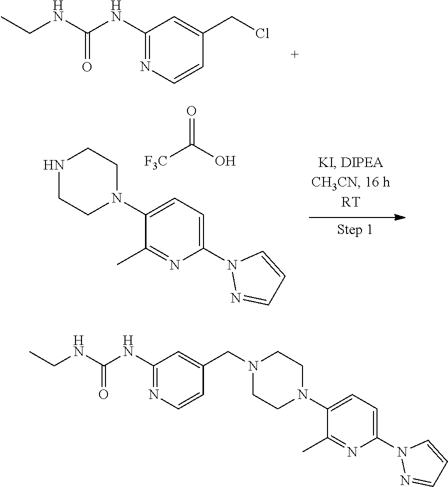

Step 1: Synthesis of 1-ethyl-3-(4-((4-(2-methyl-6-(1H-pyrazol-1-yl)pyridin-3-y) piperazin-1-yl)methyl)pyridin-2-yl)urea: A mixture of 1-(4-(chloromethyl)pyridin-2-yl)-3-ethylurea (50 mg, 0.23 mmol), 1-(2-methyl-6-(1H-pyrazol-1-yl)pyridin-3-yl)piperazine 2,2,2-trifluoroacetate (100 mg, 0.28 mmol), KI (58 mg, 0.35 mmol) and DIPEA (302 mg, 2.34 mmol) in CH₃CN (3 mL) was stirred at RT for 16 h. The reaction was concentrated under reduced pressure and the residue was purified by prep-HPLC to afford the title compound (15 mg, 16%). LCMS (m/z): 421.3 [M+H]⁺; ¹H NMR (400 MHz, CD₃OD) δ 8.48 (d, J=2.5 Hz, 1H), 8.12 (d, J=5.3 Hz, 1H), 7.70-7.63 (m, 2H), 7.58 (d, J=8.6 Hz, 1H), 7.09 (s, 1H), 6.98 (d, J=5.2 Hz, 1H), 6.47 (d, J=1.8 Hz, 1H), 3.57 (s, 2H), 3.34-3.29 (m, 2H), 3.01-2.93 (m, 4H), 2.73-2.58 (m, 4H), 2.52-2.50 (m, 3H), 1.18 (t, J=7.2 Hz, 3H).

### Example S-36: Synthesis of 5-(4-(3-(3-ethylureido)benzyl)piperazin-1-yl)-N,6-dimethyl picolinamide formate (Compound 202)

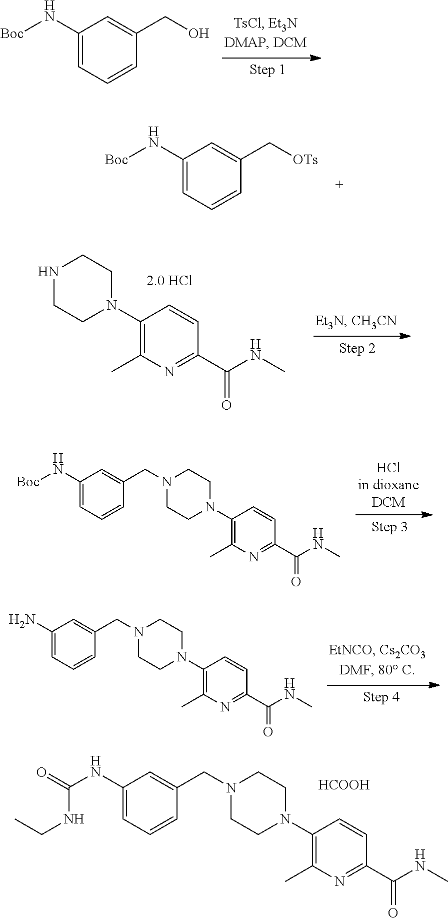

Step 1: Synthesis of 3-((tert-butoxycarbonyl)amino)benzyl 4-methylbenzene sulfonate: To a solution of tert-butyl (3-(hydroxymethyl) phenyl) carbamate (200 mg, 0.9 mmol) in DCM (5 mL) were added TsCl (172 mg, 0.9 mmol), Et-N (273 mg, 2.7 mmol), and DMAP (110 mg, 0.9 mmol) and the reaction was stirred at 25° C. for 18 h. The reaction mixture was quenched with water and extracted with DCM (5 mL×3). The combined organic layers were washed with brine, dried over Na₂SO₄, filtered, and the filtrate was concentrated under reduced pressure. The residue was purified by silica gel chromatography to afford the title compound (160 mg, 47%). LCMS (m/z): 400.2 [M+Na]⁺.

Step 2: Synthesis of tert-butyl (3-((4-(2-methyl-6-(methyl carbamoyl)pyridin-3-yl) piperazin-1-yl)methyl)phenyl)carbamate: To a stirred solution of 3-((tert-butoxycarbonyl) amino)benzyl 4-methylbenzenesulfonate (160 mg, 0.42 mmol) in CH₃CN (4 mL) were added methyl 6-methyl-5-(piperazin-1-yl)picolinate dihydrochloride (132 mg, 0.43 mmol) and Et₃N (128 mg, 1.27 mmol) and the reaction was stirred at 25° C. for 18 h. The reaction mixture was quenched with saturated NH₄Cl and extracted with DCM (4 mL×2). The combined organic layers were washed with brine, dried over Na₂SO₄, filtered, and the filtrate was concentrated under reduced pressure. The residue was purified by silica gel chromatography to afford the title compound (180 mg, 97%). LCMS (m/z): 440.3 [M+H]⁺.

Step 3: Synthesis of 5-(4-(3-aminobenzyl) piperazin-1-yl)-N,6-dimethylpicolinamide: To a stirred solution of tert-butyl(3-((4-(2-methyl-6-(methylcarbamoyl)pyridin-3-yl)piperazin-1-yl)methyl)phenyl)carbamate (180 mg, 0.41 mmol) in DCM (4 mL) was added 4M HCl in dioxane (1 mL) and the reaction was stirred at 25° C. for 6 h. The reaction mixture was quenched with saturated NaHCO₃ to pH 8 and extracted with DCM:MeOH (10:1, 4 mL×3). The combined organic layers were washed with brine, dried over Na₂SO₄, filtered, and the filtrate was concentrated under reduced pressure. The residue was purified by silica gel chromatography to afford the title compound (100 mg, 72%). LCMS (m/z): 430.3 [M+H]⁺.

Step 4: Synthesis of 5-(4-(3-(3-ethylureido)benzyl)piperazin-1-yl)-N,6-dimethyl picolinamide formate: To a stirred solution of 5-(4-(3-aminobenzyl)piperazin-1-yl)-N,6-dimethylpicolinamide (80 mg, 0.24 mmol) in DMF (2 mL) were added ethyl isocyanate (20 mg, 0.28 mmol) and Cs₂CO₃ (153 mg, 0.47 mmol) and the reaction was stirred at 80° C. for 4 h. The reaction mixture was filtered, and the filter cake was washed with MeOH (10 mL×3), and the filtrate was concentrated under reduced pressure. The residue was purified by prep-HPLC to afford the title compound (6 mg, 6%). LCMS (m/z): 411.3 [M+H]⁺; ¹H NMR (400 MHz, CD₃OD) δ 8.32 (s, 1H), 7.85 (d, J=8.3 Hz, 1H), 7.55 (s, 1H), 7.49 (d, J=8.3 Hz, 1H), 7.34-7.20 (m, 2H), 7.04 (d, J=6.5 Hz, 1H), 3.94 (s, 2H), 3.21 (q, J=7.2 Hz, 2H), 3.15-3.08 (m, 4H), 3.08-2.99 (m, 4H), 2.91 (s, 3H), 2.53 (s, 3H), 1.13 (t, J=7.2 Hz, 3H).

### Example S-37. Synthesis of 6-(4-((2-(3-ethylureido)pyridin-4-yl)methyl)piperazin-1-yl)-N,1-dimethyl-2-oxo-1,2-dihydropyridine-3-carboxamide (Compound 30)

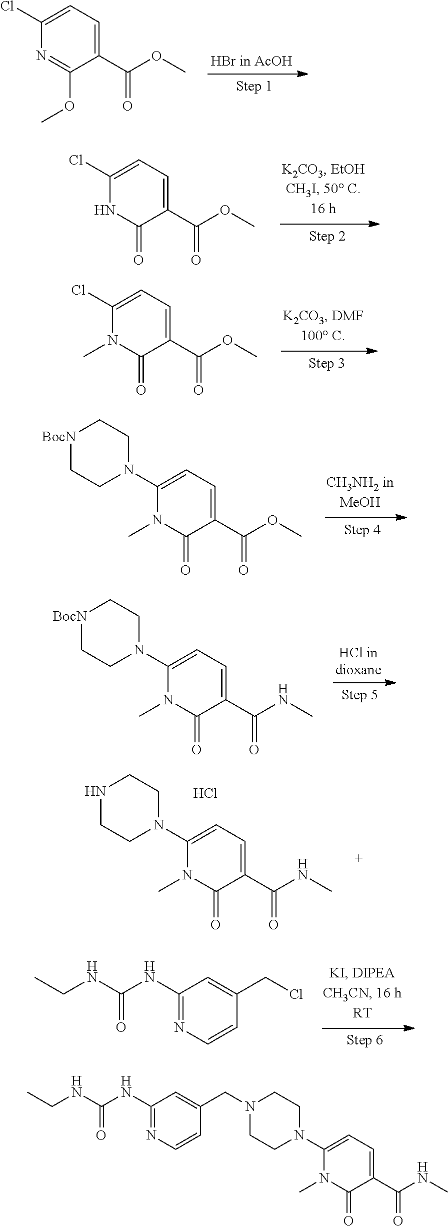

Step 1: Synthesis of methyl 6-chloro-2-oxo-1,2-dihydropyridine-3-carboxylate: Methyl 6-chloro-2-methoxynicotinate (500 mg, 2.48 mmol) was added to a solution of 33% HBr in AcOH (5 mL) at 0° C. and the reaction mixture was stirred at RT for 3 h. The reaction was concentrated under reduced pressure and the residue was neutralized with aqueous NaHCO₃ to pH 4-5 and extracted with EtOAc (50 mL×3). The combined organic layers were washed with water and brine, dried over Na₂SO₄, filtered and the filtrate was concentrated under reduced pressure. The residue was purified by silica gel chromatography to afford the title compound (400 mg, 86%). LCMS (m/z): 187.9 [M+H]⁺.

Step 2: Synthesis of methyl 6-chloro-1-methyl-2-oxo-1,2-dihydropyridine-3-carboxylate: To a solution of methyl 6-chloro-2-oxo-1,2-dihydropyridine-3-carboxylate (400 mg, 2.13 mmol) in dry CH₃CN (5 mL) were added K₂CO₃ (590 mg, 4.26 mmol), iodomethane (605 mg, 4.26 mmol) and the resulting solution was stirred at 50° C. for 16 h in a sealed tube and concentrated under reduced pressure. The residue was purified by silica gel chromatography to afford the title compound (300 mg, 70%). LCMS (m/z): 202.1 [M+H]⁺.

Step 3: Synthesis of tert-butyl 4-(5-(methoxycarbonyl)-1-methyl-6-oxo-1,6-dihydropyridin-2-yl)piperazine-1-carboxylate: To a solution of methyl 6-chloro-1-methyl-2-oxo-1,2-dihydropyridine-3-carboxylate (100 mg, 0.50 mmol) in DMF (5 mL) were added tert-butyl piperazine-1-carboxylate (111 mg, 0.60 mmol), K₂CO₃ (137 mg, 0.99 mmol) and the resulting solution was stirred at 100° C. for 4 h. The reaction mixture was quenched with water (50 mL) and extracted with EtOAc (50 mL×3). The combined organic layers were washed with water and brine, dried over Na₂SO₄, filtered and the filtrate was concentrated under reduced pressure. The residue was purified by silica gel chromatography to afford the title compound (110 mg, 63%). LCMS (m/z): 352.0 [M+H]⁺.

Step 4: Synthesis of tert-butyl 4-(1-methyl-5-(methylcarbamoyl)-6-oxo-1,6-dihydropyridin-2-yl)piperazine-1-carboxylate: To a solution of tert-butyl 4-(5-(methoxycarbonyl)-1-methyl-6-oxo-1,6-dihydropyridin-2-yl)piperazine-1-carboxylate (110 mg, 0.31 mmol) in MeOH (2 mL) was added 33% CH₃NH₂ in MeOH (1 mL) and the resulting solution was stirred at RT for 16 h and concentrated under reduced pressure. The residue was purified by silica gel chromatography to afford the title compound (100 mg, 92%). LCMS (m/z): 351.0 [M+H]⁺.

Step 5: Synthesis of N,1-dimethyl-2-oxo-6-(piperazin-1-yl)-1,2-dihydropyridine-3-carboxamide hydrochloride: To a solution of tert-butyl 4-(1-methyl-5-(methylcarbamoyl)-6-oxo-1,6-dihydropyridin-2-yl) piperazine-1-carboxylate (100 mg, 0.28 mmol) in DCM (3 mL) was added 4M HCl in 1,4-dioxane (1.5 mL) and the reaction mixture was stirred at RT for 3 h. The reaction was concentrated under reduced pressure to afford the title compound (100 mg, crude). LCMS (m/z): 251.0 [M+H]⁴.

Step 6: Synthesis of 6-(4-((2-(3-ethylureido)pyridin-4-yl)methyl)piperazin-1-yl)-N,1-dimethyl-2-oxo-1,2-dihydropyridine-3-carboxamide: A mixture of 1-(4-(chloromethyl) pyridin-2-yl)-3-ethylurea (50 mg, 0.23 mmol), N,1-dimethyl-2-oxo-6-(piperazin-1-yl)-1,2-dihydropyridine-3-carboxamide hydrochloride (80 mg, 0.28 mmol), KI (58 mg, 0.35 mmol), and DIPEA (302 mg, 2.34 mmol) in CH₃CN (3 mL) was stirred at RT for 16 h. The reaction mixture was concentrated under reduced pressure and the residue was purified by prep-HPLC to afford the title compound (15 mg, 15%). LCMS (m/z): 428.2 [M+H]⁺; ¹H NMR (400 MHz, CDCl₃) δ 13.24 (s, 1H), 9.78-9.75 (m, 1H), 8.46 (d, J=8.1 Hz, 1H), 8.10-7.89 (m, 2H), 7.29 (s, 1H), 6.04 (d, J=8.1 Hz, 1H), 3.79 (s, 2H), 3.58 (s, 3H), 3.38-3.30 (m, 2H), 3.18-3.09 (m, 4H), 2.99 (d, J=4.8 Hz, 3H), 2.78-2.68 (m, 4H), 1.23 (t, J=7.2 Hz, 3H).

### Example S-38: Synthesis of 6-cyano-5-(4-((2-(3-ethylureido) pyridin-4-yl) methyl) piperazin-1-yl)-N-methylpicolinamide (Compound 22)

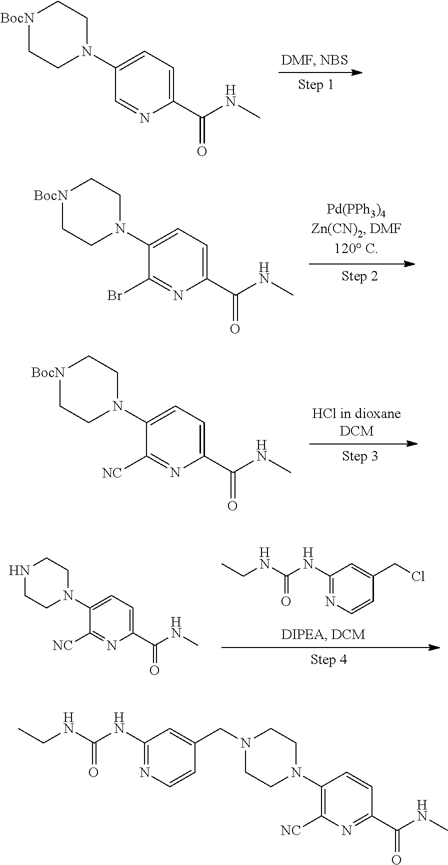 [SMILES: CNC(=O)c1ccc(N2CCN(C(=O)OC(C)(C)C)CC2)cn1 ; CNC(=O)c1ccc(N2CCN(C(=O)OC(C)(C)C)CC2)c(Br)n1 ; CNC(=O)c1ccc(N2CCN(C(=O)OC(C)(C)C)CC2)c(C#N)n1 ; CNC(=O)c1ccc(N2CCNCC2)c(C#N)n1 ; CCNC(=O)Nc1cc(CCl)ccn1 ; CCNC(=O)Nc1cc(CN2CCN(c3ccc(C(=O)NC)nc3C#N)CC2)ccn1]

Step 1: Synthesis of tert-butyl 4-(2-bromo-6-(methyl carbamoyl) pyridin-3-yl) piperazine-1-carboxylate: To a solution of tert-butyl 4-(6-(methyl carbamoyl) pyridin-3-yl) piperazine-1-carboxylate (310 mg, 0.97 mmol) in DMF (4 mL) was added NBS (189 mg, 1.06 mmol) and the reaction was stirred at 25° C. for 18 h. The reaction mixture was quenched with water (4 mL) and extracted with EtOAc (4 mL×2). The combined organic layers were washed with brine, dried over Na₂SO₄, filtered, and the filtrate was concentrated under reduced pressure. The residue was purified by silica gel chromatography to afford the title compound (200 mg, 52%). LCMS (m/z): 399.2 [M+H]⁺.

Step 2: Synthesis of tert-butyl 4-(2-cyano-6-(methylcarbamoyl)pyridin-3-yl)piperazine-1-carboxylate: To a solution of tert-butyl 4-(2-bromo-6-(methylcarbamoyl) pyridin-3-yl)piperazine-1-carboxylate (190 mg, 0.48 mmol) in DMF (4 mL) were added Zn(CN)₂ (167 mg, 1.42 mmol), Pd(PPh₃)₄ (110 mg, 0.095 mmol) and the reaction was stirred at 120° C. for 4 h under N2 atmosphere. The reaction mixture was quenched with water and extracted with EtOAc (10 mL×2). The combined organic layers were washed with brine, dried over Na₂SO₄, filtered, and the filtrate was concentrated under reduced pressure. The residue was purified by silica gel chromatography to afford the title compound (110 mg, 66%). LCMS (m/z): 368.0 [M+Na]⁺.

Step 3: Synthesis of 6-cyano-N-methyl-5-(piperazin-1-yl) picolinamide: To a solution of tert-butyl 4-(2-cyano-6-(methylcarbamoyl)pyridin-3-yl) piperazine-1-carboxylate (110 mg, 0.32 mmol) in DCM (2 mL) was added 1M HCl in dioxane (1 mL) and the reaction was stirred at 25° C. for 4 h. The reaction mixture was quenched with saturated NaHCO₃ to pH 7 and extracted with DCM:MeOH (10:1, 3 mL×3). The combined organic layers were washed with brine, dried over Na₂SO₄, filtered, and the filtrate was concentrated under reduced pressure. The residue was purified by silica gel chromatography to afford the title compound (75 mg, 96%). 246.1 [M+H]⁺.

Step 4: Synthesis of 6-cyano-5-(4-((2-(3-ethylureido) pyridin-4-yl) methyl) piperazin-1-yl)-N-methylpicolinamide: To a solution of 6-cyano-N-methyl-5-(piperazin-1-yl) picolinamide (75 mg, 0.31 mmol) in DCM (2 mL) were added 1-(4-(chloromethyl)pyridin-2-yl)-3-ethylurea (66 mg, 0.31 mmol) and DIPEA (119 mg, 0.92 mmol) and the reaction was stirred at 25° C. for 18 h. The reaction mixture was quenched with water and extracted with DCM:MeOH (10:1, 3 mL×3). The combined organic layers were washed with brine, dried over Na₂SO₄, filtered, and the filtrate was concentrated under reduced pressure. The residue was purified by silica gel chromatography to give the crude product which was purified by prep-HPLC to afford the title compound (64 mg, 49%). LCMS (m/z): 423.3 [M+H]⁺: ¹H NMR (400 MHz, DMSO-d₆) δ 9.14 (s, 1H), 8.58 (d, J=4.7 Hz, 1H), 8.22-8.14 (m, 1H), 8.13-8.08 (m, 2H), 7.75 (d, J=8.9 Hz, 1H), 7.34 (s, 1H), 6.90 (d, J=5.1 Hz, 1H), 3.52 (s, 2H), 3.46-3.35 (m, 4H), 3.24-3.04 (m, 2H), 2.79 (d, 0.1=4.7 Hz, 3H), 2.65-2.53 (m, 4H), 1.09 (t, J=7.2 Hz, 3H).

### Example S-39: Synthesis of 1-ethyl-3-(4-((4-(5-(trifluoromethyl)pyridin-2-yl)piperazin-1-yl)methyl)pyridin-2-yl) urea (Compound 273)

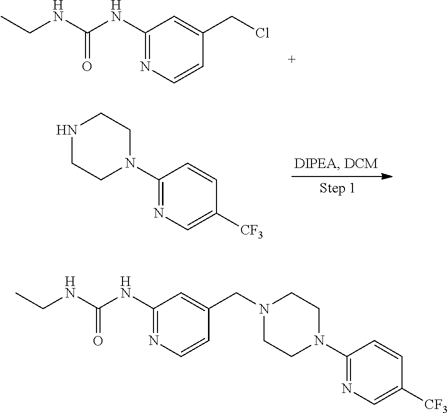 [SMILES: CCNC(=O)Nc1cc(CCl)ccn1 ; FC(F)(F)c1ccc(N2CCNCC2)nc1 ; CCNC(=O)Nc1cc(CN2CCN(c3ccc(C(F)(F)F)cn3)CC2)ccn1]

Step 1: Synthesis of 1-ethyl-3-(4-((4-(5-(trifluoromethyl)pyridin-2-yl)piperazin-1-yl)methyl)pyridin-2-yl)urea: To a stirred solution of 1-(5-(trifluoromethyl)pyridin-2-yl) piperazine (100 mg, 0.43 mmol) in DCM (4 mL) were added 1-(4-(chloromethyl) pyridin-2-yl)-3-ethylurea (92 mg, 0.43 mmol) and DIPEA (168 mg, 1.3 mmol) and the reaction was stirred at 40° C. for 2 h. The reaction mixture was quenched with water and extracted with DCM:MeOH (10:1, 3 mL×3). The combined organic layers were washed with brine, dried over Na₂SO₄, filtered, and the filtrate was concentrated under reduced pressure. The residue was purified by silica gel chromatography to give the crude product which was purified by prep-HPLC to afford the title compound (115 mg, 65%). LCMS (m/z): 409.3 [M+H]⁺; ¹H NMR (400 MHz, CD₃OD) δ 8.35 (s, 1H), 8.16 (d, J=5.3 Hz, 1H), 7.73 (dd, J=9.1, 2.3 Hz, 1H), 7.13 (s, 1H), 7.01 (d, J=5.2 Hz, 1H), 6.89 (d, J=9.1 Hz, 1H), 3.77-3.65 (m, 4H), 3.57 (s, 2H), 3.39-3.34 (m, 2H), 2.64-2.52 (m, 4H), 1.23 (t, J=7.2 Hz, 3H).

### Example S-40: Synthesis of 1-(4-((4-(6-cyanopyridin-3-yl) piperazin-1-yl) methyl) pyridin-2-yl)-3-ethylurea formate (Compound 267)

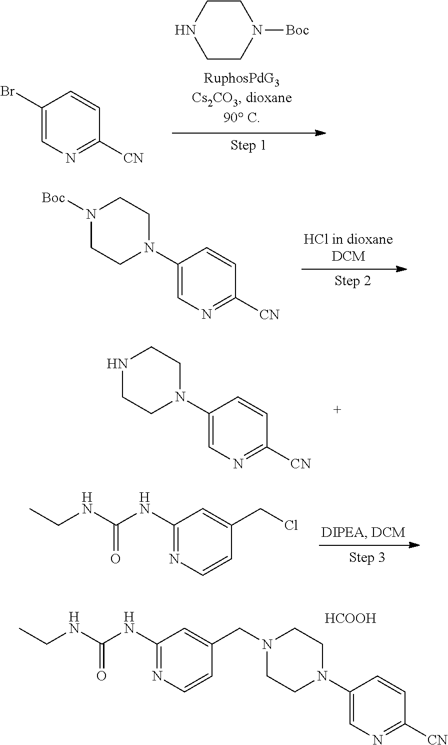

Step 1: Synthesis of tert-butyl 4-(6-cyanopyridin-3-yl) piperazine-1-carboxylate: To a solution of 5-bromopicolinonitrile (400 mg, 2.19 mmol) in dioxane (10 mL) were added tert-butyl piperazine-1-carboxylate (611 mg, 3.28 mmol), RuPhosPdG3 (184 mg, 0.22 mmol), Cs₂CO₃ (1.42 g, 4.37 mmol) and the reaction mixture was stirred at 90° C. for 18 h under N2 atmosphere. The reaction mixture was quenched with saturated NH₄Cl and extracted with EtOAc (20 mL×3). The combined organic layers were washed with brine, dried over Na₂SO₄, filtered, and the filtrate was concentrated under reduced pressure. The residue was purified by silica gel chromatography to afford the title compound (300 mg, 48%). LCMS (m/z): 289.2 [M+H]⁺.

Step 2: Synthesis of 5-(piperazin-1-yl) picolinonitrile: To a solution of tert-butyl 4-(6-cyanopyridin-3-yl) piperazine-1-carboxylate (280 mg, 0.97 mmol) in DCM (4 mL) was added 1M HCl in dioxane (1 mL) and the reaction was stirred at 25° C. for 5 h. The reaction mixture was quenched with saturated NaHCO₃ to pH 8 and extracted with DCM:MeOH (10:1, 10 mL×3). The combined organic layers were washed with brine, dried over Na₂SO₄, filtered, and the filtrate was concentrated under reduced pressure. The residue was purified by silica gel chromatography to afford the title compound (180 mg, 98%). LCMS (m/z): 189.2 [M+H]⁺.

Step 3: Synthesis of 1-(4-((4-(6-cyanopyridin-3-yl)piperazin-1-yl)methyl)pyridin-2-yl)-3-ethylurea formate: To a solution of 5-(piperazin-1-yl) picolinonitrile (180 mg, 0.96 mmol) in DCM (5 mL) were added 1-(4-(chloromethyl) pyridin-2-yl)-3-ethylurea (205 mg, 0.96 mmol) and DIPEA (371 mg, 2.87 mmol) and the reaction was stirred at 40° C. for 2 h. The reaction mixture was quenched with water and extracted with DCM:MeOH (10:1, 10 mL×3). The combined organic layers were washed with brine, dried over Na₂SO₄, filtered, and the filtrate was concentrated under reduced pressure. The residue was purified by silica gel chromatography to give the crude product which was purified by prep-HPLC to afford the title compound (98 mg, 25%). LCMS (m/z): 366.3 [M+H]⁺; ¹H NMR (400 MHz, CD₃OD) δ 8.36 (d, J=2.9 Hz, 1H), 8.17 (d, J=5.3 Hz, 1H), 8.12 (s, 1H), 7.66 (d, J=8.9 Hz, 1H), 7.37 (dd, J=8.9, 3.0 Hz, 1H), 7.16 (s, 1H), 7.02 (d, J=5.3 Hz, 1H), 3.62 (s, 2H), 3.55-3.44 (m, 4H), 3.38-3.34 (m, 2H), 2.73-2.61 (m, 4H), 1.22 (t, J=7.2 Hz, 3H).

### Example S-41: Synthesis of 5-(4-((2-(3-ethylureido) pyridin-4-yl) methyl)-3-oxopiperazin-1-yl)-N,6-dimethylpicolinamide (Compound 29)

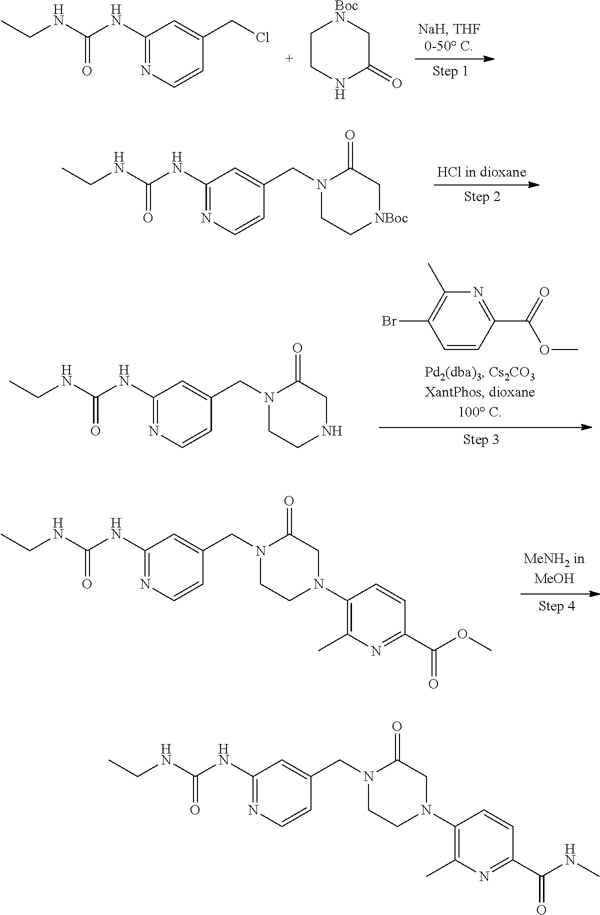

Step 1: Synthesis of tert-butyl 4-((2-(3-ethylureido)pyridin-4-yl)methyl)-3-oxopiperazine-1-carboxylate: To a solution of tert-butyl 3-oxopiperazine-1-carboxylate (1.13 g, 5.6 mmol) in dry THF (10 mL) was added NaH (60%, 224 mg, 5.6 mmol) and the reaction mixture was stirred at 0° C. for 30 min. Then 1-(4-(chloromethyl)pyridin-2-yl)-3-ethylurea (1 g, 4.7 mmol) was added at 0° C. and the resulting solution was stirred at 50° C. for 16 h. The reaction was quenched with water (50 mL) and extracted with EtOAc (50 mL×3). The combined organic layers were washed with brine, dried over Na₂SO₄, filtered, and the filtrate was concentrated under reduced pressure. The residue was purified by silica gel chromatography to afford the title compound (800 mg, 45%). LCMS (m/z): 378.1 [M+H]⁺.

Step 2: Synthesis of 1-ethyl-3-(4-((2-oxopiperazin-1-yl)methyl)pyridin-2-yl)urea: To a solution of tert-butyl 4-((2-(3-ethylureido)pyridin-4-yl)methyl)-3-oxopiperazine-1-carboxylate (800 mg, 2.12 mmol) in DCM (4 mL) was added 1M HCl in dioxane (4 mL) and the resulting solution was stirred at RT for 5 h. The reaction mixture was quenched with saturated NaHCO₃ to pH 8 and extracted with DCM:MeOH (10:1, 10 mL×3). The combined organic layers were washed with brine, dried over Na₂SO₄, filtered, and the filtrate was concentrated under reduced pressure. The residue was purified by silica gel chromatography to afford the title compound (550 mg, 94%). LCMS (m/z): 278.1 [M+H]⁺.

Step 3: Synthesis of methyl 5-(4-((2-(3-ethylureido)pyridin-4-yl)methyl)-3-oxopiperazin-1-yl)-6-methylpicolinate: To a stirred solution of methyl 5-bromo-6-methylpyridine-2-carboxylate (100 mg, 0.43 mmol) in 1,4-dioxane (5 mL) were added 1-ethyl-3-(4-((2-oxopiperazin-1-yl)methyl)pyridin-2-yl)urea (133 mg, 0.48 mmol), Pd₂(dba)₃ (82 mg, 0.09 mmol), XantPhos (23 mg, 0.04 mmol), and Cs₂CO₃ (283 mg, 0.87 mmol) and the reaction mixture was stirred at 100° C. for 16 h under N2 atmosphere. The reaction was quenched with water (20 mL) and extracted with EtOAc (20 mL×3). The combined organic layers were washed with brine, dried over Na₂SO₄, filtered, and the filtrate was concentrated under reduced pressure. The residue was purified by silica gel chromatography to afford the title compound (50 mg, 27%). LCMS (m/z): 427.3 [M+H]⁺.

Step 4: Synthesis of 5-(4-((2-(3-ethylureido)pyridin-4-yl)methyl)-3-oxopiperazin-1-yl)-N,6-dimethylpicolinamide: To a stirred solution of methyl 5-(4-((2-(3-ethylureido)pyridin-4-yl)methyl)-3-oxopiperazin-1-yl)-6-methylpicolinate (45 mg, 0.11 mmol) in MeOH (1 mL) was added 33% MeNH₂ in MeOH (1 mL) and the reaction was stirred at 25° C. for 18 h. The reaction mixture was concentrated under reduced pressure and the residue was purified by prep-HPLC to afford the title compound (9.5 mg, 21%). LCMS (m/z): 426.3 [M+H]; ¹H NMR (400 MHz, CD₃OD) δ 8.15 (d, J=5.2 Hz, 1H), 7.89 (d, J=8.3 Hz, 1H), 7.53 (d, J=8.3 Hz, 1H), 7.02 (s, 1H), 6.87 (d, J=5.0 Hz, 1H), 4.64 (s, 2H), 3.80 (s, 2H), 3.48 (t, J=5.0 Hz, 2H), 3.38-3.30 (m, 4H), 2.92 (s, 3H), 2.57 (s, 3H), 1.18 (t, J=7.2 Hz, 3H).

### Example S-42: Synthesis of 5-(4-((2-(3-ethylureido) pyridin-4-yl) methyl) piperazin-1-yl)-N-methyl-6-(methyl-d₃) picolinamide hemiformate (Compound 274)

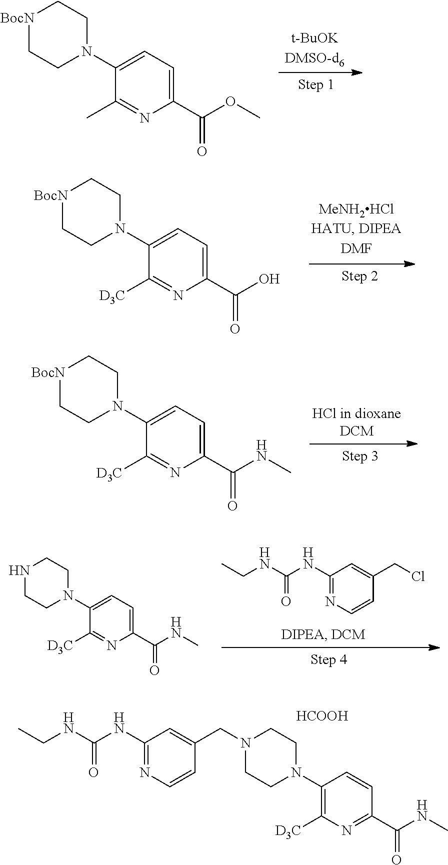

Step 1: Synthesis of 5-(4-(tert-butoxy carbonyl) piperazin-1-yl)-6-(methyl-d₃) picolinic acid: To a stirred solution of tert-butyl 4-(6-(methoxycarbonyl)-2-methylpyridin-3-yl)piperazine-1-carboxylate (200 mg, 0.6 mmol) in DMSO-d₆ (3 mL) was added t-BuOK (202 mg, 1.8 mmol) and the reaction was stirred at 25° C. for 18 h. The reaction mixture was quenched with aqueous NH₄C₁ to pH 7 and extracted with EtOAc (10 mL×3). The aqueous layer was concentrated under reduced pressure and the residue was purified by C₁₈ chromatography to afford the title compound (150 mg, 77%). LCMS (m/z): 325.3 [M+H]⁺.

Step 2: Synthesis of tert-butyl 4-(2-(methyl-d₃)-6-(methyl carbamoyl) pyridin-3-yl) piperazine-1-carboxylate: To a stirred solution of 5-(4-(tert-butoxy carbonyl)piperazin-1-yl)-6-(methyl-d₃) picolinic acid (130 mg, 0.4 mmol) in DMF (3 mL) were added MeNH₂HCl (40 mg, 0.6 mmol), HATU (228 mg, 0.6 mmol), and DIPEA (155 mg, 1.2 mmol), and the reaction was stirred at 25° C. for 18 h. The reaction mixture was quenched with water and extracted with EtOAc (10 mL×2). The combined organic layers were washed with brine, dried over Na₂SO₄, filtered, and the filtrate was concentrated under reduced pressure. The residue was purified by silica gel chromatography to afford the title compound (130 mg, 96%). LCMS (m/z): 338.2 [M+H]⁺.

Step 3: Synthesis of N-methyl-6-(methyl-d₃)-5-(piperazin-1-yl) picolinamide: To a stirred solution of tert-butyl 4-(2-(methyl-d₃)-6-(methyl carbamoyl) pyridin-3-yl) piperazine-1-carboxylate (120 mg, 0.36 mmol) in DCM (4 mL) was added 1M HCl in dioxane (1 mL) and the reaction was stirred at 25° C. for 6 h. The reaction mixture was quenched with aq. NaHCO₃ to pH 7 and extracted with DCM:MeOH (10:1, 5 mL×3). The combined organic layers were washed with brine, dried over Na₂SO₄, filtered, and the filtrate was concentrated under reduced pressure. The residue was purified by silica gel chromatography to afford the title compound (80 mg, 94%). LCMS (m/z): 238.3 [M+H]⁺.

Step 4: Synthesis of 5-(4-((2-(3-ethylureido) pyridin-4-yl) methyl) piperazin-1-yl)-N-methyl-6-(methyl-d₃) picolinamide hemiformate: To a stirred solution of N-methyl-6-(methyl-d₃)-5-(piperazin-1-yl) picolinamide (80 mg, 0.34 mmol) in DCM (4 mL) were added 1-(4-(chloromethyl)pyridin-2-yl)-3-ethylurea (72 mg, 0.34 mmol), and DIPEA (131 mg, 1.01 mmol) and the reaction was stirred at 25° C. for 18 h. The reaction mixture was quenched with water and extracted with DCM (5 mL×3). The combined organic layers were washed with brine, dried over Na₂SO₄, filtered, and the filtrate was concentrated under reduced pressure. The residue was purified by prep-HPLC to afford the title compound (25 mg, 17%). LCMS (m/z): 415.3 [M+H]⁺, ¹H NMR (400 MHz, CD-OD) δ 8.21 (d, J=5.3 Hz, 1H), 8.14 (s, 0.5H), 7.89 (d, J=8.3 Hz, 1H), 7.52 (d, J=8.3 Hz, 1H), 7.22 (s, 1H), 7.05 (d, J=5.0 Hz, 1H), 3.78 (s, 2H), 3.37 (q, J=7.2 Hz, 2H), 3.14-3.07 (m, 4H), 2.96 (s, 3H), 2.91-2.80 (m, 4H), 1.23 (t, J=7.2 Hz, 3H).

### Example S-43: Synthesis of 1-(4-((4-(5-cyanopyridin-2-yl) piperazin-1-yl) methyl)-3-fluoropyridin-2-yl)-3-ethylurea (Compound 268)

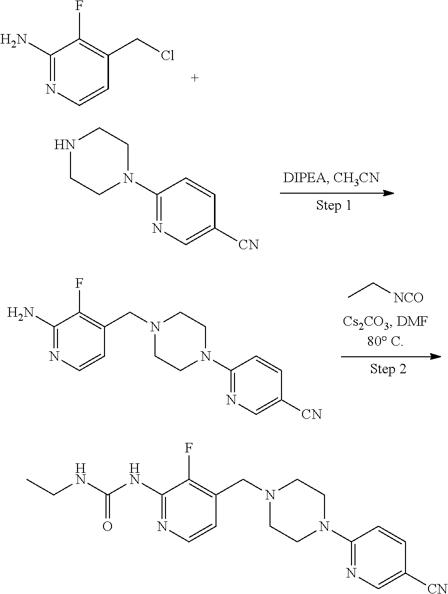

Step 1: Synthesis of 6-(4-((2-amino-3-fluoropyridin-4-yl) methyl) piperazin-1-yl) nicotinonitrile: To a stirred solution of 4-(chloromethyl)-3-fluoropyridin-2-amine (200 mg, 1.25 mmol) in CH₃CN (4 mL) were added 6-(piperazin-1-yl)nicotinonitrile (235 mg, 1.25 mmol) and DIPEA (483 mg, 3.74 mmol) and the reaction mixture was stirred at 25° C. for 18 h. The reaction mixture was quenched with water and extracted with DCM (15 mL/3). The combined organic layers were washed with brine, dried over Na₂SO₄, filtered, and the filtrate was concentrated under reduced pressure. The residue was purified by silica gel chromatography to afford the title compound (150 mg, 38%). LCMS (m/z): 313.2 [M+H]⁺.

Step 2: Synthesis of 1-(4-((4-(5-cyanopyridin-2-yl) piperazin-1-yl) methyl)-3-fluoro pyridin-2-yl)-3-ethylurea: To a stirred solution of 6-(4-((2-amino-3-fluoropyridin-4-yl)methyl) piperazin-1-yl)nicotinonitrile (140 mg, 0.45 mmol) in DMF (4 mL) were added ethyl isocyanate (48 mg, 0.67 mmol), Cs₂CO₃ (292 mg, 0.9 mmol) and the reaction mixture was stirred at 80° C. for 2 h. The reaction mixture was quenched with aq. NH₄Cl to pH 7 and extracted with DCM:MeOH (10:1, 5 mL×3). The combined organic layers were washed with brine, dried over Na₂SO₄, filtered, and the filtrate was concentrated under reduced pressure. The residue was purified by prep-HPLC to afford the title compound (43 mg, 25%). LCMS (m/z): 384.3 [M+H]⁺; ¹H NMR (400 MHz, CD₃OD) δ 8.36 (d, J=2.0 Hz, 1H), 7.99 (d, J=5.2 Hz, 1H), 7.69 (dd, J=9.1, 2.3 Hz, 1H), 7.12 (t, J=5.0 Hz, 1H), 6.81 (d, J=9.1 Hz, 1H), 3.74-3.67 (m, 4H), 3.65 (s, 2H), 3.34 (q, J=7.2 Hz, 2H), 2.60-2.49 (m, 4H), 1.19 (t, J=7.2 Hz, 3H).

### Example S-44: Synthesis of 1-(6-((4-(5-cyanopyridin-2-yl) piperazin-1-yl) methyl) pyrimidin-4-yl)-3-ethylurea (Compound 275)

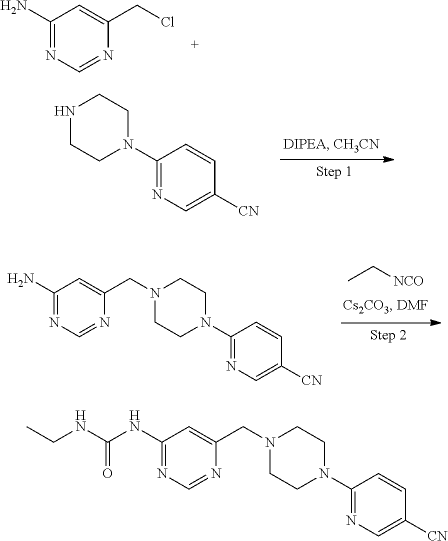

Step 1: Synthesis of 6-(4-((6-aminopyrimidin-4-yl) methyl) piperazin-1-yl) nicotinonitrile: To a stirred solution of 6-(chloromethyl) pyrimidin-4-amine (140 mg, 0.98 mmol) in CH₃CN (3 mL) were added 6-(piperazin-1-yl) nicotinonitrile (184 mg, 0.98 mmol), DIPEA (378 mg, 2.93 mmol) and the reaction mixture was stirred at 25° C. for 18 h. The reaction mixture was quenched with water and extracted with DCM (10 mL×3). The combined organic layers were washed with brine, dried over Na₂SO₄, filtered, and the filtrate was concentrated under reduced pressure. The residue was purified by silica gel chromatography to afford the title compound (110 mg, 38%). LCMS (m/z): 296.2 [M+H]⁺.

Step 2: Synthesis of 1-(6-((4-(5-cyanopyridin-2-yl) piperazin-1-yl) methyl) pyrimidin-4-yl)-3-ethylurea: To a stirred solution of 6-(4-((6-aminopyrimidin-4-yl) methyl) piperazin-1-yl) nicotinonitrile (100 mg, 0.34 mmol) in DMF (2 mL) at 25° C. were added ethyl isocyanate (36 mg, 0.51 mmol), Cs₂CO₃ (222 mg, 0.68 mmol) and the reaction mixture stirred at 80° C. for 2 h. The reaction mixture was quenched with aqueous NH₄Cl to pH 7 and extracted with DCM:MeOH (10:1, 5 mL×3). The combined organic layers were washed with brine, dried over Na₂SO₄, filtered, and the filtrate was concentrated under reduced pressure. The residue was purified by prep-HPLC to afford the title compound (71 mg, 57%). LCMS (m/z): 367.3[M+H]⁺; ¹H NMR (400 MHz, CD₃OD) δ 8.64 (s, 1H), 8.37 (d, J=1.9 Hz, 1H), 7.69 (dd, J=9.1, 2.2 Hz, 1H), 7.42 (s, 1H), 6.83 (d, J=9.1 Hz, 1H), 3.77-3.70 (m, 4H), 3.59 (s, 2H), 3.32 (q, J=7.2 Hz, 2H), 2.63-2.56 (m, 4H), 1.18 (t, J=7.2 Hz, 3H).

### Example S-45: Synthesis of 1-(4-((4-(4-cyano-2-fluorophenyl)piperazin-1-yl)methyl)pyridin-2-yl)-3-ethylurea trifluoroacetate (Compound 276)

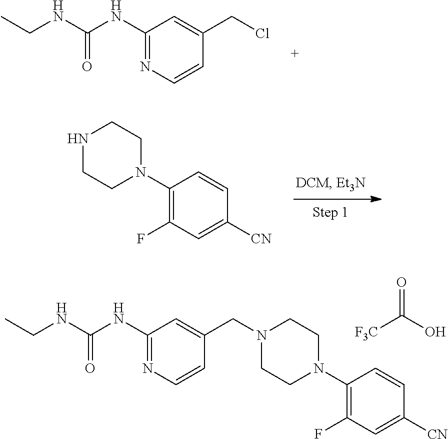

Step 1: Synthesis of 1-(4-((4-(4-cyano-2-fluorophenyl)piperazin-1-yl)methyl)pyridin-2-yl)-3-ethylurea trifluoroacetate: A solution of 1-(4-(chloromethyl)pyridin-2-yl)-3-ethylurea (50 mg, 0.23 mmol), 3-fluoro-4-(piperazin-1-yl)benzonitrile (53 mg, 0.26 mmol), and Et-N (47.4 mg, 0.47 mmol) in DCM (3 mL) was stirred at 20° C. for 3 h. The reaction mixture was quenched with water and extracted with EtOAc (20 mL×3). The combined organic layers were washed with brine, dried over Na₂SO₄, filtered, and the filtrate was concentrated under reduced pressure. The residue was purified by Prep-HPLC to afford the title compound (32 mg, 23%). LCMS (m/z): 382.9 [M+H]⁺; ¹H NMR (400 MHz, CD₃OD) δ 8.33 (d, J=5.6 Hz, 1H), 7.54 (d, J=2.0 Hz, 1H), 7.52 (s, 1H), 7.48 (s, 1H), 7.25-7.17 (m, 2H), 4.36 (s, 2H), 3.46-3.57 (m, 4H), 3.39-3.44 (m, 4H), 3.33-3.38 (m, 2H), 1.22 (t, J=7.2 Hz, 3H).

### Example S-46: Synthesis of 5-(4-((2-(3-ethylureido) oxazol-5-yl) methyl) piperazin-1-yl)-N,6-dimethylpicolinamide (Compound 246)

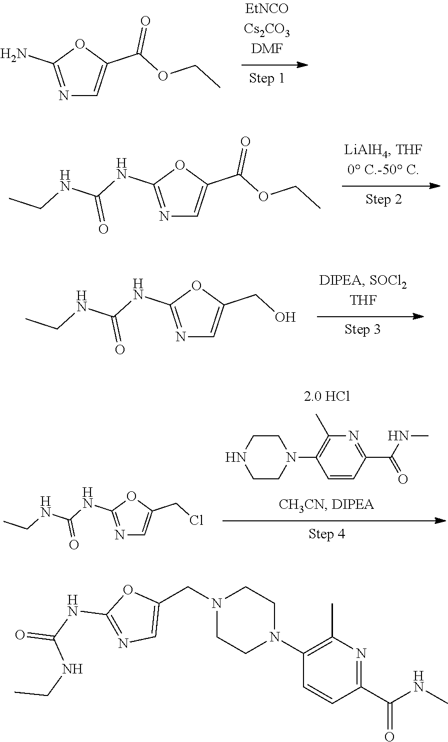

Step 1: Synthesis of ethyl 2-(3-ethylureido) oxazole-5-carboxylate: To a stirred solution of ethyl 2-aminooxazole-5-carboxylate (200 mg, 1.28 mmol) in DMF (6 mL) was added ethyl isocyanate (109 mg, 1.54 mmol), Cs₂CO₃ (834 mg, 2.56 mmol) and the reaction mixture was stirred at 25° C. for 18 h. The reaction mixture was quenched with aqueous NH₄Cl to pH 7 and extracted with EtOAc (20 mL×3). The combined organic layers were washed with brine, dried over Na₂SO₄, filtered, and the filtrate was concentrated under reduced pressure. The residue was purified by silica gel chromatography to afford the title compound (150 mg, 52%). LCMS: 228.2 [M+H]⁺.

Step 2: Synthesis of 1-ethyl-3-(5-(hydroxymethyl) oxazol-2-yl) urea: To a stirred solution of ethyl 2-(3-ethylureido) oxazole-5-carboxylate (140 mg, 0.62 mmol) in THF (3 mL) at 0° C. was added LiAlH₄ (0.9 mL, 1M in THF) and the reaction mixture was stirred at 50° C. for 1 h. The reaction mixture was quenched with Na₂SO₄·10 H₂O. The reaction mixture was filtered, and the filter cake was washed with DCM/MeOH (10:1, 5 mL×3). The filtrate was concentrated under reduced pressure and the residue was purified by silica gel chromatography to afford the title compound (110 mg, 96%). LCMS (m/z): 186.1 [M+H]⁺

Step 3: Synthesis of 1-(5-(chloromethyl)oxazol-2-yl)-3-ethylurea: To a stirred solution of 1-ethyl-3-(5-(hydroxymethyl) oxazol-2-yl) urea (80 mg, 0.43 mmol) in THF (2 mL) at 0° C. were added DIPEA (61 mg, 0.47 mmol), SOCl₂ (51 mg, 0.43 mmol) and the reaction mixture was stirred at 25° C. for 2 h. The reaction mixture was concentrated under reduced pressure to afford the title compound as crude (100 mg) which was directly used in the next reaction without further purification.

Step 4: Synthesis of 5-(4-((2-(3-ethylureido)oxazol-5-yl)methyl)piperazin-1-yl)-N,6-dimethylpicolinamide: To a stirred solution of 1-(5-(chloromethyl) oxazol-2-yl)-3-ethylurea (100 mg, crude) in CH₃CN (3 mL) were added N,6-dimethyl-5-(piperazin-1-yl)picolinamide dihydrochloride (151 mg, 0.49 mmol), DIPEA (190 mg, 1.47 mmol) and the reaction mixture was stirred at 25° C. for 18 h. The reaction mixture was quenched with water and extracted with DCM/MeOH (10:1, 5 mL×3). The combined organic layers were washed with brine, dried over Na₂SO₄, filtered, and the filtrate was concentrated under reduced pressure. The residue was purified by silica gel chromatography to give the crude product, which was further purified by prep-HPLC to afford the title compound (12 mg, 7%). LCMS (m/z): 402.3 [M+H]⁺; ¹H NMR (400 MHz, CD₃OD) δ 7.84 (d, J=8.3 Hz, 1H), 7.46 (d, J=8.3 Hz, 1H), 6.87 (s, 1H), 3.66 (s, 2H), 3.32 -3.29 (m, 2H), 3.07-2.97 (m, 4H), 2.91 (s, 3H), 2.79-2.67 (m, 4H), 2.51 (s, 3H), 1.17 (t, J=7.2 Hz, 3H).

### Example S-47: Synthesis of 5-(4-((2-(3-ethylureido)thiazol-5-yl)methyl)piperazin-1-yl)-N,6-dimethylpicolinamide trifluoroacetate (Compound 242)

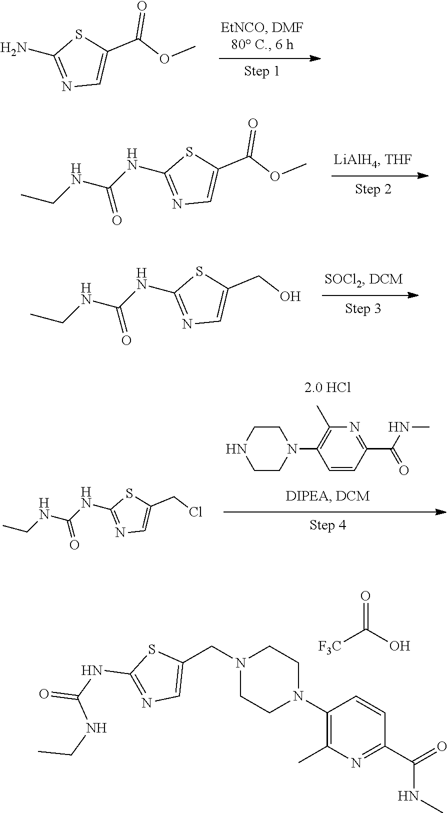

Step 1: Synthesis of methyl 2-(3-ethylureido)thiazole-5-carboxylate: To a solution of methyl 2-aminothiazole-5-carboxylate (1.1 g, 6.95 mmol) in DMF (13 mL) was added ethyl isocyanate (989 mg, 13.91 mmol) and the reaction mixture was stirred at 80° C. for 6 h. The reaction was quenched with water and extracted with EtOAc (100 mL×3). The combined organic layers were washed with brine, dried over Na₂SO₄, filtered and the filtrate was concentrated under reduced pressure. The residue was purified by silica gel chromatography to afford the title compound (1.4 g, 88%). LCMS (m/z): 230.0 [M+H]⁺.

Step 2: Synthesis of 1-ethyl-3-(5-(hydroxymethyl)thiazol-2-yl)urea: To a solution of methyl 2-(3-ethylureido)thiazole-5-carboxylate (300 mg, 1.31 mmol), HMPA (470 mg, 2.62 mmol) in THF (4 mL) was added 1M solution of LiAlH₄ in THF (2.6 mL, 2.62 mmol) dropwise and the reaction mixture was stirred at 20° C. for 1 h. The reaction was quenched with sodium sulfate decahydrate, filtered, and the filtrate was concentrated under reduced pressure. The residue was purified by silica gel chromatography to afford the title compound (110 mg, 42%). LCMS (m/z): 202.1 [M+H]⁺.

Step 3: Synthesis of 1-(5-(chloromethyl)thiazol-2-yl)-3-ethylurea: To a solution of 1-ethyl-3-(5-(hydroxymethyl)thiazol-2-yl)urea (60 mg, 0.30 mmol) in DCM (2 mL) was added SOCl₂ (71 mg, 0.60 mmol) dropwise and the reaction mixture was stirred at RT for 1 h. The reaction was concentrated under reduced pressure to afford the title compound (40 mg, crude). LCMS (m/z): 216.1 [M−Cl+OCH₃]⁺.

Step 4: Synthesis of 5-(4-((2-(3-ethylureido)thiazol-5-yl)methyl)piperazin-1-yl)-N,6-dimethylpicolinamide trifluoroacetate: A solution of 1-(5-(chloromethyl)thiazol-2-yl)-3-ethylurea (40 mg, crude), N,6-dimethyl-5-(piperazin-1-yl)picolinamide dihydrochloride (46 mg, 0.15 mmol) and DIPEA (97 mg, 0.75 mmol) in DCM (2 mL) was stirred at 20° C. for 2 h. The reaction mixture was quenched with water and extracted with EtOAc (30 mL×3). The combined organic layers were washed with brine, dried over Na₂SO₄, filtered and the filtrate was concentrated under reduced pressure. The residue was purified by prep-HPLC to afford the title compound (9 mg, 11%). LCMS (m/z): 418.1 [M+H]; ¹H NMR (400 MHz, CD₃OD) δ 7.93 (d, J=8.3 Hz, 1H), 7.59 (d, J=8.3 Hz, 1H), 7.55 (s, 1H), 4.66 (s, 2H), 3.67-3.61 (m, 2H), 3.55-3.38 (m, 4H), 3.31-3.24 (m, 2H), 3.18-3.11 (m, 2H), 2.96 (s, 3H), 2.61 (s, 3H), 1.19 (t, J=7.2 Hz, 3H).

### Example S-48: Synthesis of 5-(4-((2-(3-ethylureido)-1-methyl-1H-imidazol-5-yl) methyl) piperazin-1-yl)-N,6-dimethylpicolinamide (Compound 277)

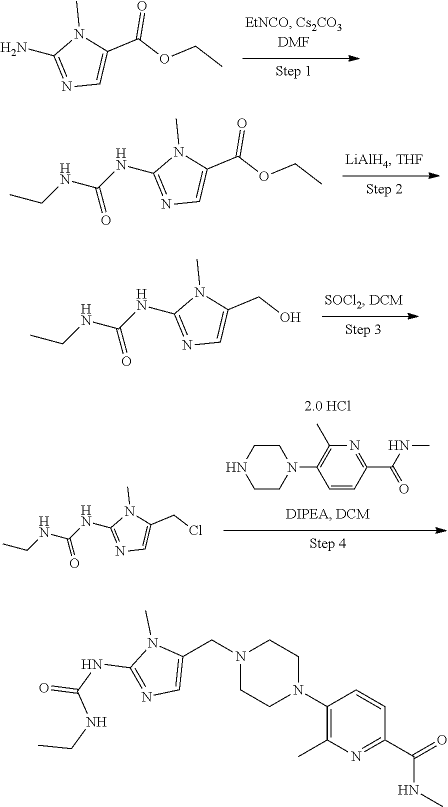

Step 1: Synthesis of ethyl 2-(3-ethylureido)-1-methyl-1H-imidazole-5-carboxylate: To a stirred solution of ethyl 2-amino-1-methyl-1H-imidazole-5-carboxylate (200 mg, 1.18 mmol) in DMF (4 mL) was added ethyl isocyanate (101 mg, 1.42 mmol), Cs₂CO₃ (769 mg, 2.36 mmol) and the reaction mixture was stirred at 25° C. for 18 h. The reaction mixture was quenched with aqueous NH₄Cl to pH=7 and extracted with EtOAc (20 mL×3). The combined organic layers were washed with brine, dried over Na₂SO₄, filtered and the filtrate was concentrated under reduced pressure. The residue was purified by silica gel chromatography to afford the title compound (100 mg, 35%). LCMS (m/z): 241.2 [M+H]⁺.

Step 2: Synthesis of 1-ethyl-3-(5-(hydroxymethyl)-1-methyl-1H-imidazol-2-yl) urea: To a stirred solution of ethyl 2-(3-ethylureido)-1-methyl-1H-imidazole-5-carboxylate (90 mg, 0.37 mmol) in THF (1 mL) at 0° C. was added LiAlH₄ (0.6 mL, 0.6 mmol, 1M in THF) and the reaction mixture was stirred at 50° C. for 1 h. The reaction mixture was quenched with sodium sulfate decahydrate, filtered and the filter cake was washed with MeOH (5 mL×3). The filtrate was concentrated under reduced pressure and the residue was purified by silica gel chromatography to afford the title compound (60 mg, 82%). LCMS (m/z): 199.2 [M+H]⁺.

Step 3: Synthesis of 1-(5-(chloromethyl)-1-methyl-1H-imidazol-2-yl)-3-ethylurea: To a stirred solution of 1-ethyl-3-(5-(hydroxymethyl)-1-methyl-1H-imidazol-2-yl) urea (20 mg, 0.10 mmol) in DCM (2 mL) at 0° C. was added SOCl₂ (14 mg, 0.12 mmol) and the reaction mixture was stirred at 25° C. for 5 h. The reaction mixture was concentrated under reduced pressure to afford the title compound (22 mg, crude).

Step 4: Synthesis of 5-(4-((2-(3-ethylureido)-1-methyl-1H-imidazol-5-yl) methyl) piperazin-1-yl)-N,6-dimethylpicolinamide: To a stirred solution of 1-(5-(chloromethyl)-1-methyl-1H-imidazol-2-yl)-3-ethylurea (22 mg, crude) in CH₃CN (1 mL) was added N,6-dimethyl-5-(piperazin-1-yl)picolinamide dihydrochloride (31 mg, 0.10 mmol), DIPEA (65 mg, 0.50 mmol) and the reaction mixture was stirred at 25° C. for 18 h. The reaction mixture was quenched with water and extracted with EtOAc (5 mL×3). The combined organic layers were washed with brine, dried over Na₂SO₄, filtered and the filtrate was concentrated under reduced pressure. The residue was purified by silica gel chromatography to furnish the crude product which was purified by prep-HPLC to afford the title compound (2 mg, 5%). LCMS (m/z): 415.2 [M+H]⁺; ¹H NMR (400 MHz, CD₃OD) δ 7.86 (d, J=8.3 Hz, 1H), 7.47 (d, J=8.3 Hz, 1H), 6.91 (s, 1H), 3.62 (s, 5H), 3.29-3.24 (m, 2H), 3.07-2.97 (m, 4H), 2.94 (s, 3H), 2.75-2.66 (m, 4H), 2.55 (s, 3H), 1.17 (t, J=7.2 Hz, 3H).

### Example S-49: Synthesis of 5-(4-((6-(3-ethylureido)-5-fluoropyrimidin-4-yl) methyl) piperazin-1-yl)-6-fluoro-N-methylpicolinamide (Compound 278)

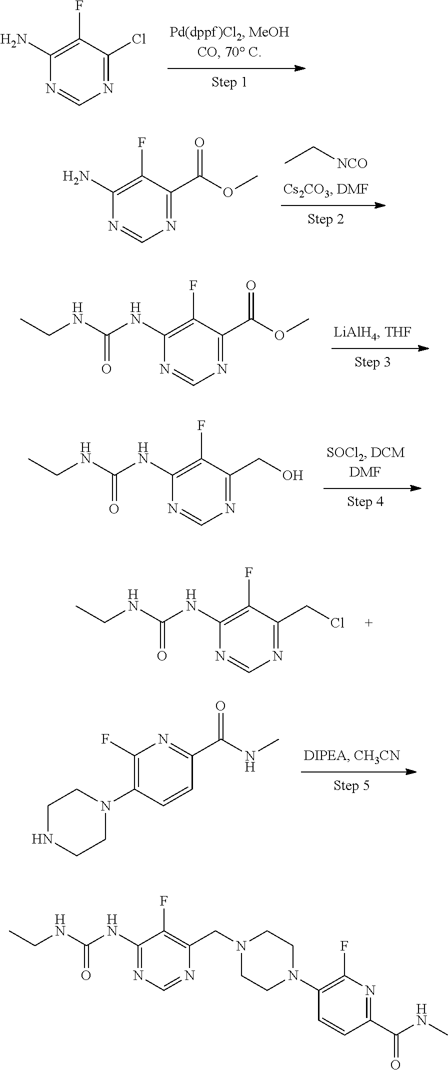

Step 1: Synthesis of methyl 6-amino-5-fluoropyrimidine-4-carboxylate: To a stirred solution of 6-chloro-5-fluoropyrimidin-4-amine (1 g, 6.78 mmol) in MeOH (20 mL) was added Et₃N (2.06 g, 20.37 mmol), Pd(dppf)C₁₋₂ (0.5 g, 0.68 mmol) and the reaction mixture was stirred at 70° C. for 18 h under CO atmosphere. The reaction mixture was quenched with water and extracted with EtOAc (20 mL×3). The combined organic layers were washed with brine, dried over Na₂SO₄, filtered and the filtrate was concentrated under reduced pressure. The residue was purified by silica gel chromatography to afford the title compound (500 mg, 43%). LCMS (m/z): 172.1 [M+H]⁺.

Step 2: Synthesis of methyl 6-(3-ethylureido)-5-fluoropyrimidine-4-carboxylate: To a stirred solution of methyl 6-amino-5-fluoropyrimidine-4-carboxylate (490 mg, 2.86 mmol) in DMF (10 mL) were added ethyl isocyanate (244 mg, 3.43 mmol), Cs₂CO₃ (1.87 g, 5.73 mmol) and the reaction mixture was stirred at 25° C. for 18 h. The reaction mixture was quenched with aqueous NH₄Cl to pH=7, and extracted with EtOAc (10 mL×3). The combined organic layers were washed with brine, dried over Na₂SO₄, filtered and the filtrate was concentrated under reduced pressure. The residue was purified by silica gel chromatography to afford the title compound (350 mg, 51%). LCMS (m/z): 243.2 [M+H]⁺.

Step 3: Synthesis of 1-ethyl-3-(5-fluoro-6-(hydroxymethyl) pyrimidin-4-yl) urea: To a stirred solution of methyl 6-(3-ethylureido)-5-fluoropyrimidine-4-carboxylate (340 mg, 1.40 mmol) in THF (5 mL) at 0° C. was added 1M LiAlH₄ in THF (2.1 mL, 2.1 mmol) and the reaction mixture was stirred at 25° C. for 2 h. The reaction mixture was quenched with sodium sulfate decahydrate, filtered and the filter cake was washed with MeOH (5 mL×3). The filtrate was concentrated under reduced pressure and the residue was purified by silica gel chromatography to afford the title compound (200 mg, 67%). LCMS (m/z): 215.0 [M+H]⁺.

Step 4: Synthesis of 1-(6-(chloromethyl)-5-fluoropyrimidin-4-yl)-3-ethylurea: To a stirred solution of 1-ethyl-3-(5-fluoro-6-(hydroxymethyl) pyrimidin-4-yl) urea (180 mg, 0.84 mmol) in DCM (4 mL) at 0° C. were added one drop of DMF, SOCl₂ (120 mg, 1.01 mmol) and the reaction was stirred at 25° C. for 5 h. The reaction mixture was concentrated under reduced pressure and the residue was purified by silica gel chromatography to afford the title compound (100 mg, 51%). LCMS (m/z): 233.0 [M+H]⁺.

Step 5: Synthesis of 5-(4-((6-(3-ethylureido)-5-fluoropyrimidin-4-yl) methyl) piperazin-1-yl)-6-fluoro-N-methylpicolinamide: To a stirred solution of 1-(6-(chloromethyl)-5-fluoropyrimidin-4-yl)-3-ethylurea (90 mg, 0.39 mmol) in CH₃CN (4 mL) was added 6-fluoro-N-methyl-5-(piperazin-1-yl) picolinamide (93 mg, 0.39 mmol), DIPEA (150 mg, 1.16 mmol) and the reaction mixture was stirred at 25° C. for 18 h. The reaction mixture was quenched with water and extracted with EtOAc (10 mL×3). The combined organic layers were washed with brine, dried over Na₂SO₄, filtered and the filtrate was concentrated under reduced pressure. The residue was purified by silica gel chromatography to give the crude product which was purified by prep-HPLC to afford the title compound (27 mg, 16%). LCMS (m/z): 435.1 [M+H]⁺; ¹H NMR (400 MHz, DMSO-d₆) δ 9.85 (s, 1H), 8.87 (t, 0.1=5.5 Hz, 1H), 8.55 (d, 0.1=0.8 Hz, 1H), 8.40 (q, J=4.4 Hz, 1H), 7.83 (dd, J=8.0, 0.8 Hz, 1H), 7.54 (dd, J=10.6, 8.2 Hz, 1H), 3.67 (d, J=1.8 Hz, 2H), 3.30-3.23 (m, 2H), 3.17-3.10 (m, 4H), 2.76 (d, J=4.8 Hz, 3H), 2.68-2.60 (m, 4H), 1.13 (t, J=7.2 Hz, 3H).

### Example S-50: Synthesis of 5-(4-((5-(3-ethylureido)isoxazol-3-yl)methyl)piperazin-1-yl)-N,6-dimethylpicolinamide (Compound 279)

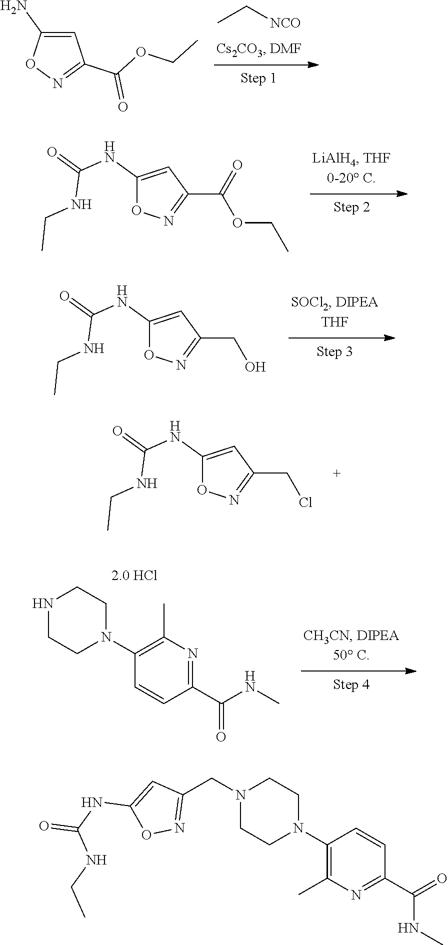

Step 1: Synthesis of ethyl 5-(3-ethylureido)isoxazole-3-carboxylate: To a solution of ethyl 5-aminoisoxazole-3-carboxylate (400 mg, 2.56 mmol) in DMF (15 mL) were added ethyl isocyanate (218 mg, 3.07 mmol), Cs₂CO₃(1.67 g, 5.12 mmol) and the reaction mixture was stirred at 20° C. for 4 h. The reaction was quenched with water and extracted with EtOAc (50 mL×3). The combined organic layers were washed with brine, dried over Na₂SO₄, filtered and the filtrate was concentrated under reduced pressure. The residue was purified by silica gel chromatography to afford the title compound (300 mg, 52%). LCMS (m/z): 228.0 [M+H]⁺.

Step 2: Synthesis of 1-ethyl-3-(3-(hydroxymethyl)isoxazol-5-yl)urea: To a solution of ethyl 5-(3-ethylureido)isoxazole-3-carboxylate (300 mg, 1.32 mmol) in THF (6 mL) at 0° C. was added a solution of 1M LiAlH₄ in THF (1.6 mL, 1.58 mmol) dropwise and the reaction mixture was stirred at 20° C. for 2 h. The reaction was quenched with Na₂SO₄·10H₂O, filtered, and the filtrate was concentrated under reduced pressure. The residue was purified by silica gel chromatography to afford the title compound (160 mg, 65%). LCMS (m/z): 186.1 [M+H]⁺.

Step 3: Synthesis of 1-(3-(chloromethyl)isoxazol-5-yl)-3-ethylurea: To a solution of 1-ethyl-3-(3-(hydroxymethyl)isoxazol-5-yl)urea (150 mg, 0.81 mmol) in THF (4 mL) at 0° C. were added SOCl₂ (145 mg, 1.22 mmol) and DIPEA (105 mg, 0.81 mmol) dropwise, and the reaction mixture was stirred at 20° C. for 1 h. The reaction was concentrated under reduced pressure and the residue was purified by silica gel chromatography to afford the title compound (90 mg, 55%). LCMS: 203.9 [M+H]⁺.

Step 4: Synthesis of 5-(4-((5-(3-ethylureido)isoxazol-3-yl)methyl)piperazin-1-yl)-N,6-dimethylpicolinamide: A solution of 1-(3-(chloromethyl)isoxazol-5-yl)-3-ethylurea (43 mg, 0.21 mmol), N,6-dimethyl-5-(piperazin-1-yl)pyridine-2-carboxamide dihydrochloride (65 mg, 0.21 mmol) and DIPEA (137 mg, 1.06 mmol) in CH₃CN (3 mL) was stirred at 50° C. for 16 h. The reaction mixture was quenched with water and extracted with EtOAc (20 mL×2). The combined organic layers were washed with brine, dried over Na₂SO₄, filtered and the filtrate was concentrated under reduced pressure. The residue was purified by prep-HPLC to afford the title compound (43 mg, 51%). LCMS (m/z): 402.2 [M+H]⁺; ¹H NMR (400 MHz, CD₃OD) δ 7.88 (d, J=8.3 Hz, 1H), 7.51 (d, J=8.3 Hz, 1H), 6.16 (s, 1H), 3.72 (s, 2H), 3.26 (q, J=7.2 Hz, 2H), 3.12-3.02 (m, 4H), 2.96 (s, 3H), 2.90-2.74 (m, 4H), 2.56 (s, 3H), 1.17 (t, J=7.2 Hz, 3H).

### Example S-51: Synthesis of 1-methyl-N2-(3-((4-(2-methyl-6-(methyl carbamoyl) pyridin-3-yl) piperazin-1-yl) methyl) isothiazol-5-yl) oxalamide formate (Compound 280)

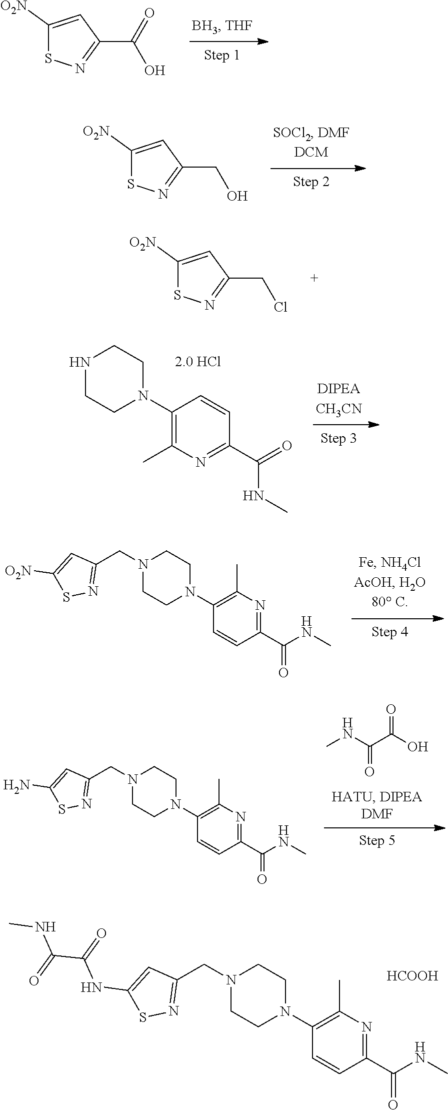

Step 1: Synthesis of (5-nitroisothiazol-3-yl) methanol: To a stirred solution of 5-nitroisothiazole-3-carboxylic acid (500 mg, 2.87 mmol) in THF (15 mL) at 0° C. was added 1M BH₃ in THF (2.9 mL) and the reaction was stirred at 25° C. for 18 h. The reaction mixture was quenched with MeOH, washed with aqueous NH₄Cl to pH 7, and extracted with EtOAc (15 mL 3). The combined organic layers were washed with brine, dried over Na₂SO₄, filtered and the filtrate was concentrated under reduced pressure. The residue was purified by silica gel chromatography to afford the title compound (410 mg, 89%). LCMS (m/z): 161.1 [M+H]⁺.

Step 2: Synthesis of 3-(chloromethyl)-5-nitroisothiazole: To a stirred solution of (5-nitroisothiazol-3-yl) methanol (410 mg, 2.56 mmol) in DCM (10 mL) were added DMF (1 drop) and SOCl₂ (305 mg, 2.56 mmol) and the reaction was stirred at 0° C. for 2 h. The reaction mixture was concentrated under reduced pressure to afford the title compound (300 mg, crude).

Step 3: Synthesis of N,6-dimethyl-5-(4-((5-nitroisothiazol-3-yl) methyl) piperazin-1-yl) picolinamide: To a stirred solution of 3-(chloromethyl)-5-nitroisothiazole (300 mg, crude) in CH₃CN (10 mL) were added methyl 6-methyl-5-(piperazin-1-yl) picolinate dihydrochloride (516 mg, 1.68 mmol), DIPEA (651 mg, 5.04 mmol) and the reaction was stirred at 25° C. for 18 h. The reaction mixture was quenched with water and extracted with EtOAc (10 mL×3). The combined organic layers were washed with brine, dried over Na₂SO₄, filtered and the filtrate was concentrated under reduced pressure. The residue was purified by silica gel chromatography to afford the title compound (280 mg, 44%). LCMS (m/z): 377.1 [M+H]⁺.

Step 4: Synthesis of 5-(4-((5-aminoisothiazol-3-yl) methyl) piperazin-1-yl)-N,6-dimethylpicolinamide: To a stirred solution of N,6-dimethyl-5-(4-((5-nitroisothiazol-3-yl)methyl)piperazin-1-yl) picolinamide (250 mg, 0.66 mmol) in AcOH (2 mL) were added Fe (185 mg, 3.31 mmol) and aqueous NH₄C₁ (0.5 mL) and the reaction was stirred at 80° C. for 3 h. The reaction mixture was quenched with aqueous NaHCO₃ to pH 8, filtered and the filter cake was washed with EtOAc (10 mL), the filtrate was extracted with EtOAc (5 mL×3). The combined organic layers were washed with brine, dried over Na₂SO₄, filtered and the filtrate was concentrated under reduced pressure. The residue was purified by silica gel chromatography to afford the title compound (140 mg, 61%). LCMS (m/z): 347.2 [M+H]⁺.

Step 5: Synthesis of N1-methyl-N2-(3-((4-(2-methyl-6-(methyl carbamoyl) pyridin-3-yl) piperazin-1-yl) methyl) isothiazol-5-yl) oxalamide formate: To a stirred solution of 5-(4-((5-aminoisothiazol-3-yl)methyl)piperazin-1-yl)-N,6-dimethylpicolinamide (50 mg, 0.14 mmol) in DMF (2 mL) were added 2-(methylamino)-2-oxoacetic acid (18 mg, 0.17 mmol), HATU (84 mg, 0.22 mmol), and DIPEA (56 mg, 0.43 mmol) and the reaction was stirred at 25° C. for 6 h. The reaction mixture was quenched with water and extracted with EtOAc (5 mL×3). The combined organic layers were washed with brine, dried over Na₂SO₄, filtered and the filtrate was concentrated under reduced pressure. The residue was purified by prep-HPLC to afford the title compound (4.3 mg, 6%). LCMS (m/z): 432.3 [M+H]⁺; ¹H NMR (400 MHz, DMSO-d₆) δ 12.71 (s, 1H), 9.06 (dd, J=9.2, 4.5 Hz, 1H), 8.38 (dd, J=9.0, 4.3 Hz, 1H), 8.10 (s, 1H), 7.75 (d, J=8.2 Hz, 1H), 7.44 (d, J=8.3 Hz, 1H), 7.19 (s, 1H), 3.59 (s, 2H), 2.93-2.88 (m, 4H), 2.76 (d, J=4.8 Hz, 3H), 2.72 (d, J=4.8 Hz, 3H), 2.55 (s, 4H), 2.45 (s, 3H).

### Example S-52: Synthesis of 5-(4-((3-(3-ethylureido)-1-methyl-1H-pyrazol-5-yl)methyl)piperazin-1-yl)-N,6-dimethylpicolinamide trihydrochloride (Compound 281)

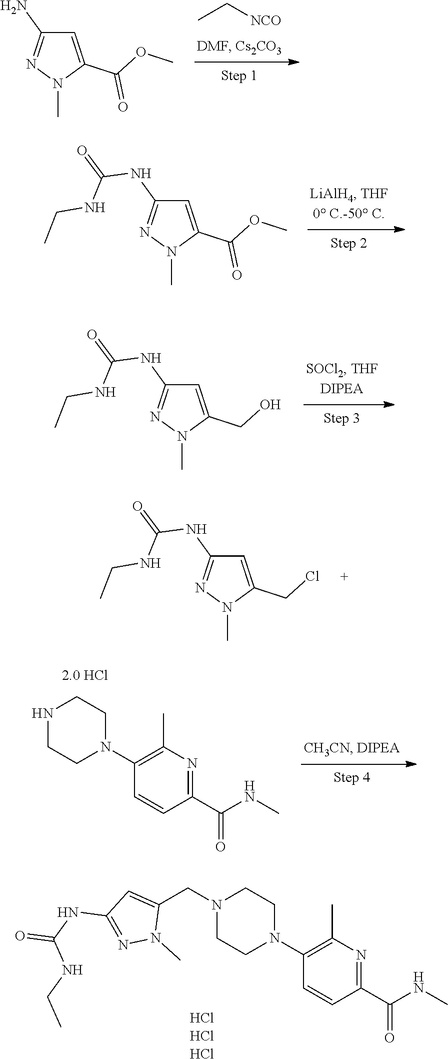

Step 1: Synthesis of methyl 3-(3-ethylureido)-1-methyl-1H-pyrazole-5-carboxylate: To a stirred solution of methyl 3-amino-1-methyl-1H-pyrazole-5-carboxylate (400 mg, 2.58 mmol) in DMF (4 mL) were added ethyl isocyanate (202 mg, 2.84 mmol) and Cs₂CO₃ (841 mg, 2.58 mmol) and the reaction mixture was stirred at 20° C. for 4 h. The reaction was quenched with water and extracted with EtOAc (50 mL×2). The combined organic layers were washed with brine, dried over Na₂SO₄, filtered and the filtrate was concentrated under reduced pressure. The residue was purified by silica gel chromatography to afford the title compound (330 mg, 57%). LCMS (m/z): 226.9 [M+H]⁺.

Step 2: Synthesis of 1-ethyl-3-(5-(hydroxymethyl)-1-methyl-1H-pyrazol-3-yl)urea: To a stirred solution of methyl 3-(3-ethylureido)-1-methyl-1H-pyrazole-5-carboxylate (300 mg, 1.33 mmol) in THF (4 mL) at 0° C. was added a 1M solution of LiAlH₄ in THF (1.6 mL, 1.59 mmol) dropwise and the reaction mixture was stirred at 20° C. for 2 h. The reaction was quenched with Na₂SO₄·10H₂O, filtered and the filtrate was concentrated under reduced pressure. The residue was purified by silica gel chromatography to afford the title compound (200 mg, 76%). LCMS (m/z): 199.1 [M+H]⁺.

Step 3: Synthesis of 1-(5-(chloromethyl)-1-methyl-1H-pyrazol-3-yl)-3-ethylurea: To stirred a solution of 1-ethyl-3-(5-(hydroxymethyl)-1-methyl-1H-pyrazol-3-yl)urea (50 mg, 0.25 mmol) and DIPEA (32 mg, 0.25 mmol) in THF (3 mL) at 0° C. was added a solution of SOCl₂ (36 mg, 0.30 mmol) dropwise and the reaction mixture was stirred at 20° C. for 1 h. The reaction was concentrated under reduced pressure to afford the title compound (65 mg, crude). LCMS (m/z): 217.1 [M+H]⁺.

Step 4: Synthesis of 5-(4-((3-(3-ethylureido)-1-methyl-1H-pyrazol-5-yl)methyl) piperazin-1-yl)-N,6-dimethylpicolinamide trihydrochloride: A mixture of 1-(5-(chloromethyl)-1-methyl-1H-pyrazol-3-yl)-3-ethylurea (65 mg, 0.30 mmol), N,6-dimethyl-5-(piperazin-1-yl) picolinamide dihydrochloride (92 mg, 0.30 mmol) and DIPEA (194 mg, 1.5 mmol) in CH₃CN (3 mL) was stirred at 50° C. for 16 h. The reaction was concentrated under reduced pressure and the residue was purified by prep-HPLC to afford the title compound (27 mg, 17%). LCMS (m/z): 415.0 [M+H]⁺; ¹H NMR (400 MHz, CD₃OD) δ 8.08-8.05 (m, 1H), 7.90-7.85 (m, 1H), 6.54 (s, 1H), 4.60 (s, 2H), 3.92 (s, 3H), 3.80-3.63 (m, 2H), 3.63-3.43 (m, 4H), 3.28 (q, J=7.2 Hz, 4H), 2.99 (s, 3H), 2.72 (d, J=3.5 Hz, 3H), 1.18 (t, J=7.2 Hz, 3H).

### Example S-53: Synthesis of 1-(6-((4-(6-(JH-imidazol-2-yl)-2-methylpyridin-3-yl)piperazin-1-yl)methyl)-5-fluoropyrimidin-4-yl)-3-ethylurea formate (Compound 503)

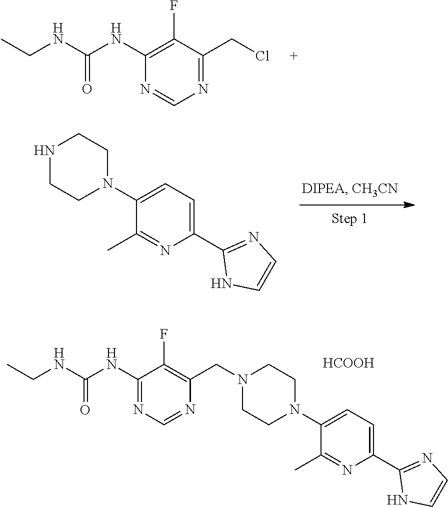 [SMILES: CCNC(=O)Nc1ncnc(CCl)c1F ; Cc1nc(-c2ncc[nH]2)ccc1N1CCNCC1 ; CCNC(=O)Nc1ncnc(CN2CCN(c3ccc(-c4ncc[nH]4)nc3C)CC2)c1F ; "HCOOH"]

Step 1: Synthesis of 1-(6-((4-(6-(1H-imidazol-2-yl)-2-methylpyridin-3-yl)piperazin-1-yl)methyl)-5-fluoropyrimidin-4-yl)-3-ethylurea formate: A mixture of 1-(6-(chloromethyl)-5-fluoropyrimidin-4-yl)-3-ethylurea (60 mg, 0.26 mmol), 1-(6-(1H-imidazol-2-yl)-2-methylpyridin-3-yl)piperazine (63 mg, 0.26 mmol) and DIPEA (67 mg, 0.52 mmol) in CH₃CN (4 mL) was stirred at 50° C. for 20 h. The reaction mixture was quenched with water and extracted with EtOAc (20 mL×3). The combined organic layers were washed with brine, dried over Na₂SO₄, filtered and the filtrate was concentrated under reduced pressure. The residue was purified by prep-HPLC to afford the title compound (30 mg, 24%). LCMS (m/z): 440.2 [M+H]⁺; ¹H NMR (400 MHz, CD₃OD) δ 9.26 (br s, 1H), 8.57 (s, 1H), 8.19 (s, 1H), 7.81 (d, J=8.3 Hz, 1H), 7.51 (d, J=8.4 Hz, 1H), 7.25 (s, 2H), 3.83 (s, 2H), 3.44-3.34 (m, 2H), 3.12-2.97 (m, 4H), 2.91-2.74 (m, 4H), 2.57 (s, 3H), 1.23 (t, J=7.2 Hz, 3H).

### Example S-54: Synthesis of 1-ethyl-3-(S-fluoro-6-((4-(2-methyl-6-(1H-pyrazol-1-yl)pyridin-3-yl)piperazin-1-yl)methyl)pyrimidin-4-yl)urea 2,2,2-trifluoroacetate (Compound 300)

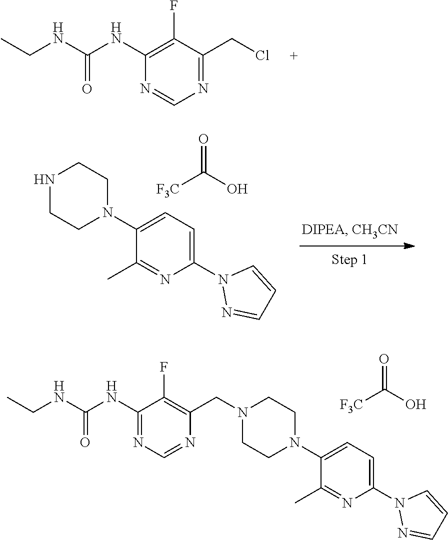

Step 1: Synthesis of 1-ethyl-3-(5-fluoro-6-((4-(2-methyl-6-(1H-pyrazol-1-yl)pyridin-3-yl)piperazin-1-yl)methyl)pyrimidin-4-yl)urea 2,2,2-trifluoroacetate: A mixture of 1-(6-(chloromethyl)-5-fluoropyrimidin-4-yl)-3-ethylurea (50 mg, 0.21 mmol), 1-(2-methyl-6-(1H-pyrazol-1-yl)pyridin-3-yl)piperazine 2,2,2-trifluoroacetate (120 mg, crude) and DIPEA (135.7 mg, 1.05 mmol) in CH₃CN (5 mL) was stirred at 30° C. for 16 h. The reaction mixture was quenched with water and extracted with EtOAc (30 mL×2). The combined organic layers were washed with brine, dried over Na₂SO₄, filtered and the filtrate was concentrated under reduced pressure. The residue was purified by prep-HPLC to afford the title compound (12 mg, 10%). LCMS (m/z): 440.2 [M+H]⁺; ¹H NMR (400 MHz, CD₃OD) δ 8.68 (s, 1H), 8.54 (d, J=2.4 Hz, 1H), 7.76-7.66 (m, 3H), 6.51 (d, J=2.1 Hz, 1H), 4.69 (s, 2H), 3.76-3.55 (m, 4H), 3.39 (q, J=7.2 Hz, 2H), 3.33-3.26 (m, 4H), 2.58 (s, 3H), 1.23 (t, J=7.2 Hz, 3H).

### Example S-55: Synthesis of 5-(4-((6-(3-ethylureido)pyridin-3-yl)methyl)piperazin-1-yl)-N,6-dimethylpicolinamide (Compound 481)

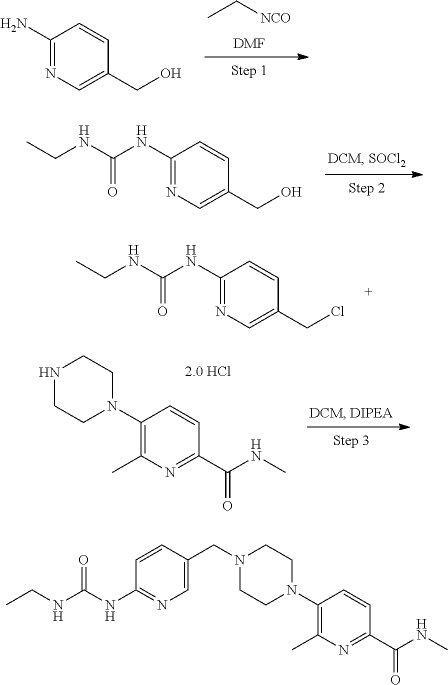

Step 1: Synthesis of 1-ethyl-3-(5-(hydroxymethyl)pyridin-2-yl)urea: To a stirred solution of (6-aminopyridin-3-yl)methanol (260 mg, 2.09 mmol) in DMF (4 mL) was added ethyl isocyanate (172 mg, 2.42 mmol) and the reaction was stirred at 20° C. for 20 h. The reaction mixture was quenched with water and extracted with EtOAc (30 mL×2). The combined organic layers were washed with brine, dried over Na₂SO₄, filtered and the filtrate was concentrated under reduced pressure. The residue was purified by silica gel chromatography to afford the title compound (150 mg, 37%). LCMS (m/z): 196.0 [M+H]⁺.

Step 2: Synthesis of 1-(5-(chloromethyl)pyridin-2-yl)-3-ethylurea: To a stirred solution of 1-ethyl-3-(5-(hydroxymethyl)pyridin-2-yl)urea (150 mg, 0.77 mmol) in DCM (4 mL) at 0° C. was added SOCl₂ (274 mg, 2.30 mmol) and the reaction mixture was stirred at 25° C. for 3 h. The reaction mixture was concentrated under reduced pressure to afford the title compound (120 mg, crude). LCMS (m/z): 214.1 [M+H]⁺.

Step 3: Synthesis of 5-(4-((6-(3-ethylureido)pyridin-3-yl)methyl)piperazin-1-yl)-N,6-dimethylpicolinamide: To a stirred solution of N,6-dimethyl-5-(piperazin-1-yl) picolinamide dihydrochloride (236 mg, crude) in DCM (10 mL) was added DIPEA (496 mg, 3.84 mol) and the reaction was stirred at 25° C. for 10 min. Then 1-(5-(chloromethyl)pyridin-2-yl)-3-ethylurea (120 mg, crude) was added to the reaction mixture and stirred at 25° C. for 17 h. The reaction mixture was quenched with water and extracted with DCM (20 mL×2). The combined organic layers were dried over Na₂SO₄, filtered and the filtrate was concentrated under reduced pressure. The residue was purified by prep-HPLC to afford the title compound (6 mg, 1.9%). LCMS (m/z): 412.2 [M+H]⁺; ¹H NMR (400 MHz, CD₃OD) δ 8.20 (s, 1H), 7.86 (d, J=8.3 Hz, 1H), 7.73 (dd, J=8.5, 1.7 Hz, 1H), 7.49 (d, J=8.3 Hz, 1H), 7.11 (d, J=8.5 Hz, 1H), 3.71 (s, 2H), 3.37-3.32 (m, 2H), 3.12-3.02 (m, 4H), 2.94 (s, 3H), 2.88-2.75 (m, 4H), 2.54 (s, 3H), 1.21 (t, J=7.2 Hz, 3H).

It is understood that compounds disclosed herein are synthesized using the general synthetic schemes 1 to 12 or using the experimental details as described above. The steps involved in the synthetic routes are clearly familiar to those skilled in the art, wherein the substituents described in the formulae disclosed herein can be varied with a choice of appropriate starting materials and reagents utilized in the steps presented.

### Example B1. PARP1 Fluorescence Polarization Binding Assay

Compounds of the present disclosure were tested in a PARP1 fluorescence polarization binding assay at Pharmaron (Beijing, P.R. China). In this biochemical assay, binding of test compound to recombinant human PARP1 reduces the amount of binding of a fluorescent probe, and consequently reduces the detected level of fluorescence polarization. Test compound stock solutions were prepared in DMSO and then serially diluted into 10 concentrations by 3-fold dilution in a 384-well plate using a TECAN EVO200. Alternatively, a single concentration was prepared. 60 nL of each dilution was transferred to the plate using an Echo550 (Labcyte), and then 10 μL 40 nM GST-tagged PARP1 (BPS Bioscience, Cat #80501), diluted in assay buffer (50 mM Tris pH 8.0, 0.001% Triton X-100, 10 mM MgCl2, 150 mM NaCl), was added to the plate. After a centrifugation at 1000 rpm for 1 min, the assay plate was incubated for 30 min at RT. 10 μL 6 nM PARP1-FL (TOCRIS, Cat #6461), diluted using assay buffer, was then added to the plate. The final concentrations of PARP1 and PARP1-FL were 20 nM and 3 nM, respectively, in a total volume of 20 μL. After a centrifugation at 1000 rpm for 1 min, the assay plate was incubated for 4 h at RT. Samples in each well were read using an Envision instrument (Perkin-Elmer; Ex=480 nm Em=FITC FP-P pol 535 nm & FITC FP-S pol 535 nm). Percent inhibition was calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. Binding IC50 values were calculated using XLFit (equation 201: y=A+((B−A)/(1+((x/C)\hat{ }D))), where A=bottom, B=top, C=IC50, and D=slope) with a floating top and bottom for curves. Binding IC50 values are shown in Table A and are presented in ranges, in which “+++”<100 nM, 100 nM≤“++”<3000 nM, and “+”≥3000 nM.

TABLE A

| | | PARP2 % |
|---|---|---|
| Example | PARP1 | inh at |
| No. | IC₅₀ | 10 μM |
| 3 | + | |
| 7 | +++ | * |
| 9 | +++ | * |
| 13 | +++ | * |
| 21 | +++ | * |
| 22 | +++ | ** |
| 24 | +++ | * |
| 29 | ++ | * |
| 30 | +++ | * |
| 31 | +++ | * |
| 42 | +++ | * |
| 46 | +++ | * |
| 50 | +++ | * |
| 70 | +++ | * |
| 81 | +++ | * |
| 82 | +++ | * |
| 105 | + | * |
| 161 | ++ | * |
| 202 | ++ | * |
| 229 | +++ | * |
| 230 | +++ | * |
| 242 | +++ | * |
| 246 | +++ | * |
| 257 | +++ | ** |
| 259 | +++ | * |
| 260 | +++ | * |
| 261 | +++ | * |
| 262 | +++ | * |
| 263 | +++ | * |
| 264 | +++ | ** |
| 267 | +++ | * |
| 268 | +++ | * |
| 269 | +++ | * |
| 272 | +++ | * |
| 273 | +++ | * |
| 274 | +++ | * |
| 275 | +++ | * |
| 276 | +++ | * |
| 277 | +++ | * |
| 278 | +++ | * |
| 279 | +++ | |
| 280 | + | |
| 281 | +++ | |

### Example B2. PARP2 Fluorescence Polarization Binding Assay

Compounds of the present disclosure were tested in a PARP2 fluorescence polarization binding assay at Pharmaron (Beijing, P.R. China). In this biochemical assay, binding of test compound to recombinant human PARP2 reduces the amount of binding of a fluorescent probe, and consequently reduces the detected level of fluorescence polarization. Test compound stock solutions were prepared in DMSO and then serially diluted into 10 concentrations by 3-fold dilution in a 384-well plate using a TECAN EVO200. 60 nL of each dilution was transferred to the plate using an Echo550 (Labcyte), and then 10 μL 40 nM GST-tagged PARP2 (BPS Bioscience, Cat #80502), diluted in assay buffer (50 mM Tris pH 8.0, 0.001% Triton X-100, 10 mM MgCl2, 150 mM NaCl), was added to the plate. After a centrifugation at 1000 rpm for 1 min, the assay plate was incubated for 30 min at RT. 10 μL 6 nM PARP1-FL (TOCRIS, Cat #6461), diluted using assay buffer, was then added to the plate. The final concentrations of PARP2 and PARP1-FL were 20 nM and 3 nM, respectively, in a total volume of 20 μL. After a centrifugation at 1000 rpm for 1 min, the assay plate was incubated for 4 h at RT. Samples in each well were read using an Envision instrument (Perkin-Elmer; Ex=480 nm Em=FITC FP-P pol 535 nm & FITC FP-S pol 535 nm). Percent inhibition was calculated from mP values using Inhibition (%)=[1−(mP_{c} mP_{L})/(mP_{H}−mP_{L})]×100%, where mP_{c}, mP_{L}, and mP_{H} are the mP values of test compound, Low controls, and High controls, respectively. Binding IC₅₀ values are calculated using XLFit (equation 201: y=A+((B−A)/(1+((x/C)\hat{ }D))), where A=bottom, B=top, C=IC₅₀, and D=slope) with a floating top and bottom for curves. Values for percent inhibition at 10 μM are shown in Table A, and are presented in ranges, in which “***”≥67%, 33%≤“**”<67%, and “*”<33%.

### Example B3. Cell Viability Assay

Compounds of the present disclosure were tested for their effects on the viability of cancer cells. DLD-1 wild-type and DLD-1 BRCA2(−/−) colorectal adenocarcinoma cells, an isogenic pair of cell lines differing in the presence and absence, respectively, of both BRCA2 alleles, were used. The cells were harvested during the logarithmic growth period, counted, and seeded at 50 cells/well for DLD-1 wild-type and 200 cells/well for DLD-1 BRCA2(−/−) in a 384-well cell culture plate. After seeding, cells were incubated at 37° C., 5% CO₂ overnight. Cells were treated with serially diluted test compounds at 10 concentrations (e.g., from 1.5 nM-30 μM) for generation of dose response curves, or at a single concentration. The plate was further incubated for another 7 days in a humidified incubator at 37° C. and 5% CO₂. Cell viability was assessed by luminescence measurement after addition of Cell Titer-Glo reagent (Promega, Madison, Wis.) according to the manufacturer's instructions. Cell viability IC₅₀ values were calculated using XLFit, equation 201. y=A+((B−A)/(1+((x/C)\hat{ }D))), where A=bottom, B=top, C=IC₅₀, and D=slope. IC₅₀ values are shown in Table B and are presented in ranges, in which “+++”<300 nM, 300 nM≤“++”<3000 nM, and “+”≥3000 nM. Values for percent inhibition at 10 μM are also shown in Table B and are presented in ranges, in which “***”≥67%, 33%: “**”≤67%, and “*”<33%. Effects of the test compounds on the viability of other cell lines such as MDA-MB-436, MDA-MB-231, SUM149PT, HCC1395, and UWB1.289 are determined in an analogous method.

TABLE B

| | | DLD-1 |
|---|---|---|
| | DLD-1 | wild- |
| | BRCA2 | type % |
| Example | (−/−) | inh at |
| No. | IC₅₀ | 10 μM |
| 7 | ++ | ** |
| 9 | +++ | * |
| 13 | +++ | * |
| 21 | +++ | * |
| 22 | +++ | * |
| 24 | +++ | * |
| 29 | + | * |
| 30 | ++ | * |
| 31 | ++ | * |
| 42 | +++ | * |
| 46 | +++ | * |
| 50 | +++ | * |
| 70 | +++ | * |
| 81 | +++ | * |
| 82 | +++ | * |
| 161 | ++ | * |
| 202 | + | * |
| 229 | +++ | * |
| 230 | +++ | * |
| 242 | ++ | * |
| 246 | +++ | * |
| 257 | ++ | * |
| 259 | ++ | * |
| 260 | ++ | * |
| 261 | +++ | * |
| 262 | +++ | * |
| 263 | +++ | * |
| 264 | +++ | * |
| 267 | +++ | * |
| 268 | +++ | * |
| 269 | +++ | * |
| 272 | +++ | * |
| 273 | + | * |
| 274 | +++ | * |
| 275 | +++ | * |
| 276 | +++ | * |
| 277 | + | * |
| 278 | +++ | * |

### Example B4. PARP1 Biochemical Trapping Assay

Compounds of the present disclosure are tested in a PARP1 biochemical trapping assay at BPS Bioscience (San Diego, CA). PARP1 is known to bind damaged DNA through its DNA-binding domains. Binding to DNA activates PARP1, and in the presence of NAD+, PARP1 ribosylates itself (auto-ribosylation), leading to PARP1 dissociation from the DNA due to the accumulated negative charge of the ribosyl polymer. In the presence of some inhibitors, however, PARP remains bound to the DNA, a phenomenon termed trapping. Trapped PARP-DNA complexes have been shown to be highly cytotoxic to cancer cells.

The BPS PARPtrap assay kit for PARP1 (BPS Catalog #80584) is used. A series of dilutions of the compounds is prepared with 10/DMSO in water. The final concentration of DMSO is 1% in all reactions. The enzymatic reactions are conducted in duplicates at room temperature in a 96-well plate. The 45 μl reaction mixtures in PARPtrap buffer 1 containing PARP1 fluorescent labeled oligonucleotide duplex, PARP enzyme and the test compound are incubated at room temperature for 10 min. After pre-incubation, the enzymatic reaction is initiated by adding 5 μl of 10×NAD+ solution. The reaction is incubated for 45-60 min at room temperature.

Fluorescence polarization is measured at an excitation of 470 nm and an emission of 518 nm using a Tecan Infinite M1000 microplate reader. The blank value is subtracted from all other values. Fluorescence polarization is analyzed by Tecan Magellan 6 software and wells containing DNA are used as references. The fluorescence polarization data are then evaluated using GraphPad Prism software. The fluorescence polarization in absence of the compound in each data set is defined as 0% activity (FPₒ), while the fluorescence polarization in the absence of both NAD+ and the compound is defined as 100% activity (FPₜ). The percent activity in the presence of the compound is calculated according to the equation % activity=[FP−FPₒ]/[(FPₜ−FPₒ)]−100, where FP=the fluorescence polarization in the presence of the compound.

The values of % activity versus a series of compound concentrations are then plotted using non-linear regression analysis of a sigmoidal dose-response curve generated with the equation Y=B+(T−B)/1+10^{((-LogEC50-X)×Hill Slope}>, where Y=percent activity, B=minimum percent activity, T=maximum percent activity, X=logarithm of compound and Hill Slope=slope factor or Hill coefficient.

### Example B5. PARP2 Biochemical Trapping Assay

Compounds of the present disclosure are tested in a PARP2 biochemical trapping assay at BPS Bioscience (San Diego, CA). PARP2 recognizes and binds damaged DNA through its DNA binding domain. Binding to DNA activates PARP2, and in the presence of NAD⁺, PARP2 ribosylates itself (auto-ribosylation), leading to PARP2 dissociation from the DNA due to the accumulated negative charge of the ribosyl polymer. In the presence of some inhibitors, however, PARP remains bound to the DNA, a phenomenon termed trapping. Trapped PARP-DNA complexes have been shown to be highly cytotoxic to cancer cells.

The BPS PARPtrap assay kit for PARP2 (BPS Catalog #78296) is used. Dilutions of the compounds are prepared with 10% DMSO in water. The final concentration of DMSO is 1% in all reactions. The enzymatic reactions are conducted in duplicates at room temperature in a 96-well plate. The 45 μl reaction mixtures in PARPtrap buffer specific for PARP fluorescent labelled oligonucleotide duplex, PARP enzyme and the test compound are incubated at room temperature for 10 min. After pre-incubation, the enzymatic reaction is initiated by adding 5 μl of 10×NAD⁺ solution. The reaction is incubated for 45-60 min at room temperature.

Fluorescence polarization is measured at an excitation of 470 nm and an emission of 518 nm using a Tecan Infinite M1000 microplate reader. The blank value is subtracted from all other values. Fluorescence polarization is analyzed by Tecan Magellan 6 software and wells containing DNA are used as references. The fluorescence polarization data are then evaluated using GraphPad Prism software. The fluorescence polarization in absence of the compound in each data set is defined as 0% activity (FPₒ), while the fluorescence polarization in the absence of both NAD⁺ and the compound is defined as 100% activity (FPₜ). The percent activity in the presence of the compound is calculated according to the equation % activity=[FP−FPₒ]/[(FPₜ−FPₒ)]×100, where FP=the fluorescence polarization in the presence of the compound.

### Example B6. Colony Formation Assay

A colony formation assay is performed to assess the activity of test compounds. MDA-MB-436 cells are harvested, counted and seeded in culture medium (DMEM+10% FBS+1% PS) at a specified density in a volume of 600 μL per well in a 24-well cell culture plate. The plates are incubated overnight at 37° C. in a 5% CO₂ incubator. Test compounds are dissolved in DMSO and added to the plate. At regular intervals, for example 4-6 days, the supernatant is removed and 1000 μL of freshly diluted compounds are added to the plates. The plates are returned to incubator to continue incubation. After treatment with the compounds for a specified number of days, e.g. 10-14 days, detection is performed. For detection, the plate is incubated 20 min after removing the medium and adding fixing solution. Subsequently, the fixing solution is removed and the plates washed with PBS. The plates are then stained with staining solution (0.1% (w/v) crystal violet). After removal of the staining solution, the plates are washed with PBS and scanned on a LICOR imager using the 700 nm channel. The inhibition activity is calculated with the equation % inhibition=(1−LUM_{cmpd}/LUM_{Ave_HC})×100 where LUM_{cmpd} is raw data of compounds minus average signal of blank and LUM_{Ave_HC} is average signal of DMSO raw data minus average signal of blank.

### Exemplary Embodiments

Exemplary Embodiment No. 1. A compound of Formula (I):

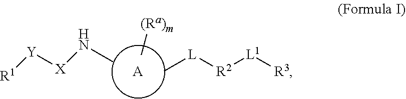

or a pharmaceutically acceptable salt, thereof, wherein
\textcircled{A} is C₃₋₁₀ cycloalkylene, C₆₋₁₀ arylene, 3- to 10-membered heterocycloalkylene, or 5- to 10-membered heteroarylene;
m is 0, 1, 2, 3, 4, or 5;
each R^{a} independently is halogen, —CN, —OH, —NH₂, —O—(C₁₋₆alkyl), —NH(C₁₋₆alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆ alkyl), —NH(C₁₋₆ alkyl), —N(C₁₋₆ alkyl)₂, or —C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆- to aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{a}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₁₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
X is —C(═O)—, —S(═O)₂—;
Y is a bond, —O—, —S—, —NH—, or C₁₋₆ alkylene, wherein the C₁₋₆ alkylene is optionally substituted with one or more oxo, halogen, —CN, —OH, —NH₂, C₁₋₆ alkyl, or two C₁₋₆ alkyl that combine, with the atom or atoms to which they are attached, to form a C₃₋₁₀ cycloalkyl group;
R¹ is H, halogen, —CN, —OH, —NH₂, —C₁₋₆ alkyl, —O—(C₁₋₆ alkyl), —NH(C₁₋₆ alkyl), —N(C₁₋₆ alkyl)(C₂₋₆ alkyl), C₃₋₁₀ cycloalkyl, C₆₋₁₀ aryl, 3- to 10-membered heterocycloalkyl, or 5- to 10-membered heteroaryl, wherein the —C₁₋₆ alkyl, —O—(C₁₋₆ alkyl), —NH(C₁₋₆ alkyl), or —N(C₁₋₆ alkyl)₂, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, or 5- to 10-membered heteroaryl, and the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
L is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₁₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R² is C₃₋₁₀ cycloalkylene, C₆- to arylene, 3- to 10-membered heterocycloalkylene, or 5- to 10-membered heteroarylene, wherein the C₃₋₁₀ cycloalkylene, C₆₋₁₀ arylene, 3- to 10-membered heterocycloalkylene, or 5- to 10-membered heteroarylene is optionally substituted with one or more R^{b};
each R^{b} independently is halogen, —CN, —OH, —NH₂, —O—(C₁₋₆alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{b} combine to form oxo, wherein the —O—(C₁₋₆alkyl), —NH(C₁₋₆alkyl), —N(C₁₋₆alkyl)₂, C₁₋₆alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C_{1- 6} alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{b}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
L¹ is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R³ is C₃₋₁₀ cycloalkyl, C₆₋₁₀ aryl, 3- to 10-membered heterocycloalkyl, or 5- to 10-membered heteroaryl, wherein the C₃₋₁₀cycloalkyl, C₆₋₁₀ aryl, 3- to 10-membered heterocycloalkyl, or 5- to 10-membered heteroaryl is optionally substituted with one or more R^{c};
each R^{c} independently is halogen, —CN, —OH, —NH₂, —C(═O)—H, —C(═O)—NH₂, —NH—C(═O)—H, —NH—(C═O)—NH₂, —C(═O)—CN, —NH—C(═O)—CN, —C(═O)—NH—CN, —O—(C₁₋₆ alkyl), —NH(C₁₋₆ alkyl), —N(C₁₋₆ alkyl)₂, —C(═O)—(C₁₋₆alkyl), —C(═O)—NH—(C₁₋₆ alkyl), —NH—C(═O)—(C₁₋₆ alkyl), —NH—(C═O)—NH—(C₁₋₆alkyl), —NH—(C═O)—O—(C₁₋₆ alkyl), —C(═O)—NH—(C₃₋₁₀cycloalkyl), —C(═O)—NH-(3- to 10-membered heterocycloalkyl), C₁₋₆ alkyl, C₂₋₆ alkenyl, C₂₋₆ alkynyl, C₃₋₁₀cycloalkyl, C₆₋₁₀ aryl, 3- to 10-membered heterocycloalkyl, or 5- to 10-membered heteroaryl, or two R^{c} combine to form oxo, wherein the —O—(C₁₋₆ alkyl), —NH(C₁₋₆ alkyl), —N(C₁₋₆ alkyl)₂, —C(═O)—(C₁₋₆ alkyl), —C(═O)—NH—(C₁₋₆alkyl), —NH—C(═O)—(C₁₋₆ alkyl), —NH—(C═O)—NH—(C₁₋₆alkyl), or —NH—(C═O)—O—(C₁₋₆ alkyl), is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆- to aryl, or 5- to 10-membered heteroaryl;
or two R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
or one R^{b} and one R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R^{bc}; and
each R^{bc} independently is oxo, deuterium, halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.
   Exemplary Embodiment No. 2. The compound of Exemplary Embodiment No. 1, wherein the compound is of Formula II:

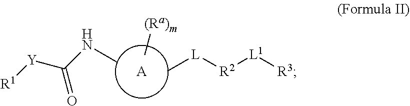

or a pharmaceutically acceptable salt thereof.
Exemplary Embodiment No. 3. The compound of Exemplary Embodiment No. 2, wherein the compound is of Formula II-a, II-b, II-c, II-d, 11-e, II-f, II-g, II-h, II-i, II-j, II-k, II-1, II-m, II-n, II-o, II-p, II-q, II-r, II-s, II-t, II-u, II-v, II-w, II-x, II-y, II-z, (II-aa), (II-ab), (II-ac), (II-ad), (II-ae), (II-af), (II-ag), (II-ah), (II-ai, II-aj, II-ak or II-al):

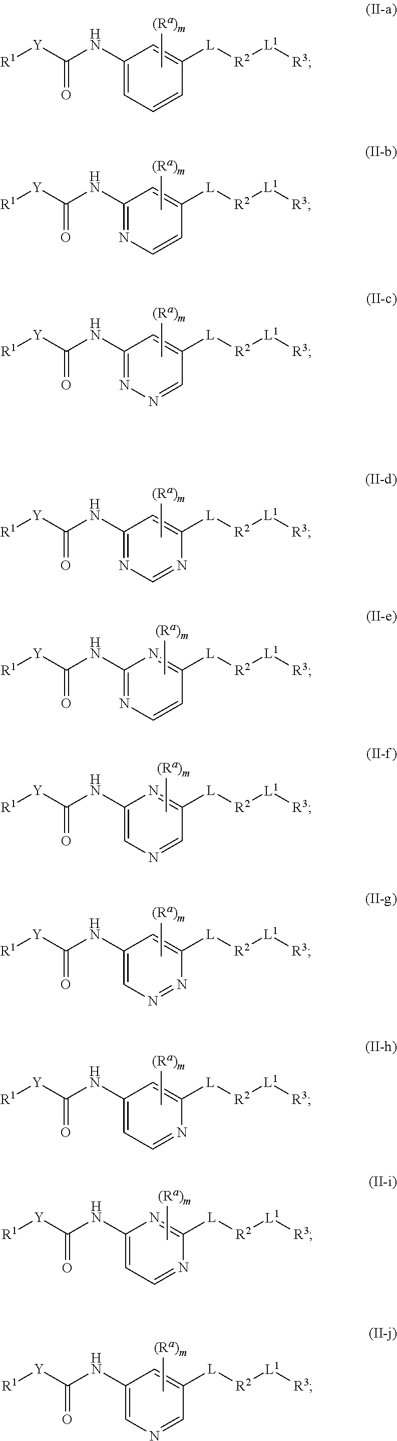

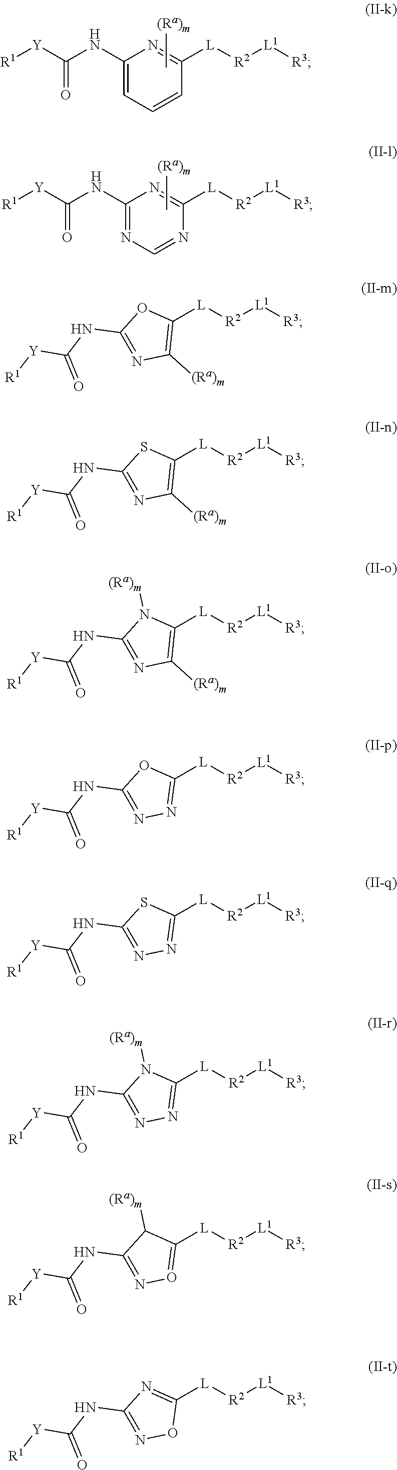

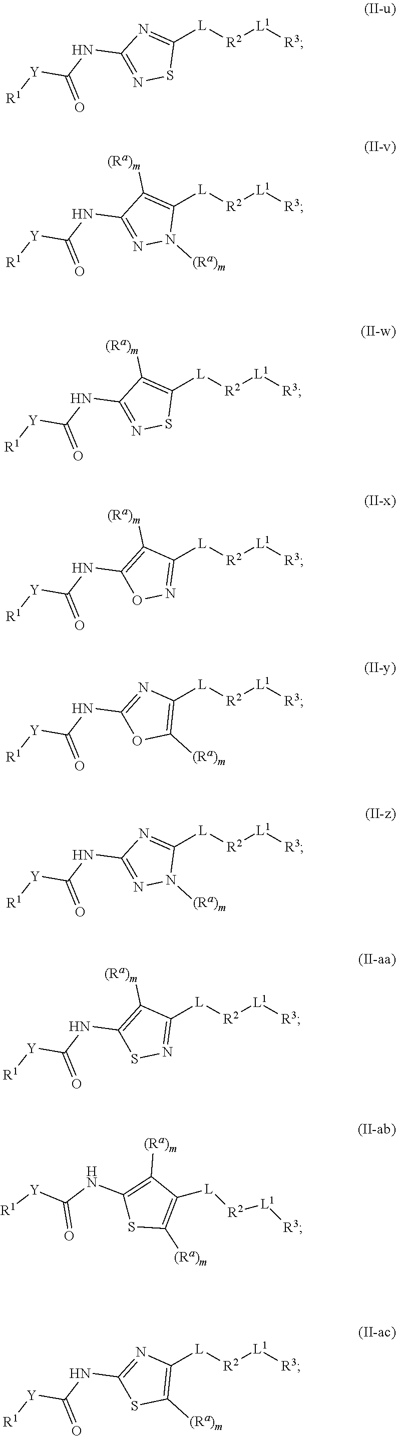

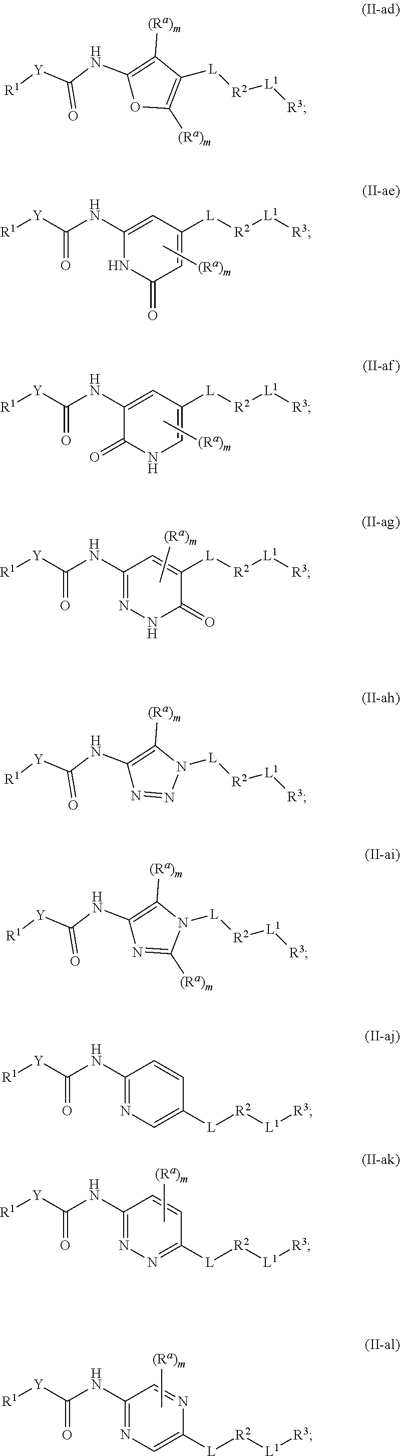

or a pharmaceutically acceptable salt thereof.
Exemplary Embodiment No. 4. The compound of Exemplary Embodiment No. 1, wherein the compound is of Formula III:

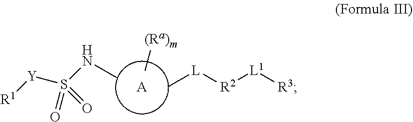

or a pharmaceutically acceptable salt thereof.
Exemplary Embodiment No. 5. The compound of Exemplary Embodiment No. 4, wherein the compound is of Formula III-a, III-b, III-c, III-d, III-e, III-f, III-g, III-h, III-i, III-j, III-k, III-l, III-m, III-n, III-o, III-p, III-q, III-r, III-s, III-t, III-u, III-v, III-w, III-x, III-y, III-z, (III-aa), (III-ab), (III-ac), (III-ad), (III-ae), (III-af), (III-ag), (III-ah), (III-ai), (III-aj), (III-ak), or (III-al):

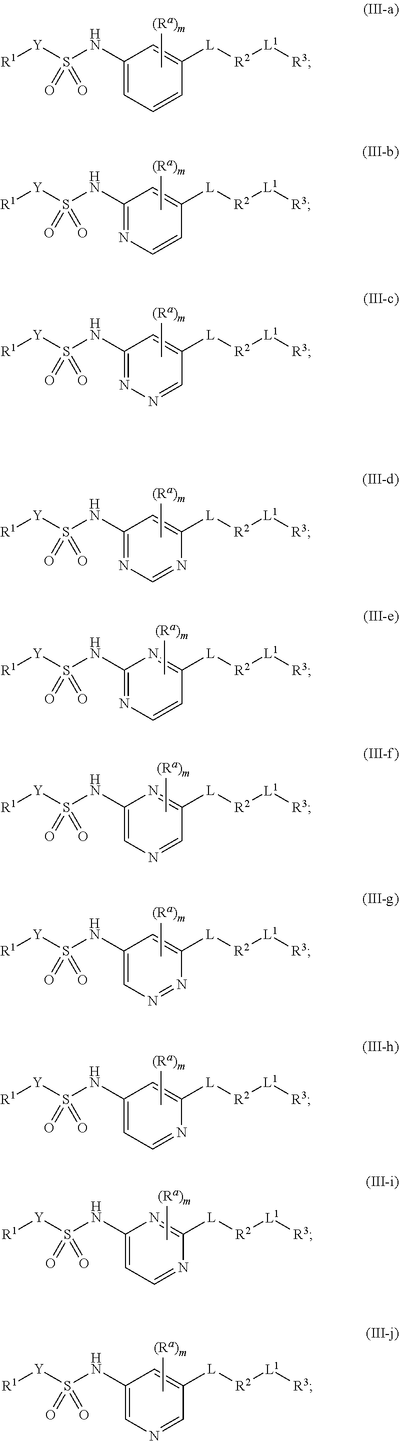

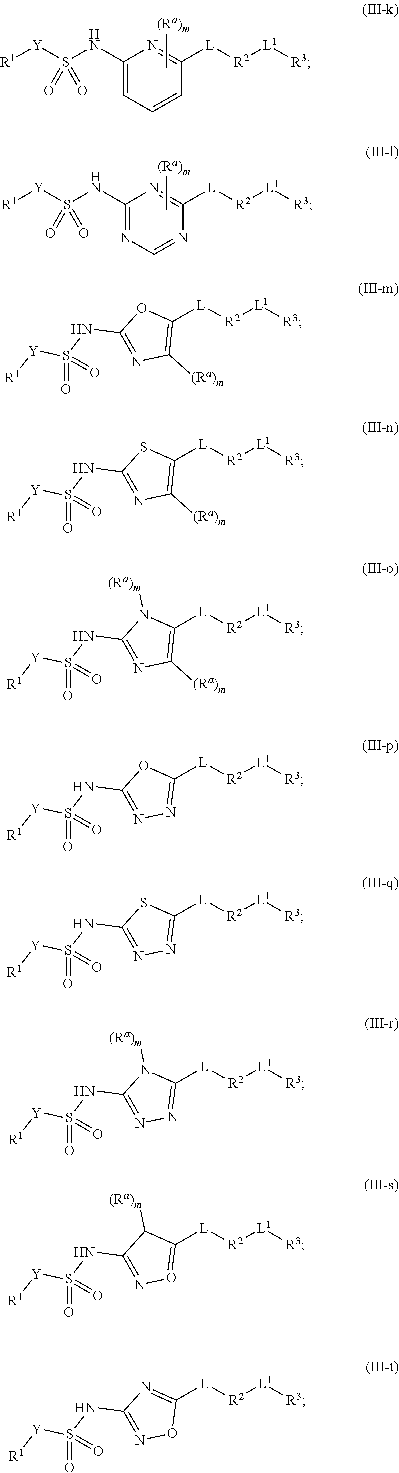

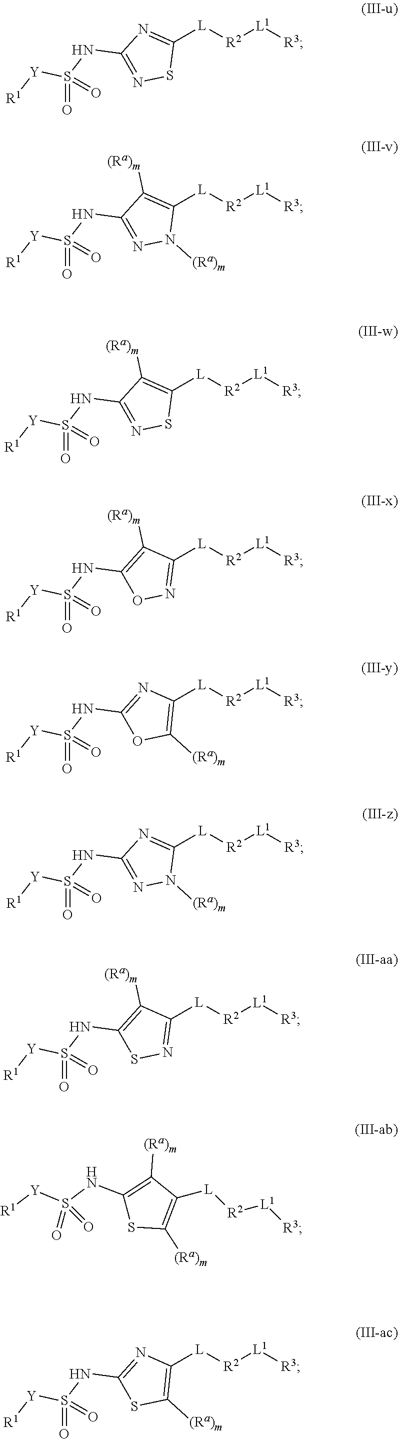

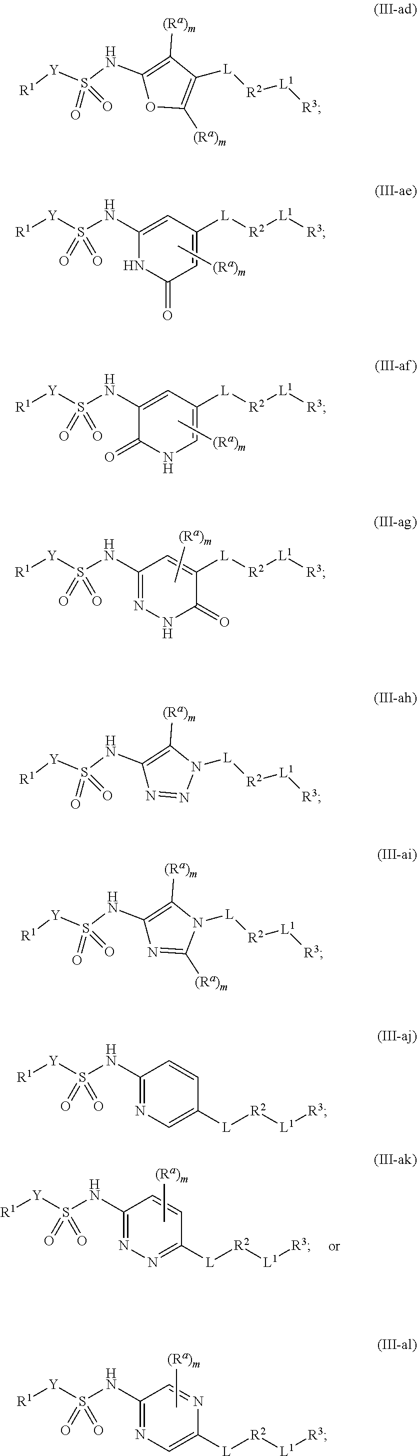

or a pharmaceutically acceptable salt thereof.
Exemplary Embodiment No. 6. The compound Exemplary Embodiment No. 1, wherein the compound is of Formula IV′:

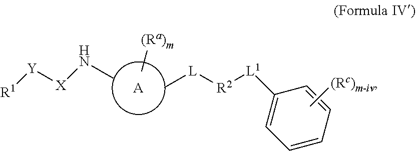

or a pharmaceutically acceptable salt thereof;
wherein m-iv is 0 to 5.
   Exemplary Embodiment No. 7. The compound of Exemplary Embodiment No. 1, wherein the compound is of Formula V′:

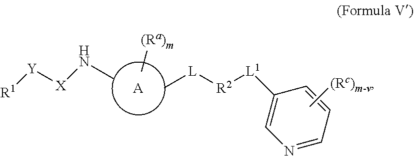

or a pharmaceutically acceptable salt thereof;
wherein m-v is 0 to 4.
   Exemplary Embodiment No. 8. The compound of Exemplary Embodiment No. 1, wherein the compound is of Formula VI′:

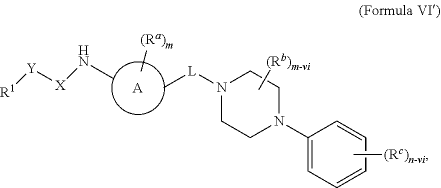

or a pharmaceutically acceptable salt thereof,
wherein m-vi is 0 to 8; and
n-vi is 0 to 5.
   Exemplary Embodiment No. 9. The compound of Exemplary Embodiment No. 1, wherein the compound is of Formula VII′:

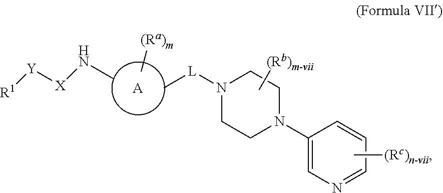

or a pharmaceutically acceptable salt thereof;
wherein m-vii is 0 to 8; and
n-vii is 0 to 4.
   Exemplary Embodiment No. 10. The compound of Exemplary Embodiment No. 1, wherein the compound is of Formula VIII′:

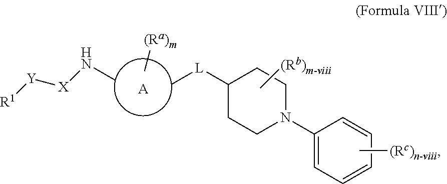

or a pharmaceutically acceptable salt thereof;
wherein m-viii is 0 to 8; and
n-viii is 0 to 5.
   Exemplary Embodiment No. 11. The compound of Exemplary Embodiment No. 1, wherein the compound is of Formula IX′:

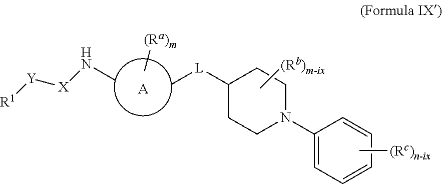

or a pharmaceutically acceptable salt thereof;
wherein m-ix is 0 to 9; and
n-ix is 0 to 4.
   Exemplary Embodiment No. 12. The compound of Exemplary Embodiment No. 1, wherein the compound is of Formula X′:

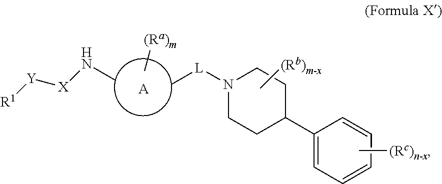

or a pharmaceutically acceptable salt thereof;
wherein m-x is 0 to 9; and
n-x is 0 to 5.
   Exemplary Embodiment No. 13. The compound of Exemplary Embodiment No. 1, wherein the compound is of Formula XI′:

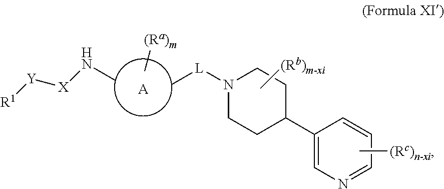

or a pharmaceutically acceptable salt thereof;
wherein m-xi is 0 to 9; and
n-xi is 0 to 4.
   Exemplary Embodiment No. 14. The compound of Exemplary Embodiment No. 1, wherein the compound is of Formula XII:

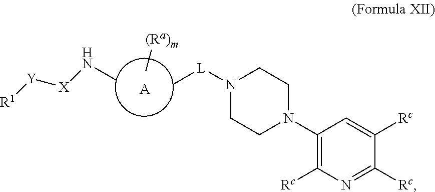

or a pharmaceutically acceptable salt thereof.
Exemplary Embodiment No. 15. The compound of Exemplary Embodiment No. 1, wherein the compound is of Formula XIII:

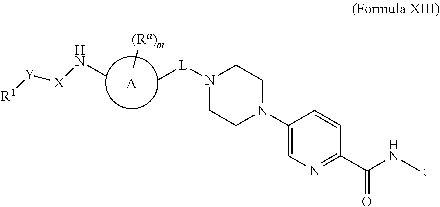

or a pharmaceutically acceptable salt thereof.
Exemplary Embodiment No. 16. The compound of Exemplary Embodiment No. 1, wherein the compound is of Formula XIV:

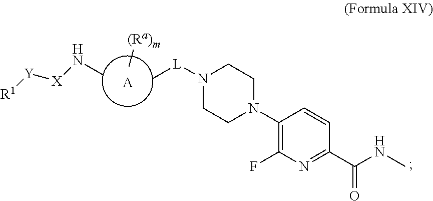

or a pharmaceutically acceptable salt thereof.
Exemplary Embodiment No. 17. The compound of Exemplary Embodiment No. 1, wherein the compound is of Formula XV:

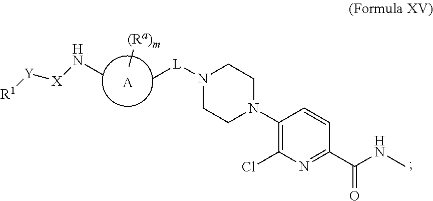

or a pharmaceutically acceptable salt thereof.
Exemplary Embodiment No. 18. The compound of Exemplary Embodiment No. 1, wherein the compound is of Formula XVI:

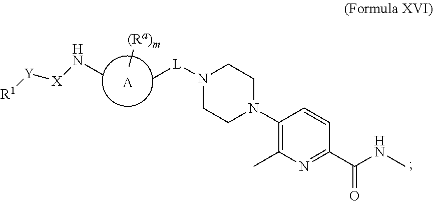

or a pharmaceutically acceptable salt thereof.
Exemplary Embodiment No. 19. The compound of Exemplary Embodiment No. 1, wherein the compound is of Formula XVII:

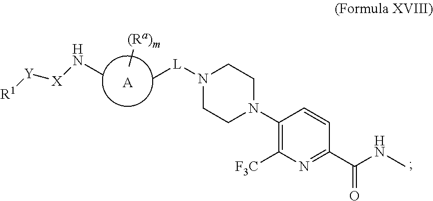

or a pharmaceutically acceptable salt thereof.
Exemplary Embodiment No. 20. The compound of Exemplary Embodiment No. 1, wherein the compound is of Formula XVIII:

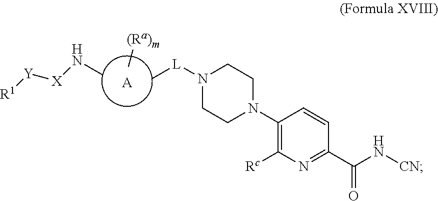

or a pharmaceutically acceptable salt thereof.
Exemplary Embodiment No. 21. The compound of Exemplary Embodiment No. 1, wherein the compound is of Formula XIX:

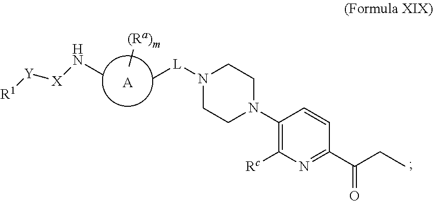

or a pharmaceutically acceptable salt thereof.
Exemplary Embodiment No. 22. The compound of Exemplary Embodiment No. 1, wherein the compound is of Formula XX:

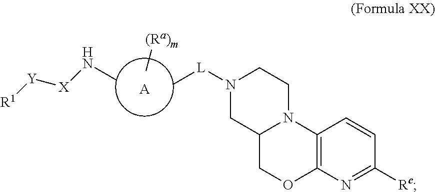

or a pharmaceutically acceptable salt thereof.
Exemplary Embodiment No. 23. The compound of Exemplary Embodiment No. 1, wherein the compound is of Formula XXI:

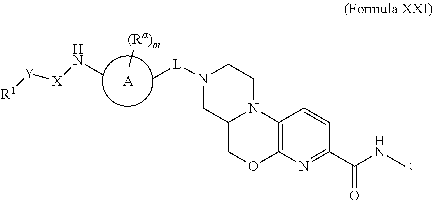

or a pharmaceutically acceptable salt thereof.
Exemplary Embodiment No. 24. The compound of Exemplary Embodiment No. 1, wherein the compound is of Formula XXII:

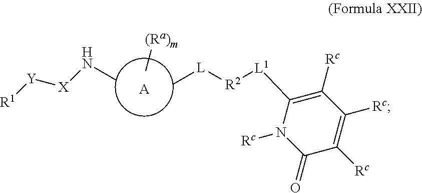

or a pharmaceutically acceptable salt thereof.
Exemplary Embodiment No. 25. The compound of Exemplary Embodiment No. 1, wherein the compound is of Formula XXIII′:

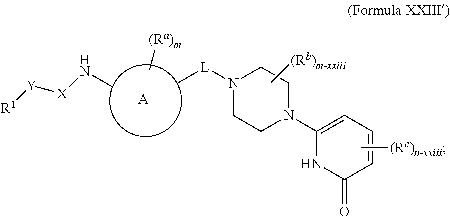

Or a pharmaceutically acceptable salt thereof;
wherein m-xxiii is 0 to 8; and
n-xxiii is 0 to 4.
   Exemplary Embodiment No. 26. The compound of Exemplary Embodiment No. 1, wherein the compound is of Formula XXIV:

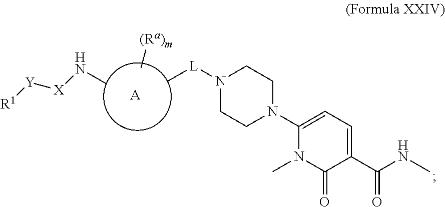

or a pharmaceutically acceptable salt thereof.
Exemplary Embodiment No. 27. The compound of Exemplary Embodiment No. 1, wherein the compound is of Formula XXV:

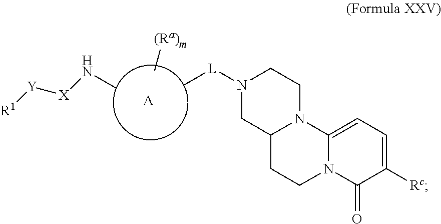

or a pharmaceutically acceptable salt thereof.
Exemplary Embodiment No. 28. The compound of Exemplary Embodiment No. 1, wherein the compound is of Formula XXVI:

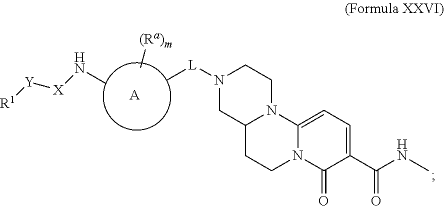

or a pharmaceutically acceptable salt thereof.
Exemplary Embodiment No. 29. The compound of Exemplary Embodiment No. 1, wherein the compound is of Formula XXVII:

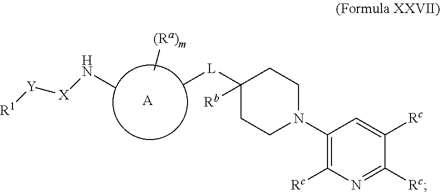

or a pharmaceutically acceptable salt thereof.
Exemplary Embodiment No. 30. The compound of Exemplary Embodiment No. 1, wherein the compound is of Formula XXVIII:

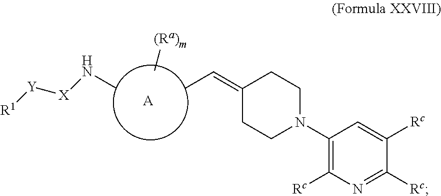

or a pharmaceutically acceptable salt thereof.
Exemplary Embodiment No. 31. The compound of Exemplary Embodiment No. 1, wherein the compound is of Formula XXIX:

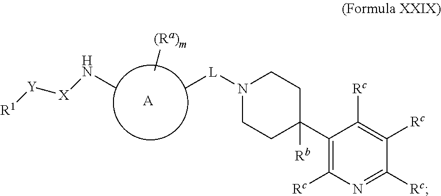

or a pharmaceutically acceptable salt thereof.
Exemplary Embodiment No. 32. The compound of Exemplary Embodiment No. 1, wherein the compound is of Formula XXX′:

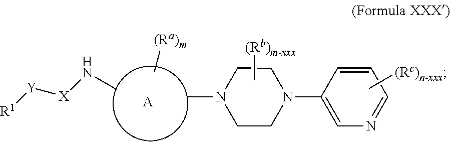

or a pharmaceutically acceptable salt thereof;
wherein m-xxx is 0 to 8; and
n-xxx is 0 to 4.
   Exemplary Embodiment No. 33. The compound of Exemplary Embodiment No. 1, wherein the compound is of Formula XXXI′:

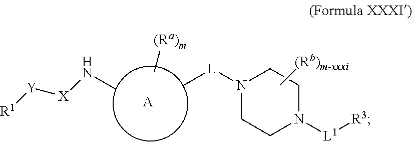

Or a pharmaceutically acceptable salt thereof;
wherein m-xxxi is 0 to 8.
   Exemplary Embodiment No. 34. The compound of Exemplary Embodiment No. 1, wherein the compound is of Formula XXXII′:

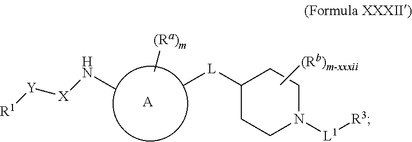

or a pharmaceutically acceptable salt thereof;
wherein m-xxxii is 0 to 9.
   Exemplary Embodiment No. 35. The compound of Exemplary Embodiment No. 1, wherein the compound is of Formula XXXIII′:

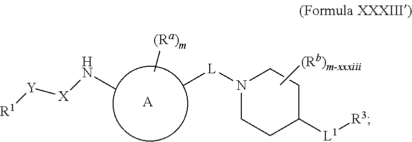

or a pharmaceutically acceptable salt thereof;
wherein m-xxxiii is 0 to 9.
   Exemplary Embodiment No. 36. The compound of Exemplary Embodiment No. 1, wherein the compound is of Formula XXXIV:

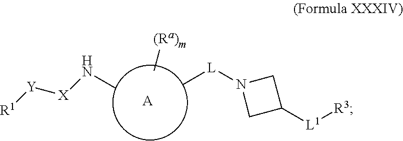

or a pharmaceutically acceptable salt thereof.

Exemplary Embodiment No. 37. The compound of Exemplary Embodiment No. 1, wherein the compound is of Formula XXXV:

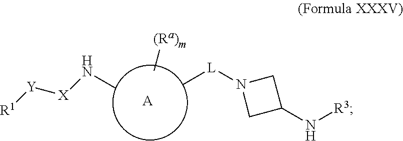

or a pharmaceutically acceptable salt thereof.
Exemplary Embodiment No. 38. The compound of Exemplary Embodiment No. 1, wherein the compound is of Formula XXXVI:

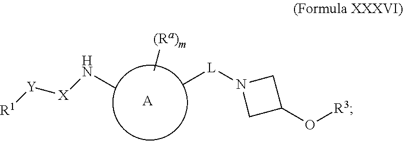

or a pharmaceutically acceptable salt thereof.
Exemplary Embodiment No. 39. The compound of Exemplary Embodiment No. 1, wherein the compound is of Formula XXXVII:

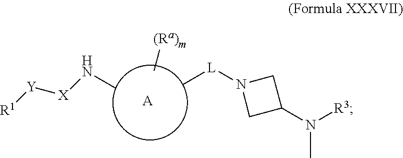

or a pharmaceutically acceptable salt thereof.
Exemplary Embodiment No. 40. The compound of Exemplary Embodiment No. 1, wherein the compound is of Formula XXXVIII:

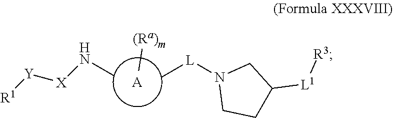

or a pharmaceutically acceptable salt thereof.

Exemplary Embodiment No. 41. The compound of Exemplary Embodiment No. 1, wherein the compound is of Formula XXXIX:

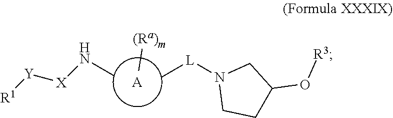

or a pharmaceutically acceptable salt thereof.
Exemplary Embodiment No. 42. The compound of Exemplary Embodiment No. 1, wherein the compound is of Formula XL;

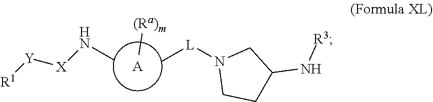

or a pharmaceutically acceptable salt thereof.
Exemplary Embodiment No. 43. The compound of Exemplary Embodiment No. 1, wherein the compound is of Formula XLI:

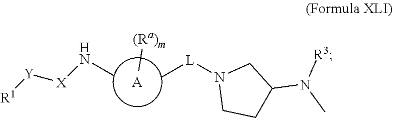

or a pharmaceutically acceptable salt thereof.
Exemplary Embodiment No. 44. The compound of any one of Exemplary Embodiment Nos. 1 to 43, wherein the compound is of Formula XLII:

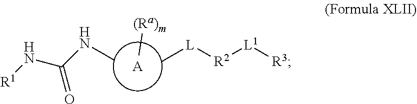

or a pharmaceutically acceptable salt thereof.
Exemplary Embodiment No. 45. The compound of any one of Exemplary Embodiment Nos. 1 to 43, wherein the compound is of Formula XLIII:

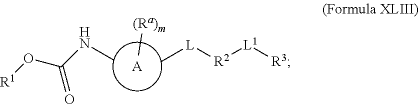

or a pharmaceutically acceptable salt thereof.
Exemplary Embodiment No. 46. The compound of Exemplary Embodiment 1, wherein the compound is of Formula XLIV:

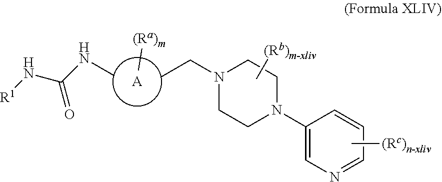

or a pharmaceutically acceptable salt thereof, wherein
m-xliv is 0 to 8; and
n-xliv is 0 to 4.
   Exemplary Embodiment No. 47. The compound of any one of the preceding Exemplary Embodiments, wherein R¹ is C₁₋₆ alkyl optionally substituted by halogen or C₃₋₁₀ cycloalkyl.
   Exemplary Embodiment No. 48. The compound of any one of the preceding Exemplary Embodiments, wherein R¹ is methyl, ethyl, isopropyl, or.

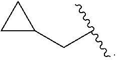 [SMILES: *CC1CC1]

Exemplary Embodiment No. 49. The compound of any one of the preceding Exemplary Embodiments, wherein R¹ is ethyl.
Exemplary Embodiment No. 50. The compound of any one of the preceding Exemplary Embodiments, wherein R¹ is cyclopropyl or cyclobutyl.
Exemplary Embodiment No. 51. The compound of any one of the preceding Exemplary Embodiments, wherein Y is —NH—.
Exemplary Embodiment No. 52. The compound of any one of the preceding Exemplary Embodiments, wherein Y is C₁₋₆ alkylene optionally substituted by one or more halogen, C₁₋₆ alkyl, or two C₁₋₆ alkyl that, together with the atom or atoms to which they are attached, combine to form a C₁₋₆ alkyl.
Exemplary Embodiment No. 53. The compound of any one of the preceding Exemplary Embodiments, wherein X is —C(═O)—.
Exemplary Embodiment No. 54. The compound of any one of the preceding Exemplary Embodiments, wherein Ring A is a 5-membered heteroaryl optionally substituted with one or more R^{a}.
Exemplary Embodiment No. 55. The compound of any one of the preceding Exemplary Embodiments, wherein Ring A is imidazolyl, oxazolyl, thiazolyl, thiophenyl, triazolyl, thiadiazolyl, or furanyl optionally substituted with one or more R^{a}.
Exemplary Embodiment No. 56. The compound of any one of the preceding Exemplary Embodiments, wherein Ring A is a 6-membered heteroaryl optionally substituted with one or more R^{a}.
Exemplary Embodiment No. 57. The compound of any one of the preceding Exemplary Embodiments, wherein Ring A is pyridinyl, pyrimidinyl, or pyridazinyl optionally substituted with one or more R^{a}.
Exemplary Embodiment No. 58. The compound of any one of the preceding Exemplary Embodiments, wherein Ring A is

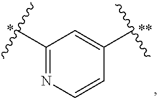 [SMILES: *c1ccnc(*)c1]

wherein [symbol] indicates attachment to —X—NH—, and [symbol] indicates attachment to L.
Exemplary Embodiment No. 59. The compound of any one of the preceding Exemplary Embodiments, wherein Ring A is pyridinyl optionally substituted with one or more R^{a}.
Exemplary Embodiment No. 60. The compound of any one of the preceding Exemplary Embodiments, wherein Ring A is phenyl optionally substituted with one or more R^{a}.
Exemplary Embodiment No. 61. The compound of any one of the preceding Exemplary Embodiments, wherein Ring A is unsubstituted.
Exemplary Embodiment No. 62. The compound of any one of the preceding exemplary embodiments, wherein each R^{a} independently is C₁₋₆ alkyl, C₁₋₆ haloalkyl, —O—C₁₋₆ alkyl, halogen, or two R^{a} combine to form oxo.
Exemplary Embodiment No. 63. The compound of any one of the preceding Exemplary Embodiments, wherein L is bond, —CH₂—, —CH₂—CH₂—, —CH₂—CH₂—CH₂—, —C(O)—, —O—, —S—, —NH—, —N(CH₃)—,

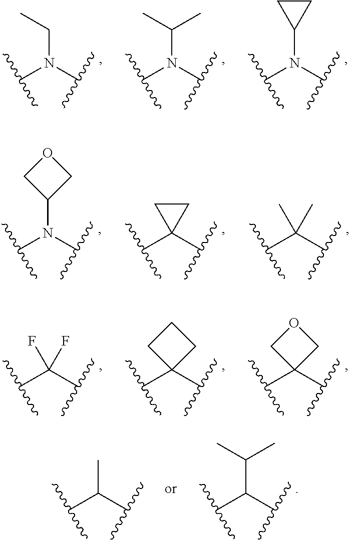

Exemplary Embodiment No. 64. The compound of any one of the preceding Exemplary Embodiments, wherein L is C₁ or C₂ alkylene optionally substituted with oxo.
Exemplary Embodiment No. 65. The compound of any one of the preceding Exemplary Embodiments, wherein L is

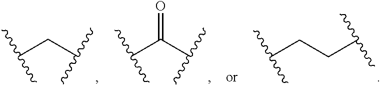

Exemplary Embodiment No. 66. The compound of any one of the preceding Exemplary Embodiments, wherein L is methylene.
Exemplary Embodiment No. 67. The compound of any one of the preceding Exemplary Embodiments, wherein L is

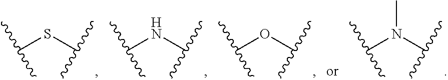

Exemplary Embodiment No. 68. The compound of any one of the preceding Exemplary Embodiments, wherein R² is 3- to 10-membered heterocycloalkylene optionally substituted with one or more R^{b}.
Exemplary Embodiment No. 69. The compound of any one of the preceding Exemplary Embodiments, wherein R² is

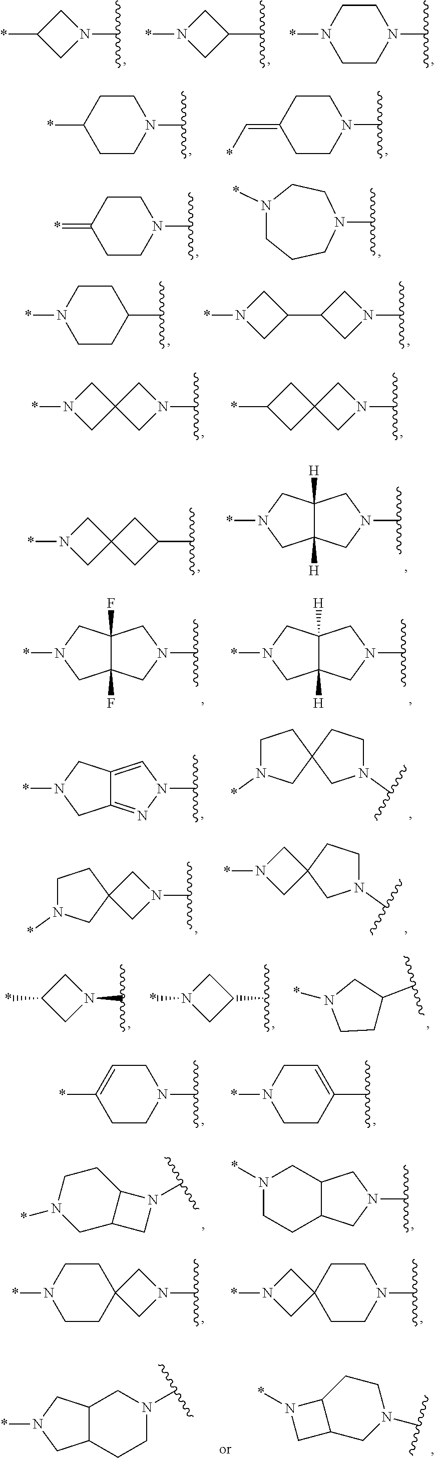

each of which is optionally substituted with one or more R^{b} and wherein * and [symbol] denotes the point of attachment to L and L¹ respectively.
Exemplary Embodiment No. 70. The compound of any one of the preceding Exemplary Embodiments, wherein R² is

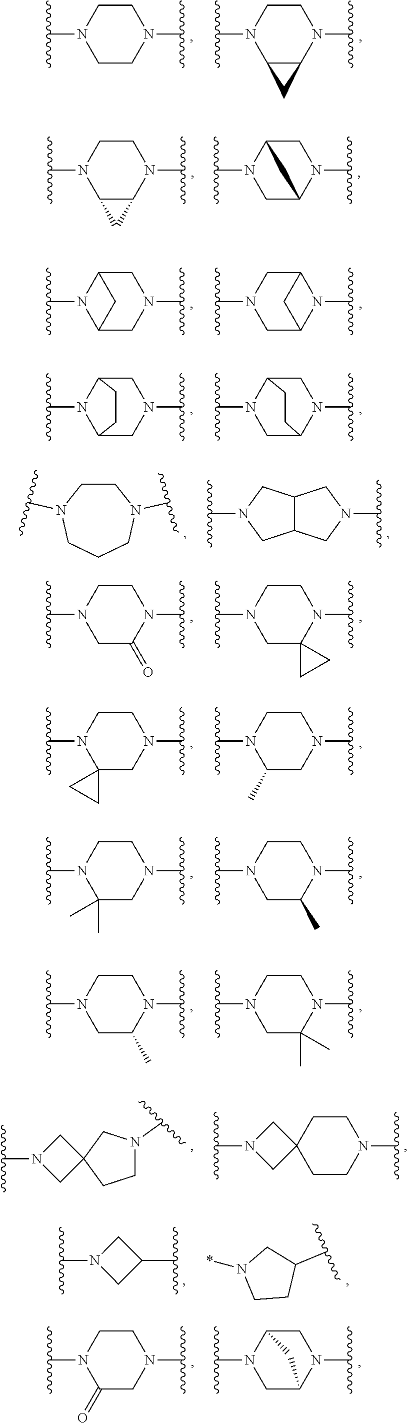

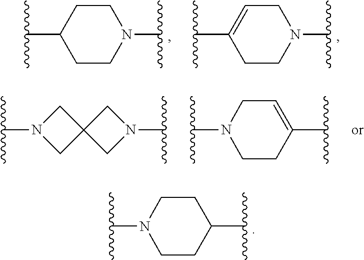

Exemplary Embodiment No. 71. The compound of any one of the preceding Exemplary Embodiments, wherein R² is piperazinyl optionally substituted with one or more R^{b}.
Exemplary Embodiment No. 72. The compound of any one of the preceding Exemplary Embodiments, wherein each R^{b} independently is C₁₋₆ alkyl, or two R^{b} combine to form oxo, or two R^{b}, together with the atom or atoms to which they are attached, combine to form a C₃₋₁₀ cycloalkyl.
Exemplary Embodiment No. 73. The compound of any one of the preceding Exemplary Embodiments, wherein R² is

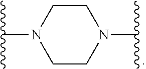 [SMILES: *N1CCN(*)CC1]

Exemplary Embodiment No. 74. The compound of any one of the preceding Exemplary Embodiments, wherein R² is a C₃₋₁₂ cycloalkyl.
Exemplary Embodiment No. 75. The compound of any one of the preceding Exemplary Embodiments, wherein R² is

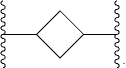 [SMILES: *C1CC(*)C1]

Exemplary Embodiment No. 76. The compound of any one of the preceding Exemplary Embodiments, wherein L¹ is

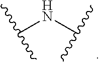 [SMILES: *N*]

Exemplary Embodiment No. 77. The compound of any one of the preceding Exemplary Embodiments, wherein L¹ is —O—.
Exemplary Embodiment No. 78. The compound of any one of the preceding Exemplary Embodiments, wherein L¹ is

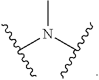 [SMILES: *N(*)C]

Exemplary Embodiment No. 79. The compound of any one of the preceding Exemplary Embodiments, wherein L¹ is a bond.
Exemplary Embodiment No. 80. The compound of any one of the preceding Exemplary Embodiments, wherein R³ is

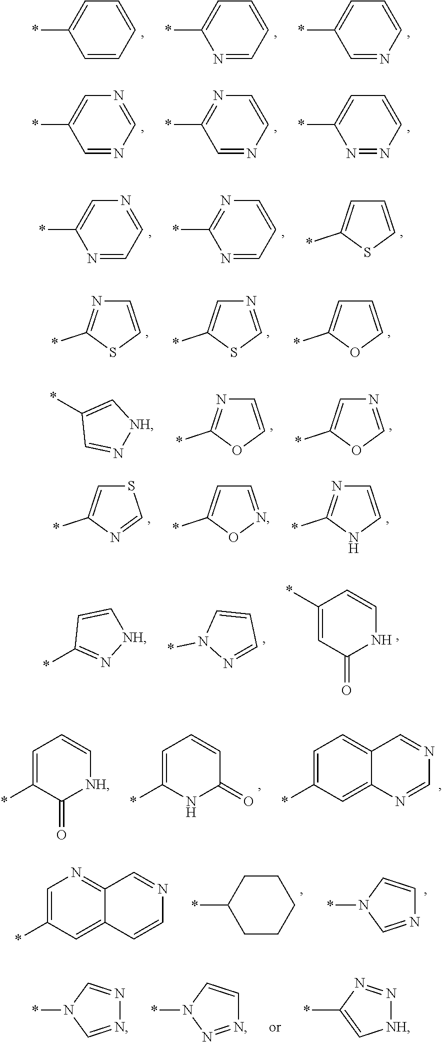

each of which is optionally substituted with one or more R^{c} wherein * denotes point of attachment to L¹.
Exemplary Embodiment No. 81. The compound of any one of the previous Exemplary Embodiments, wherein R³ is

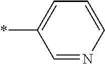 [SMILES: *c1cccnc1]

optionally substituted with one or more R^{c}, wherein * denotes point of attachment to L¹.
Exemplary Embodiment No. 82. The compound of any one of the preceding Exemplary Embodiments, wherein R³ is

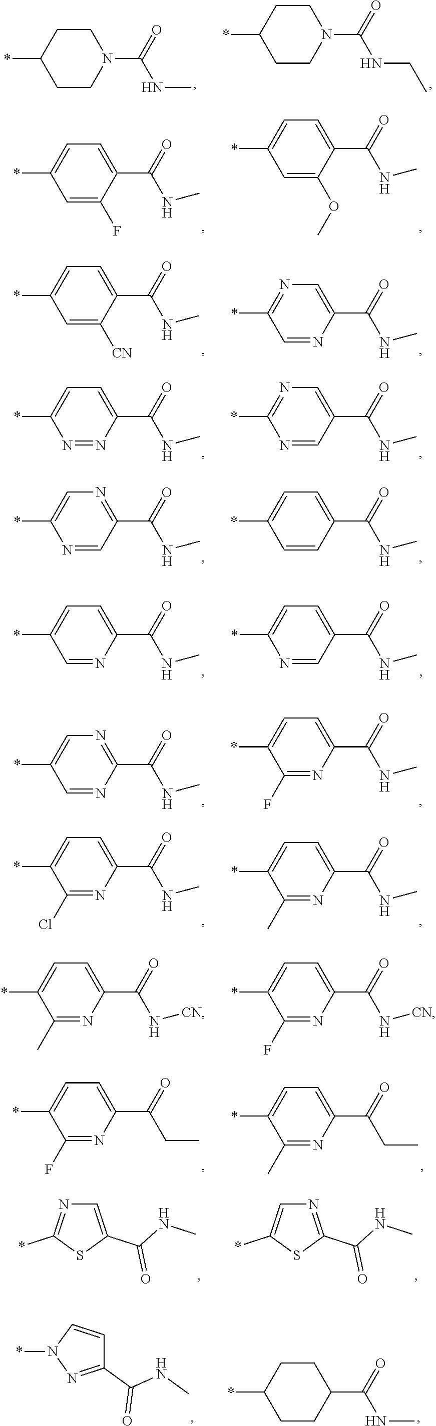

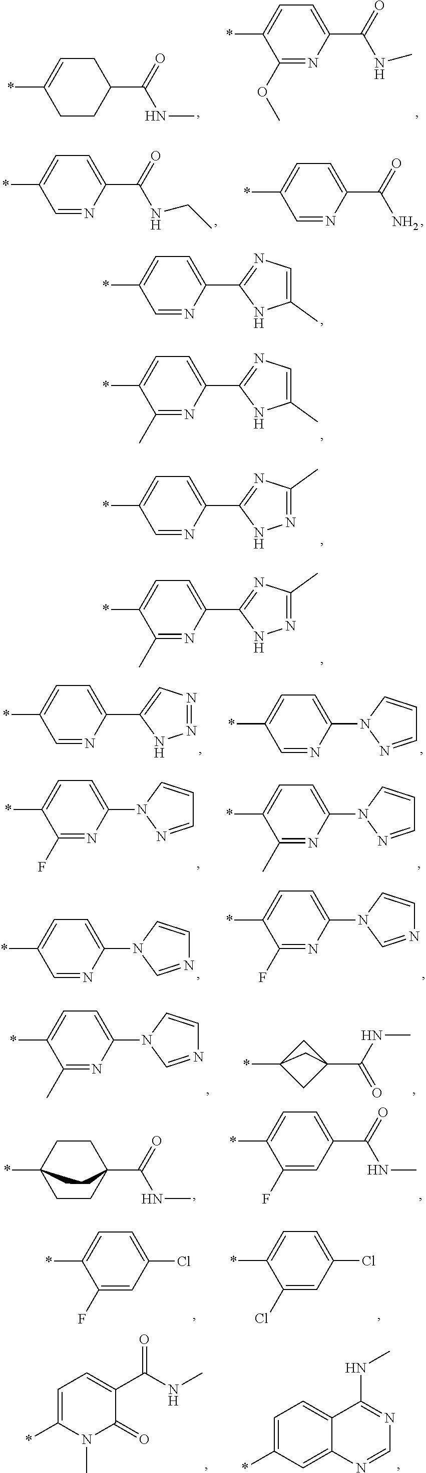

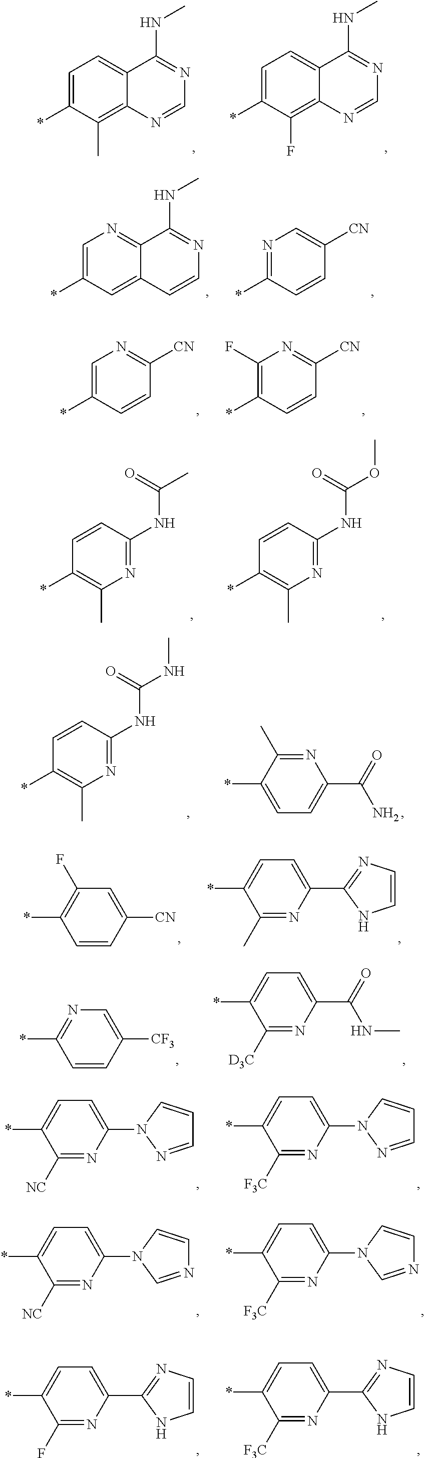

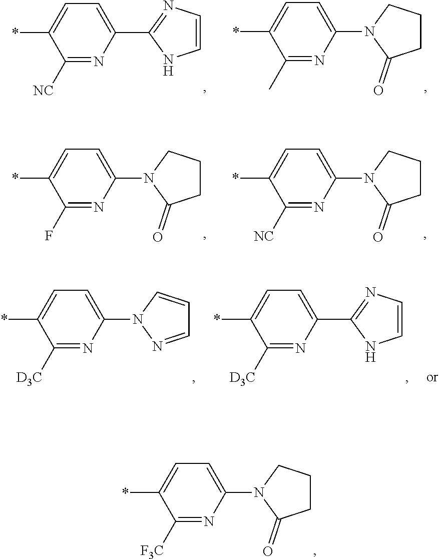

wherein * denotes the point of attachment to L¹.
Exemplary Embodiment No. 83. The compound of any one of the preceding Exemplary Embodiments, wherein R³ is

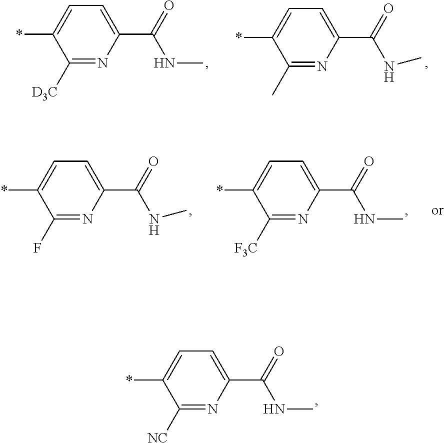

wherein * denotes the point of attachment to L¹.
Exemplary Embodiment No. 84. The compound of any one of the preceding Exemplary Embodiments, wherein R³ is 5- to 10-membered heteroaryl comprising at least one N atom, wherein the heteroaryl is optionally substituted with one or more halogen, CN, —C(═O)—NH₂, —C(═O)—NH—CN, —C(═O)—NH—(C₁₋₆ alkyl), or C₁₋₆ alkyl, wherein the C₁₋₆ alkyl is optionally substituted by one or more halogen or deuterium.
Exemplary Embodiment No. 85. The compound of any one of the preceding Exemplary Embodiments, wherein R³ is pyridinyl optionally substituted with one or more halogen, CN, —C(═O)—NH₂, —C(═O)—NH—CN, —C(═O)—NH—(C₁₋₆ alkyl), or C₁₋₆ alkyl, wherein the C₁₋₆ alkyl is optionally substituted by one or more halogen or deuterium.
Exemplary Embodiment No. 86. The compound of any one of the preceding Exemplary Embodiments, wherein R¹ is

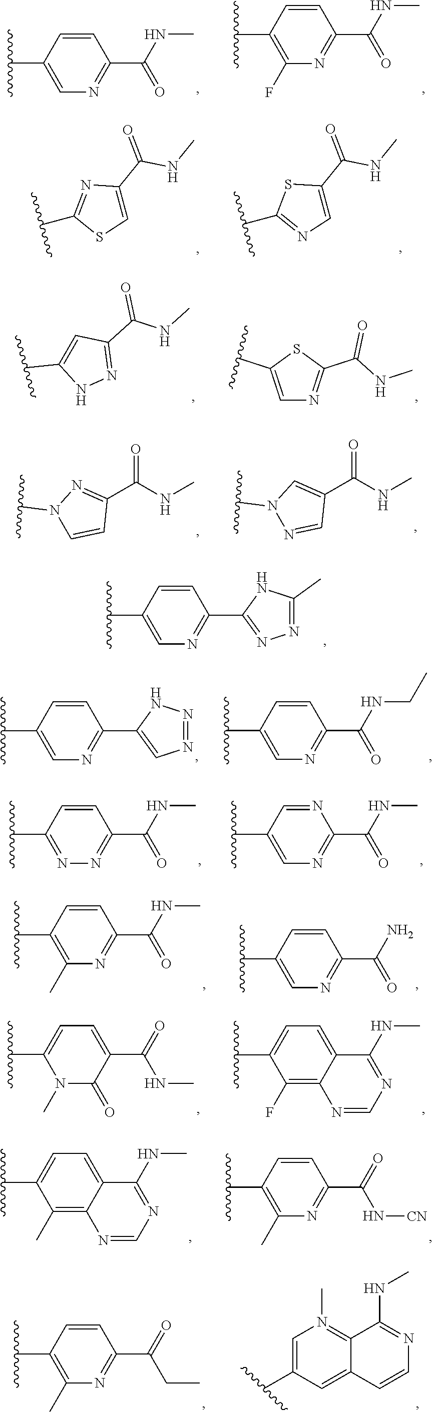

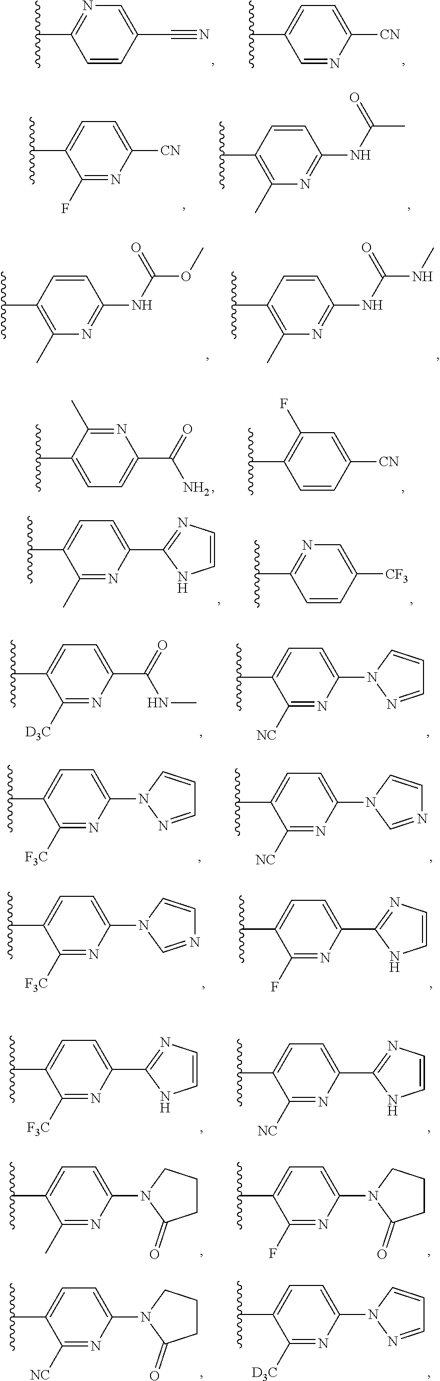

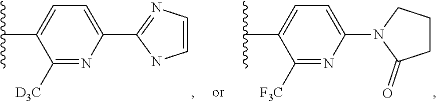 [SMILES: *c1ccc(-c2ncc[nH]2)nc1C([2H])([2H])[2H] ; *c1ccc(N2CCCC2=O)nc1C(F)(F)F]

Exemplary Embodiment No. 87. The compound of any one of the preceding Exemplary Embodiments, wherein R³ is

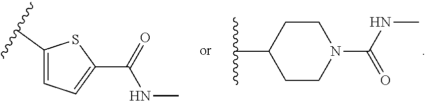

Exemplary Embodiment No. 88. The compound of any one of the preceding Exemplary Embodiments, wherein R³ is C₆₋₁₀ aryl substituted with one or more —C(═O)—NH₂, —C(═O)—NH—CN, or —C(═O)—NH—(C₁₋₆ alkyl).
Exemplary Embodiment No. 89. The compound of any one of the preceding Exemplary Embodiments, wherein R³ is

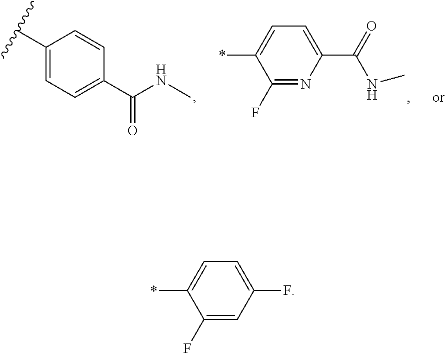 [SMILES: *c1ccc(C(=O)NC)cc1 ; *c1ccc(C(=O)NC)nc1F ; *c1ccc(F)cc1F]

Exemplary Embodiment No. 90. The compound of any one of the previous Exemplary Embodiments, wherein R¹ is halogen, CN, —C(═O)—NH₂, —C(═O)—NH—CN, —C(═O)—NH—(C₁₋₆alkyl), or C₁₋₆ alkyl, wherein the C₁₋₆ alkyl is optionally substituted by one or more halogen or deuterium.
Exemplary Embodiment No. 91. A compound selected from the compounds described in Table 1, or a pharmaceutically acceptable salt thereof.
Exemplary Embodiment No. 92. A pharmaceutical composition comprising the compound of any one of Exemplary Embodiment Nos. 1 to 91.
Exemplary Embodiment No. 93. A method of treating a disease in a subject in need thereof, comprising administering an effective amount of the compound of any one of Exemplary Embodiment Nos. 1 to 91, or the pharmaceutical composition of Exemplary Embodiment No. 92, to the subject in need thereof.
Exemplary Embodiment No. 94. The compound of any one of Exemplary Embodiment Nos. 1 to 91, or the pharmaceutical composition of Exemplary Embodiment No. 92, for use in treating a disease in a subject in need thereof.
Exemplary Embodiment No. 95. Use of the compound of any one of Exemplary Embodiment Nos. 1 to 91, or the pharmaceutical composition of Exemplary Embodiment No. 92, in the manufacture of a medicament for use in treating a disease in a subject in need thereof.
Exemplary Embodiment No. 96. The method, compound, or use of any one of Exemplary Embodiment Nos. 93 to 95, wherein the disease is a cancer.
Exemplary Embodiment No. 97. The method, compound, or use of Exemplary Embodiment No. 96, wherein the cancer is a HR-deficient cancer, a BRCA1- or BRCA2-mutant cancer, or a cancer with a BRCAness phenotype.
Exemplary Embodiment No. 98. The method, compound, or use of Exemplary Embodiment No. 97, wherein the cancer with a BRCAness phenotype is selected from breast cancer, ovarian cancer, prostate cancer, or pancreatic cancer.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 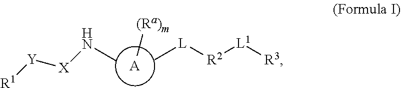
or a pharmaceutically acceptable salt, thereof, wherein
\textcircled{A} is 5- to 10-membered heteroarylene, C₃₋₁₀ cycloalkylene, C₆₋₁₀ arylene, or 3- to 10-membered heterocycloalkylene;
m is 0, 1, 2, 3, 4, or 5;
each R^{a} independently is halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{a} combine to form oxo, wherein the —O—(C₁₋₆ alkyl), —NH(C₁₋₆ alkyl), —N(C₁₋₆ alkyl)₂, or —C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{a}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃-10 cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
X is —C(═O)—, —S(═O)₂—;
Y is a bond, —O—, —S—, —NH—, or C₁₋₆ alkylene, wherein the C₁₋₆ alkylene is optionally substituted with one or more oxo, halogen, —CN, —OH, —NH₂, C₁₋₆ alkyl, or two C₁₋₆ alkyl that combine, with the atom or atoms to which they are attached, to form a C₃₋₁₀ cycloalkyl group;
R¹ is —C₁₋₆ alkyl, H, halogen, —CN, —OH, —NH₂, —O—(C₁₋₆ alkyl), —NH(C₁₋₆ alkyl), —N(C₁₋₆ alkyl)(C₂₋₆ alkyl) C₃₋₁₀ cycloalkyl, C₆₋₁₀ aryl, 3- to 10-membered heterocycloalkyl, or 5- to 10-membered heteroaryl, wherein the —C₁₋₆ alkyl, —O—(C₁₋₆ alkyl), —NH(C₁₋₆ alkyl), or —N(C₁₋₆ alkyl)₂, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, or 5- to 10-membered heteroaryl, and the C₃₋₁₀ cycloalkyl, C₆₋₁₀ aryl, 3- to 10-membered heterocycloalkyl, or 5- to 10-membered heteroaryl is optionally substituted with one or more oxo, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
L is C₁₋₆ alkylene, a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R² is 3- to 10-membered heterocycloalkylene, C₃₋₁₀ cycloalkylene, C₆₋₁₀ arylene, or 5- to 10-membered heteroarylene, wherein the C₃₋₁₀ cycloalkylene, C₆₋₁₀ arylene, 3- to 10-membered heterocycloalkylene, or 5- to 10-membered heteroarylene is optionally substituted with one or more R^{b};
each R^{b} independently is halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{b} combine to form oxo, wherein the —O—(C₁₋₆ alkyl), —NH(C₁₋₆ alkyl), —N(C₁₋₆ alkyl)₂, C₁₋₆ alkyl, is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl, and the C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{b}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃-10 cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
L¹ is a bond, —O—, —S—, —S(═O)—, —S(═O)₂—, —NH—, —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene, wherein the —N(C₁₋₆ alkyl)-, —N(C₃₋₆ cycloalkyl)-, —N(C₃₋₆ heterocycloalkyl)-, C₁₋₆ alkylene, C₂₋₆ alkenylene, C₂₋₆ alkynylene, C₃₋₆ cycloalkylene, or C₃₋₆ heterocycloalkylene is optionally substituted with one or more oxo, deuterium, halogen, —CN, —OH, or —NH₂;
R³ is 5- to 10-membered heteroaryl, C₃₋₁₀ cycloalkyl, C₆₋₁₀ aryl, or 3- to 10-membered heterocycloalkyl, wherein the C₃₋₁₀ cycloalkyl, C₆₋₁₀ aryl, 3- to 10-membered heterocycloalkyl, or 5- to 10-membered heteroaryl is optionally substituted with one or more R^{c};
each R^{c} independently is halogen, —CN, —OH, —NH₂, —C(═O)—H, —C(═O)—NH₂, —NH—C(═O)—H, —NH—(C═O)—NH₂, —C(═O)—CN, —NH—C(═O)—CN, —C(═O)—NH—CN, —O—(C₁₋₆ alkyl), —NH(C₁₋₆ alkyl), —N(C₁₋₆ alkyl)₂, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), —NH—(C═O)—O—(C₁₋₆ alkyl), —C(═O)—NH—(C₃₋₁₀ cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, or two R^{c} combine to form oxo, wherein the —O—(C₁₋₆ alkyl), —NH(C₁₋₆ alkyl), —N(C₁₋₆ alkyl)₂, —C(═O)—(C₁₋₆ alkyl), —C(═O)—NH—(C₁₋₆ alkyl), —NH—C(═O)—(C₁₋₆ alkyl), —NH—(C═O)—NH—(C₁₋₆ alkyl), or —NH—(C═O)—O—(C₁₋₆ alkyl), is optionally substituted with one or more oxo, deuterium, halogen, —CN, —OH, —NH₂, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl —C(═O)—NH—(C₃-10 cycloalkyl), —C(═O)—NH-(3- to 10-membered heterocycloalkyl), C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, —NH₂, C₁₋₆ alkyl, C₁₋₆ haloalkyl, —O—C₁₋₆ alkyl, —NH—C₁₋₆ alkyl, —N(C₁₋₆ alkyl)₂, C₃₋₁₀ cycloalkyl, or 3- to 10-membered heterocycloalkyl, C₆₋₁₀ aryl, or 5- to 10-membered heteroaryl;
or two R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂;
or one R^{b} and one R^{c}, together with the intervening atoms they are attached to, form C₃₋₁₀ cycloalkyl, C₆₋₁₀ aryl, 3- to 10-membered heterocycloalkyl, or 5- to 10-membered heteroaryl, wherein the C₃₋₁₀ cycloalkyl, C₆₋₁₀ aryl, 3- to 10-membered heterocycloalkyl, or 5- to 10-membered heteroaryl is optionally substituted with one or more R^{bc}; and
each R^{bc} independently is oxo, deuterium, halogen, —CN, —OH, —NH₂, —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl, wherein the —O—(C₁₋₆ alkyl), —NH(C₁₋₆ alkyl), —N(C₁₋₆ alkyl)₂, C₁₋₆ alkyl, C₂₋₆ alkenyl, C₂₋₆ alkynyl, C₃₋₁₀ cycloalkyl, C₆₋₁₀ aryl, 3- to 10-membered heterocycloalkyl, or 5- to 10-membered heteroaryl is optionally substituted with one or more oxo, deuterium, halogen, —CN, —OH, or —NH₂.

2. The compound of claim 1, wherein the compound is of Formula II: 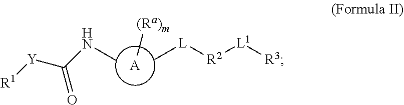
or a pharmaceutically acceptable salt thereof.

3. The compound of claim 1, wherein the compound is of Formula V′: 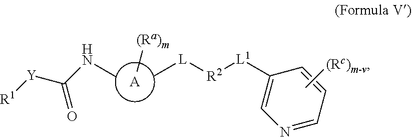
or a pharmaceutically acceptable salt thereof;
wherein m-v is 0 to 4.

4. The compound of claim 1, wherein the compound is of Formula XXXI′: 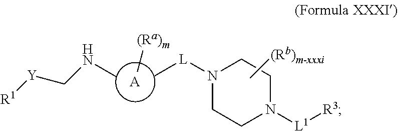
or a pharmaceutically acceptable salt thereof;
wherein m-xxxi is 0 to 8.

5. The compound of claim 1, wherein the compound is of Formula VII′: 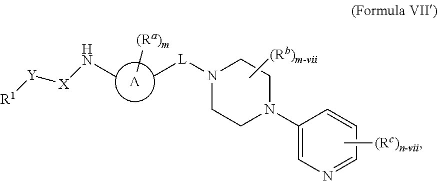
or a pharmaceutically acceptable salt thereof;
wherein m-vii is 0 to 8; and
n-vii is 0 to 4.

6. The compound of claim 1, wherein the compound is of Formula XLIV: 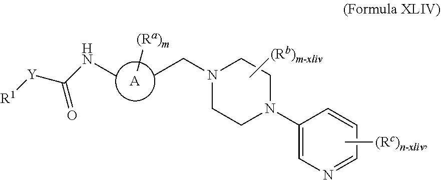
or a pharmaceutically acceptable salt thereof; wherein
m-xliv is 0 to 8; and
n-xliv is 0 to 4.

7. The compound of claim 1, wherein R¹ is C₁₋₆ alkyl optionally substituted by halogen or C₃₋₁₀ cycloalkyl.

8. The compound of claim 1, wherein R¹ is methyl, ethyl, isopropyl, or

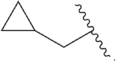 [SMILES: *CC1CC1]

9. The compound of claim 1, wherein R¹ is cyclopropyl or cyclobutyl.

10. The compound of claim 1, wherein Y is —NH—.

11. The compound of claim 1, wherein Y is C₁₋₆ alkylene optionally substituted by one or more halogen, C₁₋₆ alkyl, or two C₁₋₆ alkyl that, together with the atom or atoms to which they are attached, combine to form a C₁₋₆ alkyl.

12. The compound of claim 1, wherein X is —C(═O)—.

13. The compound of claim 1, wherein Ring A is a 5-membered or 6-membered heteroaryl optionally substituted with one or more R^{a}.

14. The compound of claim 1, wherein Ring A is pyridinyl, pyrimidinyl, or pyridazinyl optionally substituted with one or more R^{a}.

15. The compound of claim 1, wherein Ring A is 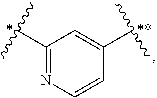 [SMILES: *c1ccnc(*)c1]
wherein [symbol] indicates attachment to —X—NH—, and [symbol] indicates attachment to L.

16. The compound of claim 1, wherein Ring A is imidazolyl, oxazolyl, thiazolyl, thiophenyl, triazolyl, thiadiazolyl, or furanyl optionally substituted with one or more R^{a}.

17. The compound of claim 1, wherein each R^{a} independently is C₁₋₆ alkyl, C₁₋₆ haloalkyl, —O—C₁₋₆ alkyl, halogen, or two R^{a} combine to form oxo.

18. The compound of claim 1, wherein L is C₁ or C₂ alkylene optionally substituted with oxo.

19. The compound of claim 1, wherein L is

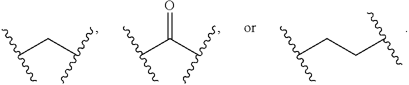

20. The compound of claim 1, wherein R² is 3- to 10-membered heterocycloalkylene optionally substituted with one or more R^{b}.

21. The compound of claim 1, wherein R² is 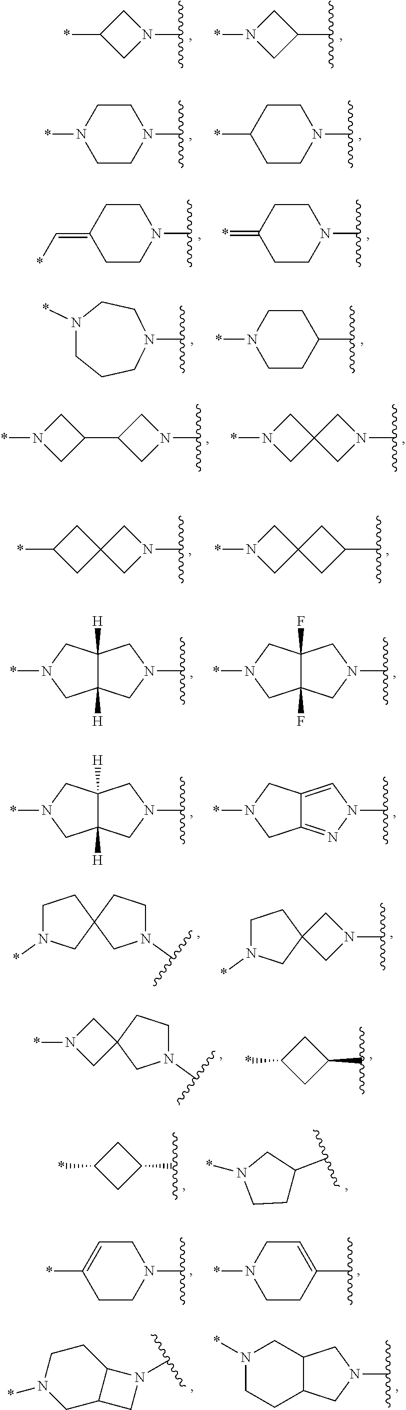 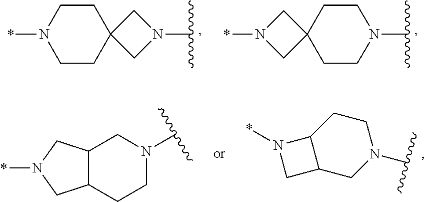
each of which is optionally substituted with one or more R^{b} and wherein * and [symbol] denotes the point of attachment to L and L¹ respectively.

22. The compound of claim 1, wherein each R^{b} independently is C₁₋₆ alkyl, or two R^{b} combine to form oxo, or two R^{b}, together with the atom or atoms to which they are attached, combine to form a C₃₋₁₀ cycloalkyl.

23. The compound of claim 1, wherein R² is piperazinyl optionally substituted with one or more R^{b}.

24. The compound of claim 1, wherein R² is

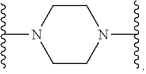 [SMILES: *N1CCN(*)CC1]

25. The compound of claim 1, wherein L¹ is a bond.

26. The compound of claim 1, wherein R³ is 5- to 10-membered heteroaryl comprising at least one N atom, wherein the heteroaryl is optionally substituted with one or more halogen, CN, —C(═O)—NH₂, —C(═O)—NH—CN, —C(═O)—NH—(C₁₋₆ alkyl), or C₁₋₆ alkyl, wherein the C₁₋₆ alkyl is optionally substituted by one or more halogen or deuterium.

27. The compound of claim 1, wherein R³ is pyridinyl optionally substituted with one or more halogen, CN, —C(═O)—NH₂, —C(═O)—NH—CN, —C(═O)—NH—(C₁₋₆ alkyl), or C₁₋₆ alkyl, wherein the C₁₋₆ alkyl is optionally substituted by one or more halogen or deuterium.

28. The compound of claim 1, wherein R³ is R³ 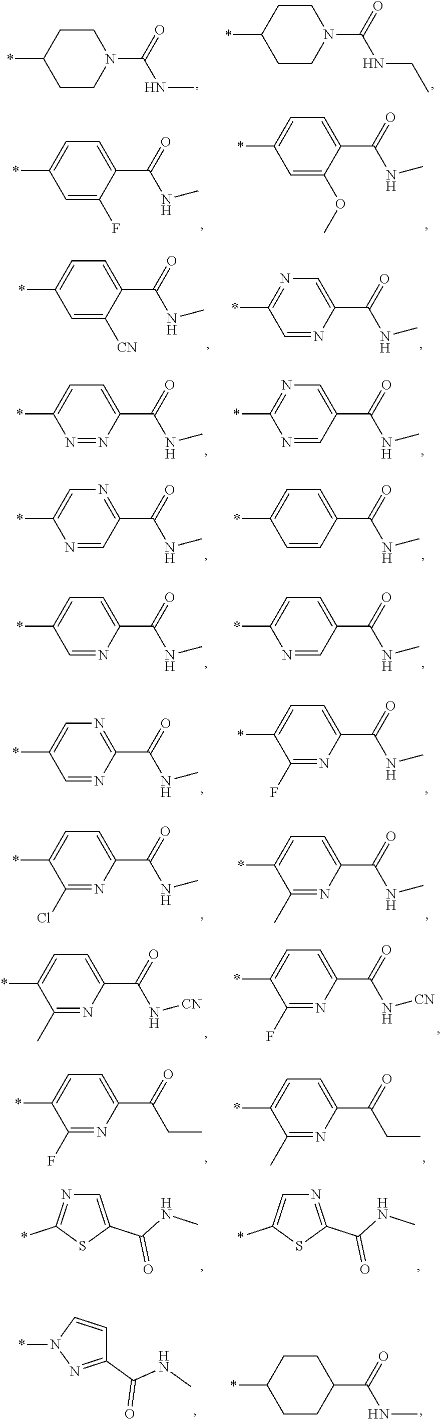 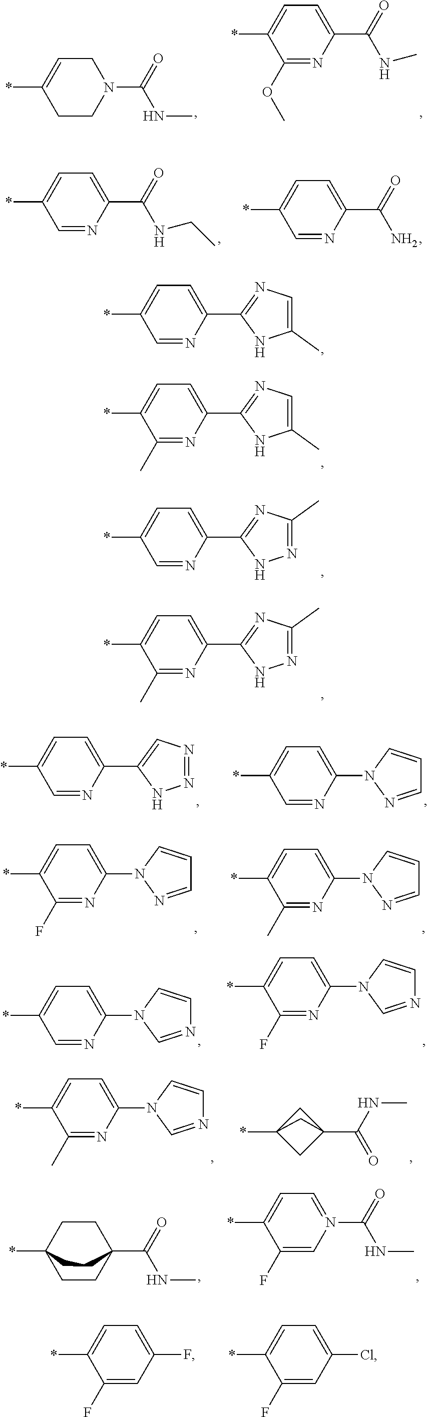 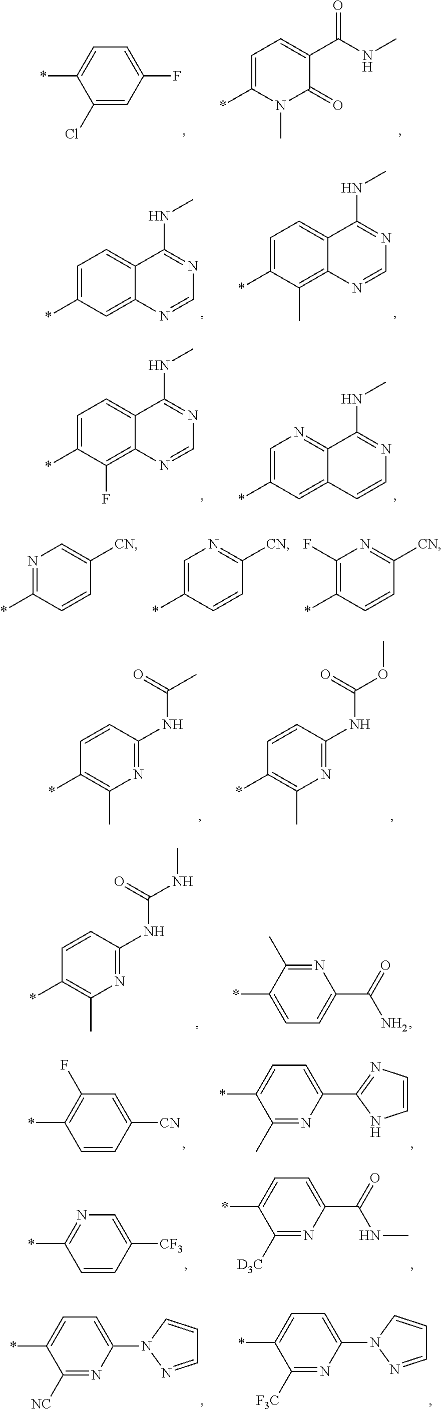 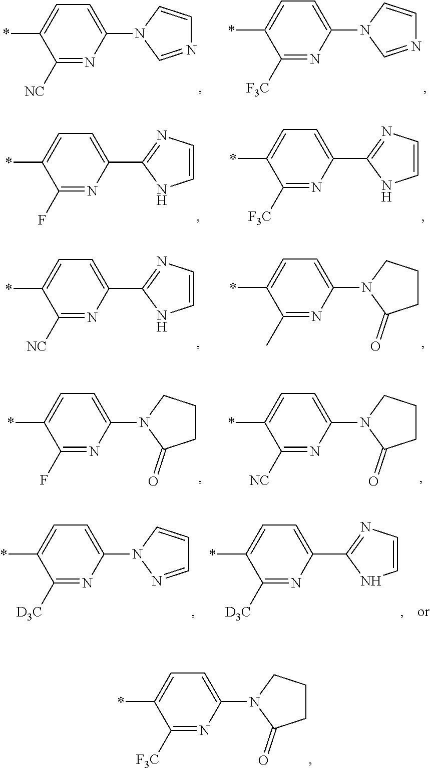
wherein * denotes the point of attachment to L¹.

29. The compound of claim 1, wherein R³ is 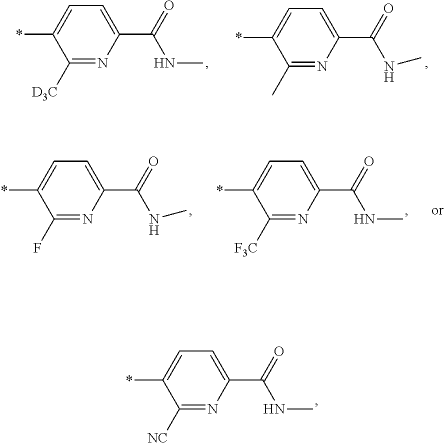
wherein * denotes the point of attachment to L¹.

30. The compound of claim 1, wherein R^{c} is halogen, —C(═O)—NH₂, —C(═O)—NH—CN, —C(═O)—NH—(C₁₋₆ alkyl), or C₁₋₆ alkyl, wherein the C₁₋₆ alkyl is optionally substituted by one or more halogen or deuterium.

31. A compound selected from the compounds described in Table 1, or a pharmaceutically acceptable salt thereof.

32. A pharmaceutical composition comprising the compound of claim 1.

33. A method of treating a disease in a subject in need thereof, comprising administering an effective amount of the compound of claim 1, to the subject.

34-35. (canceled)

36. The method of claim 33, wherein the disease is cancer.

37. The method of claim 36, wherein the cancer is a HR-deficient cancer, a BRCA1- or BRCA2-mutant cancer, or a cancer with a BRCAness phenotype.

38. The method of claim 37, wherein the cancer with a BRCAness phenotype is selected from breast cancer, ovarian cancer, prostate cancer, or pancreatic cancer.
